(19) United States
(12) Patent Application Publication
(10) Pub. No.: US 2025/0263392 A1
(43) Pub. Date: Aug. 21, 2025

(54) AMINOBENZAZEPINE COMPOUNDS, IMMUNOCONJUGATES, AND USES THEREOF

(71) Applicant: BOLT BIOTHERAPEUTICS, INC., Redwood City, CA (US)

(72) Inventors: Shelley Erin Ackerman, Redwood City, CA (US); Michael N. Alonso, Redwood City, CA (US); Romas Kudirka, Redwood City, CA (US); Arthur Lee, Redwood City, CA (US); Brian Safina, Redwood City, CA (US); Matthew Zhou, Redwood City, CA (US)

(73) Assignee: BOLT BIOTHERAPEUTICS, INC., Redwood City, CA (US)

(21) Appl. No.: 19/203,992

(22) Filed: May 9, 2025

Related U.S. Application Data
(62) Division of application No. 16/900,193, filed on Jun. 12, 2020.
(60) Provisional application No. 62/963,884, filed on Jan. 21, 2020.
(60) Provisional application No. 62/861,139, filed on Jun. 13, 2019.

(51) Int. Cl. C07D 401/12; A61K 47/68; A61P 35/00; C07K 16/28; C07K 16/32

(52) U.S. Cl. CPC C07D 401/12; A61K 47/6803; A61K 47/6849; A61P 35/00; C07K 16/2827; C07K 16/32

(57) ABSTRACT

The invention provides immunoconjugates of Formula I or III comprising an antibody linked by conjugation to one or more aminobenzazepine derivatives. The invention also provides aminobenzazepine derivative intermediate compositions of Formula II comprising a reactive functional group. Such intermediate compositions are suitable substrates for formation of the immunoconjugates through a linker or linking moiety. The invention further provides methods of treating cancer with the immunoconjugates.

### CROSS REFERENCE TO RELATED APPLICATIONS

This divisional application claims the benefit of priority to non-provisional application U.S. Ser. No. 16/900,193, filed 12 Jun. 2020, which claims the benefit of priority to U.S. Provisional Application No. 62/963,884, filed 21 Jan. 2020, and U.S. Provisional Application No. 62/861,139, filed 13 Jun. 2019, each of which are incorporated by reference in their entirety.

### SEQUENCE LISTING

The instant application contains a Sequence Listing which has been submitted electronically in XML. format and is hereby incorporated by reference in its entirety. Said XML. copy, created on 27 Feb. 2023, is named txt_17019.002US2 and is 794,176 bytes in size.

### FIELD OF THE INVENTION

The invention relates generally to an immunoconjugate comprising an antibody conjugated to one or more aminobenzazepine molecules.

### BACKGROUND OF THE INVENTION

New compositions and methods for the delivery of antibodies and immune adjuvants are needed in order to reach inaccessible tumors and/or to expand treatment options for cancer patients and other subjects. The invention provides such compositions and methods.

### SUMMARY OF THE INVENTION

The invention is generally directed to immunoconjugates comprising an antibody linked by conjugation to one or more aminobenzazepine derivatives. The invention is further directed to aminobenzazepine derivative intermediate compositions comprising a reactive functional group. Such intermediate compositions are suitable substrates for formation of immunoconjugates wherein an antibody may be covalently bound to one or more aminobenzazepine derivatives, through a linker or linking moiety. The invention is further directed to use of such an immunoconjugates in the treatment of an illness, in particular cancer.

An aspect of the invention is an immunoconjugate comprising an antibody covalently attached to a linker which is covalently attached to one or more aminobenzazepine moieties.

Another aspect of the invention is an aminobenzazepine-linker compound.

Another aspect of the invention is a method for treating cancer comprising administering a therapeutically effective amount of an immunoconjugate comprising an antibody linked by conjugation to one or more aminobenzazepine moieties.

Another aspect of the invention is a use of an immunoconjugate comprising an antibody linked by conjugation to one or more aminobenzazepine moieties for treating cancer.

Another aspect of the invention is a method of preparing an immunoconjugate by conjugation of one or more aminobenzazepine moieties with an antibody.

### BRIEF DESCRIPTION OF THE DRAWINGS

FIGS. 1A-D show heavy chain and light chain CDRs of PD-L1 Type A binding agents 1-42.

FIGS. 2A-D show first (HFW1), second (HFW2), third (HFW3), and fourth (HFW4) heavy chain framework region polypeptides of PD-L1 Type A binding agents 1-42.

FIGS. 3A-D show first (LFW1), second (LFW2), third (LFW3), and fourth (LFW4) light chain framework region polypeptides of PD-L1 Type A binding agents 1-42.

FIGS. 4 A-D show heavy chain variable region (VH) of PD-L1 Type A binding agents 1-42.

FIGS. 4 E-G show light chain variable region (VL) of PD-L1 Type A binding agents 1-42.

FIGS. 5A-B show heavy chain and light chain CDRs of PD-L1 Type B binding agents 1-21.

FIGS. 6A-B show first (HFW1), second (HFW2), third (HFW3), and fourth (HFW4) heavy chain framework region polypeptides of PD-L1 Type B binding agents 1-21.

FIGS. 7A-B show first (LFW1), second (LFW2), third (LFW3), and fourth (LFW4) light chain framework region polypeptides of PD-L1 Type B binding agents 1-21.

FIGS. 8A-B show heavy chain variable region (VH) of PD-L1 Type B binding agents 1-21.

FIGS. 8C-D show light chain variable region (VL) of PD-L1 Type B binding agents 1-21.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The term “immunoconjugate” refers to an antibody construct that is covalently bonded to an adjuvant moiety via a linker. the term “adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

“Adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

The terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

“Antibody” refers to a polypeptide comprising an antigen binding region (including the complementarity-determining regions (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

“Antibody construct” refers to an antibody or a fusion protein comprising (i) an antigen binding domain and (ii) an Fc domain.

In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a t-cell receptor, so as to provide a T-cell receptor like construct that binds PD-L1. In yet another embodiment, the antibody fragment is part of a bispecific T-cell engager (BiTEs) comprising a CD1 or CD3 binding domain and linker.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The binding agent comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The binding agent can be any of a variety of types of binding agents known in the art that comprise Ig heavy and light chains. For instance, the binding agent can be an antibody, an antigen-binding antibody “fragment,” or a T-cell receptor.

“Biosimilar” refers to an approved antibody construct that has active properties similar to, for example, a PD-L1-targeting antibody construct previously approved such as atezolizumab (TECENTRIQ™, Genentech, Inc.), durvalumab (IMFINZI™, AstraZeneca), and avelumab (BAVENCIO™, EMD Serono, Pfizer); a HER2-targeting antibody construct previously approved such as trastuzumab (HERCEPTIN™, Genentech, Inc.), and pertuzumab (PERJETA™, Genentech, Inc.); or a CEA-targeting antibody such as labetuzumab (CEA-CIDE™, MN-14, hMN14, Immunomedics) CAS Reg. No. 219649-07-7).

“Biobetter” refers to an approved antibody construct that is an improvement of a previously approved antibody construct, such as atezolizumab, durvalumab, avelumab, trastuzumab, pertuzumab, and labetuzumab. The biobetter can have one or more modifications (e.g., an altered glycan profile, or a unique epitope) over the previously approved antibody construct.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, D and L stereoisomers where they exist of alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a functional group that covalently bonds two or more moieties in a compound or material. For example, the linking moiety can serve to covalently bond an adjuvant moiety to an antibody construct in an immunoconjugate.

“Linking moiety” refers to a functional group that covalently bonds two or more moieties in a compound or material. For example, the linking moiety can serve to covalently bond an adjuvant moiety to an antibody in an immunoconjugate. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

A wavy line ( [symbol] ) or an asterisk (*) represents a point of attachment of the specified chemical moiety. If the specified chemical moiety has two wavy lines ( [symbol] ) present, it will be understood that a divalent chemical moiety can be used bilaterally, i.e., as read from left to right or from right to left. In some embodiments, a specified moiety having two wavy lines ( [symbol] ) present is considered to be used as read from left to right.

“Alkyl” refers to a straight or branched, saturated, aliphatic radical having the number of carbon atoms indicated. Alkyl can include any number of carbons. For example, C₁-C₄ alkyl includes, but is not limited to, methyl, ethyl, propyl, isopropyl, butyl, isobutyl, sec-butyl, and tert-butyl. Alkyl can also refer to alkyl groups having up to 30 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical.

“Cycloalkyl” refers to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to an aromatic ring system having any suitable number of ring atoms and any suitable number of rings. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

“Heterocycloalkyl” and “heteroaryl” refer to a “cycloalkyl” or “aryl” group as described herein, wherein one or more carbon atoms are optionally and independently replaced with heteroatom selected from N, O, and S. “Heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, oxo (═O), alkylamino, amido, acyl, nitro, cyano, and alkoxy.

The term “heterocycloalkyldiyl” refers to a divalent heterocycloalkyl radical.

Heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

The term “heteroaryldiyl” refers to a divalent heteroaryl radical.

“Heterocycloalkyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted.

Heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

The term “heterocycloalkyldiyl” refers to a divalent heterocycloalkyl radical.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

“PD-L1 expression” refers to a cell that has a PD-L1 receptor on the cell's surface. As used herein “PD-L1 overexpression” refers to a cell that has more PD-L1 receptors as compared to corresponding non-cancer cell.

“HER2” refers to the protein human epidermal growth factor receptor 2.

“HER2 expression” refers to a cell that has a HER2 receptor on the cell's surface. For example, a cell may have from about 20,000 to about 50,000 HER2 receptors on the cell's surface. As used herein “HER2 overexpression” refers to a cell that has more than about 50,000 HER2 receptors. For example, a cell 2, 5, 10, 100, 1,000, 10,000, 100,000, or 1,000,000 times the number of HER2 receptors as compared to corresponding non-cancer cell (e.g., about 1 or 2 million HER2 receptors). It is estimated that HER2 is overexpressed in about 25% to about 30% of breast cancers.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as an immunoconjugate that produces therapeutic effects for which it is administered. The exact dose will depend on the purpose of the treatment, and will be ascertainable by one skilled in the art using known techniques (see, e.g., Lieberman, PharmaceuticalDosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the immunoconjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the immunoconjugate may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety. Further, a decreased amount of the immunoconjugate may be administered (as measured by the total number of antibody constructs or the total number of adjuvants administered as part of the immunoconjugate) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

### Antibodies

The immunoconjugate of the invention comprises an antibody. Included in the scope of the embodiments of the invention are functional variants of the antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells expressing PD-L1, HER2 or CEA to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

The antibodies comprising the immunoconjugates of the invention include Fc engineered variants. In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E345R, E233, G237, P238, H268, P271, L328 and A330. Additional Fc region modifications for modulating Fc receptor binding are described in, for example, U.S. Patent Application Publication 2016/0145350 and U.S. Pat. Nos. 7,416,726 and 5,624,821, which are hereby incorporated by reference in their entireties herein.

The antibodies comprising the immunoconjugates of the invention include glycan variants, such as afucosylation. In some embodiments, the Fc region of the binding agents are modified to have an altered glycosylation pattern of the Fc region compared to the native non-modified Fc region.

Amino acid substitutions of the inventive antibody constructs or antigen binding domains are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

Methods for generating antibodies are described in, for example, Kohler and Milstein, Eur. J. Immunol., 5: 511-519 (1976); Harlow and Lane (eds.), Antibodies: A Laboratory Manual, CSH Press (1988); and Janeway et al. (eds.), Immunobiology, 9th Ed., Garland Publishing, New York, NY (2017). In certain embodiments, a human or chimeric antibody or antibody fragment can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, New Jersey (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008) and use of phage display (see, e.g., Fellouse, et al., Journal of Molecular Biology, 373(4): 924-940 (2007) and Glanville, et al., PNAS, 106(48): 20216-20221 (2009)).

In an exemplary embodiment, the immunoconjugates of the invention comprise an antibody construct that comprises an antigen binding domain that specifically recognizes and binds PD-L1.

Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80) and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers, or infections. PD-L1/PD-1 pathway also contributes to preventing autoimmunity and therefore agonistic agents against PD-L1 or agents that deliver immune inhibitory payloads may help treatment of autoimmune disorders.

Several antibodies targeting PD-L1 have been developed for the treatment of cancer, including atezolizumab (TECENTRIQ™), durvalumab (IMFINZI™), and avelumab (BAVENCIO™). Nevertheless, there continues to be a need for new PD-L1-binding agents, including agents that bind PD-L1 with high affinity and effectively prevent PD-L1/PD-1 signaling and agents that can deliver therapeutic payloads to PD-L1 expressing cells. In addition, there is a need for new PD-L1-binding agents to treat autoimmune disorders and infections.

A method is provided of delivering an aminobenzazepine derivative payload to a cell expressing PD-L1 comprising administering to the cell, or mammal comprising the cell, an immunoconjugate comprising an anti-PD-L1 antibody covalently attached to a linker which is covalently attached to one or more aminobenzazepine moieties.

Also provided is a method for enhancing or reducing or inhibiting an immune response in a mammal, and a method for treating a disease, disorder, or condition in a mammal that is responsive to PD-L1 inhibition, which methods comprise administering a PD-L1 immunoconjugate thereof, to the mammal.

The invention provides a PD-L1 binding agent comprising an immunoglobulin heavy chain variable region polypeptide and an immunoglobulin light chain variable region polypeptide.

The PD-L1 binding agent specifically binds PD-L1. The binding specificity of the agent allows for targeting PD-L1 expressing cells, for instance, to deliver therapeutic payloads to such cells.

In some embodiments, the PD-L1 binding agent (Type A or Type B) binds to human PD-L1, for example, a protein comprising SEQ ID NO: 307. However, binding agents that bind to any PD-L1 homolog or paralog also are encompassed. In some embodiments, the PD-L1 protein comprises at least about 70%, about 75%, about 80%, about 85%, about 90%, about 91%, about 92%, about 93%, about 94%, about 95%, about 96%, about 97%, about 98%, about 99%, or more sequence identity to SEQ ID NO: 307. In some embodiments, the binding agent binds human PD-L1 and cynomolgus PD-L1; or human, cynomolgus and mouse PD-L1.

SEQ ID NO: 307
MRIFAVFIFMTYWHLLNAFTVTVPKDLYVVEYGSNMTIECKFPVEKQLDL
AALIVYWEMEDKNITQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET

In some embodiments, the PD-L1 binding agent binds PD-L1 without substantially inhibiting or preventing PD-L1 from binding to its receptor, PD-1. However, in other embodiments, the PD-L1 binding agent can completely or partially block (inhibit or prevent) binding of PD-L1 to its receptor, PD-1, such that the antibody can be used to inhibit PD-L1/PD-1 signaling (e.g., for therapeutic purposes).

The antibody or antigen-binding antibody fragment can be monospecific for PD-L1, or can be bispecific or multi-specific. For instance, in bivalent or multivalent antibodies or antibody fragments, the binding domains can be different targeting different epitopes of the same antigen or targeting different antigens. Methods of constructing multivalent binding constructs are known in the art. Bispecific and multispecific antibodies are known in the art. Furthermore, a diabody, triabody, or tetrabody can be provided, which is a dimer, trimer, or tetramer of polypeptide chains each comprising a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a multimeric molecule having two, three, or four functional antigen binding sites. Also, bis-scFv fragments, which are small scFv fragments with two different variable domains can be generated to produce bispecific bis-scFv fragments capable of binding two different epitopes. Fab dimers (Fab2) and Fab trimers (Fab3) can be produced using genetic engineering methods to create multispecific constructs based on Fab fragments.

The PD-L1-binding agent also can be an antibody conjugate. In this respect, the PD-L1-binding agent can be a conjugate of (1) an antibody, an alternative scaffold, or fragments thereof, and (2) a protein or non-protein moiety. For example, the PD-L1 binding agent can be conjugated to a peptide, a fluorescent molecule, chemotherapeutic or other cytotoxic payload, immune-activating or immune-suppressive agent.

The PD-L1-binding agent can be, or can be obtained from, a human antibody, a non-human antibody, a humanized antibody, or a chimeric antibody, or corresponding antibody fragments. A “chimeric” antibody is an antibody or fragment thereof typically comprising human constant regions and non-human variable regions. A “humanized” antibody is a monoclonal antibody typically comprising a human antibody scaffold but with non-human origin amino acids or sequences in at least one CDR (e.g., 1, 2, 3, 4, 5, or all six CDRs).

### PD-L1-Binding Agents—Type A

Provided herein are PD-L1 binding agents comprising an immunoglobulin heavy chain variable region polypeptide and an immunoglobulin light chain variable region polypeptide. In some embodiments, the PD-L1 binding agents (Type A) comprise an immunoglobulin heavy chain variable region of any one of SEQ ID NOs: 223-264, or at least the CDRs thereof, and an immunoglobulin light chain variable region of any one of SEQ ID NOs: 265-306 or at least the CDRs thereof. In other embodiments, the PD-L1 binding agents (Type A) comprise an immunoglobulin heavy chain variable region polypeptide with an amino acid sequence that is at least 90% identical to any one of SEQ ID NOs: 223-264, and an immunoglobulin light chain variable region polypeptide with an amino acid sequence that is at least 90% identical to any one of SEQ ID NOs: 265-306. In yet other embodiments, the PD-L1 binding agent (Type A), the immunoglobulin heavy chain variable region polypeptide comprises a complementarity determining region 1 (HCDR1) comprising any one of SEQ ID NOs: 1-23, a complementarity determining region 2 (HCDR2) comprising any one of SEQ ID NOs: 24-57, and a complementarity determining region 3 (HCDR3) comprising any one of SEQ ID NOs: 58-95; and/or the immunoglobulin light chain variable region polypeptide comprises a complementarity determining region 1 (LCDR1) comprising any one of SEQ ID NOs: 96-128, a complementarity determining region 2 (LCDR2) comprising any one of SEQ ID NOs: 129-151, and a complementarity determining region 3 (LCDR3) comprising any one of SEQ ID NOs: 152-155. Also provided are nucleic acids encoding the PD-L1 binding agents, or the individual heavy and light chains thereof; vectors and cells comprising the nucleic acids; and compositions comprising the binding agents or nucleic acids.

Furthermore, in some embodiments, the PD-L1 binding agents (Type A) provided herein cause cellular internalization of PD-L1 or the PD-L1/PD-L1 binding agent complex upon binding to PD-L1 on the cell surface. Without wishing to be bound by any particular theory or mechanism of action, it is believed that the PD-L1 binding agents according to this embodiment cause PD-L1 internalization upon binding, and remain bound to PD-L1 during internalization resulting in internalization of the binding agent along with PD-L1. Cellular internalization of PD-L1 and bound PD-L1 binding agent can be determined by any suitable method, such as assaying for persistence on the cell surface and/or detection of internalized antibodies. In some embodiments, the PD-L1 binding agent internalizes strongly enough that at least about 25% (e.g., at least about 35%, at least about 50%, at least about 75%, or at least about 90%) of the PD-L1 binding agent that binds PD-L1 on the cell surface is internalized (e.g., using a surface persistence assay, about 75% or less, about 65% or less, about 50% or less, about 25% or less or about 10% or less of PD-L1 binding agent molecules bound to PD-L1 on the cell surface at the beginning of the assay remain bound at the end of the assay).

In an embodiment, the PD-L1 binding agent (Type A) comprises an immunoglobulin heavy chain variable region of any one of SEQ ID NOs: 223-264, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 223-264, or at least the CDRs thereof, and/or an immunoglobulin light chain variable region of any one of SEQ ID NOs: 265-306, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 265-306, or at least the CDRs thereof.

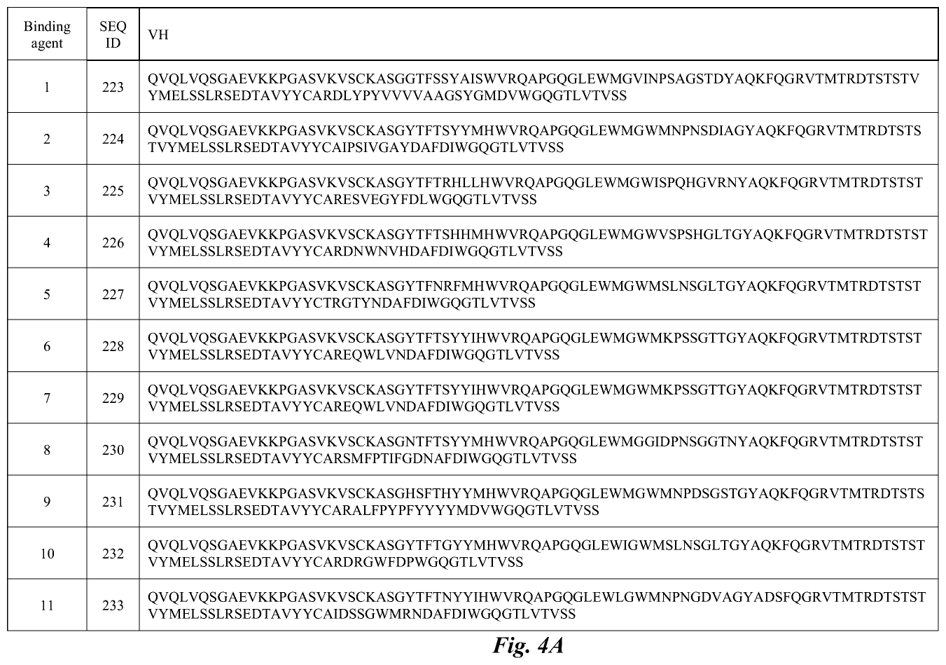

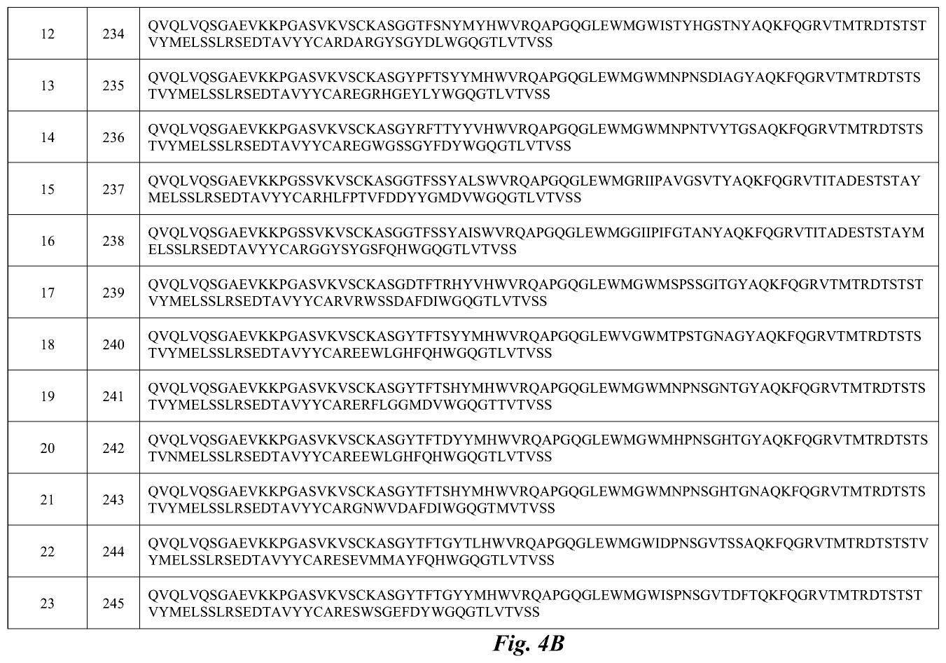

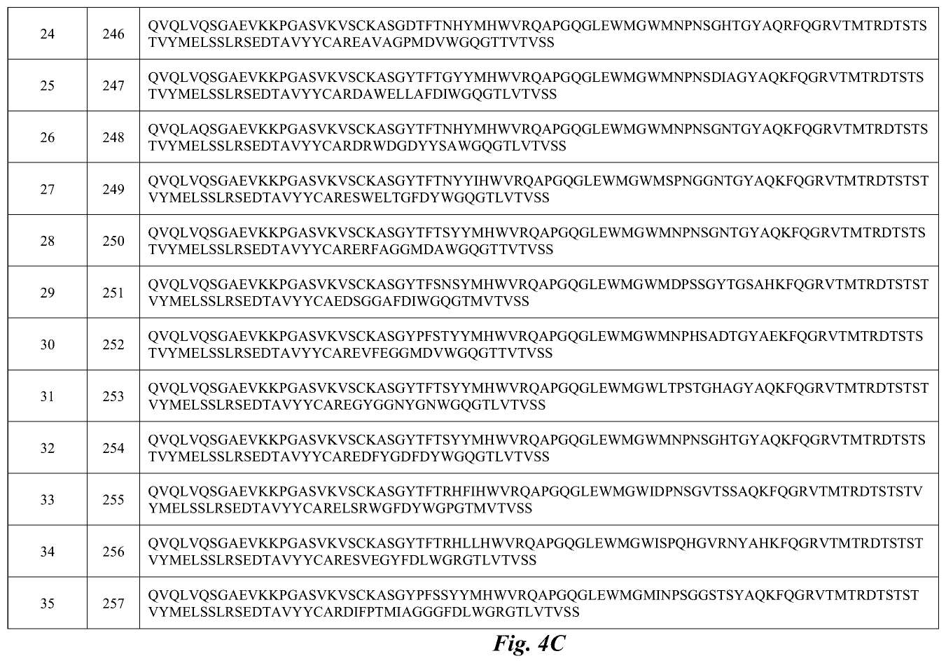

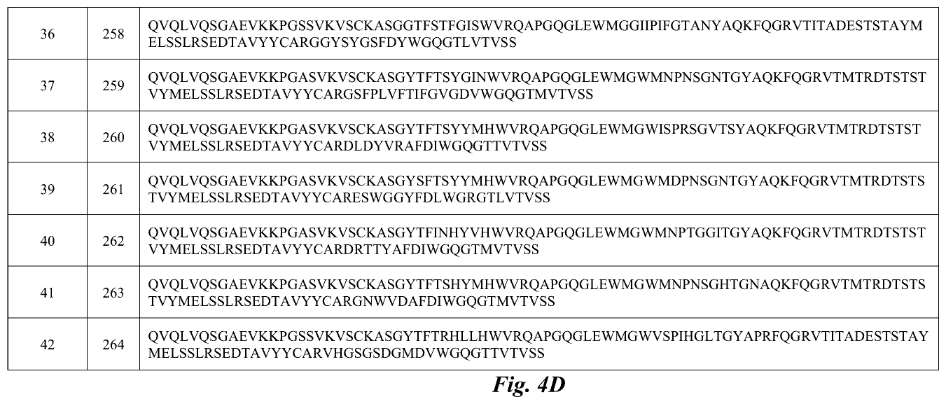

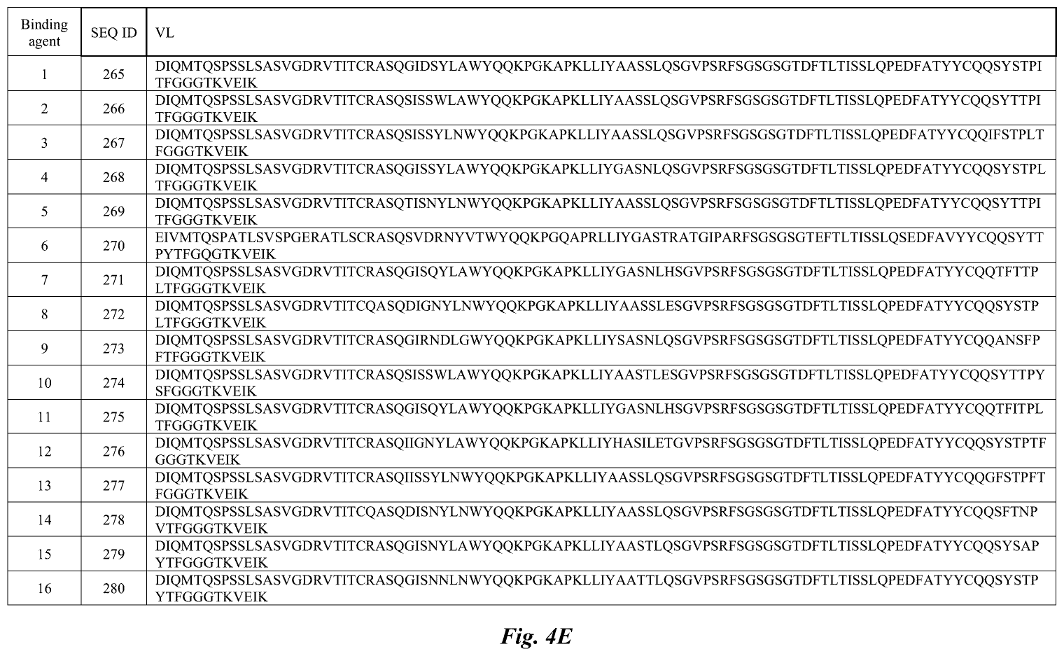

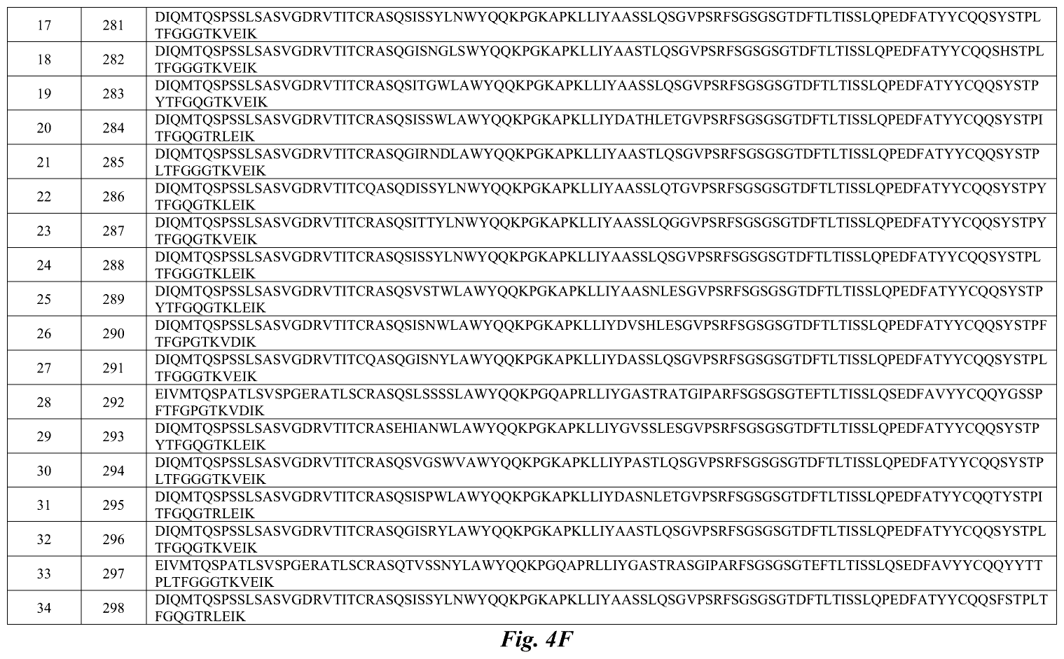

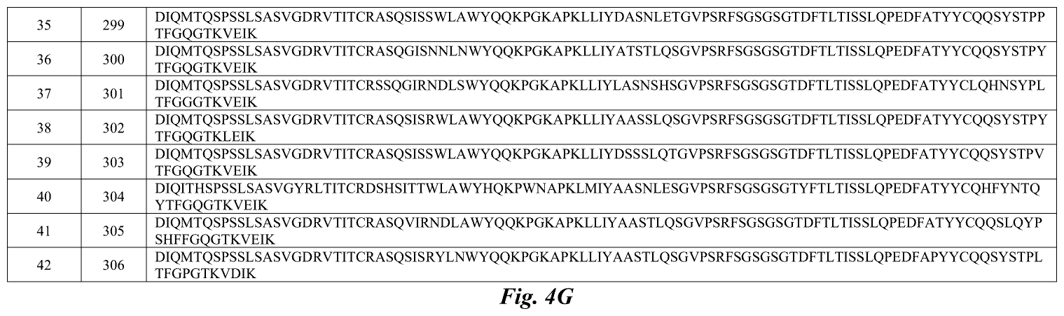

By way of further illustration, the PD-L1 binding agent (Type A) can comprise:
(1) an immunoglobulin heavy chain variable region of SEQ ID NO: 223, or at least the CDRs thereof, and/or an immunoglobulin light chain variable region of SEQ ID NO: 265, or at least the CDRs thereof;
(2) an immunoglobulin heavy chain variable region of SEQ ID NO: 224, or at least the CDRs thereof, and/or an immunoglobulin light chain variable region of SEQ ID NO: 266, or at least the CDRs thereof;
(3) an immunoglobulin heavy chain variable region of SEQ ID NO: 225, or at least the CDRs thereof, and/or an immunoglobulin light chain variable region of SEQ ID NO: 267, or at least the CDRs thereof;
(4) an immunoglobulin heavy chain variable region of SEQ ID NO: 226, or at least the CDRs thereof, and/or an immunoglobulin light chain variable region of SEQ ID NO: 268, or at least the CDRs thereof;
(5) an immunoglobulin heavy chain variable region of SEQ ID NO: 227, or at least the CDRs thereof, and/or an immunoglobulin light chain variable region of SEQ ID NO: 269, or at least the CDRs thereof;
(6) an immunoglobulin heavy chain variable region of SEQ ID NO: 228, or at least the CDRs thereof, and/or an immunoglobulin light chain variable region of SEQ ID NO: 270, or at least the CDRs thereof;
(7) an immunoglobulin heavy chain variable region of SEQ ID NO: 229, or at least the CDRs thereof, and/or an immunoglobulin light chain variable region of SEQ ID NO: 271, or at least the CDRs thereof;
(8) an immunoglobulin heavy chain variable region of SEQ ID NO: 230, or at least the CDRs thereof, and/or an immunoglobulin light chain variable region of SEQ ID NO: 272, or at least the CDRs thereof;
(9) an immunoglobulin heavy chain variable region of SEQ ID NO: 231, or at least the CDRs thereof, and/or an immunoglobulin light chain variable region of SEQ ID NO: 273, or at least the CDRs thereof;
(10) an immunoglobulin heavy chain variable region of SEQ ID NO: 232, or at least the CDRs thereof, and/or an immunoglobulin light chain variable region of SEQ ID NO: 274, or at least the CDRs thereof;
(11) an immunoglobulin heavy chain variable region of SEQ ID NO: 233, or at least the CDRs thereof, and/or an immunoglobulin light chain variable region of SEQ ID NO: 275, or at least the CDRs thereof;
(12) an immunoglobulin heavy chain variable region of SEQ ID NO: 234, or at least the CDRs thereof, and/or an immunoglobulin light chain variable region of SEQ ID NO: 276, or at least the CDRs thereof;
(13) an immunoglobulin heavy chain variable region of SEQ ID NO: 235, or at least the CDRs thereof, and/or an immunoglobulin light chain variable region of SEQ ID NO: 277, or at least the CDRs thereof;
(14) an immunoglobulin heavy chain variable region of SEQ ID NO: 236, or at least the CDRs thereof, and/or an immunoglobulin light chain variable region of SEQ ID NO: 278, or at least the CDRs thereof;
(15) an immunoglobulin heavy chain variable region of SEQ ID NO: 237, or at least the CDRs thereof, and/or an immunoglobulin light chain variable region of SEQ ID NO: 279, or at least the CDRs thereof;
(16) an immunoglobulin heavy chain variable region of SEQ ID NO: 238, or at least the CDRs thereof, and/or an immunoglobulin light chain variable region of SEQ ID NO: 280, or at least the CDRs thereof;
(17) an immunoglobulin heavy chain variable region of SEQ ID NO: 239, or at least the CDRs thereof, and/or an immunoglobulin light chain variable region of SEQ ID NO: 281, or at least the CDRs thereof;
(18) an immunoglobulin heavy chain variable region of SEQ ID NO: 240, or at least the CDRs thereof, and/or an immunoglobulin light chain variable region of SEQ ID NO: 282, or at least the CDRs thereof;
(19) an immunoglobulin heavy chain variable region of SEQ ID NO: 241, or at least the CDRs thereof, and/or an immunoglobulin light chain variable region of SEQ ID NO: 283, or at least the CDRs thereof;
(20) an immunoglobulin heavy chain variable region of SEQ ID NO: 242, or at least the CDRs thereof, and/or an immunoglobulin light chain variable region of SEQ ID NO: 284, or at least the CDRs thereof;
(21) an immunoglobulin heavy chain variable region of SEQ ID NO: 243, or at least the CDRs thereof, and/or an immunoglobulin light chain variable region of SEQ ID NO: 285, or at least the CDRs thereof;
(22) an immunoglobulin heavy chain variable region of SEQ ID NO: 244, or at least the CDRs thereof, and/or an immunoglobulin light chain variable region of SEQ ID NO: 286, or at least the CDRs thereof;
(23) an immunoglobulin heavy chain variable region of SEQ ID NO: 245, or at least the CDRs thereof, and/or an immunoglobulin light chain variable region of SEQ ID NO: 287, or at least the CDRs thereof;
(24) an immunoglobulin heavy chain variable region of SEQ ID NO: 246, or at least the CDRs thereof, and/or an immunoglobulin light chain variable region of SEQ ID NO: 288, or at least the CDRs thereof;
(25) an immunoglobulin heavy chain variable region of SEQ ID NO: 247, or at least the CDRs thereof, and/or an immunoglobulin light chain variable region of SEQ ID NO: 289, or at least the CDRs thereof;
(26) an immunoglobulin heavy chain variable region of SEQ ID NO: 248, or at least the CDRs thereof, and/or an immunoglobulin light chain variable region of SEQ ID NO: 290, or at least the CDRs thereof;
(27) an immunoglobulin heavy chain variable region of SEQ ID NO: 249, or at least the CDRs thereof, and/or an immunoglobulin light chain variable region of SEQ ID NO: 291, or at least the CDRs thereof;
(28) an immunoglobulin heavy chain variable region of SEQ ID NO: 250, or at least the CDRs thereof, and/or an immunoglobulin light chain variable region of SEQ ID NO: 292, or at least the CDRs thereof;
(29) an immunoglobulin heavy chain variable region of SEQ ID NO: 251, or at least the CDRs thereof, and/or an immunoglobulin light chain variable region of SEQ ID NO: 293, or at least the CDRs thereof;
(30) an immunoglobulin heavy chain variable region of SEQ ID NO: 252, or at least the CDRs thereof, and/or an immunoglobulin light chain variable region of SEQ ID NO: 294, or at least the CDRs thereof;
(31) an immunoglobulin heavy chain variable region of SEQ ID NO: 253, or at least the CDRs thereof, and/or an immunoglobulin light chain variable region of SEQ ID NO: 295, or at least the CDRs thereof;
(32) an immunoglobulin heavy chain variable region of SEQ ID NO: 254, or at least the CDRs thereof, and/or an immunoglobulin light chain variable region of SEQ ID NO: 296, or at least the CDRs thereof;
(33) an immunoglobulin heavy chain variable region of SEQ ID NO: 255, or at least the CDRs thereof, and/or an immunoglobulin light chain variable region of SEQ ID NO: 297, or at least the CDRs thereof;
(34) an immunoglobulin heavy chain variable region of SEQ ID NO: 256, or at least the CDRs thereof, and/or an immunoglobulin light chain variable region of SEQ ID NO: 298, or at least the CDRs thereof;
(35) an immunoglobulin heavy chain variable region of SEQ ID NO: 257, or at least the CDRs thereof, and/or an immunoglobulin light chain variable region of SEQ ID NO: 299, or at least the CDRs thereof;
(36) an immunoglobulin heavy chain variable region of SEQ ID NO: 258, or at least the CDRs thereof, and/or an immunoglobulin light chain variable region of SEQ ID NO: 300, or at least the CDRs thereof;
(37) an immunoglobulin heavy chain variable region of SEQ ID NO: 259, or at least the CDRs thereof, and/or an immunoglobulin light chain variable region of SEQ ID NO: 301, or at least the CDRs thereof;
(38) an immunoglobulin heavy chain variable region of SEQ ID NO: 260, or at least the CDRs thereof, and/or an immunoglobulin light chain variable region of SEQ ID NO: 302, or at least the CDRs thereof;
(39) an immunoglobulin heavy chain variable region of SEQ ID NO: 261, or at least the CDRs thereof, and/or an immunoglobulin light chain variable region of SEQ ID NO: 303, or at least the CDRs thereof;
(40) an immunoglobulin heavy chain variable region of SEQ ID NO: 262, or at least the CDRs thereof, and/or an immunoglobulin light chain variable region of SEQ ID NO: 304, or at least the CDRs thereof;
(41) an immunoglobulin heavy chain variable region of SEQ ID NO: 263, or at least the CDRs thereof, and/or an immunoglobulin light chain variable region of SEQ ID NO: 305, or at least the CDRs thereof;
(42) an immunoglobulin heavy chain variable region of SEQ ID NO: 164, or at least the CDRs thereof, and/or an immunoglobulin light chain variable region of SEQ ID NO: 306, or at least the CDRs thereof; and/or
(43) an immunoglobulin heavy chain variable region of FIGS. 4A-D and/or an immunoglobulin light chain variable region of FIGS. 4E-G, or at least the CDRs thereof.

The CDRs of a given heavy or light chain Ig sequence can be determined in accordance with any of the various known Ig numbering schemes (e.g., Kabat, Chothia, Martin (Enhanced Chothia), IGMT, AbM). In certain embodiments, the PD-L1 binding agent (Type A) comprises one or more of the following CDRs:
a HCDR1 comprising or consisting of any one of SEQ ID NOs: 1-23 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 1-23;
a HCDR2 comprising or consisting of any one of SEQ ID NOs: 24-57 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 24-57; and
a HCDR3 comprising or consisting of any one of SEQ ID NOs: 58-95 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 58-95; and/or the immunoglobulin light chain polypeptide comprises
a LCDR1 comprising or consisting of any one of SEQ ID NOs: 96-128 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 96-128;
a LCDR2 comprising or consisting of any one of SEQ ID NOs: 129-151 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 129-151; and
a LCDR3 comprising or consisting of any one of SEQ ID NOs: 152-155 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 152-155.

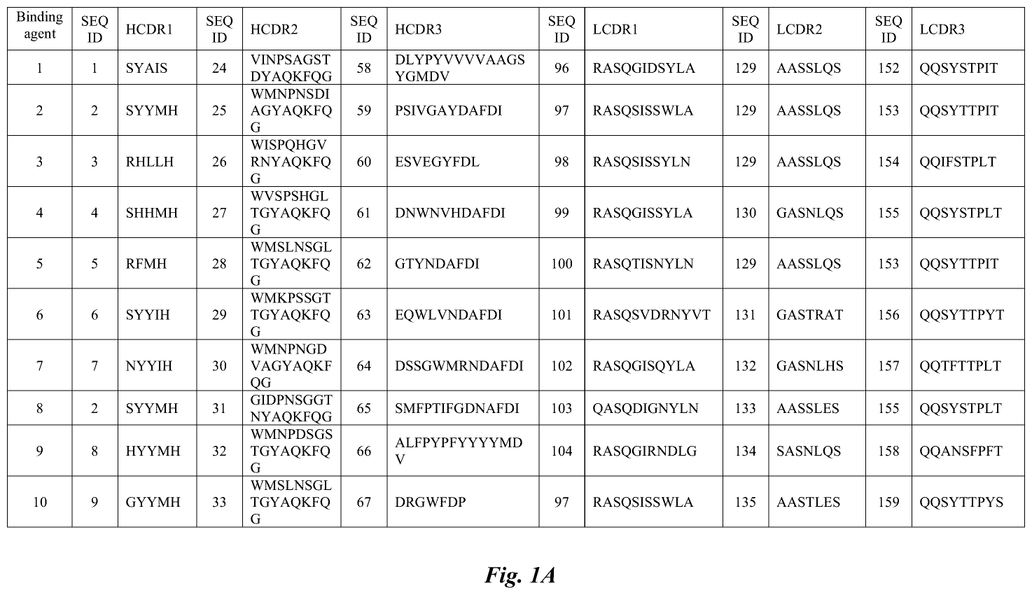

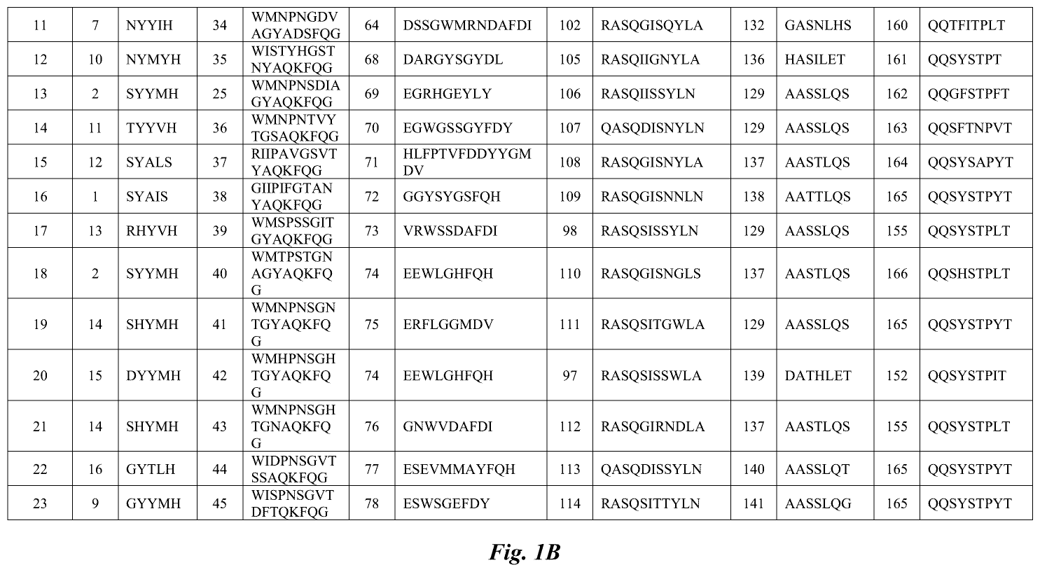

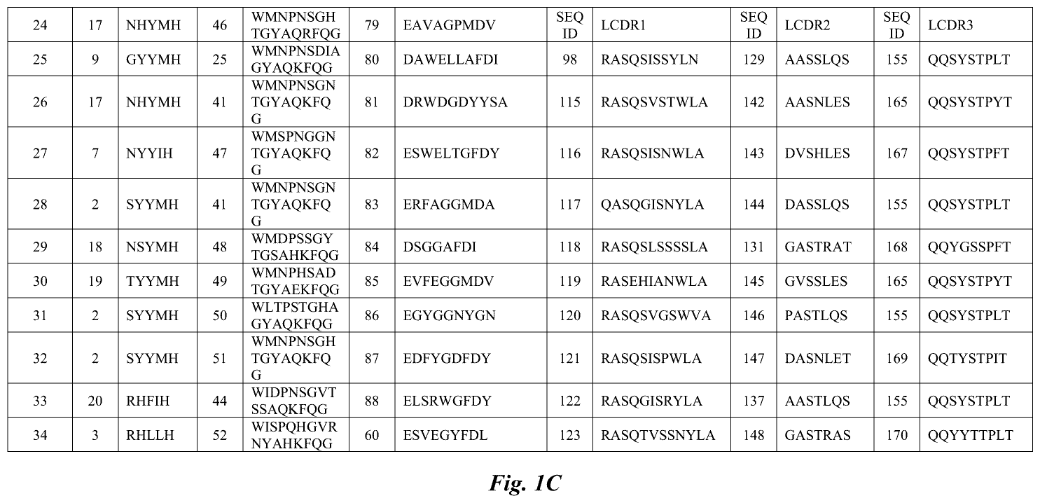

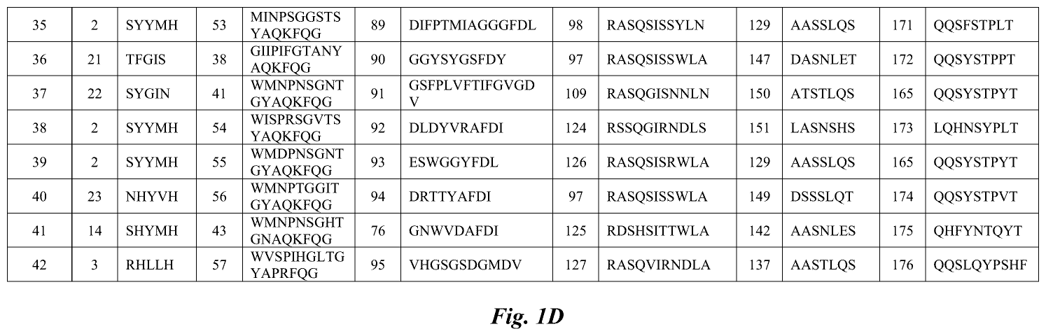

In particular embodiments, the binding agent (Type A) comprises an immunoglobulin heavy chain polypeptide and an immunoglobulin light chain polypeptide, wherein:
(1) the immunoglobulin heavy chain polypeptide comprises a HCDR1 comprising or consisting of SEQ ID NO: 1, a HCDR2 comprising or consisting of SEQ ID NO: 24, and a HCDR3 comprising or consisting of SEQ ID NO: 58; and/or the immunoglobulin light chain polypeptide comprises a LCDR1 comprising or consisting of SEQ ID NO: 96, a LCDR2 comprising or consisting of SEQ ID NO: 129, and a LCDR3 comprising or consisting of SEQ ID NO: 152;
(2) the immunoglobulin heavy chain polypeptide comprises a HCDR1 comprising or consisting of SEQ ID NO: 2, a HCDR2 comprising or consisting of SEQ ID NO: 25, and a HCDR3 comprising or consisting of SEQ ID NO: 59; and/or the immunoglobulin light chain polypeptide comprises a LCDR1 comprising or consisting of SEQ ID NO: 97, a LCDR2 comprising or consisting of SEQ ID NO: 129, and a LCDR3 comprising or consisting of SEQ ID NO: 153;
(3) the immunoglobulin heavy chain polypeptide comprises a HCDR1 comprising or consisting of SEQ ID NO: 3, a HCDR2 comprising or consisting of SEQ ID NO: 26, and a HCDR3 comprising or consisting of SEQ ID NO: 60; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 154;
(4) the immunoglobulin heavy chain polypeptide comprises a HCDR1 comprising or consisting of SEQ ID NO: 4, a HCDR2 comprising or consisting of SEQ ID NO: 27, and a HCDR3 comprising or consisting of SEQ ID NO: 61; and/or the immunoglobulin light chain polypeptide comprises a LCDR1 comprising or consisting of SEQ ID NO: 99, a LCDR2 comprising or consisting of SEQ ID NO: 130, and a LCDR3 comprising or consisting of SEQ ID NO: 155;
(5) the immunoglobulin heavy chain polypeptide comprises a HCDR1 comprising or consisting of SEQ ID NO: 5, a HCDR2 comprising or consisting of SEQ ID NO: 28, and a HCDR3 comprising or consisting of SEQ ID NO: 62; and/or the immunoglobulin light chain polypeptide comprises a LCDR1 comprising or consisting of SEQ ID NO: 100, a LCDR2 comprising or consisting of SEQ ID NO: 129, and a LCDR3 comprising or consisting of SEQ ID NO: 153;
(6) the immunoglobulin heavy chain polypeptide comprises a HCDR1 comprising or consisting of SEQ ID NO: 6, a HCDR2 comprising or consisting of SEQ ID NO: 29, and a HCDR3 comprising or consisting of SEQ ID NO: 63; and/or the immunoglobulin light chain polypeptide comprises a LCDR1 comprising or consisting of SEQ ID NO: 101, a LCDR2 comprising or consisting of SEQ ID NO: 131, and a LCDR3 comprising or consisting of SEQ ID NO: 156;
(7) the immunoglobulin heavy chain polypeptide comprises a HCDR1 comprising or consisting of SEQ ID NO: 7, a HCDR2 comprising or consisting of SEQ ID NO: 30, and a HCDR3 comprising or consisting of SEQ ID NO: 64; and/or the immunoglobulin light chain polypeptide comprises a LCDR1 comprising or consisting of SEQ ID NO: 102, a LCDR2 comprising or consisting of SEQ ID NO: 132, and a LCDR3 comprising or consisting of SEQ ID NO: 157;
(8) the immunoglobulin heavy chain polypeptide comprises a HCDR1 comprising or consisting of SEQ ID NO: 2, a HCDR2 comprising or consisting of SEQ ID NO: 31, and a HCDR3 comprising or consisting of SEQ ID NO: 65; and/or the immunoglobulin light chain polypeptide comprises a LCDR1 comprising or consisting of SEQ ID NO: 103, a LCDR2 comprising or consisting of SEQ ID NO: 133, and a LCDR3 comprising or consisting of SEQ ID NO: 155;
(9) the immunoglobulin heavy chain polypeptide comprises a HCDR1 comprising or consisting of SEQ ID NO: 8, a HCDR2 comprising or consisting of SEQ ID NO: 32, and a HCDR3 comprising or consisting of SEQ ID NO: 66; and/or the immunoglobulin light chain polypeptide comprises a LCDR1 comprising or consisting of SEQ ID NO: 104, a LCDR2 comprising or consisting of SEQ ID NO: 134, and a LCDR3 comprising or consisting of SEQ ID NO: 158;
(10) the immunoglobulin heavy chain polypeptide comprises a HCDR1 comprising or consisting of SEQ ID NO: 9, a HCDR2 comprising or consisting of SEQ ID NO: 33, and a HCDR3 comprising or consisting of SEQ ID NO: 67; and/or the immunoglobulin light chain polypeptide comprises a LCDR1 comprising or consisting of SEQ ID NO: 97, a LCDR2 comprising or consisting of SEQ ID NO: 135, and a LCDR3 comprising or consisting of SEQ ID NO: 159;
(11) the immunoglobulin heavy chain polypeptide comprises a HCDR1 comprising or consisting of SEQ ID NO: 7, a HCDR2 comprising or consisting of SEQ ID NO: 34, and a HCDR3 comprising or consisting of SEQ ID NO: 64; and/or the immunoglobulin light chain polypeptide comprises a LCDR1 comprising or consisting of SEQ ID NO: 102, a LCDR2 comprising or consisting of SEQ ID NO: 132, and a LCDR3 comprising or consisting of SEQ ID NO: 160;
(12) the immunoglobulin heavy chain polypeptide comprises a HCDR1 comprising or consisting of SEQ ID NO: 10, a HCDR2 comprising or consisting of SEQ ID NO: 35, and a HCDR3 comprising or consisting of SEQ ID NO: 68; and/or the immunoglobulin light chain polypeptide comprises a LCDR1 comprising or consisting of SEQ ID NO: 105, a LCDR2 comprising or consisting of SEQ ID NO: 136, and a LCDR3 comprising or consisting of SEQ ID NO: 161;
(13) the immunoglobulin heavy chain polypeptide comprises a HCDR1 comprising or consisting of SEQ ID NO: 2, a HCDR2 comprising or consisting of SEQ ID NO: 25, and a HCDR3 comprising or consisting of SEQ ID NO: 69; and/or the immunoglobulin light chain polypeptide comprises a LCDR1 comprising or consisting of SEQ ID NO: 106, a LCDR2 comprising or consisting of SEQ ID NO: 129, and a LCDR3 comprising or consisting of SEQ ID NO: 162;
(14) the immunoglobulin heavy chain polypeptide comprises a HCDR1 comprising or consisting of SEQ ID NO: 11, a HCDR2 comprising or consisting of SEQ ID NO: 36, and a HCDR3 comprising or consisting of SEQ ID NO: 70; and/or the immunoglobulin light chain polypeptide comprises a LCDR1 comprising or consisting of SEQ ID NO: 107, a LCDR2 comprising or consisting of SEQ ID NO: 129, and a LCDR3 comprising or consisting of SEQ ID NO: 163;
(15) the immunoglobulin heavy chain polypeptide comprises a HCDR1 comprising or consisting of SEQ ID NO: 12, a HCDR2 comprising or consisting of SEQ ID NO: 37, and a HCDR3 comprising or consisting of SEQ ID NO: 71; and/or the immunoglobulin light chain polypeptide comprises a LCDR1 comprising or consisting of SEQ ID NO: 108, a LCDR2 comprising or consisting of SEQ ID NO: 137, and a LCDR3 comprising or consisting of SEQ ID NO: 164;
(16) the immunoglobulin heavy chain polypeptide comprises a HCDR1 comprising or consisting of SEQ ID NO: 1, a HCDR2 comprising or consisting of SEQ ID NO: 38, and a HCDR3 comprising or consisting of SEQ ID NO: 72; and/or the immunoglobulin light chain polypeptide comprises a LCDR1 comprising or consisting of SEQ ID NO: 109, a LCDR2 comprising or consisting of SEQ ID NO: 138, and a LCDR3 comprising or consisting of SEQ ID NO: 165;
(17) the immunoglobulin heavy chain polypeptide comprises a HCDR1 comprising or consisting of SEQ ID NO: 13, a HCDR2 comprising or consisting of SEQ ID NO: 39, and a HCDR3 comprising or consisting of SEQ ID NO: 73; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 155;
(18) the immunoglobulin heavy chain polypeptide comprises a HCDR1 comprising or consisting of SEQ ID NO: 2, a HCDR2 comprising or consisting of SEQ ID NO: 40, and a HCDR3 comprising or consisting of SEQ ID NO: 74; and/or the immunoglobulin light chain polypeptide comprises a LCDR1 comprising or consisting of SEQ ID NO: 110, a LCDR2 comprising or consisting of SEQ ID NO: 137, and a LCDR3 comprising or consisting of SEQ ID NO: 166;
(19) the immunoglobulin heavy chain polypeptide comprises a HCDR1 comprising or consisting of SEQ ID NO: 14, a HCDR2 comprising or consisting of SEQ ID NO: 41, and a HCDR3 comprising or consisting of SEQ ID NO: 75; and/or the immunoglobulin light chain polypeptide comprises a LCDR1 comprising or consisting of SEQ ID NO: 111, a LCDR2 comprising or consisting of SEQ ID NO: 129, and a LCDR3 comprising or consisting of SEQ ID NO: 165;
(20) the immunoglobulin heavy chain polypeptide comprises a HCDR1 comprising or consisting of SEQ ID NO: 15, a HCDR2 comprising or consisting of SEQ ID NO: 42, and a HCDR3 comprising or consisting of SEQ ID NO: 74; and/or the immunoglobulin light chain polypeptide comprises a LCDR1 comprising or consisting of SEQ ID NO: 97, a LCDR2 comprising or consisting of SEQ ID NO: 139, and a LCDR3 comprising or consisting of SEQ ID NO: 152;
(21) the immunoglobulin heavy chain polypeptide comprises a HCDR1 comprising or consisting of SEQ ID NO: 14, a HCDR2 comprising or consisting of SEQ ID NO: 43, and a HCDR3 comprising or consisting of SEQ ID NO: 76; and/or the immunoglobulin light chain polypeptide comprises a LCDR1 comprising or consisting of SEQ ID NO: 112, a LCDR2 comprising or consisting of SEQ ID NO: 137, and a LCDR3 comprising or consisting of SEQ ID NO: 155;
(22) the immunoglobulin heavy chain polypeptide comprises a HCDR1 comprising or consisting of SEQ ID NO: 16, a HCDR2 comprising or consisting of SEQ ID NO: 44, and a HCDR3 comprising or consisting of SEQ ID NO: 77; and/or the immunoglobulin light chain polypeptide comprises a LCDR1 comprising or consisting of SEQ ID NO: 113, a LCDR2 comprising or consisting of SEQ ID NO: 140, and a LCDR3 comprising or consisting of SEQ ID NO: 165;
(23) the immunoglobulin heavy chain polypeptide comprises a HCDR1 comprising or consisting of SEQ ID NO: 9, a HCDR2 comprising or consisting of SEQ ID NO: 45, and a HCDR3 comprising or consisting of SEQ ID NO: 78; and/or the immunoglobulin light chain polypeptide comprises a LCDR1 comprising or consisting of SEQ ID NO: 114, a LCDR2 comprising or consisting of SEQ ID NO: 141, and a LCDR3 comprising or consisting of SEQ ID NO: 165;
(24) the immunoglobulin heavy chain polypeptide comprises a HCDR1 comprising or consisting of SEQ ID NO: 17, a HCDR2 comprising or consisting of SEQ ID NO: 46, and a HCDR3 comprising or consisting of SEQ ID NO: 79; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 155;
(25) the immunoglobulin heavy chain polypeptide comprises a HCDR1 comprising or consisting of SEQ ID NO: 9, a HCDR2 comprising or consisting of SEQ ID NO: 25, and a HCDR3 comprising or consisting of SEQ ID NO: 80; and/or the immunoglobulin light chain polypeptide comprises a LCDR1 comprising or consisting of SEQ ID NO: 115, a LCDR2 comprising or consisting of SEQ ID NO: 142, and a LCDR3 comprising or consisting of SEQ ID NO: 165;
(26) the immunoglobulin heavy chain polypeptide comprises a HCDR1 comprising or consisting of SEQ ID NO: 17, a HCDR2 comprising or consisting of SEQ ID NO: 41, and a HCDR3 comprising or consisting of SEQ ID NO: 81; and/or the immunoglobulin light chain polypeptide comprises a LCDR1 comprising or consisting of SEQ ID NO: 116, a LCDR2 comprising or consisting of SEQ ID NO: 143, and a LCDR3 comprising or consisting of SEQ ID NO: 167;
(27) the immunoglobulin heavy chain polypeptide comprises a HCDR1 comprising or consisting of SEQ ID NO: 7, a HCDR2 comprising or consisting of SEQ ID NO: 47, and a HCDR3 comprising or consisting of SEQ ID NO: 82; and/or the immunoglobulin light chain polypeptide comprises a LCDR1 comprising or consisting of SEQ ID NO: 117, a LCDR2 comprising or consisting of SEQ ID NO: 144, and a LCDR3 comprising or consisting of SEQ ID NO: 155;
(28) the immunoglobulin heavy chain polypeptide comprises a HCDR1 comprising or consisting of SEQ ID NO: 2, a HCDR2 comprising or consisting of SEQ ID NO: 41, and a HCDR3 comprising or consisting of SEQ ID NO: 83; and/or the immunoglobulin light chain polypeptide comprises a LCDR1 comprising or consisting of SEQ ID NO: 118, a LCDR2 comprising or consisting of SEQ ID NO: 131, and a LCDR3 comprising or consisting of SEQ ID NO: 168;
(29) the immunoglobulin heavy chain polypeptide comprises a HCDR1 comprising or consisting of SEQ ID NO: 18, a HCDR2 comprising or consisting of SEQ ID NO: 48, and a HCDR3 comprising or consisting of SEQ ID NO: 84; and/or the immunoglobulin light chain polypeptide comprises a LCDR1 comprising or consisting of SEQ ID NO: 119, a LCDR2 comprising or consisting of SEQ ID NO: 145, and a LCDR3 comprising or consisting of SEQ ID NO: 165;
(30) the immunoglobulin heavy chain polypeptide comprises a HCDR1 comprising or consisting of SEQ ID NO: 19, a HCDR2 comprising or consisting of SEQ ID NO: 49, and a HCDR3 comprising or consisting of SEQ ID NO: 85; and/or the immunoglobulin light chain polypeptide comprises a LCDR1 comprising or consisting of SEQ ID NO: 120, a LCDR2 comprising or consisting of SEQ ID NO: 146, and a LCDR3 comprising or consisting of SEQ ID NO: 155;
(31) the immunoglobulin heavy chain polypeptide comprises a HCDR1 comprising or consisting of SEQ ID NO: 2, a HCDR2 comprising or consisting of SEQ ID NO: 50, and a HCDR3 comprising or consisting of SEQ ID NO: 86; and/or the immunoglobulin light chain polypeptide comprises a LCDR1 comprising or consisting of SEQ ID NO: 121, a LCDR2 comprising or consisting of SEQ ID NO: 147, and a LCDR3 comprising or consisting of SEQ ID NO: 169;
(32) the immunoglobulin heavy chain polypeptide comprises a HCDR1 comprising or consisting of SEQ ID NO: 2, a HCDR2 comprising or consisting of SEQ ID NO: 51, and a HCDR3 comprising or consisting of SEQ ID NO: 87; and/or the immunoglobulin light chain polypeptide comprises a LCDR1 comprising or consisting of SEQ ID NO: 122, a LCDR2 comprising or consisting of SEQ ID NO: 137, and a LCDR3 comprising or consisting of SEQ ID NO: 155;
(33) the immunoglobulin heavy chain polypeptide comprises a HCDR1 comprising or consisting of SEQ ID NO: 20, a HCDR2 comprising or consisting of SEQ ID NO: 44, and a HCDR3 comprising or consisting of SEQ ID NO: 88; and/or the immunoglobulin light chain polypeptide comprises a LCDR1 comprising or consisting of SEQ ID NO: 123, a LCDR2 comprising or consisting of SEQ ID NO: 148, and a LCDR3 comprising or consisting of SEQ ID NO: 170;
(34) the immunoglobulin heavy chain polypeptide comprises a HCDR1 comprising or consisting of SEQ ID NO: 3, a HCDR2 comprising or consisting of SEQ ID NO: 52, and a HCDR3 comprising or consisting of SEQ ID NO: 60; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 171;
(35) the immunoglobulin heavy chain polypeptide comprises a HCDR1 comprising or consisting of SEQ ID NO: 2, a HCDR2 comprising or consisting of SEQ ID NO: 53, and a HCDR3 comprising or consisting of SEQ ID NO: 89; and/or the immunoglobulin light chain polypeptide comprises a LCDR1 comprising or consisting of SEQ ID NO: 97, a LCDR2 comprising or consisting of SEQ ID NO: 147, and a LCDR3 comprising or consisting of SEQ ID NO: 172;
(36) the immunoglobulin heavy chain polypeptide comprises a HCDR1 comprising or consisting of SEQ ID NO: 21, a HCDR2 comprising or consisting of SEQ ID NO: 38, and a HCDR3 comprising or consisting of SEQ ID NO: 90; and/or the immunoglobulin light chain polypeptide comprises a LCDR1 comprising or consisting of SEQ ID NO: 109, a LCDR2 comprising or consisting of SEQ ID NO: 150, and a LCDR3 comprising or consisting of SEQ ID NO: 165;
(37) the immunoglobulin heavy chain polypeptide comprises a HCDR1 comprising or consisting of SEQ ID NO: 22, a HCDR2 comprising or consisting of SEQ ID NO: 41, and a HCDR3 comprising or consisting of SEQ ID NO: 91; and/or the immunoglobulin light chain polypeptide comprises a LCDR1 comprising or consisting of SEQ ID NO: 124, a LCDR2 comprising or consisting of SEQ ID NO: 151, and a LCDR3 comprising or consisting of SEQ ID NO: 173;
(38) the immunoglobulin heavy chain polypeptide comprises a HCDR1 comprising or consisting of SEQ ID NO: 2, a HCDR2 comprising or consisting of SEQ ID NO: 54, and a HCDR3 comprising or consisting of SEQ ID NO: 92; and/or the immunoglobulin light chain polypeptide comprises a LCDR1 comprising or consisting of SEQ ID NO: 126, a LCDR2 comprising or consisting of SEQ ID NO: 129, and a LCDR3 comprising or consisting of SEQ ID NO: 165;
(39) the immunoglobulin heavy chain polypeptide comprises a HCDR1 comprising or consisting of SEQ ID NO: 2, a HCDR2 comprising or consisting of SEQ ID NO: 55, and a HCDR3 comprising or consisting of SEQ ID NO: 93; and/or the immunoglobulin light chain polypeptide comprises a LCDR1 comprising or consisting of SEQ ID NO: 97, a LCDR2 comprising or consisting of SEQ ID NO: 149, and a LCDR3 comprising or consisting of SEQ ID NO: 174;
(40) the immunoglobulin heavy chain polypeptide comprises a HCDR1 comprising or consisting of SEQ ID NO: 23, a HCDR2 comprising or consisting of SEQ ID NO: 56, and a HCDR3 comprising or consisting of SEQ ID NO: 94; and/or the immunoglobulin light chain polypeptide comprises a LCDR1 comprising or consisting of SEQ ID NO: 125, a LCDR2 comprising or consisting of SEQ ID NO: 142, and a LCDR3 comprising or consisting of SEQ ID NO: 175;
(41) the immunoglobulin heavy chain polypeptide comprises a HCDR1 comprising or consisting of SEQ ID NO: 14, a HCDR2 comprising or consisting of SEQ ID NO: 43, and a HCDR3 comprising or consisting of SEQ ID NO: 76; and/or the immunoglobulin light chain polypeptide comprises a LCDR1 comprising or consisting of SEQ ID NO: 127, a LCDR2 comprising or consisting of SEQ ID NO: 137, and a LCDR3 comprising or consisting of SEQ ID NO: 176;
(42) the immunoglobulin heavy chain polypeptide comprises a HCDR1 comprising or consisting of SEQ ID NO: 3, a HCDR2 comprising or consisting of SEQ ID NO: 57, and a HCDR3 comprising or consisting of SEQ ID NO: 95; and/or the immunoglobulin light chain polypeptide comprises a LCDR1 comprising or consisting of SEQ ID NO: 128, a LCDR2 comprising or consisting of SEQ ID NO: 137, and a LCDR3 comprising or consisting of SEQ ID NO: 155; and/or
(43) the immunoglobulin heavy chain polypeptide and light chain polypeptide comprises any combination of the CDRs listed in FIGS. 1A-D of PD-L1 Type A binding agents 1-42

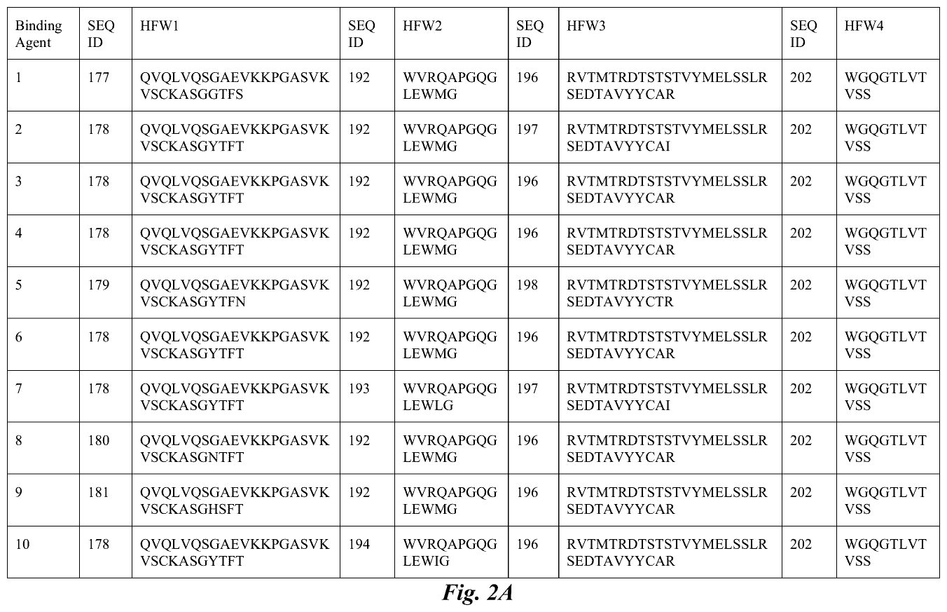

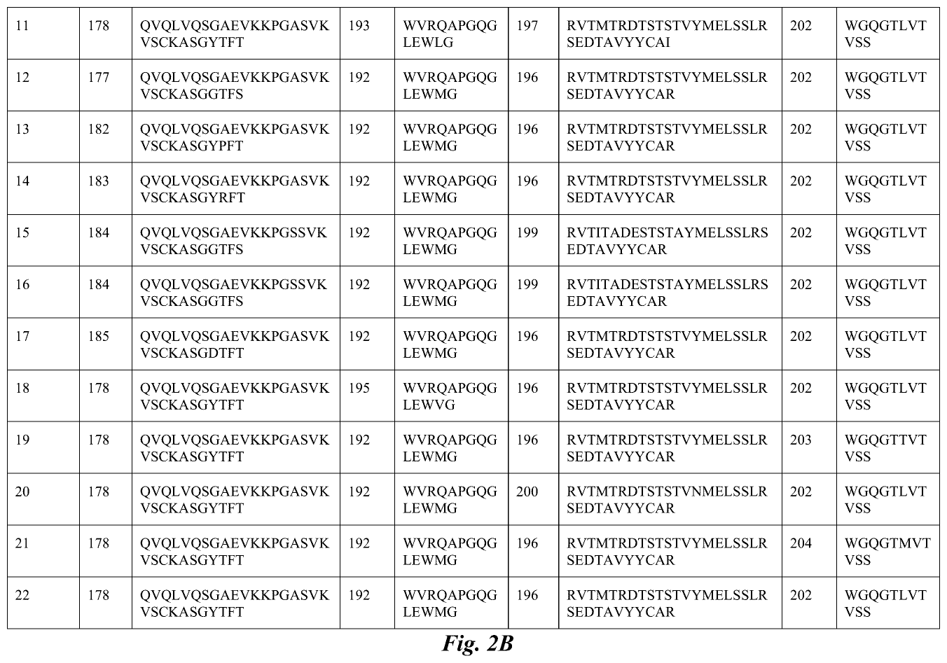

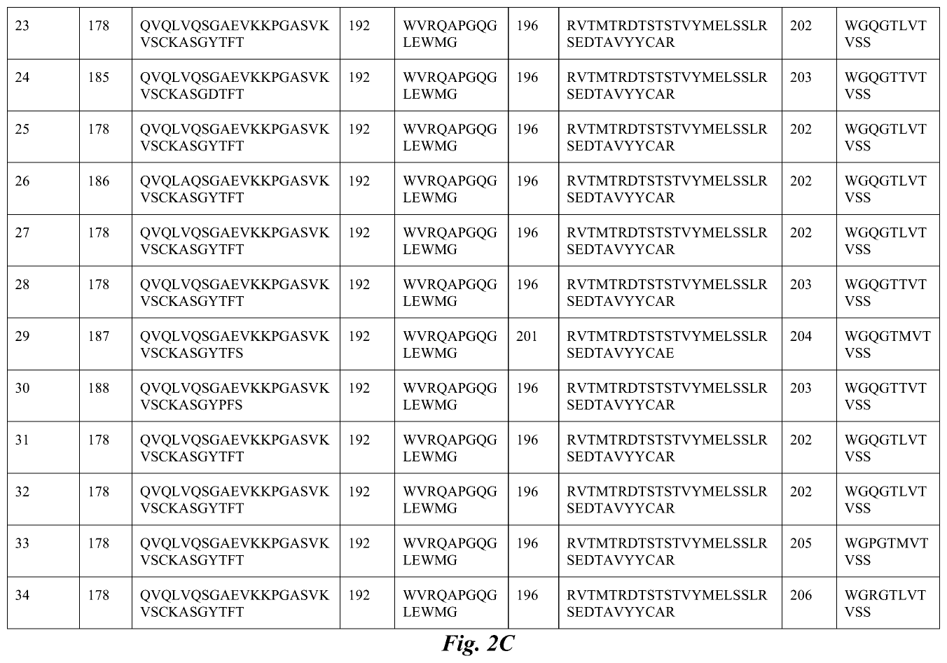

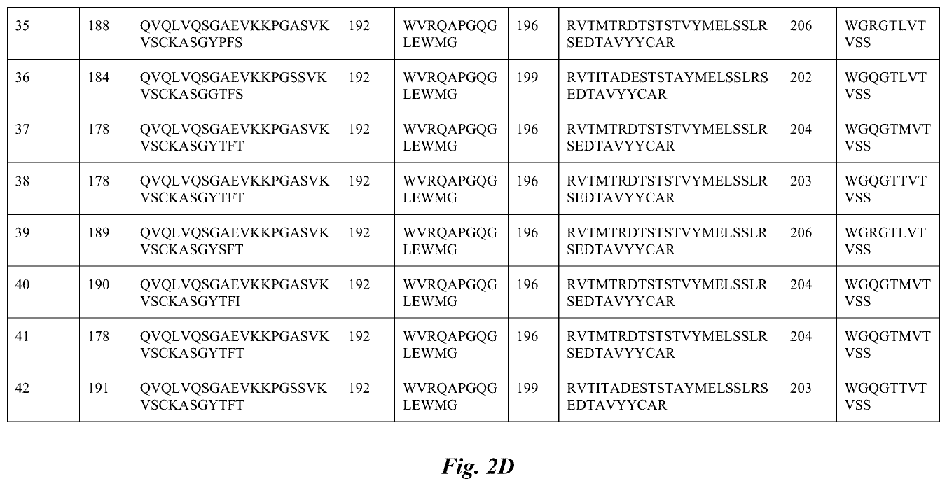

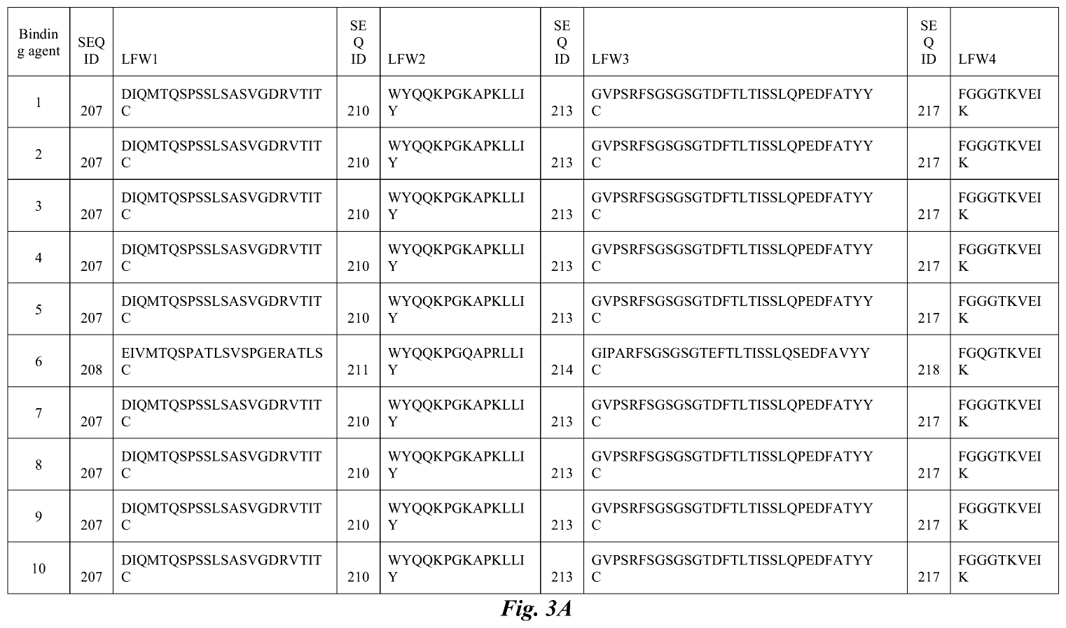

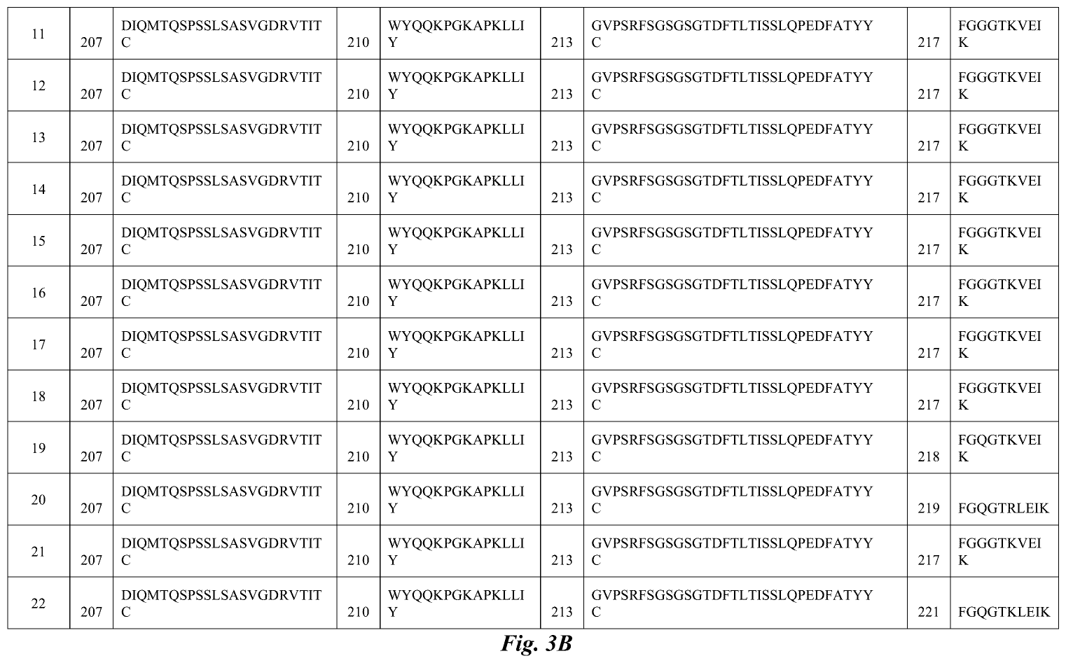

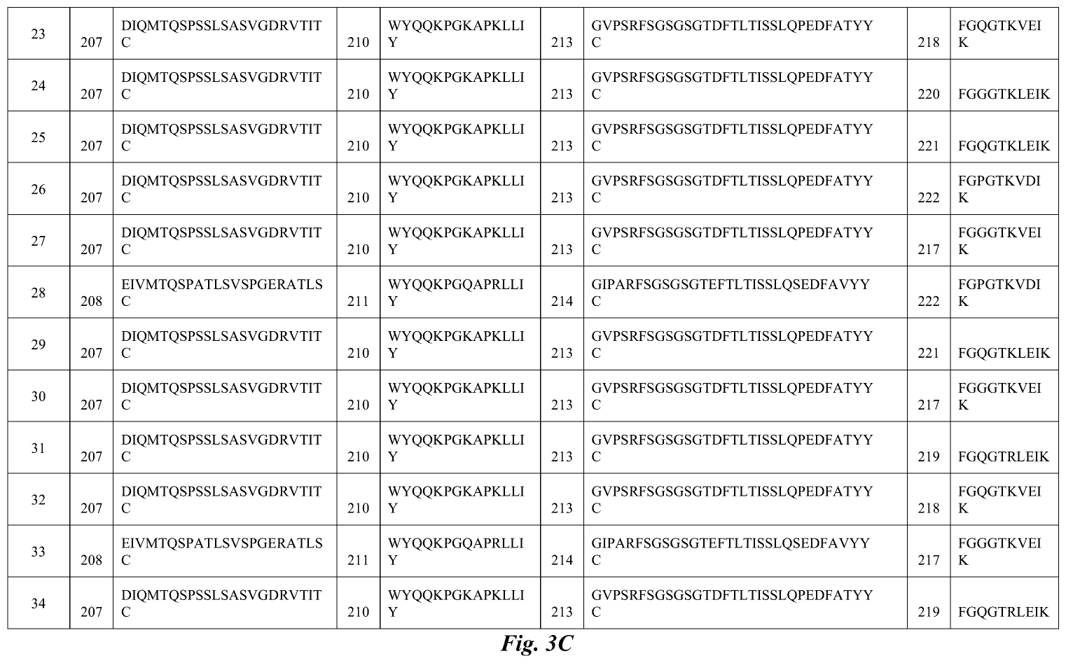

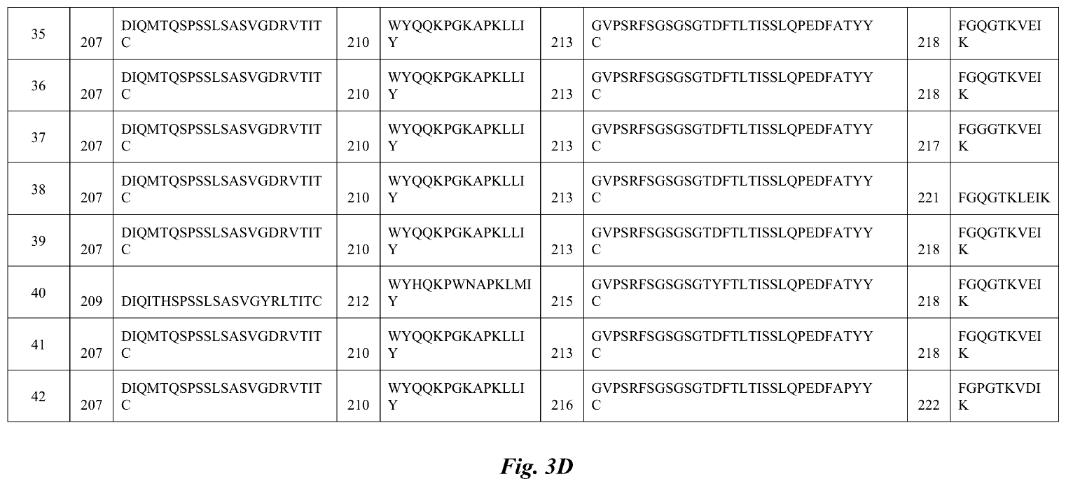

In particular embodiments, the binding agent comprises an immunoglobulin heavy chain polypeptide and an immunoglobulin light chain polypeptide, wherein the immunoglobulin heavy chain polypeptide comprises a first framework region, a second framework region, a third framework region, and/or a fourth framework region; and/or the immunoglobulin light chain polypeptide comprises a first framework region, a second framework region, a third framework region, and/or a fourth framework region; and/or the immunoglobulin heavy chain polypeptide and light chain polypeptide comprises any combination of the framework regions listed in FIGS. 2A-D and FIGS. 3A-D, respectively.

### PD-L1-Binding Agents—Type B

Provided herein are PD-L1 binding agents (Type B) comprising an immunoglobulin heavy chain variable region polypeptide and an immunoglobulin light chain variable region polypeptide. In some embodiments, the PD-L1 binding agents (Type B) comprise an immunoglobulin heavy chain variable region of any one of SEQ ID NOs: 430-450, or at least the CDRs thereof, and an immunoglobulin light chain variable region of any one of SEQ ID NOs: 451-471, or at least the CDRs thereof. In other embodiments, the PD-L1 binding agents comprise an immunoglobulin heavy chain variable region polypeptide with an amino acid sequence that is at least 90% identical to any one of SEQ ID NOs: 430-450, and an immunoglobulin light chain variable region polypeptide with an amino acid sequence that is at least 90% identical to any one of SEQ ID NOs: 451-471. In yet other embodiments, the PD-L1 binding agent, the immunoglobulin heavy chain variable region polypeptide comprises a complementarity determining region 1 (HCDR1) comprising any one of SEQ ID NOs: 308-321, a complementarity determining region 2 (HCDR2) comprising any one of SEQ ID NOs: 322-338, and a complementarity determining region 3 (HCDR3) comprising any one of SEQ ID NOs: 339-359; and/or the immunoglobulin light chain variable region polypeptide comprises a complementarity determining region 1 (LCDR1) comprising any one of SEQ ID NOs: 360-374, a complementarity determining region 2 (LCDR2) comprising any one of SEQ ID NOs: 131 and 375-386, and a complementarity determining region 3 (LCDR3) comprising any one of SEQ ID NOs: 387-398. Also provided are nucleic acids encoding the PD-L1 binding agents, or the individual heavy and light chains thereof; vectors and cells comprising the nucleic acids; and compositions comprising the binding agents or nucleic acids.

In an embodiment, the PD-L1 binding agent (Type B) comprises an immunoglobulin heavy chain variable region of any one of SEQ ID NOs: 430-450, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 430-450, or at least the CDRs thereof, and/or an immunoglobulin light chain variable region of any one of SEQ ID NOs: 451-471, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 451-471, or at least the CDRs thereof.

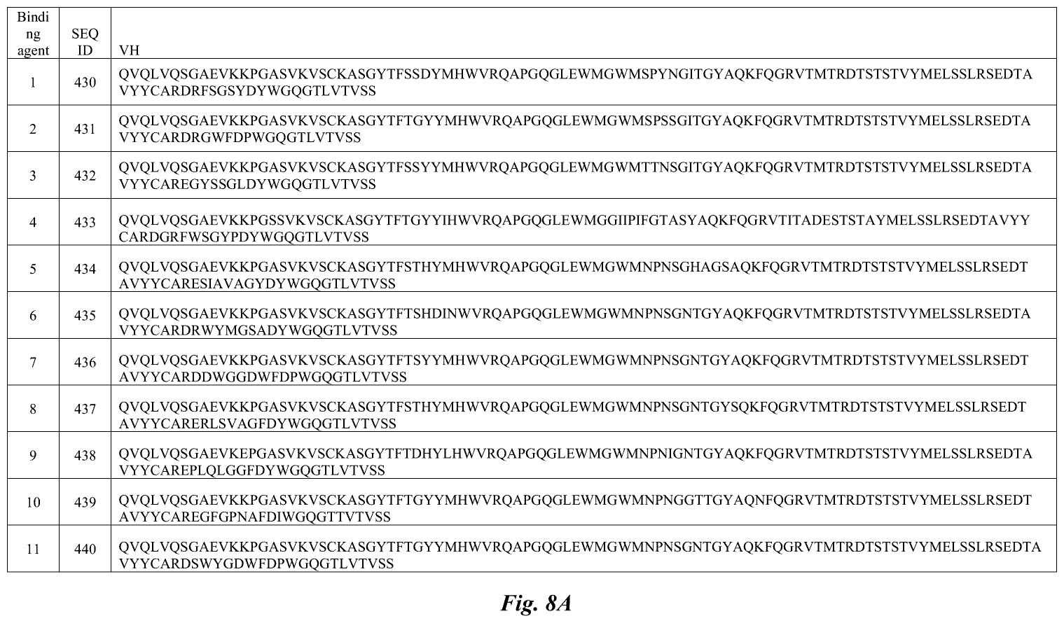

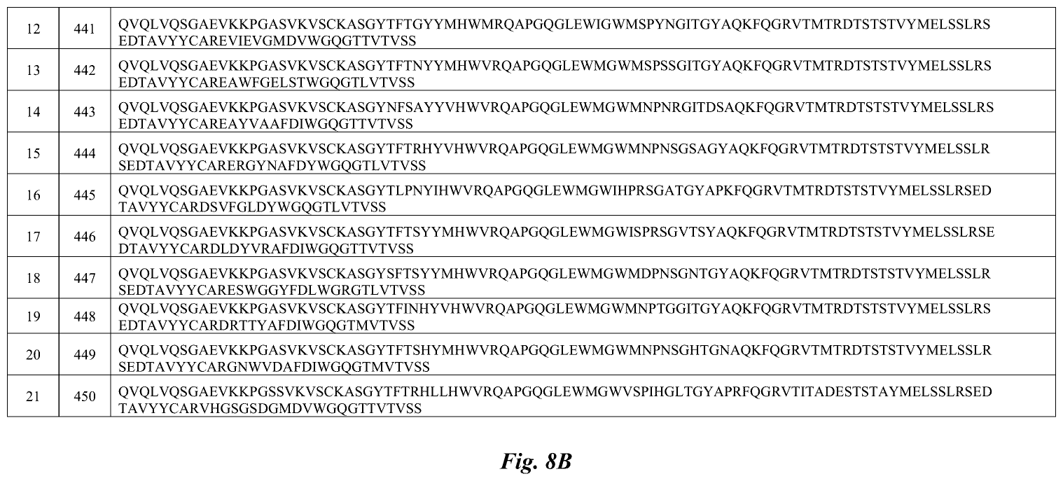

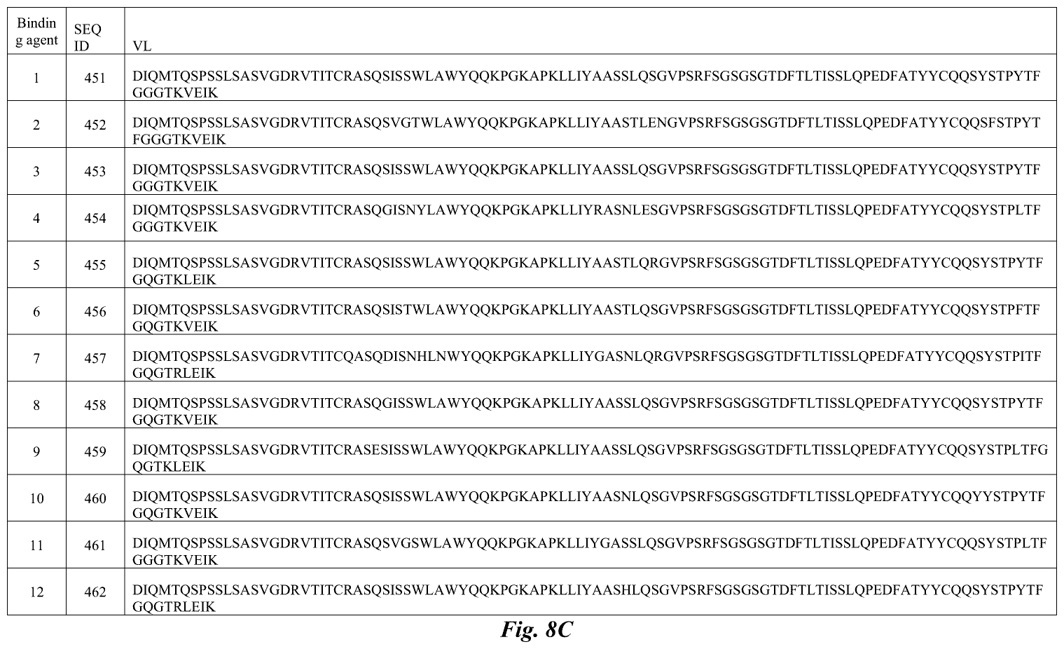

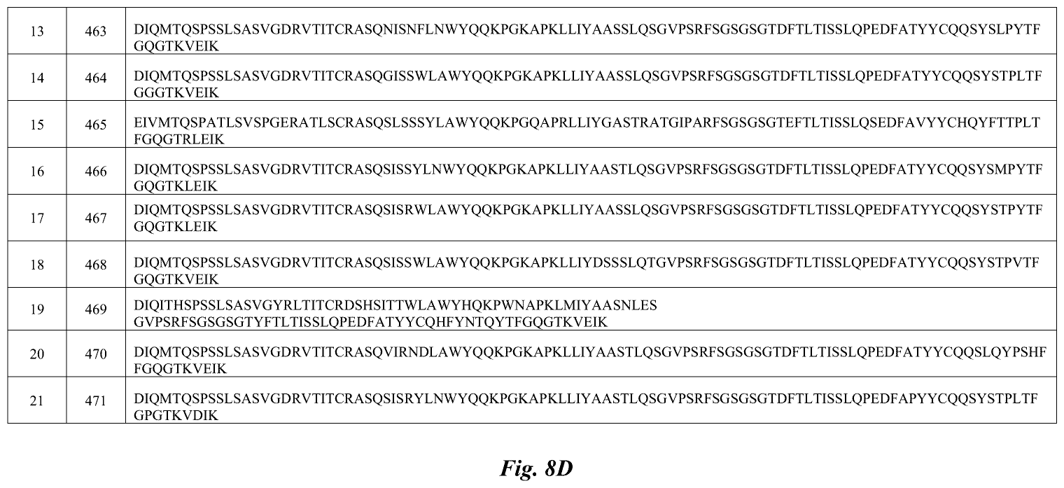

By way of further illustration, the PD-L1 binding agent (Type B) can comprise:
(1) an immunoglobulin heavy chain variable region of SEQ ID NO: 429, or at least the CDRs thereof, and/or an immunoglobulin light chain variable region of SEQ ID NO: 450, or at least the CDRs thereof;
(2) an immunoglobulin heavy chain variable region of SEQ ID NO: 430, or at least the CDRs thereof, and/or an immunoglobulin light chain variable region of SEQ ID NO: 451, or at least the CDRs thereof;
(3) an immunoglobulin heavy chain variable region of SEQ ID NO: 431, or at least the CDRs thereof, and/or an immunoglobulin light chain variable region of SEQ ID NO: 452, or at least the CDRs thereof;
(4) an immunoglobulin heavy chain variable region of SEQ ID NO: 432, or at least the CDRs thereof, and/or an immunoglobulin light chain variable region of SEQ ID NO: 453, or at least the CDRs thereof;
(5) an immunoglobulin heavy chain variable region of SEQ ID NO: 433, or at least the CDRs thereof, and/or an immunoglobulin light chain variable region of SEQ ID NO: 454, or at least the CDRs thereof;
(6) an immunoglobulin heavy chain variable region of SEQ ID NO: 434, or at least the CDRs thereof, and/or an immunoglobulin light chain variable region of SEQ ID NO: 455, or at least the CDRs thereof;
(7) an immunoglobulin heavy chain variable region of SEQ ID NO: 435, or at least the CDRs thereof, and/or an immunoglobulin light chain variable region of SEQ ID NO: 456, or at least the CDRs thereof;
(8) an immunoglobulin heavy chain variable region of SEQ ID NO: 436, or at least the CDRs thereof, and/or an immunoglobulin light chain variable region of SEQ ID NO: 457, or at least the CDRs thereof;
(9) an immunoglobulin heavy chain variable region of SEQ ID NO: 437, or at least the CDRs thereof, and/or an immunoglobulin light chain variable region of SEQ ID NO: 458, or at least the CDRs thereof;
(10) an immunoglobulin heavy chain variable region of SEQ ID NO: 438, or at least the CDRs thereof, and/or an immunoglobulin light chain variable region of SEQ ID NO: 459, or at least the CDRs thereof;
(11) an immunoglobulin heavy chain variable region of SEQ ID NO: 439, or at least the CDRs thereof, and/or an immunoglobulin light chain variable region of SEQ ID NO: 460, or at least the CDRs thereof;
(12) an immunoglobulin heavy chain variable region of SEQ ID NO: 440, or at least the CDRs thereof, and/or an immunoglobulin light chain variable region of SEQ ID NO: 461, or at least the CDRs thereof;
(13) an immunoglobulin heavy chain variable region of SEQ ID NO: 441, or at least the CDRs thereof, and/or an immunoglobulin light chain variable region of SEQ ID NO: 462, or at least the CDRs thereof;
(14) an immunoglobulin heavy chain variable region of SEQ ID NO: 442, or at least the CDRs thereof, and/or an immunoglobulin light chain variable region of SEQ ID NO: 463, or at least the CDRs thereof;
(15) an immunoglobulin heavy chain variable region of SEQ ID NO: 443, or at least the CDRs thereof, and/or an immunoglobulin light chain variable region of SEQ ID NO: 464, or at least the CDRs thereof;
(16) an immunoglobulin heavy chain variable region of SEQ ID NO: 444, or at least the CDRs thereof, and/or an immunoglobulin light chain variable region of SEQ ID NO: 465, or at least the CDRs thereof;
(17) an immunoglobulin heavy chain variable region of SEQ ID NO: 445, or at least the CDRs thereof, and/or an immunoglobulin light chain variable region of SEQ ID NO: 466, or at least the CDRs thereof;
(18) an immunoglobulin heavy chain variable region of SEQ ID NO: 446, or at least the CDRs thereof, and/or an immunoglobulin light chain variable region of SEQ ID NO: 467, or at least the CDRs thereof;
(19) an immunoglobulin heavy chain variable region of SEQ ID NO: 447, or at least the CDRs thereof, and/or an immunoglobulin light chain variable region of SEQ ID NO: 468, or at least the CDRs thereof;
(20) an immunoglobulin heavy chain variable region of SEQ ID NO: 448, or at least the CDRs thereof, and/or an immunoglobulin light chain variable region of SEQ ID NO: 469, or at least the CDRs thereof; and/or
(21) an immunoglobulin heavy chain variable region of SEQ ID NO: 449, or at least the CDRs thereof, and/or an immunoglobulin light chain variable region of SEQ ID NO: 470, or at least the CDRs thereof; and/or
(22) an immunoglobulin heavy chain variable region of FIGS. 8A-B and/or an immunoglobulin light chain variable region of FIGS. 8C-D, or at least the CDRs thereof.

The CDRs of a given heavy or light chain Ig sequence can be determined in accordance with any of the various known Ig numbering schemes (e.g., Kabat, Chothia, Martin (Enhanced Chothia), IGMT, AbM). In certain embodiments, the PD-L1 binding agent comprises one or more of the following CDRs:
a HCDR1 comprising or consisting of any one of SEQ ID NOs: 308-321 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 308-321;
a HCDR2 comprising or consisting of any one of SEQ ID NOs: 322-338 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 322-338; and
a HCDR3 comprising or consisting of any one of SEQ ID NOs: 339-359 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 339-359; and/or the immunoglobulin light chain polypeptide comprises
a LCDR1 comprising or consisting of any one of SEQ ID NOs: 360-374 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 360-374;
a LCDR2 comprising or consisting of any one of SEQ ID NOs: 375-386 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 375-386; and
a LCDR3 comprising or consisting of any one of SEQ ID NOs: 387-398 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 387-398.

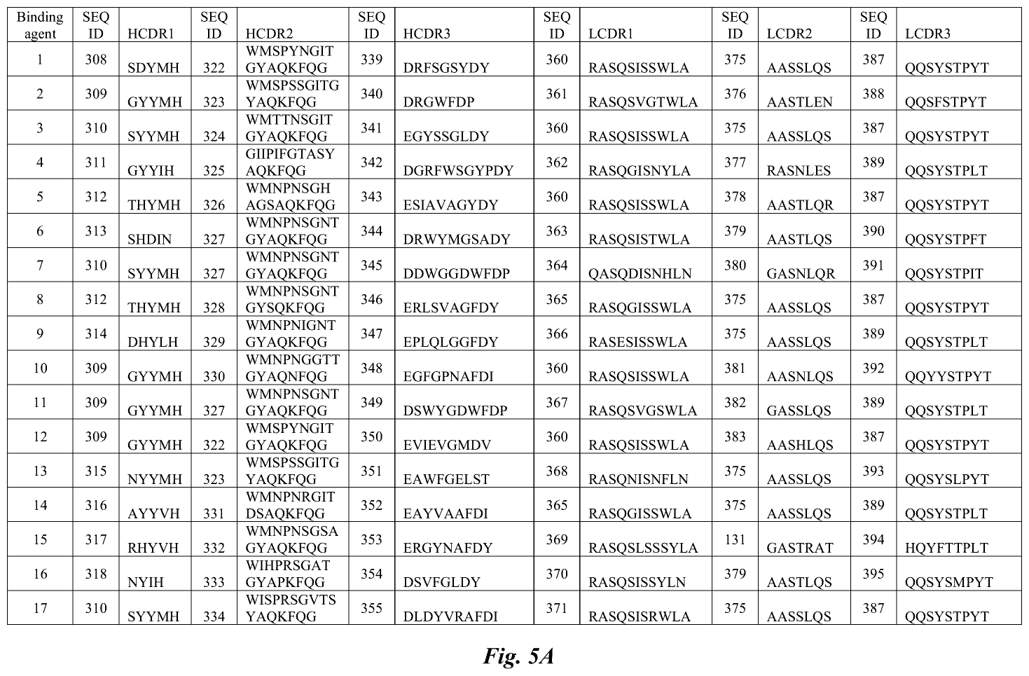

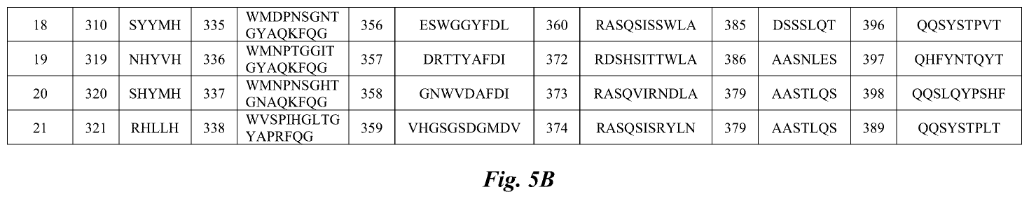

In particular embodiments, the binding agent comprises an immunoglobulin heavy chain polypeptide and an immunoglobulin light chain polypeptide, wherein:
(1) the immunoglobulin heavy chain polypeptide comprises a HCDR1 comprising or consisting of SEQ ID NO: 308, a HCDR2 comprising or consisting of SEQ ID NO: 322, and a HCDR3 comprising or consisting of SEQ ID NO: 339; and/or the immunoglobulin light chain polypeptide comprises a LCDR1 comprising or consisting of SEQ ID NO: 360, a LCDR2 comprising or consisting of SEQ ID NO: 375, and a LCDR3 comprising or consisting of SEQ ID NO: 387;
(2) the immunoglobulin heavy chain polypeptide comprises a HCDR1 comprising or consisting of SEQ ID NO: 309, a HCDR2 comprising or consisting of SEQ ID NO: 323, and a HCDR3 comprising or consisting of SEQ ID NO: 340; and/or the immunoglobulin light chain polypeptide comprises a LCDR1 comprising or consisting of SEQ ID NO: 361, a LCDR2 comprising or consisting of SEQ ID NO: 376, and a LCDR3 comprising or consisting of SEQ ID NO: 388;
(3) the immunoglobulin heavy chain polypeptide comprises a HCDR1 comprising or consisting of SEQ ID NO: 310, a HCDR2 comprising or consisting of SEQ ID NO: 324, and a HCDR3 comprising or consisting of SEQ ID NO: 341; and/or the immunoglobulin light chain polypeptide comprises a LCDR1 comprising or consisting of SEQ ID NO: 360, a LCDR2 comprising or consisting of SEQ ID NO: 375, and a LCDR3 comprising or consisting of SEQ ID NO: 387;
(4) the immunoglobulin heavy chain polypeptide comprises a HCDR1 comprising or consisting of SEQ ID NO: 311, a HCDR2 comprising or consisting of SEQ ID NO: 325, and a HCDR3 comprising or consisting of SEQ ID NO: 342; and/or the immunoglobulin light chain polypeptide comprises a LCDR1 comprising or consisting of SEQ ID NO: 362, a LCDR2 comprising or consisting of SEQ ID NO: 377, and a LCDR3 comprising or consisting of SEQ ID NO: 389;
(5) the immunoglobulin heavy chain polypeptide comprises a HCDR1 comprising or consisting of SEQ ID NO: 312, a HCDR2 comprising or consisting of SEQ ID NO: 326, and a HCDR3 comprising or consisting of SEQ ID NO: 343; and/or the immunoglobulin light chain polypeptide comprises a LCDR1 comprising or consisting of SEQ ID NO: 360, a LCDR2 comprising or consisting of SEQ ID NO: 378, and a LCDR3 comprising or consisting of SEQ ID NO: 387;
(6) the immunoglobulin heavy chain polypeptide comprises a HCDR1 comprising or consisting of SEQ ID NO: 313, a HCDR2 comprising or consisting of SEQ ID NO: 327, and a HCDR3 comprising or consisting of SEQ ID NO: 344; and/or the immunoglobulin light chain polypeptide comprises a LCDR1 comprising or consisting of SEQ ID NO: 363, a LCDR2 comprising or consisting of SEQ ID NO: 379, and a LCDR3 comprising or consisting of SEQ ID NO: 390;
(7) the immunoglobulin heavy chain polypeptide comprises a HCDR1 comprising or consisting of SEQ ID NO: 314, a HCDR2 comprising or consisting of SEQ ID NO: 327, and a HCDR3 comprising or consisting of SEQ ID NO: 345; and/or the immunoglobulin light chain polypeptide comprises a LCDR1 comprising or consisting of SEQ ID NO: 364, a LCDR2 comprising or consisting of SEQ ID NO: 380, and a LCDR3 comprising or consisting of SEQ ID NO: 391;
(8) the immunoglobulin heavy chain polypeptide comprises a HCDR1 comprising or consisting of SEQ ID NO: 312, a HCDR2 comprising or consisting of SEQ ID NO: 328, and a HCDR3 comprising or consisting of SEQ ID NO: 346; and/or the immunoglobulin light chain polypeptide comprises a LCDR1 comprising or consisting of SEQ ID NO: 365, a LCDR2 comprising or consisting of SEQ ID NO: 375, and a LCDR3 comprising or consisting of SEQ ID NO: 387;
(9) the immunoglobulin heavy chain polypeptide comprises a HCDR1 comprising or consisting of SEQ ID NO: 314, a HCDR2 comprising or consisting of SEQ ID NO: 329, and a HCDR3 comprising or consisting of SEQ ID NO: 347; and/or the immunoglobulin light chain polypeptide comprises a LCDR1 comprising or consisting of SEQ ID NO: 366, a LCDR2 comprising or consisting of SEQ ID NO: 375, and a LCDR3 comprising or consisting of SEQ ID NO: 389;
(10) the immunoglobulin heavy chain polypeptide comprises a HCDR1 comprising or consisting of SEQ ID NO: 309, a HCDR2 comprising or consisting of SEQ ID NO: 330, and a HCDR3 comprising or consisting of SEQ ID NO: 348; and/or the immunoglobulin light chain polypeptide comprises a LCDR1 comprising or consisting of SEQ ID NO: 360, a LCDR2 comprising or consisting of SEQ ID NO: 381, and a LCDR3 comprising or consisting of SEQ ID NO: 392;
(11) the immunoglobulin heavy chain polypeptide comprises a HCDR1 comprising or consisting of SEQ ID NO: 309, a HCDR2 comprising or consisting of SEQ ID NO: 327, and a HCDR3 comprising or consisting of SEQ ID NO: 349; and/or the immunoglobulin light chain polypeptide comprises a LCDR1 comprising or consisting of SEQ ID NO: 367, a LCDR2 comprising or consisting of SEQ ID NO: 382, and a LCDR3 comprising or consisting of SEQ ID NO: 389;
(12) the immunoglobulin heavy chain polypeptide comprises a HCDR1 comprising or consisting of SEQ ID NO: 309, a HCDR2 comprising or consisting of SEQ ID NO: 322, and a HCDR3 comprising or consisting of SEQ ID NO: 350; and/or the immunoglobulin light chain polypeptide comprises a LCDR1 comprising or consisting of SEQ ID NO: 360, a LCDR2 comprising or consisting of SEQ ID NO: 383, and a LCDR3 comprising or consisting of SEQ ID NO: 387;
(13) the immunoglobulin heavy chain polypeptide comprises a HCDR1 comprising or consisting of SEQ ID NO: 315, a HCDR2 comprising or consisting of SEQ ID NO: 323, and a HCDR3 comprising or consisting of SEQ ID NO: 351; and/or the immunoglobulin light chain polypeptide comprises a LCDR1 comprising or consisting of SEQ ID NO: 368, a LCDR2 comprising or consisting of SEQ ID NO: 375, and a LCDR3 comprising or consisting of SEQ ID NO: 393;
(14) the immunoglobulin heavy chain polypeptide comprises a HCDR1 comprising or consisting of SEQ ID NO:316, a HCDR2 comprising or consisting of SEQ ID NO: 331, and a HCDR3 comprising or consisting of SEQ ID NO: 352; and/or the immunoglobulin light chain polypeptide comprises a LCDR1 comprising or consisting of SEQ ID NO: 365, a LCDR2 comprising or consisting of SEQ ID NO: 375, and a LCDR3 comprising or consisting of SEQ ID NO: 389;
(15) the immunoglobulin heavy chain polypeptide comprises a HCDR1 comprising or consisting of SEQ ID NO: 317, a HCDR2 comprising or consisting of SEQ ID NO: 332, and a HCDR3 comprising or consisting of SEQ ID NO: 353; and/or the immunoglobulin light chain polypeptide comprises a LCDR1 comprising or consisting of SEQ ID NO: 369, a LCDR2 comprising or consisting of SEQ ID NO: 384, and a LCDR3 comprising or consisting of SEQ ID NO: 394;
(16) the immunoglobulin heavy chain polypeptide comprises a HCDR1 comprising or consisting of SEQ ID NO: 318, a HCDR2 comprising or consisting of SEQ ID NO: 333, and a HCDR3 comprising or consisting of SEQ ID NO: 354; and/or the immunoglobulin light chain polypeptide comprises a LCDR1 comprising or consisting of SEQ ID NO: 370, a LCDR2 comprising or consisting of SEQ ID NO: 379, and a LCDR3 comprising or consisting of SEQ ID NO: 395;
(17) the immunoglobulin heavy chain polypeptide comprises a HCDR1 comprising or consisting of SEQ ID NO:310, a HCDR2 comprising or consisting of SEQ ID NO: 334, and a HCDR3 comprising or consisting of SEQ ID NO: 355; and/or the immunoglobulin light chain polypeptide comprises a LCDR1 comprising or consisting of SEQ ID NO: 371, a LCDR2 comprising or consisting of SEQ ID NO: 375, and a LCDR3 comprising or consisting of SEQ ID NO: 387;
(18) the immunoglobulin heavy chain polypeptide comprises a HCDR1 comprising or consisting of SEQ ID NO:310, a HCDR2 comprising or consisting of SEQ ID NO: 335, and a HCDR3 comprising or consisting of SEQ ID NO: 356; and/or the immunoglobulin light chain polypeptide comprises a LCDR1 comprising or consisting of SEQ ID NO: 360, a LCDR2 comprising or consisting of SEQ ID NO: 385, and a LCDR3 comprising or consisting of SEQ ID NO: 396;
(19) the immunoglobulin heavy chain polypeptide comprises a HCDR1 comprising or consisting of SEQ ID NO: 319, a HCDR2 comprising or consisting of SEQ ID NO: 336, and a HCDR3 comprising or consisting of SEQ ID NO: 357; and/or the immunoglobulin light chain polypeptide comprises a LCDR1 comprising or consisting of SEQ ID NO: 372, a LCDR2 comprising or consisting of SEQ ID NO: 386, and a LCDR3 comprising or consisting of SEQ ID NO: 397;
(20) the immunoglobulin heavy chain polypeptide comprises a HCDR1 comprising or consisting of SEQ ID NO: 320, a HCDR2 comprising or consisting of SEQ ID NO: 337, and a HCDR3 comprising or consisting of SEQ ID NO: 358; and/or the immunoglobulin light chain polypeptide comprises a LCDR1 comprising or consisting of SEQ ID NO: 373, a LCDR2 comprising or consisting of SEQ ID NO: 379, and a LCDR3 comprising or consisting of SEQ ID NO: 398;
(21) the immunoglobulin heavy chain polypeptide comprises a HCDR1 comprising or consisting of SEQ ID NO: 321, a HCDR2 comprising or consisting of SEQ ID NO: 338, and a HCDR3 comprising or consisting of SEQ ID NO: 359; and/or the immunoglobulin light chain polypeptide comprises a LCDR1 comprising or consisting of SEQ ID NO: 374, a LCDR2 comprising or consisting of SEQ ID NO: 379, and a LCDR3 comprising or consisting of SEQ ID NO: 389; and/or
(22) the immunoglobulin heavy chain polypeptide and light chain polypeptide comprises any combination of the CDRs listed in FIGS. 5A-B (Type B).

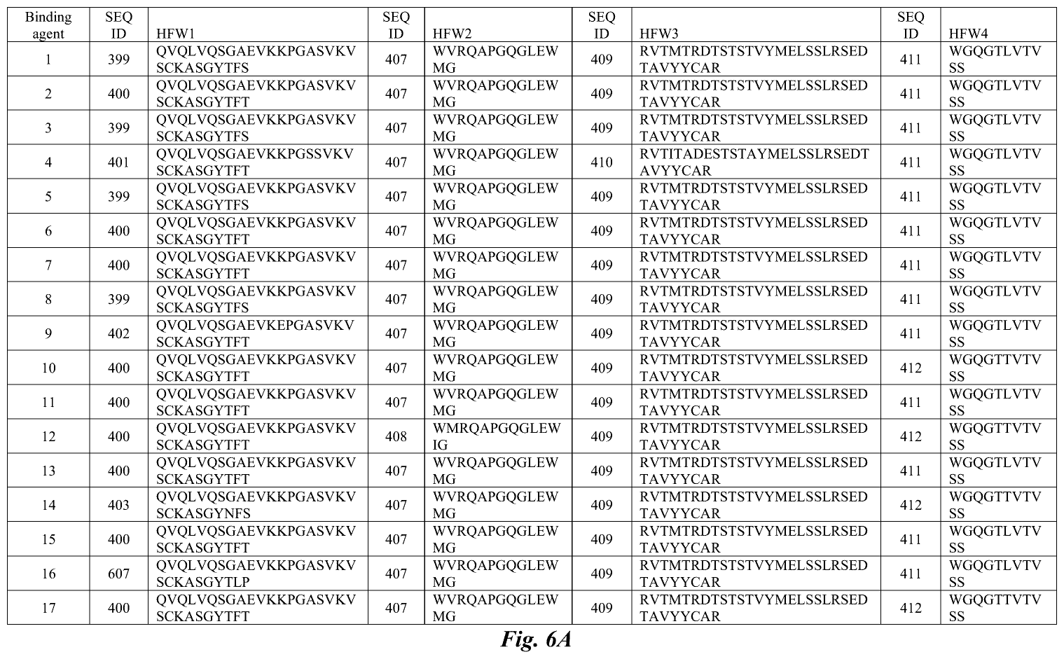

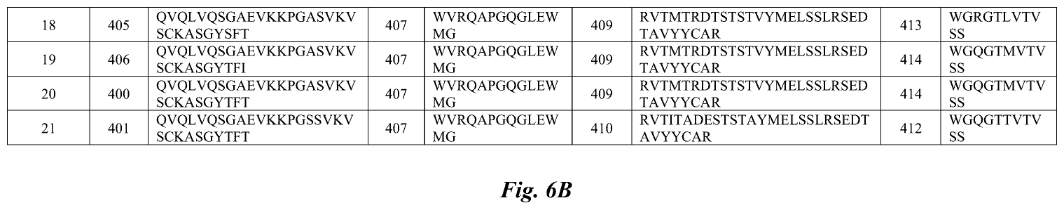

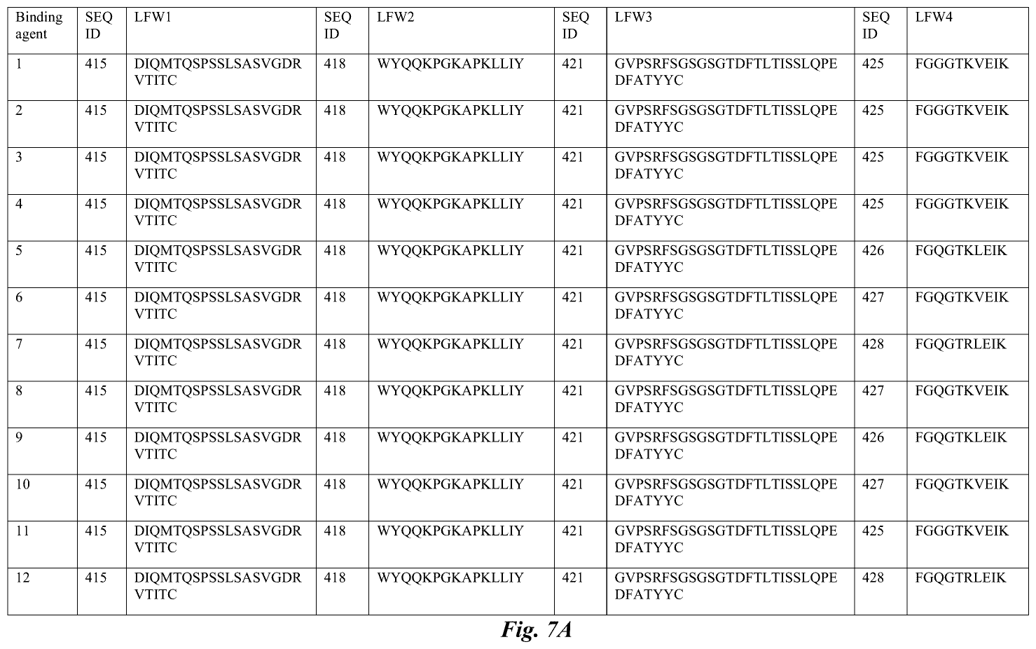

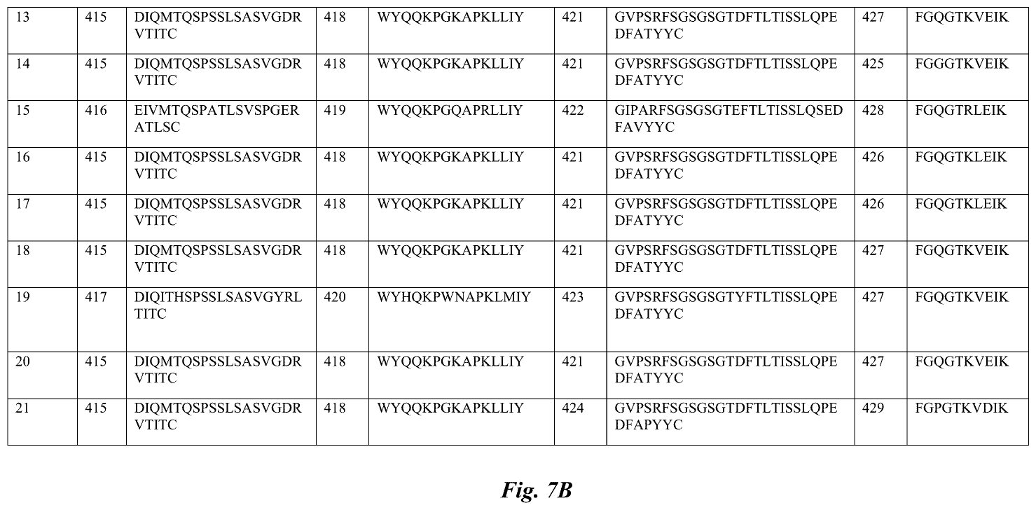

In particular embodiments, the binding agent comprises an immunoglobulin heavy chain polypeptide and an immunoglobulin light chain polypeptide, wherein the immunoglobulin heavy chain polypeptide comprises a first framework region, a second framework region, a third framework region, and/or a fourth framework region; and/or the immunoglobulin light chain polypeptide comprises a first framework region, a second framework region, a third framework region, and/or a fourth framework region; and/or the immunoglobulin heavy chain polypeptide and light chain polypeptide comprises any combination of the framework regions listed in FIGS. 6A-B and/or FIGS. 7A-B (Type B), respectively.

In an exemplary embodiment, the immunoconjugates of the invention comprise an antibody construct that comprises an antigen binding domain that specifically recognizes and binds HER2.

In certain embodiments, immunoconjugates of the invention comprise anti-HER2 antibodies. In one embodiment of the invention, an anti-HER2 antibody of an immunoconjugate of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J. Med. 344:783-792).

In an embodiment of the invention, the antibody construct or antigen binding domain comprises the CDR regions of trastuzumab. In an embodiment of the invention, the anti-HER2 antibody further comprises the framework regions of the trastuzumab. In an embodiment of the invention, the anti-HER2 antibody further comprises one or both variable regions of trastuzumab.

In another embodiment of the invention, an anti-HER2 antibody of an immunoconjugate of the invention comprises a humanized anti-HER2 antibody, e.g., humanized 2C4, as described in U.S. Pat. No. 7,862,817. An exemplary humanized 2C4 antibody is pertuzumab (CAS Reg. No. 380610-27-5), PERJETA™ (Genentech, Inc.). Pertuzumab is a HER dimerization inhibitor (HDI) and functions to inhibit the ability of HER2 to form active heterodimers or homodimers with other HER receptors (such as EGFR/HER1, HER2, HER3 and HER4). See, for example, Harari and Yarden, Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003). PERJETA™ is approved for the treatment of breast cancer.

In an embodiment of the invention, the antibody construct or antigen binding domain comprises the CDR regions of pertuzumab. In an embodiment of the invention, the anti-HER2 antibody further comprises the framework regions of the pertuzumab. In an embodiment of the invention, the anti-HER2 antibody further comprises one or both variable regions of pertuzumab.

In an exemplary embodiment, the immunoconjugates of the invention comprise an antibody construct that comprises an antigen binding domain that specifically recognizes and binds Caprin-1 (Ellis J A, Luzio J P (1995) J Biol Chem. 270(35):20717-23; Wang B, et al (2005) J Immunol. 175 (7):4274-82; Solomon S, et al (2007)Mol Cell Biol. 27(6):2324-42). Caprin-1 is also known as GPIAP1, GPIP137, GRIP137, M11S1, RNG105, p137GPI, and cell cycle associated protein 1.

Cytoplasmic activation/proliferation-associated protein-1 (caprin-1) is an RNA-binding protein that participates in the regulation of cell cycle control-associated genes. Caprin-1 selectively binds to c-Myc and cyclin D2 mRNAs, which accelerates cell progression through the G₁ phase into the S phase, enhances cell viability and promotes cell growth, indicating that it may serve an important role in tumorigenesis (Wang B, et al (2005) J Immunol. 175:4274-4282). Caprin-1 acts alone or in combination with other RNA-binding proteins, such as RasGAP SH3-domain-binding protein 1 and fragile X mental retardation protein. In the tumorigenesis process, caprin-1 primarily functions by activating cell proliferation and upregulating the expression of immune checkpoint proteins. Through the formation of stress granules, caprin-1 is also involved in the process by which tumor cells adapt to adverse conditions, which contributes to radiation and chemotherapy resistance. Given its role in various clinical malignancies, caprin-1 holds the potential to be used as a biomarker and a target for the development of novel therapeutics (Yang, Z-S, et al (2019) Oncology Letters 18:15-21).

Antibodies that target caprin-1 for treatment and detection have been described (WO 2011/096519; WO 2013/125654; WO 2013/125636; WO 2013/125640; WO 2013/125630; WO 2013/018889; WO 2013/018891; WO 2013/018883; WO 2013/018892; WO 2014/014082; WO 2014/014086; WO 2015/020212; WO 2018/079740).

In an exemplary embodiment, the immunoconjugates of the invention comprise an antibody construct that comprises an antigen binding domain that specifically recognizes and binds CEA.

Elevated expression of carcinoembryonic antigen (CEA, CD66e, CEACAM5) has been implicated in various biological aspects of neoplasia, especially tumor cell adhesion, metastasis, the blocking of cellular immune mechanisms, and having antiapoptosis functions. CEA is also used as a blood marker for many carcinomas. Labetuzumab (CEA-CIDE™, Immunomedics, CAS Reg. No. 219649-07-7), also known as MN-14 and hMN14, is a humanized IgG1 monoclonal antibody and has been studied for the treatment of colorectal cancer (Blumenthal, R. et al (2005) Cancer Immunology Immunotherapy 54(4):315-327). Labetuzumab conjugated to a camptothecin analog (labetuzumab govitecan, IMMU-130) targets carcinoembryonic antigen-related cell adhesion mol. 5 (CEACAM5) and is being studied in patients with relapsed or refractory metastatic colorectal cancer (Sharkey, R. et al, (2018), Molecular Cancer Therapeutics 17(1):196-203; Cardillo, T. et al (2018) Molecular Cancer Therapeutics 17(1):150-160).

In an embodiment of the invention, the CEA-targeting antibody construct or antigen binding domain comprises the Variable light chain (VL kappa) of hMN-14/labetuzumab SEQ ID NO. 472 (U.S. Pat. No. 6,676,924).

SEQ ID NO. 472
DIQLTQSPSSLSASVGDRVTITCKASQDVGTSVAWYQQKPGKAPKLLIYW
TSTRHTGVPSRFSGSGSGTDFTFTISSLQPEDIATYYCQQYSLYRSFGQG
TKVEIK

In an embodiment of the invention, the CEA-targeting antibody construct or antigen binding domain comprises the light chain CDR (complementarity determining region) or light chain framework (LFR) sequences of hMN-14/labetuzumab SEQ ID NO. 473-479 (U.S. Pat. No. 6,676,924).

| Region | Sequence Fragment | Residues | Length | SEQ ID NO. |
|---|---|---|---|---|
| LFR1 | DIQLTQSPSSLSASVGDRVTITC | 1-23 | 23 | 473 |
| CDR-L1 | KASQDVGTSVA | 24-34 | 11 | 474 |
| LFR2 | WYQQKPGKAPKLLIY | 35-49 | 15 | 475 |
| CDR-L2 | WTSTRHT | 50-56 | 7 | 476 |
| LFR3 | GVPSRFSGSGSGTDFTFTISSLQPED | 57-88 | 32 | 477 |
| | IATYYC | | | |
| CDR-L3 | QQYSLYRS | 89-96 | 8 | 478 |
| LRF4 | FGQGTKVEIK | 97-106 | 10 | 479 |

In an embodiment of the invention, the CEA-targeting antibody construct or antigen binding domain comprises the Variable heavy chain (VH) of hNM-14/labetuzumab SEQ ID NO. 480 (U.S. Pat. No. 6,676,924).

SEQ ID NO. 480
EVQLVESGGGVVQPGRSLRLSCSSSGFDFTTYWMSWVRQAPGKGLEWVAE
IHPDSSTINYAPSLKDRFTISRDNSKNTLFLQMDSLRPEDTGVYFCASLY
FGFPWFAYWGQGTPVTVSS

In an embodiment of the invention, the CEA-targeting antibody construct or antigen binding domain comprises the heavy chain CDR (complementarity determining region) or heavy chain framework (TIER) sequences of hMVN-14/labetuzumab SEQ ID NO. 481-487 (US 6676924).

| Region | Sequence Fragment | Residues | Length | SEQ ID NO. |
|---|---|---|---|---|
| HFR1 | EVQLVESGGGVVQPGRSLRLSCSSSGFDFT | 1-30 | 30 | 481 |
| CDR-H1 | TYWMS | 31-35 | 5 | 482 |
| HFR2 | WVRQAPGKGLEWVA | 36-49 | 14 | 483 |
| CDR-H2 | EIHPDSSTINYAPSLKD | 50-66 | 17 | 484 |
| HFR3 | RFTISRDNSKNTLFLQMDSLRPEDTGVYFCAS | 67-98 | 32 | 485 |
| CDR-H3 | LYFGFPWFAY | 99-108 | 10 | 486 |
| HFR4 | WGQGTPVTVSS | 109-119 | 11 | 487 |

In an embodiment of the invention, the CEA-targeting antibody construct or antigen binding domain comprises the Variable light chain (VL kappa) of hPR1A3 SEQ ID NO. 488 (U.S. Pat. No. 8,642,742).

SEQ ID NO. 488
DIQMTQSPSSLSASVGDRVTITCKASAAVGTYVAWYQQKPGKAPKLLIYS
ASYRKRGVPSRFSGSGSGTDFTLTISSLQPEDFATYYCHQYYTYPLFTFG
QGTKLEIK

In an embodiment of the invention, the CEA-targeting antibody construct or antigen binding domain comprises the light chain CDR (complementarity determining region) or light chain framework (LFR) sequences of hPR1A3 SEQ ID NO. 489-495 (U.S. Pat. No. 8,642,742).

| Region | Sequence Fragment | Residues | Length | SEQ ID NO. |
|---|---|---|---|---|
| LFR1 | DIQMTQSPSSLSASVGDRVTITC | 1-23 | 23 | 489 |
| CDR-L1 | KASAAVGTYVA | 24-34 | 11 | 490 |
| LFR2 | WYQQKPGKAPKLLIY | 35-49 | 15 | 491 |
| CDR-L2 | SASYRKR | 50-56 | 7 | 492 |
| LFR3 | GVPSRFSGSGSGTDFTLTISSLQPEDFATYYC | 57-88 | 32 | 493 |
| CDR-L3 | HQYYTYPLFT | 89-98 | 10 | 494 |
| LFR4 | FGQGTKLEIK | 99-108 | 10 | 495 |

In an embodiment of the invention, the CEA-targeting antibody construct or antigen binding domain comprises the heavy chain CDR (complementarity determining region) or heavy chain framework (HFR) sequences of hPR1A3 SEQ ID NO. 496-502 (U.S. Pat. No. 8,642,742).

| Region | Sequence Fragment | Residues | Length | SEQ ID NO. |
|---|---|---|---|---|
| HFR1 | QVQLVQSGAEVKKPGASVKVSCKASGYTFT | 1-30 | 30 | 496 |
| CDR-H1 | EFGMN | 31-35 | 5 | 497 |
| HFR2 | WVRQAPGQGLEWMG | 36-49 | 14 | 498 |
| CDR-H2 | WINTKTGEATYVEEFKG | 50-66 | 17 | 499 |
| HFR3 | RVTFTTDTSTSTAYMELRSLRSDDTAVYYCAR | 67-98 | 32 | 500 |
| CDR-H3 | WDFAYYVEAMDY | 99-110 | 12 | 501 |
| HFR4 | WGQGTTVTVSS | 111-121 | 11 | 502 |

In an embodiment of the invention, the CEA-targeting antibody construct or antigen binding domain comprises the Variable light chain (VL kappa) of hMFE-23 SEQ ID NO. 503 (US 723288).

SEQ ID NO. 503
ENVLTQSPSSMSASVGDRVNIACSASSSVSYMHWFQQKPGKSPKLWIYST
SNLASGVPSRFSGSGSGTDYSLTISSMQPEDAATYYCQQRSSYPLTFGGG
TKLEIK

In an embodiment of the invention, the CEA-targeting antibody construct or antigen binding domain comprises the light chain CDR (complementarity determining region) or light chain framework LFR sequences of hMFE-23 SEQ ID NO. 504-510 US 723288

| Region | Sequence Fragment | Residues | Length | SEQ ID NO. |
|---|---|---|---|---|
| LFR1 | ENVLTQSPSSMSASVGDRVNIAC | 1-23 | 23 | 504 |
| CDR-L1 | SASSSVSYMH | 24-33 | 10 | 505 |
| LFR2 | WFQQKPGKSPKLWIY | 34-48 | 15 | 506 |
| CDR-L2 | STSNLAS | 49-55 | 7 | 507 |
| LFR3 | GVPSRFSGSGSGTDYSLTISSMQPEDAATYYC | 56-87 | 32 | 508 |
| CDR-L3 | QQRSSYPLT | 88-96 | 9 | 509 |
| LFR4 | FGGGTKLEIK | 97-106 | 10 | 510 |

In an embodiment of the invention, the CEA-targeting antibody construct or antigen binding domain comprises the Variable heavy chain (VH) of hMFE-23 SEQ ID NO. 511 (US 723288).

SEQ ID NO. 511
QVKLEQSGAEVVKPGASVKLSCKASGFNIKDSYMHWLRQGPGQRLEWIGW
IDPENGDTEYAPKFQGKATFTTDTSANTAYLGLSSLRPEDTAVYYCNEGT
PTGPYYFDYWGQGTLVTVSS

In an embodiment of the invention, the CEA-targeting antibody construct or antigen binding domain comprises the heavy chain CDR (complementarity determining region) or heavy chain framework (TIER) sequences of hMFE-23 SEQ ID NO. 512-518 (US 723288).

| Region | Sequence Fragment | Residues | Length | SEQ ID NO. |
|---|---|---|---|---|
| HFR1 | QVKLEQSGAEVVKPGASVKLSCKASGFNIK | 1-30 | 30 | 512 |
| CDR-H1 | DSYMH | 31-35 | 5 | 513 |
| HFR2 | WLRQGPGQRLEWIG | 36-49 | 14 | 514 |
| CDR-H2 | WIDPENGDTEYAPKFQG | 50-66 | 17 | 515 |
| HFR3 | KATFTTDTSANTAYLGLSSLRPEDTAVYYCNE | 67-98 | 32 | 516 |
| CDR-H3 | GTPTGPYYFDY | 99-109 | 11 | 517 |
| HFR4 | WGQGTLVTVSS | 110-120 | 11 | 518 |

In an embodiment of the invention, the CEA-targeting antibody construct or antigen binding domain comprises the Variable light chain (VL kappa) of SM3E SEQ ID NO. 519 (US 723288).

SEQ ID NO. 519
ENVLTQSPSSMSVSVGDRVTIACSASSSVPYMHWLQQKPGKSPKLLIYLT
SNLASGVPSRFSGSGSGTDYSLTISSVQPEDAATYYCQQRSSYPLTFGGG
TKLEIK

In an embodiment of the invention, the CEA-targeting antibody construct or antigen binding domain comprises the light chain CDR (complementarity determining region) or light chain framework LFR sequences of SM3E SEQ ID NO. 520-526 US 723288).

| Region | Sequence Fragment | Residues | Length | SEQ ID NO. |
|---|---|---|---|---|
| LFR1 | ENVLTQSPSSMSVSVGDRVTIAC | 1-23 | 23 | 520 |
| CDR-L1 | SASSSVPYMH | 24-33 | 10 | 521 |
| LFR2 | WLQQKPGKSPKLLIY | 34-48 | 15 | 522 |
| CDR-L2 | LTSNLAS | 49-55 | 7 | 523 |
| LFR3 | GVPSRFSGSGSGTDYSLTISSVQPEDAATYYC | 56-87 | 32 | 524 |
| CDR-L3 | QQRSSYPLT | 88-96 | 9 | 525 |
| LFR4 | FGGGTKLEIK | 97-106 | 10 | 526 |

In an embodiment of the invention, the CEA-targeting antibody construct or antigen binding domain comprises the Variable heavy chain (VH) of SM3E SEQ ID NO. 527 (US 723288).

SEQ ID NO. 527
QVKLEQSGAEVVKPGASVKLSCKASGFNIKDSYMHWLRQGPGQRLEWIGW
IDPENGDTEYAPKFQGKATFTTDTSANTAYLGLSSLRPEDTAVYYCNEGT
PTGPYYFDYWGQGTLVTVSS

In an embodiment of the invention, the CEA-targeting antibody construct or antigen binding domain comprises the heavy chain CDR (complementarity determining region) or heavy chain framework (TIER) sequences of SM3E SEQ ID NO. 528-534 (US 723288).

| Region | Sequence Fragment | Residues | Length | SEQ ID NO. |
|---|---|---|---|---|
| HFR1 | QVKLEQSGAEVVKPGASVKLSCKASGFNIK | 1-30 | 30 | 528 |
| CDR-H1 | DSYMH | 31-35 | 5 | 529 |
| HFR2 | WLRQGPGQRLEWIG | 36-49 | 14 | 530 |
| CDR-H2 | WIDPENGDTEYAPKFQG | 50-66 | 17 | 531 |
| HFR3 | KATFTTDTSANTAYLGLSSLRPEDTAVYYCNE | 67-98 | 32 | 532 |
| CDR-H3 | GTPTGPYYFDY | 99-109 | 11 | 533 |
| HFR4 | WGQGTLVTVSS | 110-120 | 11 | 534 |

In an embodiment of the invention, the CEA-targeting antibody construct or antigen binding domain comprises the light chain CDR (complementarity determining region) or light chain framework (LFR) sequences of NP-4/arcitumomab SEQ ID NO. 535-541.

| Region | Sequence Fragment | Residues | Length | SEQ ID NO. |
|---|---|---|---|---|
| LFR1 | QTVLSQSPAILSASPGEKVTMTC | 1-23 | 23 | 535 |
| CDR-L1 | RASSSVTYIH | 24-33 | 10 | 536 |
| LFR2 | WYQQKPGSSPKSWIY | 34-48 | 15 | 537 |
| CDR-L2 | ATSNLAS | 49-55 | 7 | 538 |
| LFR3 | GVPARFSGSGSGTSYSLTISRVEAEDAATYYC | 56-87 | 32 | 539 |
| CDR-L3 | QHWSSKPPT | 88-96 | 9 | 540 |
| LFR4 | FGGGTKLEIK | 97-106 | 10 | 541 |

In an embodiment of the invention, the CEA-targeting antibody construct or antigen binding domain comprises the Variable heavy chain (VH) of NP-4/arcitumomab SEQ ID NO. 542.

SEQ ID NO. 542
EVKLVESGGGLVQPGGSLRLSCATSGFTFTDYYMNWVRQPPGKALEWLGF
IGNKANGYTTEYSASVKGRFTISRDKSQSILYLQMNTLRAEDSATYYCTR
DRGLRFYFDYWGQGTTLTVSS.

In an embodiment of the invention, the CEA-targeting antibody construct or antigen binding domain comprises the heavy chain CDR (complementarity determining region) or heavy chain framework H—R sequences of NP-4 SEQ ID NO. 543-549.

| | | | | SEQ |
|---|---|---|---|---|
| | | Resi- | | ID |
| Region | Sequence Fragment | dues | Length | NO. |
| HFR1 | EVKLVESGGGLVQPGGSLR | 1-30 | 30 | 543 |
| | LSCATSGFTFT | | | |
| CDR-H1 | DYYMN | 31-35 | 5 | 544 |
| HFR2 | WVRQPPGKALEWLG | 36-49 | 14 | 545 |
| CDR-H2 | FIGNKANGYTTEYSASVKG | 50-68 | 19 | 546 |
| HFR3 | RFTISRDKSQSILYLQMNT | 69-100 | 32 | 547 |
| | LRAEDSATYYCTR | | | |
| CDR-H3 | DRGLRFYFDY | 101-110 | 10 | 548 |
| HFR4 | WGQGTTLTVSS | 111-121 | 11 | 549 |

In an embodiment of the invention, the CEA-targeting antibody construct or antigen binding domain comprises the Variable light chain (VL kappa) of M5A/hT84.66 SEQ ID NO. 550 (U.S. Pat. No. 7,776,330).

SEQ ID NO. 550
DIQLTQSPSSLSASVGDRVTITCRAGESVDIFGVGFLHWYQQKPGKAPK
LLIYRASNLESGVPSRFSGSGSRTDFTLTISSLQPEDFATYYCQQTNED
PYTFGQGTKVEIK

In an embodiment of the invention, the CEA-targeting antibody construct or antigen binding domain comprises the light chain CDR (complementarity determining region) or light chain framework (LFR) sequences of M5A/hT84.66 SEQ ID NO. 551-557 (U.S. Pat. No. 7,776,330).

| | | | | SEQ |
|---|---|---|---|---|
| | | Resi- | | ID |
| Region | Sequence Fragment | dues | Length | NO. |
| LFR1 | DIQLTQSPSSLSASVGDRV | 1-23 | 23 | 551 |
| | TITC | | | |
| CDR-L1 | RAGESVIDIFGVGFLH | 24-38 | 15 | 552 |
| LFR2 | WYQQKPGKAPKLLIY | 39-53 | 15 | 553 |
| CDR-L2 | RASNLES | 54-60 | 7 | 554 |
| LFR3 | GVPSRFGSGSRTDFTLTIS | 61-92 | 32 | 555 |
| | SLQPEDFATYYC | | | |
| CDR-L3 | QQTNEDPYT | 93-101 | 9 | 556 |
| LFR4 | FGQGTKVEIK | 102-111 | 10 | 557 |

In an embodiment of the invention, the CEA-targeting antibody construct or antigen binding domain comprises the Variable heavy chain (VH) of M5A/hT84.66 SEQ ID NO. 558 (U.S. Pat. No. 7,776,330).

SEQ ID NO. 558
EVQLVESGGGLVQPGGSLRLSCAASGFNIKDTYMHWVRQAPGKGLEWVA
RIDPANGNSKYADSVKGRFTISADTSKNTAYLQMNSLRAEDTAVYYCAP
FGYYVSDYAMAYWGQGTLVTVSS

In an embodiment of the invention, the CEA-targeting antibody construct or antigen binding domain comprises the heavy chain CDR (complementarity determining region) or heavy chain framework (TIER) sequences of M5A/hT84.66 SEQ ID NO. 559-565 (U.S. Pat. No. 7,776,330).

| | | | | SEQ |
|---|---|---|---|---|
| | | Resi- | | ID |
| Region | Sequence Fragment | dues | Length | NO. |
| HFR1 | EVQLVESGGGLVQPGGS | 1-30 | 30 | 559 |
| | LRLSCAASGFNIK | | | |
| CDR-H1 | DTYMH | 31-35 | 5 | 560 |
| HFR2 | WVRPQAPGKGLEWVA | 36-49 | 14 | 561 |
| CDR-H2 | RIDPANGNSKYADSVKG | 50-66 | 17 | 562 |
| HFR3 | RFTISADTSKNTAYLQMN | 67-98 | 32 | 563 |
| | SLRAEDTAVYYCAP | | | |
| CDR-H3 | FGYYVSDYAMAY | 99-110 | 12 | 564 |
| HFR4 | WGQGTLVTVSS | 111-121 | 11 | 565 |

In an embodiment of the invention, the CEA-targeting antibody construct or antigen binding domain comprises the Variable light chain (VL kappa) of ShAb2-3 SEQ ID NO. 566 (U.S. Pat. No. 9,617,345).

SEQ ID NO. 566
DIQMTQSPASLSASVGDRVTITCRASENIFSYLAWYQQKPGKSPKLLVY
NTRTLAEGVPSRFSGSGSGTDFSLTISSLQPEDFATYYCQHHYGTPFTF
GSGTKLEIK

In an embodiment of the invention, the CEA-targeting antibody construct or antigen binding domain comprises the light chain CDR (complementarity determining region) or light chain framework LFR sequences of hAb2-3 SEQ ID NO. 567-573 U.S. Pat. No. 9,617,345).

| | | | | SEQ |
|---|---|---|---|---|
| | | Resi- | | ID |
| Region | Sequence Fragment | dues | Length | NO. |
| LFR1 | DIQMTQSPASLSASVGD | 1-23 | 23 | 567 |
| | RVTITC | | | |
| CDR-L1 | RASENIFSYLA | 24-34 | 11 | 568 |
| LFR2 | WYQQKPGKSPKLLVY | 35-49 | 15 | 569 |
| CDR-L2 | NTRTLAE | 50-56 | 7 | 570 |
| LFR3 | GVPSRFSGSGSGTDFSLT | 57-88 | 37 | 571 |
| | ISSLQPEDFATYYC | | | |
| CDR-L3 | QHHYGTPFT | 89-97 | 9 | 572 |
| LFR4 | FGSGTKLEIK | 98-107 | 10 | 573 |

In an embodiment of the invention, the CEA-targeting antibody construct or antigen binding domain comprises the Variable heavy chain (VH) of SEQ ID NO. 574 (U.S. Pat. No. 9,617,345).

SEQ ID NO. 574
EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSS

In an embodiment of the invention, the CEA-targeting antibody construct or antigen binding domain comprises the heavy chain CDR (complementarity determining region) or heavy chain framework (TIER) sequences of hAb2-3 SEQ ID NO. 575-581.

| | | | | SEQ |
|---|---|---|---|---|
| | | Resi- | | ID |
| Region | Sequence Fragment | dues | Length | NO. |
| HFR1 | EVQLQESGPGLVKPGGSLS | 1-30 | 30 | 575 |
| | LSCAASGFVFS | | | |
| CDR-H1 | SYDMS | 31-35 | 5 | 576 |
| HFR2 | WVRQTPERGLEWVA | 36-49 | 14 | 577 |
| CDR-H2 | YISSGGGITYAPSTVKG | 50-66 | 17 | 578 |
| HFR3 | RFTVSRDNAKNTLYLQMNS | 67-98 | 32 | 579 |
| | LTSEDTAVYYCAA | | | |
| CDR-H3 | HYFGSSGPFAY | 99-109 | 11 | 580 |
| HFR4 | WGQGTLVTVSS | 110-120 | 11 | 581 |

In an embodiment of the invention, the CEA-targeting antibody construct or antigen binding domain comprises the Variable light chain (VL kappa) of A240VL-B9VH/AMG-211 SEQ ID NO. 582 (U.S. Pat. No. 9,982,063).

SEQ ID NO. 582
QAVLTQPASLSASPGASASLTCTLRRGINVGAYSIYWYQQKPGSPPQY
LLRYKSDSDKQQGSGVSSRFSASKDASANAGILLISGLQSEDEADYYC
MIWHSGASAVFGGGTKLTVL

In an embodiment of the invention, the CEA-targeting antibody construct or antigen binding domain comprises the light chain CDR (complementarity determining region) or light chain framework (LFR) sequences of A240VL-B9V7/AMG-2161 SEQ ID NO. 583-589 (US 9982063).

| | | | | SEQ |
|---|---|---|---|---|
| | | Resi- | | ID |
| Region | Sequence Fragment | dues | Length | NO. |
| LFR1 | QAVLTQPASLSASPGASA | 1-22 | 22 | 583 |
| | SLTC | | | |
| CDR-L1 | TLRRGINVGAYSIY | 23-36 | 14 | 584 |
| LFR2 | WYQQKPGSPPQYLLR | 37-51 | 15 | 585 |
| CDR-L2 | YKSDSDKQQGS | 52-62 | 11 | 586 |
| LFR3 | GVSSRFSASKDASANAGIL | 63-96 | 34 | 587 |
| LIS | GLQSEDEADYYC | | | |
| CDR-L3 | MIWHSGASAV | 97-106 | 10 | 588 |
| LFR4 | FGGGTKLTVL | 107-116 | 10 | 589 |

In an embodiment of the invention, the CEA-targeting antibody construct or antigen binding domain comprises the Variable heavy chain (VH) of B9VH SEQ ID NO. 590 (U.S. Pat. No. 9,982,063).

SEQ ID NO. 590
EVQLVESGGGLVQPGRSLRLSCAASGFTVSSYWMHWVRQAPGKGLEW
VGFIRNKANGGTTEYAASVKGRFTISRDDSKNTLYLQMNSLRAEDTA
VYYCARDRGLRFYFDYWGQGTTVTVSS

In an embodiment of the invention, the CEA-targeting antibody construct or antigen binding domain comprises the heavy chain CDR (complementarity determining region) or heavy chain framework (TIER) sequences of SEQ ID NO. 591-598 (U.S. Pat. No. 9,982,063). The embodiment includes two variants of CDR-H2, SEQ ID NO.:594 and SEQ ID NO.:595.

| | | | | SEQ |
|---|---|---|---|---|
| | | Resi- | | ID |
| Region | Sequence Fragment | dues | Length | NO. |
| HFR1 | EVQLVESGGGLVQPGRSL | 1-30 | 30 | 591 |
| | RLSCAASGFTVS | | | |
| CDR-H1 | SYWMH | 31-35 | 5 | 592 |
| HFR2 | WVRQAPGKGLEWVG | 36-49 | 14 | 593 |
| CDR-H2 | FIRNKANGGTTEYAASVKG | 50-68 | 19 | 594 |
| CDR-H2 | FIRNKANSGTTEYAASVKG | 50-68 | 19 | 595 |
| HFR3 | RFTISRDDSKNTLYLQMN | 69-100 | 32 | 596 |
| | SLRAEDTAVYYCAR | | | |
| CDR-H3 | DRGLRFYFDY | 101-110 | 10 | 597 |
| HFR4 | WGQGTTVTVSS | 111-121 | 11 | 598 |

In an embodiment of the invention, the CEA-targeting antibody construct or antigen binding domain comprises the Variable heavy chain (VH) of E12VH SEQ ID NO. 599 (U.S. Pat. No. 9,982,063).

SEQ ID NO. 599
EVQLVESGGGLVQPGRSLRLSCAASGFTVSSYWMHWVRQAPGKGLE
WVGFILNKANGGTTEYAASVKGRFTISRDDSKNTLYLQMNSLRAED
TAVYYCARDRGLRFYFDYWGQGTTVTVSS

In an embodiment of the invention, the CEA-targeting antibody construct or antigen binding domain comprises the heavy chain CDR (complementarity determining region) or heavy chain framework (HFR) sequences of SEQ ID NO. 600-606 (U.S. Pat. No. 9,982,063).

| | | | | SEQ |
|---|---|---|---|---|
| | | Resi- | | ID |
| Region | Sequence Fragment | dues | Length | NO. |
| HFR1 | EVQLVESGGGLVQPGRS | 1-30 | 30 | 600 |
| | LRLSCAASGFTVS | | | |
| CDR-H1 | SYWMH | 31-35 | 5 | 601 |
| HFR2 | WVRQAPGKGLEWVG | 36-49 | 14 | 602 |
| CDR-H2 | FILNKANGGTTEYAASVKG | 50-68 | 19 | 603 |
| HFR3 | RFTISRDDSKNTLYLQM | 69-100 | 32 | 604 |
| | NSLRAEDTAVYYCAR | | | |
| CDR-H3 | DRGLRFYFDY | 101-110 | 10 | 605 |
| HFR4 | WGQGTTVTVSS | 111-121 | 11 | 606 |

In some embodiments, the antibody construct further comprises an Fc domain. In certain embodiments, the antibody construct is an antibody. In certain embodiments, the antibody construct is a fusion protein. The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. The antibody construct or antigen binding domain may comprise one or more variable regions (e.g., two variable regions) of an antigen binding domain of an anti-PD-L1 antibody, an anti-HER2 antibody, or an anti-CEA antibody, each variable region comprising a CDR1, a CDR2, and a CDR3.

In some embodiments, the antibodies in the immunoconjugates contain a modified Fc region, wherein the modification modulates the binding of the Fc region to one or more Fc receptors.

In some embodiments, the Fc region is modified by inclusion of a transforming growth factor beta 1 (TGFβ1) receptor, or a fragment thereof, that is capable of binding TGFβ1. For example, the receptor can be TGFβ receptor II (TGFβRII). In some embodiments, the TGFβ receptor is a human TGFβ receptor. In some embodiments, the IgG has a C-terminal fusion to a TGFβRII extracellular domain (ECD) as described in U.S. Pat. No. 9,676,863, incorporated herein. An “Fc linker” may be used to attach the IgG to the TGFβRII extracellular domain, for example, a G₄S4G Fc linker (SEQ ID NO: 608). The Fc linker may be a short, flexible peptide that allows for the proper three-dimensional folding of the molecule while maintaining the binding-specificity to the targets. In some embodiments, the N-terminus of the TGFβ receptor is fused to the Fc of the antibody construct (with or without an Fc linker). In some embodiments, the C-terminus of the antibody construct heavy chain is fused to the TGFβ receptor (with or without an Fc linker). In some embodiments, the C-terminal lysine residue of the antibody construct heavy chain is mutated to alanine.

In some embodiments, the antibodies in the immunoconjugates are glycosylated.

In some embodiments, the antibodies in the immunoconjugates is a cysteine-engineered antibody which provides for site-specific conjugation of an adjuvant, label, or drug moiety to the antibody through cysteine substitutions at sites where the engineered cysteines are available for conjugation but do not perturb immunoglobulin folding and assembly or alter antigen binding and effector functions (Junutula, et al., 2008b Nature Biotech., 26(8):925-932; Dornan et al. (2009) Blood 114(13):2721-2729; U.S. Pat. Nos. 7,521,541; 7,723,485; US 2012/0121615; WO 2009/052249). A “cysteine engineered antibody” or “cysteine engineered antibody variant” is an antibody in which one or more residues of an antibody are substituted with cysteine residues. Cysteine-engineered antibodies can be conjugated to the aminobenzazepine adjuvant moiety as an aminobenzazepine-linker compound with uniform stoichiometry (e.g., up to 2 aminobenzazepine moieties per antibody in an antibody that has a single engineered cysteine site).

In some embodiments, cysteine-engineered antibodies used to prepare the immunoconjugates of Table 3 have a cysteine residue introduced at the 149-lysine site of the light chain (LC K149C). In other embodiments, the cysteine-engineered antibodies have a cysteine residue introduced at the 118-alanine site (EU numbering) of the heavy chain (HC A118C). This site is alternatively numbered 121 by Sequential numbering or 114 by Kabat numbering. In other embodiments, the cysteine-engineered antibodies have a cysteine residue introduced in the light chain at G64C or R142C according to Kabat numbering, or in the heavy chain at D101C, V184C or T205C according to Kabat numbering.

### Aminobenzazepine Adjuvant Compounds

The immunoconjugate of the invention comprises an aminobenzazepine adjuvant moiety. The adjuvant moiety described herein is a compound that elicits an immune response (i.e., an immunostimulatory agent). Generally, the adjuvant moiety described herein is a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-KB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-βB inhibitor I-βB. As a result, NF-κB enters the cell nucleus and initiates transcription of genes whose promoters contain NF-βB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Typically, the adjuvant moiety described herein is a TLR7 and/or TLR8 agonist. TLR7 and TLR8 are both expressed in cells of myeloid lineage (e.g. monocytes and dendritic cells). In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

Exemplary aminobenzazepine compounds (Bz) of the invention are shown in Tables 1a, 1b, and 1c. Each compound was synthesized and purified by the methods in the Examples provided herein, characterized by mass spectrometry, and shown to have the mass indicated. Activity against HEK293 NFKB reporter cells expressing human TLR7 or human TLR8 was measured according to Example 68. The aminobenzazepine compounds of Tables 1a, 1b, and 1c demonstrate the surprising and unexpected property of TLR8 agonist selectivity which may predict useful therapeutic activity to treat cancer and other disorders.

TABLE 1a
Aminobenzazepine compounds (Bz)

| | | | HEK293 | HEK293 |
|---|---|---|---|---|
| Bz | | | hTLR7 | hTLR8 |
| No. | Structure | MW | EC50 (nM) | EC50 (nM) |
| Bz-1 | 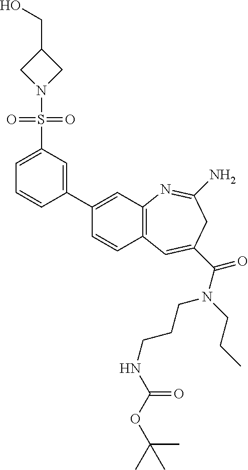 [SMILES: CCCN(CCCNC(=O)OC(C)(C)C)C(=O)C1=Cc2ccc(-c3cccc(S(=O)(=O)N4CC(CO)C4)c3)cc2N=C(N)C1] | 625.8 | 571 | 106 |
| Bz-2 | 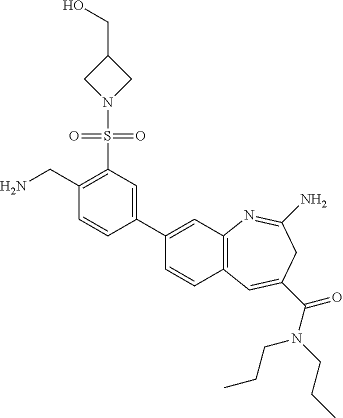 [SMILES: CCCN(CCC)C(=O)C1=Cc2ccc(-c3ccc(CN)c(S(=O)(=O)N4CC(CO)C4)c3)cc2N=C(N)C1] | 538.7 | >9000 | 9760 |
| Bz-3 | 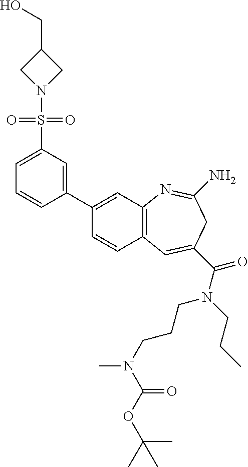 [SMILES: CCCN(CCCN(C)C(=O)OC(C)(C)C)C(=O)C1=Cc2ccc(-c3cccc(S(=O)(=O)N4CC(CO)C4)c3)cc2N=C(N)C1] | 639.8 | 545.2 | 4306 |
| Bz-4 | 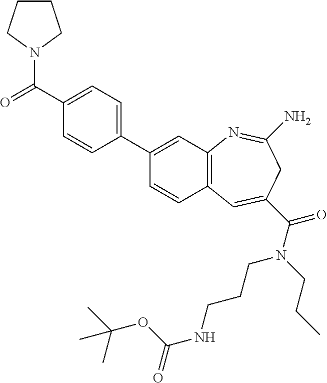 [SMILES: CCCN(CCCNC(=O)OC(C)(C)C)C(=O)C1=Cc2ccc(-c3ccc(C(=O)N4CCCC4)cc3)cc2N=C(N)C1] | 573.7 | 1484 | 1681 |
| Bz-5 | 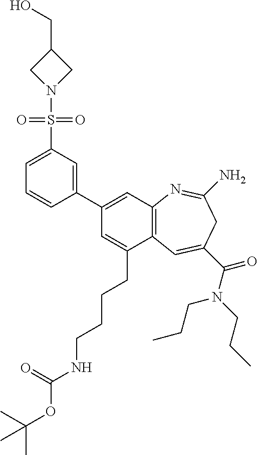 [SMILES: CCCN(CCC)C(=O)C1=Cc2c(CCCCNC(=O)OC(C)(C)C)cc(-c3cccc(S(=O)(=O)N4CC(CO)C4)c3)cc2N=C(N)C1] | 681.9 | 155.2 | 255.5 |
| Bz-6 | 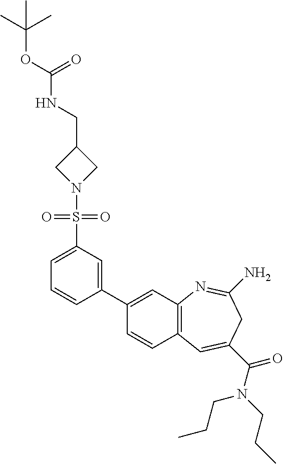 [SMILES: CCCN(CCC)C(=O)C1=Cc2ccc(-c3cccc(S(=O)(=O)N4CC(CNC(=O)OC(C)(C)C)C4)c3)cc2N=C(N)C1] | 609.8 | >9000 | 264.7 |
| Bz-7 | 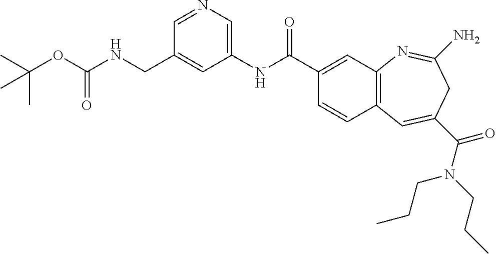 [SMILES: CCCN(CCC)C(=O)C1=Cc2ccc(C(=O)Nc3cncc(CNC(=O)OC(C)(C)C)c3)cc2N=C(N)C1] | 534.7 | >9000 | 4.283 |
| Bz-8 | 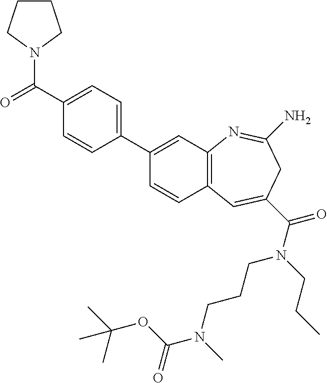 [SMILES: CCCN(CCCN(C)C(=O)OC(C)(C)C)C(=O)C1=Cc2ccc(-c3ccc(C(=O)N4CCCC4)cc3)cc2N=C(N)C1] | 587.8 | 3367 | >9000 |
| Bz-9 | 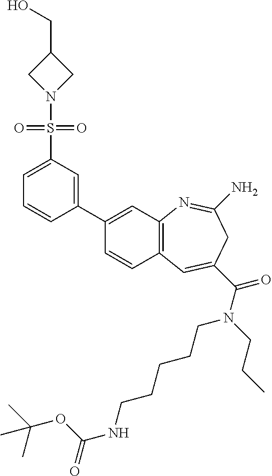 [SMILES: CCCN(CCCCCNC(=O)OC(C)(C)C)C(=O)C1=Cc2ccc(-c3cccc(S(=O)(=O)N4CC(CO)C4)c3)cc2N=C(N)C1] | 653.8 | 8647 | 629.1 |
| Bz-10 | 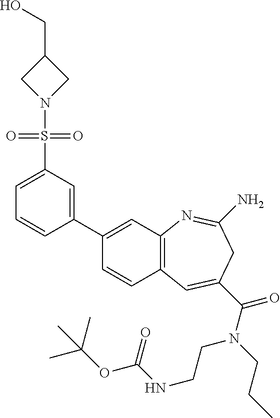 [SMILES: CCCN(CCNC(=O)OC(C)(C)C)C(=O)C1=Cc2ccc(-c3cccc(S(=O)(=O)N4CC(CO)C4)c3)cc2N=C(N)C1] | 611.8 | >9000 | >9000 |
| Bz-11 | 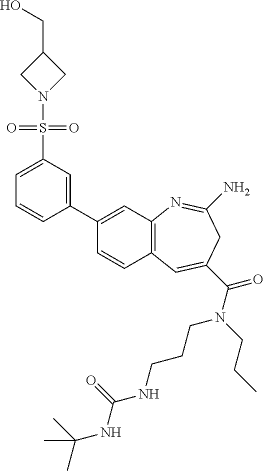 [SMILES: CCCN(CCCNC(=O)NC(C)(C)C)C(=O)C1=Cc2ccc(-c3cccc(S(=O)(=O)N4CC(CO)C4)c3)cc2N=C(N)C1] | 624.8 | 7843 | 1387 |
| Bz-12 | 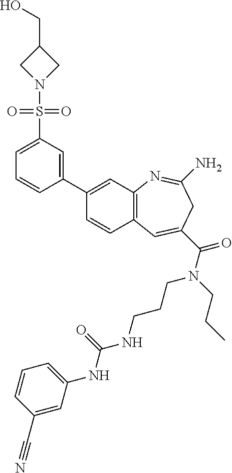 [SMILES: CCCN(CCCNC(=O)Nc1cccc(C#N)c1)C(=O)C1=Cc2ccc(-c3cccc(S(=O)(=O)N4CC(CO)C4)c3)cc2N=C(N)C1] | 669.8 | 2487 | 2375 |
| Bz-13 | 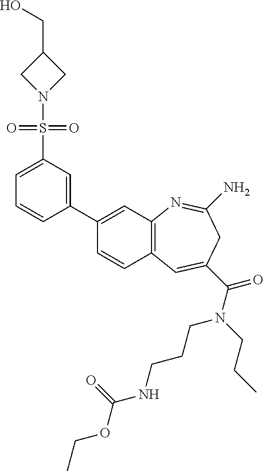 [SMILES: CCCN(CCCNC(=O)OCC)C(=O)C1=Cc2ccc(-c3cccc(S(=O)(=O)N4CC(CO)C4)c3)cc2N=C(N)C1] | 597.7 | 1371 | 134 |
| Bz-14 | 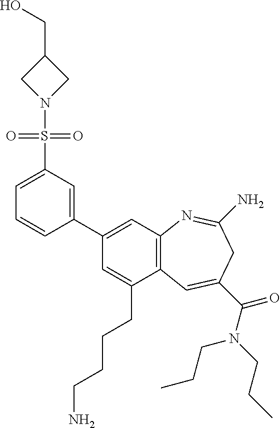 [SMILES: CCCN(CCC)C(=O)C1=Cc2c(CCCCN)cc(-c3cccc(S(=O)(=O)N4CC(CO)C4)c3)cc2N=C(N)C1] | 581.8 | >9000 | 1700 |
| Bz-15 | 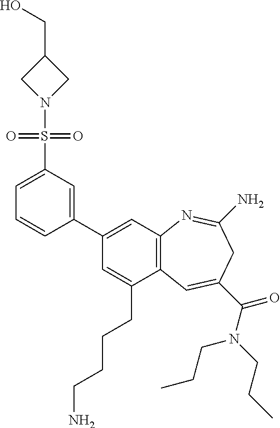 [SMILES: CCCN(CCC)C(=O)C1=Cc2c(CCCCN)cc(-c3cccc(S(=O)(=O)N4CC(CO)C4)c3)cc2N=C(N)C1] | 509.7 | >9000 | 103 |
| Bz-16 | 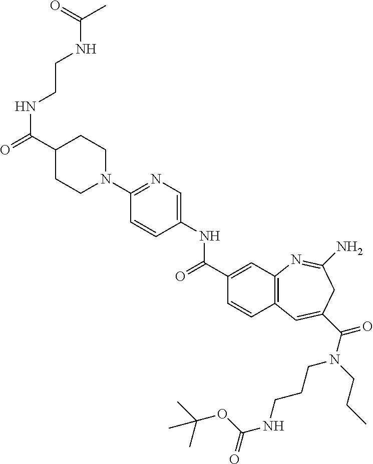 [SMILES: CCCN(CCCNC(=O)OC(C)(C)C)C(=O)C1=Cc2ccc(C(=O)Nc3ccc(N4CCC(C(=O)NCCNC(C)=O)CC4)nc3)cc2N=C(N)C1] | 731.9 | >9000 | 1047 |

TABLE 1b
Aminobenzazepine compounds (Bz)

| | | | HEK293 | HEK293 |
|---|---|---|---|---|
| Bz | | | hTLR7 | hTLR8 |
| No. | Structure | MW | EC50 (nM) | EC50 (nM) |
| Bz-17 | 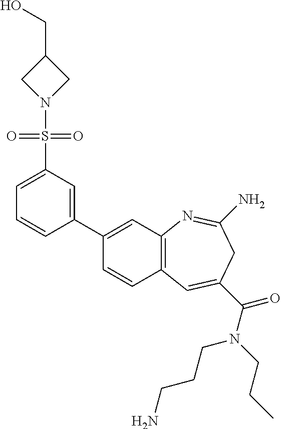 [SMILES: CCCN(CCCN)C(=O)C1=Cc2ccc(-c3cccc(S(=O)(=O)N4CC(CO)C4)c3)cc2N=C(N)C1] | 525.7 | >9000 | >9000 |
| Bz-18 | 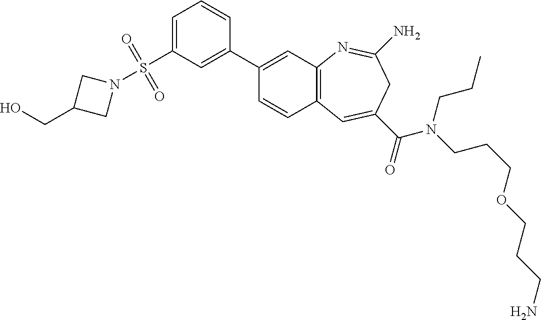 [SMILES: CCCN(CCCOCCCN)C(=O)C1=Cc2ccc(-c3cccc(S(=O)(=O)N4CC(CO)C4)c3)cc2N=C(N)C1] | 583.7 | 1994 | 3403 |
| Bz-19 | 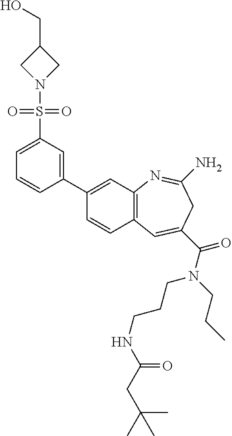 [SMILES: CCCN(CCCNC(=O)CC(C)(C)C)C(=O)C1=Cc2ccc(-c3cccc(S(=O)(=O)N4CC(CO)C4)c3)cc2N=C(N)C1] | 623.8 | 1067 | 3168 |
| Bz-20 | 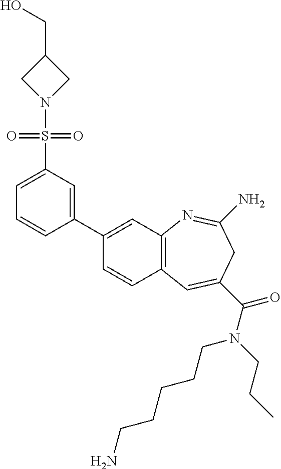 [SMILES: CCCN(CCCCCN)C(=O)C1=Cc2ccc(-c3cccc(S(=O)(=O)N4CC(CO)C4)c3)cc2N=C(N)C1] | 553.7 | >9000 | >9000 |
| Bz-21 | 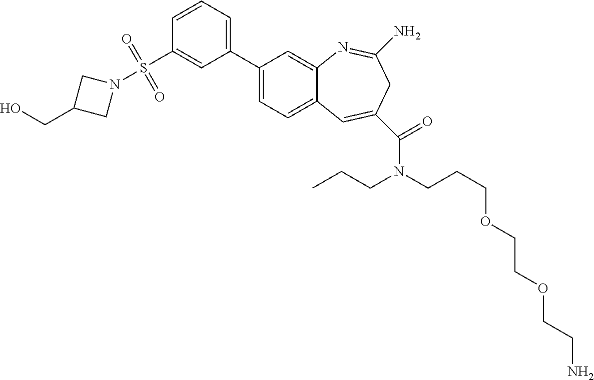 [SMILES: CCCN(CCCOCCOCCN)C(=O)C1=Cc2ccc(-c3cccc(S(=O)(=O)N4CC(CO)C4)c3)cc2N=C(N)C1] | 613.8 | >9000 | >9000 |
| Bz-22 | 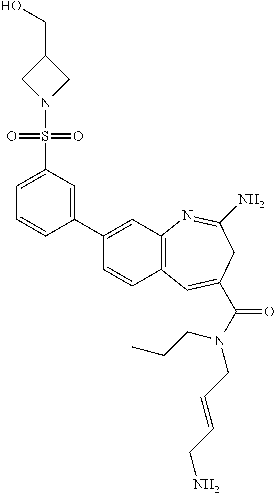 [SMILES: CCCN(C/C=C/CN)C(=O)C1=Cc2ccc(-c3cccc(S(=O)(=O)N4CC(CO)C4)c3)cc2N=C(N)C1] | 537.7 | >9000 | >9000 |
| Bz-23 | 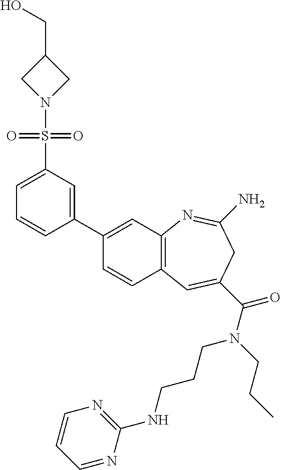 [SMILES: CCCN(CCCNc1ncccn1)C(=O)C1=Cc2ccc(-c3cccc(S(=O)(=O)N4CC(CO)C4)c3)cc2N=C(N)C1] | 603.7 | 2427 | 1162 |
| Bz-24 | 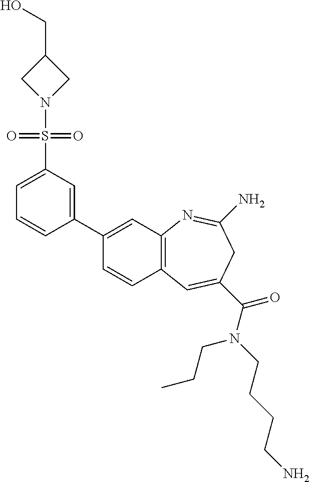 [SMILES: CCCN(CCCCN)C(=O)C1=Cc2ccc(-c3cccc(S(=O)(=O)N4CC(CO)C4)c3)cc2N=C(N)C1] | 539.7 | >9000 | >9000 |
| Bz-25 | 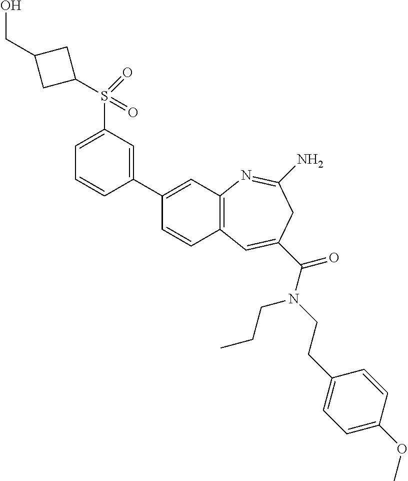 [SMILES: CCCN(CCc1ccc(OC)cc1)C(=O)C1=Cc2ccc(-c3cccc(S(=O)(=O)C4CC(CO)C4)c3)cc2N=C(N)C1] | 602.8 | >9000 | 1403 |
| Bz-26 | 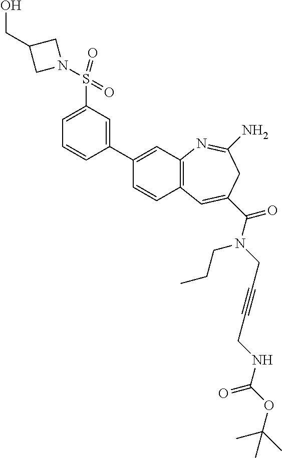 [SMILES: CCCN(CC#CCNC(=O)OC(C)(C)C)C(=O)C1=Cc2ccc(-c3cccc(S(=O)(=O)N4CC(CO)C4)c3)cc2N=C(N)C1] | 635.8 | >9000 | 318 |
| Bz-27 | 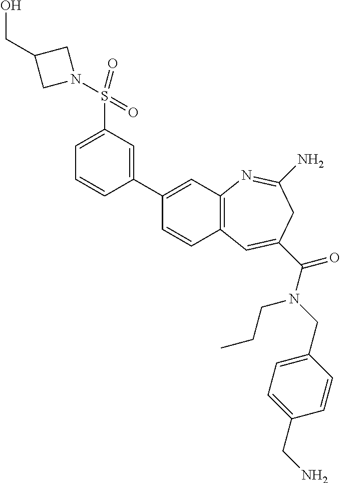 [SMILES: CCCN(Cc1ccc(CN)cc1)C(=O)C1=Cc2ccc(-c3cccc(S(=O)(=O)N4CC(CO)C4)c3)cc2N=C(N)C1] | 587.7 | >9000 | 138 |
| Bz-28 | 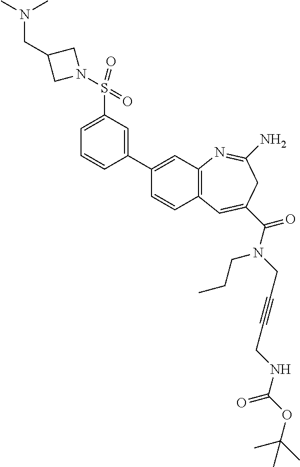 [SMILES: CCCN(CC#CCNC(=O)OC(C)(C)C)C(=O)C1=Cc2ccc(-c3cccc(S(=O)(=O)N4CC(CN(C)C)C4)c3)cc2N=C(N)C1] | 662.9 | 4253.9 | 42.8 |
| Bz-29 | 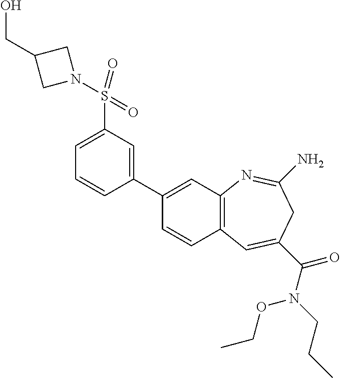 [SMILES: CCCN(OCC)C(=O)C1=Cc2ccc(-c3cccc(S(=O)(=O)N4CC(CO)C4)c3)cc2N=C(N)C1] | 512.6 | >9000 | 32 |
| Bz-30 | 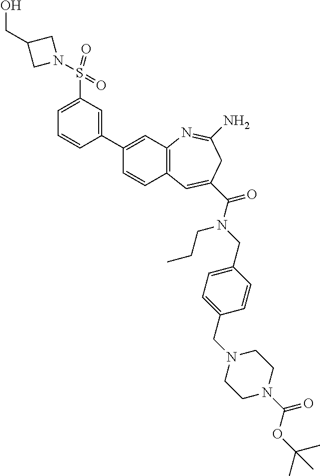 [SMILES: CCCN(Cc1ccc(CN2CCN(C(=O)OC(C)(C)C)CC2)cc1)C(=O)C1=Cc2ccc(-c3cccc(S(=O)(=O)N4CC(CO)C4)c3)cc2N=C(N)C1] | 757.0 | >9000 | 1022.3 |
| Bz-31 | 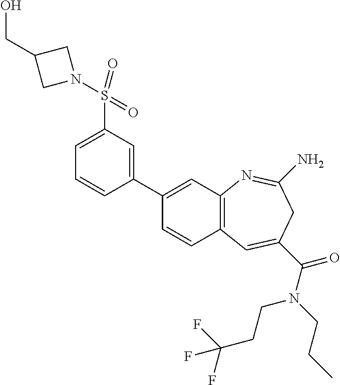 [SMILES: CCCN(CCC(F)(F)F)C(=O)C1=Cc2ccc(-c3cccc(S(=O)(=O)N4CC(CO)C4)c3)cc2N=C(N)C1] | 564.6 | >9000 | 341 |
| Bz-32 | 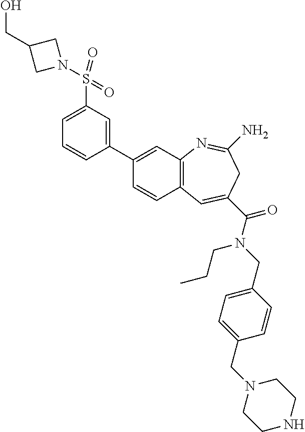 [SMILES: CCCN(Cc1ccc(CN2CCNCC2)cc1)C(=O)C1=Cc2ccc(-c3cccc(S(=O)(=O)N4CC(CO)C4)c3)cc2N=C(N)C1] | 656.8 | >9000 | >9000 |
| Bz-33 | 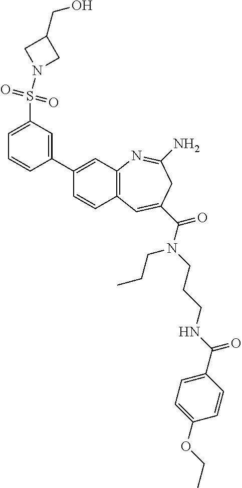 [SMILES: CCCN(CCCNC(=O)c1ccc(OCC)cc1)C(=O)C1=Cc2ccc(-c3cccc(S(=O)(=O)N4CC(CO)C4)c3)cc2N=C(N)C1] | 673.8 | 1428 | 1919 |
| Bz-34 | 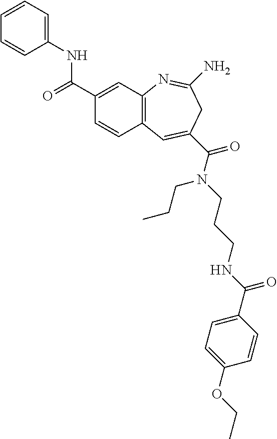 [SMILES: CCCN(CCCNC(=O)c1ccc(OCC)cc1)C(=O)C1=Cc2ccc(C(=O)Nc3ccccc3)cc2N=C(N)C1] | 567.7 | >9000 | 1040 |

TABLE 1c
Aminobenzazepine compounds (Bz)

| | | | HEK293 | HEK293 |
|---|---|---|---|---|
| | | | hTLR7 | hTLR8 |
| Bz | | | EC50 | EC50 |
| No. | Structure | MW | (nM) | (nM) |
| Bz-35 | 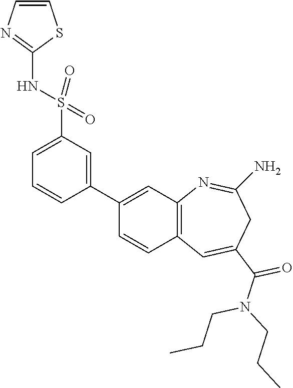 [SMILES: CCCN(CCC)C(=O)C1=Cc2ccc(-c3cccc(S(=O)(=O)Nc4nccs4)c3)cc2N=C(N)C1] | 523.7 | 9000 | 9000 |
| Bz-36 | 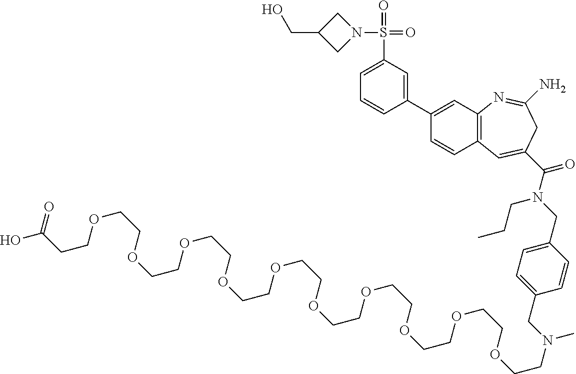 [SMILES: CCCN(Cc1ccc(CN(C)CCOCCOCCOCCOCCOCCOCCOCCOCCOCCOCCC(=O)O)cc1)C(=O)C1=Cc2ccc(-c3cccc(S(=O)(=O)N4CC(CO)C4)c3)cc2N=C(N)C1] | 1114.4 | ND | ND |
| Bz-37 | 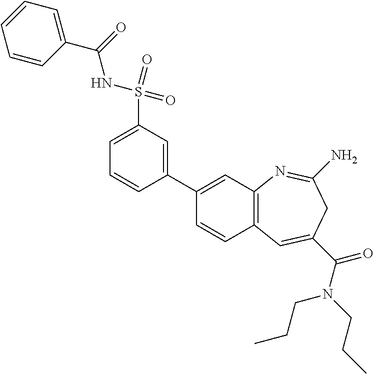 [SMILES: CCCN(CCC)C(=O)C1=Cc2ccc(-c3cccc(S(=O)(=O)NC(=O)c4ccccc4)c3)cc2N=C(N)C1] | 544.7 | 9000 | 9000 |
| Bz-38 | 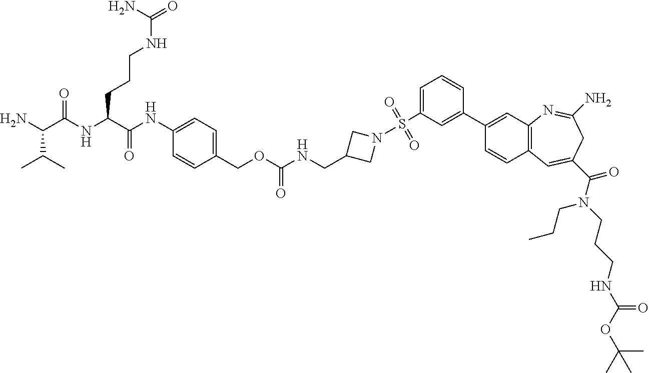 [SMILES: CCCN(CCCNC(=O)OC(C)(C)C)C(=O)C1=Cc2ccc(-c3cccc(S(=O)(=O)N4CC(CNC(=O)OCc5ccc(NC(=O)[C@H](CCCNC(N)=O)NC(=O)[C@@H](N)C(C)C)cc5)C4)c3)cc2N=C(N)C1] | 1030.2 | ND | ND |
| Bz-39 | 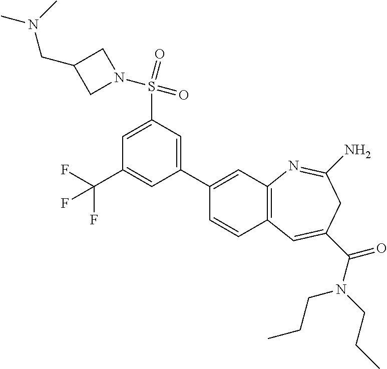 [SMILES: CCCN(CCC)C(=O)C1=Cc2ccc(-c3cc(C(F)(F)F)cc(S(=O)(=O)N4CC(CN(C)C)C4)c3)cc2N=C(N)C1] | 605.7 | 42 | 728 |
| Bz-40 | 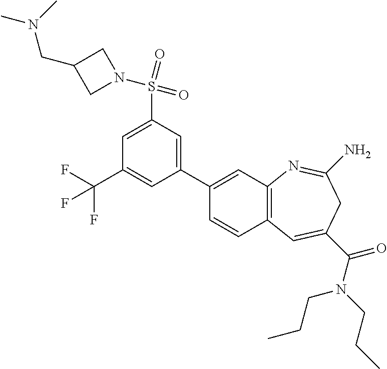 [SMILES: CCCN(CCC)C(=O)C1=Cc2ccc(-c3cc(C(F)(F)F)cc(S(=O)(=O)N4CC(CN(C)C)C4)c3)cc2N=C(N)C1] | 509.8 | 332 | 9000 |
| Bz-41 | 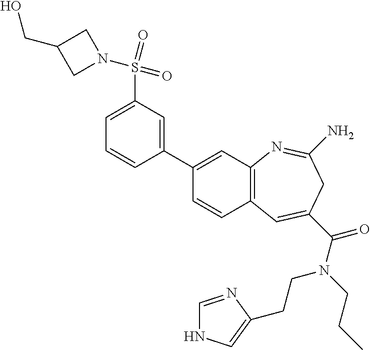 [SMILES: CCCN(CCc1c[nH]cn1)C(=O)C1=Cc2ccc(-c3cccc(S(=O)(=O)N4CC(CO)C4)c3)cc2N=C(N)C1] | 562.7 | 9000 | 9000 |
| Bz-42 | 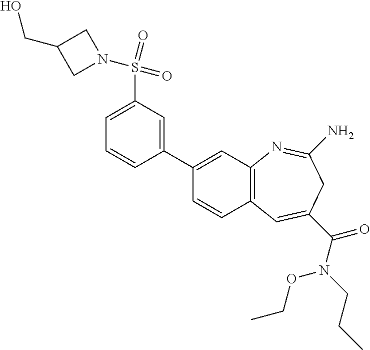 [SMILES: CCCN(OCC)C(=O)C1=Cc2ccc(-c3cccc(S(=O)(=O)N4CC(CO)C4)c3)cc2N=C(N)C1] | 512.6 | 9000 | 49 |
| Bz-43 | 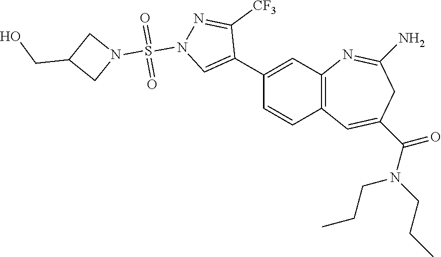 [SMILES: CCCN(CCC)C(=O)C1=Cc2ccc(-c3cn(S(=O)(=O)N4CC(CO)C4)nc3C(F)(F)F)cc2N=C(N)C1] | 568.6 | 9000 | 7005 |
| Bz-44 | 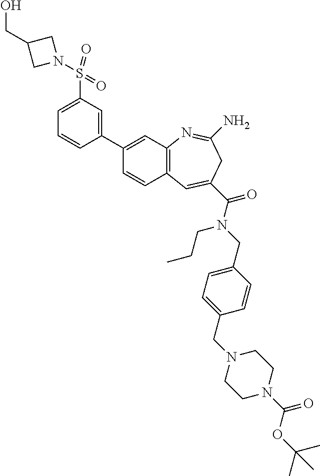 [SMILES: CCCN(Cc1ccc(CN2CCN(C(=O)OC(C)(C)C)CC2)cc1)C(=O)C1=Cc2ccc(-c3cccc(S(=O)(=O)N4CC(CO)C4)c3)cc2N=C(N)C1] | 757.0 | 9000 | 1022 |
| Bz-45 | 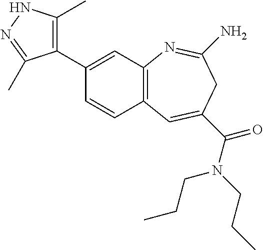 [SMILES: CCCN(CCC)C(=O)C1=Cc2ccc(-c3c(C)n[nH]c3C)cc2N=C(N)C1] | 379.5 | 9000 | 345 |
| Bz-46 | 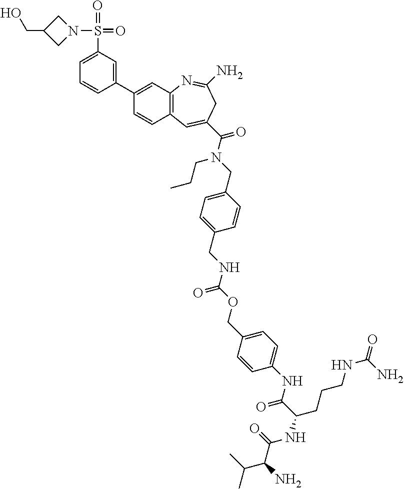 [SMILES: CCCN(Cc1ccc(CNC(=O)OCc2ccc(NC(=O)[C@H](CCCNC(N)=O)NC(=O)[C@@H](N)C(C)C)cc2)cc1)C(=O)C1=Cc2ccc(-c3cccc(S(=O)(=O)N4CC(CO)C4)c3)cc2N=C(N)C1] | 993.2 | ND | ND |
| Bz-47 | 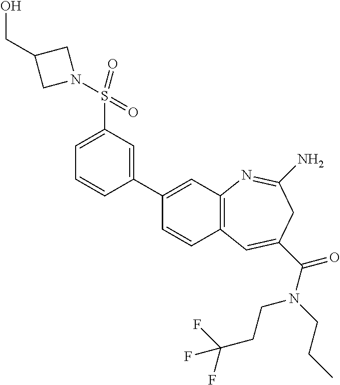 [SMILES: CCCN(CCC(F)(F)F)C(=O)C1=Cc2ccc(-c3cccc(S(=O)(=O)N4CC(CO)C4)c3)cc2N=C(N)C1] | 564.6 | 9000 | 341 |
| Bz-48 | 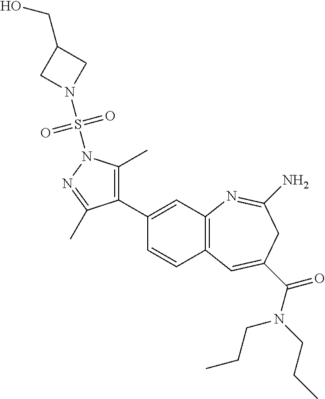 [SMILES: CCCN(CCC)C(=O)C1=Cc2ccc(-c3c(C)nn(S(=O)(=O)N4CC(CO)C4)c3C)cc2N=C(N)C1] | 528.7 | ND | 499 |
| Bz-49 | 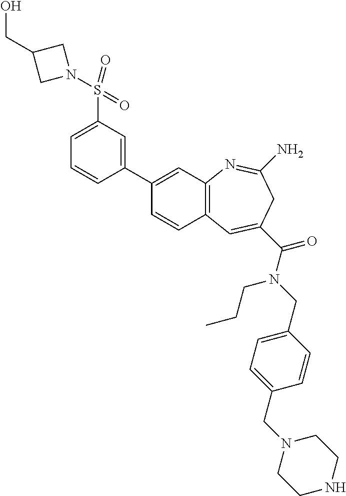 [SMILES: CCCN(Cc1ccc(CN2CCNCC2)cc1)C(=O)C1=Cc2ccc(-c3cccc(S(=O)(=O)N4CC(CO)C4)c3)cc2N=C(N)C1] | 656.8 | 9000 | 9000 |
| Bz-50 | 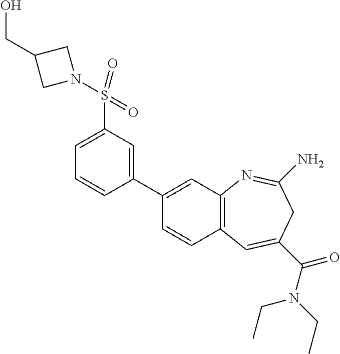 [SMILES: CCN(CC)C(=O)C1=Cc2ccc(-c3cccc(S(=O)(=O)N4CC(CO)C4)c3)cc2N=C(N)C1] | 482.6 | ND | 9000 |
| Bz-51 | 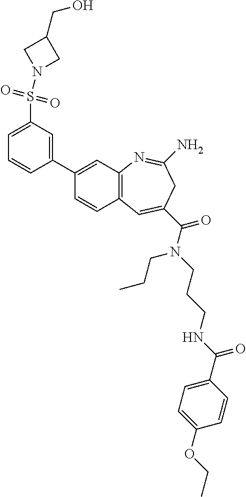 [SMILES: CCCN(CCCNC(=O)c1ccc(OCC)cc1)C(=O)C1=Cc2ccc(-c3cccc(S(=O)(=O)N4CC(CO)C4)c3)cc2N=C(N)C1] | 673.8 | 1428 | 1919 |
| Bz-52 | 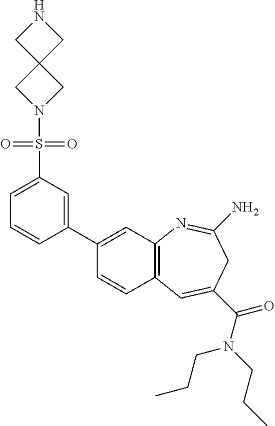 [SMILES: CCCN(CCC)C(=O)C1=Cc2ccc(-c3cccc(S(=O)(=O)N4CC5(CNC5)C4)c3)cc2N=C(N)C1] | 521.7 | ND | 1320 |
| Bz-53 | 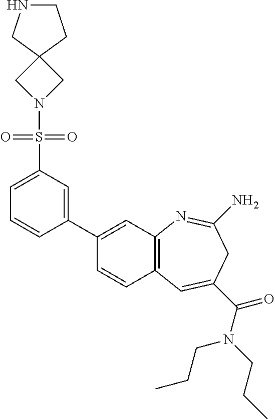 [SMILES: CCCN(CCC)C(=O)C1=Cc2ccc(-c3cccc(S(=O)(=O)N4CC5(CCNC5)C4)c3)cc2N=C(N)C1] | 535.7 | ND | 249 |
| Bz-54 | 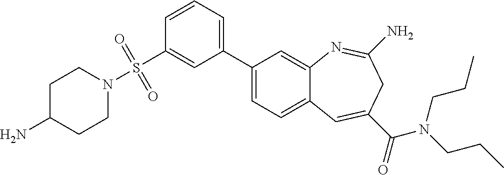 [SMILES: CCCN(CCC)C(=O)C1=Cc2ccc(-c3cccc(S(=O)(=O)N4CCC(N)CC4)c3)cc2N=C(N)C1] | 523.7 | ND | 198 |
| Bz-55 | 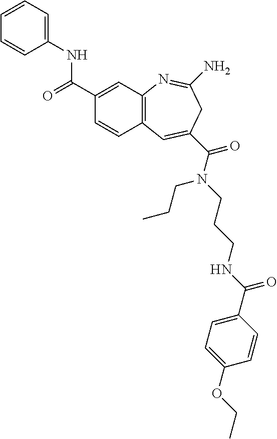 [SMILES: CCCN(CCCNC(=O)c1ccc(OCC)cc1)C(=O)C1=Cc2ccc(C(=O)Nc3ccccc3)cc2N=C(N)C1] | 567.7 | 9000 | 1040 |
| Bz-56 | 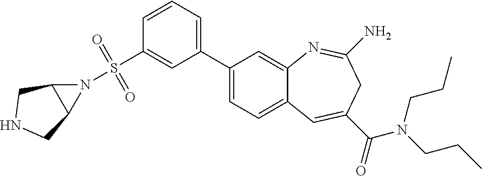 [SMILES: CCCN(CCC)C(=O)C1=Cc2ccc(-c3cccc(S(=O)(=O)N4[C@@H]5CNC[C@@H]54)c3)cc2N=C(N)C1] | 507.7 | ND | 111 |
| Bz-57 | 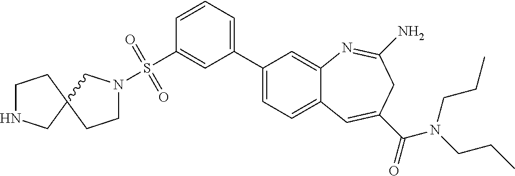 [SMILES: CCCN(CCC)C(=O)C1=Cc2ccc(-c3cccc(S(=O)(=O)N4CCC5(CCNC5)C4)c3)cc2N=C(N)C1] | 549.7 | ND | 741 |
| Bz-58 | 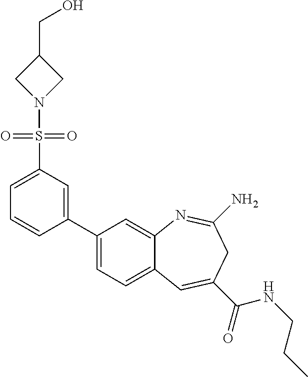 [SMILES: CCCNC(=O)C1=Cc2ccc(-c3cccc(S(=O)(=O)N4CC(CO)C4)c3)cc2N=C(N)C1] | 468.6 | 9000 | 9000 |
| Bz-59 | 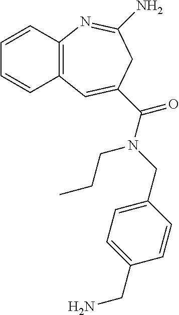 [SMILES: CCCN(Cc1ccc(CN)cc1)C(=O)C1=Cc2ccccc2N=C(N)C1] | 362.5 | 9000 | 870 |
| Bz-60 | 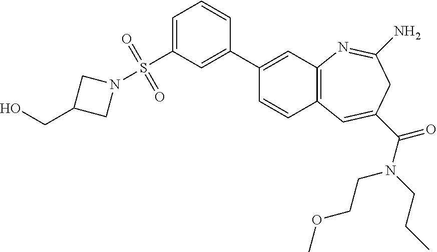 [SMILES: CCCN(CCOC)C(=O)C1=Cc2ccc(-c3cccc(S(=O)(=O)N4CC(CO)C4)c3)cc2N=C(N)C1] | 562.7 | 9000 | 288 |
| Bz-61 | 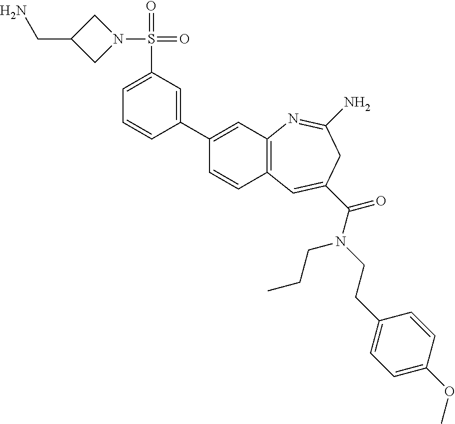 [SMILES: CCCN(CCc1ccc(OC)cc1)C(=O)C1=Cc2ccc(-c3cccc(S(=O)(=O)N4CC(CN)C4)c3)cc2N=C(N)C1] | 601.8 | 9000 | 5846 |
| Bz-62 | 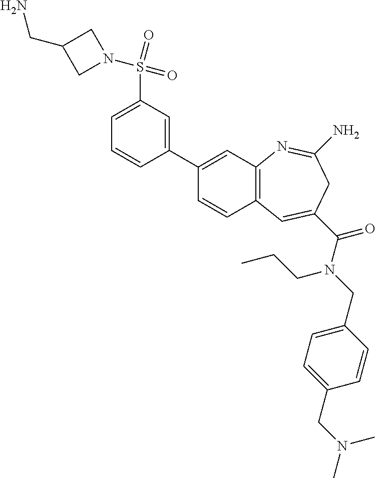 [SMILES: CCCN(Cc1ccc(CN(C)C)cc1)C(=O)C1=Cc2ccc(-c3cccc(S(=O)(=O)N4CC(CN)C4)c3)cc2N=C(N)C1] | 614.8 | 9000 | 9000 |
| Bz-63 | 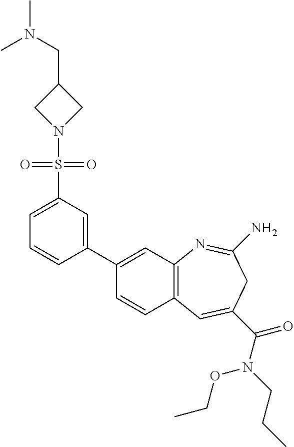 [SMILES: CCCN(OCC)C(=O)C1=Cc2ccc(-c3cccc(S(=O)(=O)N4CC(CN(C)C)C4)c3)cc2N=C(N)C1] | 539.7 | 1270 | 8 |
| Bz-64 | 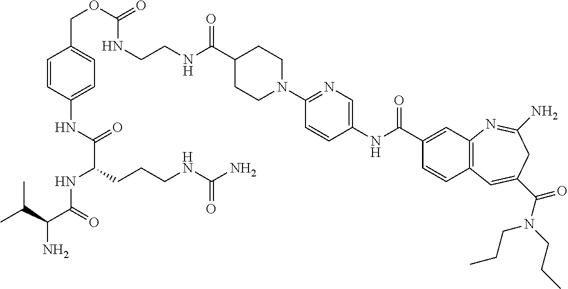 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](N)C(C)C)cc5)CC4)nc3)cc2N=C(N)C1] | 980.2 | ND | ND |
| Bz-65 | 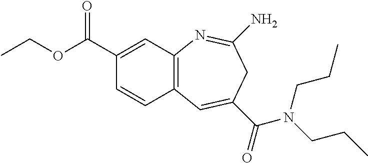 [SMILES: CCCN(CCC)C(=O)C1=Cc2ccc(C(=O)OCC)cc2N=C(N)C1] | 357.5 | 3929 | 5902 |
| Bz-66 | 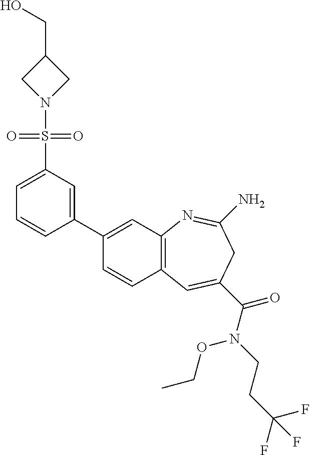 [SMILES: CCON(CCC(F)(F)F)C(=O)C1=Cc2ccc(-c3cccc(S(=O)(=O)N4CC(CO)C4)c3)cc2N=C(N)C1] | 566.6 | 4614 | 26 |
| Bz-67 | 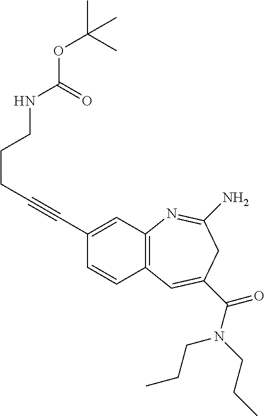 [SMILES: CCCN(CCC)C(=O)C1=Cc2ccc(C#CCCCNC(=O)OC(C)(C)C)cc2N=C(N)C1] | 466.6 | 3926 | 2053 |
| Bz-68 | 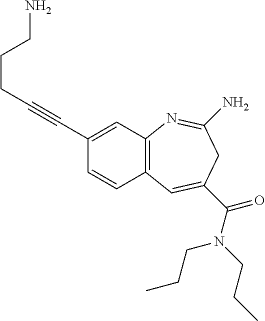 [SMILES: CCCN(CCC)C(=O)C1=Cc2ccc(C#CCCCN)cc2N=C(N)C1] | 366.5 | 4595 | 3070 |
| Bz-69 | 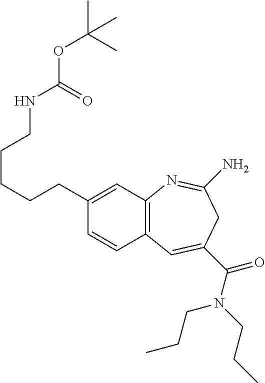 [SMILES: CCCN(CCC)C(=O)C1=Cc2ccc(CCCCCNC(=O)OC(C)(C)C)cc2N=C(N)C1] | 470.7 | 3205 | 6670 |
| Bz-70 | 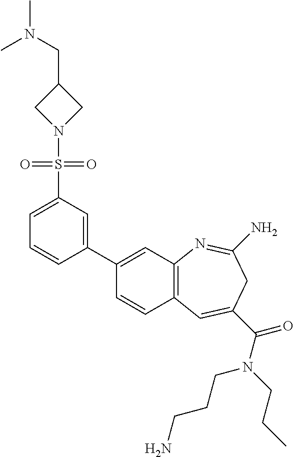 [SMILES: CCCN(CCCN)C(=O)C1=Cc2ccc(-c3cccc(S(=O)(=O)N4CC(CN(C)C)C4)c3)cc2N=C(N)C1] | 552.7 | ND | ND |
| Bz-71 | 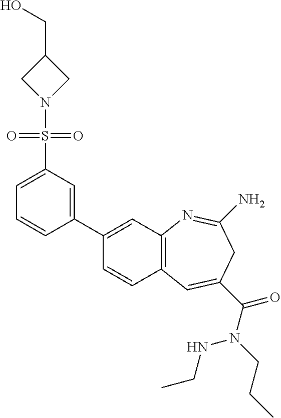 [SMILES: CCCN(NCC)C(=O)C1=Cc2ccc(-c3cccc(S(=O)(=O)N4CC(CO)C4)c3)cc2N=C(N)C1] | 511.6 | 9000 | 2752 |
| Bz-72 | 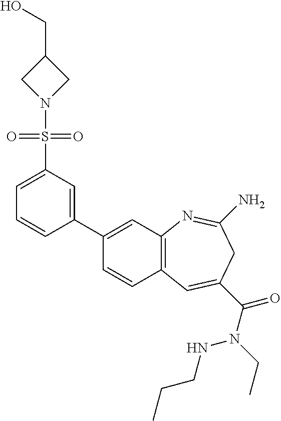 [SMILES: CCCNN(CC)C(=O)C1=Cc2ccc(-c3cccc(S(=O)(=O)N4CC(CO)C4)c3)cc2N=C(N)C1] | 511.6 | ND | 4253 |
| Bz-73 | 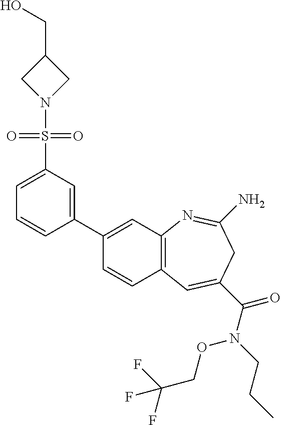 [SMILES: CCCN(OCC(F)(F)F)C(=O)C1=Cc2ccc(-c3cccc(S(=O)(=O)N4CC(CO)C4)c3)cc2N=C(N)C1] | 566.6 | 4478 | 120 |
| Bz-74 | 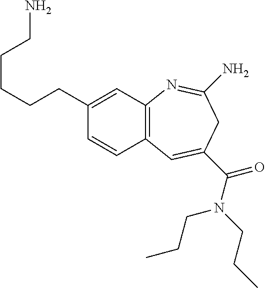 [SMILES: CCCN(CCC)C(=O)C1=Cc2ccc(CCCCCN)cc2N=C(N)C1] | 370.5 | 9000 | 2555 |
| Bz-75 | 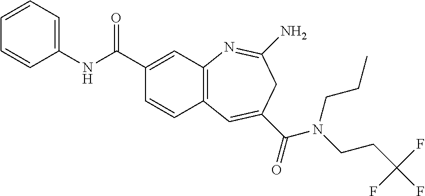 [SMILES: CCCN(CCC(F)(F)F)C(=O)C1=Cc2ccc(C(=O)Nc3ccccc3)cc2N=C(N)C1] | 458.5 | 9000 | 246 |
| Bz-76 | 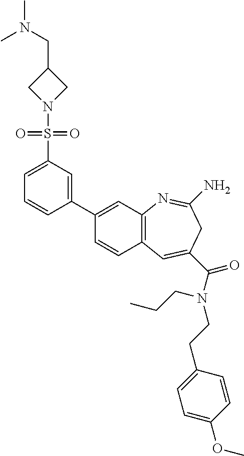 | 629.8 | 969 | 786 |
| Bz-77 | 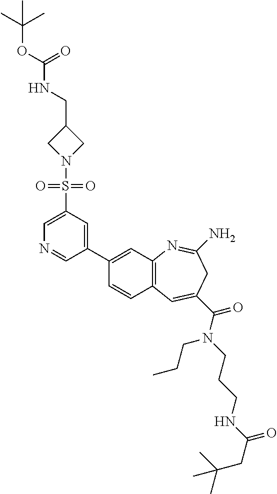 [SMILES: CCCN(CCCNC(=O)CC(C)(C)C)C(=O)C1=Cc2ccc(-c3cncc(S(=O)(=O)N4CC(CNC(=O)OC(C)(C)C)C4)c3)cc2N=C(N)C1] | 723.9 | ND | ND |
| Bz-78 | 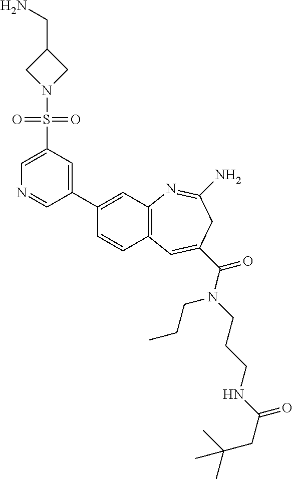 [SMILES: CCCN(CCCNC(=O)CC(C)(C)C)C(=O)C1=Cc2ccc(-c3cncc(S(=O)(=O)N4CC(CN)C4)c3)cc2N=C(N)C1] | 623.8 | ND | ND |
| Bz-79 | 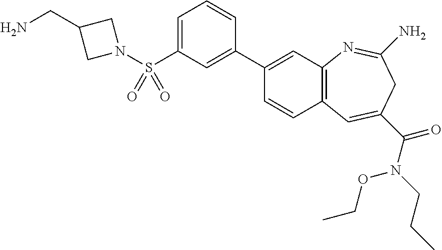 [SMILES: CCCN(OCC)C(=O)C1=Cc2ccc(-c3cccc(S(=O)(=O)N4CC(CN)C4)c3)cc2N=C(N)C1] | 511.6 | ND | ND |
| Bz-80 | 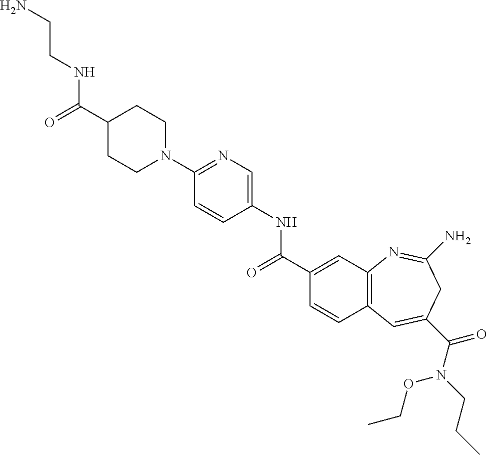 [SMILES: CCCN(OCC)C(=O)C1=Cc2ccc(C(=O)Nc3ccc(N4CCC(C(=O)NCCN)CC4)nc3)cc2N=C(N)C1] | 576.7 | ND | ND |
| Bz-81 | 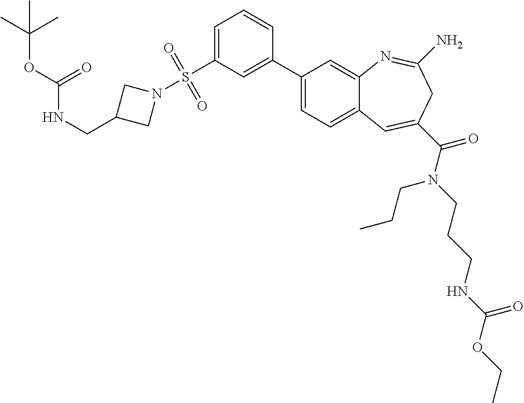 [SMILES: CCCN(CCCNC(=O)OCC)C(=O)C1=Cc2ccc(-c3cccc(S(=O)(=O)N4CC(CNC(=O)OC(C)(C)C)C4)c3)cc2N=C(N)C1] | 696.9 | ND | ND |
| Bz-82 | 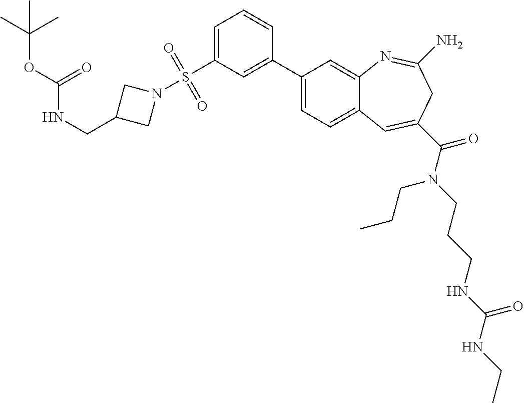 [SMILES: CCCN(CCCNC(=O)NCC)C(=O)C1=Cc2ccc(-c3cccc(S(=O)(=O)N4CC(CNC(=O)OC(C)(C)C)C4)c3)cc2N=C(N)C1] | 695.9 | ND | ND |
| Bz-83 | 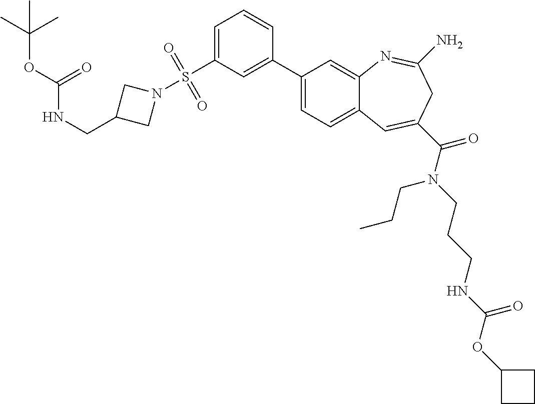 [SMILES: CCCN(CCCNC(=O)OC1CCC1)C(=O)C1=Cc2ccc(-c3cccc(S(=O)(=O)N4CC(CNC(=O)OC(C)(C)C)C4)c3)cc2N=C(N)C1] | 722.9 | ND | ND |
| Bz-84 | 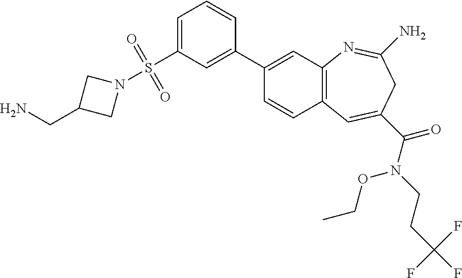 [SMILES: CCON(CCC(F)(F)F)C(=O)C1=Cc2ccc(-c3cccc(S(=O)(=O)N4CC(CN)C4)c3)cc2N=C(N)C1] | 565.6 | ND | ND |
| Bz-85 | 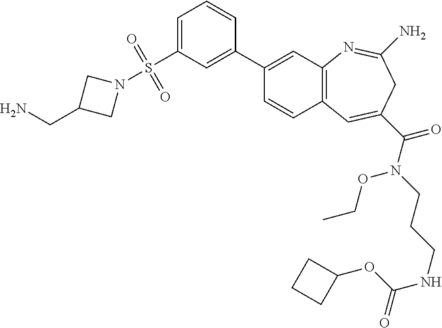 [SMILES: CCON(CCCNC(=O)OC1CCC1)C(=O)C1=Cc2ccc(-c3cccc(S(=O)(=O)N4CC(CN)C4)c3)cc2N=C(N)C1] | 624.8 | ND | ND |
| Bz-86 | 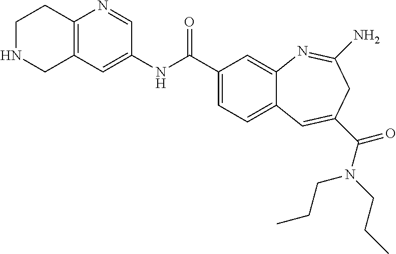 [SMILES: CCCN(CCC)C(=O)C1=Cc2ccc(C(=O)Nc3cnc4c(c3)CNCC4)cc2N=C(N)C1] | 460.6 | ND | ND |
| Bz-87 | 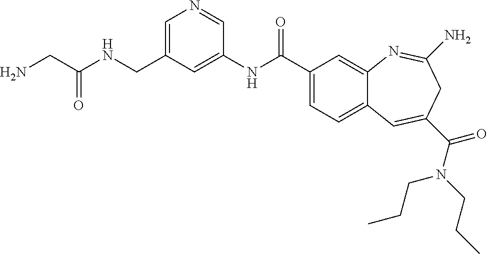 [SMILES: CCCN(CCC)C(=O)C1=Cc2ccc(C(=O)Nc3cncc(CNC(=O)CN)c3)cc2N=C(N)C1] | 491.6 | ND | ND |

### Aminobenzazepine-Linker Compounds

The immunoconjugates of the invention are prepared by conjugation of an antibody with an aminobenzazepine-linker compound. The aminobenzazepine-linker compounds comprise an aminobenzazepine moiety covalently attached to a linker unit. The linker units comprise functional groups and subunits which affect stability, permeability, solubility, and other pharmacokinetic, safety, and efficacy properties of the immunoconjugates. The linker unit includes a reactive functional group which reacts, i.e. conjugates, with a reactive functional group of the antibody. For example, a nucleophilic group such as a lysine side chain amino of the antibody reacts with an electrophilic reactive functional group of the aminobenzazepine-linker compound to form the immunoconjugate. Also, for example, a cysteine thiol of the antibody reacts with a maleimide or bromoacetamide group of the aminobenzazepine-linker compound to form the immunoconjugate.

Electrophilic reactive functional group suitable for the aminobenzazepine-linker compounds include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2nd Edition, Academic Press, 2008.

The invention provides solutions to the limitations and challenges to the design, preparation and use of immunoconjugates. Some linkers may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases immunoconjugate yield and can render process scale-up difficult.

Exemplary embodiments include an aminobenzazepine-linker compound of Formula II:

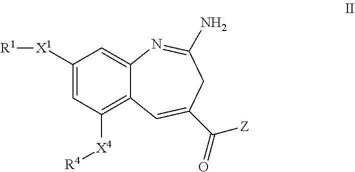

wherein
Z is selected from H, —O(C₁-C₅ alkyl), and N(X²R²)(X³R³);
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-NR⁵—C(═NR^{5a})NR⁵—*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—C(═O)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₅ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)—(C₂-C₅ heteroaryl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring;
X¹, X², X³, and X⁴ are independently selected from the group consisting of a bond, C(═O), C(═O)N(R⁵), 0, N(R⁵), S, S(O)₂, and S(O)₂N(R⁵);
R⁵ is selected from the group consisting of H, C₆-C₂₀ aryl, C₆-C₂₀ aryldiyl, C₁-C₁₂ alkyl, and C₁-C₁₂ alkyldiyl, or two R⁵ groups together form a 5- or 6-membered heterocyclyl ring;
R⁵, is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
Q-C(═O)-(PEG)-;
Q-C(═O)-(PEG)-C(═O)—;
Q-C(═O)-(PEG)-O—;
Q-C(═O)-(PEG)-C(═O)-(PEP)-;
Q-C(═O)-(PEG)-C(═O)N(R⁵)—(C₁-C₁₂ alkyldiyl)-;
Q-C(═O)-(PEG)-C(═O)N(R⁵)—(C₁-C₁₂ alkyldiyl)-N(R⁵)C(═O)—(C₂-C₅ monoheterocyclyldiyl)-;
Q-C(═O)-(PEG)-C(═O)N(R⁵)—(C₁-C₁₂ alkyldiyl)-(MCgluc)-;
Q-C(═O)-(PEG)-C(═O)-(MCgluc)-;
Q-C(═O)-(PEG)-C(═O)-(PEP)-N(R⁵)—(C₁-C₁₂ alkyldiyl)-;
Q-C(═O)-(PEG)-C(═O)-(PEP)-N(R⁵)—(C₁-C₁₂ alkyldiyl)-N(R⁵)C(═O)—(C₂-C₅ monoheterocyclyldiyl)-;
Q-C(═O)-(PEG)-N(R⁵)—;
Q-C(═O)-(PEG)-N(R⁵)—(PEG)-C(═O)-(PEP)-;
Q-C(═O)-(PEG)-N⁺(R⁵)₂-(PEG)-C(═O)-(PEP)-;
Q-C(═O)-(PEG)-C(═O)—N(R⁵)CH(AA₁)C(═O)-(PEG)-C(═O)-(PEP)-;
Q-C(═O)-(PEG)-C(═O)—N(R⁵)CH(AA₁)C(═O)—N(R⁵)—(C₁-C₁₂ alkyldiyl)-;
Q-C(═O)-(PEG)-SS—(C₁-C₁₂ alkyldiyl)-OC(═O)—;
Q-C(═O)-(PEG)-SS—(C₁-C₁₂ alkyldiyl)-C(═O)—;
Q-C(═O)—(C₁-C₁₂ alkyldiyl)-C(═O)-(PEP)-;
Q-C(═O)—(C₁-C₁₂ alkyldiyl)-C(═O)-(PEP)-N(R⁵)—(C₁-C₁₂ alkyldiyl)-;
Q-C(═O)—(C₁-C₁₂ alkyldiyl)-C(═O)-(PEP)-N(R⁵)—(C₁-C₁₂ alkyldiyl)-N(R⁵)—C(═O);
Q-C(═O)—(C₁-C₁₂ alkyldiyl)-C(═O)-(PEP)-N(R⁵)—(C₁-C₁₂ alkyldiyl)-N(R⁵)C(═O)—(C₂-C₅ monoheterocyclyldiyl)-;
Q-C(═O)—CH₂CH₂OCH₂CH₂—(C₁-C₂₀ heteroaryldiyl)-CH₂O—(PEG)-C(═O)-(MCgluc)-;
Q-C(═O)—CH₂CH₂OCH₂CH₂—(C₁-C₂₀ heteroaryldiyl)-CH₂O—(PEG)-C(═O)-(MCgluc)-N(R⁵)—(C₁-C₁₂ alkyldiyl)-N(R⁵)C(═O)—(C₂-C₅ monoheterocyclyldiyl)-; and
Q-(CH₂)ₘ—C(═O)-(PEP)-N(R⁵)—(C₁-C₁₂ alkyldiyl)-N(R⁵)C(═O)—(C₂-C₅ monoheterocyclyldiyl)-;
where PEG has the formula:—(CH₂CH₂O)ₙ—(CH₂)ₘ—; m is an integer from 1 to 5, and n is an integer from 2 to 50;
PEP has the formula:

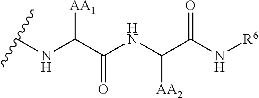

where AA₁ and AA₂ are independently selected from an amino acid side chain, or AA₁ or AA₂ and an adjacent nitrogen atom form a 5-membered ring proline amino acid, and the wavy line indicates a point of attachment and;
R⁶ is selected from the group consisting of C₆-C₂₀ aryldiyl and C₁-C₂₀ heteroaryldiyl, substituted with —CH₂O—C(═O)— and optionally with:

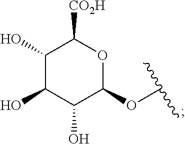 [SMILES: *O[C@@H]1O[C@H](C(=O)O)[C@@H](O)[C@H](O)[C@H]1O]

and
MCgluc is selected from the groups:

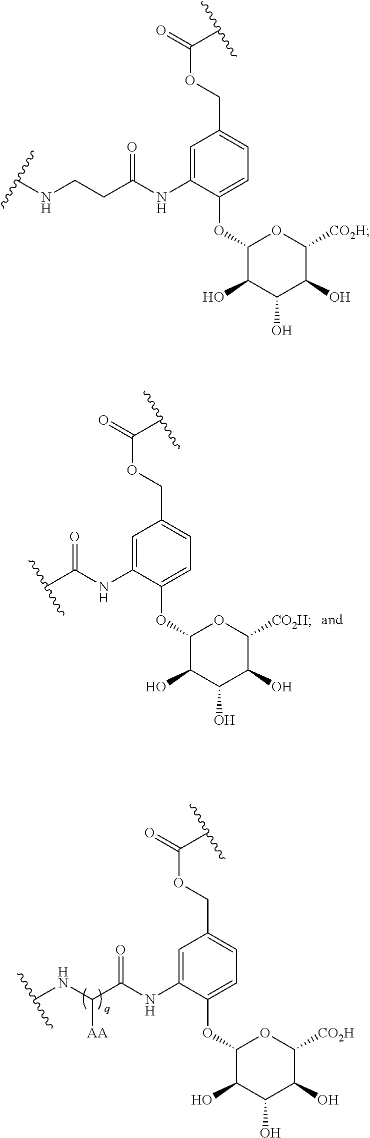

where q is 1 to 8, and AA is an amino acid side chain; and
Q is selected from the group consisting of N-hydroxysuccinimidyl, N-hydroxysulfosuccinimidyl, maleimide, and phenoxy substituted with one or more groups independently selected from F, Cl, NO₂, and SO₃⁻;
where alkyl, alkyldiyl, alkenyl, alkenyldiyl, alkynyl, alkynyldiyl, aryl, aryldiyl carbocyclyl, carbocyclyldiyl, heterocyclyl, heterocyclyldiyl, heteroaryl, and heteroaryldiyl are optionally substituted with one or more groups independently selected from F, Cl, Br, I, —CN, —CH₃, —CH₂CH₃, —CH═CH₂, —C═CH, —C═CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)˜H, —OP(O)(OH)₂, —S(O)₂N(CH₃)₂, —SCH₃, —S(O)₂CH₃, and —S(O)₃H.

An exemplary embodiment of the aminobenzazepine-linker compound of Formula II includes wherein PEP is selected from the groups:

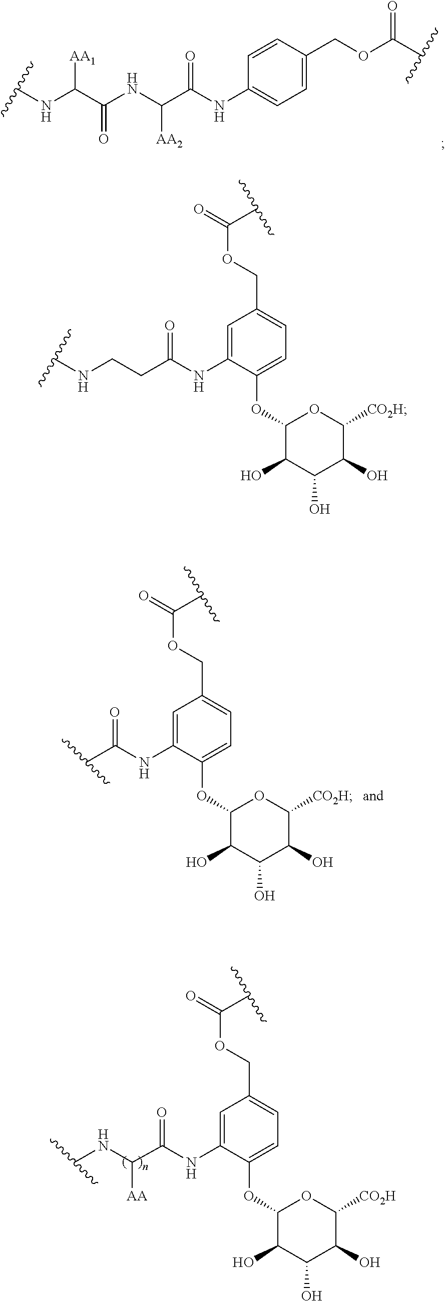

where n is 1 or more, and AA is an amino acid side chain.

An exemplary embodiment of the aminobenzazepine-linker compound of Formula II includes wherein AA₁ and AA₂ are independently selected from a side chain of a naturally-occurring amino acid.

An exemplary embodiment of the aminobenzazepine-linker compound of Formula II includes wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CH₂CH(CH₃)₂, —CH₂SO₃H, and —CH₂CH₂CH₂NHC(O)NH₂.

An exemplary embodiment of the aminobenzazepine-linker compound of Formula II includes wherein AA₁ is —CH(CH₃)₂, and AA₂ is —CH₂CH₂CH₂NHC(O)NH₂.

An exemplary embodiment of the aminobenzazepine-linker compound of Formula II includes wherein AA₁ and AA₂ are independently selected from GlcNAc aspartic acid, —CH₂SO₃H, and —CH₂OPO₃H.

An exemplary embodiment of the aminobenzazepine-linker compound of Formula II is selected from Formulas IIa-d:

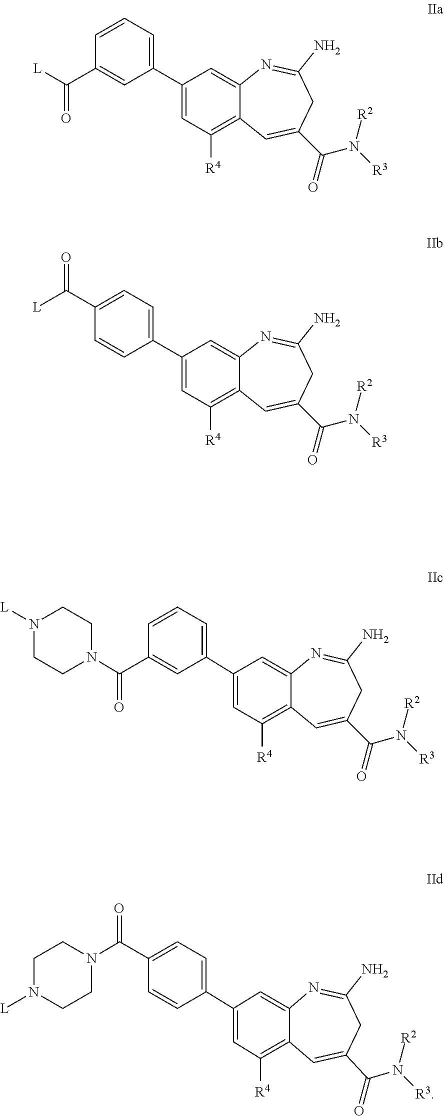

An exemplary embodiment of the aminobenzazepine-linker compound of Formula II is selected from Formulas IIe and IIf:

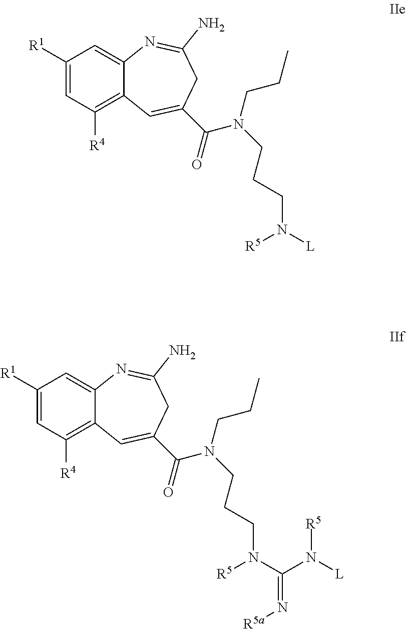

where R^{5a} of formula IIf is phenyl, optionally substituted with one or more groups selected from F, Cl, Br, I, —CN, and —NO₂.

An exemplary embodiment of the aminobenzazepine-linker compound of Formula II includes wherein L is Q-C(═O)-(PEG)- or Q-C(═O)-(PEG)-C(═O)—.

An exemplary embodiment of the aminobenzazepine-linker compound of Formula II is selected from Formulas IIg and IIh.

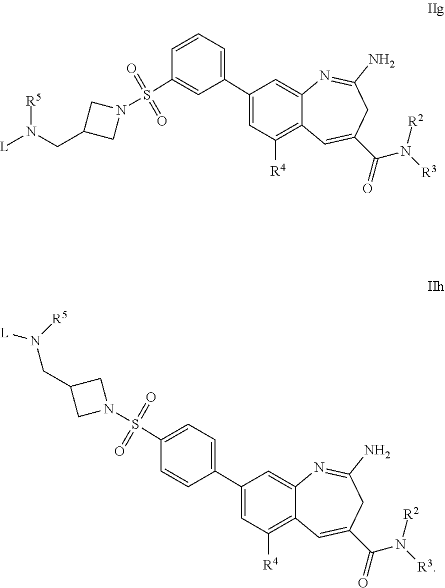

An exemplary embodiment of the aminobenzazepine-linker compound of Formula II includes wherein L is —C(═O)-(PEG)-C(═O)-(PEP)-.

An exemplary embodiment of the aminobenzazepine-linker compound of Formula II includes wherein R² and R³ are each C₁-C₈alkyl.

An exemplary embodiment of the aminobenzazepine-linker compound of Formula II includes wherein R² and R³ are each —CH₂CH₂CH₃.

An exemplary embodiment of the aminobenzazepine-linker compound of Formula II includes wherein X² and X³ are each a bond, and R² or R³ is —O—(C₁-C₁₂ alkyl).

An exemplary embodiment of the aminobenzazepine-linker compound of Formula II includes wherein X² and X³ are each a bond, and R² or R³ is —OCH₂CH₃.

An exemplary embodiment of the aminobenzazepine-linker compound of Formula II includes wherein one of R¹ and R⁴ is selected from —(C₆-C₂ aryldiyl)-S(═O)₂—(C₂-C₂₀ heterocyclyldiyl)-(C₁-C₁₂ alkyldiyl)-N(R⁵)₂ and —(C₆-C₂₀ aryldiyl)-S(═O)₂—(C₂-C₂₀ heterocyclyldiyl)-(C₁-C₁₂ alkyldiyl)-OH.

An exemplary embodiment of the aminobenzazepine-linker compound of Formula II includes wherein C₆-C₂₀ aryldiyl is phenyldiyl and C₂-C₂₀ heterocyclyldiyl is azetidindiyl.

An exemplary embodiment of the aminobenzazepine-linker compound of Formula II is selected from the formulas.

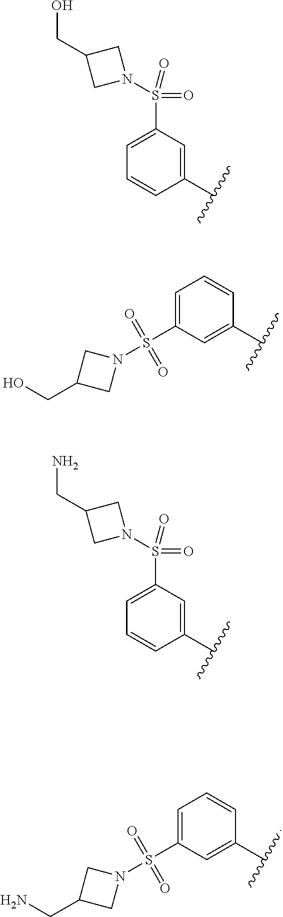

An exemplary embodiment of the aminobenzazepine-linker compound of Formula II includes wherein one of R¹ and R⁴ is —C(═O)NR⁵—(C₁-C₂₀ heteroaryldiyl)-(C₂-C₂O heterocyclyldiyl)-C(═O)NR⁵—(C₁-C₁₂ alkyldiyl)-NR⁵-L.

An exemplary embodiment of the aminobenzazepine-linker compound of Formula II includes wherein C₁-C₂₀ heteroaryldiyl is pyridindiyl and C₂-C₂₀ heterocyclyldiyl is piperidiyl.

An exemplary embodiment of the aminobenzazepine-linker compound of Formula II includes wherein Q is selected from:

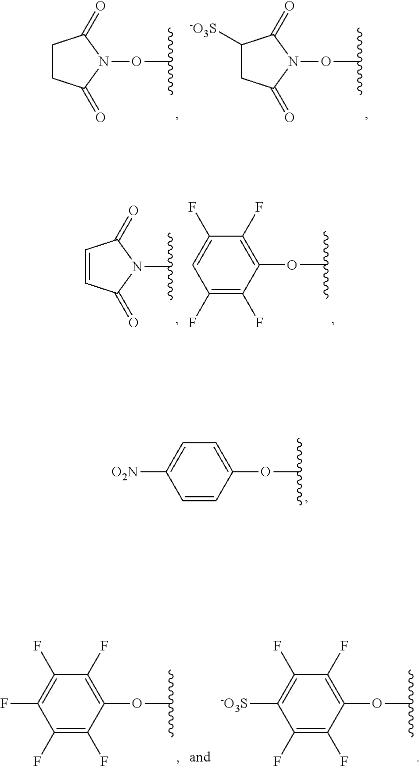

The invention includes all reasonable combinations, and permutations of the features, of the Formula II embodiments.

An exemplary embodiment of the aminobenzazepine-linker compound of Formula II is selected from the Table 2a, 2b, and 2c compounds. Each compound was synthesized and purified by the methods in the Examples provided herein, characterized by mass spectrometry, and shown to have the mass indicated. The aminobenzazepine-linker compounds of Tables 2a, 2b, and 2c demonstrate the surprising and unexpected property of TLR8 agonist selectivity which may predict useful therapeutic activity to treat cancer and other disorders.

TABLE 2a
Aminobenzazepine-linker Formula II compounds (BzL) and intermediates

| BzL | | |
|---|---|---|
| No. | Structure | MW |
| BzL-1 | 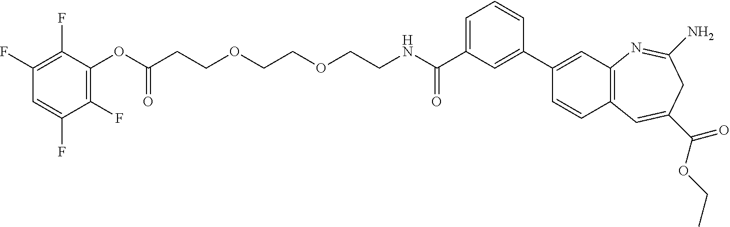 [SMILES: CCOC(=O)C1=Cc2ccc(-c3cccc(C(=O)NCCOCCOCCC(=O)Oc4c(F)c(F)cc(F)c4F)c3)cc2N=C(N)C1] | 657.6 |
| BzL-2 | 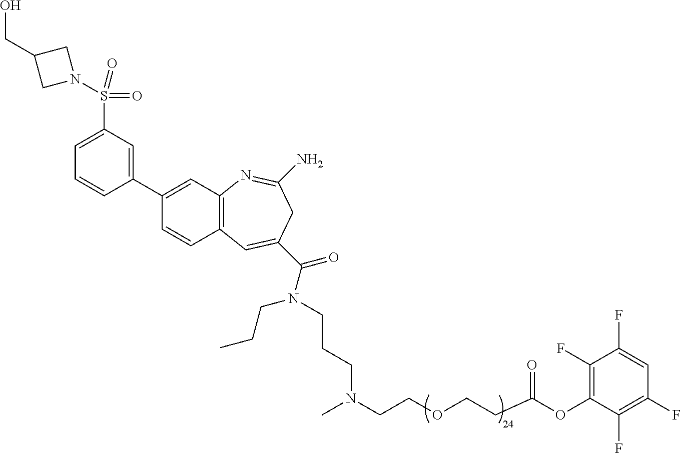 | 1817.1 |
| BzL-3 | 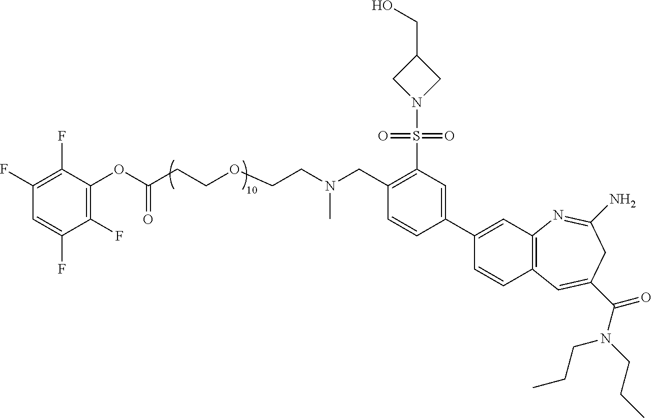 | 1214.4 |
| BzL-4 | 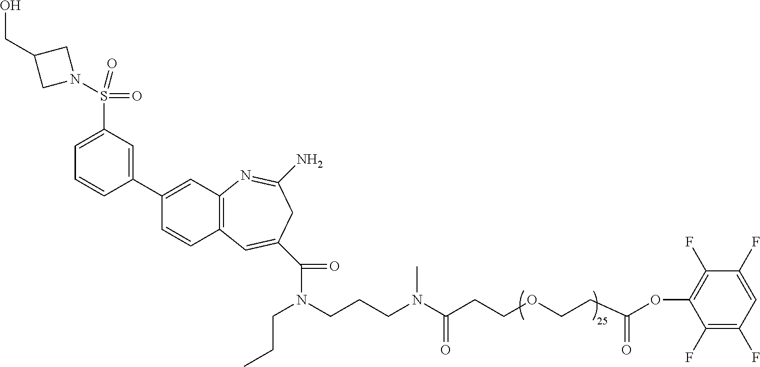 | 1889.1 |
| BzL-5 | 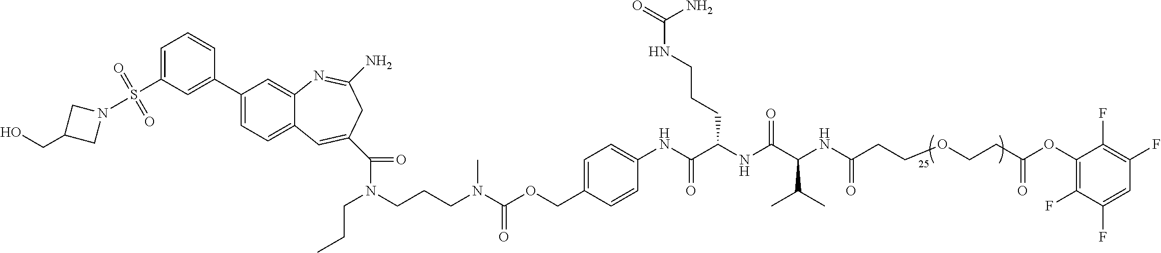 | 2294.6 |
| BzL-6 | 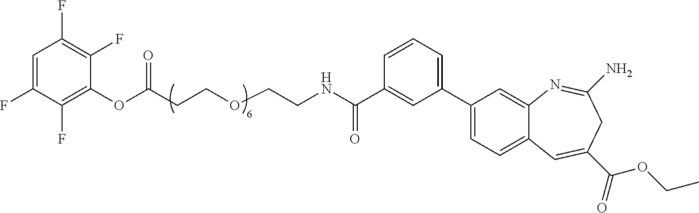 | 833.82 |
| BzL-7 | 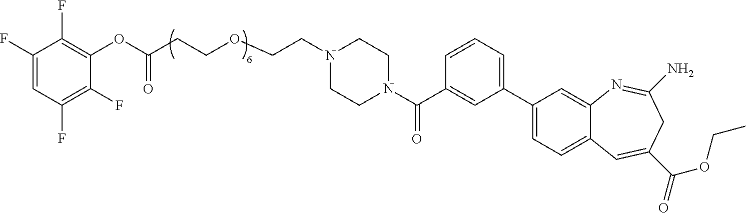 | 902.9 |
| BzL-8 | 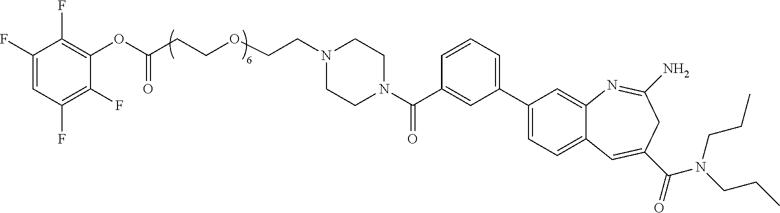 | 958.1 |
| BzL-9 | 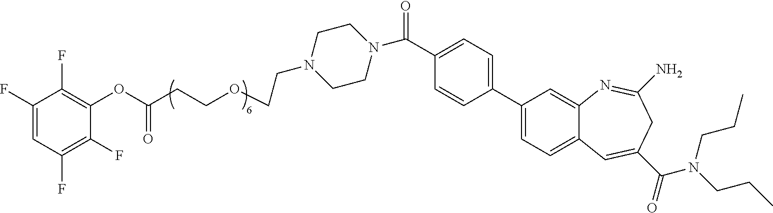 | 958.1 |
| BzL-10 | 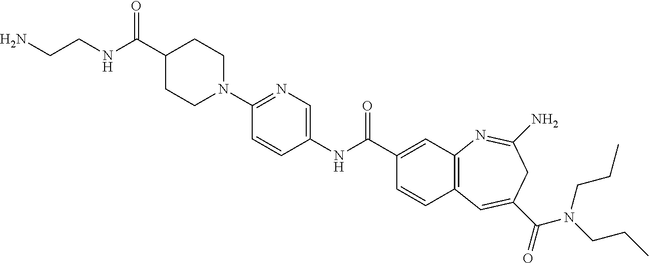 [SMILES: CCCN(CCC)C(=O)C1=Cc2ccc(C(=O)Nc3ccc(N4CCC(C(=O)NCCN)CC4)nc3)cc2N=C(N)C1] | 574.7 |
| BzL-11 | 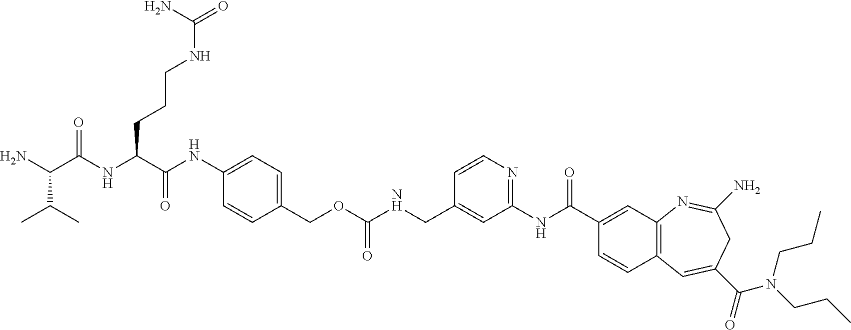 [SMILES: CCCN(CCC)C(=O)C1=Cc2ccc(C(=O)Nc3cc(CNC(=O)OCc4ccc(NC(=O)[C@H](CCCNC(N)=O)NC(=O)[C@@H](N)C(C)C)cc4)ccn3)cc2N=C(N)C1] | 840.0 |
| BzL-12 | 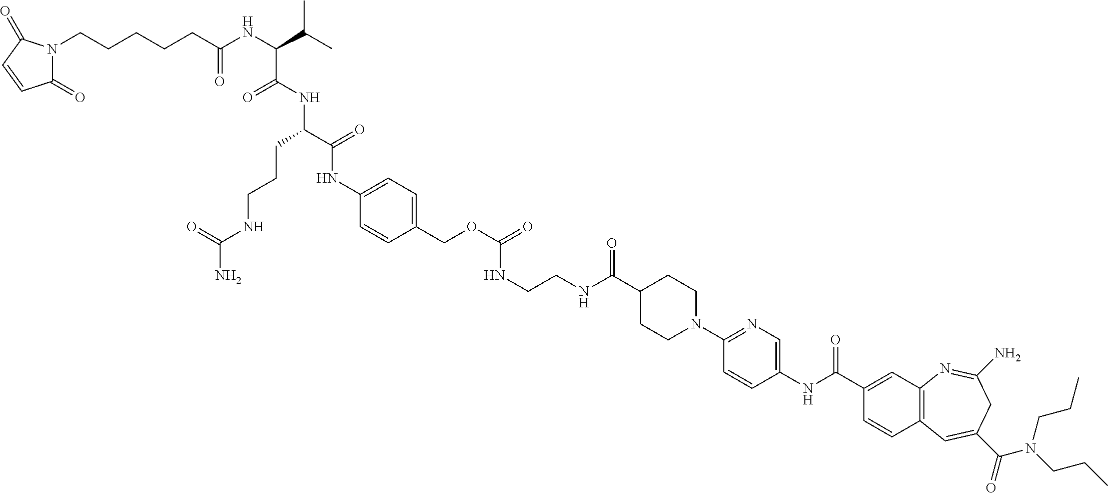 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1] | 1173.4 |
| BzL-13 | 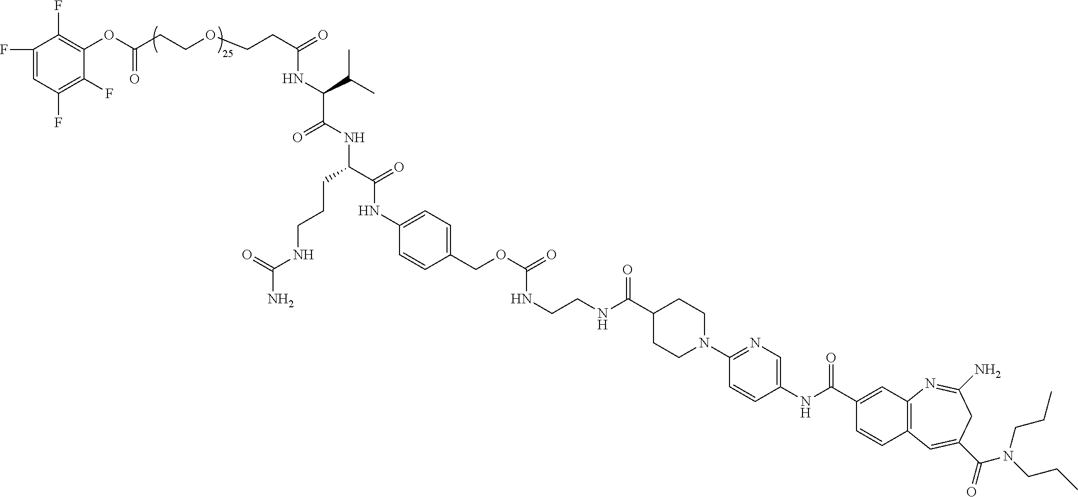 | 2329.6 |
| BzL-14 | 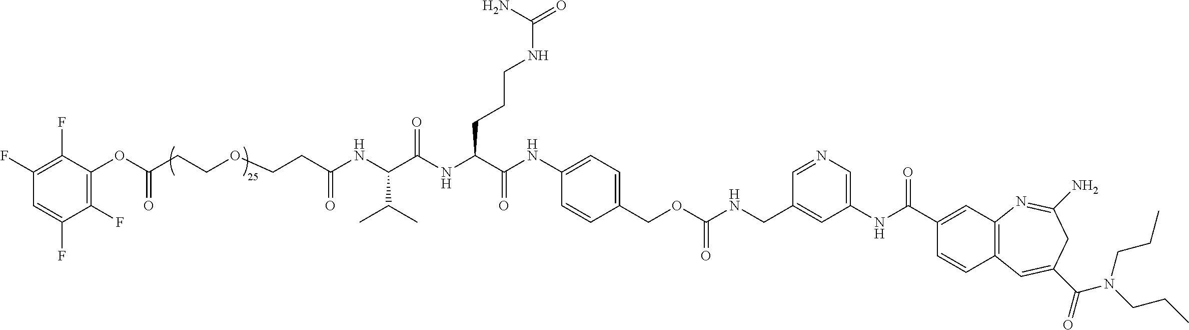 | 2189.4 |
| BzL-15 | 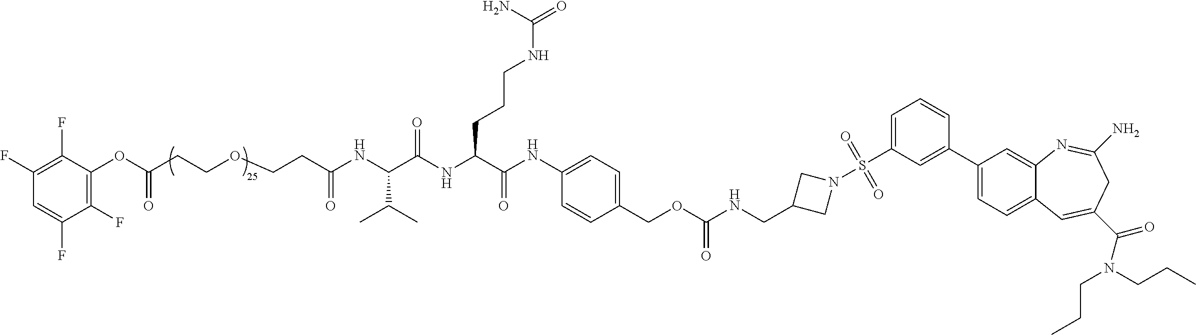 | 2264.6 |
| BzL-16 | 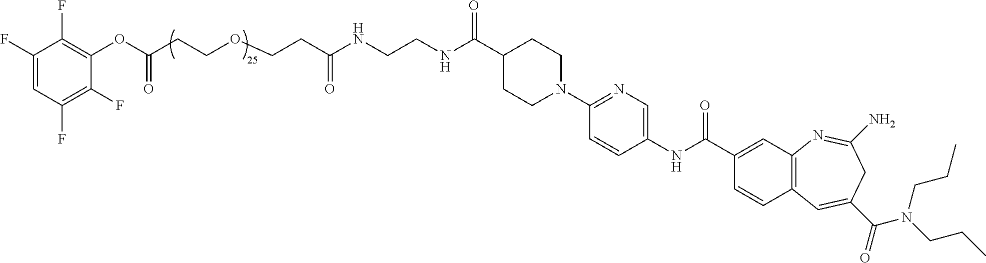 | 1924.2 |
| BzL-17 | 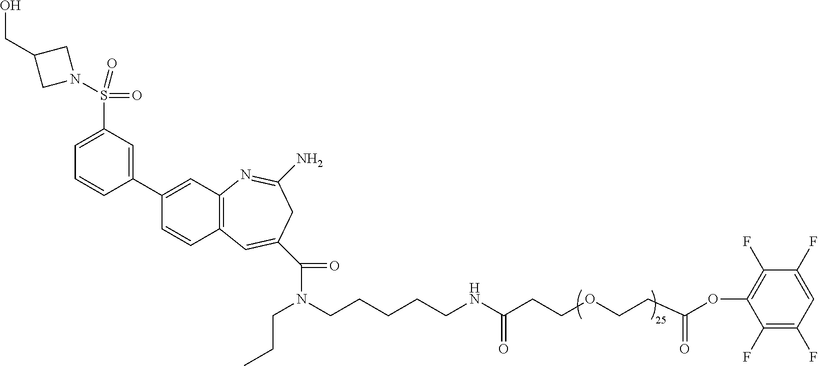 | 1903.2 |
| BzL-18 | 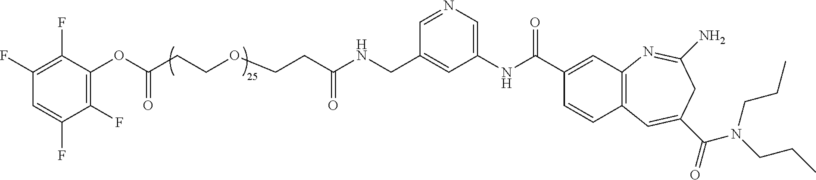 | 1784 |
| BzL-19 | 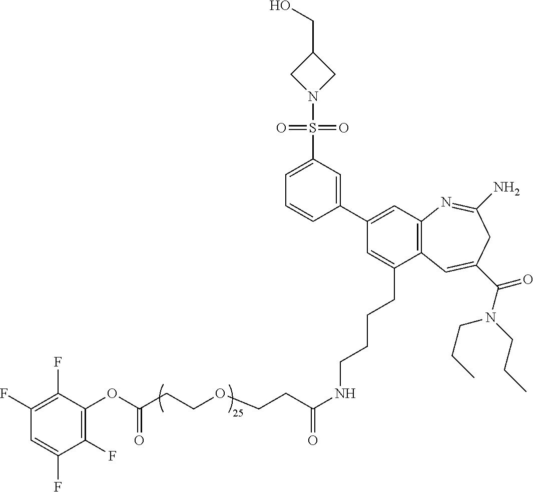 | 1931.2 |
| BzL-20 | 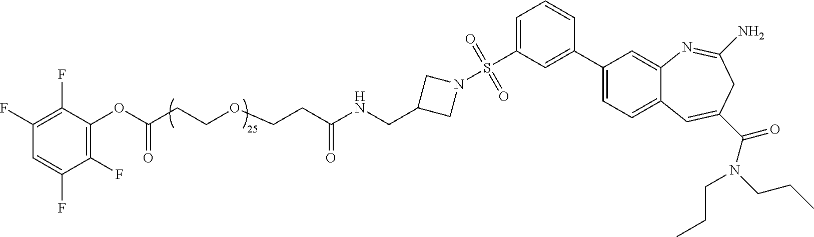 | 1859.1 |
| BzL-21 | 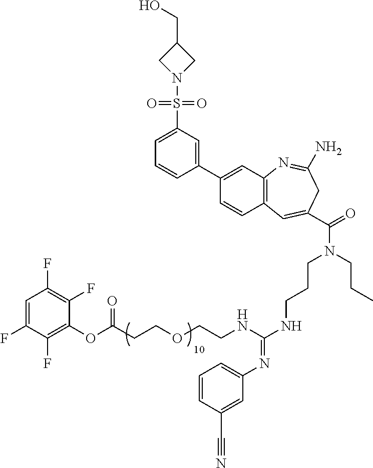 | 1329.5 |
| BzL-22 | 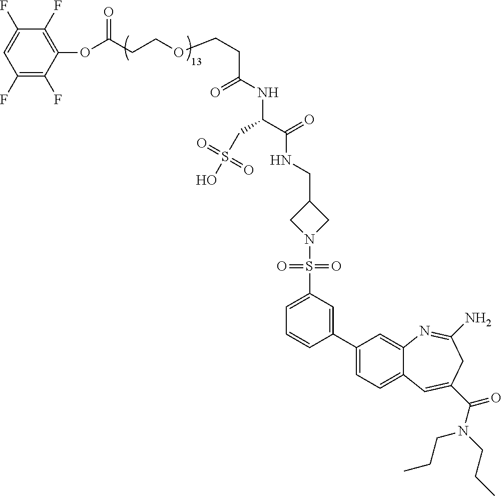 | 1481.6 |
| BzL-23 | 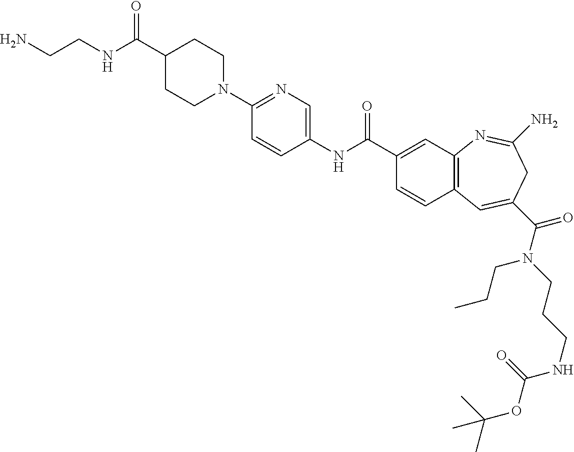 [SMILES: CCCN(CCCNC(=O)OC(C)(C)C)C(=O)C1=Cc2ccc(C(=O)Nc3ccc(N4CCC(C(=O)NCCN)CC4)nc3)cc2N=C(N)C1] | 689.9 |
| BzL-24 | 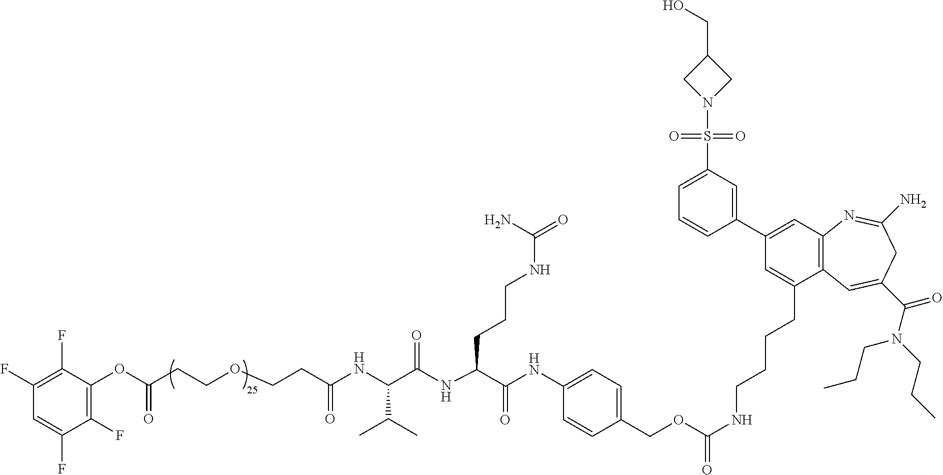 | 2336.7 |
| BzL-25 | 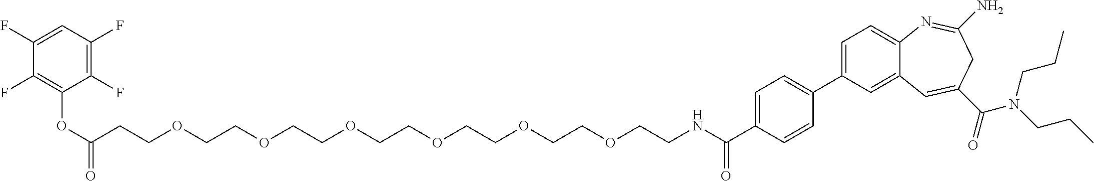 [SMILES: CCCN(CCC)C(=O)C1=Cc2cc(-c3ccc(C(=O)NCCOCCOCCOCCOCCOCCOCCC(=O)Oc4c(F)c(F)cc(F)c4F)cc3)ccc2N=C(N)C1] | 888.95 |
| BzL-26 | 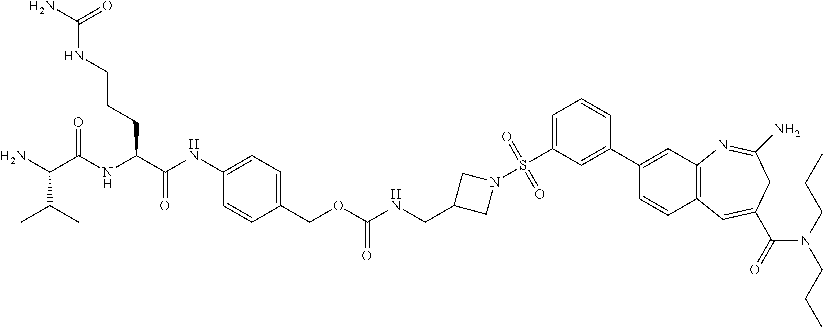 [SMILES: CCCN(CCC)C(=O)C1=Cc2ccc(-c3cccc(S(=O)(=O)N4CC(CNC(=O)OCc5ccc(NC(=O)[C@H](CCCNC(N)=O)NC(=O)[C@@H](N)C(C)C)cc5)C4)c3)cc2N=C(N)C1] | 915.1 |
| BzL-27 | 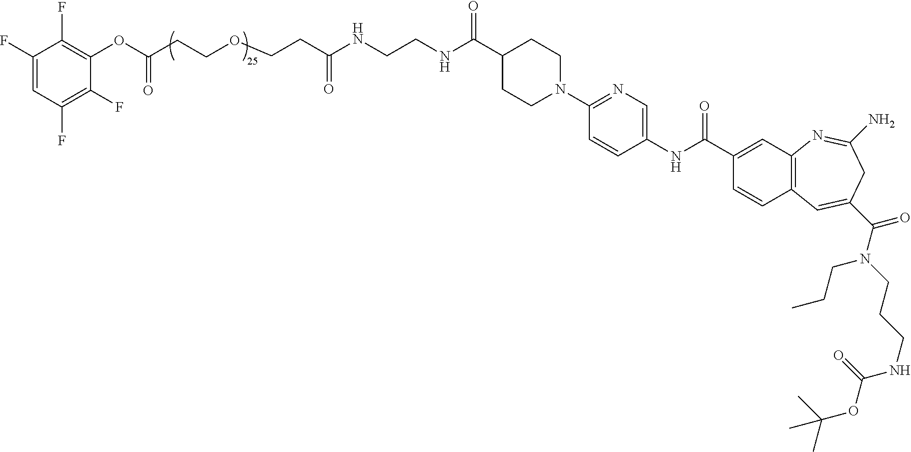 | 2039.3 |
| BzL-28 | 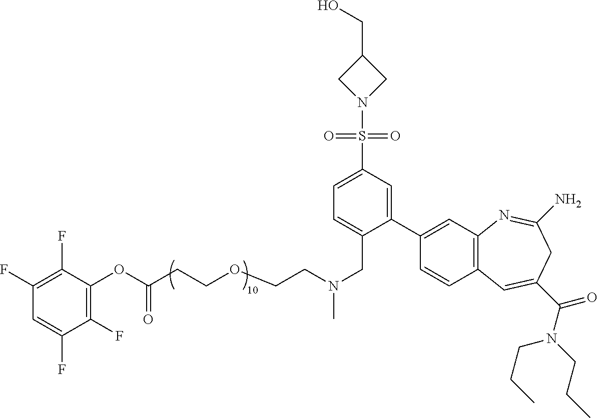 | 1214.4 |
| BzL-29 | 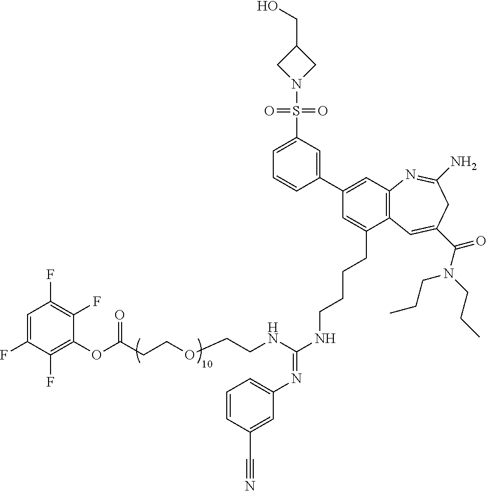 | 1385.6 |
| BzL-30 | 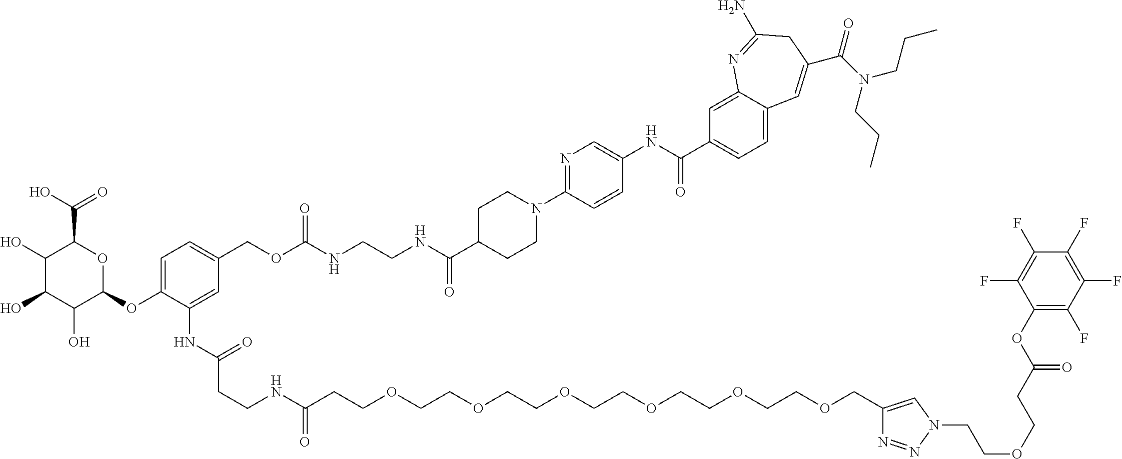 [SMILES: CCCN(CCC)C(=O)C1=Cc2ccc(C(=O)Nc3ccc(N4CCC(C(=O)NCCNC(=O)OCc5ccc(O[C@@H]6O[C@H](C(=O)O)C(O)[C@H](O)C6O)c(NC(=O)CCNC(=O)CCOCCOCCOCCOCCOCCOCc6cn(CCOCCC(=O)Oc7c(F)c(F)c(F)c(F)c7F)nn6)c5)CC4)nc3)cc2N=C(N)C1] | 1642.6 |
| BzL-31 | 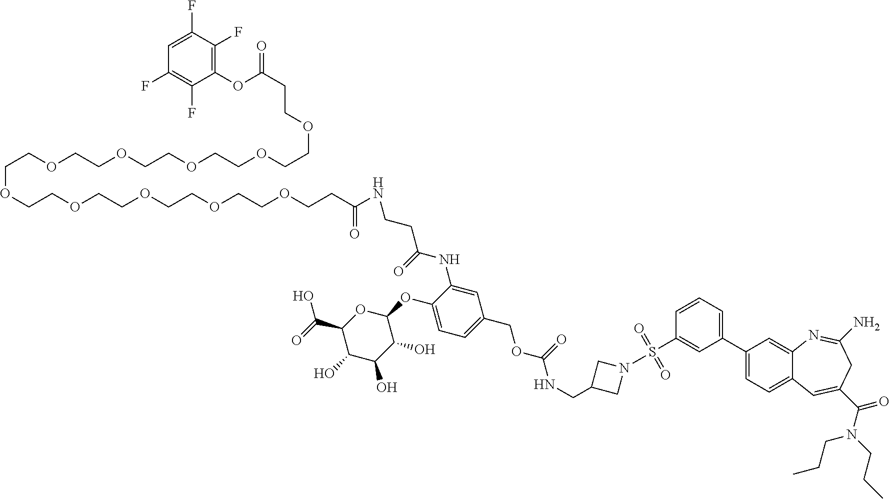 [SMILES: CCCN(CCC)C(=O)C1=Cc2ccc(-c3cccc(S(=O)(=O)N4CC(CNC(=O)OCc5ccc(O[C@@H]6O[C@H](C(=O)O)[C@@H](O)[C@H](O)[C@H]6O)c(NC(=O)CCNC(=O)CCOCCOCCOCCOCCOCCOCCOCCOCCOCCOCCC(=O)Oc6c(F)c(F)cc(F)c6F)c5)C4)c3)cc2N=C(N)C1] | 1610.7 |
| BzL-32 | 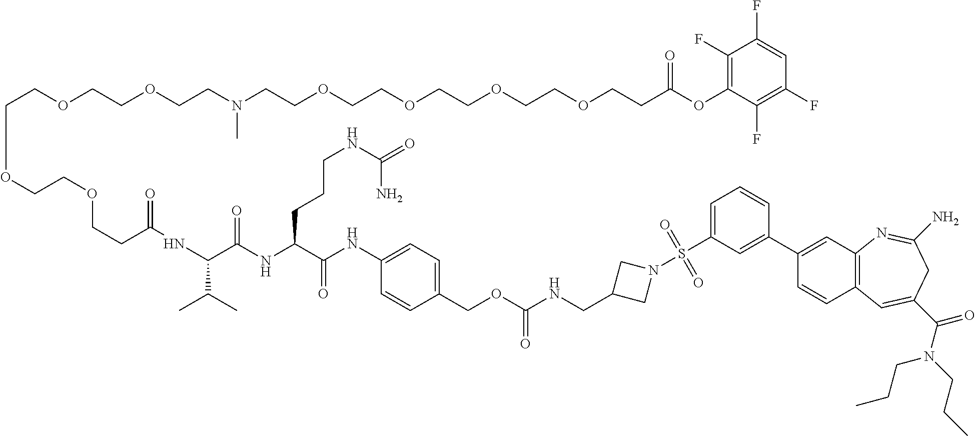 [SMILES: CCCN(CCC)C(=O)C1=Cc2ccc(-c3cccc(S(=O)(=O)N4CC(CNC(=O)OCc5ccc(NC(=O)[C@H](CCCNC(N)=O)NC(=O)[C@@H](NC(=O)CCOCCOCCOCCOCCN(C)CCOCCOCCOCCOCCC(=O)Oc6c(F)c(F)cc(F)c6F)C(C)C)cc5)C4)c3)cc2N=C(N)C1] | 1572.8 |

TABLE 2b
Aminobenzazepine-linker Formula II compounds (BzL) and intermediates

| BzL | | |
|---|---|---|
| No. | Structure | MW |
| BzL-33 | 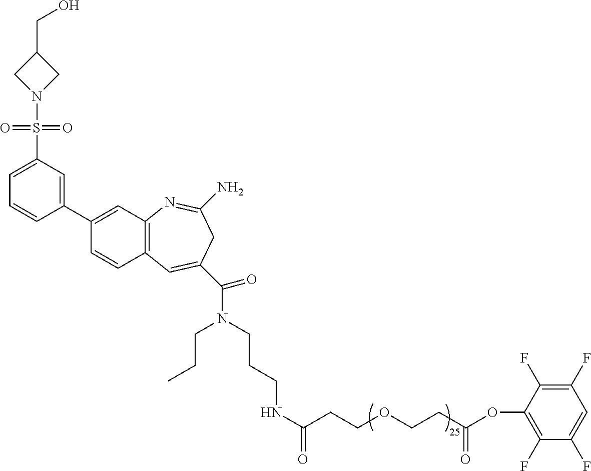 | 1875.1 |
| BzL-34 | 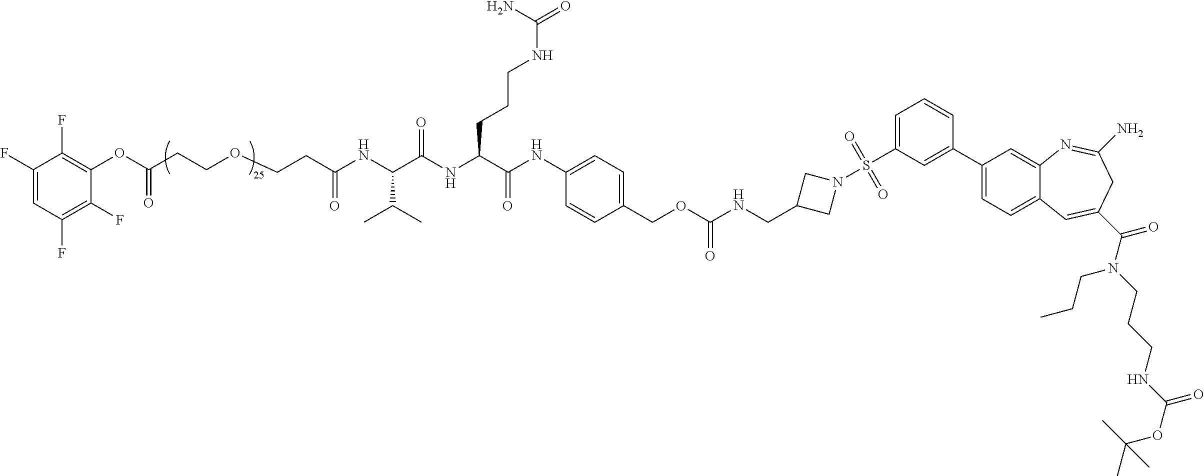 | 2379.7 |
| BzL-35 | 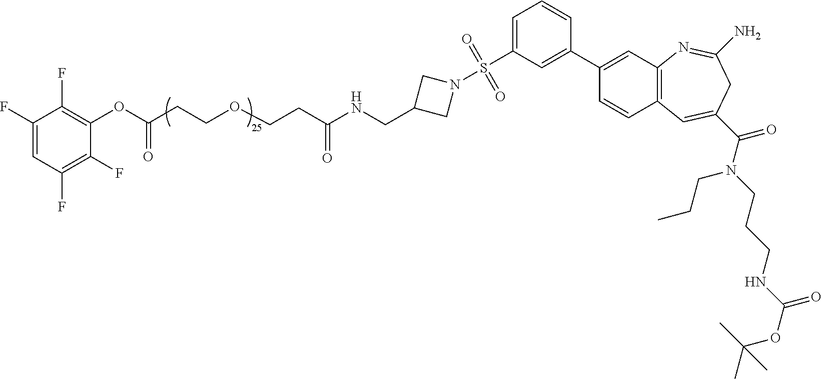 | 1974.2 |
| BzL-36 | 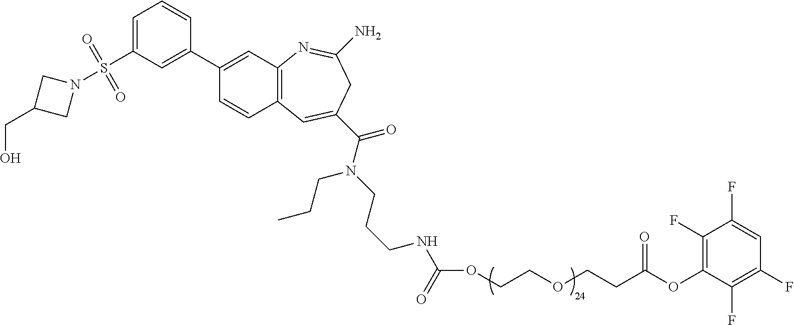 | 1847.1 |
| BzL-37 | 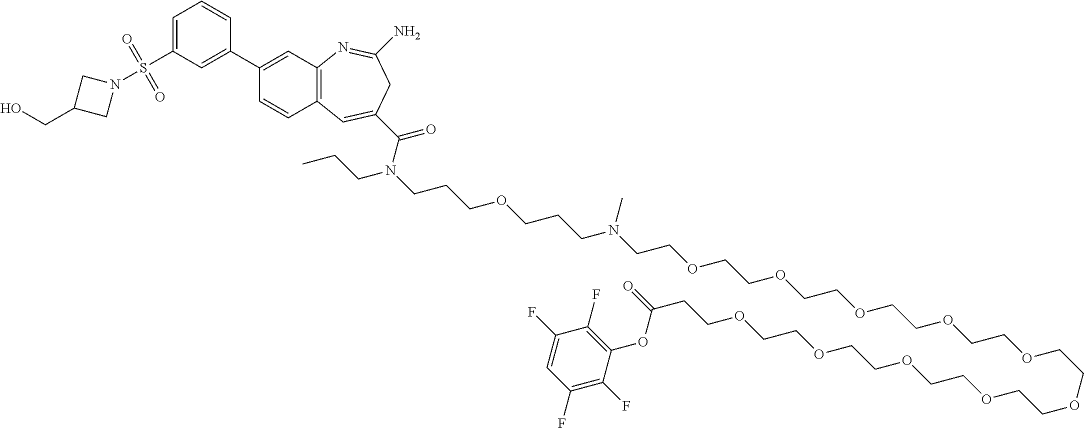 [SMILES: CCCN(CCCOCCCN(C)CCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CO)C4)c3)cc2N=C(N)C1] | 1258.4 |
| BzL-38 | 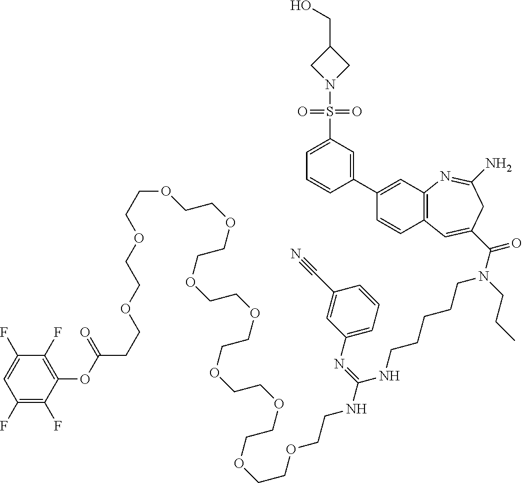 [SMILES: CCCN(CCCCCN/C(=N\c1cccc(C#N)c1)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CO)C4)c3)cc2N=C(N)C1] | 1357.5 |
| BzL-39 | 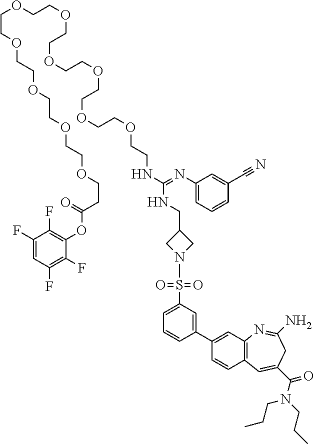 [SMILES: CCCN(CCC)C(=O)C1=Cc2ccc(-c3cccc(S(=O)(=O)N4CC(CN/C(=N\c5cccc(C#N)c5)NCCOCCOCCOCCOCCOCCOCCOCCOCCOCCOCCC(=O)Oc5c(F)c(F)cc(F)c5F)C4)c3)cc2N=C(N)C1] | 1313.5 |
| BzL-40 | 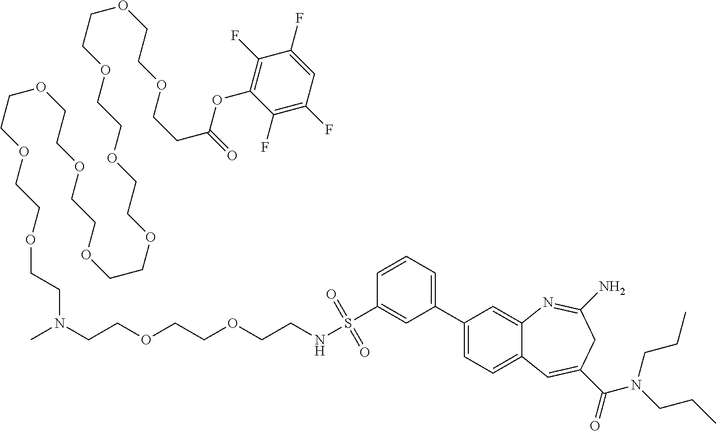 [SMILES: CCCN(CCC)C(=O)C1=Cc2ccc(-c3cccc(S(=O)(=O)NCCOCCOCCN(C)CCOCCOCCOCCOCCOCCOCCOCCOCCOCCOCCC(=O)Oc4c(F)c(F)cc(F)c4F)c3)cc2N=C(N)C1] | 1246.4 |
| BzL-41 | 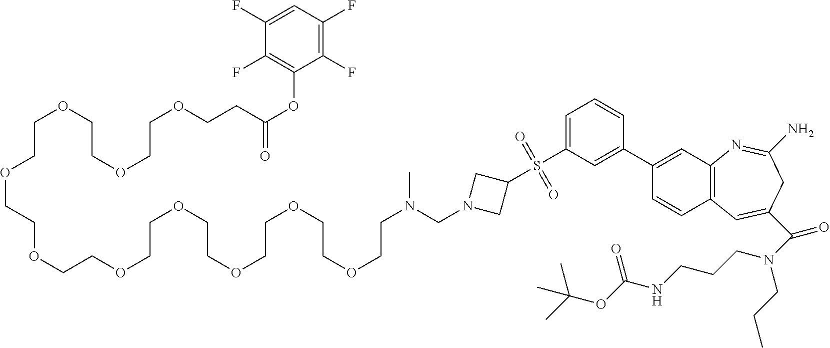 [SMILES: CCCN(CCCNC(=O)OC(C)(C)C)C(=O)C1=Cc2ccc(-c3cccc(S(=O)(=O)C4CN(CN(C)CCOCCOCCOCCOCCOCCOCCOCCOCCOCCOCCC(=O)Oc5c(F)c(F)cc(F)c5F)C4)c3)cc2N=C(N)C1] | 1299.5 |
| BzL-42 | 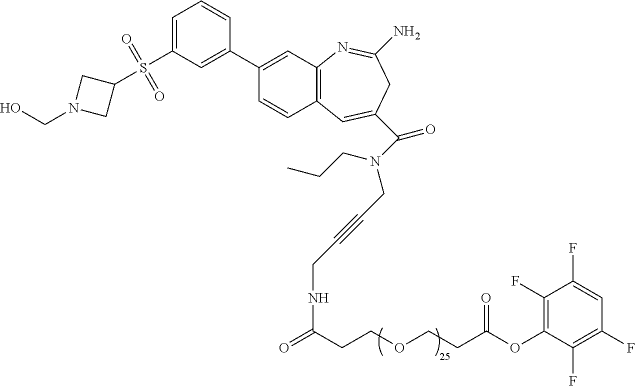 | 1885.1 |
| BzL-43 | 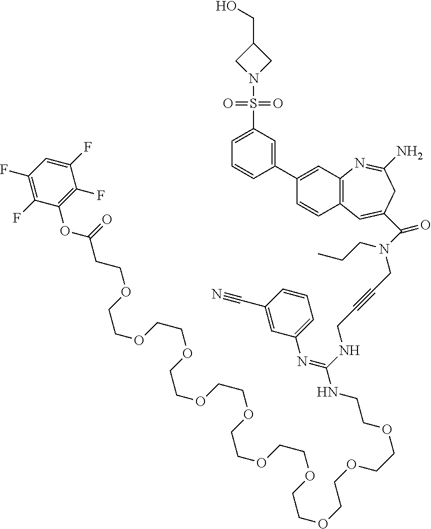 [SMILES: CCCN(CC#CCN/C(=N\c1cccc(C#N)c1)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CO)C4)c3)cc2N=C(N)C1] | 1339.5 |
| BzL-44 | 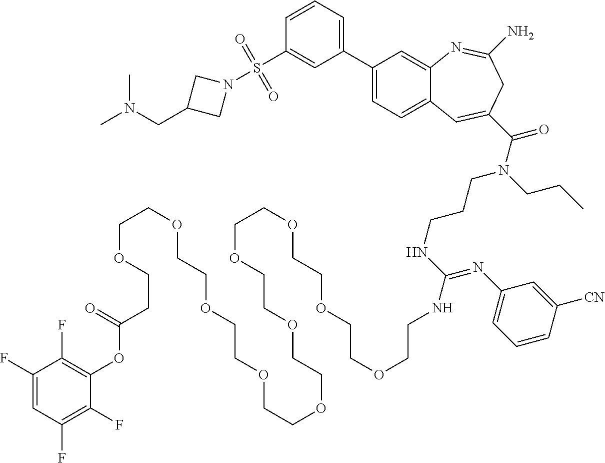 [SMILES: CCCN(CCCN/C(=N/c1cccc(C#N)c1)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CN(C)C)C4)c3)cc2N=C(N)C1] | 1356.5 |
| BzL-45 | 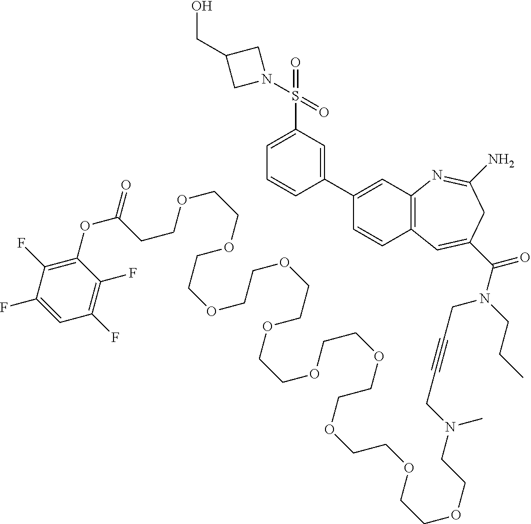 [SMILES: CCCN(CC#CCN(C)CCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CO)C4)c3)cc2N=C(N)C1] | 1210.3 |
| BzL-46 | 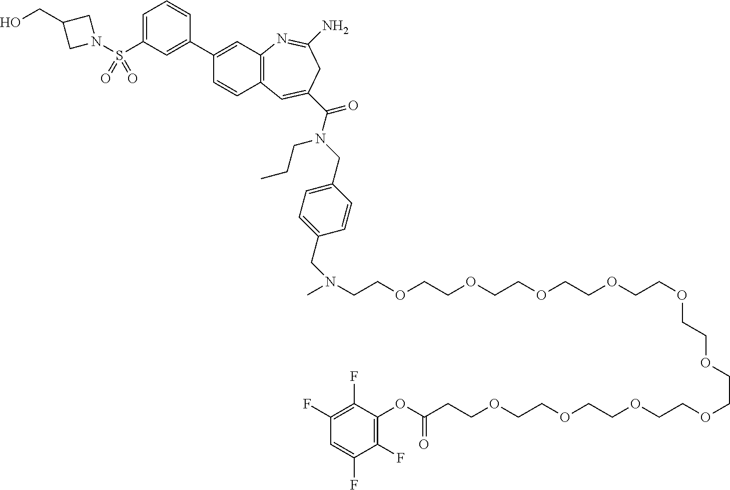 [SMILES: CCCN(Cc1ccc(CN(C)CCOCCOCCOCCOCCOCCOCCOCCOCCOCCOCCC(=O)Oc2c(F)c(F)cc(F)c2F)cc1)C(=O)C1=Cc2ccc(-c3cccc(S(=O)(=O)N4CC(CO)C4)c3)cc2N=C(N)C1] | 1262.4 |
| BzL-47 | 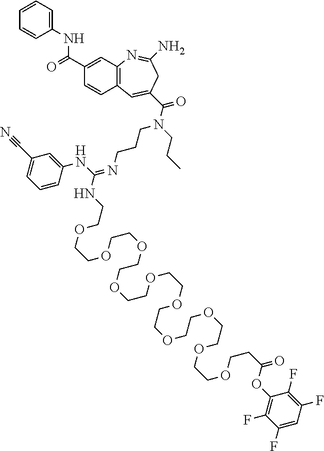 [SMILES: CCCN(CCC/N=C(/NCCOCCOCCOCCOCCOCCOCCOCCOCCOCCOCCC(=O)Oc1c(F)c(F)cc(F)c1F)Nc1cccc(C#N)c1)C(=O)C1=Cc2ccc(C(=O)Nc3ccccc3)cc2N=C(N)C1] | 1223.3 |
| BzL-48 | 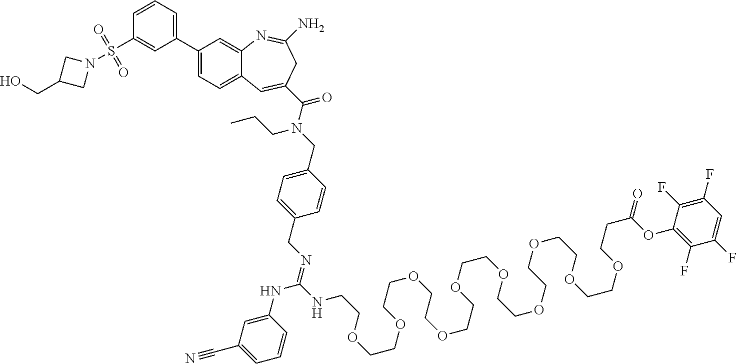 [SMILES: CCCN(Cc1ccc(C/N=C(/NCCOCCOCCOCCOCCOCCOCCOCCOCCOCCOCCC(=O)Oc2c(F)c(F)cc(F)c2F)Nc2cccc(C#N)c2)cc1)C(=O)C1=Cc2ccc(-c3cccc(S(=O)(=O)N4CC(CO)C4)c3)cc2N=C(N)C1] | 1391.5 |

TABLE 2c
Aminobenzazepine-linker Formula II compounds (BzL) and intermediates

| BzL | | |
|---|---|---|
| No. | Structure | MW |
| BzL-49 | 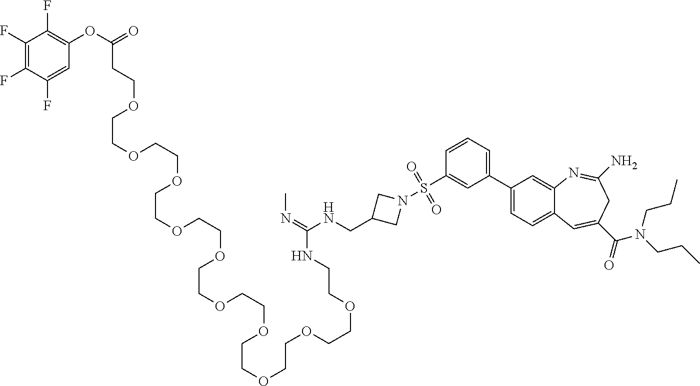 [SMILES: CCCN(CCC)C(=O)C1=Cc2ccc(-c3cccc(S(=O)(=O)N4CC(CN/C(=N\C)NCCOCCOCCOCCOCCOCCOCCOCCOCCOCCOCCC(=O)Oc5cc(F)c(F)c(F)c5F)C4)c3)cc2N=C(N)C1] | 1226.4 |
| BzL-50 | 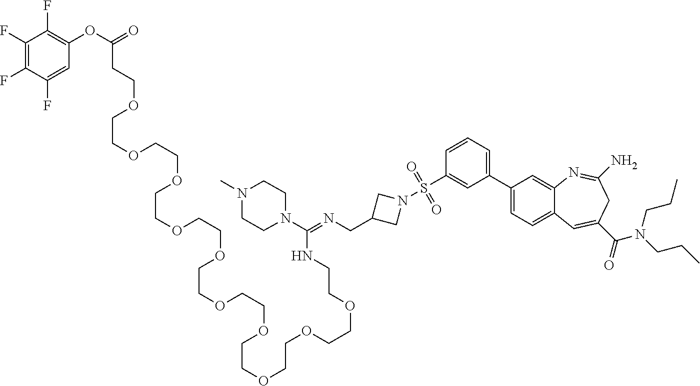 [SMILES: CCCN(CCC)C(=O)C1=Cc2ccc(-c3cccc(S(=O)(=O)N4CC(C/N=C(\NCCOCCOCCOCCOCCOCCOCCOCCOCCOCCOCCC(=O)Oc5cc(F)c(F)c(F)c5F)N5CCN(C)CC5)C4)c3)cc2N=C(N)C1] | 1295.5 |
| BzL-51 | 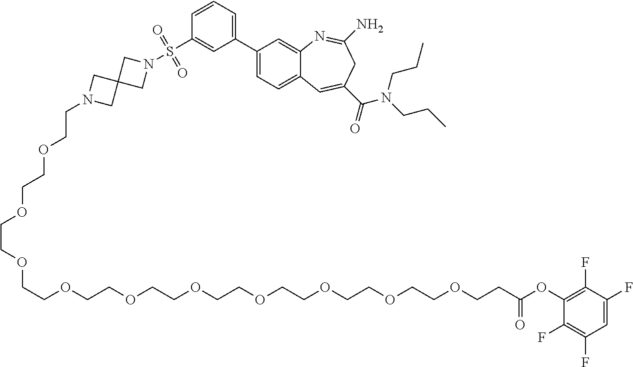 [SMILES: CCCN(CCC)C(=O)C1=Cc2ccc(-c3cccc(S(=O)(=O)N4CC5(CN(CCOCCOCCOCCOCCOCCOCCOCCOCCOCCOCCC(=O)Oc6c(F)c(F)cc(F)c6F)C5)C4)c3)cc2N=C(N)C1] | 1182.3 |
| BzL-52 | 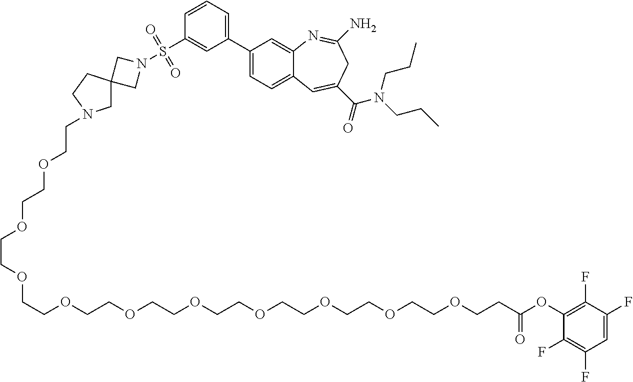 [SMILES: CCCN(CCC)C(=O)C1=Cc2ccc(-c3cccc(S(=O)(=O)N4CC5(CCN(CCOCCOCCOCCOCCOCCOCCOCCOCCOCCOCCC(=O)Oc6c(F)c(F)cc(F)c6F)C5)C4)c3)cc2N=C(N)C1] | 1196.4 |
| BzL-53 | 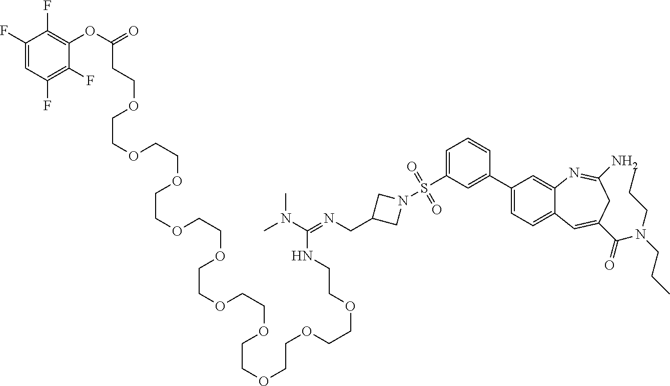 [SMILES: CCCN(CCC)C(=O)C1=Cc2ccc(-c3cccc(S(=O)(=O)N4CC(C/N=C(\NCCOCCOCCOCCOCCOCCOCCOCCOCCOCCOCCC(=O)Oc5c(F)c(F)cc(F)c5F)N(C)C)C4)c3)cc2N=C(N)C1] | 1240.4 |
| BzL-54 | 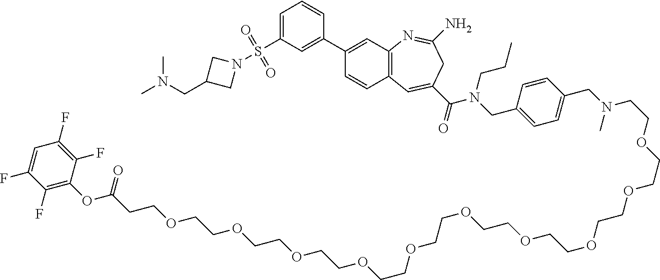 [SMILES: CCCN(Cc1ccc(CN(C)CCOCCOCCOCCOCCOCCOCCOCCOCCOCCOCCC(=O)Oc2c(F)c(F)cc(F)c2F)cc1)C(=O)C1=Cc2ccc(-c3cccc(S(=O)(=O)N4CC(CN(C)C)C4)c3)cc2N=C(N)C1] | 1289.5 |
| BzL-55 | 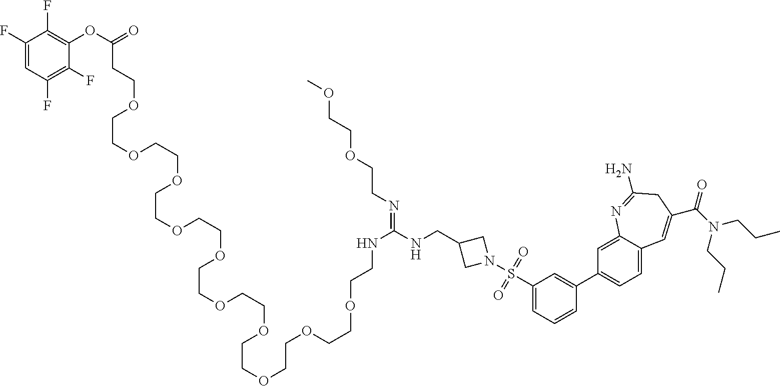 [SMILES: CCCN(CCC)C(=O)C1=Cc2ccc(-c3cccc(S(=O)(=O)N4CC(CN/C(=N/CCOCCOC)NCCOCCOCCOCCOCCOCCOCCOCCOCCOCCOCCC(=O)Oc5c(F)c(F)cc(F)c5F)C4)c3)cc2N=C(N)C1] | 1314.5 |
| BzL-56 | 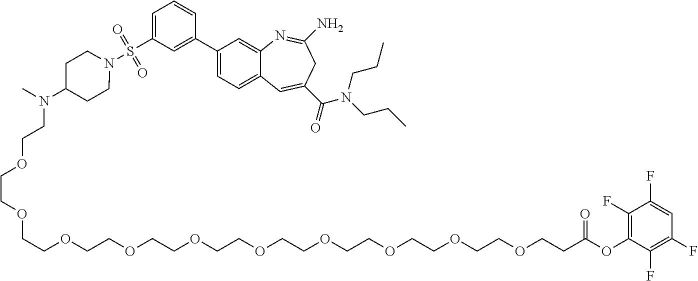 [SMILES: CCCN(CCC)C(=O)C1=Cc2ccc(-c3cccc(S(=O)(=O)N4CCC(N(C)CCOCCOCCOCCOCCOCCOCCOCCOCCOCCOCCC(=O)Oc5c(F)c(F)cc(F)c5F)CC4)c3)cc2N=C(N)C1] | 1198.4 |
| BzL-57 | 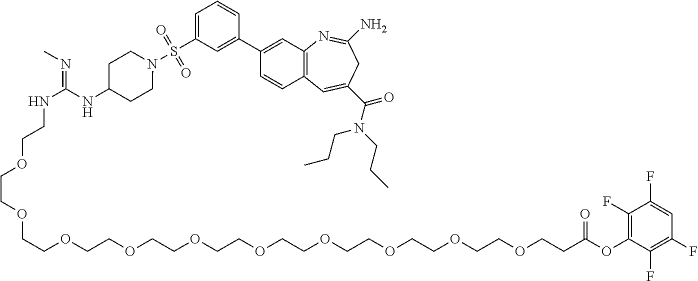 [SMILES: CCCN(CCC)C(=O)C1=Cc2ccc(-c3cccc(S(=O)(=O)N4CCC(N/C(=N/C)NCCOCCOCCOCCOCCOCCOCCOCCOCCOCCOCCC(=O)Oc5c(F)c(F)cc(F)c5F)CC4)c3)cc2N=C(N)C1] | 1240.4 |
| BzL-58 | 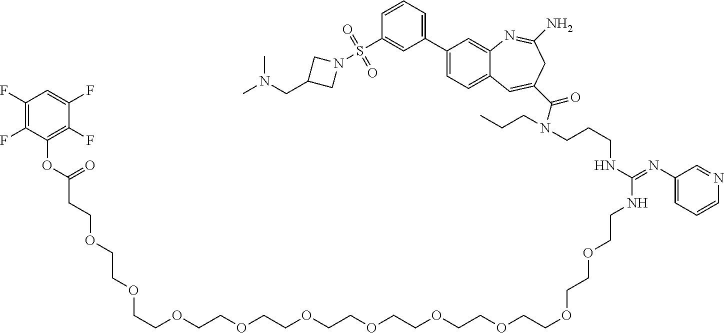 [SMILES: CCCN(CCCN/C(=N/c1cccnc1)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CN(C)C)C4)c3)cc2N=C(N)C1] | 1332.5 |
| BzL-59 | 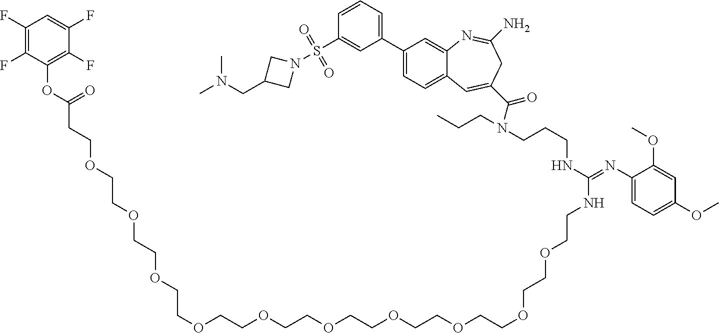 [SMILES: CCCN(CCCN/C(=N/c1ccc(OC)cc1OC)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CN(C)C)C4)c3)cc2N=C(N)C1] | 1391.6 |
| BzL-60 | 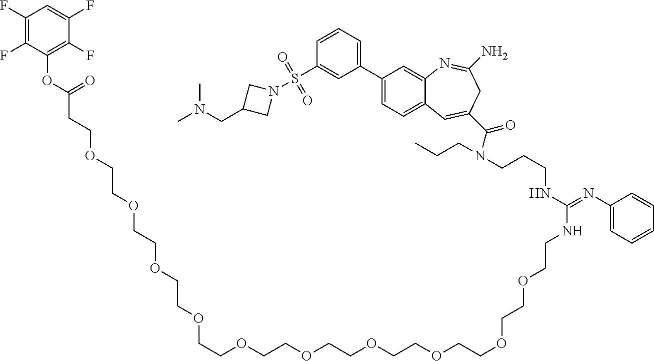 [SMILES: CCCN(CCCN/C(=N/c1ccccc1)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CN(C)C)C4)c3)cc2N=C(N)C1] | 1331.5 |
| BzL-61 | 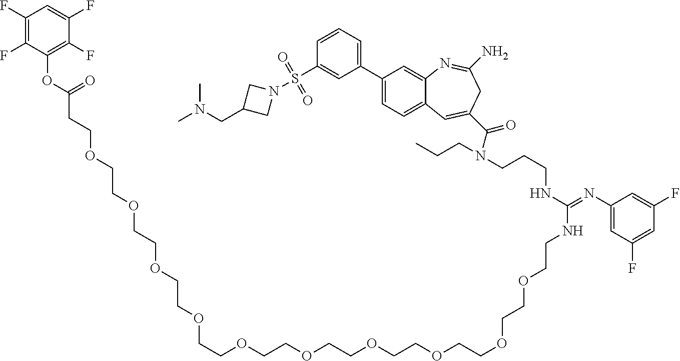 [SMILES: CCCN(CCCN/C(=N/c1cc(F)cc(F)c1)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CN(C)C)C4)c3)cc2N=C(N)C1] | 1367.5 |
| BzL-62 | 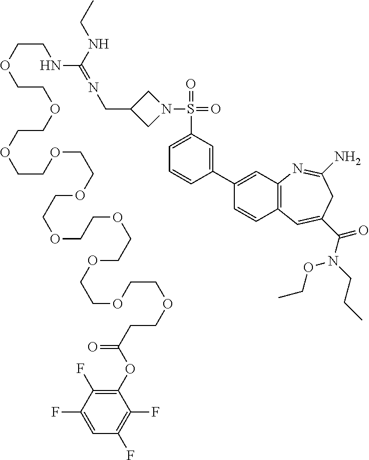 [SMILES: CCCN(OCC)C(=O)C1=Cc2ccc(-c3cccc(S(=O)(=O)N4CC(CN=C(NCC)NCCOCCOCCOCCOCCOCCOCCOCCOCCOCCOCCC(=O)Oc5c(F)c(F)cc(F)c5F)C4)c3)cc2N=C(N)C1] | 1242.4 |
| BzL-63 | 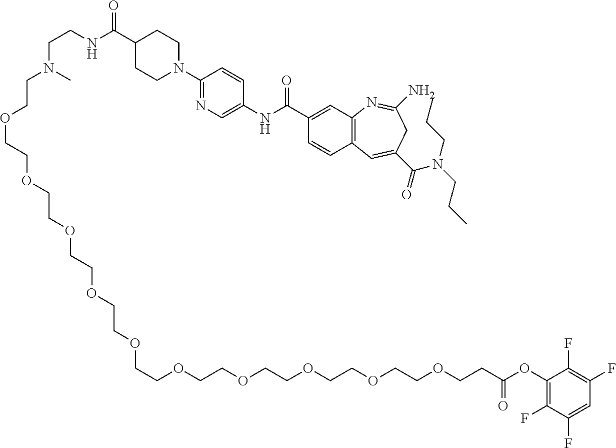 [SMILES: CCCN(CCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1] | 1249.4 |
| BzL-64 | 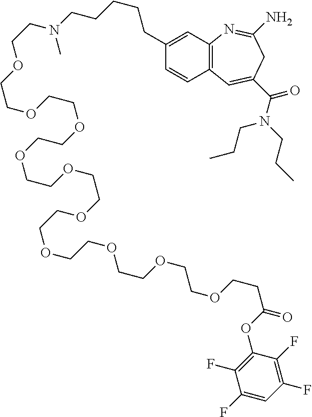 [SMILES: CCCN(CCC)C(=O)C1=Cc2ccc(CCCCCN(C)CCOCCOCCOCCOCCOCCOCCOCCOCCOCCOCCC(=O)Oc3c(F)c(F)cc(F)c3F)cc2N=C(N)C1] | 1045.2 |
| BzL-65 | 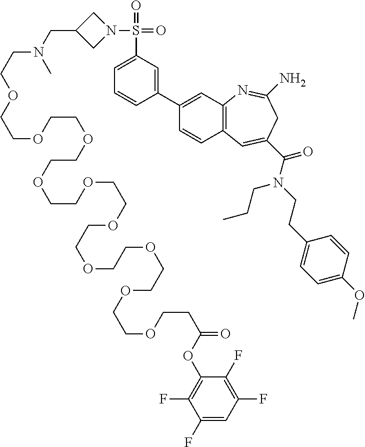 [SMILES: CCCN(CCc1ccc(OC)cc1)C(=O)C1=Cc2ccc(-c3cccc(S(=O)(=O)N4CC(CN(C)CCOCCOCCOCCOCCOCCOCCOCCOCCOCCOCCC(=O)Oc5c(F)c(F)cc(F)c5F)C4)c3)cc2N=C(N)C1] | 1276.4 |
| BzL-66 | 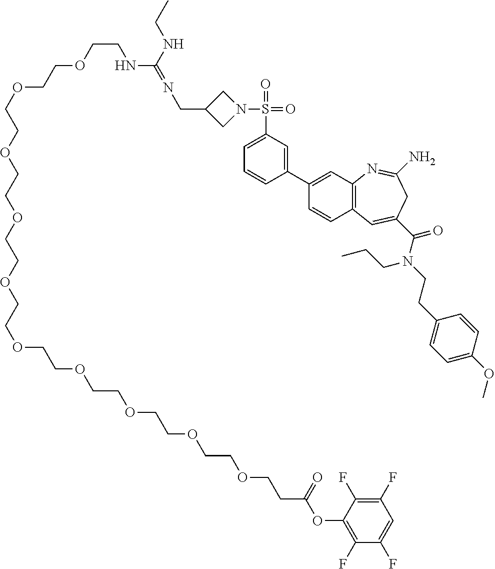 [SMILES: CCCN(CCc1ccc(OC)cc1)C(=O)C1=Cc2ccc(-c3cccc(S(=O)(=O)N4CC(CN=C(NCC)NCCOCCOCCOCCOCCOCCOCCOCCOCCOCCOCCC(=O)Oc5c(F)c(F)cc(F)c5F)C4)c3)cc2N=C(N)C1] | 1332.5 |
| BzL-67 | 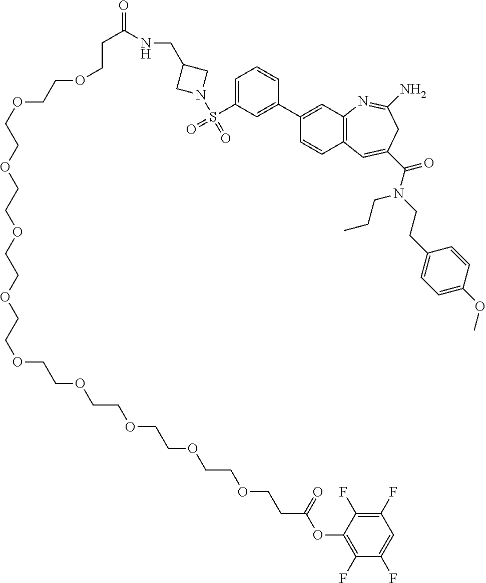 [SMILES: CCCN(CCc1ccc(OC)cc1)C(=O)C1=Cc2ccc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1290.4 |
| BzL-68 | 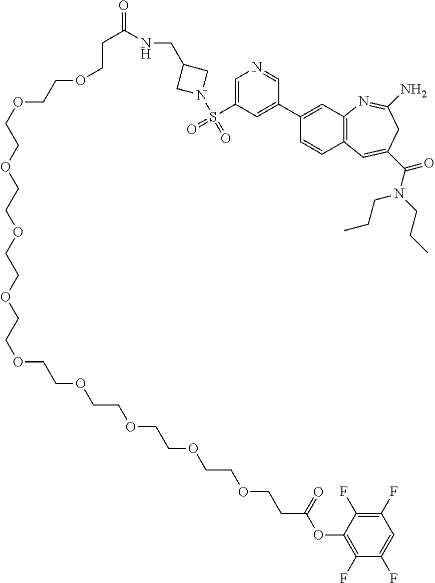 [SMILES: CCCN(CCC)C(=O)C1=Cc2ccc(-c3cncc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1199.3 |
| BzL-69 | 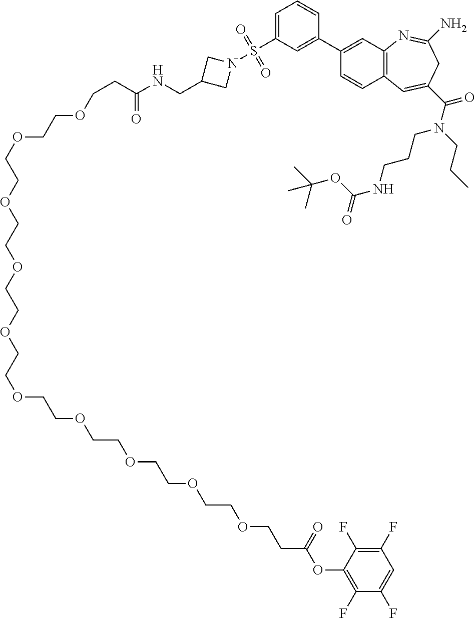 [SMILES: CCCN(CCCNC(=O)OC(C)(C)C)C(=O)C1=Cc2ccc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1313.5 |
| BzL-70 | 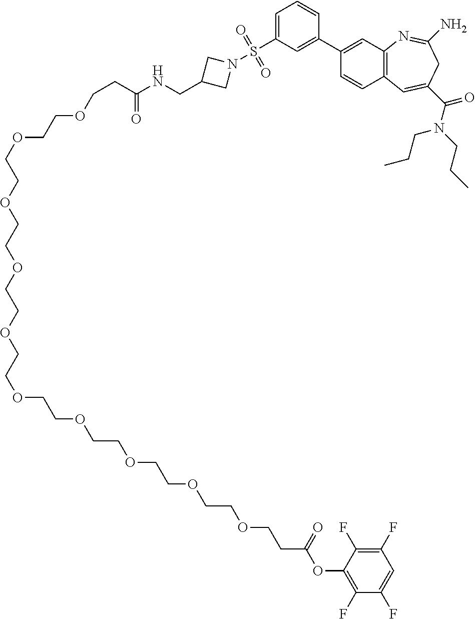 [SMILES: CCCN(CCC)C(=O)C1=Cc2ccc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1198.3 |
| BzL-71 | 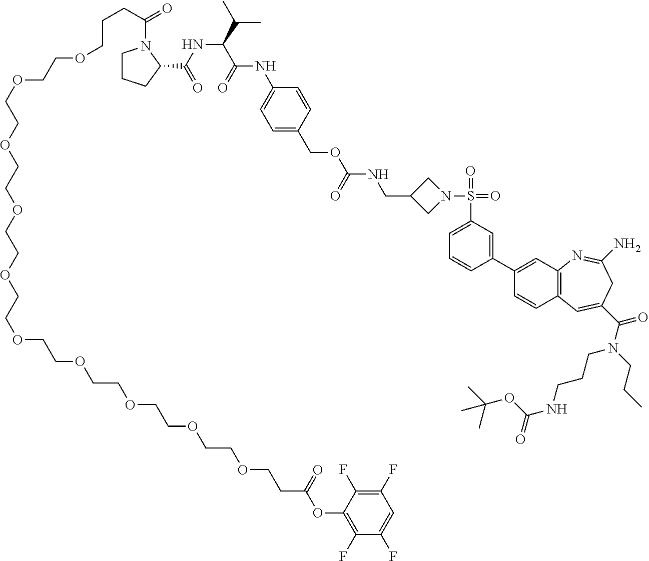 [SMILES: CCCN(CCCNC(=O)OC(C)(C)C)C(=O)C1=Cc2ccc(-c3cccc(S(=O)(=O)N4CC(CNC(=O)OCc5ccc(NC(=O)[C@@H](NC(=O)[C@@H]6CCCN6C(=O)CCCOCCOCCOCCOCCOCCOCCOCCOCCOCCOCCC(=O)Oc6c(F)c(F)cc(F)c6F)C(C)C)cc5)C4)c3)cc2N=C(N)C1] | 1658.9 |
| BzL-72 | 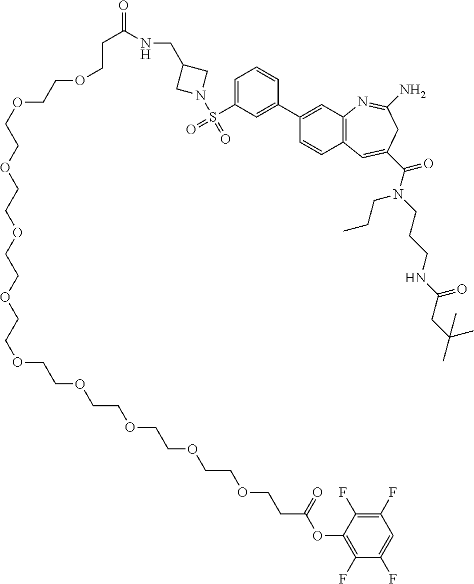 [SMILES: CCCN(CCCNC(=O)CC(C)(C)C)C(=O)C1=Cc2ccc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1311.5 |
| BzL-73 | 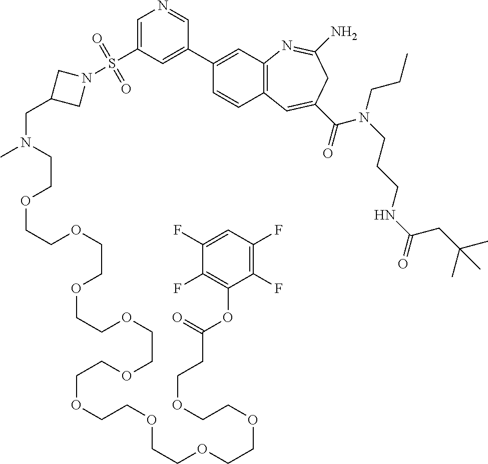 [SMILES: CCCN(CCCNC(=O)CC(C)(C)C)C(=O)C1=Cc2ccc(-c3cncc(S(=O)(=O)N4CC(CN(C)CCOCCOCCOCCOCCOCCOCCOCCOCCOCCOCCC(=O)Oc5c(F)c(F)cc(F)c5F)C4)c3)cc2N=C(N)C1] | 1298.5 |
| BzL-74 | 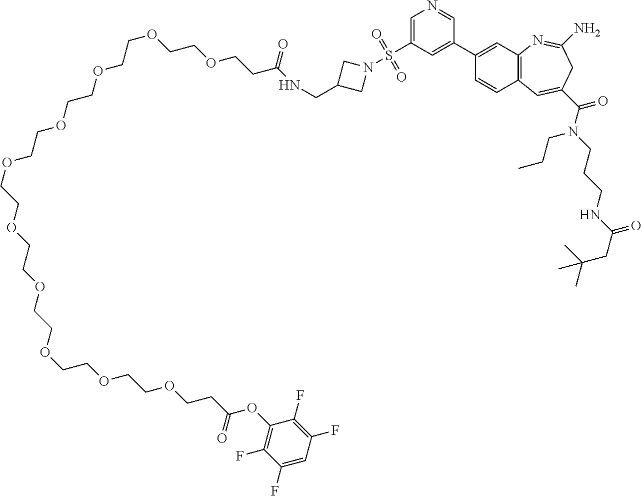 [SMILES: CCCN(CCCNC(=O)CC(C)(C)C)C(=O)C1=Cc2ccc(-c3cncc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1312.5 |
| BzL-75 | 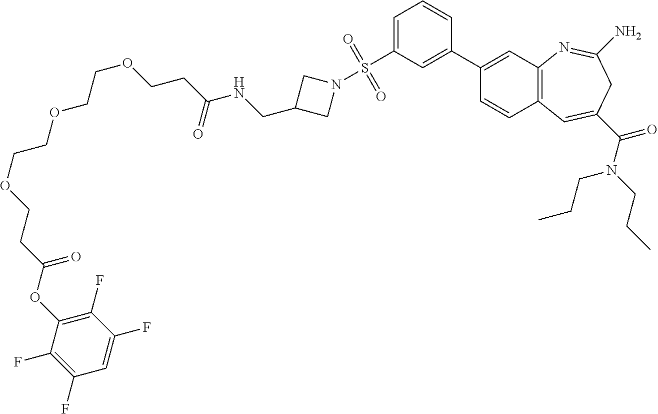 [SMILES: CCCN(CCC)C(=O)C1=Cc2ccc(-c3cccc(S(=O)(=O)N4CC(CNC(=O)CCOCCOCCOCCC(=O)Oc5c(F)c(F)cc(F)c5F)C4)c3)cc2N=C(N)C1] | 890.0 |
| BzL-76 | 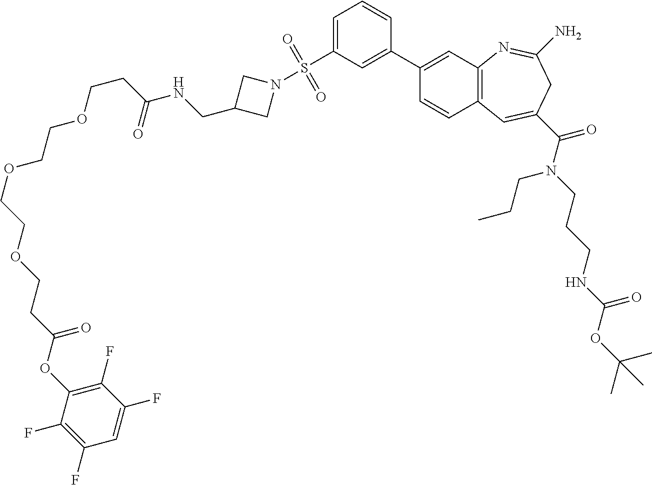 [SMILES: CCCN(CCCNC(=O)OC(C)(C)C)C(=O)C1=Cc2ccc(-c3cccc(S(=O)(=O)N4CC(CNC(=O)CCOCCOCCOCCC(=O)Oc5c(F)c(F)cc(F)c5F)C4)c3)cc2N=C(N)C1] | 1005.1 |
| BzL-77 | 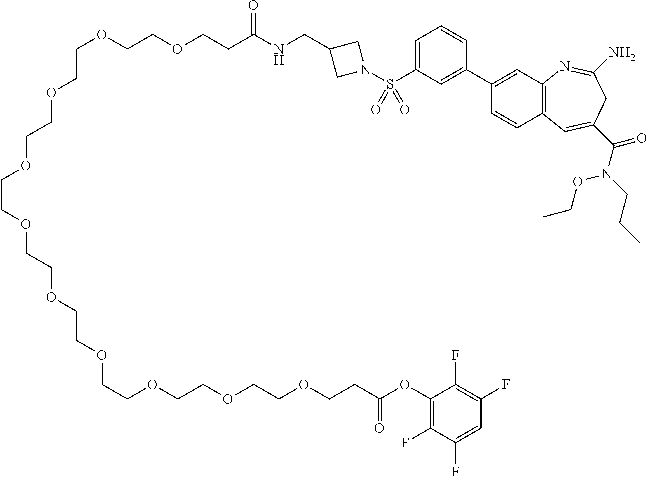 [SMILES: CCCN(OCC)C(=O)C1=Cc2ccc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1200.3 |
| BzL-78 | 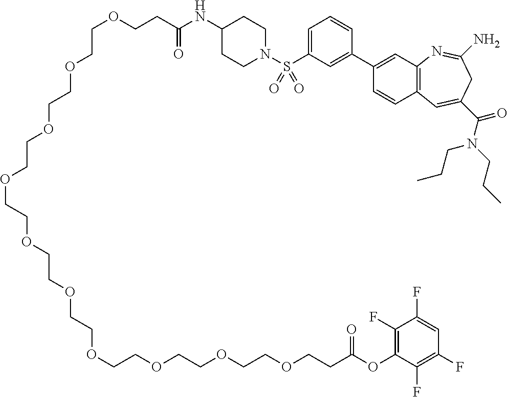 [SMILES: CCCN(CCC)C(=O)C1=Cc2ccc(-c3cccc(S(=O)(=O)N4CCC(NC(=O)CCOCCOCCOCCOCCOCCOCCOCCOCCOCCOCCC(=O)Oc5c(F)c(F)cc(F)c5F)CC4)c3)cc2N=C(N)C1] | 1212.4 |
| BzL-79 | 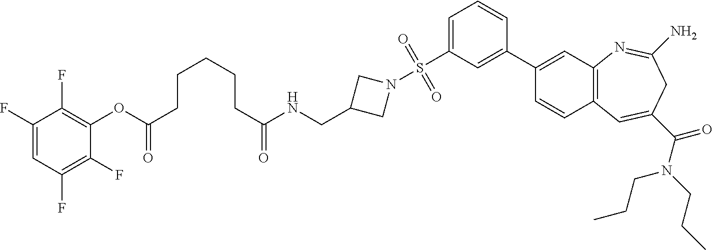 [SMILES: CCCN(CCC)C(=O)C1=Cc2ccc(-c3cccc(S(=O)(=O)N4CC(CNC(=O)CCCCCC(=O)Oc5c(F)c(F)cc(F)c5F)C4)c3)cc2N=C(N)C1] | 799.9 |
| BzL-80 | 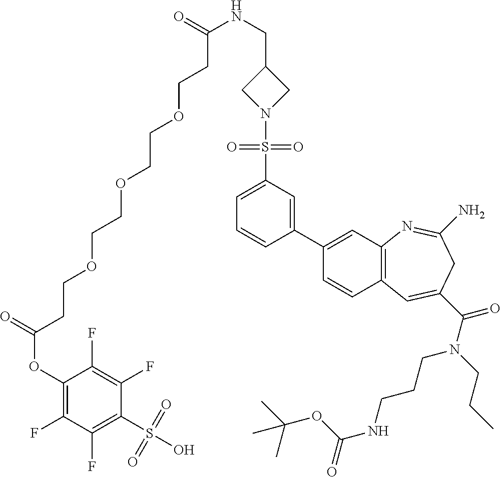 [SMILES: CCCN(CCCNC(=O)OC(C)(C)C)C(=O)C1=Cc2ccc(-c3cccc(S(=O)(=O)N4CC(CNC(=O)CCOCCOCCOCCC(=O)Oc5c(F)c(F)c(S(=O)(=O)O)c(F)c5F)C4)c3)cc2N=C(N)C1] | 1085.1 |
| BzL-81 | 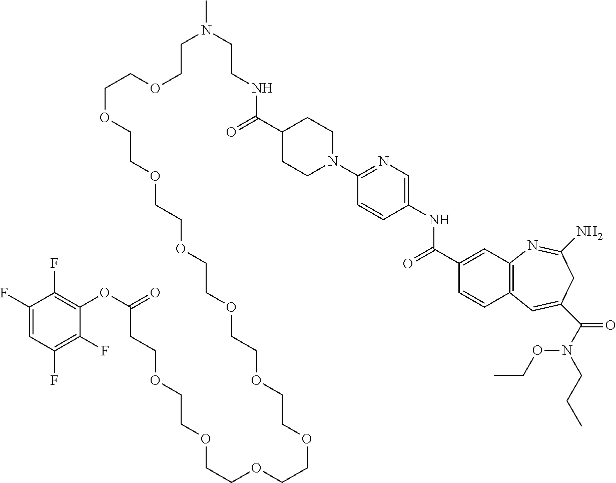 [SMILES: CCCN(OCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1] | 1251.4 |
| BzL-82 | 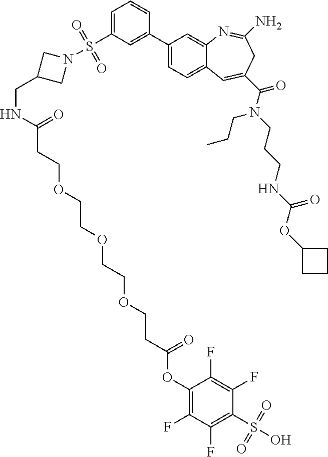 [SMILES: CCCN(CCCNC(=O)OC1CCC1)C(=O)C1=Cc2ccc(-c3cccc(S(=O)(=O)N4CC(CNC(=O)CCOCCOCCOCCC(=O)Oc5c(F)c(F)c(S(=O)(=O)O)c(F)c5F)C4)c3)cc2N=C(N)C1] | 1083.1 |
| BzL-83 | 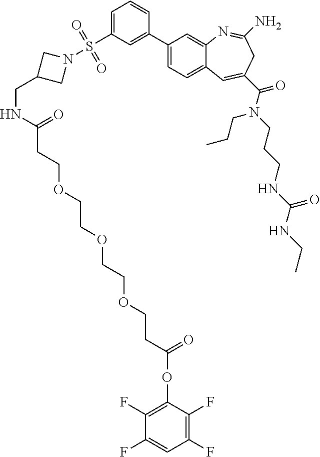 [SMILES: CCCN(CCCNC(=O)NCC)C(=O)C1=Cc2ccc(-c3cccc(S(=O)(=O)N4CC(CNC(=O)CCOCCOCCOCCC(=O)Oc5c(F)c(F)cc(F)c5F)C4)c3)cc2N=C(N)C1] | 976.1 |
| BzL-84 | 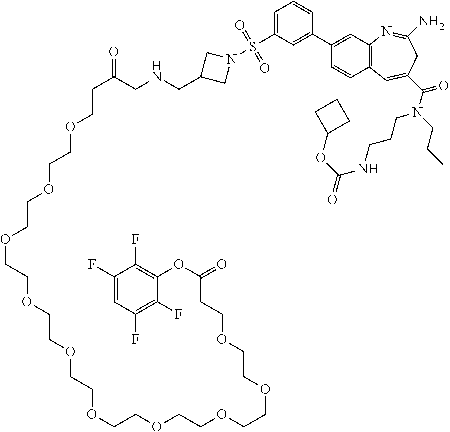 [SMILES: CCCN(CCCNC(=O)OC1CCC1)C(=O)C1=Cc2ccc(-c3cccc(S(=O)(=O)N4CC(CNCC(=O)CCOCCOCCOCCOCCOCCOCCOCCOCCOCCOCCC(=O)Oc5c(F)c(F)cc(F)c5F)C4)c3)cc2N=C(N)C1] | 1325.5 |
| BzL-85 | 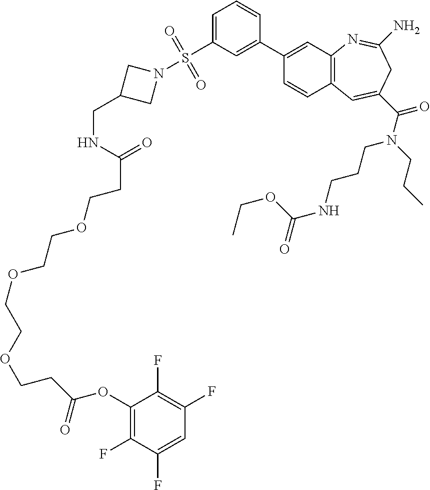 [SMILES: CCCN(CCCNC(=O)OCC)C(=O)C1=Cc2ccc(-c3cccc(S(=O)(=O)N4CC(CNC(=O)CCOCCOCCOCCC(=O)Oc5c(F)c(F)cc(F)c5F)C4)c3)cc2N=C(N)C1] | 977.0 |
| BzL-86 | 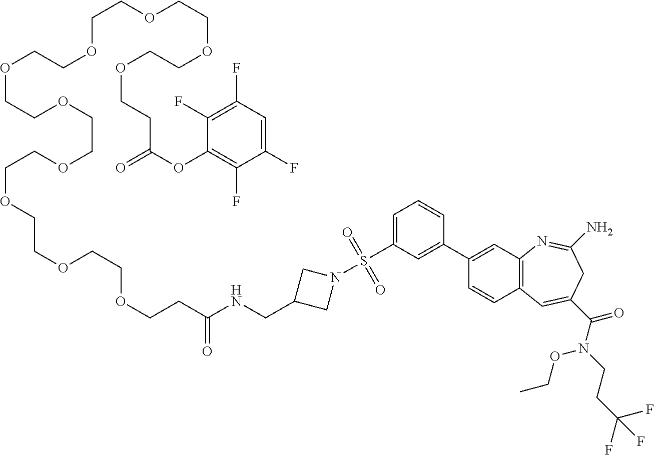 [SMILES: CCON(CCC(F)(F)F)C(=O)C1=Cc2ccc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1254.3 |
| BzL-87 | 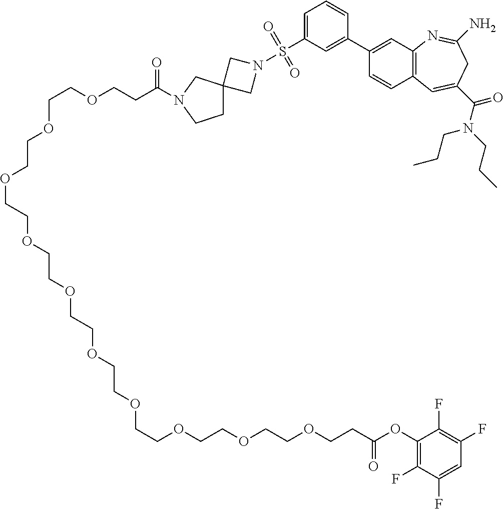 [SMILES: CCCN(CCC)C(=O)C1=Cc2ccc(-c3cccc(S(=O)(=O)N4CC5(CCN(C(=O)CCOCCOCCOCCOCCOCCOCCOCCOCCOCCOCCC(=O)Oc6c(F)c(F)cc(F)c6F)C5)C4)c3)cc2N=C(N)C1] | 1224.4 |

### Immunoconjujgates

Exemplary embodiments of immunoconjugates comprise an antibody covalently attached to a divalent linker which is covalently attached to one or more aminobenzazepine moieties, and having Formula I:

Ab-[L-Bza]ₚ
or a pharmaceutically acceptable salt thereof,
wherein:
Ab is the antibody;
p is an integer from 1 to 8;
Bza is the aminobenzazepine moiety having the formula:

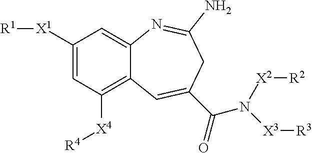

R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-NR 5-C(═NR^{5a})NR⁵—*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—C(═O)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₅ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)—(C₂-C₅ heteroaryl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring; X¹, X², X³, and X⁴ are independently selected from the group consisting of a bond, C(═O), C(═O)N(R⁵), 0, N(R⁵), S, S(O)₂, and S(O)₂N(R⁵);
R⁵ is selected from the group consisting of H, C₆-C₂₀ aryl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
—C(═O)-(PEG)-;
—C(═O)-(PEG)-C(═O)—;
—C(═O)-(PEG)-O—;
—C(═O)-(PEG)-C(═O)-(PEP)-;
—C(═O)-(PEG)-C(═O)N(R⁵)—(C₁-C₁₂ alkyldiyl)-;
—C(═O)-(PEG)-C(═O)N(R⁵)—(C₁-C₁₂ alkyldiyl)-N(R⁵)C(═O)—(C₂-C₅ monoheterocyclyldiyl)-;
—C(═O)-(PEG)-C(═O)N(R⁵)—(C₁-C₁₂ alkyldiyl)-(MCgluc)-;
—C(═O)-(PEG)-C(═O)-(MCgluc)-;
—C(═O)-(PEG)-C(═O)-(PEP)-N(R⁵)—(C₁-C₁₂ alkyldiyl)-;
—C(═O)-(PEG)-C(═O)-(PEP)-N(R⁵)—(C₁-C₁₂ alkyldiyl)-N(R⁵)C(═O)—(C₂-C₅ monoheterocyclyldiyl)-;
—C(═O)-(PEG)-N(R⁵)—;
—C(═O)-(PEG)-N(R⁵)—(PEG)-C(═O)-(PEP)-;
—C(═O)-(PEG)-N⁺(R⁵)₂-(PEG)-C(═O)-(PEP)-;
—C(═O)-(PEG)-C(═O)—N(R⁵)CH(AA₁)C(═O)-(PEG)-C(═O)-(PEP)-;
—C(═O)-(PEG)-C(═O)—N(R⁵)CH(AA₁)C(═O)—N(R⁵)—(C₁-C₁₂ alkyldiyl)-;
—C(═O)-(PEG)-SS—(C₁-C₁₂ alkyldiyl)-OC(═O)—;
—C(═O)-(PEG)-SS—(C₁-C₁₂ alkyldiyl)-C(═O)—;
—C(═O)—(C₁-C₁₂ alkyldiyl)-C(═O)-(PEP)-;
—C(═O)—(C₁-C₁₂ alkyldiyl)-C(═O)-(PEP)-N(R⁵)—(C₁-C₁₂ alkyldiyl)-;
—C(═O)—(C₁-C₁₂ alkyldiyl)-C(═O)-(PEP)-N(R⁵)—(C₁-C₁₂ alkyldiyl)-N(R⁵)—C(═O);
—C(═O)—(C₁-C₁₂ alkyldiyl)-C(═O)-(PEP)-N(R⁵)—(C₁-C₁₂ alkyldiyl)-N(R⁵)C(═O)—(C₂-C₅ monoheterocyclyldiyl)-;
—C(═O)—CH₂CH₂OCH₂CH₂—(C₁-C₂₀ heteroaryldiyl)-CH₂O-(PEG)-C(═O)-(MCgluc)-;
—C(═O)—CH₂CH₂OCH₂CH₂—(C₁-C₂₀ heteroaryldiyl)-CH₂O-(PEG)-C(═O)-(MCgluc)-N(R⁵)—(C₁-C₁₂ alkyldiyl)-N(R⁵)C(═O)—(C₂-C₅ monoheterocyclyldiyl)-; and
(succinimidyl)-(CH₂)ₘ—C(═O)-(PEP)-N(R⁵)—(C₁-C₁₂ alkyldiyl)-N(R⁵)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
PEP has the formula:

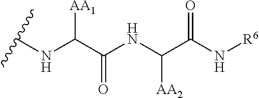

where AA₁ and AA₂ are independently selected from an amino acid side chain, or AA₁ or AA₂ and an adjacent nitrogen atom form a 5-membered ring proline amino acid, and the wavy line indicates a point of attachment;
R⁶ is selected from the group consisting of C₆-C₂₀ aryldiyl and C₁-C₂₀ heteroaryldiyl, substituted with —CH₂O—C(═O)— and optionally with:

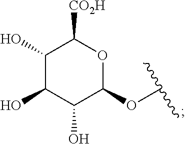 [SMILES: *O[C@@H]1O[C@H](C(=O)O)[C@@H](O)[C@H](O)[C@H]1O]

and MCgluc is selected from the groups:

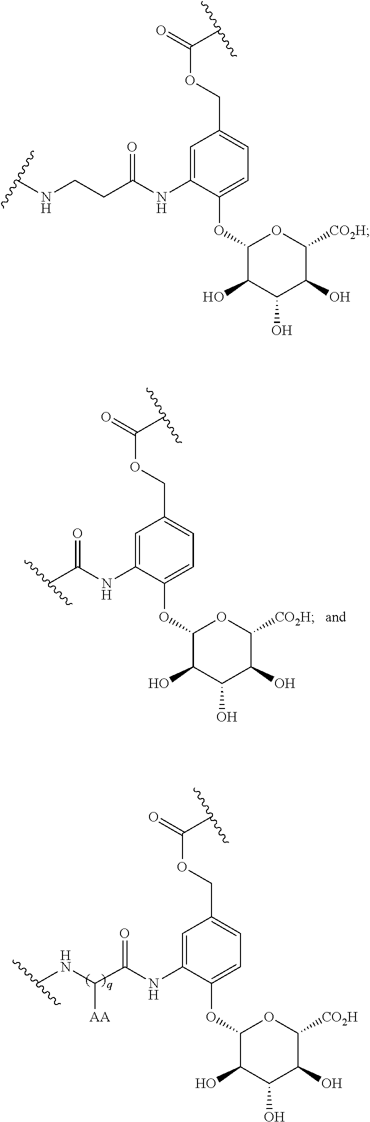

where q is 1 to 8, and AA is an amino acid side chain; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)˜H, —OP(O)(OH)₂, —S(O)₂N(CH₃)₂, —SCH₃, —S(O)₂CH₃, and —S(O)₃H.

An exemplary embodiment of the immunoconjugate of Formula I includes wherein the antibody is an antibody construct that has an antigen binding domain that binds PD-L1

An exemplary embodiment of the immunoconjugate of Formula I includes wherein the antibody is selected from the group consisting of atezolizumab, durvalumab, and avelumab, or a biosimilar or a biobetter thereof.

An exemplary embodiment of the immunoconjugate of Formula I includes wherein the antibody is an antibody construct that has an antigen binding domain that binds HER2.

An exemplary embodiment of the immunoconjugate of Formula I includes wherein the antibody is selected from the group consisting of trastuzumab and pertuzumab, or a biosimilar or a biobetter thereof.

An exemplary embodiment of the immunoconjugate of Formula I includes wherein the antibody is an antibody construct that has an antigen binding domain that binds CEA.

An exemplary embodiment of the immunoconjugate of Formula I includes wherein the antibody is labetuzumab, or a biosimilar or a biobetter thereof.

An exemplary embodiment of the immunoconjugate of Formula I includes wherein PEP is selected from the groups:

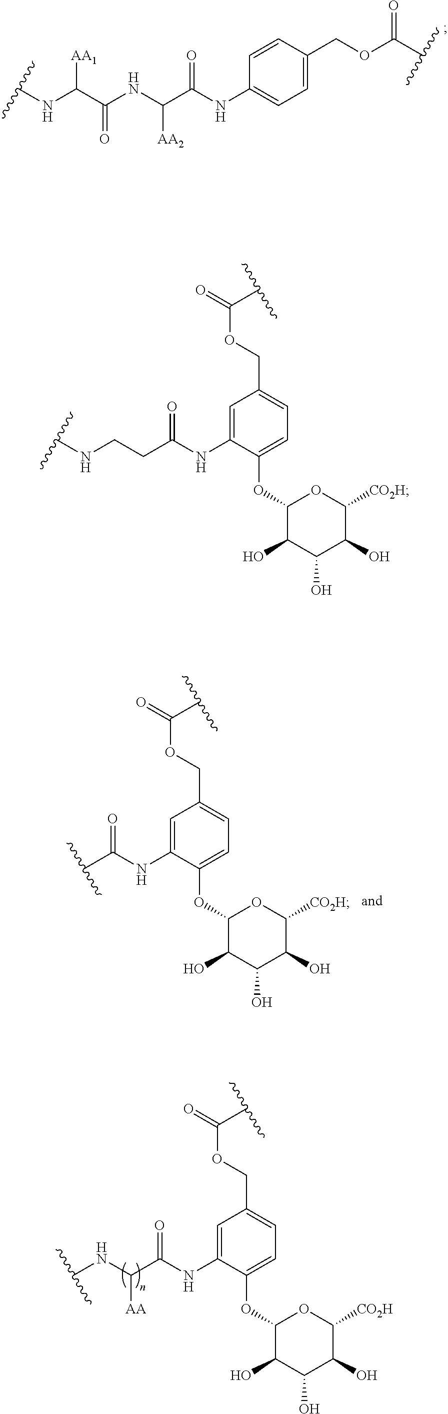

where n is 1 or more, and AA is an amino acid side chain.

An exemplary embodiment of the immunoconjugate of Formula I includes wherein AA₁ and AA₂ are independently selected from a side chain of a naturally-occurring amino acid.

An exemplary embodiment of the immunoconjugate of Formula I includes wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CH₂CH(CH₃)₂, —CH₂SO₃H, and —CH₂CH₂CH₂NHC(O)NH₂.

An exemplary embodiment of the immunoconjugate of Formula I includes wherein AA₁ is —CH(CH₃)₂, and AA₂ is —CH₂CH₂CH₂NHC(O)NH₂.

An exemplary embodiment of the immunoconjugate of Formula I includes wherein AA₁ and AA₂ are independently selected from GlcNAc aspartic acid, —CH₂SO₃H, and —CH₂OPO₃H.

An exemplary embodiment of the immunoconjugate of Formula I includes wherein Bza is selected from Formulas Ia-d:

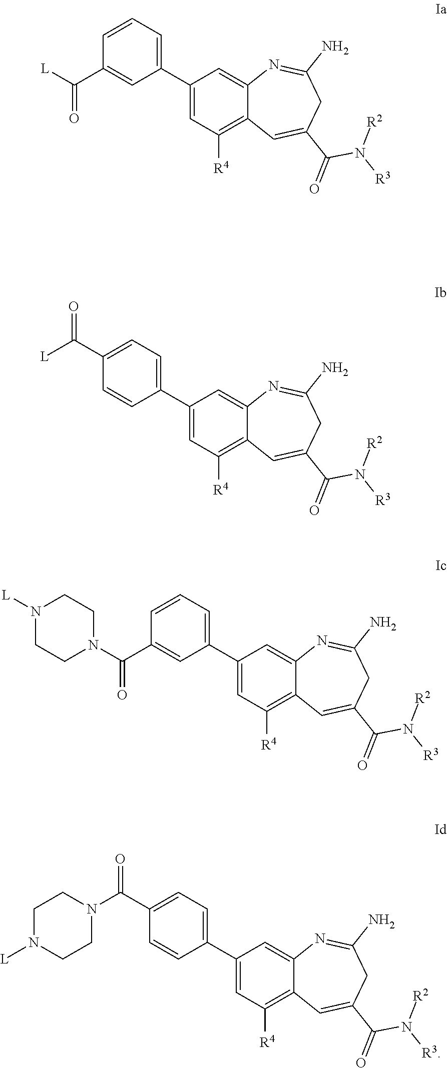

An exemplary embodiment of the immunoconjugate of Formula I includes wherein Bza is selected from Formulas Ie and If:

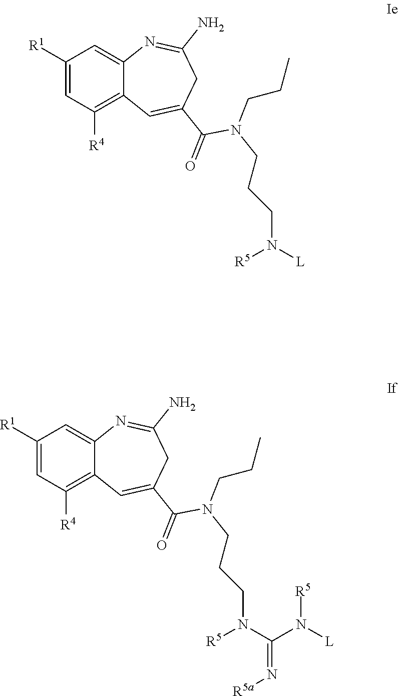

where R^{5a} of Formula If is phenyl, optionally substituted with one or more groups selected from F, Cl, Br, I, —CN, and —NO₂.

An exemplary embodiment of the immunoconjugate of Formula I includes wherein L is —C(═O)-(PEG)- or —C(═O)-(PEG)-C(═O)—.

An exemplary embodiment of the immunoconjugate of Formula I includes wherein Bza is selected from Formulas Ig and Ih:

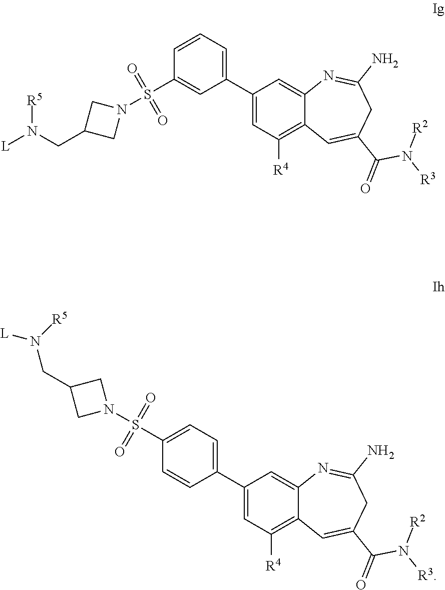

An exemplary embodiment of the immunoconjugate of Formula I includes wherein L is —C(═O)-(PEG)-C(═O)-(PEP)-.

An exemplary embodiment of the immunoconjugate of Formula I includes wherein R² and R³ are each C₁-C₅ alkyl.

An exemplary embodiment of the immunoconjugate of Formula I includes wherein R² and R³ are each —CH₂CH₂CH₃.

An exemplary embodiment of the immunoconjugate of Formula I includes wherein X² and X³ are each a bond, and R² or R³ is —O—(C₁-C₁₂ alkyl).

An exemplary embodiment of the immunoconjugate of Formula I includes wherein X² and X³ are each a bond, and R² or R³ is —OCH₂CH₃.

An exemplary embodiment of the immunoconjugate of Formula I includes wherein one of R¹ and R⁴ is selected from.
—(C₁-C₁₂ alkyldiyl)-N(R⁵)—*;
—(C₁-C₁₂ alkyldiyl)-N(R⁵)C(═NR⁵)N(R⁵)—*;
—(C₆-C₂₀ aryldiyl)-S(═O)₂—(C₂-C₂₀ heterocyclyldiyl)-*;
—(C₆-C₂₀ aryldiyl)-S(═O)₂—(C₂-C₂₀ heterocyclyldiyl)-(C₁-C₁₂ alkyldiyl)-N(R⁵)—*;
—(C₆-C₂₀ aryldiyl)-C(═O)—*;
—(C₆-C₂₀ aryldiyl)-(C₁-C₁₂ alkyldiyl)-N(R⁵)—*;
—(C₆-C₂₀ aryldiyl)-C(═O)—(C₂-C₂₀ heterocyclyldiyl)-*;
—C(═O)NR⁵—(C₁-C₂₀ heteroaryldiyl)-*; and
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*.

An exemplary embodiment of the immunoconjugate of Formula I includes wherein one of R² and R³ is selected from:
—(C₁-C₁₂ alkyldiyl)-N(R⁵)—*;
—(C₁-C₁₂ alkyldiyl)-O—(C₁-C₁₂ alkyldiyl)-N(R⁵)—*;
—(C₁-C₁₂ alkyldiyl)-N(R⁵)C(═NR⁵)—N(R⁵)—*;
—(C₁-C₁₂ alkyldiyl)-(C₆-C₂₀ aryldiyl)-(C₁-C₁₂ alkyldiyl)-N(R⁵)—*;
—(C₁-C₁₂ alkyldiyl)-(C₆-C₂₀ aryldiyl)-(C₁-C₁₂ alkyldiyl)-N(R⁵)—C(═NR⁵)N(R⁵)—*;
—(C₂-C₆ alkynyldiyl)-N(R⁵)—*; and
—(C₂-C₆ alkynyldiyl)-N(R⁵)C(═NR⁵)N(R⁵)—*;
X² and X³ are a bond, and where the asterisk * indicates the attachment site of L.

An exemplary embodiment of the immunoconjugate of Formula I includes wherein one of R¹ and R⁴ is selected from —(C₆-C₂₀ aryldiyl)-S(═O)₂—(C₂-C₂₀ heterocyclyldiyl)-(C₁-C₁₂ alkyldiyl)-N(R⁵)₂ and —(C₆-C₂₀ aryldiyl)-S(═O)₂—(C₂-C₂₀ heterocyclyldiyl)-(C₁-C₁₂ alkyldiyl)-OH.

An exemplary embodiment of the immunoconjugate of Formula I includes wherein C₆-C₂₀ aryldiyl is phenyldiyl and C₂-C₂₀ heterocyclyldiyl is azetidindiyl.

An exemplary embodiment of the immunoconjugate of Formula I includes wherein one of R¹ and R⁴ is selected from the formulas:

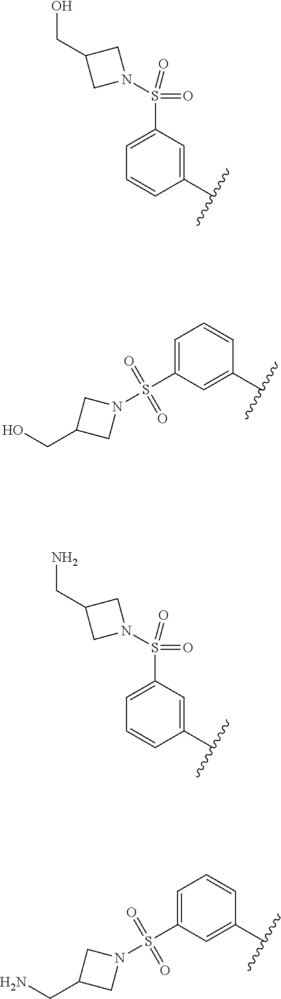 [SMILES: *c1cccc(S(=O)(=O)N2CC(CO)C2)c1 ; *c1cccc(S(=O)(=O)N2CC(CO)C2)c1 ; *c1cccc(S(=O)(=O)N2CC(CN)C2)c1 ; *c1cccc(S(=O)(=O)N2CC(CN)C2)c1]

An exemplary embodiment of the immunoconjugate of Formula I includes wherein one of R¹ and R⁴ is —C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵-L.

An exemplary embodiment of the immunoconjugate of Formula I includes wherein C₁-C₂₀ heteroaryldiyl is pyridindiyl and C₂-C₂₀ heterocyclyldiyl is piperidiyl.

In an exemplary embodiment, p is 1, 2, 3, or 4.

Exemplary embodiments of immunoconjugates comprise an antibody covalently attached to a linker which is covalently attached to one or more aminobenzazepine moieties, and having Formula III:

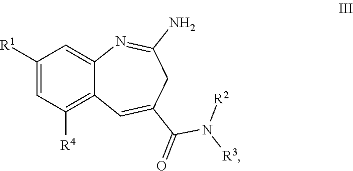

a pharmaceutically acceptable salt thereof, or a quaternary ammonium salt thereof,
wherein
R¹, R², R³, and R⁴ are independently Y or Z, wherein one of R¹, R², R³, and R⁴ is Y, having the formula:

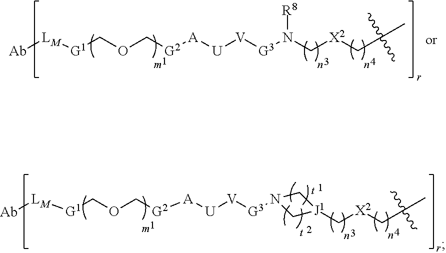

each Z independently is hydrogen or selected from the formulas:

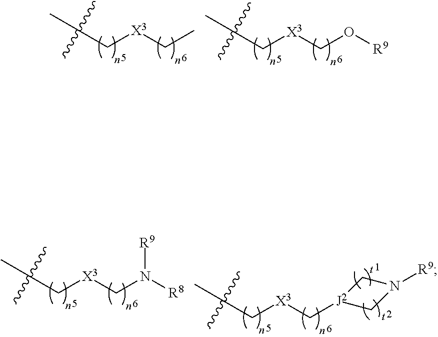

U is optionally present and is CH₂, C(═O), CH₂C(═O), or C(═O)CH_{2P}
A is optionally present and is NR¹⁰ or selected from the formulas:

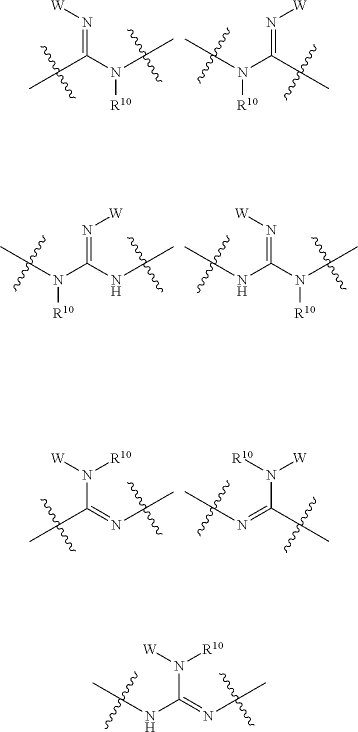

R¹⁰ and W independently are hydrogen, Ar¹, or of formula:

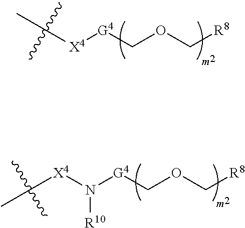

V is optionally present and is of formula:

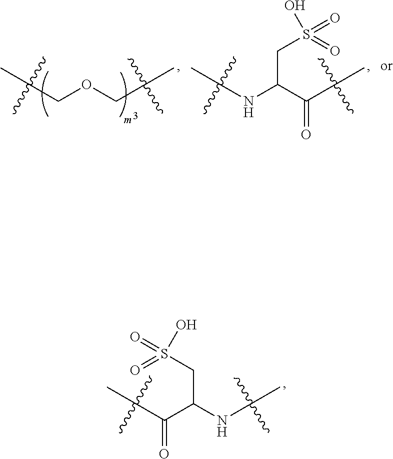

J¹ and J² independently are CH or N,
m¹, m², and m³ independently are an integer from 0 to 25, except that at least one of m¹, m², and m³ is a non-zero integer,
n¹, n², n³, n⁴, n⁵, and n⁶ independently are an integer from 0 to 10,
t¹ and t² independently are an integer from 1 to 3,
G¹, G², G³, and G⁴ independently are CH₂, C(═O), CH₂C(═O), C(═O)CH₂, or a bond,
X¹, X², X³, and X⁴ are each optionally present and independently are O, NR⁷, CHR⁷, SO₂, S, or one or two cycloalkyldiyl, heterocycloalkyldiyl, aryldiyl, or heteroaryldiyl groups, and when more than one cycloalkyldiyl, heterocycloalkyldiyl, aryldiyl, or heteroaryldiyl group is present, the more than one cycloalkyldiyl, heterocycloalkyldiyl, aryldiyl, or heteroaryldiyl groups are linked or fused, wherein linked cycloalkyldiyl, heterocycloalkyldiyl, aryldiyl, or heteroaryldiyl groups are linked through a bond or —CO—,
R⁹ is hydrogen, C₁-C₄ alkyl, or selected from the formulas:

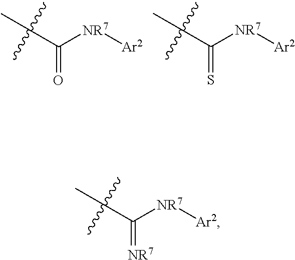

R⁸ is independently hydrogen or C₁-C₄ alkyl,
Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,
L_{M} is a linking moiety that comprises a functional group selected from an amide, amine, ester, carbamate, urea, thioether, thiocarbamate, thiocarbonate, and thiourea,
r is an integer from 1 to 10,
Ab is an antibody, and
each wavy line ( [symbol] ) represents a point of attachment.

An exemplary embodiment of the immunoconjugate of Formula III includes wherein subscript r is 1.

An exemplary embodiment of the immunoconjugate of Formula I or III includes wherein the antibody is an antibody construct that has an antigen binding domain that binds PD-L1.

An exemplary embodiment of the immunoconjugate of Formula I or III includes wherein the antibody is selected from the group consisting of atezolizumab, durvalumab, and avelumab, or a biosimilar or a biobetter thereof.

An exemplary embodiment of the immunoconjugate of Formula I or III includes wherein the antibody is an antibody construct that has an antigen binding domain that binds HER2.

An exemplary embodiment of the immunoconjugate of Formula I or III includes wherein the antibody is selected from the group consisting of trastuzumab and pertuzumab, or a biosimilar or a biobetter thereof.

An exemplary embodiment of the immunoconjugate of Formula I or III includes wherein the antibody is an antibody construct that has an antigen binding domain that binds CEA.

An exemplary embodiment of the immunoconjugate of Formula I or III includes wherein the antibody is selected from the group consisting of labetuzumab (also known as MN-14, hMN14, or CEA-CIDE™), PR1A3, MFE-23, SM3E, or a biosimilar or a biobetter thereof.

The invention includes all reasonable combinations, and permutations of the features, of the Formula I and III embodiments.

In certain embodiments, the immunoconjugate compounds of the invention include those with immunostimulatory activity. The antibody-drug conjugates of the invention selectively deliver an effective dose of an aminobenzazepine drug to tumor tissue, whereby greater selectivity (i.e., a lower efficacious dose) may be achieved while increasing the therapeutic index (“therapeutic window”) relative to unconjugated aminobenzazepine.

Drug loading is represented by p, the number of aminobenzazepine moieties per antibody in an immunoconjugate of Formula I or III. Drug (aminobenzazepine) loading may range from 1 to about 8 drug moieties (D) per antibody. Immunoconjugates of Formula I and III include mixtures or collections of antibodies conjugated with a range of drug moieties, from 1 to about 8. In some embodiments, the number of drug moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 5. In any such aspect, p and n are equal (i.e., p=n=1, 2, 3, 4, 5, 6, 7, or 8, or some range there between). Exemplary antibody-drug conjugates of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intrachain disulfide bonds, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

For some immunoconjugates, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with an aminobenzazepine-linker compound of Formula II. In certain embodiments, higher drug loading, e.g. p >5, may cause aggregation, insolubility, toxicity, or loss of cellular permeability of certain antibody-drug conjugates. In certain embodiments, the average drug loading for an immunoconjugate ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an immunoconjugate may be controlled in different ways, and for example, by: (i) limiting the molar excess of the aminobenzazepine-linker intermediate compound relative to antibody, (ii) limiting the conjugation reaction time or temperature, and (iii) partial or limiting reductive denaturing conditions for optimized antibody reactivity.

It is to be understood that where more than one nucleophilic group of the antibody reacts with a drug, then the resulting product is a mixture of antibody-drug conjugate compounds with a distribution of one or more drug moieties attached to an antibody. The average number of drugs per antibody may be calculated from the mixture by a dual ELISA antibody assay, which is specific for antibody and specific for the drug. Individual immunoconjugate molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al. (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous immunoconjugate with a single loading value may be isolated from the conjugation mixture by electrophoresis or chromatography.

An exemplary embodiment of the immunoconjugate of Formula I is selected from the Tables 3a, 3b, 3c Immunoconjugates.

TABLE 3a
Immunoconjugates (IC)

| Immuno- | BzL | | | Myeloid TNFα |
|---|---|---|---|---|
| conjugate | linker-adjuvant | Ab | | Secretion |
| No. | Table 2a | Antigen | DAR | EC50 nM |
| IC-1 | BzL-2 | Trastuzumab | 2.33 | >1000 |
| | | HER2 | | |
| IC-2 | BzL-3 | Trastuzumab | 2.06 | 14.8 |
| | | HER2 | | |
| IC-3 | BzL-4 | Trastuzumab | 2.05 | >1000 |
| | | HER2 | | |
| IC-4 | BzL-5 | Trastuzumab | 1.82 | >1000 |
| | | HER2 | | |
| IC-5 | BzL-7 | Trastuzumab | 1.6 | nd |
| | | HER2 | | |
| IC-6 | BzL-8 | Trastuzumab | 0.5 | nd |
| | | HER2 | | |
| IC-7 | BzL-9 | Trastuzumab | 1.6 | nd |
| | | HER2 | | |
| IC-8 | BzL-15 | Trastuzumab | 1.9 | 233.7 |
| | | HER2 | | |
| IC-9 | BzL-15 | Avelumab | 2.16 | 161.03 |
| | | PD-L1 | | |
| IC-10 | BzL-16 | Trastuzumab | 2.49 | >1000 |
| | | HER2 | | |
| IC-11 | BzL-17 | Trastuzumab | 1.84 | >1000 |
| | | HER2 | | |
| IC-12 | BzL-18 | Trastuzumab | 2.49 | >1000 |
| | | HER2 | | |
| IC-13 | BzL-19 | Trastuzumab | 2.05 | >1000 |
| | | HER2 | | |
| IC-14 | BzL-20 | Trastuzumab | 1.91 | >1000 |
| | | HER2 | | |
| IC-15 | BzL-21 | Avelumab | 2.85 | 199.5 |
| | | PD-L1 | | |
| IC-16 | BzL-21 | Trastuzumab | 1.74 | >1000 |
| | | HER2 | | |
| IC-17 | BzL-22 | Trastuzumab | 2.65 | >1000 |
| | | HER2 | | |
| IC-18 | BzL-25 | Trastuzumab | nd | nd |
| | | HER2 | | |
| IC-19 | BzL-27 | Trastuzumab | 1.61 | >1000 |
| | | HER2 | | |
| IC-20 | BzL-31 | Trastuzumab | 2.57 | 788 |
| | | HER2 | | |
| IC-21 | BzL-28 | Trastuzumab | 2.39 | >1000 |
| | | HER2 | | |

TABLE 3b
Immunoconjugates (IC)

| Immuno- | BzL | | | Myeloid |
|---|---|---|---|---|
| conjugate | linker-adjuvant | Ab | | TNFα Secretion |
| No. | Table 2b | Antigen | DAR | EC50 nM |
| IC-22 | BzL-33 | Trastuzumab | 2.37 | >1000 |
| | | HER2 | | |
| IC-23 | BzL-35 | Trastuzumab | 2.65 | 464 |
| | | HER2 | | |
| IC-24 | BzL-36 | Trastuzumab | 2.60 | >1000 |
| | | HER2 | | |
| IC-25 | Bzl-37 | Trastuzumab | 2.28 | >1000 |
| | | HER2 | | |
| IC-26 | Bzl-38 | Trastuzumab | 2.0 | 62 |
| | | HER2 | | |
| IC-27 | BzL-34 | Trastuzumab | 2.06 | 97 |
| | | HER2 | | |
| IC-28 | BzL-39 | Trastuzumab | 2.32 | >1000 |
| | | HER2 | | |
| IC-29 | BzL-40 | Trastuzumab | 2.95 | >1000 |
| | | HER2 | | |
| IC-30 | BzL-41 | Trastuzumab | 2.83 | 459 |
| | | HER2 | | |
| IC-31 | BzL-42 | Trastuzumab | 2.05 | 17.2 |
| | | HER2 | | |
| IC-32 | BzL-43 | Trastuzumab | 2.05 | 133 |
| | | HER2 | | |
| IC-33 | BzL-44 | Trastuzumab | 2.0 | 71 |
| | | HER2 | | |
| IC-34 | BzL-45 | Trastuzumab | 2.26 | 78 |
| | | HER2 | | |
| IC-35 | BzL-46 | Trastuzumab | 1.54 | 68 |
| | | HER2 | | |

TABLE 3c
Immunoconjugates (IC)

| Immuno- | BzL | | |
|---|---|---|---|
| conjugate | linker-adjuvant | Ab | |
| No. | Tables 2a-c | Antigen | DAR |
| IC-36 | BzL-40 | PDL1.24-G1f | 2.39 |
| IC-37 | BzL-39 | PDL1.24-G1f | 1.6 |
| IC-38 | Bzl-49 | Trastuzumab | 2.24 |
| | | HER2 | |
| IC-39 | BzL-35 | Rituximab | 2.40 |
| | | CD20 | |
| IC-40 | BzL-50 | Trastuzumab | 2.48 |
| | | HER2 | |
| IC-41 | BzL-51 | Trastuzumab | 2.57 |
| | | HER2 | |
| IC-42 | BzL-52 | Trastuzumab | 2.62 |
| | | HER2 | |
| IC-43 | BzL-53 | Trastuzumab | 2.18 |
| | | HER2 | |
| IC-44 | BzL-55 | Trastuzumab | 2.18 |
| | | HER2 | |
| IC-45 | BzL-56 | Trastuzumab | 1.96 |
| | | HER2 | |
| IC-46 | BzL-35 | anti-mPD-L1 | 2.27 |
| IC-47 | BzL-35 | rat IgG2b | 2.4 |
| | | isotype control | |
| IC-48 | Bzl-49 | PDL1.85-G1f | 2.21 |
| IC-49 | Bzl-49 | PDL1.85-G1f | 2.21 |
| IC-50 | BzL-54 | Trastuzumab | 2.13 |
| | | HER2 | 2.36 |
| IC-51 | Bzl-49 | CEA.5G1fhL2 | 2.35 |
| IC-52 | BzL-57 | Trastuzumab | 2.58 |
| | | HER2 | |
| IC-53 | BzL-60 | Trastuzumab | 2.11 |
| | | HER2 | |
| IC-54 | BzL-62 | Trastuzumab | 2.46 |
| | | HER2 | |
| IC-55 | BzL-58 | Trastuzumab | 2.35 |
| | | HER2 | |
| IC-56 | BzL-65 | Trastuzumab | 1.80 |
| | | HER2 | |
| IC-57 | BzL-35 | CEA.5G1fhL2 | 2.21 |
| IC-58 | BzL-35 | Tras-G1f-N297A | 2.34 |
| IC-59 | BzL-66 | Trastuzumab | 2.38 |
| | | HER2 | |
| IC-60 | BzL-67 | Trastuzumab | 2.15 |
| | | HER2 | 1.93 |
| IC-61 | BzL-68 | Trastuzumab | 2.36 |
| | | HER2 | |
| IC-62 | BzL-69 | Trastuzumab | 2.15 |
| | | HER2 | 2.99 |
| IC-63 | BzL-69 | Rituximab | 2.60 |
| | | CD20 | |
| IC-64 | BzL-69 | Tras-G1f-N297A | 2.41 |
| IC-65 | BzL-70 | Trastuzumab | 2.39 |
| | | HER2 | |
| IC-66 | BzL-72 | Trastuzumab | 2.39 |
| | | HER2 | |
| IC-67 | BzL-41 | CEA.9-G1fhL2 | 2.26 |
| IC-68 | BzL-35 | CEA.9-G1fhL2 | 2.37 |
| IC-69 | BzL-69 | CEA.9-G1fhL2 | 2.41 |
| IC-70 | BzL-63 | Trastuzumab | 2.24 |
| | | HER2 | |
| IC-71 | BzL-64 | Trastuzumab | 2.34 |
| | | HER2 | |
| IC-72 | BzL-35 | PDL1.24-G1f | 2.66 |
| IC-73 | BzL-35 | PDL1.85-G1f | 2.84 |
| IC-74 | BzL-73 | Trastuzumab | 2.17 |
| | | HER2 | |
| IC-75 | BzL-74 | Trastuzumab | 2.74 |
| | | HER2 | |
| IC-76 | BzL-77 | Trastuzumab | 2.43 |
| | | HER2 | |
| IC-77 | BzL-76 | Trastuzumab | 1.19 |
| | | HER2 | |
| IC-78 | BzL-78 | Trastuzumab | 2.10 |
| | | HER2 | |
| IC-79 | BzL-75 | Trastuzumab | 1.45 |
| | | HER2 | |
| IC-80 | BzL-69 | CEACAM5 | 1.84 |
| | | | 2.74 |
| IC-81 | BzL-77 | CEA.9-G1fhL2 | 2.39 |
| | | | 2.45 |
| IC-82 | BzL-72 | CEA.9-G1fhL2 | 2.70 |
| IC-83 | BzL-74 | CEA.9-G1fhL2 | 2.41 |
| IC-84 | BzL-80 | CEA.9-G1fhL2 | 1.81 |
| IC-85 | BzL-69 | PDL1.85-G1f | 2.69 |
| IC-86 | BzL-80 | Trastuzumab | 2.92 |
| | | HER2 | |
| IC-87 | BzL-82 | Trastuzumab | 2.56 |
| | | HER2 | |
| IC-88 | BzL-77 | PDL1.85-G1f | 2.55 |
| IC-89 | BzL-74 | PDL1.85-G1f | 2.68 |
| IC-90 | BzL-81 | Trastuzumab | 1.91 |
| | | HER2 | |
| IC-91 | BzL-85 | Trastuzumab | 2.18 |
| | | HER2 | |
| IC-92 | BzL-69 | Trastuzumab | 3.07 |
| | | HER2 | |

### Compositions of Immunoconjugates

The invention provides a composition, e.g., a pharmaceutically or pharmacologically acceptable composition or formulation, comprising a plurality of immunoconjugates as described herein and optionally a carrier therefor, e.g., a pharmaceutically or pharmacologically acceptable carrier. The immunoconjugates can be the same or different in the composition, i.e., the composition can comprise immunoconjugates that have the same number of adjuvants linked to the same positions on the antibody construct and/or immunoconjugates that have the same number of adjuvants linked to different positions on the antibody construct, that have different numbers of adjuvants linked to the same positions on the antibody construct, or that have different numbers of adjuvants linked to different positions on the antibody construct.

In an exemplary embodiment, a composition comprising the immunoconjugate compounds comprises a mixture of the immunoconjugate compounds, wherein the average drug loading per antibody in the mixture of immunoconjugate compounds is about 2 to about 5.

A composition of immunoconjugates of the invention can have an average adjuvant to antibody construct ratio of about 0.4 to about 10. A skilled artisan will recognize that the number of aminobenzazepine adjuvants conjugated to the antibody construct may vary from immunoconjugate to immunoconjugate in a composition comprising multiple immunoconjugates of the invention, and, thus, the adjuvant to antibody construct (e.g., antibody) ratio can be measured as an average, which may be referred to as the drug to antibody ratio (DAR). The adjuvant to antibody construct (e.g., antibody) ratio can be assessed by any suitable means, many of which are known in the art.

The average number of adjuvant moieties per antibody (DAR) in preparations of immunoconjugates from conjugation reactions may be characterized by conventional means such as mass spectrometry, ELISA assay, and HPLC. The quantitative distribution of immunoconjugates in a composition in terms of p may also be determined. In some instances, separation, purification, and characterization of homogeneous immunoconjugates where p is a certain value from immunoconjugates with other drug loadings may be achieved by means such as reverse phase HPLC or electrophoresis.

### Pharmaceutical Compositions and Methods of Administration

In other embodiments, another aspect of the invention relates to pharmaceutical compositions or dosage forms including therapeutically effective amount of an immunoconjugate of the invention and one or more pharmaceutically acceptable diluent, vehicle, carrier or excipient.

The pharmaceutical compositions can be any form that allows for administration to a patient. For example, the pharmaceutical composition can be in the form of a solid or liquid. Typical routes of administration include, without limitation, parenteral, ocular and intra-tumoral. Parenteral administration includes subcutaneous injections, intravenous, intramuscular or intrasternal injection or infusion techniques. In one embodiment, the compositions are administered parenterally. In a specific embodiment, the compositions are administered intravenously.

In some embodiments, the pharmaceutical composition further comprises one or more pharmaceutically or pharmacologically acceptable excipients. For example, the immunoconjugates of the invention can be formulated for parenteral administration, such as IV administration or administration into a body cavity or lumen of an organ. Alternatively, the immunoconjugates can be injected intra-tumorally. Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.10% (w/w) to about 10% (w/w).

Method of Treating Cancer with Immunoconjugates

The invention provides a method for treating cancer. The method includes administering a therapeutically effective amount of an immunoconjugate as described herein (e.g., as a pharmaceutical composition as described herein) to a subject in need thereof, e.g., a subject that has cancer and is in need of treatment for the cancer. The method includes administering a therapeutically effective amount of an immunoconjugate (IC) selected from Table 3.

It is contemplated that the immunoconjugate of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering an immunoconjugate containing an antibody construct that is capable of binding PD-L1 (e.g., atezolizumab, durvalumab, avelumab, biosimilars thereof, or biobetters thereof). In some embodiments, methods for treating breast cancer include administering an immunoconjugate containing an antibody construct that is capable of binding PD-L1 (e.g., atezolizumab, durvalumab, avelumab, biosimilars thereof, or biobetters thereof). In some embodiments, methods for treating triple-negative breast cancer include administering an immunoconjugate containing an antibody construct that is capable of binding PD-L1 (e.g., atezolizumab, durvalumab, avelumab, biosimilars thereof, or biobetters thereof).

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering an immunoconjugate containing an antibody construct that is capable of binding PD-L1 (e.g., atezolizumab, durvalumab, avelumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional therapeutic agent, such as a chemotherapeutic agent. Such combination therapies encompass combined administration (where two or more therapeutic agents are included in the same or separate formulations), and separate administration, in which case, administration of the immunoconjugate can occur prior to, simultaneously, and/or following, administration of the additional therapeutic agent and/or adjuvant. Immunoconjugates can also be used in combination with radiation therapy.

The immunoconjugates of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Atezolizumab, durvalumab, avelumab, biosimilars thereof, and biobetters thereof are known to be useful in the treatment of cancer, particularly breast cancer, especially triple negative (test negative for estrogen receptors, progesterone receptors, and excess HER2 protein) breast cancer, bladder cancer, and Merkel cell carcinoma. The immunoconjugate described herein can be used to treat the same types of cancers as atezolizumab, durvalumab, avelumab, biosimilars thereof, and biobetters thereof, particularly breast cancer, especially triple negative (test negative for estrogen receptors, progesterone receptors, and excess HER2 protein) breast cancer, bladder cancer, and Merkel cell carcinoma.

The immunoconjugate is administered to a subject in need thereof in any therapeutically effective amount using any suitable dosing regimen, such as the dosing regimens utilized for atezolizumab, durvalumab, avelumab, biosimilars thereof, and biobetters thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering an immunoconjugate containing an antibody construct that is capable of binding HER2 (e.g. trastuzumab, pertuzumab, biosimilars, or biobetters thereof) and PD-L1 (e.g., atezolizumab, durvalumab, avelumab, biosimilars, or biobetters thereof). In some embodiments, methods for treating colon cancer lung cancer, renal cancer, pancreatic cancer, gastric cancer, and esophageal cancer include administering an immunoconjugate containing an antibody construct that is capable of binding CEA, or tumors over-expressing CEA (e.g. labetuzumab, biosimilars, or biobetters thereof).

In some embodiments, the cancer is susceptible to a pro-inflammatory response induced by TLR7 and/or TLR8.

### EXAMPLES

### Preparation of aminobenzazepine compounds (Bz) and intermediates

### Example 1 Synthesis of Bz-1

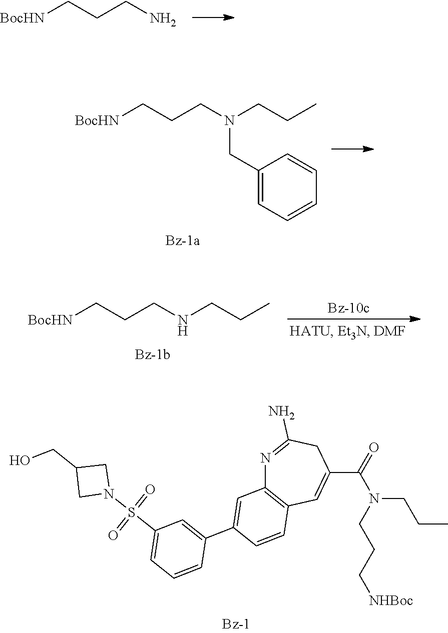 [SMILES: CC(C)(C)OC(=O)NCCCN ; CCCN(CCCNC(=O)OC(C)(C)C)Cc1ccccc1 ; CCCNCCCNC(=O)OC(C)(C)C ; CCCN(CCCNC(=O)OC(C)(C)C)C(=O)C1=Cc2ccc(-c3cccc(S(=O)(=O)N4CC(CO)C4)c3)cc2N=C(N)C1]

### Synthesis of tert-butyl (3-(benzyl(propyl)amino)propyl)carbamate Bz-1a

tert-Butyl N-(3-aminopropyl)carbamate (10 g, 57.39 mmol, 10.02 mL, 1 eq) and benzaldehyde (6.09 g, 57.39 mmol, 5.80 mL, 1 eq) in DCE (100 mL) was stirred at 70° C. for 24 hours. MeOH (100 mL) and NaBH₃CN (16.23 g, 258.26 mmol, 4.5 eq) was added to the mixture in portions at 0° C. The mixture was stirred at 0° C. for 2 hours, then propanal (16.67 g, 286.96 mmol, 20.89 mL, 5 eq) was added at 0° C. and stirred for 2 hours. LCMS showed the reaction was completed. The mixture was added a few drops water and concentrated in reduced pressure at 40° C. The residue was poured into ice water (200 mL) and stirred for 5 min. The aqueous phase was extracted with ethyl acetate (200 mL×3). The combined organic phase was washed with brine (300 mL), dried with anhydrous Na₂SO₄, filtered and concentrated in vacuum. The residue was purified by silica gel chromatography (Petroleum ether/Ethyl acetate=10/1, 3/1) to afford tert-butyl N-[3-[benzyl(propyl)amino]propyl]carbamate, Bz-1a (16 g, 52.21 mmol, 90.98% yield) as light yellow oil. ¹H NMR (CDCl₃, 400 MHz) δ 7.39-7.29 (m, 5H), 3.60-3.52 (m, 2H), 3.20-3.08 (m, 2H), 2.56-2.45 (m, 2H), 2.39 (s, 2H), 1.73-1.61 (m, 2H), 1.58-1.48 (m, 2H), 1.42 (s, 1H), 1.45 (s, 9H), 0.89 (t, J=7.2 Hz, 3H).

### Synthesis of tert-butyl N-[3-(propylamino)propyl]carbamate, Bz-1b

To a solution of tert-butyl N-[3-[benzyl(propyl)amino]propyl]carbamate, Bz-1a (10 g, 32.63 mmol, 1 eq) in MeOH (150 mL) was added Pd(OH)₂/C (10%, 3 g) under N₂. The suspension was degassed under vacuum and purged with H₂ several times. The mixture was stirred under H₂ (50 psi) at 50° C. for 12 hours. TLC (Petroleum ether/Ethyl acetate=3:1) showed the starting material was consumed completely. The reaction mixture was filtered and the filtrate was concentrated to give tert-butyl N-[3-(propylamino)propyl]carbamate, Bz-1b (5 g, 23.11 mmol, 70.83% yield) as colorless oil which was used into the next step without further purification. ¹H NMR (MeOD, 400 MHz) δ 3.13-3.05 (m, 2H), 2.60 (t, J=7.2 Hz, 2H), 2.56-2.50 (m, 2H), 1.66 (m, 2H), 1.58-1.48 (m, 2H), 1.44 (s, 9H), 0.94 (t, J=7.2 Hz, 3H).

### Synthesis of tert-butyl N-[3-[[2-amino-8-[3-[3-(hydroxymethyl)azetidin-1-yl]sulfonylphenyl]-3H-1-benzazepine-4-carbonyl]-propyl-amino]propyl]carbamate, Bz-1

To a mixture of tert-butyl N-[3-(propylamino)propyl]carbamate, Bz-1b (202.42 mg, 935.73 μmol (micromole), 2 eq) and 2-amino-8-[3-[3-(hydroxymethyl)azetidin-1-yl]sulfonylphenyl]-3H-1-benzazepine-4-carboxylic acid, Bz-10c from Example 6 (0.2 g, 467.87 μmol, 1 eq) in DMF (2 mL) was added HATU (213.48 mg, 561.44 μmol, 1.2 eq) and Et₃N (94.69 mg, 935.73 μmol, 130.24 μL (microliter), 2 eq) in one portion at 15° C. The mixture was stirred at 15° C. for 30 min. LCMS and HPLC showed the reaction was completed. The mixture was filtered and purified by prep-HPLC (column: Waters Xbridge 150×25 mm, 5 micron particle size; mobile phase: [water (10 mM NH₄HCO₃)-ACN]; B %: 30%-50%, 20 min) to afford tert-butyl N-[3-[[2-amino-8-[3-[3-(hydroxymethyl)azetidin-1-yl]sulfonylphenyl]-3H-1-benzazepine-4-carbonyl]-propyl-amino]propyl]carbamate, Bz-1 (0.087 g, 139.03 μmol, 29.72% yield) as light yellow solid. ¹H NMR (MeOD, 400 MHz) δ 8.07 (s, 1H), 8.03 (d, J=8.0 Hz, 1H), 7.86-7.81 (m, 1H), 7.79-7.73 (m, 1H), 7.50-7.45 (m, 2H), 7.39 (m, 1H), 6.92 (s, 1H), 3.86 (t, J=8.0 Hz, 2H), 3.61-3.58 (m, 2H), 3.52-3.48 (m, 2H), 3.45-3.41 (m, 4H), 3.10 (s, 4H), 2.62-2.52 (m, 1H), 1.86-1.79 (m, 2H), 1.71-1.65 (m, 2H), 1.42-1.50 (m, 9H), 0.87-0.95 (m, 3H). LC/MS [M+H]626.30 (calculated); LC/MS [M+H]626.40 (observed).

### Example 2 Synthesis of Bz-3

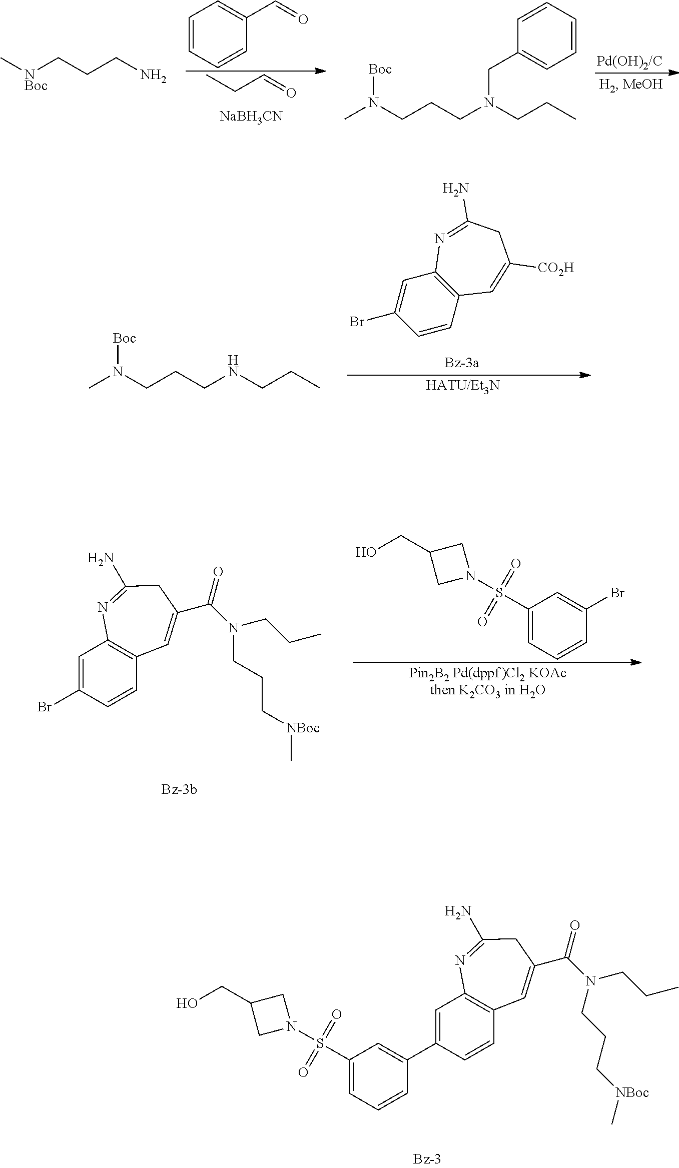

### Synthesis of tert-butyl (3-(benzyl(propyl)amino)propyl)(methyl)carbamate

To a mixture of benzaldehyde (310.02 mg, 2.92 mmol, 295.26 μL, 1 eq) in DCE (10 mL) was added tert-butyl N-(3-aminopropyl)-N-methyl-carbamate (0.55 g, 2.92 mmol, 1 eq) at 25° C. under N₂. The mixture was stirred at 60° C. for 12 hours, then cooled to 0° C., MeOH (10 mL) was added to the mixture, NaBH₃CN (550.48 mg, 8.76 mmol, 3 eq) was added to the mixture stirred for 1 hr. Propanal (339.18 mg, 5.84 mmol, 425.04 μL, 2 eq) was added to the mixture and stirred at 0° C. for 1 hr. LCMS showed the reaction was completed. The mixture was concentrated in vacuum. The residue was purified by prep-HPLC column: Luna C18 100×30 5u; mobile phase: [water(0.1% TFA)-ACN]; B %: 10%-40%, 10 min to give tert-butyl N-[3-[benzyl(propyl)amino]propyl]-N-methyl-carbamate (0.4 g, 1.25 mmol, 42.75% yield) as colorless oil. ¹H NMR (MeOD, 400 MHz) δ 7.18-7.37 (m, 5H), 3.57 (s, 2H), 3.20 (t, J=7.2 Hz, 2H), 2.78 (s, 3H), 2.35-2.52 (m, 4H), 1.70 (quin, J=7.2 Hz, 2H), 1.47-1.57 (m, 2H), 1.42 (s, 9H), 0.88 (t, J=7.2 Hz, 3H)

### Synthesis of tert-butyl methyl(3-(propylamino)propyl)carbamate

To a solution of tert-butyl N-[3-[benzyl(propyl)amino]propyl]-N-methyl-carbamate (0.4 g, 1.25 mmol, 1 eq) in MeOH (20 mL) was added Pd(OH)₂/C (0.2 g, 5% purity) under N₂. The suspension was degassed under vacuum and purged with H₂ several times. The mixture was stirred under H₂ (50 psi) at 50° C. for 12 hours. LCMS showed the reactant was consumed, desired mass was detected. The mixture was filtered and concentrated in vacuum. Afforded tert-butyl N-methyl-N-[3-(propylamino)propyl]carbamate (0.25 g, 1.09 mmol, 86.95% yield) as colorless oil. ¹H NMR (MeOD, 400 MHz) δ 3.26-3.31 (m, 2H), 2.85 (s, 3H), 2.56 (q, J=8.0 Hz, 4H), 1.74 (quin, J=7.2 Hz, 2H), 1.48-1.59 (m, 2H), 1.46 (s, 9H), 0.94 (t, J=7.2 Hz, 3H)

### Synthesis of tert-butyl (3-(2-amino-8-bromo-N-propyl-3H-benzo[b]azepine-4-carboxamido)propyl)(methyl)carbamate, Bz-3b

To a mixture of 2-amino-8-bromo-3H-1-benzazepine-4-carboxylic acid, Bz-3a (80 mg, 284.59 μmol, 1 eq) and tert-butyl N-methyl-N-[3-(propylamino)propyl]carbamate (78.67 mg, 341.51 μmol, 1.2 eq) in DMF (1 mL) was added HATU (162.32 mg, 426.89 μmol, 1.5 eq) Et₃N (57.60 mg, 569.18 μmol, 79.22 μL, 2 eq) at 25° C. under N₂. The mixture was stirred at 25° C. for 1 hr. LCMS showed major as desired. The mixture was poured into water (20 mL). The aqueous phase was extracted with ethyl acetate (20 mL×3). The combined organic phase was washed with brine (20 mL), dried with anhydrous Na₂SO₄, filtered and concentrated in vacuum. The residue was purified by prep-TLC (Petroleum ether/Ethyl acetate=0/1) to give Bz-3b (60 mg, 121.60 μmol, 42.73% yield) as yellow oil.

### Synthesis of tert-butyl (3-(2-amino-8-(3-((3-(hydroxymethyl)azetidin-1-yl)sulfonyl)phenyl)-N-propyl-3H-benzo[b]azepine-4-carboxamido)propyl)(methyl)carbamate, Bz-3

To a mixture of [1-(3-bromophenyl)sulfonylazetidin-3-yl]methanol (155.12 mg, 506.65 μmol, 1 eq) Pin₂B₂(154.39 mg, 607.98 μmol, 1.2 eq) potassium acetate, KOAc (124.31 mg, 1.27 mmol, 2.5 eq) in dioxane (30 mL) was added Pd(dppf)C₁₂·CH₂C₁₂ (41.38 mg, 50.67 μmol, 0.1 eq) at 25° C. under N₂. The mixture was stirred at 90° C. for 2 hours. tert-butyl N-[3-[(2-amino-8-bromo-3H-1-benzazepine-4-carbonyl)-propyl-amino]propyl]-N-methyl-carbamate, Bz-3b (0.25 g, 506.65 μmol, 1 eq) K₂CO₃ (140.04 mg, 1.01 mmol, 2 eq) in H₂O (2 mL) were added to the mixture, stirred at 90° C. for 2 hrs (hours) under nitrogen gas, N₂. LCMS showed the reaction was completed. The mixture was filtered and concentrated in vacuum. The residue was purified by prep-TLC (EtOAc/MeOH=7:1) to give Bz-3 (112 mg, 175.05 μmol, 34.55% yield) as a light yellow solid. ¹H NMR (MeOD, 400 MHz) δ 8.07 (s, 1H), 8.03 (d, J=7.6 Hz, 1H), 7.85 (br d, J=7.6 Hz, 1H), 7.73-7.79 (m, 1H), 7.41-7.54 (m, 3H), 6.95 (s, 1H), 3.86 (t, J=8.2 Hz, 2H), 3.60 (dd, J=8.0, 6.0 Hz, 2H), 3.39-3.52 (m, 6H), 3.17-3.29 (m, 2H), 2.82-2.90 (m, 4H), 2.53-2.67 (m, 1H), 1.89-1.92 (m, 2H), 1.66-1.72 (m, 2H), 1.42-1.46 (m, 9H), 0.80-1.05 (m, 3H). LC/MS [M+H]640.32 (calculated); LC/MS [M+H]640.30 (observed).

### Example 3 Synthesis of Bz-5

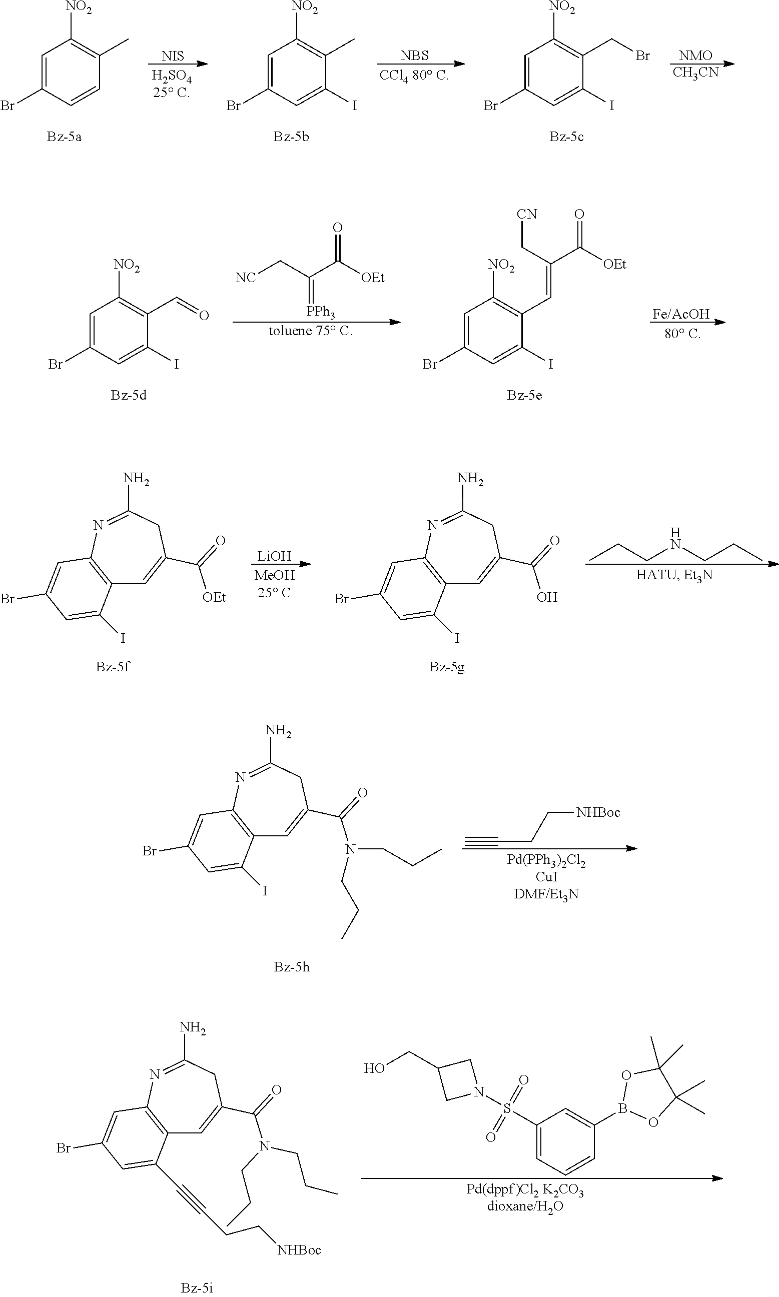

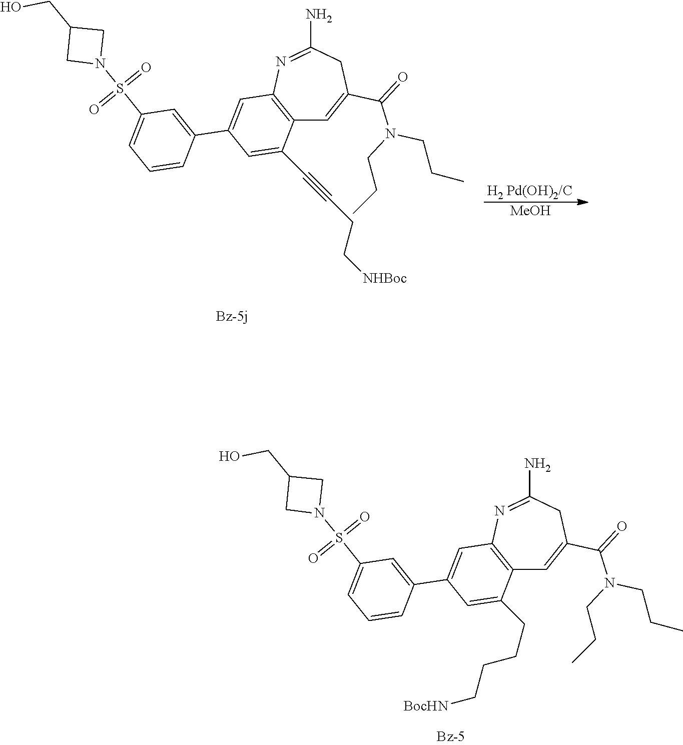 [SMILES: "MeOH" ; CCCN(CCC)C(=O)C1=Cc2c(C#CCCNC(=O)OC(C)(C)C)cc(-c3cccc(S(=O)(=O)N4CC(CO)C4)c3)cc2N=C(N)C1 ; CCCN(CCC)C(=O)C1=Cc2c(CCCCNC(=O)OC(C)(C)C)cc(-c3cccc(S(=O)(=O)N4CC(CO)C4)c3)cc2N=C(N)C1]

### Example 3 Synthesis of Bz-5

To a mixture of 4-bromo-1-methyl-2-nitro-benzene, Bz-5a (20 g, 92.58 mmol, 20.00 mL, 1 eq) in H₂SO₄ (20 mL) was added NIS (37.49 g, 166.64 mmol, 1.8 eq) at 0° C. under N₂. The mixture was stirred at 0° C. for 1 hour. TLC showed the reactant was consumed and two points formed. The mixture was poured into ice-water (200 mL) slowly. The aqueous phase was extracted with ethyl acetate (150 mL×2). The combined organic phase was washed with brine (150 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=100/1, 20/1) to afford Bz-5b (14 g, 40.94 mmol, 44.23% yield) as white solid. ¹H NMR (CDCl₃, 400 MHz) δ 8.20 (d, J=2.0 Hz, 1H), 7.87 (d, J=2.0 Hz, 1H), 2.55 (s, 3H).

### Synthesis of 5-bromo-2-(bromomethyl)-1-iodo-3-nitrobenzene, Bz-5c

To a mixture of 5-bromo-1-iodo-2-methyl-3-nitro-benzene, Bz-5b (13 g, 38.02 mmol, 1 eq) in CCl₄ (100 mL) was added NBS (10.15 g, 57.03 mmol, 1.5 eq) BPO (920.94 mg, 3.80 mmol, 0.1 eq) at 25° C. under N₂. The mixture was stirred at 90° C. for 12 hours. TLC showed one new point formed, HPLC and LCMS showed about 50% as desired and about 50% the reactant remained. The mixture was concentrated in vacuum. The residue was purified by silica gel chromatography (column height: 250 mm, diameter: 100 mm, 100-200 mesh silica gel, Petroleum ether/Ethyl acetate=50/1, 10/1) to afford Bz-5c (7 g, 16.63 mmol, 43.75% yield) as white solid. ¹H NMR (CDCl₃-d₆, 400 MHz) δ 8.29 (d, J=2.0 Hz, 1H), 8.02 (d, J=2.0 Hz, 1H), 4.82 (s, 3H).

### Synthesis of 4-bromo-2-iodo-6-nitrobenzaldehyde, Bz-5d

To a mixture of 5-bromo-2-(bromomethyl)-1-iodo-3-nitro-benzene, Bz-5c (7 g, 16.63 mmol, 1 eq) in CH₃CN (10 mL) was added NMO (3.90 g, 33.27 mmol, 3.51 mL, 2 eq) at 25° C. under N₂. The mixture was stirred at 25° C. for 2 hours. TLC showed the reaction was completed. The mixture was concentrated in vacuum. The residue was purified by silica gel chromatography (column height: 250 mm, diameter: 100 mm, 100-200 mesh silica gel, Petroleum ether/Ethyl acetate=20/1, 4/1) to afford Bz-5d (5 g, 14.05 mmol, 84.46% yield) as white solid. ¹H NMR (CDCl₃, 400 MHz) δ 10.00 (s, 1H), 8.37 (d, J=1.6 Hz, 1H), 8.15 (d, J=1.6 Hz, 1H)

### Synthesis of (E)-ethyl 3-(4-bromo-2-iodo-6-nitrophenyl)-2-(cyanomethyl)acrylate, Bz-5e

To a mixture of 4-bromo-2-iodo-6-nitro-benzaldehyde, Bz-5d (3.5 g, 9.83 mmol, 1 eq) in toluene (30 mL) was added ethyl 3-cyano-2-(triphenyl-phosphanylidene)propanoate (5.71 g, 14.75 mmol, 1.5 eq) at 25° C. under N₂. The mixture was stirred at 85° C. for 12 hours. TLC showed major as desired. The mixture was concentrated in vacuum. The residue was purified by silica gel chromatography (column height: 250 mm, diameter: 100 mm, 100-200 mesh silica gel, Petroleum ether/Ethyl acetate=10/1, 1/1) to afford Bz-5e (2 g, 4.30 mmol, 43.73% yield) as yellow oil. ¹H NMR (CDCl₃, 400 MHz) δ 8.62 (d, J=1.8 Hz, 1H), 8.42 (d, J=1.8 Hz, 1H), 7.74 (s, 1H), 4.32 (q, J=7.2 Hz, 2H), 3.33 (s, 2H), 1.31 (t, J=7.2 Hz, 3H)

### Synthesis of ethyl 2-amino-8-bromo-6-iodo-3H-benzo[b]azepine-4-carboxylate, Bz-5f

To a mixture of ethyl (E)-3-(4-bromo-2-iodo-6-nitro-phenyl)-2-(cyanomethyl)prop-2-enoate, Bz-5e (2 g, 4.30 mmol, 1 eq) in acetic acid, AcOH (20 mL) was added Fe (1.20 g, 21.50 mmol, 5 eq) at 25° C. under N₂. The mixture was stirred at 80° C. for 5 hours. LCMS showed major as desired and the reactant was consumed. The reaction was filtered and the filtrate was concentrated in vacuum. The residue was purified by silica gel chromatography (column height: 250 mm, diameter: 100 mm, 100-200 mesh silica gel, Petroleum ether/Ethyl acetate=1/1, 0/1) to afford Bz-5f (1.8 g, 4.14 mmol, 96.20% yield) as off-white solid. ¹H NMR (DMSO-d₆, 400 MHz) δ 7.71 (s, 1H), 7.69 (d, J=2.0 Hz, 1H), 7.22 (br d, J=2.0 Hz, 1H), 4.26 (q, J=7.0 Hz, 3H), 2.83 (s, 2H), 1.30 (t, J=7.2 Hz, 3H).

### Synthesis of 2-amino-8-bromo-6-iodo-3H-benzo[b]azepine-4-carboxylic acid, Bz-5g

To a mixture of ethyl 2-amino-8-bromo-6-iodo-3H-1-benzazepine-4-carboxylate, Bz-5f (1.8 g, 4.14 mmol, 1 eq) in EtOH (40 mL) was added LiOH·H₂O (1.04 g, 24.82 mmol, 6 eq) in H₂O (10 mL) at 25° C. under N₂. The mixture was stirred at 35° C. for 2 hours. LCMS showed the reaction was completed. The mixture was concentrated to remove the EtOH, then adjusted PH to 5 by aq HCl (4M), filtered to get desired solid to afford Bz-5g (1.2 g, 2.95 mmol, 71.26% yield) as white solid. ¹H NMR (DMSO-d₆, 400 MHz) δ 7.77 (s, 1H), 7.69 (s, 1H), 7.29 (s, 1H), 2.92 (s, 2H)

### Synthesis of 2-amino-8-bromo-6-iodo-N,N-dipropyl-3H-benzo[b]azepine-4-carboxamide, Bz-5h

To a mixture of N-propylpropan-1-amine (186.47 mg, 1.84 mmol, 254.04 μL, 1.5 eq) and 2-amino-8-bromo-6-iodo-3H-1-benzazepine-4-carboxylic acid, Bz-5g (0.5 g, 1.23 mmol, 1 eq) in DMF (10 mL) was added HATU (700.67 mg, 1.84 mmol, 1.5 eq) Et₃N (186.47 mg, 1.84 mmol, 256.49 μL, 1.5 eq) at 25° C. The mixture was stirred at 25° C. for 30 min. LCMS showed the reaction was completed. The mixture was poured into water (50 mL), separated out from the mixture, and filtered to obtain Bz-5h (0.55 g, 1.12 mmol, 91.33% yield) as yellow solid. ¹H NMR (DMSO-d₆, 400 MHz) δ 7.74 (d, J=2.0 Hz, 1H), 7.33 (d, J=2.0 Hz, 1H), 6.81 (s, 1H), 3.43-3.47 (m, 4H), 1.66-1.72 (m, 4H), 0.93 (s, 6H)

### Synthesis of tert-butyl (4-(2-amino-8-bromo-4-(dipropylcarbamoyl)-3H-benzo[b]azepin-6-yl)but-3-yn-1-yl)carbamate, Bz-5i

To a mixture of 2-amino-8-bromo-6-iodo-N,N-dipropyl-3H-1-benzazepine-4-carboxamide, Bz-5h (200 mg, 408.02 μmol, 1 eq) and tert-butyl N-but-3-ynylcarbamate (72.50 mg, 428.42 μmol, 1.05 eq) in DMF (5 mL) Et₃N (1 mL) was added Pd(PPh₃)₂C₁₂ (14.32 mg, 20.40 μmol, 0.05 eq) Et₃N (0.5 mL) CuI (15.54 mg, 81.60 μmol, 0.2 eq) at 25° C. under N₂. The mixture was stirred at 80° C. for 1 hours. LCMS showed major as desired. The mixture was poured into water (20 mL). The aqueous phase was extracted with ethyl acetate (20 mL×3). The combined organic phase was washed with brine (20 mL), dried with anhydrous Na₂SO₄, filtered and concentrated in vacuum. The residue was purified by prep-TLC(Petroleum ether/Ethyl acetate=0/1) to give Bz-5i (0.2 g, 376.31 μmol, 92.23% yield) as a yellow solid. ¹H NMR (CDCl₃, 400 MHz) δ 7.40 (s, 1H), 7.35 (s, 1H), 7.13 (s, 1H), 3.46-3.52 (m, 4H), 3.35-3.40 (m, 2H), 2.65 (s, 2H), 1.58-1.78 (m, 4H), 1.46 (s, 9H), 0.93 (t, J=7.2 Hz, 6H)

### Synthesis of tert-butyl (4-(2-amino-4-(dipropylcarbamoyl)-8-(3-((3-(hydroxymethyl)azetidin-1-yl)sulfonyl)phenyl)-3H-benzo[b]azepin-6-yl)but-3-yn-1-yl)carbamate, Bz-5j

To a mixture of tert-butyl N-[4-[2-amino-8-bromo-4-(dipropylcarbamoyl)-3H-1-benzazepin-6-yl]but-3-ynyl]carbamate, Bz-5i (0.18 g, 338.67 μmol, 1 eq) and [1-[3-(4,4,5,5-tetramethyl-1,3,2-dioxaborolan-2-yl)phenyl]sulfonylazetidin-3-yl]methanol (179.45 mg, 508.01 μmol, 1.5 eq) in dioxane (10 mL) H₂O (1 mL) was added Pd(dppf)C₁₂ (12.39 mg, 16.93 μmol, 0.05 eq) K₂CO₃ (93.61 mg, 677.35 μmol, 2 eq) at 25° C. under N₂. The mixture was stirred at 90° C. for 2 hours. LCMS showed desired mass was detected. The mixture was concentrated in vacuum to give Bz-5j (0.2 g, crude) as a yellow solid.

### Synthesis of tert-butyl (4-(2-amino-4-(dipropylcarbamoyl)-8-(3-((3-(hydroxymethyl)azetidin-1-yl)sulfonyl)phenyl)-3H-benzo[b]azepin-6-yl)butyl)carbamate, Bz-5

To a solution of tert-butyl N-[4-[2-amino-4-(dipropylcarbamoyl)-8-[3-[3-(hydroxymethyl)azetidin-1-yl]sulfonylphenyl]-3H-1-benzazepin-6-yl]but-3-ynyl]carbamate, Bz-5j (140 mg, 206.53 μmol, 1 eq) in MeOH (20 mL) was added Pd(OH)₂/C (0.1 g, 5% purity) under N₂. The suspension was degassed under vacuum and purged with H₂ several times. The mixture was stirred under H₂ (50 psi) at 25° C. for 2 hours. LCMS showed the reaction was completed. The mixture was filtered and concentrated in vacuum. The residue was purified by prep-HPLC column: Xtimate C18 150×25 mm, 5 micron particle size; mobile phase: [water(0.040% NH₃H₂O+10 mM NH₄HCO₃)-ACN]; B %: 50%-60%, 10.5 min. Afforded Bz-5 (45 mg, 65.99 μmol, 31.95% yield) as a white solid. ¹H NMR (MeOD, 400 MHz) δ 8.00-8.08 (m, 2H), 7.83 (d, J=7.6 Hz, 1H), 7.71-7.79 (m, 1H), 7.33 (s, 1H), 7.28 (s, 1H), 6.99 (s, 1H), 3.86 (t, J=8.0 Hz, 2H), 3.57-3.66 (m, 2H), 3.38-3.51 (m, 6H), 3.06 (t, J=6.4 Hz, 2H), 2.84 (t, J=7.6 Hz, 2H), 2.52-2.63 (m, 1H), 1.50-1.77 (m, 8H), 1.41 (s, 9H), 0.94 (s, 6H). LC/MS [M+H]682.36 (calculated); LC/MS [M+H]682.40 (observed).

### Example 4 Synthesis of Bz-6

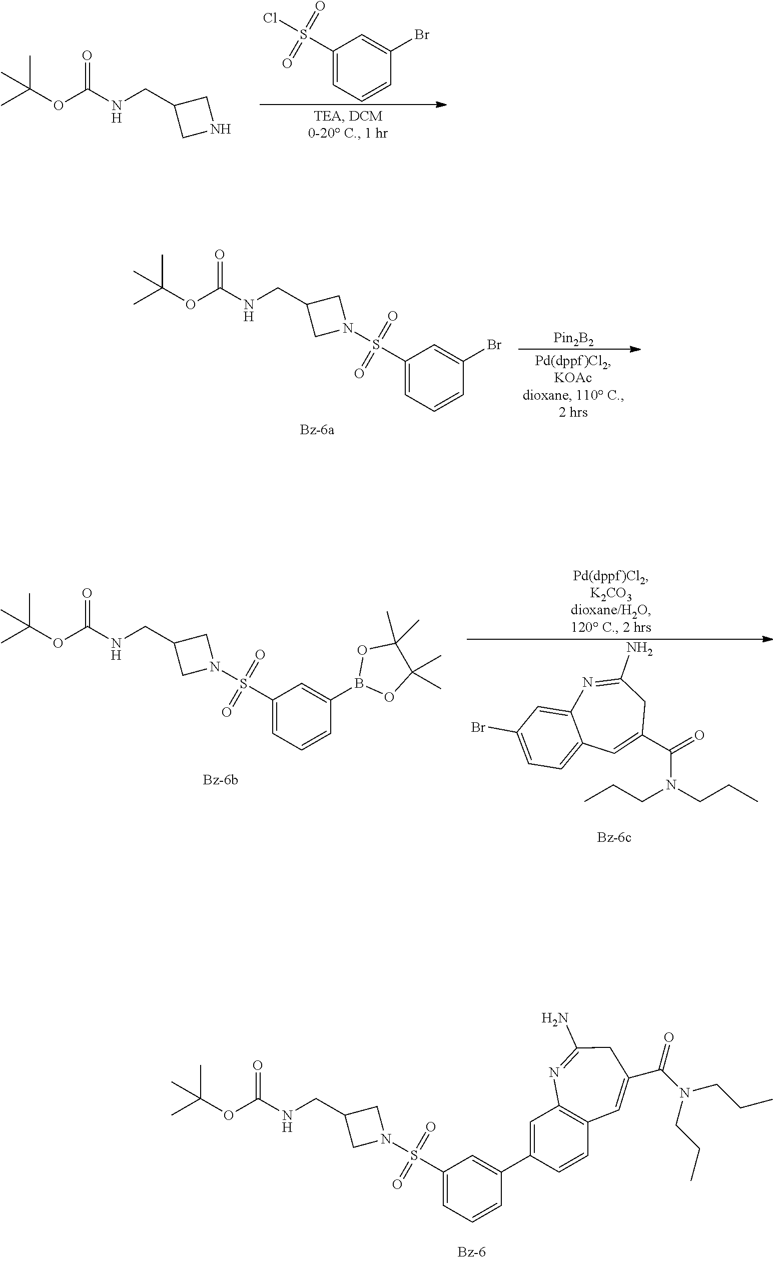 [SMILES: CC(C)(C)OC(=O)NCC1CNC1 ; O=S(=O)(Cl)c1cccc(Br)c1 ; CC(C)(C)OC(=O)NCC1CN(S(=O)(=O)c2cccc(Br)c2)C1 ; CC(C)(C)OC(=O)NCC1CN(S(=O)(=O)c2cccc(B3OC(C)(C)C(C)(C)O3)c2)C1 ; CCCN(CCC)C(=O)C1=Cc2ccc(Br)cc2N=C(N)C1 ; CCCN(CCC)C(=O)C1=Cc2ccc(-c3cccc(S(=O)(=O)N4CC(CNC(=O)OC(C)(C)C)C4)c3)cc2N=C(N)C1]

### Synthesis of tert-butyl ((1-((3-bromophenyl)sulfonyl)azetidin-3-yl)methyl)carbamate, Bz-6a

To a mixture of tert-butyl N-(azetidin-3-ylmethyl)carbamate (1.6 g, 8.59 mmol, 1.2 eq) in DCM (5 mL) was added TEA (1.45 g, 14.32 mmol, 1.99 mL, 2 eq) and 3-bromobenzenesulfonyl chloride (1.83 g, 7.16 mmol, 1.03 mL, 1 eq) at 0° C. The mixture was stirred at 20° C. for 1 hr. The mixture was diluted with water (50 mL) and extracted with DCM (25 ml×3). The organic layer was washed with brine (25 mL), dried over Na₂SO₄, filtered and concentrated. The residue was purified by flash silica gel chromatography (ISCO®; 4 g SepaFlash® Silica Flash Column, Eluent of 0˜100% Ethyl acetate/Petroleum ether gradient @35 mL/min). Compound tert-butyl N-[[1-(3-bromophenyl)sulfonylazetidin-3-yl]methyl]carbamate, Bz-6a (2.5 g, 6.17 mmol, 86.16% yield) was obtained as white solid. ¹H NMR (CDCl₃, 400 MHz) δ 7.99 (t, J=4.0 Hz, 1H), 7.74-7.81 (m, 2H), 7.47 (t, J 8.0 Hz, 1H), 4.61 (s, 1H), 3.86 (t, J=8.0 Hz, 2H), 3.50-3.58 (m, 2H), 3.19 (t, J=4.0 2H), 2.58-2.70 (m, 1H), 1.42 (s, 9H).

### Preparation of tert-butyl N-[[1-[3-(4,4,5,5-tetramethyl-1,3,2-dioxaborolan-2-yl)phenyl]sulfonylazetidin-3-yl]methyl]carbamate, Bz-6b

To a mixture of tert-butyl-N-[[1-(3-bromophenyl)sulfonylazetidin-3-yl]methyl]carbamate, Bz-6a (1 g, 2.47 mmol, 1 eq) in dioxane (10 mL) was added Pin₂B₂(939.80 mg, 3.70 mmol, 1.5 eq) and KOAc (484.29 mg, 4.93 mmol, 2 eq), Pd(dppf)C₁₂ (90.27 mg, 123.36 μmol, 0.05 eq) at 15° C. under N₂. The mixture was stirred at 110° C. for 2 hrs. The product tert-butyl N-[[1-[3-(4,4,5,5-tetramethy 1-1,3,2-dioxaborolan-2-yl)phenyl]sulfonylazetidin-3-yl]methyl]carbamate, Bz-6b was not isolated and used into next step.

### Synthesis of tert-butyl ((1-((3-(2-amino-4-(dipropylcarbamoyl)-3H-benzo[b]azepin-8-yl)phenyl)sulfonyl)azetidin-3-yl)methyl)carbamate, Bz-6

To a mixture of tert-butyl N-[[1-[3-(4,4,5,5-tetramethyl-1,3,2-dioxaborolan-2-yl) phenyl]sulfonylazetidin-3-yl]methyl]carbamate, Bz-6b (1.12 g, 2.48 mmol, 1 eq) and 2-amino-8-bromo-N,N-dipropyl-3H-1-benzazepine-4-carboxamide, Bz-6c (901.90 mg, 2.48 mmol, 1 eq) in dioxane (3 mL) was added K₂CO₃ (684.35 mg, 4.95 mmol, 2 eq) and Pd(dppf)C₁₂ (90.58 mg, 123.79 μmol, 0.05 eq) at 15° C. under N₂. The mixture was stirred at 120° C. for 2 hrs. The mixture was filtered and concentrated. The residue was purified by flash silica gel chromatography (ISCO®; 2 g SepaFlash® Silica Flash Column, Eluent of 0˜100% Ethyl acetate/Petroleum ether gradient @60 mL/min) to give Bz-6 (600 mg, 983.97 μmol, 39.74% yield, 100% purity) as yellow solid. ¹H NMR (MeOD-d₄, 400 MHz) δ 7.99-8.10 (m, 2H), 7.74-7.86 (m, 2H), 7.36-7.52 (m, 3H), 6.89 (s, 1H), 3.83 (t, J=8.0 Hz, 2H), 3.54 (t, J=8.0 Hz, 2H), 3.34-3.48 (m, 6H), 3.02 (d, J=8.0 Hz, 2H), 2.48-2.64 (m, 1H), 1.59-1.76 (m, 4H), 1.37 (s, 9H), 0.96-0.89 (m, 6H). LC/MS [M+H]610.31 (calculated); LC/MS [M+H]610.40 (observed).

### Example 5 Synthesis of Bz-9

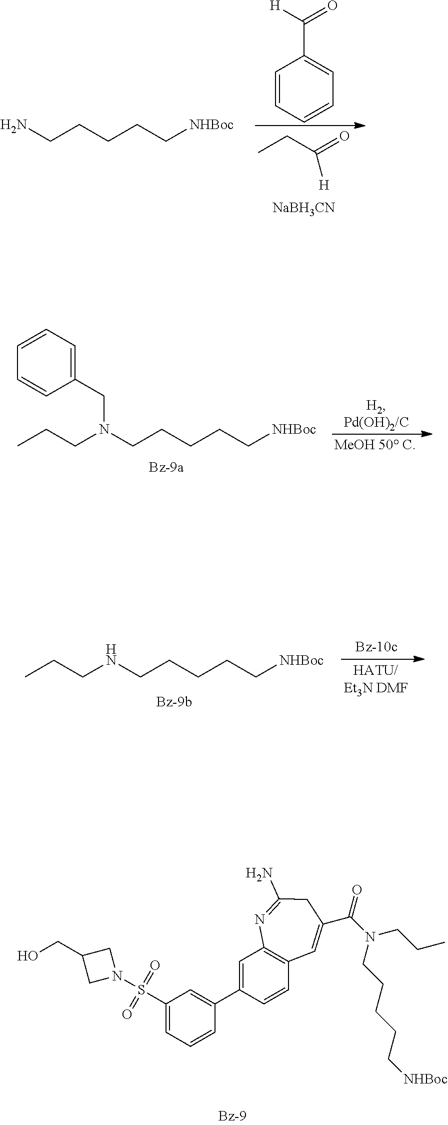

### Synthesis of tert-butyl (5-(benzyl(propyl)amino)pentyl)carbamate Bz-9a

To a mixture of tert-butyl N-(5-aminopentyl)carbamate (1 g, 4.94 mmol, 1.03 mL, 1 eq) and benzaldehyde (524.59 mg, 4.94 mmol, 499.61 μL, 1 eq) in DCE (10 mL) and stirred at 60° C. for 12 h. Then the mixture was cooled to 0° C. and MeOH (10 mL) was added to the mixture. NaBH₃CN (931.94 mg, 14.83 mmol, 3 eq) was added to the mixture and stirred for 1 h at 0° C. Propanal (574.20 mg, 9.89 mmol, 719.55 μL, 2 eq) was added to the mixture and stirred for 1 h. LCMS showed the reaction was finished. The mixture was concentrated. The residue was further purification by prep-HPLC(column: Luna C18 100×30, 5 micron particle size; mobile phase: [water(0.1% TFA)-ACN]; B %: 25%-40%, 10 min) to give tert-butyl-N-[5-[benzyl(propyl)amino]pentyl]carbamate Bz-9a (0.5 g, 1.49 mmol, 30.24% yield) as a yellow oil. ¹H NMR (400 MHz, METHANOL-d₄) δ=7.33-7.28 (m, 3H), 7.27-7.19 (m, 1H), 3.58 (s, 2H), 3.00 (t, J=7.2 Hz, 2H), 2.47-2.37 (m, 4H), 1.58-1.46 (m, 6H), 1.47 (s, 9H) 1.37-1.20 (m, 3H), 0.87 (t, J=7.6 Hz, 3H)

### Synthesis of tert-butyl (5-(propylamino)pentyl)carbamate Bz-9b

To a solution of tert-butyl N-[5-[benzyl(propyl)amino]pentyl]carbamate Bz-9a (0.5 g, 1.49 mmol, 1 eq) in MeOH (20 mL) was added Pd(OH)₂/C (0.2 g, 5% purity) at 25° C. under N₂. The suspension was degassed under vacuum and purged with H₂ several times. The mixture was stirred under H₂ (50 psi) at 50° C. for 12 hours. LCMS showed the reaction was finished. The mixture was filtered and concentrated. To give the product tert-butyl N-[5-(propylamino)pentyl]carbamate Bz-9b (0.3 g, crude) as colorless oil. ¹H NMR (400 MHz, METHANOL-d₄) δ=3.03 (t, J=6.8 Hz, 2H), 2.55 (d, J=7.6, 13.6 Hz, 4H), 1.59-1.44 (m, 6H), 1.47 (s. 9H)1.43-1.20 (m, 2H), 0.97-0.88 (m, 3H).

To a mixture of tert-butyl N-[5-(propylamino)pentyl]carbamate Bz-9b (57.17 mg, 233.93 μmol, 1 eq) and 2-amino-8-[3-[3-(hydroxymethyl)azetidin-1-yl]sulfonylphenyl]-3H-1-benzazepine-4-carboxylic acid Bz-10c (0.1 g, 233.93 μmol, 1 eq) in DMF (4 mL) was added HATU (133.42 mg, 350.90 μmol, 1.5 eq) and Et₃N (71.02 mg, 701.80 μmol, 97.68 μL, 3 eq) in one portion at 25° C. The mixture was stirred at 25° C. for 0.5 h. LCMS showed the reaction was finished. The mixture was diluted with water and extracted with EA (30 ml×3). The organic layer was washed with brine, dried over Na₂SO₄, filtered and concentrated. The residue was further purification by pre-HPLC(column: Xtimate C₁₈ 150×25 mm, 5 micron particle size; mobile phase: [water(0.1% TFA)-ACN]; B %: 32%-62%, 10.5 min) to give tert-butyl N-[5-[[2-amino-8-[3-[3-(hydroxymethyl) azetidin-1-yl]sulfonylphenyl]-3H-1-benzazepine-4-carbonyl]-propyl-amino]pentyl]carbamate Bz-9 (0.128 g, 179.48 μmol, 76.72% yield, 91.68% purity) as yellow solid. ¹H NMR (400 MHz, METHANOL-d₄) δ=8.10 (s, 1H), 8.07 (d, J=7.6 Hz, 1H), 7.89 (d, J=7.8 Hz, 1H), 7.83-7.78 (m, 1H), 7.77-7.65 (m, 3H), 7.09 (s, 1H), 3.86 (t, J=8.2 Hz, 2H), 3.61 (J=5.6, 8.0 Hz, 2H), 3.56-3.35 (m, 8H), 3.31 (s, 2H), 3.10-2.99 (m, 2H), 2.64-2.53 (m, 1H), 1.80-1.59 (m, 4H), 1.57-1.47 (m, 2H), 1.40 (s, 9H), 1.03-0.86 (m, 3H). LC/MS [M+H]654.33 (calculated); LC/MS [M+H]654.50 (observed).

### Example 6 Synthesis of Bz-10

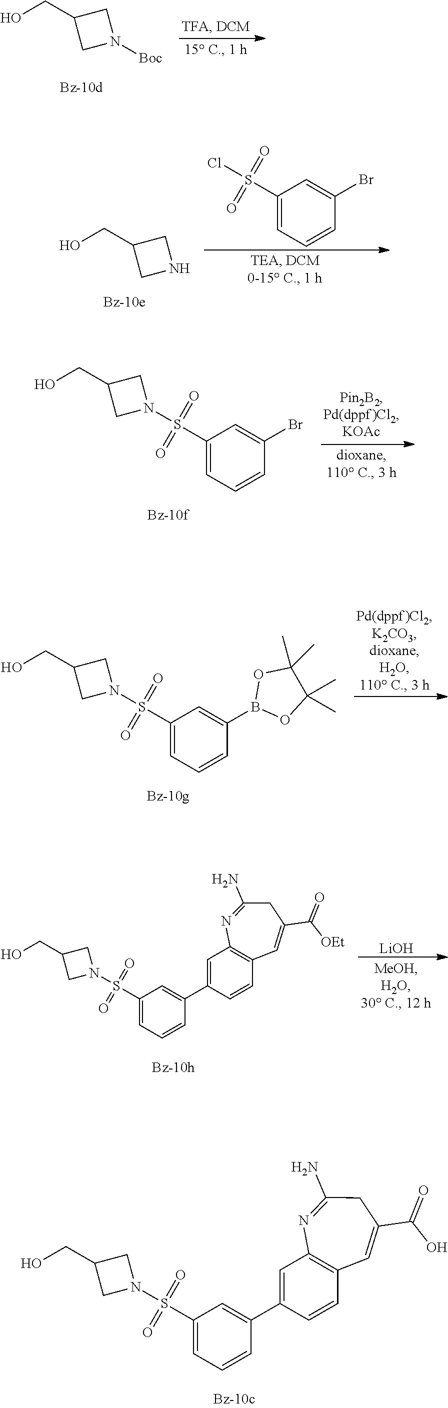

Preparation of Bz-10c: To a mixture of tert-butyl 3-(hydroxymethyl)azetidine-1-carboxylate Bz-10d (15 g, 80.11 mmol) in DCM (100 mL) was added TFA (63.94 g, 560.79 mmol, 41.52 mL, 7 eq) at 15° C. The mixture was stirred at 15° C. for 1 h. The mixture was concentrated to give azetidin-3-ylmethanol Bz-10e (36 g, crude, TFA) as yellow oil. ¹H NMR (DMSO-d₆, 400 MHz) δ 4.50-4.56 (m, 2H), 3.94-4.10 (m, 2H), 3.80-3.93 (m, 2H), 3.15-3.30 (m, 1H).

Preparation of [1-(3-bromophenyl)sulfonylazetidin-3-yl]methanol, Bz-1Of: To a mixture of azetidin-3-ylmethanol (33.06 g, 164.37 mmol, 2 eq, TFA) and 3-bromobenzenesulfonyl chloride (21 g, 82.19 mmol, 11.86 mL, 1 eq) in DCM (200 mL) was added TEA (33.27 g, 328.75 mmol, 45.76 mL, 4 eq) at 0° C. The mixture was stirred at 15° C. for 1 h. The residue was poured into saturated sodium bicarbonate in aqueous solution (200 mL) and stirred 10 min. The aqueous phase was extracted with DCM (100 mL×3). The combined organic phase was washed with brine (100 mL), dried with anhydrous Na₂SO₄, filtered and concentrated in vacuum. The residue was purified by flash silica gel chromatography (ISCO®; 1 g SepaFlash® Silica Flash Column, Eluent of 0˜100% Ethyl acetate/Petroleum ether gradient at 50 mL/min). Compound [1-(3-bromophenyl)sulfonylazetidin-3-yl]methanol Bz-10f (21 g, 68.59 mmol, 83.45% yield) was obtained as white solid. ¹H NMR (CDCl₃, 400 MHz) δ 7.89-8.11 (m, 1H), 7.78 (dd, J=8.0, 2.0 Hz, 2H), 7.39-7.54 (m, 1H), 3.78-3.97 (m, 2H), 3.49-3.74 (m, 4H), 2.41-2.77 (m, 1H).

Preparation of [1-[3-(4,4,5,5-tetramethyl-1,3,2-dioxaborolan-2-yl)phenyl]sulfonylazetidin-3-yl]methanol, Bz-10g: To a mixture of [1-(3-bromophenyl)sulfonylazetidin-3-yl]methanol (8 g, 26.13 mmol, 1 eq) in dioxane (10 mL) was added Pin₂B₂(9.95 g, 39.19 mmol, 1.5 eq), KOAc (5.13 g, 52.26 mmol, 2 eq) and Pd(dppf)C₁₂ (1.91 g, 2.61 mmol, 0.1 eq) at 15° C. The mixture was stirred at 110° C. for 3 h. LC-MS showed reactant 1 was consumed completely and one main peak with desired mass was detected. The mixture was filtered, washed by using ethyl acetate. Then the filtrate was concentrated in vacuum. The residue was purified by silica gel chromatography (column height: 250 mm, diameter: 100 mm, 100-200 mesh silica gel, Petroleum ether/Ethyl acetate=1/1, 0/1) to give 12 g crude product. The crude product was triturated with heptane/methyl tertiary butyl ether=5/1(50 mL), filtered, the filter cake was dried in vacuum. Compound [1-[3-(4,4,5,5-tetramethyl-1,3,2-dioxaborolan-2-yl)phenyl]sulfonylazetidin-3-yl]methanol (8.2 g, 23.21 mmol, 88.84% yield) was obtained as pink solid. ¹H NMR (CDCl₃, 400 MHz) δ 8.28 (s, 1H), 8.06 (d, J=8.0 Hz, 1H), 7.89-7.95 (m, 1H), 7.58 (t, J=8.0 Hz, 1H), 3.87 (t, J=8.0 Hz, 2H), 3.62-3.68 (m, 4H), 2.55-2.65 (m, 1H), 1.37 (s, 12H).

Preparation of ethyl 2-amino-8-[3-[3-(hydroxymethyl)azetidin-1-yl]sulfonylphenyl]-3H-1-benzazepine-4-carboxylate, Bz-10h: To a mixture of [1-[3-(4,4,5,5-tetramethyl-1,3,2-dioxaborolan-2-yl)phenyl]sulfonylazetidin-3-yl]methanol, Bz-10g (4.11 g, 11.64 mmol, 1.2 eq) and ethyl 2-amino-8-bromo-3H-1-benzazepine-4-carboxylate (3 g, 9.70 mmol, 1 eq) in dioxane (40 mL) and H₂O (3 mL) was added K₂CO₃ (2.68 g, 19.41 mmol, 2 eq) and Pd(dppf)C₁₂ (355.02 mg, 485.19 μmol, 0.05 eq) at 15° C. under N₂. The mixture was stirred at 110° C. for 3 h. LC-MS showed reactant 1 was consumed completely and one main peak with desired mass was detected. The mixture was concentrated. The crude product was triturated with EtOAc/H₂O=1:1 (200 mL) at 0° C. for 10 min and filtered, the filter cake was dried in vacuum. Compound ethyl 2-amino-8-[3-[3-(hydroxymethyl)azetidin-1-yl]sulfonylphenyl]-3H-1-benzazepine-4-carboxylate, Bz-10h (4 g, crude) was obtained as a white solid. ¹H NMR (DMSO-d₆, 400 MHz) δ 8.06-8.15 (m, 1H), 7.96 (s, 1H), 7.71-7.85 (m, 3H), 7.57 (d, J=8.0 Hz, 1H), 7.29-7.38 (m, 2H), 6.94 (s, 2H), 4.17-4.30 (m, 2H), 3.77 (t, J=8.0 Hz, 2H), 3.49 (t, J=8.0 Hz, 2H), 3.2 (d, J=8.0 Hz, 2H), 2.93 (s, 2H), 2.43-2.49 (m, 1H), 1.31 (t, J=8.0 Hz, 3H).

### 2-Amino-8-[3-[3-(hydroxymethyl)azetidin-1-yl]sulfonylphenyl]-3H-1-benzazepine-4-carboxylic acid, Bz-10c

To a solution of ethyl 2-amino-8-[3-[3-(hydroxymethyl)azetidin-1-yl]sulfonylphenyl]-3H-1-benzazepine-4-carboxylate, Bz-10h (4 g, 8.78 mmol, 1 eq) in MeOH (50 mL) and H₂O (10 mL) was added LiOH·H₂O (1.84 g, 43.91 mmol, 5 eq). The mixture was stirred at 30° C. for 12 h. LC-MS showed reactant 1 was consumed completely and one main peak with desired mass was detected. The reaction mixture was concentrated under reduced pressure to remove MeOH. The mixture was filtered. The filtrate was adjusted pH to around 6 by progressively adding a solution of HCl (1 M) and then filtered to give crude product. The crude product was triturated with CH₃CN (100 mL) at 0° C. for 10 min. The product was dried in vacuum. Compound 2-amino-8-[3-[3-(hydroxymethyl)azetidin-1-yl]sulfonylphenyl]-3H-1-benzazepine-4-carboxylic acid, Bz-10c (2.51 g, 5.72 mmol, 65.11% yield, 97.375% purity) was obtained as a gray solid. ¹H NMR (DMSO-d₆, 400 MHz) δ 8.11-8.16 (m, 1H), 8.02 (s, 1H), 7.92 (s, 1H), 7.78-7.88 (m, 4H), 7.75 (s, 1H), 3.76 (t, J=8.0 Hz, 2H), 3.45-3.54 (m, 4H), 3.20 (d, J=4.0 Hz, 2H), 2.45-2.49 (m, 1H). LC/MS [M+H]428.13 (calculated); LC/MS [M+H]428.20 (observed).

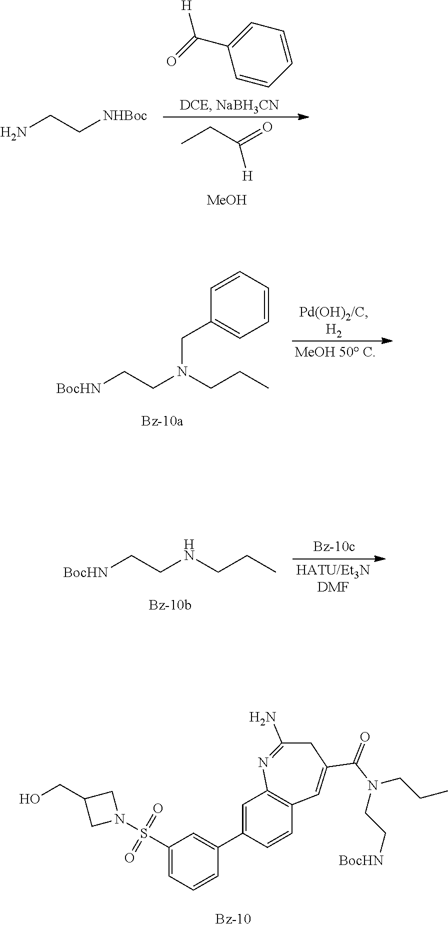

### Synthesis of tert-butyl N-[2-[benzyl(propyl)amino]ethyl]carbamate Bz-10a

To a mixture of benzaldehyde (2 g, 18.85 mmol, 1.90 mL, 1 eq) and tert-butyl N-(2-aminoethyl)carbamate (3.32 g, 20.73 mmol, 3.26 mL, 1.1 eq) in DCE (30 mL) was added NaBH₃CN (2.37 g, 37.69 mmol, 2 eq) at 0° C. The mixture was stirred at 0° C. for 30 min, propanal (5.47 g, 94.23 mmol, 6.86 mL, 5 eq) was added to the mixture and stirred for 1 hour at 25° C. The mixture was poured into ice water (50 mL) and the aqueous phase was extracted with ethyl acetate (50 mL×3). The combined organic phase was washed with brine (50 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=5/1, 1/1) to afford tert-butyl N-[2-[benzyl(propyl)amino]ethyl]carbamate Bz-10a (3 g, 10.26 mmol, 54.44% yield) as a colorless oil.

### Synthesis of tert-butyl N-[2-(propylamino)ethyl]carbamate Bz-10b

To a solution of tert-butyl N-[2-[benzyl(propyl)amino]ethyl]carbamate (2 g, 6.84 mmol, 1 eq) in MeOH (50 mL) was added Pd(OH)₂/C (10%, 1 g) under N₂. The suspension was degassed under vacuum and purged with H₂ several times. The mixture was stirred under H₂ (50 psi) at 50° C. for 12 hours. TLC (Petroleum ether/Ethyl acetate=3:1) showed the starting material was consumed completely. The reaction mixture was filtered and the filtrate was concentrated to give the crude product tert-butyl N-[2-(propylamino)ethyl]carbamate (1.3 g, 6.43 mmol, 93.96% yield) as colorless oil which was used into the next step without further purification. ¹H NMR (MeOD, 400 MHz) δ 3.18 (t, J=6.0 Hz, 2H), 2.68 (t, J=6.0 Hz, 2H), 2.56 (t, J=8.0 Hz, 2H), 1.58-1.48 (m, 2H), 1.44 (s, 9H), 0.94 (t, J=8.0 Hz, 3H).

### Synthesis of tert-butyl (2-(2-amino-8-(3-((3-(hydroxymethyl)azetidin-1-yl)sulfonyl)phenyl)-N-propyl-3H-benzo[b]azepine-4-carboxamido)ethyl)carbamate, Bz-10

To a mixture of 2-amino-8-[3-[3-(hydroxymethyl)azetidin-1-yl]sulfonylphenyl]-3H-1-benzazepine-4-carboxylic acid, Bz-10c (0.15 g, 350.90 μmol, 1 eq) and tert-butyl-N-[2-(propylamino)ethyl]carbamate (141.97 mg, 701.80 μmol, 2 eq) in DMF (4 mL) was added HATU (160.11 mg, 421.08 μmol, 1.2 eq), Et₃N (106.52 mg, 1.05 mmol, 146.52 μL, 3 eq) in one portion at 25° C. The mixture was stirred at 25° C. for 12 h. LCMS showed the reaction was finished. The mixture was filtered and purified by prep-HPLC (column: Waters Xbridge 150×25 5 u; mobile phase: [water (10 mM NH₄HCO₃)-ACN]; B %: 25%-45%, 20 min) to give tert-butyl N-[2-[[2-amino-8-[3-[3-(hydroxymethyl)azetidin-1-yl]sulfonylphenyl]-3H-1-benzazepine-4-carbonyl]-propyl-amino]ethyl]carbamate (0.036 g, 55.05 μmol, 15.69% yield, 93.54% purity) as yellow solid. ¹H NMR (MeOD, 400 MHz) δ 8.07 (s, 1H), 8.03 (d, J=7.6 Hz, 1H), 7.86-7.81 (d, J=8.0 Hz, 1H), 7.78-7.73 (m, 1H), 7.47 (s, 2H), 7.41-7.36 (m, 1H), 6.95 (s, 1H), 3.86 (t, J=8.4 Hz, 2H), 3.62-3.53 (m, 4H), 3.49-3.44 (m, 2H), 3.41 (d, J=6.4 Hz, 2H), 3.32-3.29 (m, 3H), 2.63-2.51 (m, 1H), 1.68 (d, J=7.2 Hz, 2H), 1.43 (s, 9H), 0.98-0.83 (m, 3H). LC/MS [M+H]612.29 (calculated); LC/MS [M+H]612.40 (observed).

### Example 7 Synthesis of Bz-11

### Synthesis of 2-amino-N-(3-aminopropyl)-8-[3-[3-(hydroxymethyl)azetidin-1-yl]sulfonylphenyl]-N-propyl-3H-1-benzazepine-4-carboxamide, Bz-11a

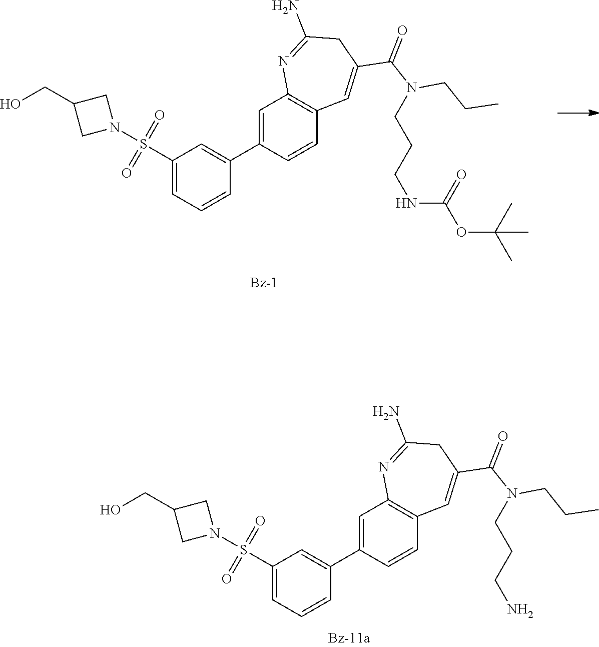 [SMILES: CCCN(CCCNC(=O)OC(C)(C)C)C(=O)C1=Cc2ccc(-c3cccc(S(=O)(=O)N4CC(CO)C4)c3)cc2N=C(N)C1 ; CCCN(CCCN)C(=O)C1=Cc2ccc(-c3cccc(S(=O)(=O)N4CC(CO)C4)c3)cc2N=C(N)C1]

To a mixture of tert-butyl N-[3-[[2-amino-8-[3-[3-(hydroxymethyl)azetidin-1-yl]sulfonylphenyl]-3H-1-benzazepine-4-carbonyl]-propyl-amino]propyl]carbamate, Bz-1 (0.5 g, 799.01 μmol, 1 eq) in DCM (20 mL) was added TFA (1.82 g, 15.98 mmol, 1.18 mL, 20 eq) in one portion at 15° C. The mixture was stirred at 15° C. for 3 hours. LCMS showed the reactant was consumed. The mixture was concentrated in vacuum, the residue was poured into ice water (30 mL) and adjusted pH=11 with Na₂CO₃.aq. The aqueous phase was extracted with DCM/i-PrOH=3/1 (20 mL×3). The combined organic phase was washed with brine (10 mL), dried with anhydrous Na₂SO₄, filtered and concentrated in vacuum. The crude product 2-amino-N-(3-aminopropyl)-8-[3-[3-(hydroxymethyl)azetidin-1-yl]sulfonylphenyl]-N-propyl-3H-1-benzazepine-4-carboxamide, Bz-11a (0.4 g, crude) as yellow oil which was used into the next step without further purification.

### Synthesis of 2-amino-N-[3-(tert-butylcarbamoylamino)propyl]-8-[3-[3-(hydroxymethyl)azetidin-1-yl]sulfonylphenyl]-N-propyl-3H-1-benzazepine-4-carboxamide, Bz-11

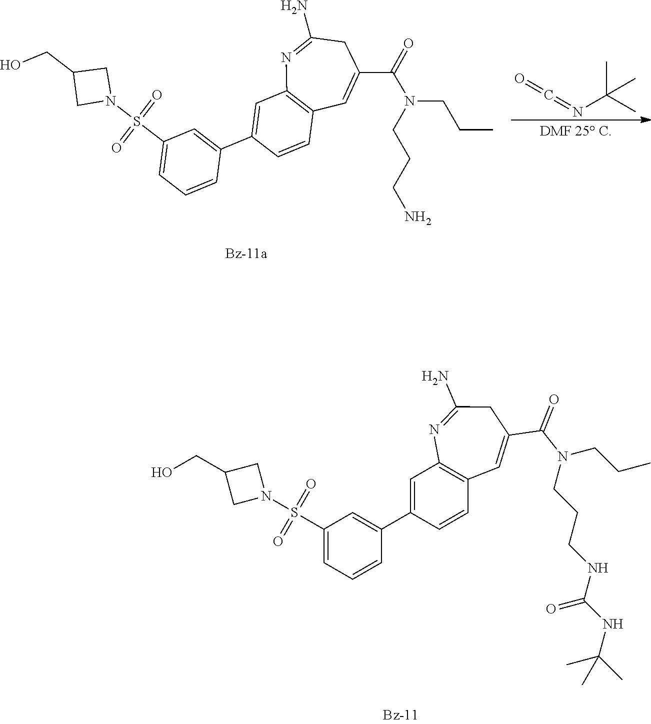

To a solution of 2-amino-N-(3-aminopropyl)-8-[3-[3-(hydroxymethyl)azetidin-1-yl]sulfonylphenyl]-N-propyl-3H-1-benzazepine-4-carboxamide, Bz-11a (0.1 g, 190.24 μmol, 1 eq) in DMF (2 mL) was added 2-isocyanato-2-methyl-propane (18.86 mg, 190.24 μmol, 22.45 μL, 1 eq) in one portion at 15° C. The mixture was stirred at 15° C. for 12 hours. LCMS showed the reaction was completed. The mixture was filtered and purified by prep-HPLC (column: Nano-micro Kromasil® (Nouryon) C18 100×30 mm, 5 micron particle size; mobile phase: [water (0.10% TFA)-ACN]; B %: 25%-45%, 10 min) to give crude product, then purified by prep-HPLC (column: Welch Xtimate C18 150×25 mm, 5 micron particle size; mobile phase: [water (10 mM NH₄HCO₃)-ACN]; B %: 25%-65%, 10.5 min) to give Bz-11 (0.007 g, 11.20 μmol, 5.89% yield) as light yellow solid. ¹H NMR (MeOD, 400 MHz) δ 8.09 (s, 1H), 8.05 (d, J=8.0 Hz, 1H), 7.87-7.85 (m, 1H), 7.80-7.76 (m, 1H), 7.51-7.49 (m, 2H), 7.43-7.41 (m, 1H), 6.94 (s, 1H), 3.88 (t, J=8.0 Hz, 2H), 3.63-3.60 (m, 2H), 3.54-3.50 (m, 2H), 3.44-3.43 (m, 4H), 3.15-2.91 (m, 4H), 2.67-2.58 (m, 1H), 1.84-1.79 (m, 2H), 1.73-1.66 (m, 2H), 1.40-1.14 (m, 9H), 1.00-0.90 (m, 3H).

### Example 8 Synthesis of Bz-12

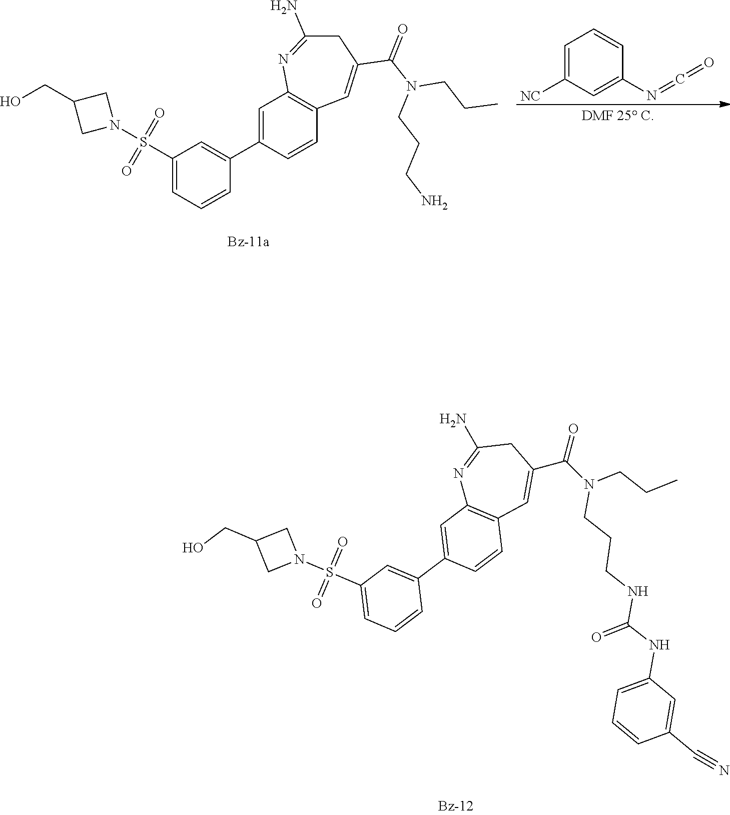

To a solution of 2-amino-N-(3-aminopropyl)-8-[3-[3-(hydroxymethyl)azetidin-1-yl]sulfonylphenyl]-N-propyl-3H-1-benzazepine-4-carboxamide, Bz-11a (0.1 g, 190.24 μmol, 1 eq) in DMF (0.3 mL) was added 3-isocyanatobenzonitrile (27.42 mg, 190.24 μmol, 1 eq) in one portion at 15° C. The mixture was stirred at 15° C. for 12 hours. LCMS showed the reaction was completed. The mixture was filtered and purified by prep-HPLC (column: Nano-micro Kromasil C18 100×30 mm Sum; mobile phase: [water(0.10% TFA)-ACN]; B %: 250%-45%, 10 min) to give 2-amino-N-[3-[(3-cyanophenyl)carbamoylamino]propyl]-8-[3-[3-(hydroxymethyl)azetidin-1-yl]sulfonylphenyl]-N-propyl-3H-1-benzazepine-4-carboxamide, Bz-12 (10 mg, 14.93 μmol, 7.85% yield) as yellow solid. ¹HNMR (CD₃OD, 400 MHz) i 8.21-7.88 (m, 4H), 7.86-7.80 (m, 1H), 7.68 (s, 3H), 7.59-7.24 (m, 3H), 7.15 (s, 1H), 3.89 (t, J=8.0 Hz, 2H), 3.64 (i, 4H), 3.51 (s, 2H, 3.46 (d, J=6.0 Hz, 2H), 3.40 (s, 2H), 3.30-3.19 (m, 2H), 2.63-2.60 (m, 1H), 1.96-1.92 (m, 2H), 1.77-1.71 (m, 2H), 1.07-0.86 (m, 3H).

### Example 9 Synthesis of Bz-13

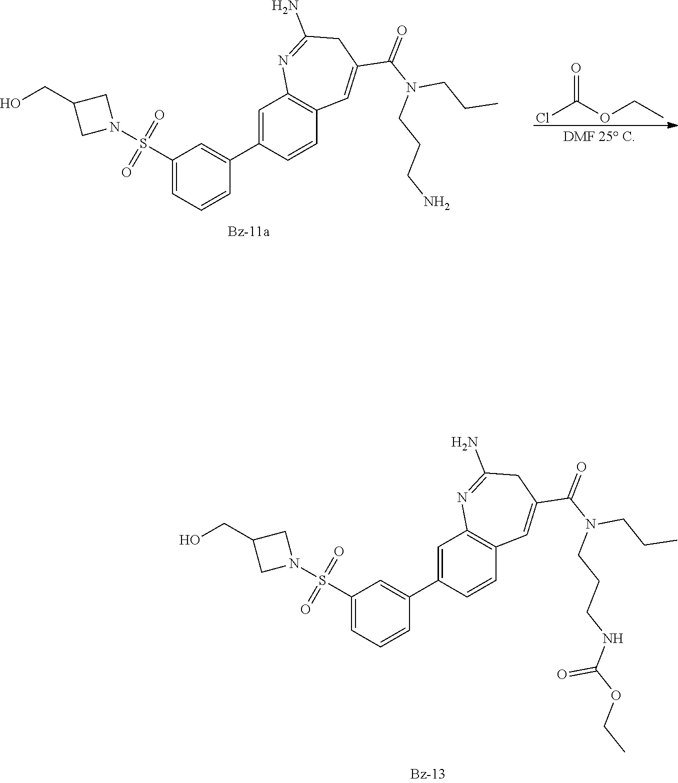

To a mixture of 2-amino-N-(3-aminopropyl)-8-[3-[3-(hydroxymethyl)azetidin-1-yl]sulfonylphenyl]-N-propyl-3H-1-benzazepine-4-carboxamide, Bz-11a (0.1 g, 190.24 μmol, 1 eq) in DMF (2 mL) was added ethyl carbonochloridate (ethylchloroformate) (61.94 mg, 570.72 μmol, 54.33 μL, 3 eq) in one portion at 15° C. The mixture was stirred at 15° C. for 1 hour. LCMS and HPLC showed the desired was detected. The mixture was filtered and purified by prep-HPLC (column: Waters Xbridge BEH C18 100×25 mm, Sum; mobile phase: [water(0.1% TFA)-ACN]; B %: 25%-45%, 20 min) to give ethyl N-[3-[[2-amino-8-[3-[3-(hydroxymethyl)azetidin-1-yl]sulfonylphenyl]-3H-1-benzazepine-4-carbonyl]-propyl-amino]propyl]carbamate, Bz-13 (0.018 g, 30.11 μmol, 15.83% yield) as light yellow solid. ¹H NMR (CD₃OD, 400 MHz) δ 8.11 (s, 1H), 8.08 (d, J=8.0 Hz, 1H), 7.91 (d, J=8.0 Hz, 1H), 7.83 (d, J=8.0 Hz, 1H), 7.81-7.75 (m, 1H), 7.74-7.68 (m, 2H), 7.12 (s, 1H), 4.07 (brs, 2H), 3.87 (t, J=8.0 Hz, 2H), 3.61 (m, 2H), 3.55 (m, 2H), 3.48 (m, 2H), 3.42 (d, J=6.4 Hz, 2H), 3.37 (s, 2H), 3.14 (m, 2H), 2.67-2.51 (m, 1H), 1.93-1.80 (m, 2H), 1.77-1.64 (m, 2H), 1.33-1.06 (m, 3H), 0.95 (s, 3H).

### Example 10 Synthesis of Bz-14

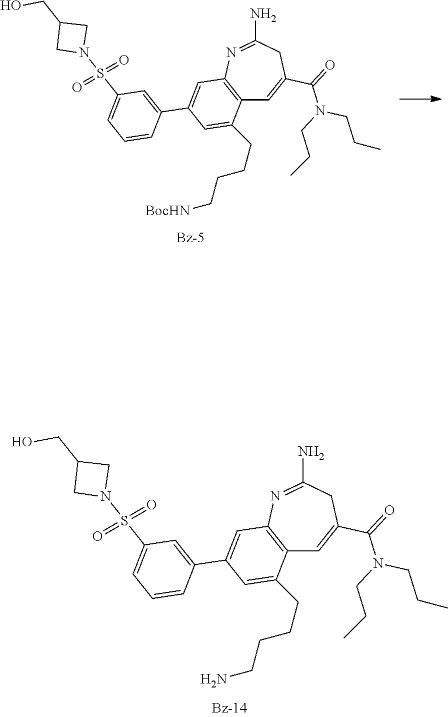 [SMILES: CCCN(CCC)C(=O)C1=Cc2c(CCCCNC(=O)OC(C)(C)C)cc(-c3cccc(S(=O)(=O)N4CC(CO)C4)c3)cc2N=C(N)C1 ; CCCN(CCC)C(=O)C1=Cc2c(CCCCN)cc(-c3cccc(S(=O)(=O)N4CC(CO)C4)c3)cc2N=C(N)C1]

2-Amino-6-(4-aminobutyl)-8-(3-((3-(hydroxymethyl)azetidin-1-yl)sulfonyl)phenyl)-N,N-dipropyl-3H-benzo[b]azepine-4-carboxamide, Bz-14 was synthesized from Bz-5 according to the procedure described for Bz-11a. LC/MS [M+H]582.31 (calculated); LC/MS [M+H]582.57 (observed).

### Example 11 Synthesis of Bz-15

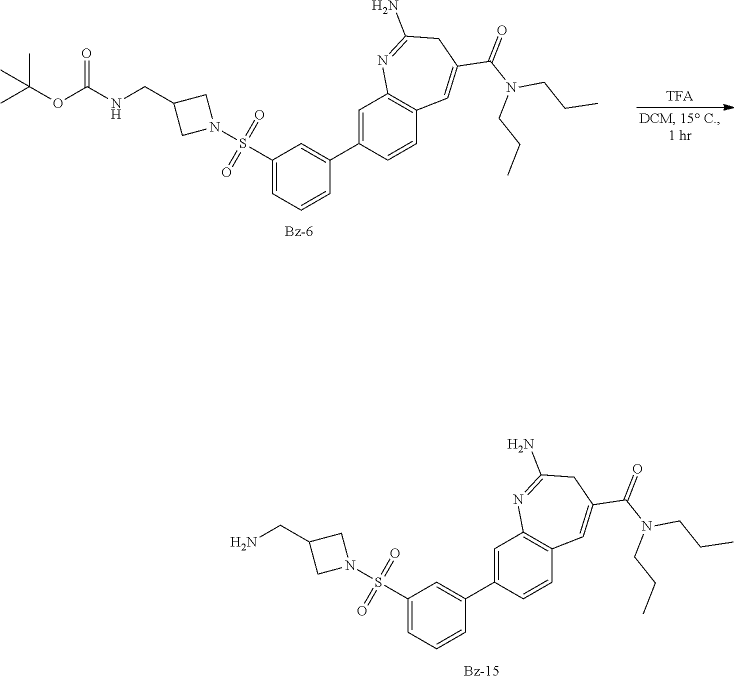 [SMILES: CCCN(CCC)C(=O)C1=Cc2ccc(-c3cccc(S(=O)(=O)N4CC(CNC(=O)OC(C)(C)C)C4)c3)cc2N=C(N)C1 ; CCCN(CCC)C(=O)C1=Cc2ccc(-c3cccc(S(=O)(=O)N4CC(CN)C4)c3)cc2N=C(N)C1]

To a solution of tert-butyl N-[[1-[3-[2-amino-4-(dipropylcarbamoyl)-3H-1-benzazepin-8-yl]phenyl]sulfonylazetidin-3-yl]methyl]carbamate, Bz-6 (0.15 g, 245.99 μmol, 1 eq) in DCM (20 mL) was added TFA (56.10 mg, 491.98 μmol, 36.43 μL, 2 eq) at 25° C. and stirred for 1 hour. The mixture was concentrated in reduced pressure at 40° C. The residue was purified by prep-HPLC (column: Nano-micro Kromasil C18 100×30 mm Sum; mobile phase: [water (0.1% TFA)-ACN]; B %: 25%-50%, 10 min) to give 2-amino-8-[3-[3-(aminomethyl)azetidin-1-yl]sulfonylphenyl]-N,N-dipropyl-3H-1-benzazepine-4-carboxamide, Bz-15 (0.0546 g, 105.69 μmol, 42.97% yield, 98.66% purity) as a yellow solid. ¹H NMR (MeOD-d₄, 400 MHz) δ 8.16-8.07 (m, 2H), 7.92 (d, J=8.0 Hz, 1H), 7.83 (t, J=7.6 Hz, 1H), 7.79-7.72 (m, 2H), 7.68 (d, J=8.4 Hz, 1H), 7.09 (s, 1H), 3.96 (t, J=8.4 Hz, 2H), 3.67-3.63 (m, 2H), 3.50-3.42 (m, 4H), 3.37 (s, 2H), 3.05 (d, J=7.4 Hz, 2H), 2.78-2.65 (m, 1H), 1.75-1.66 (m, 4H), 1.08-0.82 (m, 6H). LC/MS [M+H]510.25 (calculated); LC/MS [M+H]510.10 (observed). Example 12 Synthesis of Bz-16

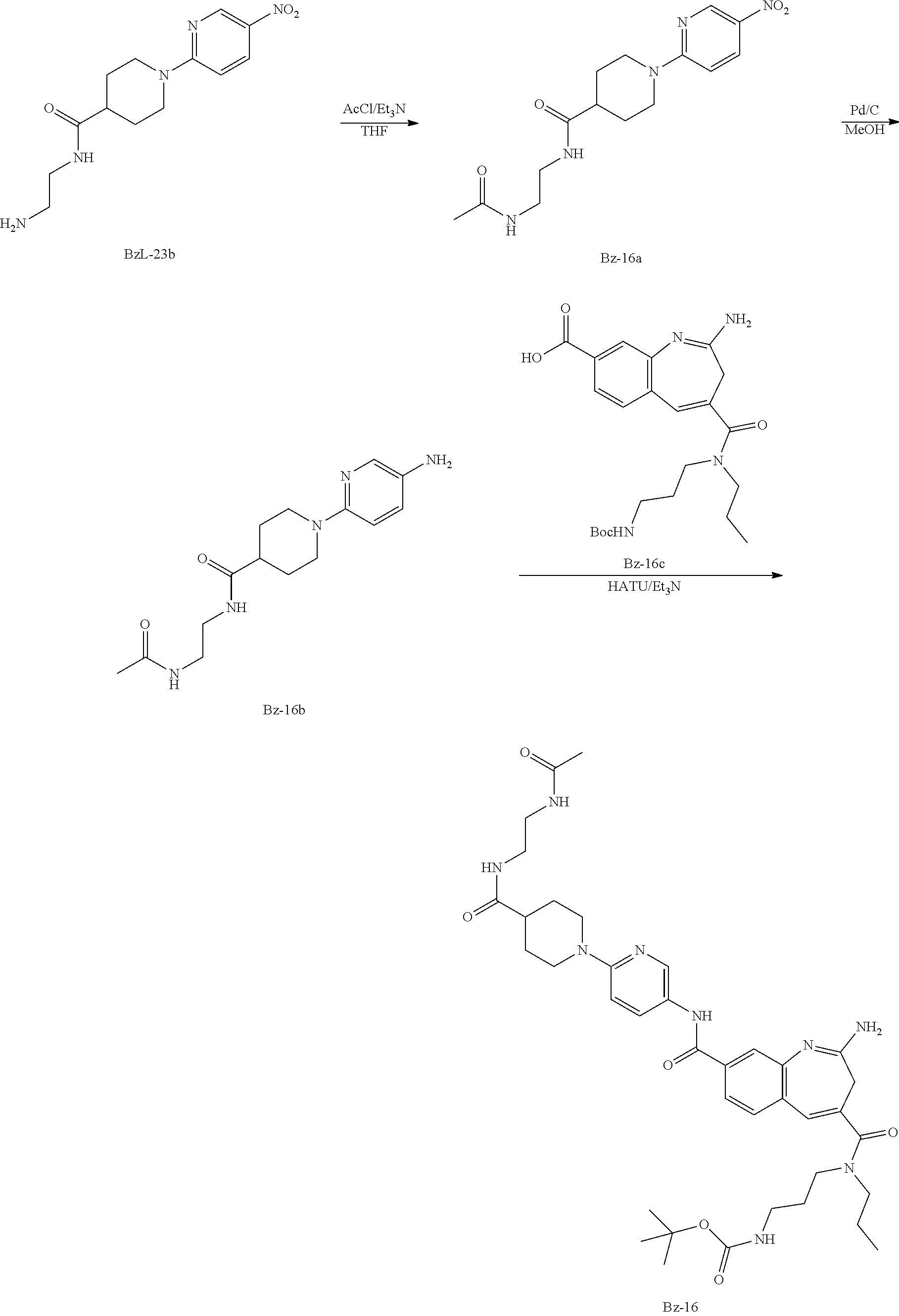

### Synthesis of N-(2-acetamidoethyl)-1-(5-nitropyridin-2-yl) piperidine-4-carboxamide, Bz-16a

To a mixture of acetyl chloride (142.82 mg, 1.82 mmol, 129.83 μL, 3 eq) and N-(2-aminoethyl)-1-(5-nitro-2-pyridyl)piperidine-4-carboxamide, BzL-23b (0.2 g, 606.46 μmol, 1 eq, HCl) in THE (10 mL) was added Et₃N (245.47 mg, 2.43 mmol, 337.65 μL, 4 eq) at 25° C. under N₂. The mixture was stirred at 25° C. for 1 hour. LCMS showed the reaction was completed. The mixture was pour into water (20 mL). The mixture was filtered to give Bz-16a (0.2 g, 596.38 μmol, 98.34% yield) as a yellow solid. ¹H NMR (DMSO-d₆, 400 MHz) δ 8.95 (d, J=2.4 Hz, 1H), 8.19 (dd, J=9.6, 2.4 Hz, 1H), 7.78-7.98 (m, 2H), 6.95 (d, J=9.6 Hz, 1H), 4.50 (d, J=9.6 Hz, 2H), 2.93-3.15 (m, 7H), 1.73-1.80 (m, 5H), 1.43-1.62 (m, 2H), 1.07-1.28 (m, 3H).

### Synthesis of N-(2-acetamidoethyl)-1-(5-aminopyridin-2-yl) piperidine-4-carboxamide, Bz-16b

To a solution of N-(2-acetamidoethyl)-1-(5-nitro-2-pyridyl)piperidine-4-carboxamide, Bz-16a (0.2, 596.38 μmol, 1 eq) in MeOH (20 mL) was added Pd/C (0.2 g, 5% purity) under N₂. The suspension was degassed under vacuum and purged with H₂ several times. The mixture was stirred under H₂ (15 psi) at 25° C. for 4 hours. LCMS showed the reaction was completed. The mixture was filtered and concentrated to give Bz-16b (0.18 g, 589.44 μmol, 98.84% yield) as yellow solid.

### Synthesis of tert-butyl (3-(8-((6-(4-((2-acetamidoethyl)carbamoyl)piperidin-1-yl)pyridin-3-yl)carbamoyl)-2-amino-N-propyl-3H-benzo[b]azepine-4-carboxamido)propyl)carbamate, Bz-16

To a mixture of 2-amino-4-[3-(tert-butoxycarbonylamino) propyl-propyl-carbamoyl]-3H-1-benzazepine-8-carboxylic acid, Bz-16c (0.22 g, 494.91 μmol, 1 eq) HATU (225.82 mg, 593.90 μmol, 1.2 eq) in DMF (5 mL) was added Et₃N (150.24 mg, 1.48 mmol, 206.66 μL, 3 eq) at 25° C. The mixture was stirred at 25° C. for 5 min, then N-(2-acetamidoethyl)-1-(5-amino-2-pyridyl)piperidine-4-carboxamide, Bz-16b (151.13 mg, 494.91 μmol, 1 eq) was added to the mixture, stirred for 30 min. The mixture was poured into water (50 mL). The aqueous phase was extracted with ethyl acetate (50 mL). The combined organic phase was washed with brine (50 mL), dried with anhydrous Na₂SO₄, filtered and concentrated in vacuum. The residue was purified by prep-HPLC column: Welch Xtimate C18 150×25 mm, Sum; mobile phase: [water(10 mM NH₄HCO₃)-ACN]; B %: 30%-50%, 10.5 min to afford Bz-16 (96 mg, 131.17 μmol, 26.50% yield) as an off-white solid. ¹H NMR (MeOD, 400 MHz) δ 8.39 (d, J=2.6 Hz, 1H), 7.90 (dd, J=9.2, 2.6 Hz, 1H), 7.69 (d, J=1.2 Hz, 1H), 7.54-7.60 (m, 1H), 7.46 (br d, J=8.0 Hz, 1H), 6.85-6.95 (m, 2H), 4.30 (d, J=13.6 Hz, 2H), 3.39-3.53 (m, 4H), 3.28 (s, 2H), 3.08-3.12 (m, 2H), 2.83-2.93 (m, 2H), 2.37-2.47 (m, 1H), 1.94 (s, 3H), 1.60-1.90 (m, 8H), 1.24-1.50 (m, 9H). LC/MS [M+H]732.42 (calculated); LC/MS [M+H]732.40 (observed).

### Example 13 Synthesis of Bz-17

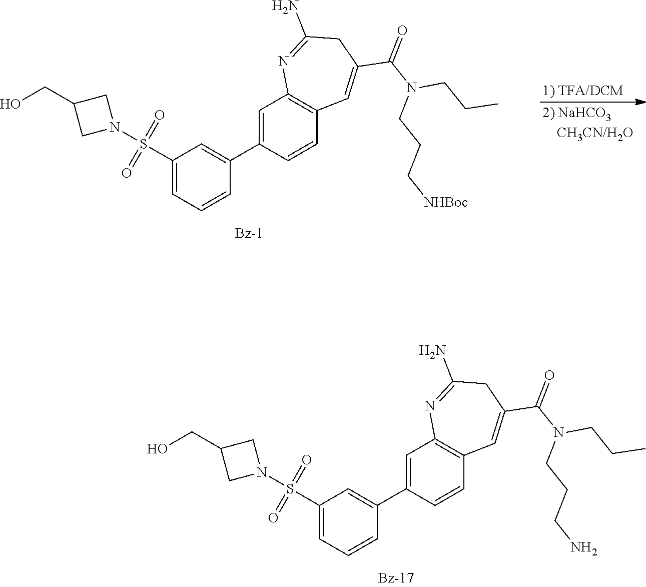 [SMILES: CCCN(CCCNC(=O)OC(C)(C)C)C(=O)C1=Cc2ccc(-c3cccc(S(=O)(=O)N4CC(CO)C4)c3)cc2N=C(N)C1 ; CCCN(CCCN)C(=O)C1=Cc2ccc(-c3cccc(S(=O)(=O)N4CC(CO)C4)c3)cc2N=C(N)C1]

To a solution of tert-butyl N-[3-[[2-amino-8-[3-[3-(hydroxymethyl)azetidin-1-yl]sulfonylphenyl]-3H-1-benzazepine-4-carbonyl]-propyl-amino]propyl]carbamate, Bz-1 (1.5 g, 2.40 mmol, 1 eq) in DCM (20 mL) was added TFA (6.16 g, 54.03 mmol, 4 mL, 22.54 eq) at 25° C. under N₂ and then stirred at this temperature for 1 h. The reaction mixture was concentrated under reduced pressure. The residue was diluted with CH₃CN (30 mL) and H₂O (10 mL) and adjusted pH=8-9 with aq. NaHCO₃ at 0° C. The mixture was stirred for 30 min at 25° C. and then concentrated under reduced pressure to remove CH₃CN. The aqueous phase was extracted with DCM/i-PrOH=3/1 (20 mL×3), dried over Na₂SO₄, filtered and concentrated under reduced pressure. The residue was purified by prep-HPLC (TFA condition; column: Luna® (Phenomenex) C18 250*80 mm*10 μm (micron); mobile phase: [water(0.1% TFA)-ACN]; B %: 10%-40%, 20 min) to afford 2-amino-N-(3-aminopropyl)-8-[3-[3-(hydroxymethyl)azetidin-1-yl]sulfonylphenyl]-N-propyl-3H-1-benzazepine-4-carboxamide, Bz-17 (1.00 g, 1.57 mmol, 65.48% yield, TFA salt) as a white solid. ¹H NMR (MeOD-d₄, 400 MHz) δ8.14-8.05 (m, 2H), 7.91 (d, J=7.6 Hz, 1H), 7.86-7.81 (m, 1H), 7.80-7.72 (m, 2H), 7.71-7.67 (m, 1H), 7.15 (s, 1H), 3.87 (t, J=8.0 Hz, 2H), 3.65-3.57 (m, 4H), 3.55-3.52 (m, 2H), 3.45-3.36 (m, 4H), 3.04-3.01 (m, 2H), 2.63-2.53 (m, 1H), 2.04 (quin, J=7.2 Hz, 2H), 1.77-1.70 (m, 2H), 0.94 (br t, J=6.8 Hz, 3H). LC/MS [M+H]526.2 (calculated); LC/MS [M+H]526.2 (observed).

### Example 14 Synthesis of Bz-18

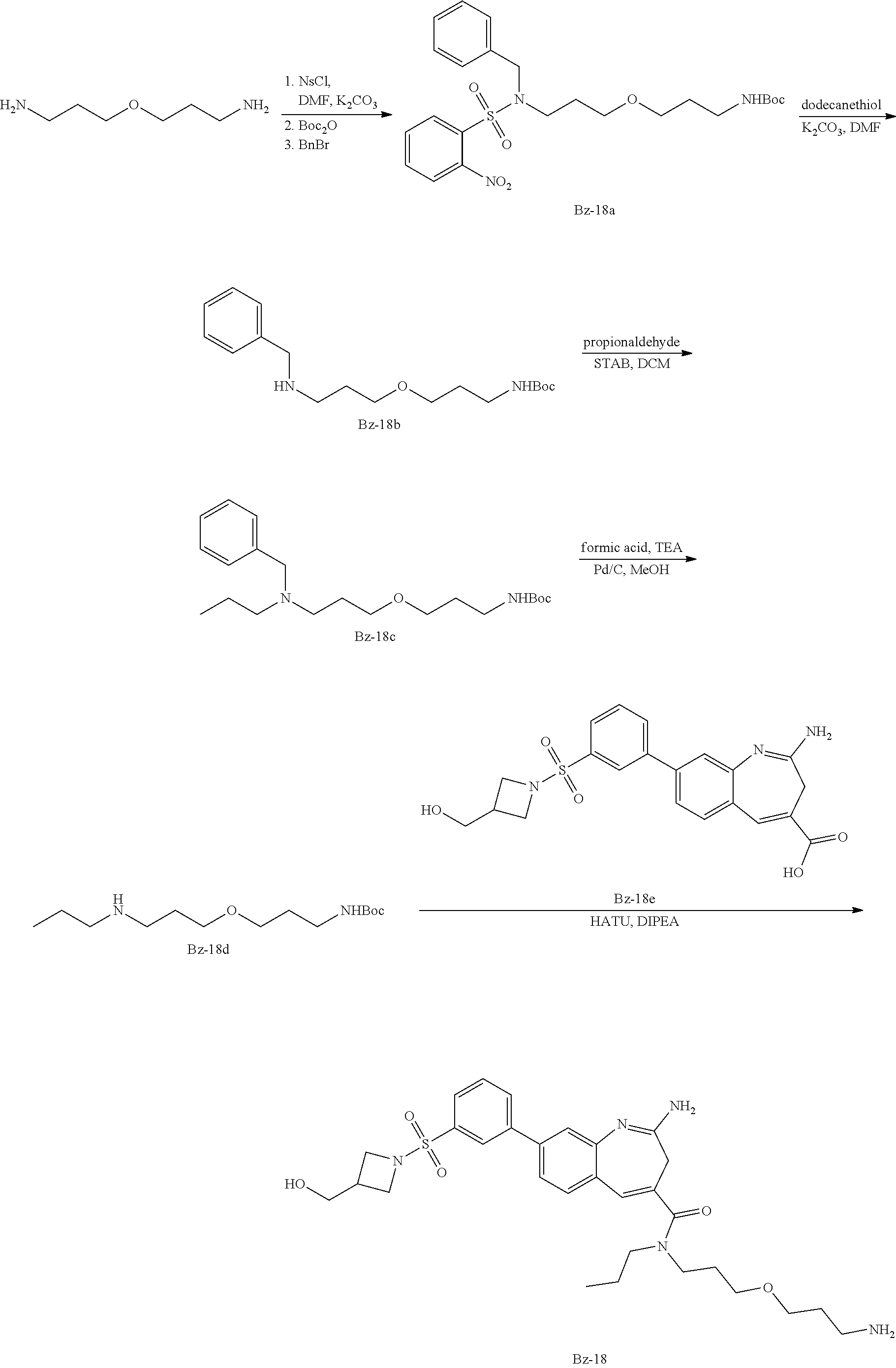

### Preparation of tert-butyl (3-(3-((N-benzyl-2-nitrophenyl)sulfonamido)propoxy)propyl)carbamate, Bz-18a

3,3′-Oxybis(propan-1-amine) (0.5 g, 3.8 mmol, 1 eq.) and potassium carbonate (1.3 g, 9.5 mmol, 2.5 eq.) were taken up in 10 ml DMF. 2-Nitrophenyl sulfonyl chloride (0.84 g, 3.8 mmol, 1 eq.) was added and the reaction monitored by LCMS. Di-tert-butyl dicarbonate (0.87 ml, 3.8 mmol, 1 eq.) was subsequently added. After approximately one additional hour, benzyl bromide (0.45 ml, 3.8 mmol, 1 eq.) was added and the reaction heated to 75° C. Upon completion, the reaction was filtered, concentrated, and purified by flash chromatography to give Bz-18a (0.47 g, 0.93 mmol, 25%). LC/MS [M+H]508.21 (calculated); LC/MS [M+H]508.43 (observed).

### Preparation of tert-butyl (3-(3-(benzylamino)propoxy)propyl)carbamate, Bz-18b

Bz-18a (0.47 g, 0.93 mmol, 1 eq.) was dissolved in DMF. Potassium carbonate (0.19 g, 1.4 mmol, 1.5 eq.) was added, followed by dodecanethiol (0.33 ml, 1.4 mmol, 1.5 eq.). The reaction was stirred at 60° C. overnight, and then purified by column chromatography to give Bz-18b (0.18 g, 0.57 mmol, 61%). LC/MS [M+H]323.23 (calculated); LC/MS [M+H]323.38 (observed).

### Preparation of tert-butyl (3-(3-(benzyl(propyl)amino)propoxy)propyl)carbamate, Bz-18c

Bz-18b (0.183 g, 0.57 mmol, 1 eq.) was dissolved in DCM. Propionaldehyde (0.1 ml, 1.4 mmol, 2.5 eq.) and sodium triacetoxyborohydride (0.3 g, 1.4 mmol, 2.5 eq.) were added. The reaction was stirred at room temperature, then concentrated and purified by HPLC to give Bz-18c (0.058 g, 0.159 mmol, 31%). LC/MS [M+H]365.28 (calculated); LC/MS [M+H]365.44 (observed).

### Preparation of tert-butyl (3-(3-(propylamino)propoxy)propyl)carbamate, Bz-18d

Bz-18c (0.058 g, 0.159 mmol, 1 eq.) was dissolved in 4 ml methanol. To the solution were added triethylamine (0.067 ml, 0.48 mmol, 3 eq.), followed by formic acid (0.015 ml, 0.40 mmol, 2.5 eq.) and then Pd/C (5 mg, 10 wt %). The mixture was heated to 60° C. Upon consumption of starting material, the reaction mixture was filtered and concentrated to give Bz-18d (0.007 g, 0.0092 mmol, 26%). LC/MS [M+H]275.23 (calculated); LC/MS [M+H]275.27 (observed).

### Preparation of Bz-18

2-Amino-8-(3-((3-(hydroxymethyl)azetidin-1-yl)sulfonyl)phenyl)-3H-benzo[b]azepine-4-carboxylic acid, Bz-18e (0.025 g, 0.075 mmol, 1 eq.), Bz-18d (0.02 g, 0.075 mmol, 1 eq.), and diisopropylethylamine (0.065 ml, 0.38 mmol, 5 eq.) were dissolved in DMF. HATU (0.043 g, 0.113 mmol, 1.5 eq.) was added and the mixture stirred at room temperature. When complete, the reaction mixture was concentrated and purified by RP-HPLC. The isolated product was concentrated, dissolved in minimal TFA, and allowed to stand at room temperature for 15 minutes. The solution was then concentrated, purified by RP-HPLC, and lyophilized to give 2-amino-N-(3-(3-aminopropoxy)propyl)-8-(3-((3-(hydroxymethyl)azetidin-1-yl)sulfonyl)phenyl)-N-propyl-3H-benzo[b]azepine-4-carboxamide, Bz-18 as a white powder (1.2 mg, 0.002 mmol, 3%). LC/MS [M+H]584.29 (calculated); LC/MS [M+H]584.50 (observed).

### Example 15 Synthesis of Bz-19

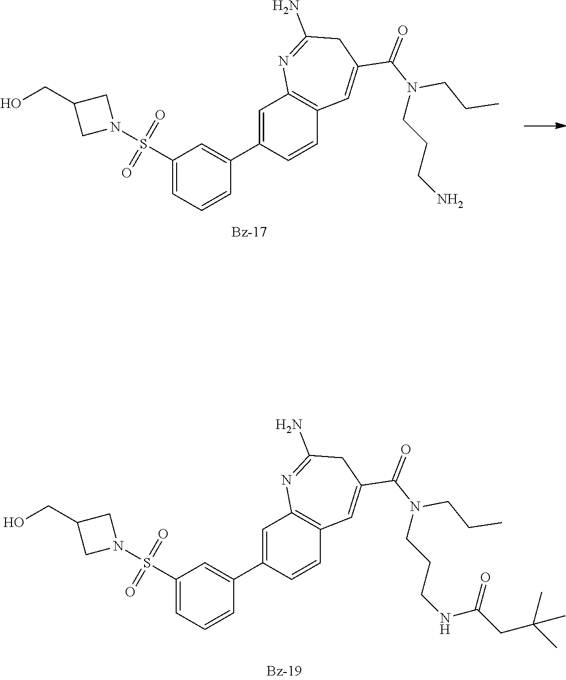 [SMILES: CCCN(CCCN)C(=O)C1=Cc2ccc(-c3cccc(S(=O)(=O)N4CC(CO)C4)c3)cc2N=C(N)C1 ; CCCN(CCCNC(=O)CC(C)(C)C)C(=O)C1=Cc2ccc(-c3cccc(S(=O)(=O)N4CC(CO)C4)c3)cc2N=C(N)C1]

A vial was charged with Bz-17 (0.0275 mmol), diisopropylethylamine (15 μL, 0.0825 mmol), tert-butylacetyl chloride (0.0275 mmol), 250 μL DCM, and 250 μL DMF. The reaction was maintained for three hours and purified by normal phase chromatography using a 0-10% MeOH:DCM gradient affording 6.6 mg of 2-amino-N-(3-(3,3-dimethylbutanamido)propyl)-8-(3-((3-(hydroxymethyl)azetidin-1-yl)sulfonyl)phenyl)-N-propyl-3H-benzo[b]azepine-4-carboxamide, Bz-19 in 39% yield. LC/MS [M+H]624.3 (calculated); LC/MS [M+H]624.3 (observed).

### Example 16 Synthesis of Bz-20

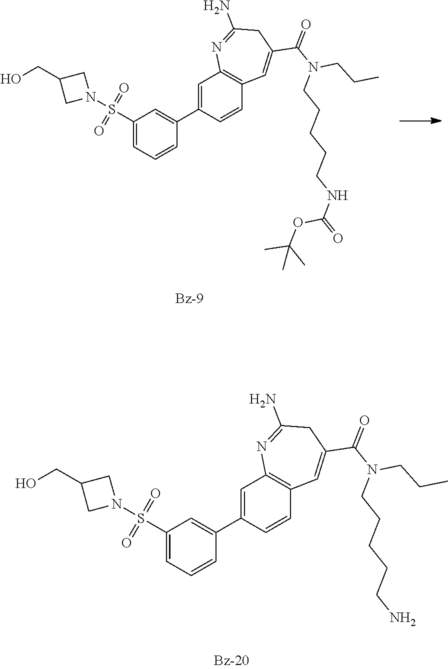 [SMILES: CCCN(CCCCCNC(=O)OC(C)(C)C)C(=O)C1=Cc2ccc(-c3cccc(S(=O)(=O)N4CC(CO)C4)c3)cc2N=C(N)C1 ; CCCN(CCCCCN)C(=O)C1=Cc2ccc(-c3cccc(S(=O)(=O)N4CC(CO)C4)c3)cc2N=C(N)C1]

A vial was charged with Bz-9 (28 mg, 0.043 mmol), 300 μL DCM and 100 μL trifluoroacetic acid. The reaction was maintained for 1 h, upon which it was concentrated under reduced pressure. The resultant oil was azeotroped thrice with 1 mL toluene, after which was added 1 mL methanol and K₂CO₃ (38 mg, 0.28 mmol). After stirring for 16 h, the reaction was filtered and concentrated under reduced pressure and then purified by reverse phase preparative HPLC utilizing a 25-75% gradient of acetonitrile:water containing 0.1% trifluoroacetic acid. The purified fractions were combined and lyophilized to afford 5.8 mg of 2-amino-N-(5-aminopentyl)-8-(3-((3-(hydroxymethyl)azetidin-1-yl)sulfonyl)phenyl)-N-propyl-3H-benzo[b]azepine-4-carboxamide, Bz-20 in 24% yield. LC/MS [M+H]554.28 (calculated); LC/MS [M+H]554.47 (observed).

### Example 17 Synthesis of Bz-21

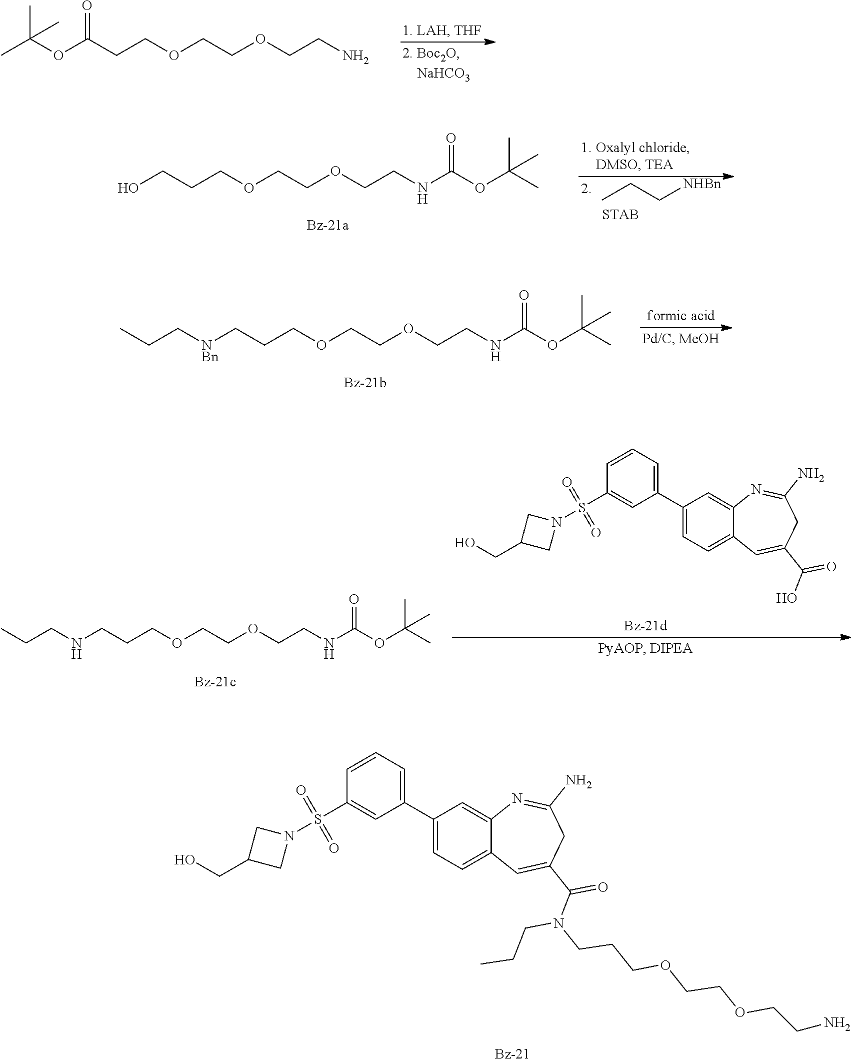

Preparation of tert-butyl (2-(2-(3-hydroxypropoxy)ethoxy)ethyl)carbamate, Bz-21a tert-butyl 3-(2-(2-aminoethoxy)ethoxy)propanoate (0.5 g, 2.1 mmol, 1 eq.) was dissolved in THF. Lithium aluminum hydride (0.244 g, 6.4 mmol, 3 eq.) was added, and the reaction heated to 60° C. Upon complete ester reduction, the reaction was cooled on ice and saturated aqueous sodium bicarbonate was added. The mixture was stirred for 10 minutes, and then Di-tert-butyl dicarbonate (0.49 ml, 2.1 mmol, 1 eq.) added. The reaction was stirred at room temperature, and then concentrated to remove THE before HPLC purification to give Bz-21a (0.205 g, 0.78 mmol, 36%). LC/MS [M+H]264.18 (calculated); LC/MS [M+H]264.27 (observed).

### Preparation of tert-butyl (2-(2-(3-(benzyl(propyl)amino)propoxy)ethoxy)ethyl)carbamate, Bz-21b

Oxalyl chloride (0.205 ml, 2.4 mmol, 3 eq.) was dissolved in 0.5 ml DCM at −78° C. DMSO (0.34 ml, 4.8 mmol, 6 eq.) was added dropwise. The reaction was stirred at −78° C. for 15 minutes, then Bz-21a (0.21 g, 0.80 mmol, 1 eq.) added dropwise as a solution in 0.5 ml DCM. The reaction was stirred 30 minutes at −78° C., and then triethylamine (1 ml, 7.2 mmol, 9 eq.) was added dropwise. The reaction was stirred 30 more minutes at −78° C., then removed from cooling and allowed to warm to ambient temperature over 30 minutes. N-Benzylpropan-1-amine (0.119 g, 0.80 mmol, 1 eq.) and sodium triacetoxyborohydride, STAB (0.845 g, 4.0 mmol, 5 eq.) were suspended in 2 ml DCM. The crude aldehyde solution was added to the stirring amine solution. After 30 minutes, the reaction was added to a separatory funnel and washed with saturated NaHCO₃, water, and then brine. The organic fraction was dried over sodium sulfate, filtered, concentrated, and then purified by RP-HPLC to give Bz-21b (0.228 g, 0.58 mmol, 73%). LC/MS [M+H]395.29 (calculated); LC/MS [M+H]395.44 (observed).

### Preparation of tert-butyl (2-(2-(3-(propylamino)propoxy)ethoxy)ethyl)carbamate, Bz-21c

Bz-21b (0.228 g, 0.58 mmol, 1 eq.) was dissolved in methanol. Formic acid (0.033 mol, 0.87 mmol, 1.5 eq.) was added, followed by 10 wt % Pd/C (0.02 g). The reaction was stirred at 60° C. and then filtered, concentrated, and purified by HPLC to give Bz-21c as a TFA salt (0.193 g, 0.46 mmol, 80%). LC/MS [M+H]305.24 (calculated); LC/MS [M+H]305.38 (observed).

Preparation of Bz-21: 2-Amino-8-(3-((3-(hydroxymethyl)azetidin-1-yl)sulfonyl)phenyl)-3H-benzo[b]azepine-4-carboxylic acid, Bz-21d (0.042 g, 0.099 mmol, 1 eq.), Bz-21c (0.03 g, 0.099 mmol, 1 eq.), and diisopropylethylamine (0.1 ml, 0.57 mmol, 5.8 eq.) were dissolved in DMF. 7-Aza-benzotriazol-1-yloxy-tripyrrolidino-phosphonium hexafluorophosphate, PyAOP, CAS Reg. No. 156311-83-0 (0.077 g, 0.15 mmol, 1.5 eq.) was added and the mixture stirred at room temperature. When complete, the reaction mixture was concentrated and purified by HPLC. The isolated product was concentrated, dissolved in minimal TFA, and allowed to stand at room temperature for 15 minutes. The solution was then concentrated and purified by HPLC to give an oil that was triturated with diethyl ether to give 2-amino-N-(3-(2-(2-aminoethoxy)ethoxy)propyl)-8-(3-((3-(hydroxymethyl)azetidin-1-yl)sulfonyl)phenyl)-N-propyl-3H-benzo[b]azepine-4-carboxamide, Bz-21 as a white solid (0.037 g, 0.060 mmol, 61%). LC/MS [M+H]614.30 (calculated); LC/MS [M+H]614.58 (observed).

### Example 18 Synthesis of Bz-22

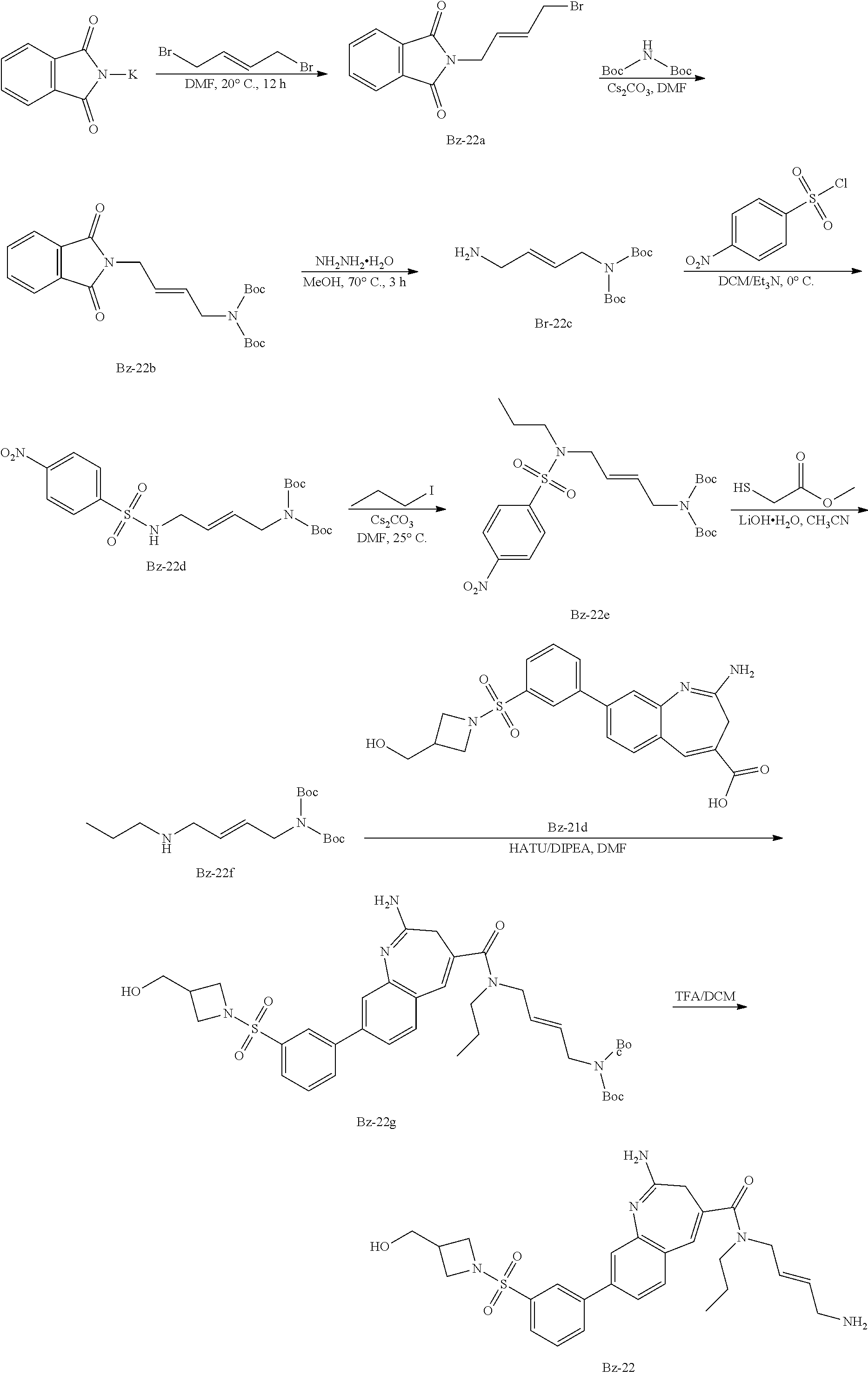

### Preparation of (E)-2-(4-bromobut-2-en-1-yl)isoindoline-1,3-dione, Bz-22a

To a solution of (1,3-dioxoisoindolin-2-yl)potassium (7.5 g, 40.5 mmol, 1 eq) in DMF (100 mL) was added (E)-1,4-dibromobut-2-ene (17.3 g, 80.9 mmol, 2 eq). The mixture was stirred at 20° C. for 12 h and then diluted with water (200 mL) and extracted with EtOAc (80 mL×3). The organic layer was washed with brine (50 mL), dried over Na₂SO₄, filtered and concentrated. The residue was purified by flash silica gel chromatography (ISCO®; 12 g SepaFlash® Silica Flash Column, Eluent of 0˜60% Ethyl acetate/Petroleum ether gradient at 60 mL/min) to give Bz-22a (8.6 g, 30.7 mmol, 75.82% yield) as white solid. ¹H NMR (CDCl₃, 400 MHz) δ7.90-7.83 (m, 2H), 7.78-7.70 (m, 2H), 6.01-5.90 (m, 1H), 5.89-5.79 (m, 1H), 4.32 (d, J=5.6 Hz, 2H), 3.92 (d, J=7.2 Hz, 2H).

### Preparation of tert-butyl N-tert-butoxycarbonyl-N-[(E)-4-(1,3-dioxoisoindolin-2-yl)but-2-enyl]carbamate, Bz-22b

To a solution of Bz-22a (11 g, 39.3 mmol, 1 eq) in DMF (200 mL) was added Cs₂CO₃ (19.2 g, 58.9 mmol, 1.5 eq) and tert-butyl N-tert-butoxycarbonylcarbamate (11.1 g, 51. mmol, 1.3 eq). The mixture was stirred at 20° C. for 12 h and then diluted with water (400 mL) and extracted with EtOAc (100 mL×3). The organic layer was washed with brine (80 mL×3), dried over Na₂SO₄, filtered and concentrated. The residue was purified by flash silica gel chromatography (ISCO®; 5 g SepaFlash® Silica Flash Column, Eluent of 0˜70% Ethyl acetate/Petroleum ether gradient @65 mL/min) to give Bz-22b (16 g, 38.4 mmol, 97.83% yield) as white solid. ¹H NMR (DMSO-d₆, 400 MHz) δ7.90-7.83 (m, 4H), 5.63-5.53 (m, 2H), 4.20-4.12 (m, 2H), 4.05-3.99 (m, 2H), 1.36 (s, 18H)

### Preparation of tert-butyl N-[(E)-4-aminobut-2-enyl]-N-tert-butoxycarbonyl-carbamate, Bz-22c

To a solution of Bz-22b (18 g, 43.2 mmol, 1 eq) in MeOH (200 mL) was added hydrazine; hydrate (10.2 g, 173 mmol, 9.90 mL 85% purity, 4 eq) at 20° C. and then stirred at 70° C. for 3 h. The mixture was filtered and the filtrate was concentrated. The crude product was triturated with CH₃CN at 20° C. for 20 min and filtered, the filtrate was concentrated to give Bz-22c (10 g, 34.9 mmol, 80.80% yield) as light yellow oil. ¹H NMR (CDCl₃, 400 MHz) δ5.78-5.69 (m, 1H), 5.64-5.54 (m, 1H), 4.17-4.09 (m, 2H), 3.31-3.23 (m, 2H), 1.49 (s, 18H)

### Preparation of tert-butyl N-tert-butoxycarbonyl-N-[(E)-4-[(4-nitrophenyl)sulfonylamino]but-2-enyl]carbamate, Bz-22d

To a solution of Bz-22c (1 g, 3.49 mmol, 1 eq) in DCM (10 mL) was added TEA (706.72 mg, 6.98 mmol, 972.10 uL (microliters), 2 eq) and 4-nitrobenzenesulfonyl chloride (851.29 mg, 3.84 mmol, 1.1 eq) at 0° C. under N₂. The mixture was stirred at 25° C. for 1 h and then quenched by addition of H₂O (20 mL) at 0° C., and then extracted with EtOAc (10 mL×3). The combined organic layers were washed with brine (5 mL), dried over Na₂SO₄, filtered and concentrated under reduced pressure to give a residue which was purified by column chromatography (SiO₂, Petroleum ether/Ethyl acetate=I/O to 1/1) to give Bz-22d (1.2 g, 2.54 mmol, 72.74% yield) as a light yellow oil. ¹H NMR (CDCl₃, 400 MHz) δ8.41-8.35 (m, 2H), 8.05 (d, J=9.2 Hz, 2H), 5.71-5.61 (m, 1H), 5.57-5.47 (m, 1H), 4.61 (t, J=5.6 Hz, 1H), 4.10 (d, J=5.6 Hz, 2H), 3.67 (t, J=6.0 Hz, 2H), 1.49 (s, 18H).

### Preparation of tert-butyl N-tert-butoxycarbonyl-N-[(E)-4-[(4-nitrophenyl)sulfonyl-propyl-amino]but-2-enyl]carbamate, Bz-22e

To a solution of Bz-22d (1 g, 2.12 mmol, 1 eq) in DMF (10 mL) was added Cs₂CO₃ (1.38 g, 4.24 mmol, 2 eq) and 1-iodopropane (360.52 mg, 2.12 mmol, 207.19 uL, 1 eq) at 25° C. and then stirred at this temperature for 12 h. The reaction mixture was quenched by addition of H₂O (50 mL) at 0° C., and then extracted with EtOAc (30 mL×3). The combined organic layers were washed with brine (10 mL×3), dried over Na₂SO₄, filtered and concentrated under reduced pressure. The residue was purified by column chromatography (SiO₂, Petroleum ether/Ethyl acetate=I/O to 3/1) to give Bz-22e (0.89 g, 1.73 mmol, 81.71% yield) as a light yellow oil. ¹H NMR (CDCl₃, 400 MHz) δ8.36 (d, J=8.8 Hz, 2H), 7.99 (d, J=8.8 Hz, 2H), 5.74-5.60 (m, 1H), 5.51-5.37 (m, 1H), 4.11 (d, J=7.2 Hz, 2H), 3.86 (d, J=6.4 Hz, 2H), 3.16-3.07 (m, 2H), 1.55-1.46 (m, 20H), 0.86 (t, J=7.6 Hz, 3H)

### Preparation of tert-butyl N-tert-butoxycarbonyl-N-[(E)-4-(propylamino)but-2-enyl]carbamate, Bz-22f

To a solution of Bz-22e (0.79 g, 1.54 mmol, 1 eq) in CH₃CN (10 mL) was added LiOH·H₂O (387.25 mg, 9.23 mmol, 6 eq) and methyl 2-sulfanylacetate (490 mg, 4.61 mmol, 419 μL, 3 eq) at 0° C. The resulting mixture was stirred at 25° C. for 12 h and then filtered and concentrated under reduced pressure. The residue was diluted with H₂O (20 mL) at 0° C., and then adjusted pH=2-3 with 1 M HCl and extracted with MTBE (10 mL×3). The pH of water phase was adjusted to ˜10 with aq. K₂CO₃ and extracted with (10 mL×3). The combined organic layers were dried over Na₂SO₄, filtered and concentrated under reduced pressure to give Bz-22f (0.35 g, 1.07 mmol, 69.28% yield) as a colorless oil. ¹H NMR (CDCl₃, 400 MHz) δ5.79-5.58 (m, 2H), 4.15 (d, J=5.2 Hz, 2H), 3.23 (d, J=5.6 Hz, 2H), 2.56 (t, J=6.8 Hz, 2H), 1.56-1.42 (m, 20H), 0.92 (t, J=7.6 Hz, 3H).

### Preparation of tert-butyl N-[(E)-4-[[2-amino-8-[3-[3-(hydroxymethyl)azetidin-1-yl]sulfonylphenyl]-3H-1-benzazepine-4-carbonyl]-propyl-amino]but-2-enyl]-N-tert-butoxycarbonyl-carbamate, Bz-22g

To a mixture of 2-amino-8-(3-((3-(hydroxymethyl)azetidin-1-yl)sulfonyl)phenyl)-3H-benzo[b]azepine-4-carboxylic acid, Bz-21d (0.45 g, 1.05 mmol, 1 eq) in DMF (5 mL) was added HATU (440 mg, 1.16 mmol, 1.1 eq) and DIPEA (408 mg, 3.16 mmol, 550 μL, 3 eq) at 25° C. After 10 min, Bz-22f (345.75 mg, 1.05 mmol, 1 eq) was added to the mixture at 25° C. and then stirred at this temperature for 1 h. The reaction mixture was poured into ice water (30 mL) at 0° C., and extracted with DCM/i-PrOH=3/1 (20 mL×3). The combined organic layers were dried over Na₂SO₄, filtered and concentrated under reduced pressure to give Bz-22g (0.41 g, crude) as a brown solid.

Preparation of Bz-22: To a solution of tert-butyl N-[(E)-4-[[2-amino-8-[3-[3-(hydroxymethyl)azetidin-1-yl]sulfonylphenyl]-3H-1-benzazepine-4-carbonyl]-propyl-amino]but-2-enyl]-N-tert-butoxycarbonyl-carbamate (13 mg, 17.6 umol (micromoles), 1 eq) in DCM (1 mL) was added TFA (154 mg, 1.35 mmol, 0.1 mL, 76.7 eq) at 25° C. and then stirred at this temperature for 1 h. The reaction mixture was concentrated under reduced pressure. The residue was dissolved with CH₃CN (10 mL) and H₂O (1 mL) and adjusted pH=9 with aq. LiOH at 0° C. The mixture was concentrated under reduced pressure. The residue was purified by prep-HPLC (TFA condition; column: Welch Xtimate C18 100*25 mm*3 um; mobile phase: [water(0.1% TFA)-ACN]; B %: 5%-35%, 12 min) to give 2-amino-N-[(E)-4-aminobut-2-enyl]-8-[3-[3-(hydroxymethyl)azetidin-1-yl]sulfonylphenyl]-N-propyl-3H-1-benzazepine-4-carboxamide, Bz-22 (7 mg, 10.74 umol, 60.97% yield, TFA) as a white solid. ¹H NMR (MeOD-d₄, 400 MHz) δ8.15-8.04 (m, 2H), 7.91 (d, J=8.0 Hz, 1H), 7.86-7.72 (m, 3H), 7.68 (d, J=8.0 Hz, 1H), 7.13 (s, 1H), 6.07-5.94 (m, 1H), 5.89-5.77 (m, 1H), 4.21 (br s, 2H), 3.87 (t, J=8.4 Hz, 2H), 3.67-3.56 (m, 4H), 3.48 (br s, 2H), 3.45-3.37 (m, 4H), 2.68-2.50 (m, 1H), 1.77-1.61 (m, 2H), 0.95-0.93 (m, 3H). LC/MS [M+H]538.2 (calculated); LC/MS [M+H]538.3 (observed).

### Example 19 Synthesis of Bz-23

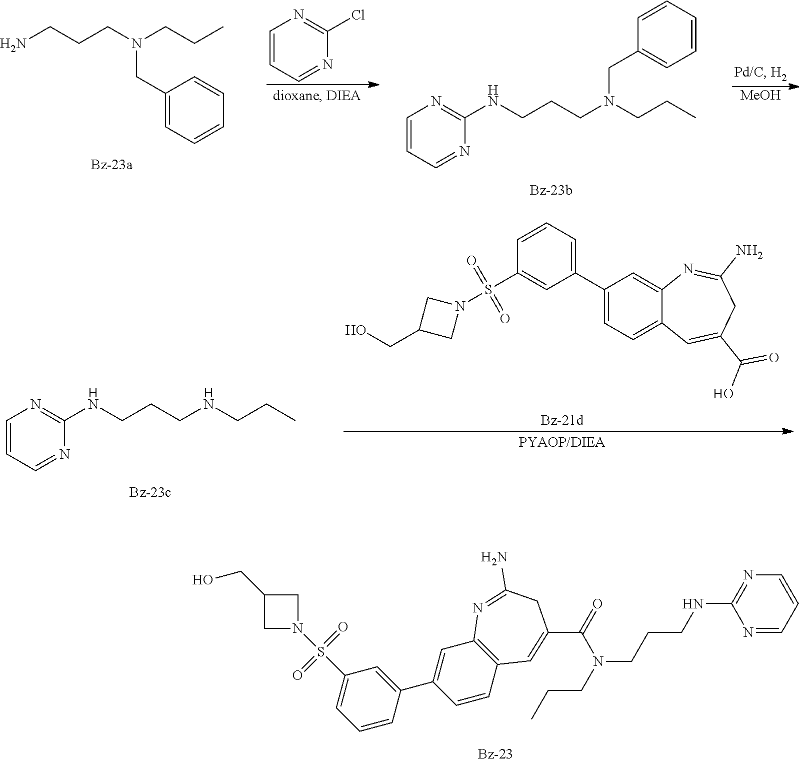

### Preparation of N′-benzyl-N′-propyl-N-pyrimidin-2-yl-propane-1,3-diamine, Bz-23b

A mixture of N′-benzyl-N′-propyl-propane-1,3-diamine, Bz-23a (0.2 g, 823.77 umol, 1 eq, HCl), DIEA (426 mg, 3.30 mmol, 574 μL, 4 eq) in dioxane (4 mL) was stirred at 25° C. for 10 min, and then 2-chloropyrimidine (188.70 mg, 1.65 mmol, 2 eq) was added, then mixture was stirred at 25° C. for 16 h. The reaction was quenched with H₂O (15 mL) and extracted with ethyl acetate (15 mL×3). The combined organic phase was washed with brine (10 mL), dried with anhydrous Na₂SO₄, filtered and concentrated in vacuum. The residue was purified by prep-TLC (SiO₂, DCM:MeOH=7:1) to give Bz-23b (130 mg, 457 umol, 55.49% yield) as yellow oil. ¹H NMR (CDCl₃, 400 MHz) δ8.26 (d, J=4.8 Hz, 2H), 7.38-7.32 (m, 2H), 7.30 (t, J=7.2 Hz, 2H), 7.26-7.20 (m, 1H), 6.49 (t, J=5.2 Hz, 1H), 5.74 (br s, 1H), 3.58 (s, 2H), 3.47-3.39 (m, 2H), 2.54 (t, J=6.8 Hz, 2H), 2.44-2.38 (m, 2H), 1.77 (quin, J=6.4 Hz, 2H), 1.57-1.50 (m, 2H), 0.88 (t, J=7.2 Hz, 3H)

### Preparation of N-propyl-N′-pyrimidin-2-yl-propane-1,3-diamine, Bz-23c

To a solution of Bz-23b (130 mg, 457 umol, 1 eq) in MeOH (10 mL) was added Pd/C (0.1 g, 10% purity) under N₂ atmosphere. The suspension was degassed and purged thrice with hydrogen gas, H₂, the mixture was stirred at 25° C. for 16 h and then filtered and concentrated under reduced pressure. The residue was purified by prep-TLC (SiO₂, DCM:MeOH=5:1) to give Bz-23c (80 mg, 412 umol, 90.09% yield) as a brown oil.

Preparation of Bz-23: To a solution of 2-amino-8-[3-[3-(hydroxymethyl)azetidin-1-yl]sulfonylphenyl]-3H-1-benzazepine-4-carboxylic acid, Bz-21d (264 mg, 618 umol, 1 eq) in DMF (2 mL) was added DIEA (240 mg, 1.85 mmol, 323 μL, 3 eq), 7-Aza-benzotriazol-1-yloxy-tripyrrolidino-phosphonium hexafluorophosphate, PYAOP (483 mg, 927 umol, 1.5 eq) and Bz-23c (120 mg, 618 umol, 1 eq). The mixture was stirred at 25° C. for 1 h, and then filtered and concentrated under reduced pressure. The residue was purified by prep-HPLC WelchXtimateC18100×25 mm×3 um; mobilephase:[water(0.1% TFA)-ACN]; B %:15%-35%, 12 min) to give 2-amino-8-[3-[3-(hydroxymethyl)azetidin-1-yl]sulfonylphenyl]-N-propyl-N-[3-(pyrimidin-2-ylamino)propyl]-3H-1-benzazepine-4-carboxamide, Bz-23 (16 mg, 26.5 umol, 4.29% yield) as a white solid. ¹H NMR (MeOD-d₄, 400 MHz) δ8.38 (br s, 1H), 8.15 (s, 1H), 8.11 (s, 1H), 8.08 (d, J=8.4 Hz, 1H), 7.92 (d, J=8.4 Hz, 1H), 7.85-7.79 (m, 1H), 7.75 (br s, 1H), 7.71 (br s, 1H), 7.53 (s, 1H), 7.11 (br s, 1H), 6.74 (br s, 1H), 3.87 (t, J 8.0 Hz, 2H), 3.62 (dd, J=6.0, 8.0 Hz, 4H), 3.54-3.49 (m, 2H), 3.42 (d, J=6.8 Hz, 2H), 3.35 (br s, 2H), 2.64-2.51 (m, 1H), 2.08-1.95 (m, 2H), 1.77-1.66 (m, 2H), 0.99-0.94 (m, 3H). LC/MS [M+H]604.3 (calculated); LC/MS [M+H]604.3 (observed).

### Example 20 Synthesis of Bz-24

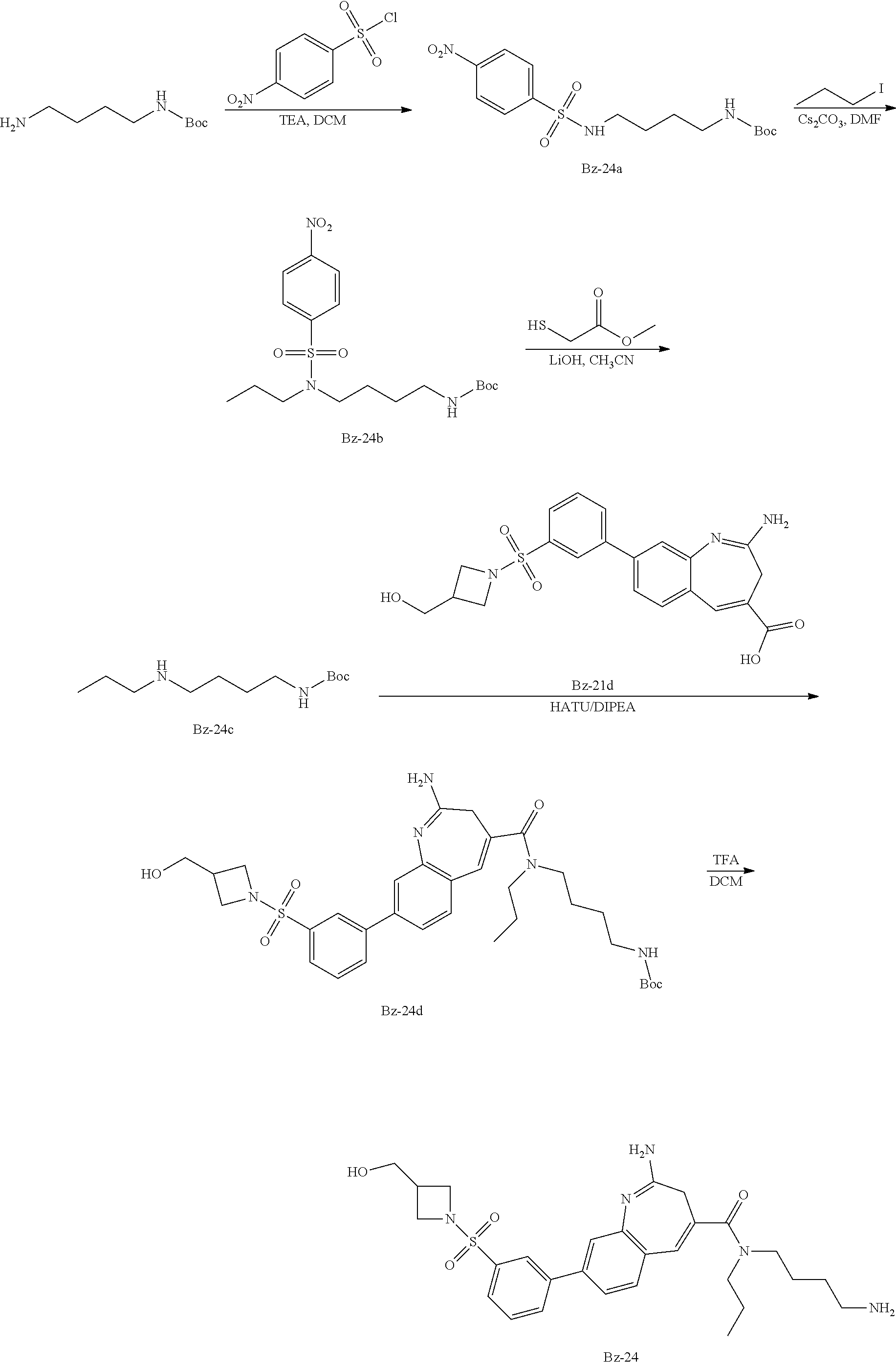

### Preparation of tert-butyl N-[4-[(4-nitrophenyl)sulfonylamino]butyl]carbamate, Bz-24a

To a solution of tert-butyl N-(4-aminobutyl)carbamate (0.5 g, 2.66 mmol, 1 eq) and Et₃N(537 mg, 5.31 mmol, 739 μL, 2 eq) in DCM (5 mL) was added 4-nitrobenzenesulfonyl chloride (647 mg, 2.92 mmol, 1.1 eq) at 0° C. After addition, the resulting mixture was stirred at 25° C. for 1 h and then quenched by addition of H₂O (20 mL) at 0° C., and then extracted with DCM(10 mL×3). The combined organic layers were washed with brine (5 mL), dried over Na₂SO₄, filtered and concentrated under reduced pressure. The residue was triturated with PE/MTBE=10/1 (20 mL) and stirred for 30 min, filtered and the filter cake was dried under reduced pressure to give Bz-24a (0.99 g, 2.65 mmol, 99.82% yield) as a white solid. ¹H NMR (CDCl₃, 400 MHz) δ8.37 (d, J=8.8 Hz, 2H), 8.07 (d, J=8.4 Hz, 2H), 5.28 (br s, 1H), 4.59 (br s, 1H), 3.12-3.03 (m, 4H), 1.58-1.48 (m, 4H), 1.44 (s, 9H)

### Preparation of tert-butyl N-[4-[(4-nitrophenyl)sulfonyl-propyl-amino]butyl]carbamate, Bz-24b

To a solution of Bz-24a (0.99 g, 2.65 mmol, 1 eq) in DMF (7 mL) was added Cs₂CO₃ (1.73 g, 5.30 mmol, 2 eq) and 1-iodopropane (451 mg, 2.65 mmol, 259 μL, 1 eq) at 0° C. The mixture was stirred at 25° C. for 12 h and then poured into ice water (30 mL) at 0° C., and then extracted with EtOAc(20 mL×3). The combined organic layers were washed with brine (10 mL×3), dried over Na₂SO₄, filtered and concentrated under reduced pressure. The residue was triturated with PE/MTBE=10/1 (20 mL) and stirred at 25° C. for 30 min, filtered and the filter cake was dried under reduced pressure to give Bz-24b (0.97 g, 2.33 mmol, 88.06% yield) as a light yellow solid. ¹H NMR (DMSO-d₆, 400 MHz) δ8.39 (d, J=8.8 Hz, 2H), 8.07 (d, J=8.8 Hz, 2H), 6.79 (br t, J=6.0 Hz, 1H), 3.13-3.05 (m, 4H), 2.88 (q, J=6.4 Hz, 2H), 1.54-1.40 (m, 4H), 1.39-1.27 (m, 11H), 0.81 (t, J=7.2 Hz, 3H).

### Preparation of tert-butyl N-[4-(propylamino)butyl]carbamate, Bz-24c

To a solution of Bz-24b (0.97 g, 2.33 mmol, 1 eq) in CH₃CN (10 mL) was added LiOH·H₂O (587.74 mg, 14.01 mmol, 6 eq) and methyl 2-sulfanylacetate (744 mg, 7.00 mmol, 635 μL, 3 eq) at 0° C. The resulting mixture was stirred at 25° C. for 12 h and then filtered and concentrated under reduced pressure. The residue was diluted with H₂O (20 mL) at 0° C., and then adjusted pH=2-3 with 1 M HCl and extracted with MTBE(10 mL×3). The pH of water phase was adjusted to ˜10 with aq. K₂CO₃ and extracted with EtOAc(10 mL×3). The combined organic layers were dried over Na₂SO₄, filtered and concentrated under reduced pressure to give Bz-24c (445 mg, 1.93 mmol, 82.75% yield) as a brown oil. ¹H NMR (DMSO-d₆, 400 MHz) δ6.81 (br s, 1H), 2.89 (q, J=6.4 Hz, 2H), 2.47-2.39 (m, 4H), 1.44-1.31 (m, 15H), 0.85 (t, J=7.6 Hz, 3H).

### Preparation of tert-butyl N-[4-[[2-amino-8-[3-[3-(hydroxymethyl)azetidin-1-yl]sulfonylphenyl]-3H-1-benzazepine-4-carbonyl]-propyl-amino]butyl]carbamate, Bz-24d

To a solution of 2-amino-8-[3-[3-(hydroxymethyl)azetidin-1-yl]sulfonylphenyl]-3H-1-benzazepine-4-carboxylic acid, Bz-21d (100 mg, 234 umol, 1 eq) and DIPEA (90.7 mg, 702 umol, 122.24 uL, 3 eq) in DMF (1 mL) was added HATU (97.8 mg, 257 umol, 1.1 eq) at 25° C. After 10 min, Bz-24c (64.66 mg, 280.72 umol, 1.2 eq) was added at 25° C. and then stirred at this temperature for 1 h. The reaction mixture was filtered and concentrated under reduced pressure. The residue was purified by prep-HPLC (TFA condition; column: Welch Xtimate C18 100*25 mm*3 um; mobile phase: [water(0.1% TFA)-ACN]; B %: 30%-45%, 12 min). Bz-24d (8 mg, 12.50 umol, 5.35% yield) was obtained as a yellow solid. ¹H NMR (MeOD-d₄, 400 MHz) δ8.14-8.04 (m, 2H), 7.92 (d, J=8.0 Hz, 1H), 7.85-7.81 (m, 1H), 7.81-7.76 (m, 1H), 7.73-7.68 (m, 2H), 7.11 (s, 1H), 3.87 (t, J=7.6 Hz, 2H), 3.61 (dd, J=6.0 Hz, 7.6 Hz, 2H), 3.58-3.45 (m, 4H), 3.44-3.35 (m, 4H), 3.12-3.04 (m, 2H), 2.65-2.52 (m, 1H), 1.78-1.63 (m, 4H), 1.55-1.40 (m, 11H), 0.95-0.93 (m, 3H). LC/MS [M+H]640.3 (calculated); LC/MS [M+H]640.3 (observed).

Preparation of Bz-24: To a solution of Bz-24d (0.1 g, 156 umol, 1 eq) in DCM (2 mL) was added TFA (308 mg, 2.70 mmol, 0.2 mL, 17.28 eq) at 25° C. and then stirred at this temperature for 1 h. The reaction mixture was concentrated under reduced pressure. The residue was dissolved with CH₃CN (10 mL) and H₂O (1 mL) and adjusted pH=9 with aq. LiOH at 0° C. The mixture was stirred for 1 h at 25° C. and then filtered and concentrated under reduced pressure. The residue was purified by prep-HPLC (TFA condition; column: Welch Xtimate C18 100*25 mm*3 um; mobile phase: [water(0.1% TFA)-ACN]; B %: 5%-30%, 12 min) to give 2-amino-N-(4-aminobutyl)-8-[3-[3-(hydroxymethyl)azetidin-1-yl]sulfonylphenyl]-N-propyl-3H-1-benzazepine-4-carboxamide, Bz-24 (34 mg, 52.01 umol, 33.28% yield, TFA) as a white solid. ¹H NMR (MeOD-d₄, 400 MHz) δ8.13-8.05 (m, 2H), 7.90 (d, J=8.0 Hz, 1H), 7.85-7.78 (m, 1H), 7.77-7.72 (m, 2H), 7.71-7.65 (m, 1H), 7.10 (s, 1H), 3.86 (t, J=8.4 Hz, 2H), 3.61 (dd, J=5.6 Hz, 7.6 Hz, 2H), 3.58-3.46 (m, 4H), 3.44-3.36 (m, 4H), 3.05-2.94 (m, 2H), 2.64-2.52 (m, 1H), 1.84-1.62 (m, 6H), 1.03-0.85 (m, 3H). LC/MS [M+H]540.3 (calculated); LC/MS [M+H]540.3 (observed).

### Example 21 Synthesis of Bz-25

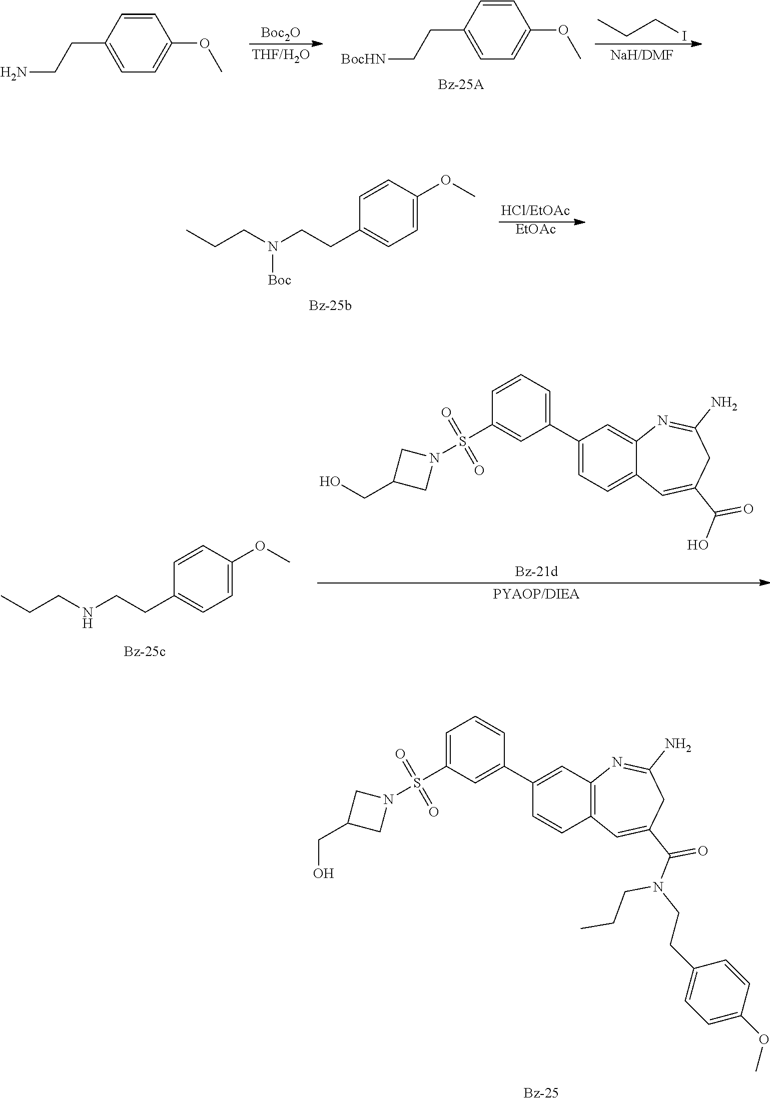

### Preparation of tert-butyl N-[2-(4-methoxyphenyl)ethyl] carbamate, Bz-25a

To a mixture of 2-(4-methoxyphenyl) ethanamine (1 g, 6.61 mmol, 970.87 uL, 1 eq) in THE and H₂O (10 mL) was added Boc₂O (2.17 g, 9.92 mmol, 2.28 mL, 1.5 eq) and then stirred at 25° C. for 30 min under N₂ atmosphere. The mixture was diluted with water and extracted with EtOAc (50 ml×3). The organic layer was washed with brine, dried over Na₂SO₄, filtered and concentrated. The residue was purified by silica gel chromatography (column height:250 mm, diameter:100 mm, 100-200 mesh silica gel, Petroleum ether/Ethylacetate=5/1-1/1) to give Bz-25a (1.60 g, 6.37 mmol, 96.26% yield) as a white solid. ¹H NMR (CDCl₃, 400 MHz) δ7.12 (d, J=8.4 Hz, 2H), 6.85 (d, J=8.4 Hz, 2H), 4.53 (br s, 1H), 3.80 (s, 3H), 3.37-3.33 (m, 2H), 2.74 (br t, J=6.4 Hz, 2H), 1.44 (s, 9H)

### Preparation of tert-butyl 4-methoxyphenethyl(propyl)carbamate, Bz-25b

To a mixture of Bz-25a (0.8 g, 3.18 mmol, 1 eq) and 1-iodopropane (1.08 g, 6.37 mmol, 621 μL, 2 eq) in DMF (8 mL) was added NaH (191 mg, 4.77 mmol, 60% purity, 1.5 eq) at 0° C., and then stirred at 25° C. for 2 hr. The mixture was poured into water (20 mL). The aqueous phase was extracted with ethyl acetate (15 mL×3). The combined organic phase was washed with brine (10 mL), dried with anhydrous Na₂SO₄, filtered and concentrated under reduced pressure to give a residue. The residue was purified by silica gel chromatography (column height:250 mm, diameter: 100 mm, 100-200 mesh silica gel, Petroleum ether/Ethyl acetate=5/1, 1/1) to afford Bz-25b (365 mg, 1.24 mmol, 39.08% yield) as white solid. ¹H NMR (CDCl₃, 400 MHz) δ7.11 (d, J=8.4 Hz, 2H), 6.84 (d, J=8.4 Hz, 2H), 3.79 (s, 3H), 3.36-3.30 (m, 2H), 3.15-3.09 (m, 2H), 2.79-2.71 (m, 2H), 1.57-1.50 (m, 2H), 1.46 (s, 9H), 0.87 (t, J=7.6 Hz, 3H).

### Preparation of N-[2-(4-methoxyphenyl)ethyl]propan-1-amine, Bz-25c

To a solution of Bz-25b (365 mg, 1.24 mmol, 1 eq) in EtOAc (5 mL) was added HCl/EtOAc (5 mL). The mixture was stirred at 25° C. for 3 h and then concentrated in vacuum to give Bz-25c.

Preparation of Bz-25: To a solution of 2-amino-8-[3-[3-(hydroxymethyl)azetidin-1-yl]sulfonylphenyl]-3H-1-benzazepine-4-carboxylic acid, Bz-21d (186 mg, 435 umol, 1 eq) in DMF (1.00 mL) was added PYAOP (340 mg, 653 umol, 1.5 eq) and DIEA (393 mg, 3.05 mmol, 531 μL, 7 eq), and then Bz-25c (100 mg, 435 umol, 1 eq, HCl) was added. The mixture was stirred at 25° C. for 3 h, and then filtered and concentrated. The residue was purified by pre-HPLC (column:Nano-micro Kromasil® C18 100*30 mm 8 um; mobile phase:[water(0.1% TFA)-ACN]; B %:25%-55%, 10 min]) to give 2-amino-8-[3-[3-(hydroxymethyl)azetidin-1-yl]sulfonylphenyl]-N-[2-(4-methoxyphenyl)ethyl]-N-propyl-3H-1-benzazepine-4-carboxamide, Bz-25 (14 mg, 23.23 umol, 5.34% yield) as a light yellow solid. ¹H NMR (MeOD-d₄, 400 MHz) δ8.13-8.03 (m, 2H), 7.93-7.87 (m, 1H), 7.84-7.80 (m, 1H), 7.79-7.74 (m, 1H), 7.69 (br s, 1H), 7.60 (br d, J=8.0 Hz, 1H), 7.08-6.51 (m, 5H), 3.86 (t, J=8.4 Hz, 2H), 3.75 (s, 4H), 3.61 (dd, J=5.8, 8.1 Hz, 2H), 3.56-3.45 (m, 1H), 3.54-3.49 (m, 1H), 3.42 (d, J=6.2 Hz, 2H), 2.93-2.87 (m, 2H), 2.65-2.47 (m, 1H), 1.75-1.68 (m, 2H), 1.03-0.94 (m, 3H). LC/MS [M+H]603.3 (calculated); LC/MS [M+H]603.3 (observed).

### Example 22 Synthesis of Bz-26

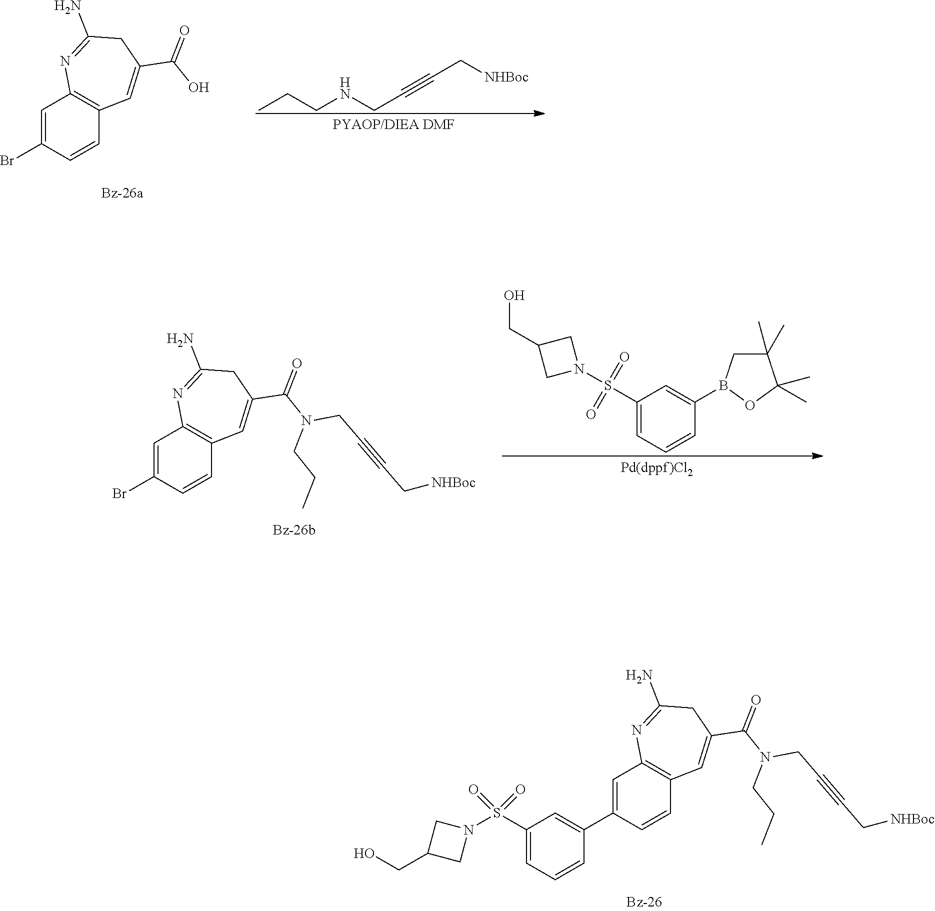 [SMILES: NC1=Nc2cc(Br)ccc2C=C(C(=O)O)C1 ; CCCNCC#CCNC(=O)OC(C)(C)C ; CCCN(CC#CCNC(=O)OC(C)(C)C)C(=O)C1=Cc2ccc(Br)cc2N=C(N)C1 ; CC1(C)CB(c2cccc(S(=O)(=O)N3CC(CO)C3)c2)OC1(C)C ; CCCN(CC#CCNC(=O)OC(C)(C)C)C(=O)C1=Cc2ccc(-c3cccc(S(=O)(=O)N4CC(CO)C4)c3)cc2N=C(N)C1]

Preparation of Bz-26b: To a mixture of 2-amino-8-bromo-3H-1-benzazepine-4-carboxylic acid, Bz-26a (0.5 g, 1.78 mmol, 1.0 eq), PYAOP (1.02 g, 1.96 mmol, 1.1 eq) and DIEA (920 mg, 7.11 mmol, 1.24 mL, 4.0 eq) in DMF (8 mL) was added tert-butyl N-[4-(propylamino)but-2-ynyl]carbamate (400 mg, 1.78 mmol, 1.0 eq) at 25° C. and then stirred for 0.5 hours at this temperature. The mixture was poured into water (40 mL). The aqueous phase was extracted with ethyl acetate (30 mL×3). The combined organic phase was washed with brine (30 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/1, 0/1) to give tert-butyl N-[4-[(2-amino-8-bromo-3H-1-benzazepine-4-carbonyl)-propyl-amino]but-2-ynyl]carbamate, Bz-26b (0.5 g, 1.02 mmol, 57.4% yield) as light yellow solid. ¹H NMR (CDCl₃, 400 MHz) δ7.52 (s, 1H), 7.39 (s, 2H), 7.07 (br s, 1H), 4.37 (s, 2H), 4.06 (d, J=5.2 Hz, 2H), 3.65 (s, 2H), 2.91 (s, 2H), 1.88-1.74 (m, 2H), 1.57 (s, 9H), 1.06 (t, J=7.2 Hz, 3H).

Preparation of Bz-26: To a mixture of [1-[3-(4,4,5,5-tetramethyl-1,3,2-dioxaborolan-2-yl)phenyl]sulfonylazetidin-3-yl]methanol (1.73 g, 4.90 mmol, 1.2 eq), Bz-26b (2.0 g, 4.09 mmol, 1.0 eq) and Pd(dppf)C₁₂ (150 mg, 204 umol, 0.05 eq) in dioxane (40 mL) was added K₂CO₃ (1.13 g, 8.17 mmol, 2 eq) in H₂O (5 mL) at 25° C. under N₂ and then stirred at 100° C. for 1 hour. The mixture was filtered and concentrated in vacuum. The residue was purified by silica gel chromatography (column height: 250 mm, diameter: 100 mm, 100-200 mesh silica gel, Petroleum ether/Ethyl acetate=1/1, 0/1) to afford tert-butyl N-[4-[[2-amino-8-[3-[3-(hydroxymethyl)azetidin-1-yl]sulfonylphenyl]-3H-1-benzazepine-4-carbonyl]-propyl-amino]but-2-ynyl]carbamate, Bz-26 (2.0 g, 3.15 mmol, 76.9% yield) as light yellow solid. ¹H NMR (MeOD, 400 MHz) δ8.07 (s, 1H), 8.04 (br d, J=7.6 Hz, 1H), 7.88-7.82 (m, 1H), 7.79-7.73 (m, 1H), 7.53-7.46 (m, 2H), 7.43-7.37 (m, 1H), 7.12 (s, 1H), 4.29 (s, 2H), 3.93-3.82 (m, 4H), 3.62-3.50 (m, 4H), 3.42 (d, J=6.4 Hz, 2H), 3.31 (s, 2H), 2.64-2.52 (m, 1H), 1.76-1.70 (m, 2H), 1.43 (s, 9H), 0.99-0.91 (m, 3H). LC/MS [M+H]636.3 (calculated); LC/MS [M+H]636.3 (observed). LCMS (ESI): mass calcd. for C₃₃H₄₁N₅O₆S 635.28, m/z found 636.3[M+H]⁺.

### Example 23 Synthesis of Bz-27

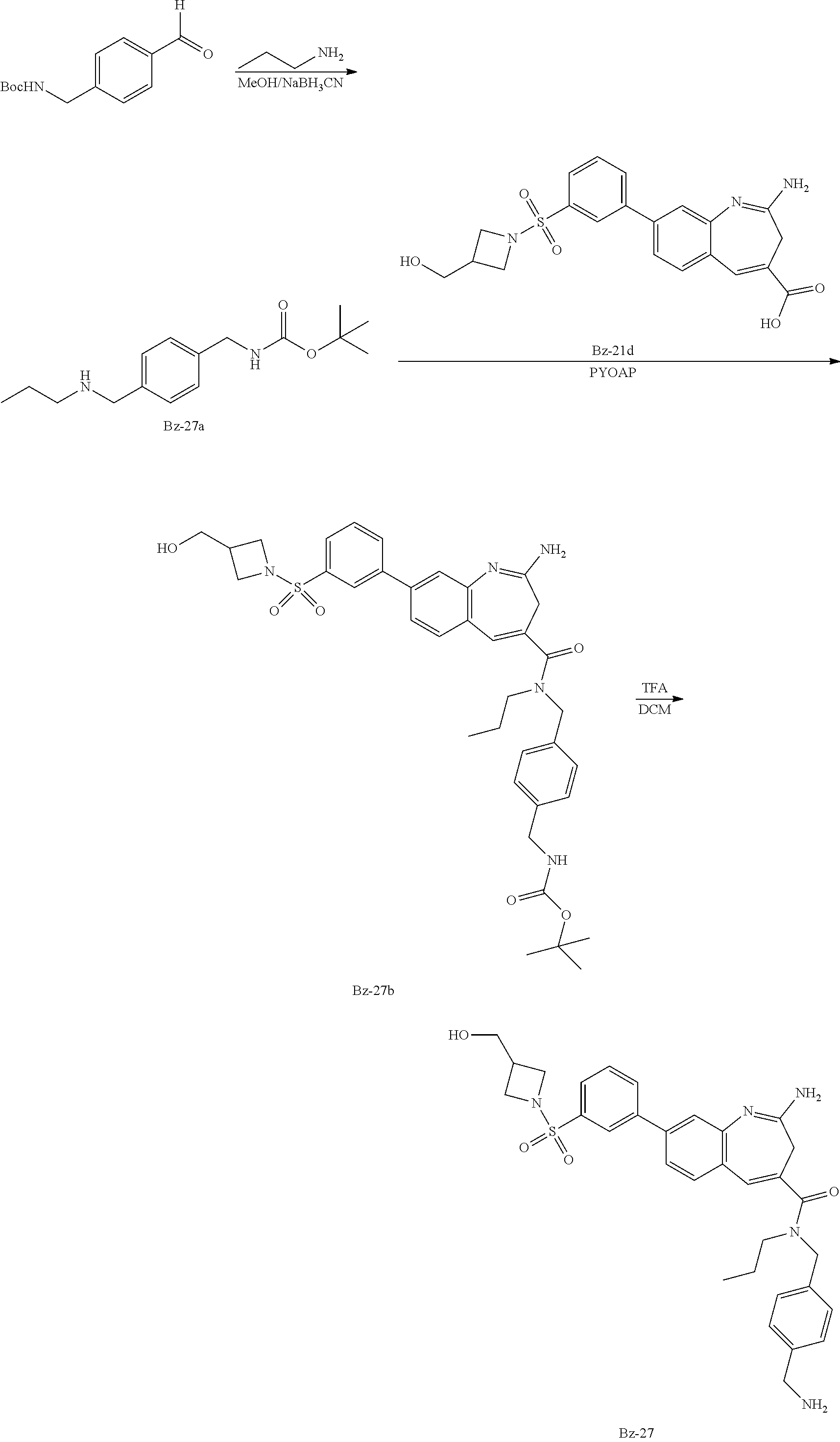

Preparation of Bz-27a: To a solution of tert-butyl N-[(4-formylphenyl)methyl]carbamate (400 mg, 1.70 mmol, 1 eq), propan-1-amine (1.00 g, 17.0 mmol, 1.40 mL, 10 eq) and AcOH (10 mg, 170 umol, 9.72 uL, 0.1 eq) in MeOH (1 mL) was added NaBH₃CN (213 mg, 3.40 mmol, 2 eq), the mixture was stirred at 25° C. for 3 h. The reaction mixture was poured into water (10 mL), and then extracted with EtOAc (5 mL×3). The combined organic layers were washed with brine (5 mL×1), dried over, filtered and concentrated under reduced pressure to give a residue. The residue was purified by prep-TLC (SiO₂, EtOAC:MeOH=5:1) to give tert-butyl-N-[[4-(propylaminomethyl)phenyl]methyl]carbamate, Bz-27a (200 mg, 718 umol, 42.26% yield) as colorless oil. ¹H NMR (MeOD-d₄, 400 MHz) δ7.43 (d, J=8.0 Hz, 2H), 7.37 (d, J=8.0 Hz, 2H), 4.24 (s, 2H), 4.17 (s, 2H), 3.00-2.96 (m, 2H), 1.77-1.67 (m, 2H), 1.44 (s, 9H), 1.01 (t, J=7.6 Hz, 3H).

Preparation of Bz-27b: To a solution of 2-amino-8-[3-[3-(hydroxymethyl)azetidin-1-yl]sulfonylphenyl]-3H-1-benzazepine-4-carboxylic acid, Bz-21d (122 mg, 287 umol, 1 eq) in DMF (0.80 mL) was added PYAOP (224 mg, 431.05 umol, 1.5 eq) and DIEA (111 mg, 862.10 umol, 150.16 uL, 3 eq). And then the tert-butyl N-[[4-(propylaminomethyl)phenyl]methyl]carbamate (80 mg, 287 umol, 1 eq) was added. The mixture was stirred at 25° C. for 3 h, which was filtered and concentrated. The residue was purified by prep-HPLC (column: Welch Xtimate C18100*25 mm*3 um; mobilephase:[water(0.1% TFA)-ACN]; B %:30%-50%, 12 min]). Compound tert-butylN-[[4-[[[2-amino-8-[3-[3-(hydroxymethyl)azetidin-1-yl]sulfonylphenyl]-3H-1-benzazepine-4-carbonyl]-propyl-amino]methyl]phenyl]methyl]carbamate (27 mg, 39.3 umol, 13.66% yield) was obtained as a light yellow solid. ¹H NMR (MeOD-d₄, 400 MHz) δ8.08 (t, J=9.6 Hz, 2H), 7.92-7.90 (m, 1H), 7.82 (t, J=8.4 Hz, 1H), 7.81-7.79 (m, 1H), 7.69-7.64 (m, 4H), 7.57 (s, 1H), 7.30-7.29 (m, 4H), 7.13 (s, 1H), 4.23 (s, 2H), 3.87 (t, J=8.4 Hz, 2H), 3.61 (t, J=6.0 Hz, 2H), 3.42-3.41 (m, 2H), 3.31 (t, J=1.6 Hz, 2H), 2.60-2.55 (m, 1H), 1.71-1.70 (m, 2H), 1.44 (s, 9H), 0.99-0.90 (m, 3H). LC/MS [M+H]688.3 (calculated); LC/MS [M+H]688.3 (observed).

Preparation of Bz-27: To a solution of Bz-27b (50 mg, 72.7 umol, 1 eq) in DCM (1 mL) was added TFA (165 mg, 1.45 mmol, 108 μL, 20 eq), and then stirred at 25° C. for 2 h. The mixture was filtered and concentrated. The residue was purified by prep-HPLC(column: Nano-micro Kromasil C18 100*30 mm8 um; mobilephase:[water(0.1% TFA)-CAN]; B %:5%-30%, 10 min]) to give 2-amino-N-[[4-(aminomethyl)phenyl]methyl]-8-[3-[3-(hydroxymethyl)azetidin-1-yl]sulfonylphenyl]-N-propyl-3H-1-benzazepine-4-carboxamide, Bz-27 (4 mg, 6.81 umol, 9.36% yield) as a white solid. ¹H NMR (MeOH-d₄, 400 MHz) δ8.13-8.03 (m, 2H), 7.91 (d, J=8.0 Hz, 1H), 7.85-7.78 (m, 1H), 7.75-7.70 (m, 2H), 7.59-7.33 (m, 5H), 7.15 (s, 1H), 4.13 (s, 2H), 3.86 (t, J=8.4 Hz, 2H), 3.61 (dd, J=6.1, 7.8 Hz, 2H), 3.48 (br d, J=7.6 Hz, 2H), 3.42 (d, J=6.2 Hz, 4H), 3.32 (br s, 1H), 3.31-3.31 (m, 1H), 3.31-3.30 (m, 2H), 2.63-2.52 (m, 1H), 1.76-1.61 (m, 2H), 0.91 (br s, 3H). LC/MS [M+H]588.3 (calculated); LC/MS [M+H]588.3 (observed).

### Example 24 Synthesis of Bz-28

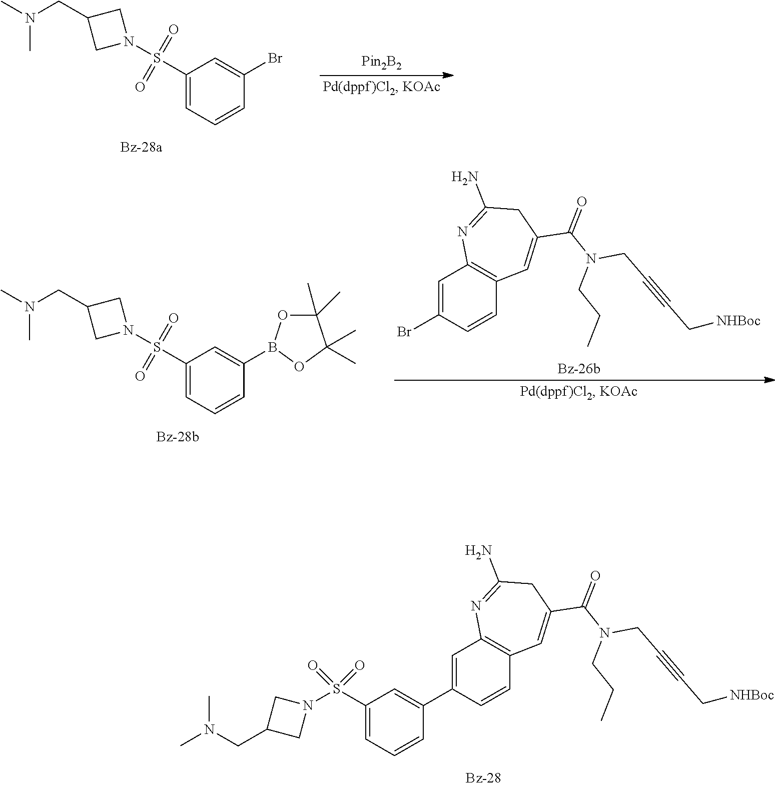

Preparation of Bz-28b: A mixture of 1-[1-(3-bromophenyl)sulfonylazetidin-3-yl]-N,N-dimethyl-methanamine, Bz-28a (0.3 g, 900.24 umol, 1 eq), Pin₂B₂(342.91 mg, 1.35 mmol, 1.5 eq), Pd(dppf)C₁₂ (32.94 mg, 45.01 umol, 0.05 eq) and KOAc (176.70 mg, 1.80 mmol, 2 eq) in dioxane (6 mL) was degassed and purged with N₂ for 3 times, and then stirred at 90° C. for 2 h under N₂ atmosphere. The reaction mixture was cooled to 25° C., and added with de-Pd silica gel (1 g) and then stirred at 25° C. for 30 min. The mixture was filtered and washed with EtOAc (10 mL×5) and concentrated under reduced pressure to give N,N-dimethyl-1-[1-[3-(4,4,5,5-tetramethyl-1,3,2-dioxaborolan-2-yl)phenyl]sulfonylazetidin-3-yl]methanamine, Bz-28b (0.6 g, crude) as a yellow oil.

Preparation of Bz-28: A mixture of Bz-28b (699 mg, 920 umol, 1.5 eq), tert-butyl N-[4-[(2-amino-8-bromo-3H-1-benzazepine-4-carbonyl)-propyl-amino]but-2-ynyl]carbamate, Bz-26b (300 mg, 613 umol, 1 eq), Pd(dppf)C₁₂ (22.4 mg, 30.6 umol, 0.05 eq) and K₂CO₃ (169 mg, 1.23 mmol, 2 eq) in dioxane (20 mL) and H₂O (2 mL) was degassed and purged with N₂ for 3 times, and then stirred at 90° C. for 2 h under N₂ atmosphere. The reaction mixture was quenched by addition of H₂O (60 mL) at 0° C., and then extracted with EtOAc(30 mL×3). The combined organic layers were washed with brine (10 mL×3), dried over Na₂SO₄, filtered and concentrated under reduced pressure. The residue was purified by column chromatography (SiO₂, Petroleum ether:Ethyl acetate=1:0 to 0:1) and then (SiO₂, EtOAc:MeOH=1:0 to 1:1) to give tert-butyl N-[4-[[2-amino-8-[3-[3-[(dimethylamino)methyl]azetidin-1-yl]sulfonylphenyl]-3H-1-benzazepine-4-carbonyl]-propyl-amino]but-2-ynyl]carbamate, Bz-28 (230 mg crude product, 347 umol, 56.61% yield) as a brown solid. ¹H NMR (MeOD-d₄, 400 MHz) δ8.16-8.06 (m, 2H), 7.97-7.90 (m, 1H), 7.89-7.65 (m, 4H), 7.34 (br s, 1H), 4.34 (s, 2H), 4.01 (t, J=8.4 Hz, 2H), 3.87 (s, 2H), 3.69 (dd, J=5.6, 8.4 Hz, 2H), 3.56 (br s, 2H), 3.39 (s, 2H), 3.33 (s, 2H), 3.03-2.89 (m, 1H), 2.82 (s, 6H), 1.81-1.67 (m, 2H), 1.43 (s, 9H), 0.97 (br t, J=6.8 Hz, 3H). LC/MS [M+H]663.3 (calculated); LC/MS [M+H]663.3 (observed).

### Example 25 Synthesis of Bz-29

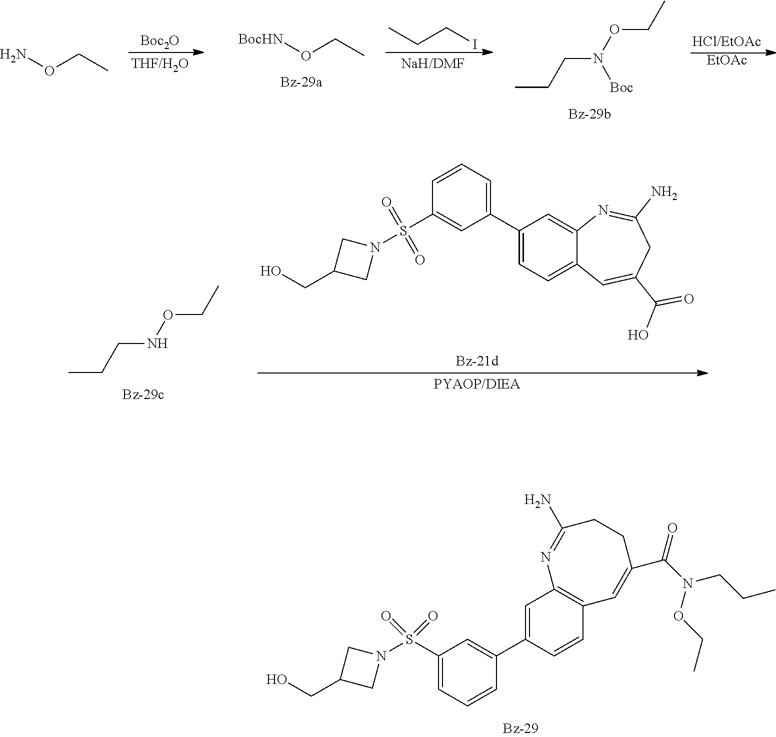

Preparation of Bz-29a: To a mixture of O-ethylhydroxylamine (3 g, 30.8 mmol, 1 eq, HCl) and Na₂CO₃ (32.6 g, 307.55 mmol, 10 eq) in DCM (30 mL) and Water (30 mL) was added tert-butoxycarbonyl tert-butyl carbonate (8.05 g, 36.9 mmol, 8.48 mL, 1.2 eq) at 25° C. and then stirred for 3 hr. The mixture was separated, and the organic layer was dried over Na₂SO₄, concentrated to residue. The crude was purified by column chromatography (SiO₂, Petroleum ether/Ethyl acetate=1:0-5:1) to give tert-butyl N-ethoxycarbamate, Bz-29a (4 g, 24.81 mmol, 80.68% yield) as colorless oil. ¹H NMR (400 MHz, CHLOROFORM-d) 63.87 (q, J=7.2 Hz, 2H), 1.45 (s, 9H), 1.20 (t, J=7.2 Hz, 3H).

Preparation of Bz-29b: To a mixture of Bz-29a (1 g, 6.20 mmol, 1 eq) in DMF (10 mL) was added NaH (298 mg, 7.44 mmol, 60% purity, 1.2 eq) at 0° C., and then stirred at 0° C. for 0.5 hr, 1-iodopropane (1.16 g, 6.82 mmol, 666.67 uL, 1.1 eq) was added to the mixture at 0° C. and it was stirred at 25° C. for 10 hr. The mixture was quenched with saturated solution of NH₄Cl (10 mL), and then extracted with EtOAc (3*10 mL). The organic layer was dried over Na₂SO₄, concentrated to give a residue. The residue was purified by column chromatography (SiO₂, Petroleum ether/Ethyl acetate=1:0-5:1) to give tert-butyl N-ethoxy-N-propyl-carbamate, Bz-29b (0.84 g, 4.13 mmol, 66.61% yield) as colorless oil. ¹H NMR (400 MHz, CHLOROFORM-d) 63.89 (q, J=7.2 Hz, 2H), 3.47-3.25 (m, 2H), 1.69-1.59 (m, 2H), 1.49 (s, 9H), 1.23 (t, J=7.2 Hz, 3H), 0.91 (t, J=7.2 Hz, 3H).

Preparation of Bz-29c: To a mixture of Bz-29b (0.84 g, 4.13 mmol, 1 eq) in EtOAc (10 mL) was added HCl/EtOAc (4 M, 5 mL, 4.84 eq). The mixture was stirred at 25° C. for 2 hr. The mixture was concentrated to give N-ethoxypropan-1-amine, Bz-29c (0.4 g, 2.86 mmol, 69.33% yield, HCl) as white solid. ¹H NMR (400 MHz, METHANOL-d₄) δ4.16 (dq, J=2.0, 7.2 Hz, 2H), 3.29-3.23 (m, 2H), 1.76 (sxt, J=7.6 Hz, 2H), 1.32 (t, J=7.2 Hz, 3H), 1.05 (t, J=7.2 Hz, 3H).

Preparation of Bz-29: To a mixture of 2-amino-8-[3-[3-(hydroxymethyl)azetidin-1-yl]sulfonylphenyl]-3H-1-benzazepine-4-carboxylic acid (200 mg, 468 umol, 1 eq) in DMF (2 mL) was added PYAOP (293 mg, 561 umol, 1.2 eq) and DIEA (181 mg, 1.40 mmol, 245 μL, 3 eq), after 3 min, N-ethoxypropan-1-amine (71.86 mg, 514.65 umol, 1.1 eq, HCl) was added. The mixture was stirred at 25° C. for 1 hr, and then concentrated to get a residue. The residue was purified by Prep-HPLC (column: Phenomenex Gemini-NX C18 75*30 mm*3 um; mobile phase: [water(10 mM NH₄HCO₃)-ACN]; B %:30%-60%, 10.5 min) to give 2-amino-N-ethoxy-8-[3-[3-(hydroxyl methyl)azetidin-1-yl]sulfonylphenyl]-N-propyl-3H-1-benzazepine-4-carboxamide, Bz-29 (3.5 mg, 6.36 umol, 1.36% yield, 93.17% purity) as white solid. ¹H NMR (400 MHz, METHANOL-d₄) δ8.10-8.02 (m, 2H), 7.89-7.73 (m, 2H), 7.53-7.48 (m, 2H), 7.46-7.40 (m, 1H), 7.31 (s, 1H), 3.95 (q, J=7.2 Hz, 2H), 3.86 (t, J=8.4 Hz, 2H), 3.74 (t, J=7.2 Hz, 2H), 3.60 (dd, J=6.4, 8.2 Hz, 2H), 3.41 (d, J=6.4 Hz, 2H), 3.34-3.31 (m, 2H), 2.67-2.43 (m, 1H), 1.77 (sxt, J=7.2 Hz, 2H), 1.18 (t, J=7.2 Hz, 3H), 0.99 (t, J=7.6 Hz, 3H). LC/MS [M+H]513.2 (calculated); LC/MS [M+H]513.4 (observed).

### Example 26 Synthesis of Bz-30

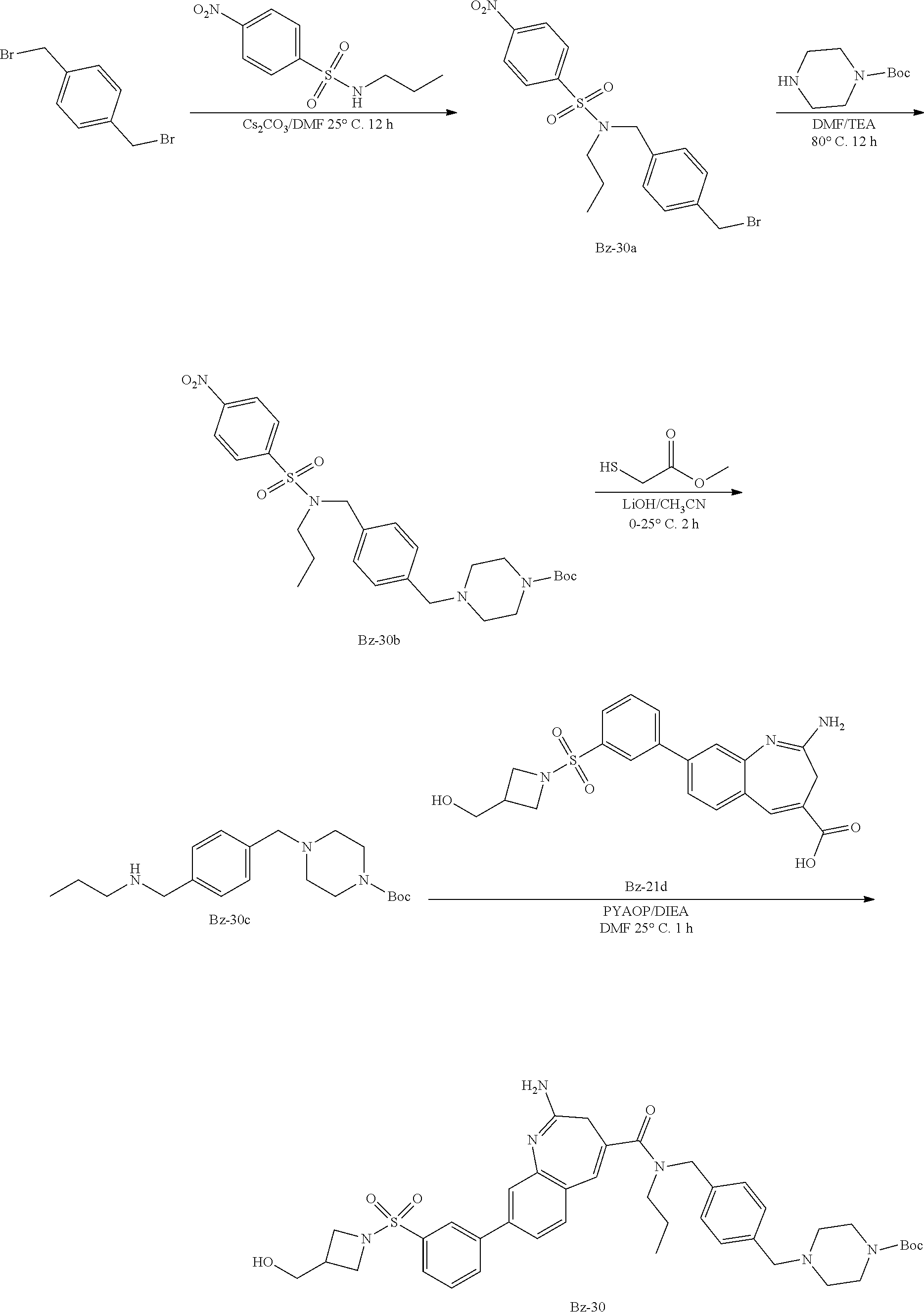

Preparation of Bz-30a: To a mixture of 1,4-bis(bromomethyl)benzene (6.48 g, 24.6 mmol, 2.0 eq) and 4-nitro-N-propyl-benzenesulfonamide (3.0 g, 12.3 mmol, 1.0 eq) in DMF (40 mL) was added Cs₂CO₃ (4.80 g, 14.7 mmol, 1.2 eq) in one portion at 25° C. and then stirred for 12 h. The reaction was diluted with water (100 mL) and extracted with EtOAc (50 mL×3). The organic layer was washed with brine, dried over Na₂SO₄ filtered and concentrated. The residue was purified by silica gel chromatography (Petroleum ether/Ethyl acetate=I/O, 3/1) to afford N-[[4-(bromomethyl)phenyl]methyl]-4-nitro-N-propyl-benzenesulfonamide, Bz-30a (1.5 g, 3.51 mmol, 28.6% yield) as white solid. ¹H NMR (CDCl₃, 400 MHz) δ8.35 (d, J=8.8 Hz, 2H), 7.98 (d, J=8.8 Hz, 2H), 7.35 (d, J=8.0 Hz, 2H), 7.24 (d, J=8.0 Hz, 2H), 4.48 (s, 2H), 4.40 (s, 2H), 3.19-3.11 (m, 2H), 1.42 (m, 2H), 0.76 (t, J=7.6 Hz, 3H).

Preparation of Bz-30b: To a mixture of Bz-30a (1.3 g, 3.04 mmol, 1.0 eq) and tert-butyl piperazine-1-carboxylate (2.27 g, 12.2 mmol, 4.0 eq) in DMF (15 mL) was added Et₃N (1.23 g, 12.2 mmol, 1.69 mL, 4.0 eq) at 25° C. and then stirred at 80° C. for 12 h. The mixture was diluted with water (50 mL) and extracted with EtOAc (50 mL×3). The organic layer was washed with brine, dried over Na₂SO₄, filtered and concentrated. The residue was purified by silica gel chromatography (Petroleum ether/Ethyl acetate=I/O, 3/1) to afford tert-butyl 4-[[4-[[(4-nitrophenyl)sulfonyl-propyl-amino]methyl]phenyl]methyl]piperazine-1-carboxylate, Bz-30b (1.7 g, crude) as yellow solid. ¹H NMR (DMSO, 400 MHz) δ8.39 (d, J=8.8 Hz, 2H), 8.11 (d, J=8.8 Hz, 2H), 7.21 (s, 4H), 4.36 (s, 2H), 3.45 (s, 2H), 3.31-2.27 (m, 4H), 3.12-3.05 (m, 2H), 2.28-2.26 (m, 4H), 1.38 (s, 9H), 1.33-1.25 (m, 2H), 0.65 (t, J=7.6 Hz, 3H). Preparation of Bz-30c: To a solution of Bz-30b (1.0 g, 1.88 mmol, 1.0 eq) in CH₃CN (6 mL) was added LiOH·H₂O (473 mg, 11.3 mmol, 6.0 eq) in one portion at 0° C. Then methyl 2-sulfanylacetate (598 mg, 5.63 mmol, 511 μL, 3.0 eq) was added and it was stirred at 25° C. for 2 h. The mixture was filtered and concentrated. The residue was diluted with MTBE (5 ml) and then adjusted the pH of the mixture to about 2 with aq. HCl (1M), extracted with MTBE (20 mL) (discarded). The aqueous phase was adjusted pH=9 with aq·NaHCO₃ and then extracted with EtOAc (30 mL×3). The organic layer was washed with brine, dried over Na₂SO₄, filtered and concentrated to obtain tert-butyl 4-[[4-(propylaminomethyl)phenyl]methyl]piperazine-1-carboxylate, Bz-30c (0.5 g, crude) as yellow oil. ¹H NMR (MeOD, 400 MHz) δ7.32-7.30 (m, 4H), 3.73 (s, 2H), 3.53 (s, 2H), 3.43-3.40 (m, 4H), 2.57-2.50 (m, 2H), 2.41-2.48 (m, 4H), 1.58-1.51 (m, 2H), 1.45 (s, 9H), 0.92 (t, J=7.6 Hz, 3H).

Preparation of Bz-30: To a mixture of 2-amino-8-[3-[3-(hydroxymethyl)azetidin-1-yl]sulfonylphenyl]-3H-1-benzazepine-4-carboxylic acid, Bz-21d (400 mg, 936 umol, 1.0 eq) in DMF (8 mL) was added PYAOP (585 mg, 1.12 mmol, 1.2 eq), DIEA (363 mg, 2.81 mmol, 489 μL, 3.0 eq) and Bz-30c (358 mg, 1.03 mmol, 1.1 eq) in one portion at 25° C. and then stirred for 1 h. The mixture was filtered and concentrated. The residue was purified by prep-HPLC (column: Phenomenex Luna C18 100*30 mm*5 um; mobile phase: [water (0.1% TFA) -ACN]; B %: 15%-45%, 10 min) to give tert-butyl 4-[[4-[[[2-amino-8-[3-[3-(hydroxymethyl)azetidin-1-yl]sulfonylphenyl]-3H-1-benzazepine-4-carbonyl]-propyl-amino]methyl]phenyl]methyl]piperazine-1-carboxylate, Bz-30 (0.35 g, 462 umol, 49.4% yield) as white solid. ¹H NMR (MeOD, 400 MHz) δ 8.14-8.05 (m, 2H), 7.92 (d, J=7.6 Hz, 1H), 7.82 (t, J=7.6 Hz, 1H), 7.78-7.69 (m, 2H), 7.63-7.42 (m, 5H), 7.17 (s, 1H), 4.37 (s, 2H), 3.86 (t, J=8.0 Hz, 2H), 3.61 (dd, J=6.0, 8.0 Hz, 2H), 3.53-3.49 (m, 2H), 3.43-3.41 (m, 6H), 3.31-3.29 (m, 8H), 2.63-2.54 (m, 1H), 1.76-1.65 (m, 2H), 1.47 (s, 9H), 0.95-0.89 (m, 3H). LC/MS [M+H]757.4 (calculated); LC/MS [M+H]757.4 (observed).

### Example 27 Synthesis of Bz-31

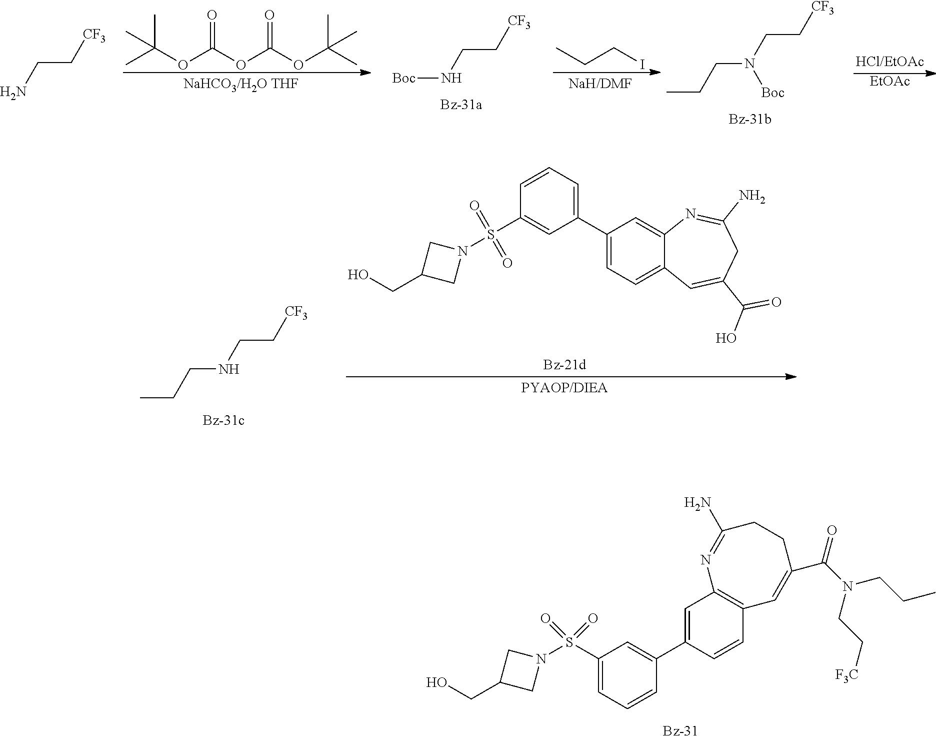

Preparation of Bz-31a: To a mixture of 3,3,3-trifluoropropan-1-amine (0.5 g, 3.34 mmol, 1 eq, HCl) and NaHCO₃ (842.64 mg, 10.03 mmol, 390.11 uL, 3 eq) in THE (3 mL) and H₂O (3 mL) was added tert-butoxycarbonyl tert-butyl carbonate (730 mg, 3.34 mmol, 768 μL, 1 eq), and then stirred at 25° C. for 1 h under N₂ atmosphere. The mixture was poured into H₂O (15 mL), extracted with ethyl acetate (15 mL×3). The combined organic phase was washed with brine (15 mL), dried with anhydrous Na₂SO₄, filtered and concentrated in vacuum. The crude product was purified by silica gel chromatography eluted with (Petroleum ether:Ethyl acetate=5:0 to 1:1) to give tert-butyl N-(3,3,3-trifluoropropyl)carbamate, Bz-31a (500 mg, 2.35 mmol, 70.14% yield) as a colorless oil. ¹H NMR (CDCl₃, 400 MHz) δ4.75 (br s, 1H), 3.40 (q, J=6.4 Hz, 2H), 2.40-2.27 (m, 2H), 1.45 (s, 9H).

Preparation of Bz-31b: To a solution of Bz-31a (400 mg, 1.88 mmol, 1 eq) in DMF (5 mL) was added NaH (113 mg, 2.81 mmol, 60% purity, 1.5 eq) at 0° C. After 30 min, 1-iodopropane (637.88 mg, 3.75 mmol, 366 μL, 2 eq) was added to the mixture and then stirred at 20° C. for 2 h. The reaction mixture was quenched at 0° C. by the addition of saturated NH₄Cl (10 mL), then extracted with EtOAc (10 mL×3). The organic phase was dried with anhydrous Na₂SO₄, filtered and concentrated in vacuum. The reaction mixture was purified by silica gel column chromatography (Petroleum ether:Ethyl acetate=5:1 to 1:1). Compound tert-butyl N-propyl-N-(3,3,3-trifluoropropyl)carbamate, Bz-31b (400 mg, 1.57 mmol, 83.52% yield) was obtained as a colorless oil. ¹H NMR (CDCl₃, 400 MHz) δ3.41 (t, J=7.2 Hz, 2H), 3.19-3.12 (m, 1H), 2.40-2.32 (m, 2H), 1.58-1.50 (m, 2H), 1.47 (s, 9H), 0.89 (t, J=7.6 Hz, 3H).

Preparation of Bz-31c: To a solution of tert-butyl N-propyl-N-(3,3,3-trifluoropropyl)carbamate (400 mg, 1.57 mmol, 1 eq) in EtOAc (3 mL) was added HCl/EtOAc (4 M, 5.88 mL, 15 eq) and then stirred at 20° C. for 2 h. The mixture was filtered and concentrated in vacuum to give 3,3,3-trifluoro-N-propyl-propan-1-amine, Bz-31c (240 mg, crude, HCl) as a white solid. ¹H NMR (MeOD-d₄, 400 MHz) δ3.34-3.31 (m, 2H), 3.06-3.00 (m,

Preparation of Bz-31: a solution of 2-amino-8-[3-[3-(hydroxymethyl)azetidin-1-yl]sulfonylphenyl]-3H-1-benzazepine-4-carboxylic acid, Bz-21d (100 mg, 233 umol, 1 eq), DIEA (90.7 mg, 702 umol, 122 μL, 3 eq) and PYAOP (183 mg, 351 umol, 1.5 eq) in DMF (1 mL) was added Bz-31c (44.8 mg, 234 umol, 1 eq, HCl), and then stirred at 20° C. for 1 h. The mixture was filtered and concentrated in vacuum. The residue was purified by prep-HPLC (column: Waters Xbridge BEH C18 100*30 mm*10 um; mobile phase: [water(10 mM NH₄HCO₃)-ACN]; B %: 30%-60%, 8 min) to afford 2-amino-8-[3-[3-(hydroxymethyl)azetidin-1-yl]sulfonylphenyl]-N-propyl-N-(3,3,3-trifluoropropyl)-3H-1-benzazepine-4-carboxamide, Bz-31 (7 mg, 12.40 umol, 5.30% yield) as a white solid. ¹H NMR (MeOD-d₄, 400 MHz) δ8.07 (s, 1H), 8.04 (br d, J=7.6 Hz, 1H), 7.86-7.81 (m, 1H), 7.80-7.73 (m, 1H), 7.49-7.44 (m, 2H), 7.42-7.37 (m, 1H), 6.94 (s, 1H), 3.86 (t, J=8.4 Hz, 2H), 3.73 (br s, 2H), 3.60 (dd, J=6.0, 8.0 Hz, 2H), 3.52-3.45 (m, 2H), 3.42 (d, J=6.4 Hz, 2H), 3.33-3.32 (m, 2H), 2.68-2.53 (m, 3H), 1.74-1.64 (m, 2H), 0.91 (br s, 3H). LC/MS [M+H]565.2 (calculated); LC/MS [M+H]565.3 (observed).

### Example 28 Synthesis of Bz-32

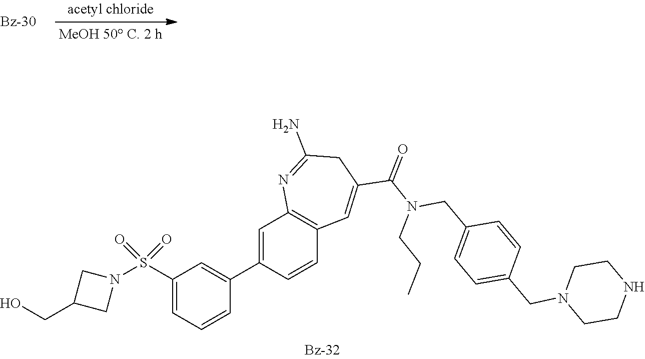 [SMILES: CCCN(Cc1ccc(CN2CCNCC2)cc1)C(=O)C1=Cc2ccc(-c3cccc(S(=O)(=O)N4CC(CO)C4)c3)cc2N=C(N)C1]

Preparation of Bz-32: To a solution of tert-butyl 4-[[4-[[[2-amino-8-[3-[3-(hydroxymethyl)azetidin-1-yl]sulfonylphenyl]-3H-1-benzazepine-4-carbonyl]-propyl-amino]methyl]phenyl]methyl]piperazine-1-carboxylate, Bz-30 (0.16 g, 211 umol, 1.0 eq) in MeOH (10 mL) was added acetyl chloride (49.8 mg, 634 umol, 45.3 uL, 3.0 eq) at 25° C. and it was stirred at 50° C. for 2 h. The mixture was concentrated in vacuum, and the residue was purification by prep-HPLC (column: Waters Xbridge BEH C18 100*25 mm*5 um; mobile phase: [water(10 mM NH₄HCO₃)-ACN]; B %: 25%-55%, 10 min) to give 2-amino-8-[3-[3-(hydroxymethyl)azetidin-1-yl]sulfonylphenyl]-N-[[4-(piperazin-1-ylmethyl)phenyl]methyl]-N-propyl-3H-1-benzazepine-4-carboxamide, Bz-32 (36 mg, 54.8 umol, 25.9% yield) as white solid. ¹H NMR (MeOD, 400 MHz) δ8.06 (s, 1H), 8.02 (d, J=7.6 Hz, 1H), 7.83 (d, J=8.0 Hz, 1H), 7.79-7.72 (m, 1H), 7.46 (s, 2H), 7.40-7.22 (m, 5H), 6.93 (s, 1H), 4.74 (s, 2H), 3.85 (t, J=8.4 Hz, 2H), 3.62-3.56 (m, 2H), 3.52 (s, 2H), 3.45-3.34 (m, 4H), 2.85 (t, J=4.4 Hz, 4H), 2.66-2.52 (m, 2H), 2.48-2.44 (m, 4H), 1.72-1.60 (m, 2H), 0.90-0.88 (m, 3H). LC/MS [M+H]657.3 (calculated); LC/MS [M+H]657.5 (observed).

### Example 29 Synthesis of Bz-33

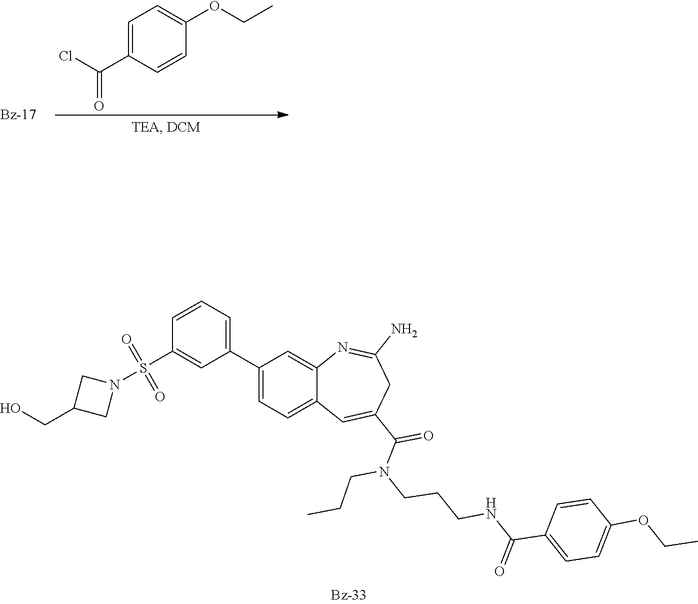 [SMILES: CCOc1ccc(C(=O)Cl)cc1 ; CCCN(CCCNC(=O)c1ccc(OCC)cc1)C(=O)C1=Cc2ccc(-c3cccc(S(=O)(=O)N4CC(CO)C4)c3)cc2N=C(N)C1]

2-Amino-N-(3-aminopropyl)-8-(3-((3-(hydroxymethyl)azetidin-1-yl)sulfonyl)phenyl)-N-propyl-3H-benzo[b]azepine-4-carboxamide, Bz-17 (0.01 g, 0.019 mmol, 1 eq.) was dissolved in DCM. Triethylamine (4 μl, 0.029 mmol, 1.5 eq.) was added, followed by 4-ethoxybenzoyl chloride (0.004 g, 0.019 mmol, 1 eq.). The reaction was stirred at room temperature, then concentrated and purified by HPLC to give 2-amino-N-(3-(4-ethoxybenzamido)propyl)-8-(3-((3-(hydroxymethyl)azetidin-1-yl)sulfonyl)phenyl)-N-propyl-3H-benzo[b]azepine-4-carboxamide, Bz-33 (0.0028 g, 0.0042 mmol, 22%). LC/MS [M+H]674.30 (calculated); LC/MS [M+H]674.74 (observed).

### Example 30 Synthesis of Bz-34

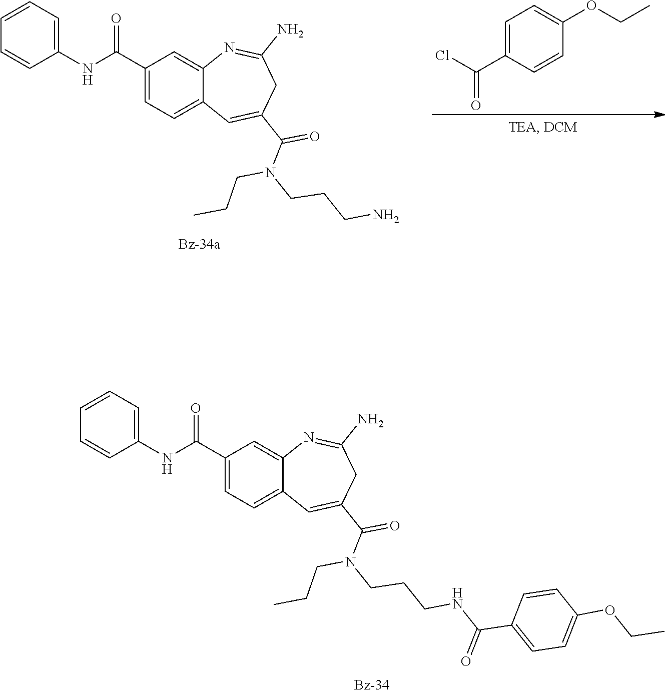 [SMILES: CCCN(CCCN)C(=O)C1=Cc2ccc(C(=O)Nc3ccccc3)cc2N=C(N)C1 ; CCOc1ccc(C(=O)Cl)cc1 ; CCCN(CCCNC(=O)c1ccc(OCC)cc1)C(=O)C1=Cc2ccc(C(=O)Nc3ccccc3)cc2N=C(N)C1]

2-Amino-N⁴-(3-aminopropyl)-N′-phenyl-N⁴-propyl-3H-benzo[b]azepine-4,8-dicarboxamide, Bz-34a (0.01 g, 0.024 mmol, 1 eq.) was dissolved in DCM. Triethylamine (5 μl, 0.036 mmol, 1.5 eq.) was added, followed by 4-ethoxybenzoyl chloride (0.004 g, 0.024 mmol, 1 eq.). The reaction was stirred at room temperature, then concentrated and purified by HPLC to give 2-amino-N⁴-(3-(4-ethoxybenzamido)propyl)-N⁸-phenyl-N⁴-propyl-3H-benzo[b]azepine-4,8-dicarboxamide, Bz-34 (0.005 g, 0.009 mmol, 38%). LC/MS [M+H]568.29 (calculated); LC/MS [M+H]568.50 (observed).

Preparation of Aminobenzazepine-linker Formula II compounds (BzL) and intermediates

### Example 31 Synthesis of BzL-1

Following the procedures described herein, ethyl 2-amino-8-(3-((2-(2-(3-oxo-3-(2,3,5,6-tetrafluorophenoxy)propoxy)ethoxy)ethyl)carbamoyl)phenyl)-3H-benzo[b]azepine-4-carboxylate, BzL-1 was prepared and characterized.

### Example 32 Synthesis of BzL-2

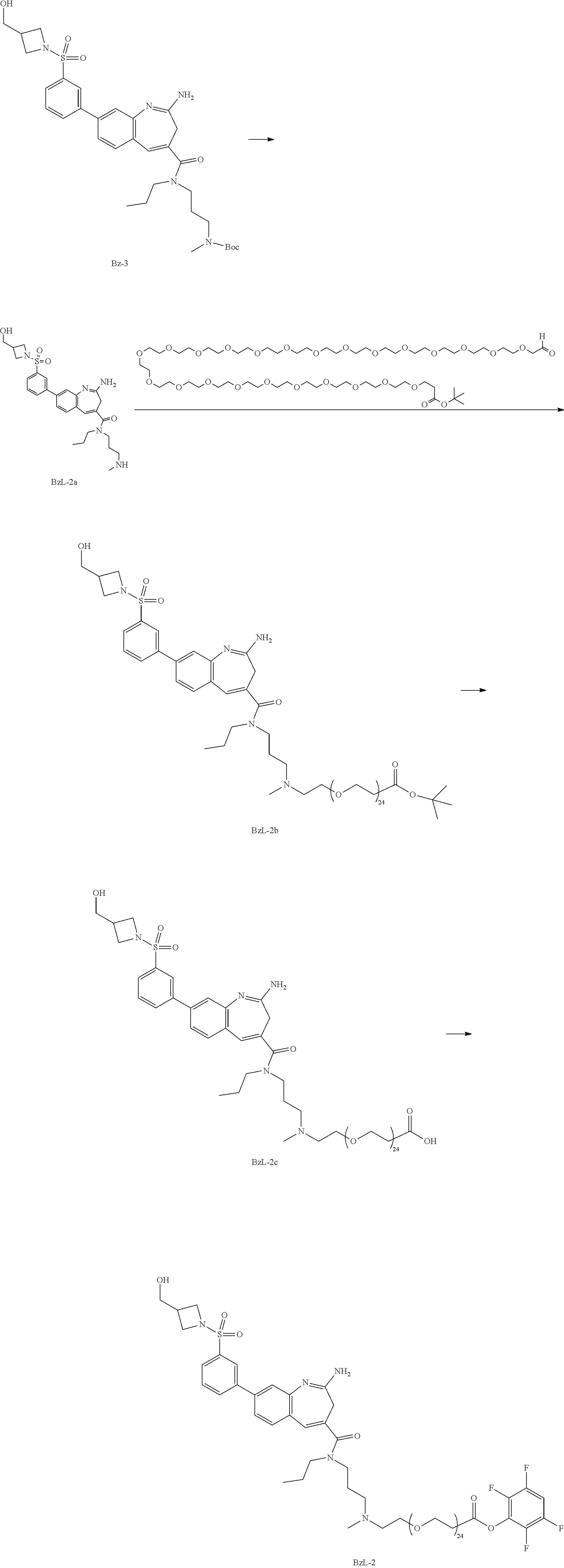

### Synthesis of 2-amino-8-(3-((3-(hydroxymethyl)azetidin-1-yl)sulfonyl)phenyl)-N-(3-(methylamino)propyl)-N-propyl-3H-benzo[b]azepine-4-carboxamide, BzL-2a

BzL-2a was synthesized from Bz-3 according to the procedure described for Bz-11a. LC/MS [M+H]540.26 (calculated); LC/MS [M+H]540.53 (observed).

Synthesis of tert-butyl 80-(2-amino-8-(3-((3-(hydroxymethyl)azetidin-1-yl)sulfonyl)phenyl)-3H-benzo[b]azepine-4-carbonyl)-76-methyl-4,7,10,13,16,19,22,25,28,31,34,37,40,43,46,49,52,55,58,61,64,67,70,73-tetracosaoxa-76,80-diazatrioctacontanoate, BzL-2b.

A vial was charged with BzL-2a (15.1 mg, 0.028 mmol), tert-butyl 1-oxo-3,6,9,12,15,18,21,24,27,30,33,36,39,42,45,48,51,54,57,60,63,66,69,72-tetracosaoxapentaheptacontan-75-oate (0.042 mmol), sodium triacetoxyborohydride (30 mg, 0.14 mmol) in 100 μL DMF. The reaction was stirred for 5 h, upon which 100 μL of 10% sodium carbonate was added and stirred for 1 h. The mixture was filtered and purified by reverse phase preparative HPLC utilizing a 25-75% gradient of acetonitrile:water containing 0.1% trifluoroacetic acid. The purified fractions were combined and lyophilized to afford 40.7 mg of BzL-2b in 84% yield. LC/MS [M+H]1724.98 (calculated); LC/MS [M+H]1726.52 (observed).

Synthesis of 80-(2-amino-8-(3-((3-(hydroxymethyl)azetidin-1-yl)sulfonyl)phenyl)-3H-benzo[b]azepine-4-carbonyl)-76-methyl-4,7,10,13,16,19,22,25,28,31,34,37,40,43,46,49,52,55,58,61,64,67,70,73-tetracosaoxa-76,80-diazatrioctacontanoic acid, BzL-2c.

A vial was charged with BzL-2b (18 mg, 0.010 mmol), 300 μL DCM, and 100 μL trifluoroacetic acid. The reaction was maintained for 45 min, concentrated under vacuum, and azeotroped thrice with 1 mL toluene. The reaction was taken forward without any further purification.

2,3,5,6-Tetrafluorophenyl 80-(2-amino-8-(3-((3-(hydroxymethyl)azetidin-1-yl)sulfonyl)phenyl)-3H-benzo[b]azepine-4-carbonyl)-76-methyl-4,7,10,13,16,19,22,25,28,31,34,37,40,43,46,49,52,55,58,61,64,67,70,73-tetracosaoxa-76,80-diazatrioctacontanoate, BzL-2 was synthesized according to the procedure described for BzL-22. LC/MS [M+H]1816.91 (calculated); LC/MS [M+H]1818.51 (observed).

### Example 33 Synthesis of BzL-3

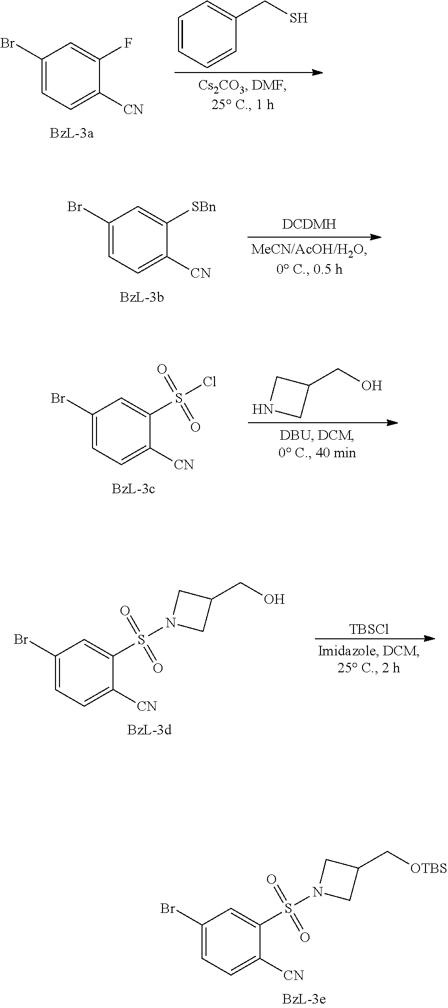

### Synthesis of 2-benzylsulfanyl-4-bromo-benzonitrile, BzL-3b

To a mixture of phenylmethanethiol (3.10 g, 25.00 mmol, 2.93 mL, 1 eq) and 4-bromo-2-fluoro-benzonitrile, BzL-3a (5 g, 25.00 mmol, 1 eq) in DMF (10 mL) was added Cs₂CO₃ (12.22 g, 37.50 mmol, 1.5 eq) at 25° C. The mixture was stirred at 25° C. for 1 hour. TLC and LCMS showed the reaction was completed. The mixture was poured into ice water (100 mL), stirred for 5 min and filtered to give BzL-3b (4 g, 13.15 mmol, 52.60% yield) as a white solid which was used into next step without further purification. ¹H NMR (CDCl₃, 400 MHz) δ 7.50 (d, J=2.0 Hz, 1H), 7.47-7.43 (m, 1H), 7.41-7.38 (m, 1H), 7.35-7.28 (m, 5H), 4.23 (s, 2H).

### Synthesis of 5-bromo-2-cyano-benzenesulfonyl chloride, BzL-3c

To a mixture of 2-benzylsulfanyl-4-bromo-benzonitrile (1 g, 3.29 mmol, 1 eq) in CH₃CN (20 mL), AcOH (0.7 mL) and H₂O (0.5 mL) was added 1,3-dichloro-5,5-imethyl-imidazolidine-2,4-dione (1.30 g, 6.57 mmol, 2 eq) in portions at 0° C. The mixture was stirred at 0° C. for 30 min. TLC and LCMS showed the reaction was completed. The mixture was poured into ice water (50 mL) and stirred for 2 min. The aqueous phase was extracted with DCM (20 mL×2). The combined organic phase was washed with brine (30 mL), dried with anhydrous Na₂SO₄, filtered and concentrated in vacuum. The residue was purified by silica gel chromatography (Petroleum ether/Ethyl acetate=20/1, 10/1) to afford BzL-3c (0.8 g, 2.85 mmol, 86.75% yield) as a white solid. ¹H NMR (CDCl₃, 400 MHz) δ 8.34 (d, J=2.0 Hz, 1H), 7.99 (dd, J=8.4, 2.0 Hz, 1H), 7.83 (d, J=8.4 Hz, 1H).

### Synthesis of 4-bromo-2-[3-(hydroxymethyl)azetidin-1-yl]sulfonyl-benzonitrile, BzL-3d

To a mixture of azetidin-3-ylmethanol (1.54 g, 12.48 mmol, 1 eq, HCl) in DCM (100 mL) was added DBU (3.80 g, 24.95 mmol, 3.76 mL, 2 eq) dropwise at 0° C. and stirred for 10 min. The mixture was added 5-bromo-2-cyano-benzenesulfonyl chloride, BzL-3c (3.5 g, 12.48 mmol, 1 eq) and stirred at 0° C. for 30 min. TLC showed the reaction was completed. The mixture was poured into ice water (100 mL) and stirred for 2 min. The aqueous phase was extracted with DCM (50 mL×3). The combined organic phase was washed with brine (20 mL), dried with anhydrous Na₂SO₄, filtered and concentrated to obtain BzL-3d (3.5 g, crude) as colorless oil which was used into the next step without further purification.

### Synthesis of 4-bromo-2-[3-[[tert-butyl(dimethyl)silyl]oxymethyl]azetidin-1-yl]sulfonyl-benzonitrile, BzL-3e

To a mixture of 4-bromo-2-[3-(hydroxymethyl)azetidin-1-yl]sulfonyl-benzonitrile, BzL-3d (3.5 g, 10.57 mmol, 1 eq) and tert-butyldimethylsilyl chloride, TBSCl (1.91 g, 12.68 mmol, 1.55 mL, 1.2 eq) in DCM (30 mL) was added imidazole (1.08 g, 15.85 mmol, 1.5 eq) in one portion at 25° C. The mixture was stirred at 25° C. for 2 hours. LCMS showed the reaction was completed. The mixture was poured into ice water (200 mL) and stirred for 2 min. The aqueous phase was extracted with DCM (100 mL×3). The combined organic phase was washed with brine (50 mL), dried with anhydrous Na₂SO₄, filtered and concentrated in vacuum. The residue was purified by silica gel chromatography (Petroleum ether/Ethyl acetate=20/1, 10/1) to afford BzL-3e (3.8 g, 8.53 mmol, 80.72% yield) as colorless oil. ¹H NMR (CDCl₃, 400 MHz) δ 8.20 (d, J=2.0 Hz, 1H), 7.82 (dd, J=8.4, 2.0 Hz, 1H), 7.72 (d, J=8.4 Hz, 1H), 4.10-4.06 (m, 2H), 3.96-3.93 (m, 2H), 3.68 (d, J=5.2 Hz, 2H), 2.82-2.76 (m, 1H), 0.86 (s, 9H), 0.00 (s, 6H).

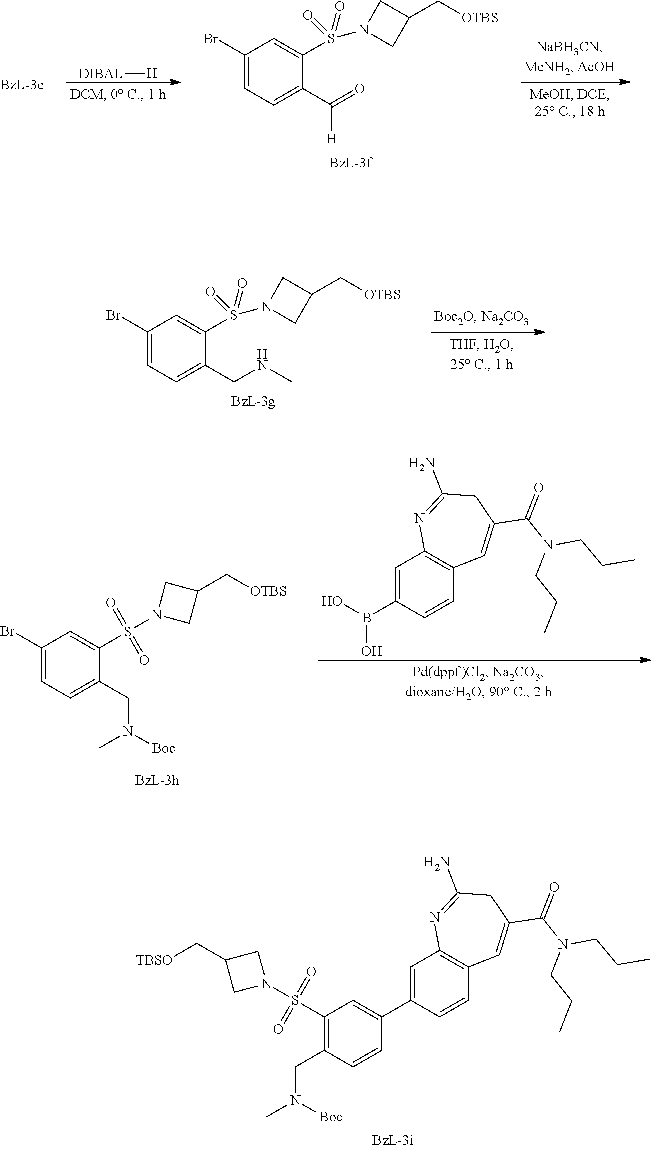

### Synthesis of 4-bromo-2-[3-[[tert-butyl(dimethyl)silyl]oxymethyl]azetidin-1-yl]sulfonyl-benzaldehyde, BzL-3f

To a solution of 4-bromo-2-[3-[[tert-butyl(dimethyl)silyl]oxymethyl]azetidin-1-yl]sulfonyl-benzonitrile, BzL-3e (3.8 g, 8.53 mmol, 1 eq) in DCM (100 mL) was added diisobutylaluminum hydride, DIBAL-H (1 M, 9.38 mL, 1.1 eq) dropwise at 0° C. under N₂. The mixture was stirred at 0° C. for 1 hour. LCMS showed the reaction was completed. The mixture was added saturated aqueous NH₄Cl (3 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=20/1, 5/1) to give BzL-3f (3.5 g, 7.80 mmol, 91.49% yield) as a light yellow oil. ¹H NMR (CDCl₃, 400 MHz) δ 10.69 (s, 1H), 8.16 (d, J=1.6 Hz, 1H), 7.97 (d, J=8.4 Hz, 1H), 7.86 (dd, J=1.6, 8.4 Hz, 1H), 3.95-3.88 (m, 2H), 3.81-3.76 (m, 2H), 3.65-3.64 (m, 2H), 2.85-2.71 (m, 1H), 0.85 (s, 8H), 0.03 (s, 6H).

### Synthesis of 1-[4-bromo-2-[3-[[tert-butyl(dimethyl)silyl]oxymethyl]azetidin-1-yl]sulfonyl-phenyl]-N-methyl-methanamine, BzL-3g

To a solution of methanamine (4.16 g, 40.14 mmol, 5 eq) (30% in MeOH) and 4-bromo-2-[3-[[tert-butyl(dimethyl)silyl]oxymethyl]azetidin-1-yl]sulfonyl-benzaldehde, BzL-3f (3.6 g, 8.03 mmol, 1 eq) in MeOH (15 mL) and DCE (15 mL) was added AcOH(482.08 mg, 8.03 mmol, 459.12 μL, 1 eq) and NaBH₃CN (1.26 g, 20.07 mmol, 2.5 eq). The mixture was stirred at 25° C. for 18 hrs. The mixture was added a few drops of water and concentrated. The residue was purified by column chromatography (SiO₂, Petroleum ether/Ethyl acetate=1:1) to obtain BzL-3g (2 g, 4.31 mmol, 53.75% yield) as colorless oil. ¹H NMR (DMSO-d₆, 400 MHz) δ 8.09-8.06 (m, 1H), 8.01-7.99 (m, 1H), 7.71 (d, J=8.4 Hz, 1H), 4.27 (s, 2H), 3.85-3.80 (m, 2H), 3.62-3.58 (m, 2H), 3.55 (d, J=5.2 Hz, 2H), 2.69-2.75 (m, 1H), 2.56 (s, 3H), 0.82 (s, 9H), 0.00 (s, 6H)

### Synthesis of tert-butyl N-[[4-bromo-2-[3-[[tert-butyl(dimethyl)silyl]oxymethyl]azetidin-1-yl]sulfonyl-phenyl]methyl]-N-methyl-carbamate, BzL-3h

To a mixture of 1-[4-bromo-2-[3-[[tert-butyl(dimethyl)silyl]oxymethyl]azetidin-1-yl]sulfonyl-phenyl]-N-methyl-methanamine, BzL-3g (2 g, 4.31 mmol, 1 eq) in THE (15 mL) and H₂O (3 mL) was added Na₂CO₃ (914.68 mg, 8.63 mmol, 2 eq) and Boc₂O (1.41 g, 6.47 mmol, 1.49 mL, 1.5 eq) in one portion at 25° C. The mixture was stirred at 25° C. for 1 hr. The mixture was poured into ice water (10 mL) and stirred for 1 min. The aqueous phase was extracted with ethyl acetate (10 mL×3). The combined organic phase was washed with brine (20 mL), dried with anhydrous Na₂SO₄, filtered and concentrated in vacuum. The residue was purified by flash silica gel chromatography (ISCO®; 2 g SepaFlash® Silica Flash Column, Eluent of 0-50% Ethyl acetate/Petroleum ether gradient at 45 mL/min) to give BzL-3h (1.4 g, 2.48 mmol, 57.57% yield) was obtained as colorless oil. ¹H NMR (DMSO-d₆, 400 MHz) δ 8.00-7.99 (m, 2H), 7.23 (d, J=8.4 Hz, 1H), 4.66 (s, 2H), 3.85-3.79 (m, 2H), 3.61-3.57 (m, 4H), 2.85 (s, 3H), 2.51-2.49 (m, 1H), 1.47-1.31 (m, 9H), 0.81 (s, 9H), −0.01 (s, 6H)

### Synthesis of tert-butyl N-[[4-[2-amino-4-(dipropylcarbamoyl)-3H-1-benzazepin-8-yl]-2-[3-[[tert-butyl(dimethyl)silyl]oxymethyl]azetidin-1-yl]sulfonyl-phenyl]methyl]-N-methyl-carbamate, BzL-3i

To a mixture of [2-amino-4-(dipropylcarbamoyl)-3H-1-benzazepin-8-yl]boronic acid (360 mg, 1.09 mmol, 1 eq) and tert-butyl N-[[4-bromo-2-[3-[[tert-butyl(dimethyl)silyl]oxymethyl]azetidin-1-yl]sulfonyl-phenyl]methyl]-N-methyl-carbamate, BzL-3h (616.35 mg, 1.09 mmol, 1 eq) in dioxane (3 mL) and H₂O (0.5 mL) was added Pd(dppf)C₁₂ (80.02 mg, 109.36 μmol, 0.1 eq) and Na₂CO₃ (231.81 mg, 2.19 mmol, 2 eq) in one portion at 25° C. under N₂. The mixture was stirred at 90° C. for 2 hrs. The mixture was filtered and concentrated. The residue was poured into H₂O (20 mL) and extracted with ethyl acetate (20 mL×2). The combined organic phase was washed with brine (20 mL), dried with anhydrous Na₂SO₄, filtered and concentrated in vacuum. The residue was purified by flash silica gel chromatography (ISCO®; 1 g SepaFlash® Silica Flash Column, Eluent of 0˜100% Ethyl acetate/Petroleum ether gradient at 75 mL/min) to obtain BzL-3i (360 mg, 468.69 μmol, 42.86% yield) was obtained as yellow solid.

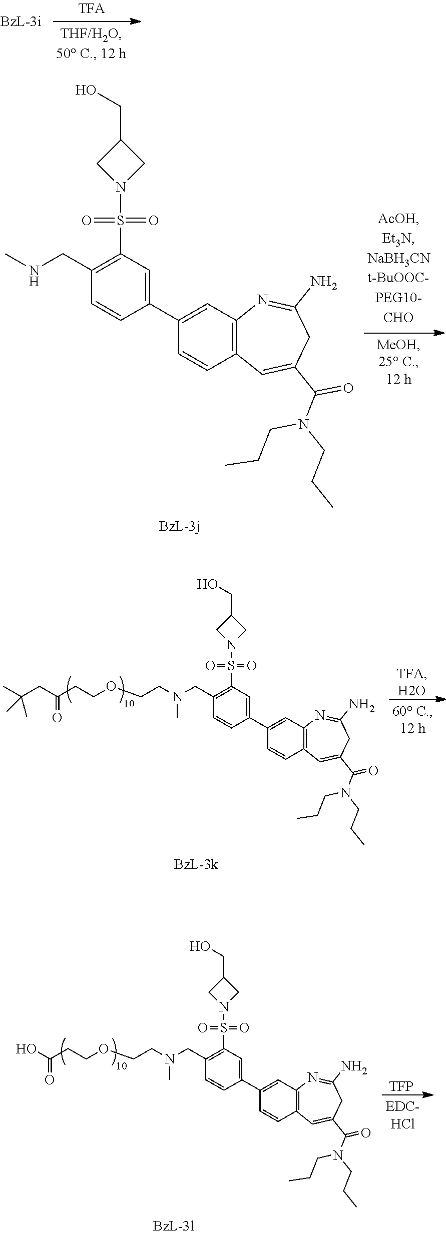

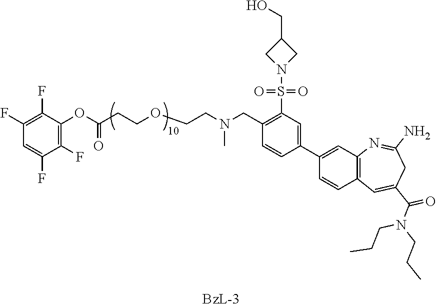

### Synthesis of 2-amino-8-[3-[3-(hydroxymethyl)azetidin-1-yl]sulfonyl-4-(methylamineomethyl)phenyl]-N,N-dipropyl-3H-1-benzazepine-4-carboxamide, BzL-3j

A mixture of tert-butyl N-[[4-[2-amino-4-(dipropylcarbamoyl)-3H-1-benzazepin-8-yl]-2-[3-[[tert-butyl(dimethyl)silyl]oxymethyl]azetidin-1-yl]sulfonyl-phenyl]methyl]-N-methyl-carbamate, BzL-3i (170 mg, 221.33 μmol, 1 eq) in THE (5 mL) and H₂O (1 mL) was added TFA (504.72 mg, 4.43 mmol, 327.74 μL, 20 eq) the mixture was stirred at 50° C. for 12 hrs. LC-MS showed reactant 1 was consumed completely and one main peak with desired mass was detected. The reaction mixture was filtered, and the filtrate was concentrated under reduced. The residue was purified by prep-HPLC (column: Nano-micro Kromasil C18 100×30 mm 5 um; mobile phase: [water(0.1% TFA)-ACN]; B %: 20%-45%, 10 min) to give BzL-3j (95 mg crude) product as a yellow solid. ¹H NMR (DMSO-d₆, 400 MHz) δ 12.49 (s, 1H), 9.88 (s, 1H), 9.50 (s, 1H), 8.87 (s, 2H), 8.24-8.22 (m, 1H), 8.17-8.16 (m, 1H), 7.92-7.90 (m, 1H), 7.74-7.71 (m, 1H), 7.67-7.70 (m, 2H), 7.06 (s, 1H), 4.79 (s, 1H), 4.46 (s, 2H), 3.85 (t, J=8.0 Hz, 2H), 3.61 (t, J=4.0 Hz, 2H), 3.35 (s, 4H), 2.67 (s, 3H), 2.64-2.55 (m, 2H), 1.74-1.39 (m, 4H), 0.86-0.80 (m, 6H). LC/MS [M+H]554.28 (calculated); LC/MS [M+H]554.40 (observed).

### Synthesis of tert-butyl 3-[2-[2-[2-[2-[2-[2-[2-[2-[2-[2-[[4-[2-amino-4-(dipropylcarbamoyl)-3H-1-benzazepin-8-yl]-2-[3-(hydroxymethyl)azetidin-1-yl]sulfonyl-phenyl]methyl-methyl-amino]ethoxy]ethoxy]ethoxy]ethoxy]ethoxy]ethoxy]ethoxy]ethoxy]ethoxy]ethoxy]propanoate, BzL-3k

To a mixture of 2-amino-8-[3-[3-(hydroxymethyl)azetidin-1-yl]sulfonyl-4-(methylaminomethyl)phenyl]-N,N-dipropyl-3H-1-benzazepine-4-carboxamide, BzL-3j (0.05 g, 90.30 μmol, 1 eq) and tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate, t-BuOOC-PEG10-CHO (52.80 mg, 90.30 μmol, 1 eq) in MeOH (2 mL) was added Et₃N (27.41 mg, 270.90 μmol, 37.71 μL, 3 eq) and AcOH (5.42 mg, 90.30 μmol, 5.16 μL, 1 eq) and NaBH₃CN (14.19 mg, 225.75 μmol, 2.5 eq) at 25° C. The mixture was stirred for 12 hrs. The mixture was concentrated in vacuum to afford BzL-3k (100 mg crude) as yellow oil.

### Synthesis of 3-[2-[2-[2-[2-[2-[2-[2-[2-[2-[2-[[4-[2-amino-4-(dipropylcarbamoyl)-3H-1-benzazepin-8-yl]-2-[3-(hydroxymethyl)azetidin-1-yl]sulfonyl-phenyl]methyl-methyl-amino]ethoxy]ethoxy]ethoxy]ethoxy]ethoxy]ethoxy]ethoxy]ethoxy]ethoxy]ethoxy]propanoic acid, BzL-31

To a solution of BzL-3k (100 mg, 89.09 μmol, 1 eq) in H₂O (1 mL) was added TFA (203.18 mg, 1.78 mmol, 131.93 μL, 20 eq). The mixture was stirred at 60° C. for 12 hrs. The reaction mixture was filtered, and the filtrate was concentrated under reduced pressure to give a residue. The residue was purified by prep-HPLC (column: Luna C18 100×30 5 u; liquid phase: [A-TFA/H₂O=0.075% v/v; B-ACN], B %: 20%-45%, 10 min]) to obtain BzL-31 (20 mg, 18.38 μmol, 20.63% yield, 97.989% purity) as colorless oil. ¹H NMR (MeOD, 400 MHz) δ 8.39-8.38 (m, 1H), 8.23-8.20 (m, 1H), 7.98-7.96 (m, 1H), 7.83-7.81 (m, 2H), 7.73-7.71 (m, 1H), 7.11 (s, 1H), 4.02-4.00 (m, 2H), 3.94-3.88 (m, 2H), 3.79-3.74 (m, 2H), 3.74-3.40 (m, 45H), 3.40-3.35 (m, 2H), 2.98-2.94 (m, 3H), 2.79-2.71 (m, 2H), 2.56-2.51 (m, 2H), 1.80-1.66 (m, 5H), 0.95 (s, 6H). LC/MS [M+2H/2]533.78 (calculated); LC/MS [M+2H/2]534.20 (observed).

2,3,5,6-Tetrafluorophenyl 1-(4-(2-amino-4-(dipropylcarbamoyl)-3H-benzo[b]azepin-8-yl)-2-((3-(hydroxymethyl)azetidin-1-yl)sulfonyl)phenyl)-2-methyl-5,8,11,14,17,20,23,26,29,32-decaoxa-2-azapentatriacontan-35-oate, BzL-3 was synthesized according to the procedure described for BzL-22. LC/MS [M+H]1214.56 (calculated); LC/MS [M+H]1214.97 (observed).

### Example 34 Synthesis of BzL-4

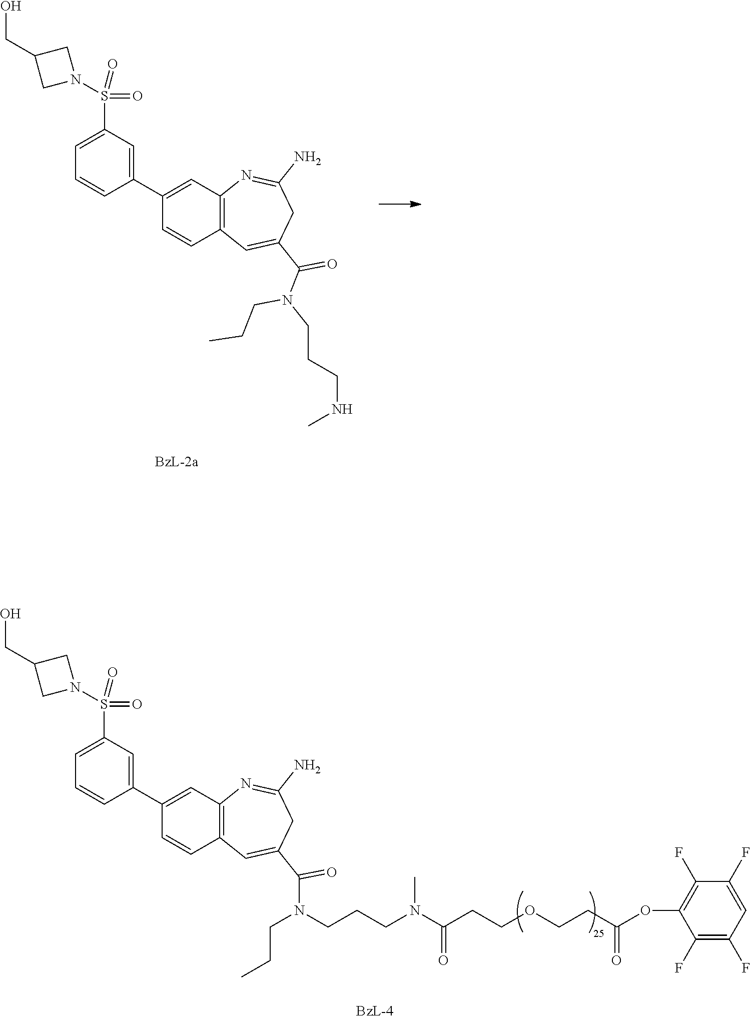

2,3,5,6-Tetrafluorophenyl 84-(2-amino-8-(3-((3-(hydroxymethyl)azetidin-1-yl)sulfonyl)phenyl)-3H-benzo[b]azepine-4-carbonyl)-80-methyl-79-oxo-4,7,10,13,16,19,22,25,28,31,34,37,40,43,46,49,52,55,58,61,64,67,70,73,76-pentacosaoxa-80,84-diazaheptaoctacontanoate, BzL-4 was synthesized according to the procedure described for BzL-15. LC/MS [M+H]1888.93 (calculated); LC/MS [M+H]1889.53 (observed).

### Example 35 Synthesis of BzL-5

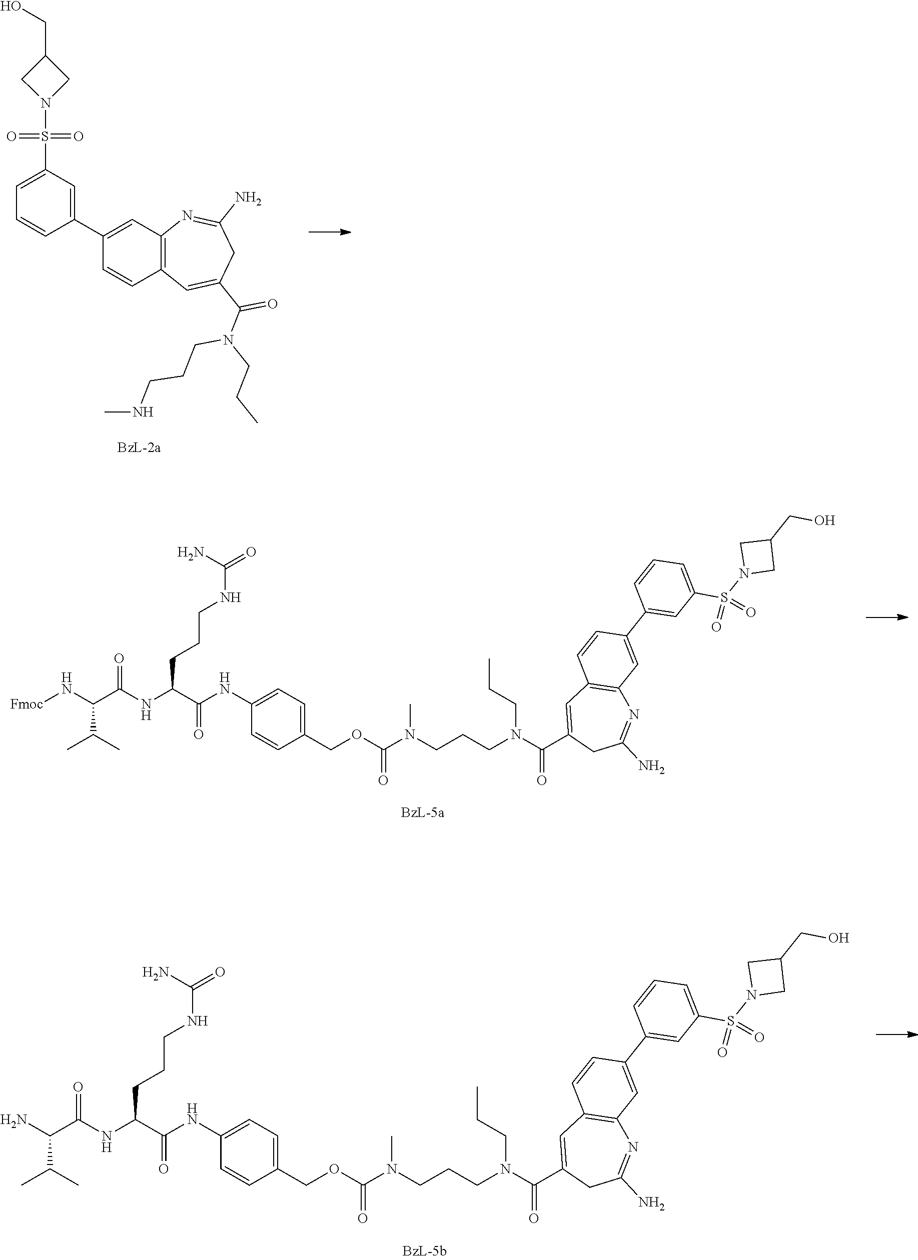

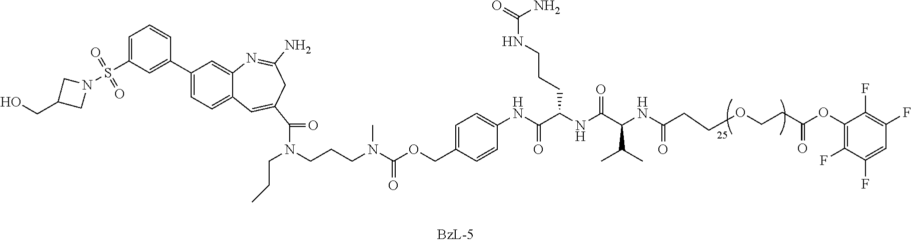

4-((S)-2-((S)-2-((((9H-Fluoren-9-yl)methoxy)carbonyl)amino)-3-methylbutanamido)-5-ureidopentanamido)benzyl (3-(2-amino-8-(3-((3-(hydroxymethyl)azetidin-1-yl)sulfonyl)phenyl)-N-propyl-3H-benzo[b]azepine-4-carboxamido)propyl)(methyl)carbamate, BzL-5a was synthesized according to the procedure described for BzL-26a.

4-((S)-2-((S)-2-Amino-3-methylbutanamido)-5-ureidopentanamido)benzyl (3-(2-amino-8-(3-((3-(hydroxymethyl)azetidin-1-yl)sulfonyl)phenyl)-N-propyl-3H-benzo[b]azepine-4-carboxamido)propyl)(methyl)carbamate, BzL-5b was synthesized according to the procedure described for BzL-26. LC/MS [M+H]945.47 (calculated); LC/MS [M+H]945.82 (observed).

2,3,5,6-Tetrafluorophenyl (6S,9S)-1-amino-6-((4-((((3-(2-amino-8-(3-((3-(hydroxymethyl)azetidin-1-yl)sulfonyl)phenyl)-N-propyl-3H-benzo[b]azepine-4-carboxamido)propyl)(methyl)carbamoyl)oxy)methyl)phenyl)carbamoyl)-9-isopropyl-1,8,11-trioxo-14,17,20,23,26,29,32,35,38,41,44,47,50,53,56,59,62,65,68,71,74,77,80,83,86-pentacosaoxa-2,7,10-triazanonaoctacontan-89-oate, BzL-5 was synthesized according to the procedure described for BzL-15. LC/MS [M+2H/2]1147.57 (calculated); LC/MS [M+H]1148.37 (observed).

### Example 36 Synthesis of BzL-13

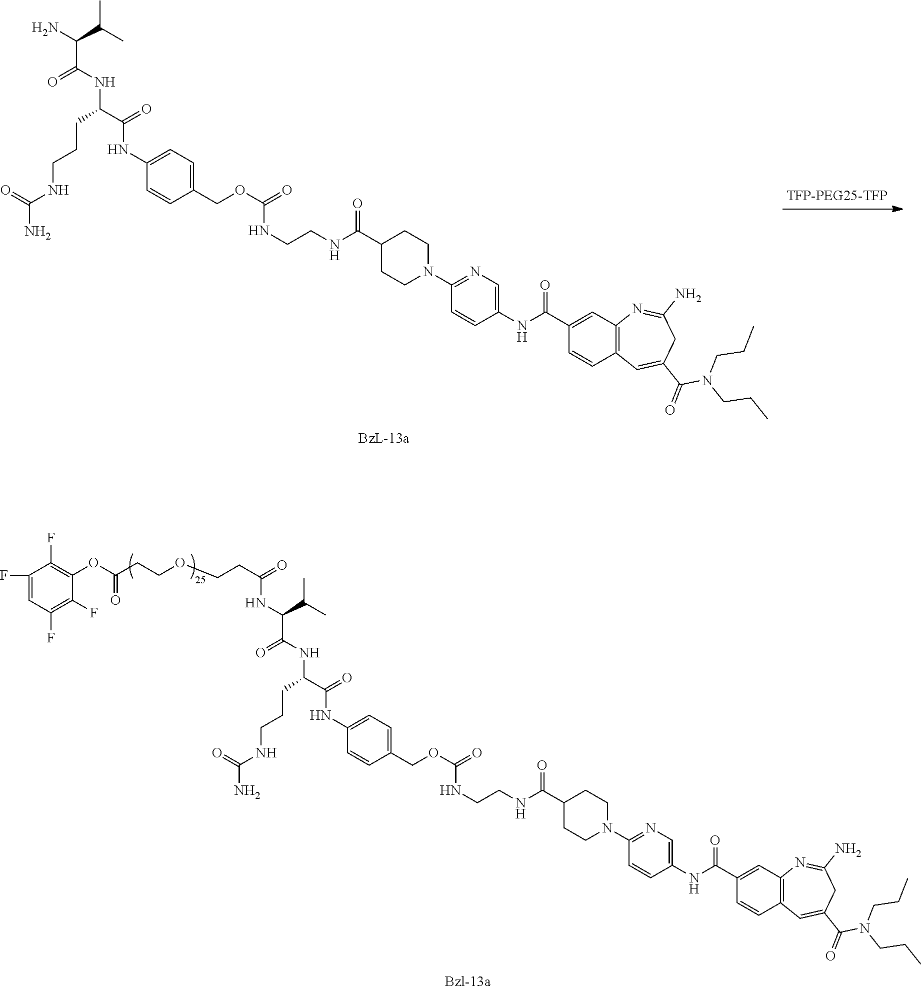

2,3,5,6-Tetrafluorophenyl (6S,9S)-1-amino-6-((4-((((2-(1-(5-(2-amino-4-(dipropylcarbamoyl)-3H-benzo[b]azepine-8-carboxamido)pyridin-2-yl)piperidine-4-carboxamido)ethyl)carbamoyl)oxy)methyl)phenyl)carbamoyl)-9-isopropyl-1,8,11-trioxo-14,17,20,23,26,29,32,35,38,41,44,47,50,53,56,59,62,65,68,71,74,77,80,83,86-pentacosaoxa-2,7,10-triazanonaoctacontan-89-oate, BzL-13 was synthesized from BzL-13a and TFP-PEG25-TFP according to the procedure described for BzL-15. LC/MS [M+2H/2]1165.10 (calculated); LC/MS [M+H]1165.91 (observed).

### Example 37 Synthesis of BzL-14

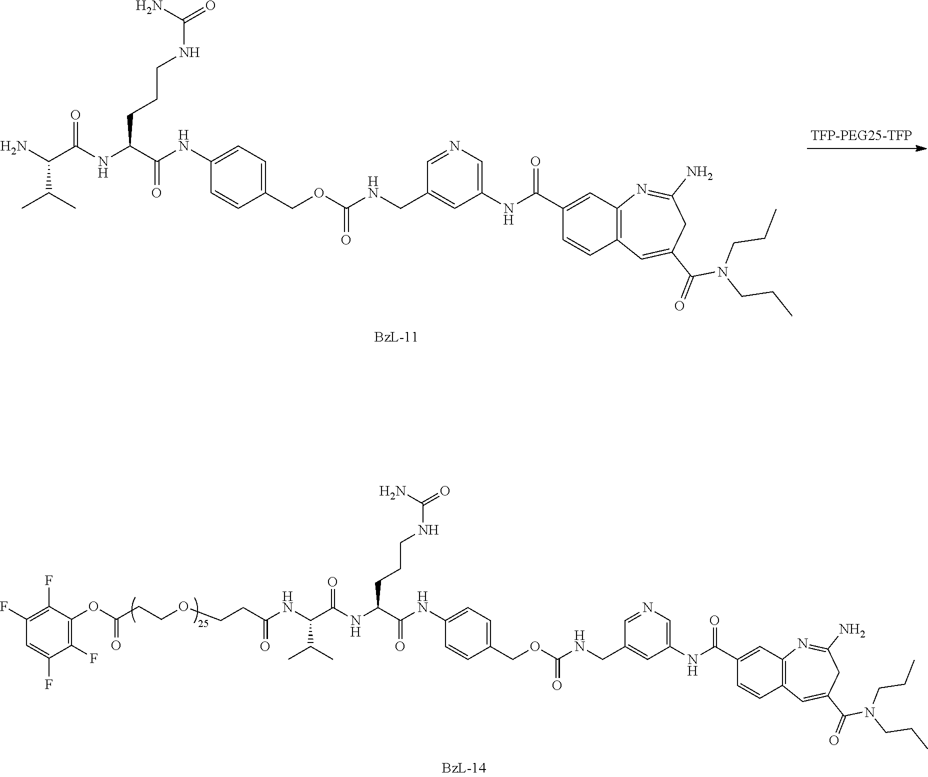

2,3,5,6-Tetrafluorophenyl (6S,9S)-1-amino-6-((4-(((((6-(2-amino-4-(dipropylcarbamoyl)-3H-benzo[b]azepine-8-carboxamido)pyridin-3-yl)methyl)carbamoyl)oxy)methyl)phenyl)carbamoyl)-9-isopropyl-1,8,11-trioxo-14,17,20,23,26,29,32,35,38,41,44,47,50,53,56,59,62,65,68,71,74,77,80,83,86-pentacosaoxa-2,7,10-triazanonaoctacontan-89-oate, BzL-14 was synthesized from BzL-11 and TFP-PEG25-TFP according to the procedure described for BzL-15. LC/MS [M+2H/2]1095.06 (calculated); LC/MS [M+H]1095.87 (observed).

### Example 38 Synthesis of BzL-15

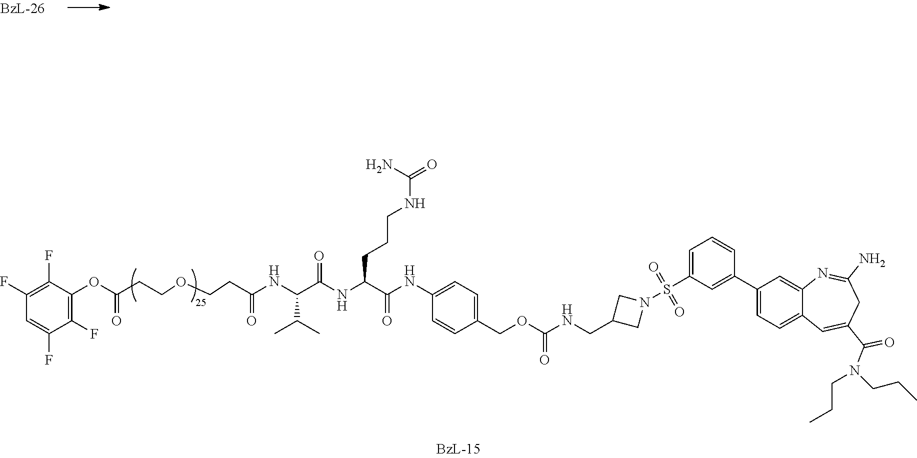

### Synthesis of 2,3,5,6-tetrafluorophenyl (6S,9S)-1-amino-6-((4-(((((1-((3-(2-amino-4-(dipropylcarbamoyl)-3H-benzo[b]azepin-8-yl)phenyl)sulfonyl)azetidin-3-yl)methyl)carbamoyl)oxy)methyl)phenyl)carbamoyl)-9-isopropyl-1,8,11-trioxo-14,17,20,23,26,29,32,35,38,41,44,47,50,53,56,59,62,65,68,71,74,77,80,83,86-pentacosaoxa-2,7,10-triazanonaoctacontan-89-oate, BzL-15)

### Synthesis of bis(2,3,5,6-tetrafluorophenyl) 4,7,10,13,16,19,22,25,28,31,34,37,40,43,46,49,52,55,58,61,64,67,70,73,76-pentacosaoxanonaheptacontanedioate, TFP-PEG25-TFP

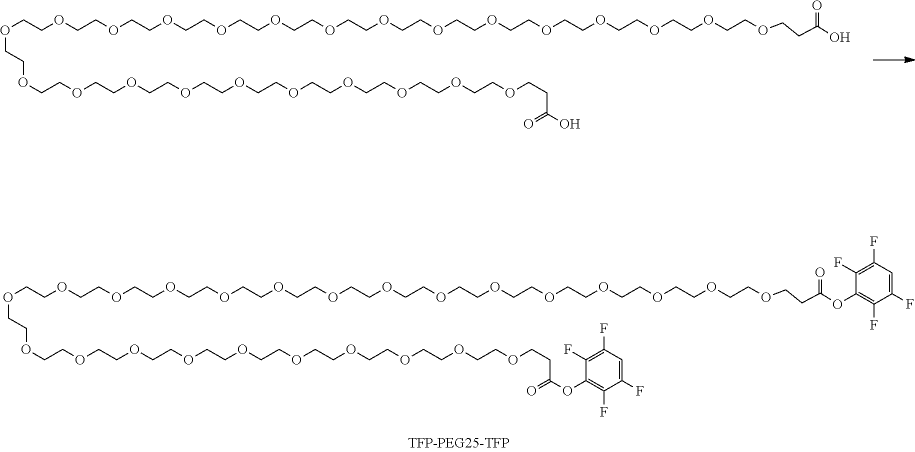 [SMILES: O=C(O)CCOCCOCCOCCOCCOCCOCCOCCOCCOCCOCCOCCOCCOCCOCCOCCOCCOCCOCCOCCOCCOCCOCCOCCOCCOCCC(=O)O ; O=C(CCOCCOCCOCCOCCOCCOCCOCCOCCOCCOCCOCCOCCOCCOCCOCCOCCOCCOCCOCCOCCOCCOCCOCCOCCOCCC(=O)Oc1c(F)c(F)cc(F)c1F)Oc1c(F)c(F)cc(F)c1F]

A vial was charged with 4,7,10,13,16,19,22,25,28,31,34,37,40,43,46,49,52,55,58,61,64,67,70,73,76-pentacosaoxanonaheptacontanedioic acid (269 mg, 0.221 mmol), 2,3,5,6-tetrafluorophenol (110 mg, 0.662 mmol), collidine (176 μL, 1.33 mmol), 1-ethyl-3-(3-dimethylaminopropyl)carbodiimide (127 mg, 0.221 mmol) and 3 mL DMF. The reaction was stirred for 16 h, then purified by reverse phase preparative HPLC utilizing a 25-75% gradient of acetonitrile:water containing 0.1% trifluoroacetic acid. The purified fractions were combined and lyophilized to afford 266 mg of TFP-PEG25-TFP in 79% yield. LC/MS [M+H]1515.68 (calculated); LC/MS [M+H]1516.00 (observed).

A vial was charged with BzL-26 (11.9 mg, 0.013 mmol), TFP-PEG25-TFP (19.7 mg, 0.013 mmol), collidine (5.6 μL, 0.042 mmol) in 300 μL DMF. The reaction was maintained for 5 h and then purified by reverse phase preparative HPLC utilizing a 25-75% gradient of acetonitrile:water containing 0.1% trifluoroacetic acid. The purified fractions were combined and lyophilized to afford 7.7 mg of BzL-15 in 26% yield. LC/MS [M+2H/2]1132.56 (calculated); LC/MS [M+2H/2]1133.30 (observed).

### Example 39 Synthesis of BzL-16

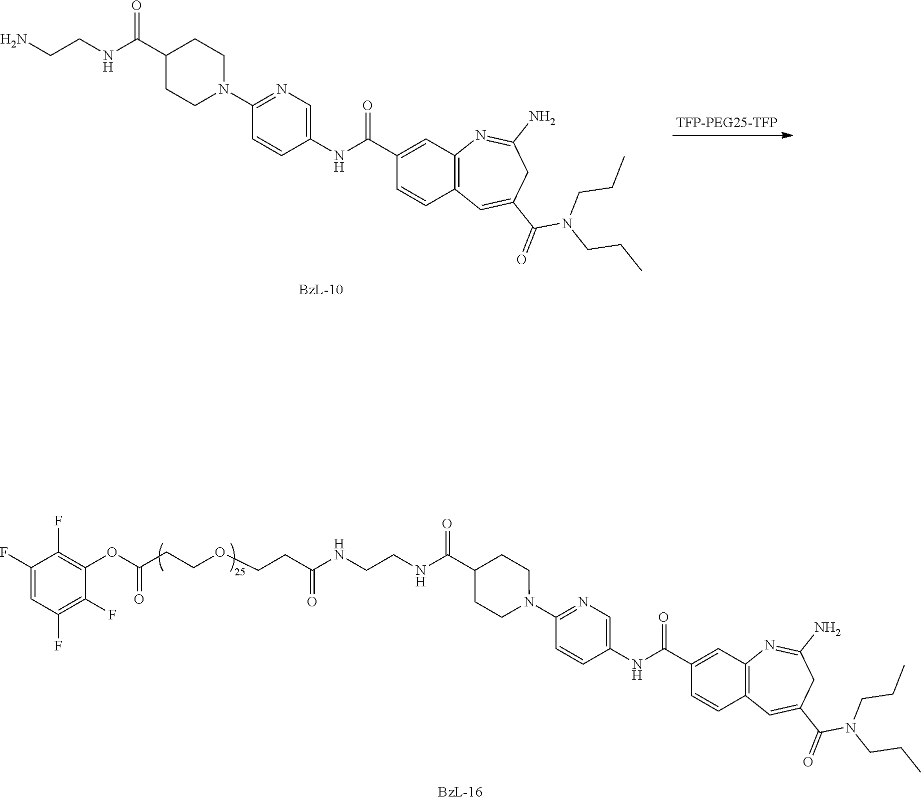

Synthesis of 2,3,5,6-tetrafluorophenyl 1-(1-(5-(2-amino-4-(dipropylcarbamoyl)-3H-benzo[b]azepine-8-carboxamido)pyridin-2-yl)piperidin-4-yl)-1,6-dioxo-9,12,15,18,21,24,27,30,33,36,39,42,45,48,51,54,57,60,63,66,69,72,75,78,81-pentacosaoxa-2,5-diazatetraoctacontan-84-oate, BzL-16 was synthesized from BzL-10 and TFP-PEG25-TFP according to the procedure described for Bz-31. LC/MS [M+H]1924.01 (calculated); LC/MS [M+H]1925.23 (observed).

### Example 40 Synthesis of BzL-17

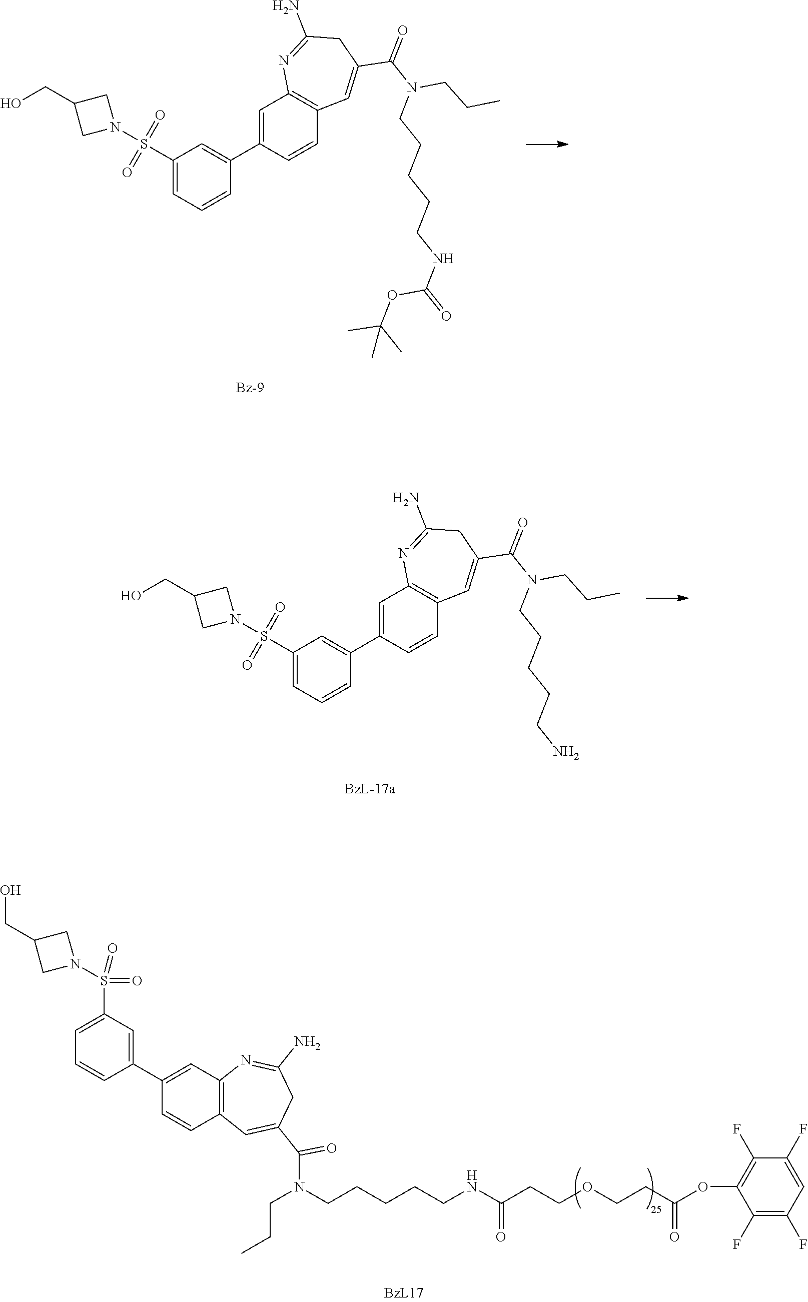

Synthesis of 2-amino-N-(5-aminopentyl)-8-(3-((3-(hydroxymethyl)azetidin-1-yl)sulfonyl)phenyl)-N-propyl-3H-benzo[b]azepine-4-carboxamide, BzL-17a. A vial was charged with Bz-9 (28 mg, 0.043 mmol), 300 μL DCM and 100 μL trifluoroacetic acid. The reaction was maintained for 1 h, upon which it was concentrated under reduced pressure. The resultant oil was azeotroped thrice with 1 mL toluene, after which was added 1 mL methanol and K₂CO₃ (38 mg, 0.28 mmol). After stirring for 16 h, the reaction was filtered and concentrated under reduced pressure and then purified by reverse phase preparative HPLC utilizing a 25-75% gradient of acetonitrile:water containing 0.1% trifluoracetic acid. The purified fractions were combined and lyophilized to afford 5.8 mg of BzL-17a in 24% yield. LC/MS [M+H]554.28 (calculated); LC/MS [M+H]554.47 (observed).

Synthesis of 2,3,5,6-tetrafluorophenyl 86-(2-amino-8-(3-((3-(hydroxymethyl)azetidin-1-yl)sulfonyl)phenyl)-3H-benzo[b]azepine-4-carbonyl)-79-oxo-4,7,10,13,16,19,22,25,28,31,34,37,40,43,46,49,52,55,58,61,64,67,70,73,76-pentacosaoxa-80,86-diazanonaoctacontanoate, BzL-17. A vial was charged with BzL-17a (5.8 mg, 0.011 mmol), TFP-PEG25-TFP (23.8 mg, 0.016 mmol), collidine (5.6 μL, 0.042 mmol) in 300 μL DMF. The reaction was maintained for 5 h and then purified by reverse phase preparative HPLC utilizing a 25-75% gradient of acetonitrile:water (ACN:H2O) containing 0.1% trifluoroacetic acid (TFA). The purified fractions were combined and lyophilized to afford 5.0 mg of BzL-17 in 25% yield. LC/MS [M+H]1902.95 (calculated); LC/MS [M+H]1903.37 (observed).

### Example 41 Synthesis of BzL-18

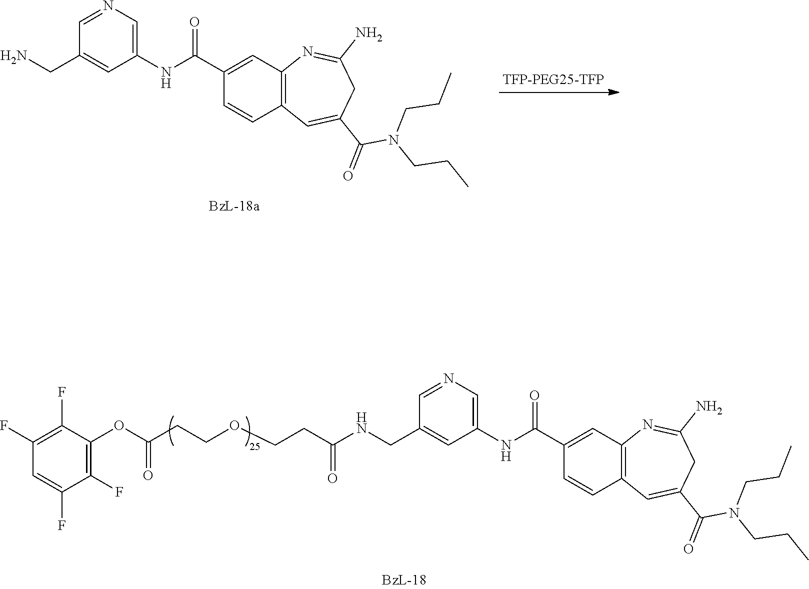

2,3,5,6-Tetrafluorophenyl 1-(6-(2-amino-4-(dipropylcarbamoyl)-3H-benzo[b]azepine-8-carboxamido)pyridin-3-yl)-3-oxo-6,9,12,15,18,21,24,27,30,33,36,39,42,45,48,51,54,57,60,63,66,69,72,75,78-pentacosaoxa-2-azahenoctacontan-81-oate, BzL-18 was synthesized from BzL-18a and TFP-PEG25-TFP according to the procedure described for BzL-15. LC/MS [M+H]1783.92 (calculated); LC/MS [M+H]1784.19 (observed).

### Example 42 Synthesis of BzL-19

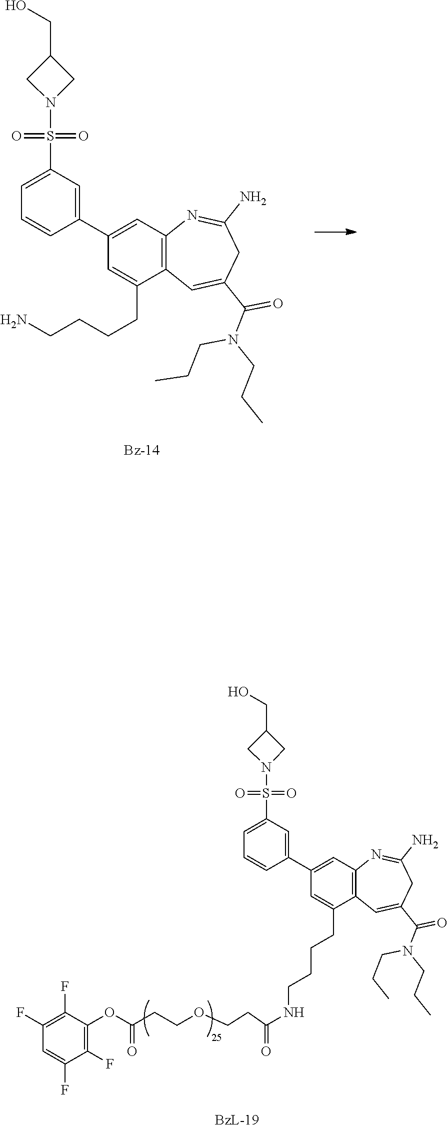

2,3,5,6-Tetrafluorophenyl 84-(2-amino-4-(dipropylcarbamoyl)-8-(3-((3-(hydroxymethyl)azetidin-1-yl)sulfonyl)phenyl)-3H-benzo[b]azepin-6-yl)-79-oxo-4,7,10,13,16,19,22,25,28,31,34,37,40,43,46,49,52,55,58,61,64,67,70,73,76-pentacosaoxa-80-azatetraoctacontanoate, BzL-19 was synthesized from Bz-14 and TFP-PEG25-TFP according to the procedure described for BzL-15. LC/MS [M+H]1930.98 (calculated); LC/MS [M+H]1931.24 (observed).

### Example 43 Synthesis of BzL-20

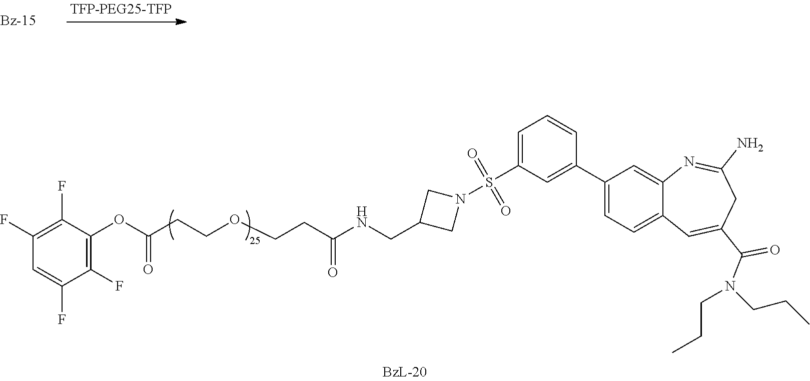

2,3,5,6-Tetrafluorophenyl 1-(1-((3-(2-amino-4-(dipropylcarbamoyl)-3H-benzo[b]azepin-8-yl)phenyl)sulfonyl)azetidin-3-yl)-3-oxo-6,9,12,15,18,21,24,27,30,33,36,39,42,45,48,51,54,57,60,63,66,69,72,75,78-pentacosaoxa-2-azahenoctacontan-81-oate, BzL-20 was synthesized from reaction of TFP-PEG25-TFP and Bz-15 according to the procedure described for BzL-15. LC/MS [M+H]1858.92 (calculated); LC/MS [M+H]1859.59 (observed).

### Example 44 Synthesis of BzL-21

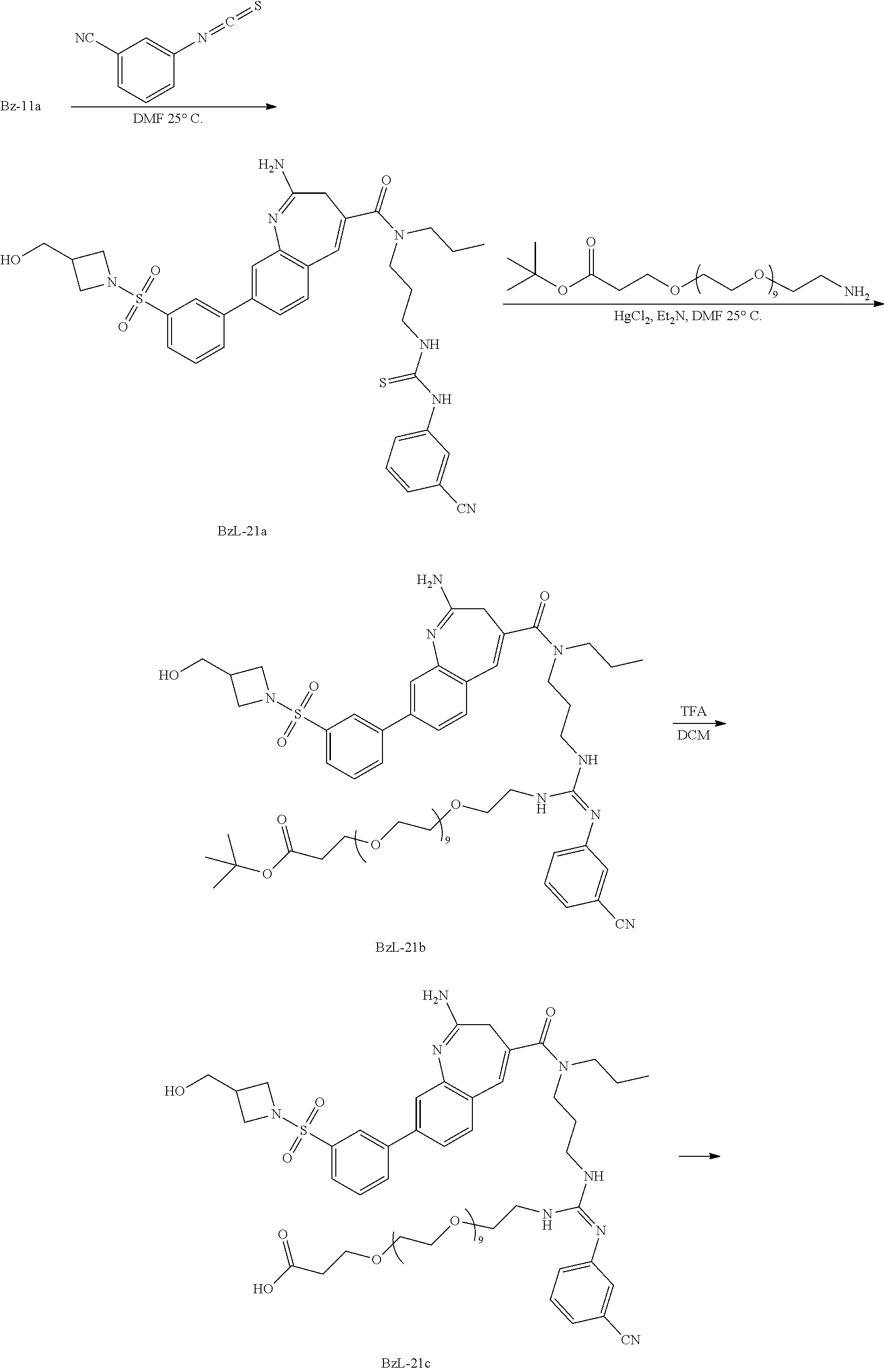

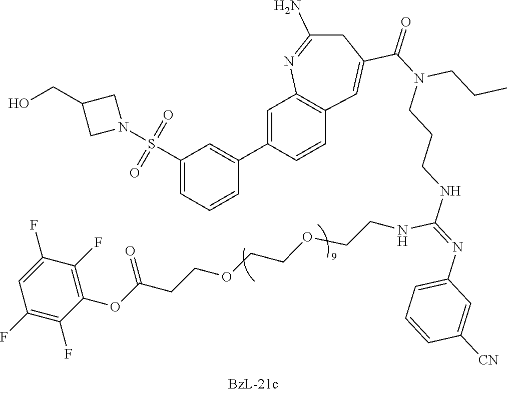

### Synthesis of 2-amino-N-[3-[(3-cyanophenyl)carbamothioylamino]propyl]-8-[3-[3-(hydroxymethyl)azetidin-1-yl]sulfonylphenyl]-N-propyl-3H-1-benzazepine-4-carboxamide, BzL-21a

To a mixture of 2-amino-N-(3-aminopropyl)-8-[3-[3-(hydroxymethyl)azetidin-1-yl]sulfonylphenyl]-N-propyl-3H-1-benzazepine-4-carboxamide, Bz-11a (0.1 g, 190.24 μmol, 1 eq) in DMF (2 mL) was added 3-isothiocyanatobenzonitrile (30.48 mg, 190.24 μmol, 1 eq) in one portion at 15° C. The mixture was stirred at 15° C. for 3 hours. LCMS showed the desired was detected. The mixture was filtered and purified by prep-HPLC (column: Nano-micro Kromasil C18 100×30 mm, Sum; mobile phase: [water (0.1% TFA)-ACN]; B %: 20%-60%, 10 min) to give 2-amino-N-[3-[(3-cyanophenyl)carbamothioylamino]propyl]-8-[3-[3-(hydroxymethyl)azetidin-1-yl]sulfonylphenyl]-N-propyl-3H-1-benzazepine-4-carboxamide, BzL-21a (0.06 g, 87.48 μmol, 45.99% yield) was obtained as light yellow solid.

### Synthesis of tert-butyl 3-[2-[2-[2-[2-[2-[2-[2-[2-[2-[2-[(Z)-[[3-[[2-amino-8-[3-[3- (hydroxymethyl)azetidin-1-yl]sulfonylphenyl]-3H-1-benzazepine-4-carbonyl]-propyl-amino]propylamino]-(3-cyanoanilino)methylene]amino]ethoxy]ethoxy]ethoxy]ethoxy]ethoxy]ethoxy]ethoxy]ethoxy]eth oxy]ethoxy]propanoate, BzL-21b

To a mixture of BzL-21a (0.06 g, 87.48 μmol, 1 eq) and tert-butyl 3-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]propanoate (61.49 mg, 104.98 μmol, 1.2 eq) in DMF (2 mL) was added Et₃N (17.70 mg, 174.96 μmol, 24.35 μL, 2 eq) and HgCl₂ (28.50 mg, 104.98 μmol, 5.24 μL, 1.2 eq). The mixture was stirred at 15° C. for 18 hours. LCMS showed the reactant was consumed. The mixture was filtered and poured into water (10 mL). The aqueous phase was extracted with ethyl acetate (10 mL×3). The combined organic phase was concentrated to give BzL-21b (0.1 g, crude) as light yellow oil which was used into the next step without further purification.

### Synthesis of 3-[2-[2-[2-[2-[2-[2-[2-[2-[2-[2-[(Z)-[[3-[[2-amino-8-[3-[3- (hydroxymethyl)azetidin-1-yl]sulfonylphenyl]-3H-1-benzazepine-4-carbonyl]-propyl-amino]propylamino]-(3-cyanoanilino)methylene]amino]ethoxy]ethoxy]ethoxy]ethoxy]ethoxy]ethoxy]ethoxy]ethoxy]eth oxy]ethoxy]propanoic acid, BzL-21c

To a mixture of BzL-21b (86.04 mg, 69.52 μmol, 1 eq) in H₂O (10 mL) was added TFA (396.36 mg, 3.48 mmol, 257.38 μL, 50 eq) in one portion at 15° C. The mixture was stirred at 85° C. for 10 min. LCMS showed the reactant was consumed. The mixture was concentrated. The residue was purified by prep-HPLC (column: Nano-micro Kromasil C18 100×30 mm, Sum; mobile phase: [water (0.10% TFA)-ACN]; B %: 10%-40%, 10 min) to give BzL-21c (18 mg, 13.71 μmol, 19.72% yield, 90% purity) was obtained as a white solid. ¹H NMR (MeOD, 400 MHz) δ 8.12-8.08 (m, 2H), 7.92 (d, J=8.0 Hz, 1H), 7.84-7.81 (m, 4H), 7.64 (s, 3H), 7.12 (s, 1H), 3.87 (t, J=8.4 Hz, 2H), 3.72-3.70 (m, 9H), 3.63-3.58 (m, 38H), 3.43-3.41 (m, 6H), 2.62-2.57 (m, 1H), 2.52 (t, J=6.0 Hz, 2H), 2.04 (s, 2H), 1.75-1.70 (m, 3H), 0.96-0.92 (m, 3H).

2,3,5,6-Tetrafluorophenyl (Z)-40-(2-amino-8-(3-((3-(hydroxymethyl)azetidin-1-yl)sulfonyl)phenyl)-3H-benzo[b]azepine-4-carbonyl)-35-((3-cyanophenyl)imino)-4,7,10,13,16,19,22,25,28,31-decaoxa-34,36,40-triazatritetracontanoate, BzL-21 was synthesized according to the procedure described for BzL-22. LC/MS [M+H]1329.57 (calculated); LC/MS [M+H]1329.77 (observed).

### Example 45 Synthesis of BzL-22

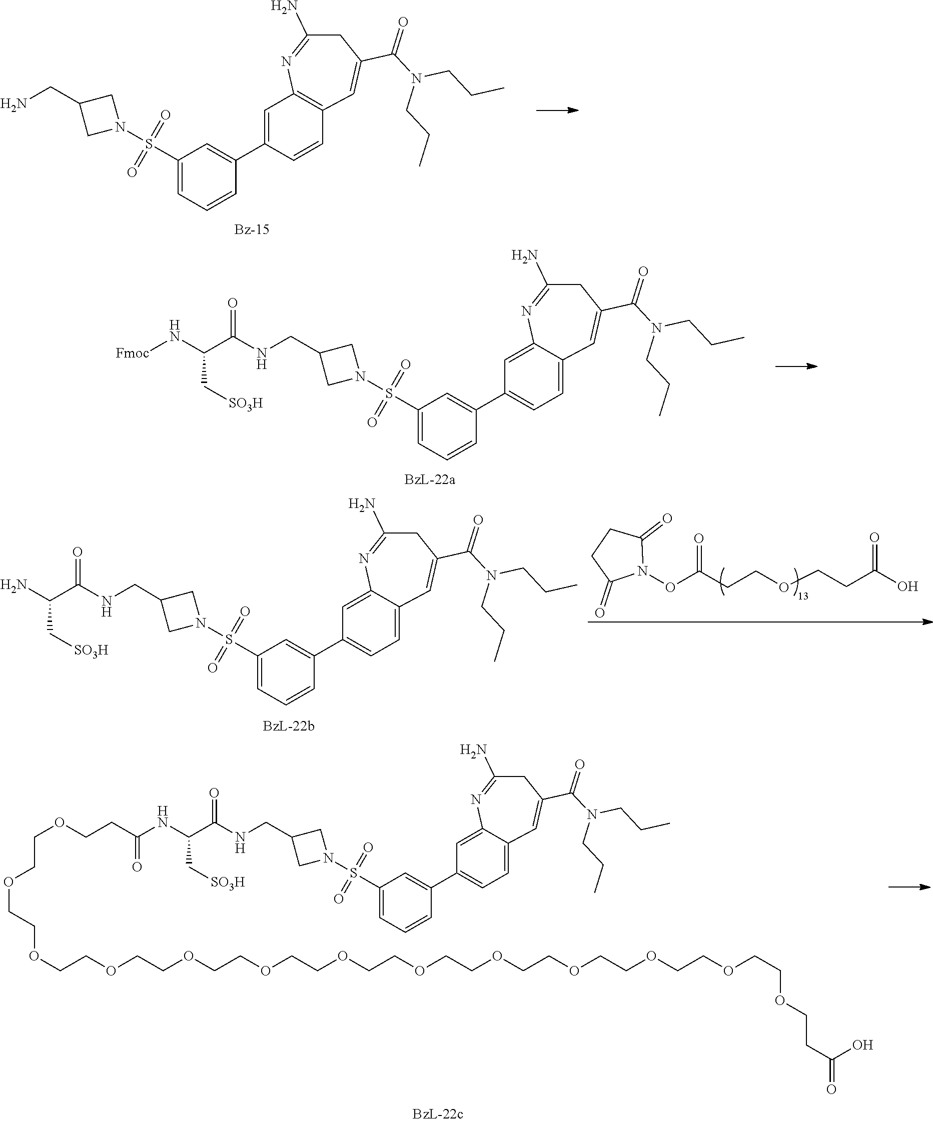

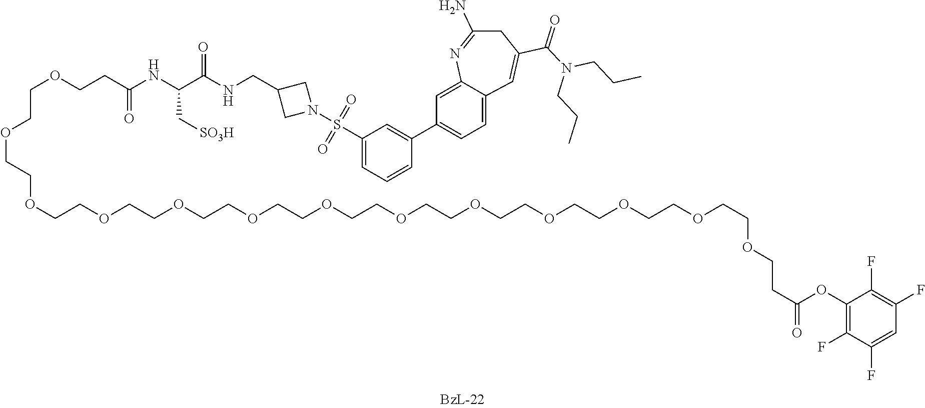 [SMILES: CCCN(CCC)C(=O)C1=Cc2ccc(-c3cccc(S(=O)(=O)N4CC(CNC(=O)[C@H](CS(=O)(=O)O)NC(=O)CCOCCOCCOCCOCCOCCOCCOCCOCCOCCOCCOCCOCCOCCC(=O)Oc5c(F)c(F)cc(F)c5F)C4)c3)cc2N=C(N)C1]

### Synthesis of (R)-2-((((9H-Fluoren-9-yl)methoxy)carbonyl)amino)-3-(((1-((3-(2-amino-4-(dipropylcarbamoyl)-3H-benzo[b]azepin-8-yl)phenyl)sulfonyl)azetidin-3-yl)methyl)amino)-3-oxopropane-1-sulfonic acid, BzL-22a

A vial was charged with Bz-15 (14.7 mg, 0.024 mmol), Fmoc-L-Cysteic Acid (11.2 mg, 0.024 mmol), collidine (12 μL, 0.090 mmol), HATU (12 mg, 0.032 mmol) and 500 μL DMF. The reaction was stirred until Bz-15 was consumed by LCMS. The crude mixture was purified by reverse phase preparative HPLC utilizing a 25-75% gradient of acetonitrile:water containing 0.1% trifluoroacetic acid. The purified fractions were combined and lyophilized to afford 8.6 mg of BzL-22a in 41% yield. LC/MS [M+H]883.32 (calculated); LC/MS [M+H]883.49 (observed).

### Synthesis of (R)-2-amino-3-(((1-((3-(2-amino-4-(dipropylcarbamoyl)-3H-benzo[b]azepin-8-yl)phenyl)sulfonyl)azetidin-3-yl)methyl)amino)-3-oxopropane-1-sulfonic acid, BzL-22b

A vial was charged with BzL-22a (8.6 mg, 0.01 mmol), diethylamine (10 μL, 0.10 mmol), 100 μL acetonitrile and 50 μL DMF. The reaction was stirred for 3 h, then concentrated under reduced pressure. The crude reaction was azeotroped thrice with 2 mL toluene and take on to the subsequent step.

Synthesis of (R)-1-(1-((3-(2-amino-4-(dipropylcarbamoyl)-3H-benzo[b]azepin-8-yl)phenyl)sulfonyl)azetidin-3-yl)-3,6-dioxo-4-(sulfomethyl)-9,12,15,18,21,24,27,30,33,36,39,42,45-tridecaoxa-2,5-diazaoctatetracontan-48-oic acid, BzL-22c A vial was charged with crude BzL-22b (0.01 mmol), 43-((2,5-dioxopyrrolidin-1-yl)oxy)-43-oxo-4,7,10,13,16,19,22,25,28,31,34,37,40-tridecaoxatritetracontanoic acid (7.7 mg, 0.01 mmol), diisopropylethylamine (5.3 μL, 0.03 mmol), 1-hydroxy-7-azabenzotriazole, HOAt, CAS Reg. No. 39968-33-7 (4 mg, 0.03 mmol) and 140 μL DMF. The reaction was stirred for 8 h, then purified by reverse phase preparative HPLC utilizing a 25-75% gradient of acetonitrile:water containing 0.1% trifluoroacetic acid. The purified fractions were combined and lyophilized to afford 8.4 mg of BzL-22c in 64% yield. LC/MS [M+H]1333.60 (calculated); LC/MS [M+H]1333.69 (observed).

Synthesis of (R)-2-(((1-((3-(2-amino-4-(dipropylcarbamoyl)-3H-benzo[b]azepin-8-yl)phenyl)sulfonyl)azetidin-3-yl)methyl)carbamoyl)-4,46-dioxo-46-(2,3,5,6-tetrafluorophenoxy)-7,10,13,16,19,22,25,28,31,34,37,40,43-tridecaoxa-3-azahexatetracontane-1-sulfonic acid, BzL-22.

A vial was charged with BzL-22c (7.2 mg, 0.005 mmol), 2,3,5,6-tetrafluorophenol (1.8 mg, 0.011 mmol), collidine (2.2 μL, 0.016 mmol), 1-ethyl-3-(3-dimethylaminopropyl)carbodiimide (1 mg, 0.005 mmol) and 100 μL DMF. The reaction was stirred for 16 h, then purified by reverse phase preparative HPLC utilizing a 25-75% gradient of acetonitrile:water containing 0.1% trifluoroacetic acid. The purified fractions were combined and lyophilized to afford 5.3 mg of BzL-22 in 66% yield. LC/MS [M+H]1481.60 (calculated); LC/MS [M+H]1481.82 (observed).

### Example 46 Synthesis of BzL-23

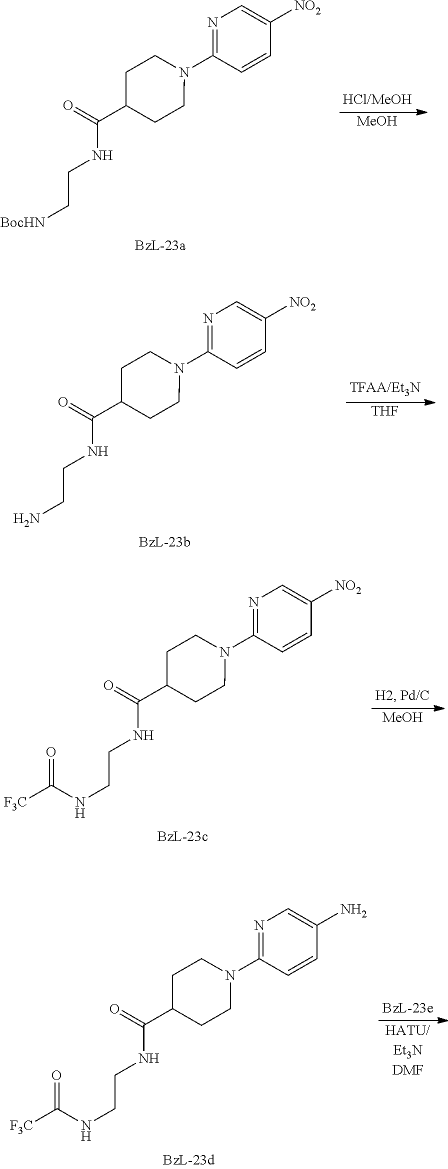

### Synthesis of N-(2-aminoethyl)-1-(5-nitropyridin-2-yl)piperidine-4-carboxamide, BzL-23b

To a mixture of tert-butyl N-[2-[[1-(5-nitro-2-pyridyl)piperidine-4-carbonyl]amino]ethyl]carbamate, BzL-23a (0.5 g, 1.27 mmol, 1 eq) in EtOAc (10 mL) was added HCl/EtOAc (4 M, 3.18 mL, 10 eq) at 25° C. The mixture was stirred at 25° C. for 2 hours. LCMS showed the reaction was completed. The reaction was concentrated in vacuum to give BzL-23b (0.4 g, 1.21 mmol, 95.44% yield, HCl) as a yellow solid.

### Synthesis of 1-(5-nitropyridin-2-yl)-N-(2-(2,2,2-trifluoroacetamido) ethyl)piperidine-4-carboxamide, BzL-23c

To a mixture of N-(2-aminoethyl)-1-(5-nitro-2-pyridyl)piperidine-4-carboxamide, BzL-23b (0.4 g, 1.21 mmol, 1 eq, HCl) in THE (10 mL) was added Et₃N (368.21 mg, 3.64 mmol, 506.47 μL, 3 eq) and (2,2,2-trifluoroacetyl) 2,2,2-trifluoroacetate (382.13 mg, 1.82 mmol, 253.06 μL, 1.5 eq) at 25° C. The mixture was stirred at 25° C. for 1 hours. LCMS showed major as desired. The mixture was poured into water (50 mL). The aqueous phase was extracted with ethyl acetate (30 mL×3). The combined organic phase was washed with brine (30 mL), dried with anhydrous Na₂SO₄, filtered and concentrated in vacuum. The residue was used to next step directly, containing BzL-23c (0.4 g, 1.03 mmol, 84.71% yield) as a yellow solid. ¹H NMR (DMSO-d₆, 400 MHz) δ 9.37-9.45 (m, 1H), 8.95 (d, J=2.8 Hz, 1H), 8.19 (dd, J=9.6, 2.8 Hz, 1H), 8.03 (br t, J=5.2 Hz, 1H), 6.96 (d, J=9.6 Hz, 1H), 4.47-4.53 (m, 2H), 2.99-3.25 (m, 6H), 2.38-2.47 (m, 3H), 1.73-1.80 (m, 2H), 1.41-1.58 (m, 2H)

### Synthesis of 1-(5-aminopyridin-2-yl)-N-(2-(2,2,2-trifluoroacetamido) ethyl)piperidine-4-carboxamide, BzL-23d

To a solution of 1-(5-nitro-2-pyridyl)-N-[2-[(2,2,2-trifluoroacetyl)amino]ethyl]piperidine-4-carboxamide, BzL-23c (0.4 g, 1.03 mmol, 1 eq) in MeOH (30 mL) was added Pd/C (0.5 g, 5% purity) under N₂. The suspension was degassed under vacuum and purged with H₂ several times. The mixture was stirred under H₂ (50 psi) at 25° C. for 2 hours. TLC showed the reaction was completed. The mixture was filtered and concentrated in vacuum to give BzL-23d (0.3 g, 834.85 μmol, 81.26% yield) as a gray solid. ¹H NMR (DMSO-d₆, 400 MHz) δ 9.39-9.46 (m, 1H), 7.97 (t, J=5.2 Hz, 1H), 7.59 (d, J=2.8 Hz, 1H), 6.90 (dd, J=8.8, 2.8 Hz, 1H), 6.64 (d, J=8.8 Hz, 1H), 3.99 (d, J=12.8 Hz, 2H), 3.15-3.26 (m, 6H), 2.54-2.63 (m, 2H), 2.16-2.26 (m, 1H), 1.65-1.71 (m, 2H), 1.48-1.60 (m, 2H)

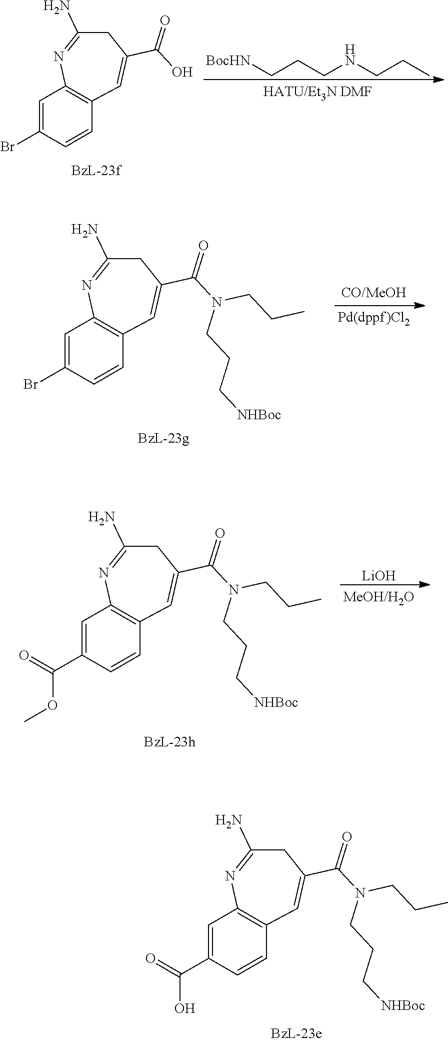

### Synthesis of tert-butyl (3-(2-amino-8-bromo-N-propyl-3H-benzo[b]azepine-4-carboxamido)propyl)carbamate, BzL-23g

To a mixture of 2-amino-8-bromo-3H-1-benzazepine-4-carboxylic acid, BzL-23f (4.09 g, 14.56 mmol, 1 eq) and tert-butyl N-[3-(propylamino)propyl]carbamate (3.78 g, 17.47 mmol, 1.2 eq) in DMF (10 mL) was added HATU (6.64 g, 17.47 mmol, 1.2 eq) and Et₃N (2.95 g, 29.12 mmol, 4.05 mL, 2 eq) in one portion at 25 C. The mixture was stirred at 25° C. for 1 h. LCMS showed the reaction was finished. The mixture was diluted with water and extracted with EtOAc (50 mL×3). The organic layer was washed with brine, dried over Na₂SO₄, filtered and concentrated. The residue was purified by silica gel chromatography (column height: 250 mm, diameter: 100 mm, 100-200 mesh silica gel, Petroleum ether/Ethyl acetate=1/0, 0/1) to afford BzL-23g (6 g, 12.52 mmol, 85.95% yield) as a yellow oil.

### Synthesis of methyl 2-amino-4-[3-(tert-butoxycarbonylamino)propyl-propyl-carbamoyl]-3H-1-benzazepine-8-carboxylate, BzL-23h

To a solution of tert-butyl N-[3-[(2-amino-8-bromo-3H-1-benzazepine-4-carbonyl)-propyl-amino]propyl]carbamate, BzL-23g (5 g, 10.43 mmol, 1 eq) in MeOH (50 mL) was added Et₃N (3.17 g, 31.29 mmol, 4.35 mL, 3 eq) and Pd(dppf)C₁₂ (763.13 mg, 1.04 mmol, 0.1 eq) under N₂. The suspension was degassed under vacuum and purged with CO (10.43 mmol, 1 eq) several times. The mixture was stirred under CO (50 psi) at 80° C. for 12 hours. LCMS showed the reaction was finished. The mixture was filtered and concentrated to give BzL-23h (7 g, crude) as yellow oil.

### Synthesis of 2-amino-4-((3-((tert-butoxycarbonyl)amino)propyl)(propyl)carbamoyl)-3H-benzo[b]azepine-8-carboxylic acid, BzL-23e

To a mixture of methyl 2-amino-4-[3-(tert-butoxycarbonylamino)propyl-propyl-carbamoyl]-3H-1-benzazepine-8-carboxylate, BzL-23h (6 g, 13.08 mmol, 1 eq) in MeOH (80 mL) was added LiOH (1.25 g, 52.34 mmol, 4 eq) in one portion at 30° C. The mixture was stirred at 30° C. for 12 h. LCMS showed the reaction was finished. The mixture was adjusted pH 6 with aq HCl (1 M) at 25° C. The mixture was concentrated. The mixture was further purification by pre-HPLC(column: Phenomenex luna C18 250×50 mm, 10 um (micron); mobile phase: [water(0.1% TFA)-ACN]; B %: 10%-40%, 20 min) to give BzL-23e (1.4 g, 3.09 mmol, 23.64% yield, 98.23% purity) as yellow oil. ¹H NMR (MeOD, 400 MHz) δ 8.06 (d, J=1.2 Hz, 1H), 8.02 (dd, J=1.6, 8.0 Hz, 1H), 7.68 (s, 1H), 7.14 (s, 1H), 3.58-3.44 (m, 4H), 3.37 (s, 2H), 3.10 (m, 2H), 1.85 (m, 2H), 1.71 (m, 2H), 1.51-1.33 (m, 9H), 0.92-0.98 (m, 3H). LC/MS [M+H]445.25 (calculated); LC/MS [M+H]445.10 (observed).

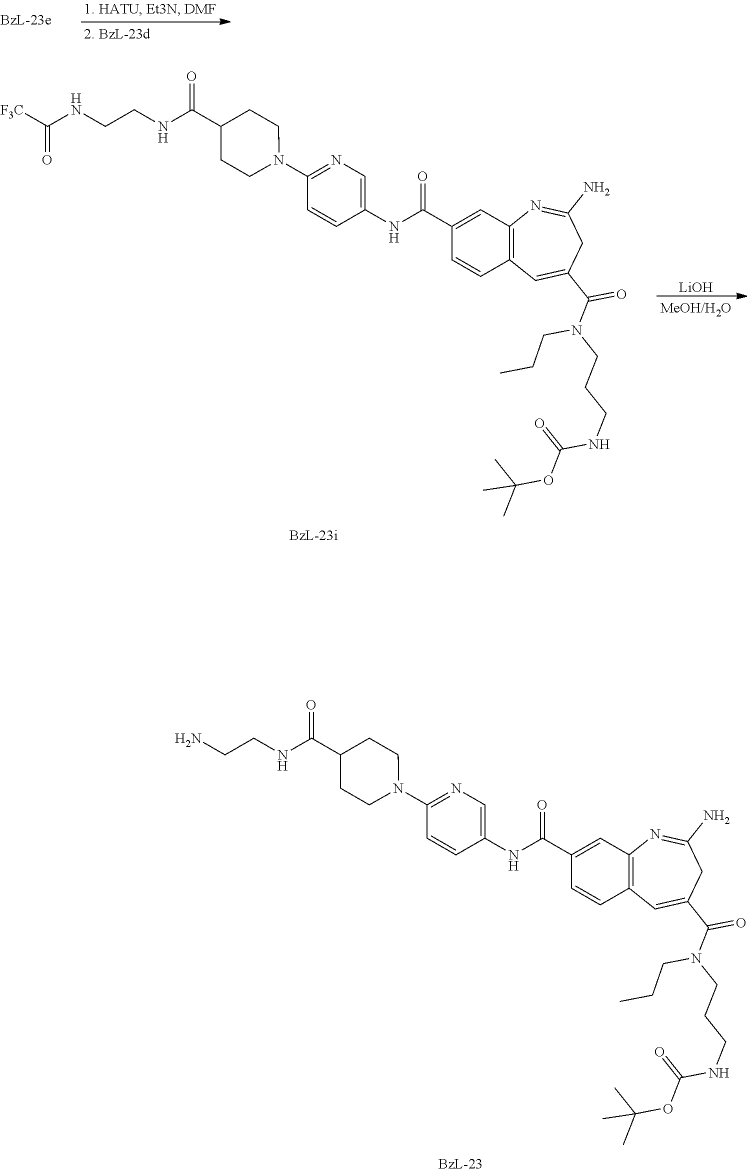

### Synthesis of tert-butyl (3-(2-amino-N-propyl-8-((6-(4-((2-(2,2,2-trifluoroacetamido)ethyl)carbamoyl)piperidin-1-yl)pyridin-3-yl)carbamoyl)-3H-benzo[b]azepine-4-carboxamido)propyl)carbamate, BzL-23i

To a mixture of 2-amino-4-[3-(tert-butoxycarbonylamino)propyl-propyl-carbamoyl]-3H-1-benzazepine-8-carboxylic acid, BzL-23e (200 mg, 449.92 μmol, 1 eq) HATU (205.29 mg, 539.90 μmol, 1.2 eq) in DMF (3 mL) was added Et₃N (136.58 mg, 1.35 mmol, 187.87 μL, 3 eq) at 25° C. The mixture was stirred at 25° C. for 5 min, then 1-(5-amino-2-pyridyl)-N-[2-[(2,2,2-trifluoroacetyl)amino]ethyl]piperidine-4-carboxamide, BzL-23d (161.68 mg, 449.92 μmol, 1 eq) was added to the mixture, stirred for 30 min. LCMS showed major as desired. The mixture was poured into water (50 mL). The aqueous phase was extracted with ethyl acetate (50 mL). The combined organic phase was washed with brine (50 mL), dried with anhydrous Na₂SO₄, filtered and concentrated in vacuum to give BzL-23i (0.3 g, 381.75 μmol, 84.85% yield) as yellow oil.

### Synthesis of tert-butyl (3-(2-amino-8-((6-(4-((2-aminoethyl)carbamoyl)piperidin-1-yl)pyridin-3-yl)carbamoyl)-N-propyl-3H-benzo[b]azepine-4-carboxamido)propyl)carbamate, BzL-23

To a mixture of tert-butyl N-[3-[[2-amino-8-[[6-[4-[2-[(2,2,2-trifluoroacetyl) amino]ethylcarbamoyl]-1-piperidyl]-3-pyridyl]carbamoyl]-3H-1-benzazepine-4-carbonyl]-propyl-amino]propyl]carbamate, BzL-23i (0.25 g, 318.13 μmol, 1 eq) in MeOH (10 mL) was added LiOH·H₂O (40.05 mg, 954.38 μmol, 3 eq) in H₂O (1 mL) at 25° C. The mixture was stirred at 40° C. for 12 hours. LCMS showed major as desired. The mixture was concentrated in vacuum. The residue was purified by prep-HPLC column: Nano-micro Kromasil C18 100×30 mm Sum; mobile phase: [water(0.1% TFA)-ACN]; B %: 15%-45%, 10 min to give BzL-23 (45 mg, 65.23 μmol, 20.51% yield) as a white solid. ¹H NMR (MeOD, 400 MHz) δ 8.73 (d, J=2.4 Hz, 1H), 8.24 (dd, J=9.8, 2.4 Hz, 1H), 7.75 (br s, 1H), 7.45 (d, J=9.8 Hz, 1H), 7.15 (br s, 1H), 4.24 (br d, J=13.6 Hz, 2H), 3.35-3.62 (m, 9H), 3.05-3.12 (m, 4H), 2.59-2.72 (m, 1H), 1.99-2.09 (m, 2H), 1.65-1.94 (m, 6H), 1.45 (s, 9H), 0.90-0.98 (m, 3H). LC/MS [M+H]690.41 (calculated); LC/MS [M+H]690.40 (observed).

### Example 47 Synthesis of BzL-24

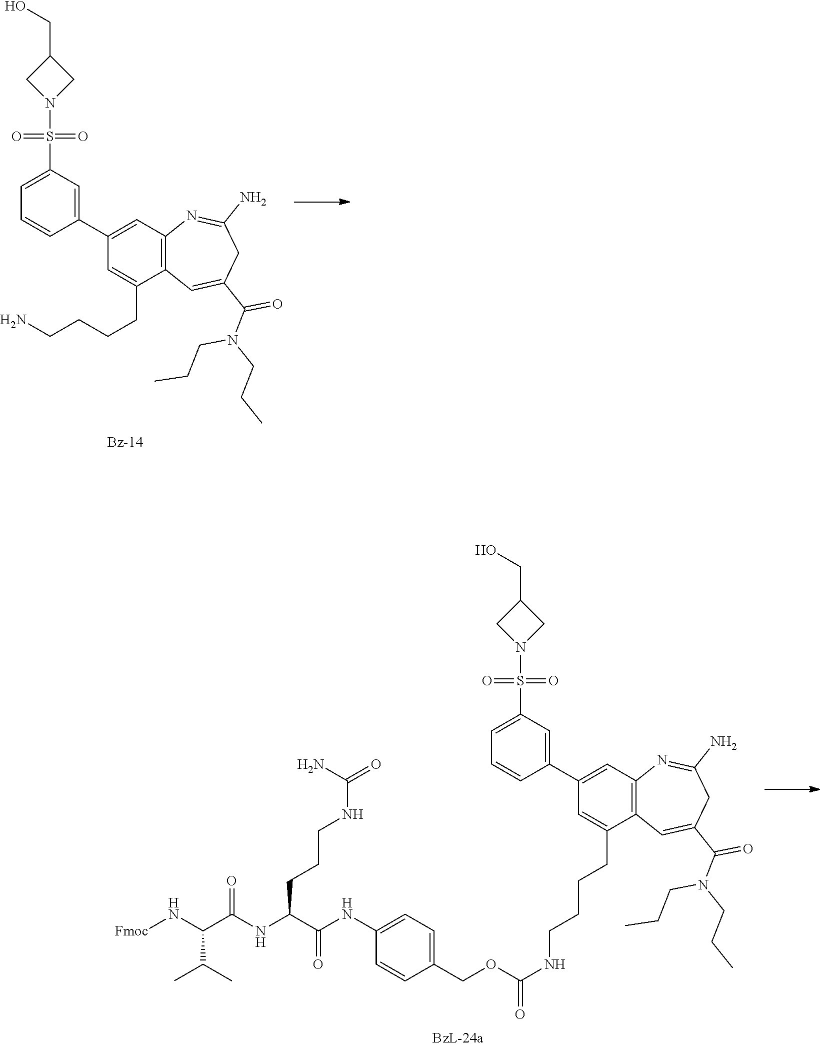

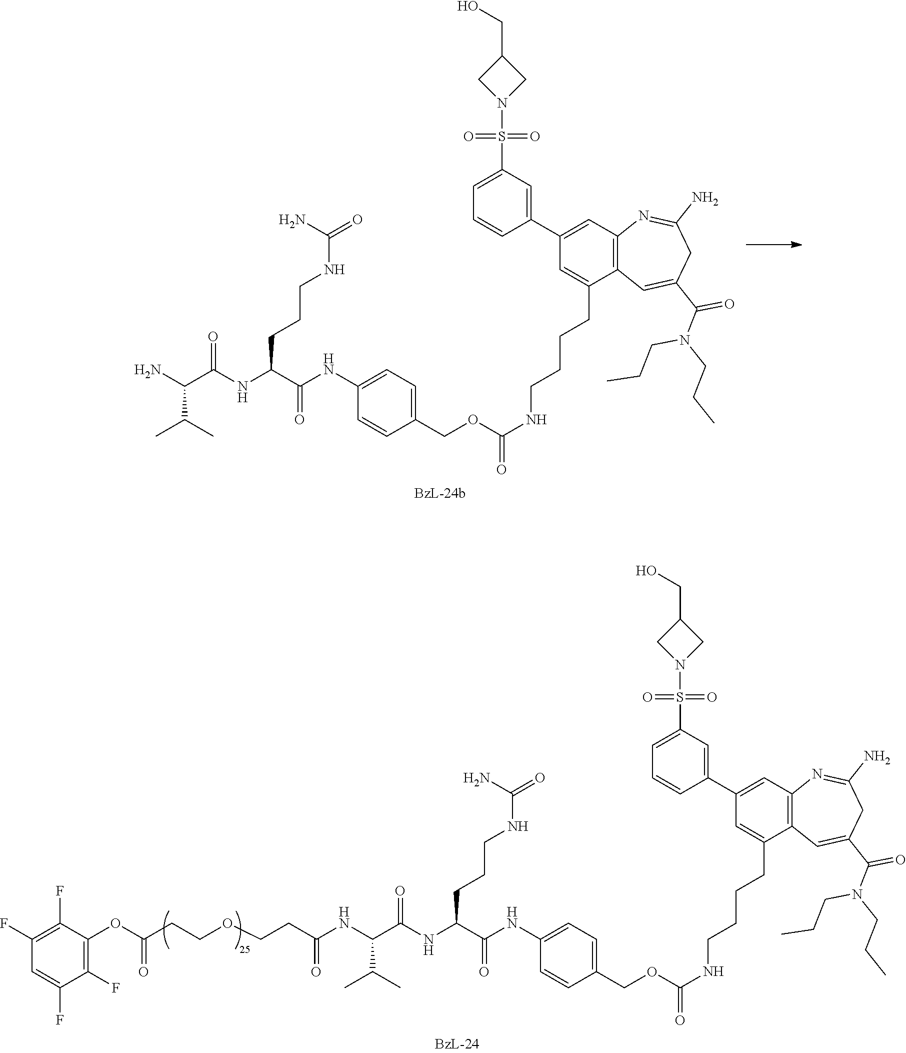

4-((S)-2-((S)-2-((((9H-Fluoren-9-yl)methoxy)carbonyl)amino)-3-methylbutanamido)-5-ureidopentanamido)benzyl (4-(2-amino-4-(dipropylcarbamoyl)-8-(3-((3-(hydroxymethyl)azetidin-1-yl)sulfonyl)phenyl)-3H-benzo[b]azepin-6-yl)butyl)carbamate, BzL-24a was synthesized from Bz-14 according to the procedure described for BzL-26a. LC/MS [M+H]1209.58 (calculated); LC/MS [M+H]1209.85 (observed).

4-((S)-2-((S)-2-Amino-3-methylbutanamido)-5-ureidopentanamido)benzyl (4-(2-amino-4-(dipropylcarbamoyl)-8-(3-((3-(hydroxymethyl)azetidin-1-yl)sulfonyl)phenyl)-3H-benzo[b]azepin-6-yl)butyl)carbamate, BzL-24b was synthesized according to the procedure described for BzL-26. LC/MS [M+H]987.51 (calculated); LC/MS [M+H]987.75 (observed).

2,3,5,6-Tetrafluorophenyl (6S,9S)-1-amino-6-((4-((((4-(2-amino-4-(dipropylcarbamoyl)-8-(3-((3-(hydroxymethyl)azetidin-1-yl)sulfonyl)phenyl)-3H-benzo[b]azepin-6-yl)butyl)carbamoyl)oxy)methyl)phenyl)carbamoyl)-9-isopropyl-1,8,11-trioxo-14,17,20,23,26,29,32,35,38,41,44,47,50,53,56,59,62,65,68,71,74,77,80,83,86-pentacosaoxa-2,7,10-triazanonaoctacontan-89-oate, BzL-24 was synthesized according to the procedure described for BzL-15. LC/MS [M+2H/2]1168.59 (calculated); LC/MS [M+2H/2]1169.36 (observed).

### Example 48 Synthesis of BzL-26

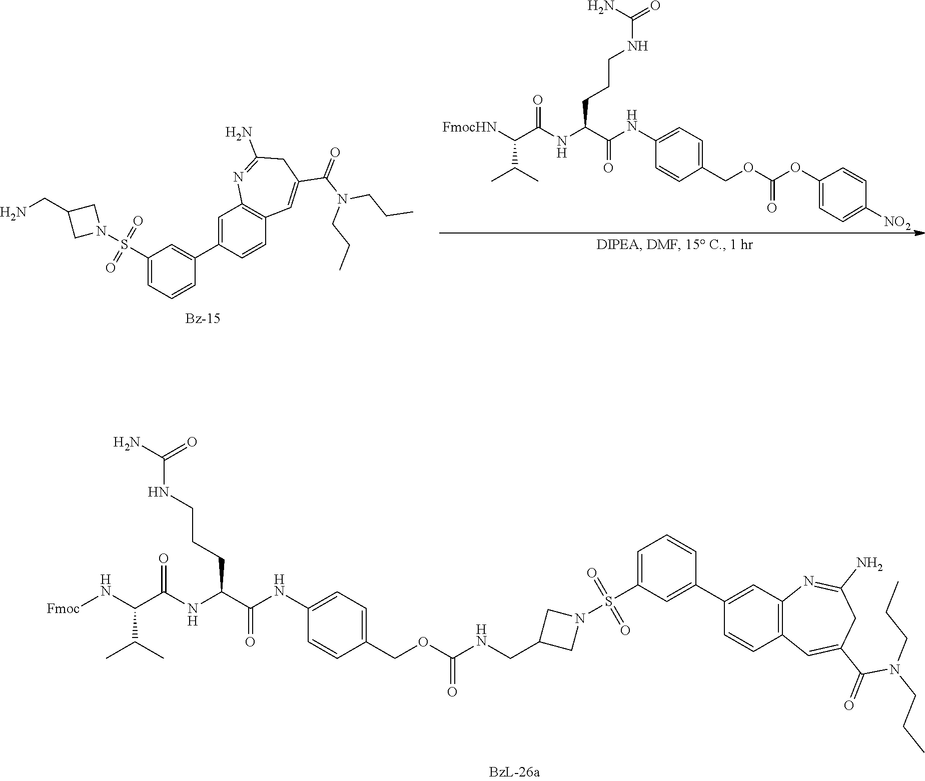

### Synthesis of (9H-fluoren-9-yl)methyl ((S)-1-(((S)-1-((4-(((((1-((3-(2-amino-4-(dipropylcarbamoyl)-3H-benzo[b]azepin-8-yl)phenyl)sulfonyl)azetidin-3-yl)methyl)carbamoyl)oxy)methyl)phenyl)amino)-1-oxo-5-ureidopentan-2-yl)amino)-3-methyl-1-oxobutan-2-yl)carbamate, BzL-26a

To a solution of [4-[[(2S)-2-[[(2S)-2-(9H-fluoren-9-ylmethoxycarbonylamino)-3-methyl-butanoyl]amino]-5-ureido-pentanoyl]amino]phenyl]methyl (4-nitrophenyl) carbonate (200 mg, 260.83 μmol, 1 eq) in DMF(1 mL) was added a solution of 2-amino-8-[3-[3-(aminomethyl)azetidin-1-yl]sulfonylphenyl]-N,N-dipropyl-3H-1-benzazepine-4-carboxamide, Bz-15 (325.35 mg, 521.65 μmol, 2 eq, TFA) and DIPEA (67.42 mg, 521.65 μmol, 90.86 μL, 2 eq) in DMF(1 mL) at 15° C. under N₂. The mixture was stirred at 15° C. for 1 hr. The mixture was filtered. The residue was purified by prep-HPLC (column: Nano-micro Kromasil C18 100×30 mm Sum; liquid phase: [A-TFA/H₂O=0.1% v/v; B-ACN]B %: 30%-60%, 12 min]) to give [4-[[(2S)-2-[[(2S)-2-(9H-fluoren-9-ylmethoxycarbonylamino)-3-methyl-butanoyl]amino]-5-ureido-pentanoyl]amino]phenyl]methylN-[[1-[3-[2-amino-4-(dipropylcarbamoyl)-3H-1-benzazepin-8-yl]phenyl]sulfonylazetidin-3-yl]methyl]carbamate, BzL-26a (73 mg, 63.07 μmol, 24.18% yield, 98.259% purity) as white solid. ¹H NMR (MeOD-d₄, 400 MHz) δ 8.05-8.09 (m, 1H), 7.92-7.98 (m, 1H), 7.84-7.90 (m, 1H), 7.58-7.83 (m, 8H), 7.46-7.57 (m, 2H), 7.33-7.42 (m, 2H), 7.25-7.33 (m, 2H), 7.11-7.23 (m, 2H), 7.04-7.09 (m, 1H), 4.87-4.94 (m, 2H), 4.46-4.56 (m, 1H), 4.31-4.45 (m, 2H), 4.16-4.26 (m, 1H), 3.95 (br d, J=7.0 Hz, 1H), 3.85 (br t, J=8.0 Hz, 2H), 3.52-3.63 (m, 2H), 3.46 (br d, J=2.0 Hz, 4H), 3.35 (s, 3H), 3.15-3.23 (m, 1H), 3.01-3.13 (m, 3H), 2.58-2.71 (m, 1H), 2.00-2.16 (m, 1H), 1.84-1.96 (m, 1H), 1.64-1.77 (m, 4H), 1.49-1.62 (m, 2H), 0.75-1.09 (m, 12H) LC/MS [M+H]1137.52 (calculated); LC/MS [M+H]1137.10 (observed).

### Synthesis of 4-((S)-2-((S)-2-amino-3-methylbutanamido)-5-ureidopentanamido)benzyl ((1-((3-(2-amino-4-(dipropylcarbamoyl)-3H-benzo[b]azepin-8-yl)phenyl)sulfonyl)azetidin-3-yl)methyl)carbamate, BzL-26

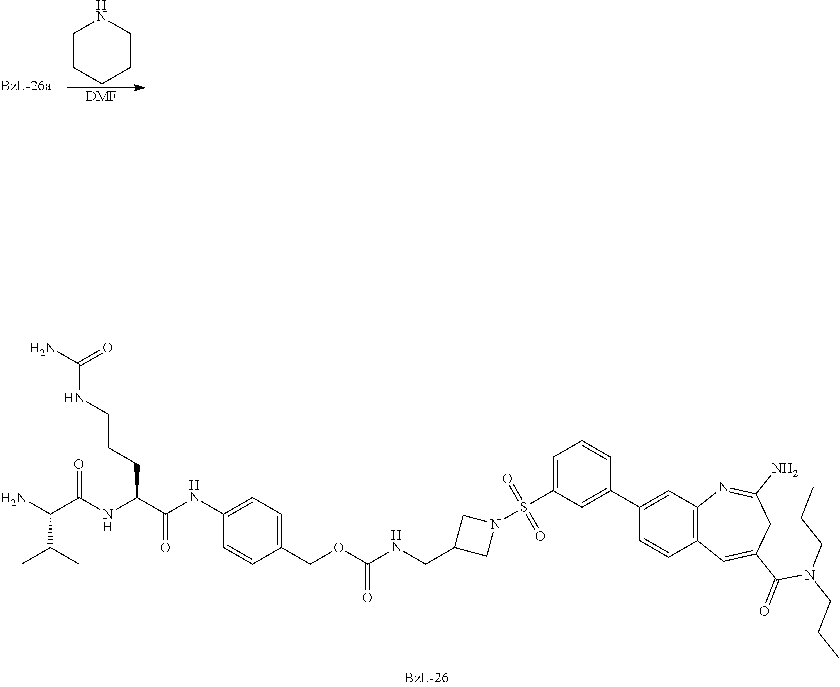

To a solution of [4-[[(2S)-2-[[(2S)-2-(9H-fluoren-9-ylmethoxycarbonylamino)-3-methyl-butanoyl]amino]-5-ureido-pentanoyl]amino]phenyl]methylN-[[1-[3-[2-amino-4-(dipropylcarbamoyl)-3H-1-benzazepin-8-yl]phenyl]sulfonylazetidin-3-yl]methyl]carbamate, BzL-26a (0.12 g, 105.51 μmol, 1 eq) in DMF (2 mL) was added piperidine (44.92 mg, 527.54 μmol, 52.10 μL, 5 eq) at 25° C. and stirred for 1 hour. The reaction mixture was filtered and the filter was concentrated. The residue was purified by prep-HPLC (column: Welch Xtimate C18 100×25 mm×3 um; mobile phase: [water (10 mM NH₄HCO₃)-ACN]; B %: 25%-65%, 12 min). Compound [4-[[(2S)-2-[[(2S)-2-amino-3-methyl-butanoyl]amino]-5-ureido-pentanoyl]amino]phenyl]methylN-[[1-[3-[2-amino-4-(dipropylcarbamoyl)-3H-1-benzazepin-8-yl]phenyl]sulfonylazetidin-3-yl]methyl]carbamate, BzL-26 (0.037 g, 38.51 μmol, 36.50% yield, 95.25% purity) was obtained as a yellow solid. ¹H NMR (MeOD, 400 MHz) δ 8.06 (s, 1H), 7.98 (d, J=7.4 Hz, 1H), 7.82 (d, J=7.4 Hz, 1H), 7.74 (t, J=7.4 Hz, 1H), 7.54 (d, J=8.4 Hz, 2H), 7.50-7.43 (m, 2H), 7.38 (d, J=8.0 Hz, 1H), 7.23 (d, J=8.8 Hz, 2H), 6.90 (s, 1H), 4.95-4.90 (m, 2H), 4.62-4.54 (m, 2H), 3.84 (t, J=8.2 Hz, 2H), 3.56 (t, J=4.2 Hz, 2H), 3.44 (t, J=4.0 Hz, 4H), 3.23 (d, J=5.2 Hz, 2H), 3.14-3.03 (m, 2H), 2.68-2.62 (m, 1H), 2.04-1.99 (m, 2H), 1.92-1.84 (m, 2H), 1.79-1.47 (m, 8H), 1.08-0.75 (m, 12H). LC/MS [M+H]915.46 (calculated); LC/MS [M+H]915.10 (observed).

### Example 49 Synthesis of BzL-27

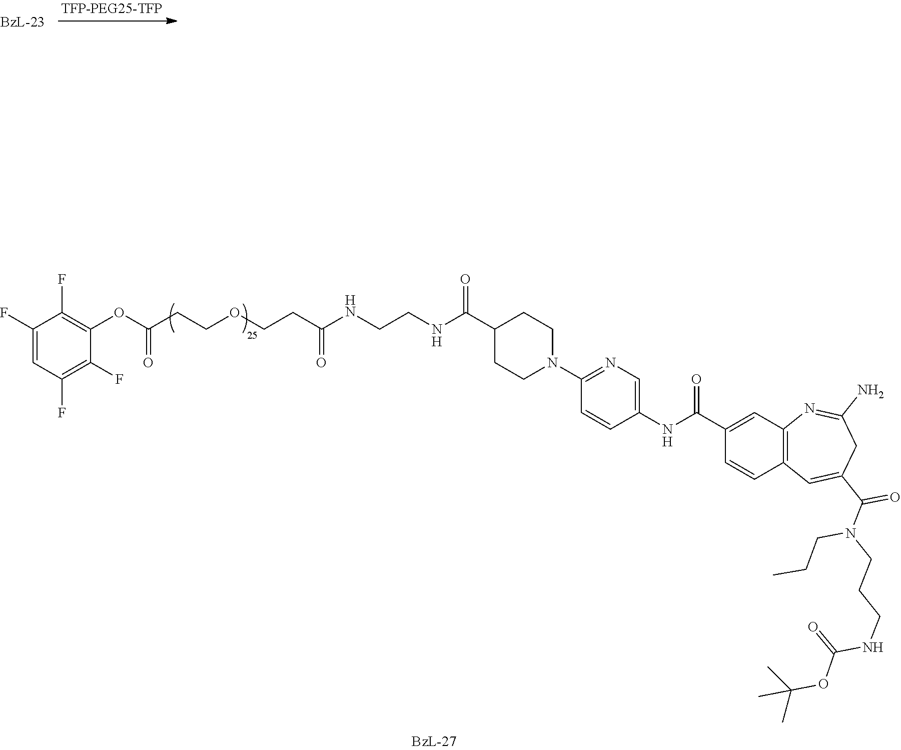

2,3,5,6-Tetrafluorophenyl 1-(1-(5-(2-amino-4-((3-((tert-butoxycarbonyl)amino)propyl)(propyl)carbamoyl)-3H-benzo[b]azepine-8-carboxamido)pyridin-2-yl)piperidin-4-yl)-1,6-dioxo-9,12,15,18,21,24,27,30,33,36,39,42,45,48,51,54,57,60,63,66,69,72,75,78,81-pentacosaoxa-2,5-diazatetraoctacontan-84-oate, BzL-27 was synthesized from BzL-23 and TFP-PEG25-TFP according to the procedure described for Bz-31. LC/MS [M+H]2039.07 (calculated); LC/MS [M+H]2039.40 (observed).

### Example 50 Synthesis of BzL-28

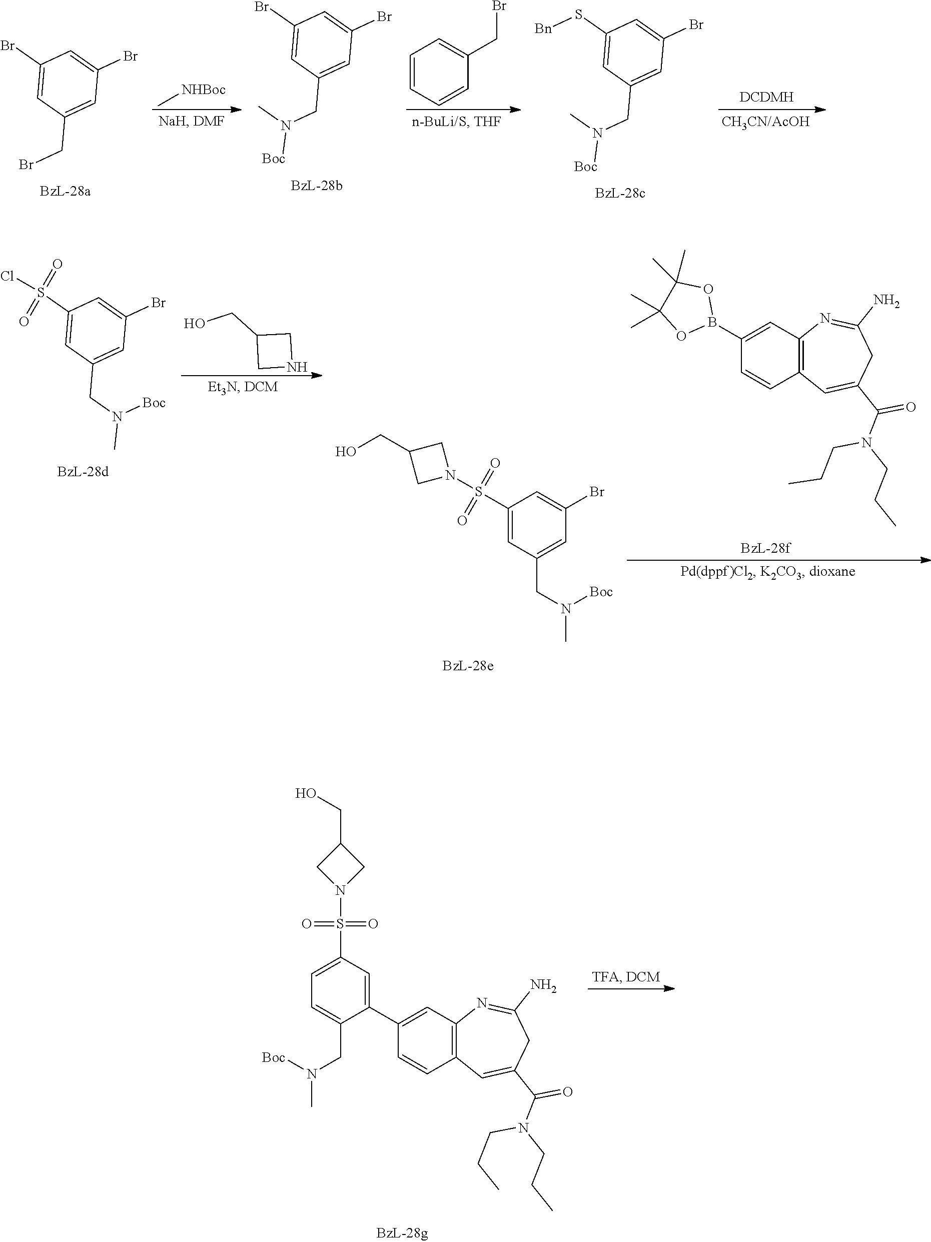

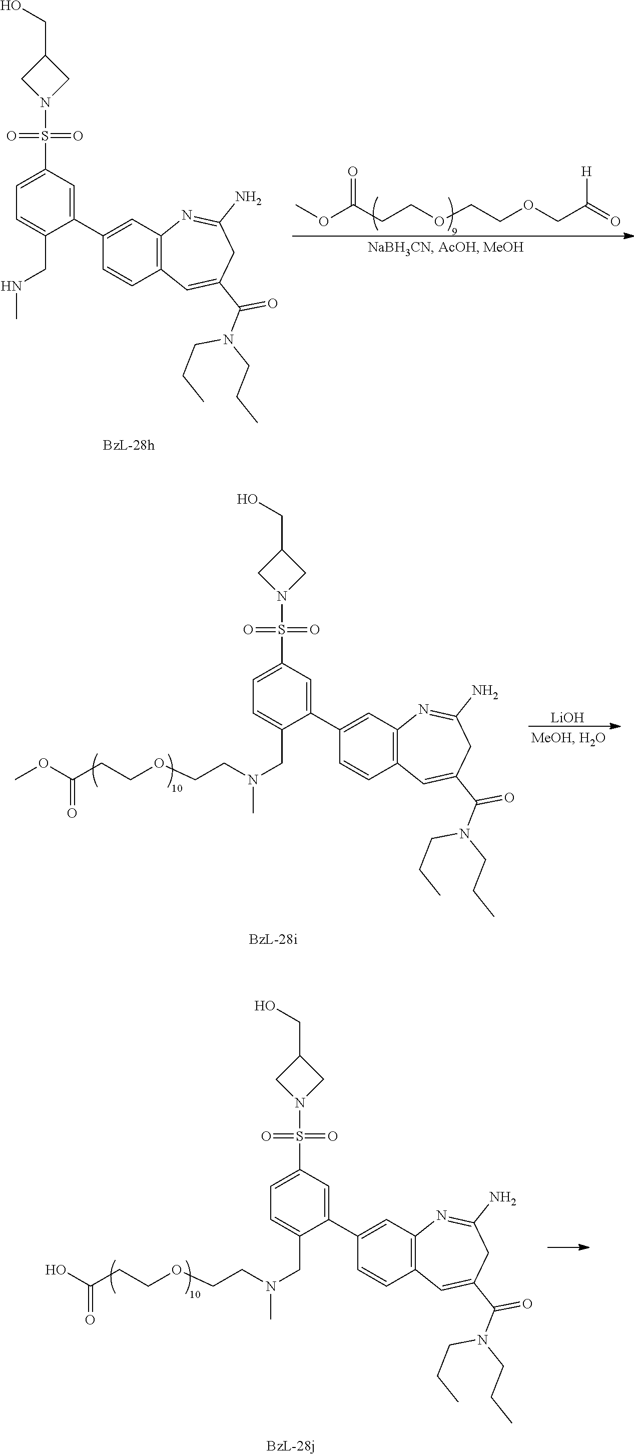

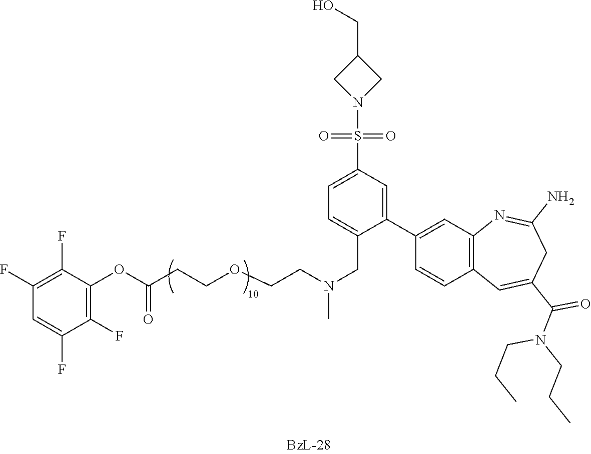

### Synthesis of tert-butyl 3,5-dibromobenzyl(methyl)carbamate, BzL-28b

To a solution of tert-butyl N-methylcarbamate (2.5 g, 19.06 mmol, 1 eq) in DMF (80 mL) was added NaH (914.82 mg, 22.87 mmol, 60% purity, 1.2 eq) slowly at 0° C. Afte addition, the mixture was stirred at 15° C. for 30 min, and then 1,3-dibromo-5-(bromomethyl)benzene, BzL-28a (8.77 g, 26.68 mmol, 1.4 eq) was added at 0° C. The resulting mixture was stirred at 15° C. for 2 h. TLC indicated the reactant was consumed completely. The reaction mixture was quenched by addition of aq. NH₄Cl (250 mL) at 0° C., and then extracted with EtOAc (100 mL×3). The combined organic layers were washed with brine (30 mL×3) dried over Na₂SO₄, filtered and concentrated under reduced pressure to give a residue. The residue was purified by column chromatography (SiO₂, Petroleum ether:Ethyl acetate=1:0 to 5:1) to give BzL-28b (6.6 g, 17.41 mmol, 91.35% yield) as a white solid. ¹H NMR (CDCl₃, 400 MHz) δ 7.59-7.56 (m, 1H), 7.31 (s, 2H), 4.36 (s, 2H), 2.87 (s, 3H), 1.49 (s, 9H).

### Synthesis of tert-butyl 3-(benzylthio)-5-bromobenzyl(methyl)carbamate, BzL-28c

To a solution of tert-butyl 3,5-dibromobenzyl(methyl)carbamate, BzL-28b (3.6 g, 9.50 mmol, 1 eq) in THE (70 mL) was added dropwise n-BuLi (2.5 M, 3.80 mL, 1 eq) at −78° C. under N₂. After addition, the mixture was stirred at −78° C. for 15 min, and then sulfur, S (304.55 mg, 9.50 mmol, 1 eq) was added at −78° C. After addition, the mixture was stirred at −78° C. for 45 min, and then bromomethylbenzene (1.62 g, 9.50 mmol, 1.13 mL, 1 eq) was added at −78° C. The resulting mixture was warmed to 15° C. and stirred at 15° C. for 30 min. TLC indicated BzL-28b was consumed completely. The reaction mixture was quenched by addition of aq. NH₄Cl (70 mL) at 0° C., and then extracted with EtOAc (50 mL×3). The combined organic layers were washed with brine (20 mL), dried over Na₂SO₄, filtered and concentrated under reduced pressure to give a residue. The residue was purified by column chromatography (SiO₂, Petroleum ether:Ethyl acetate=1:0 to 5:1) to give BzL-28c (0.97 g, 2.30 mmol, 24.18% yield) as a yellow oil. ¹H NMR (CDCl₃, 400 MHz) δ 7.35-7.26 (m, 5H), 7.26-7.21 (m, 1H), 7.17 (s, 1H), 7.04 (s, 1H), 4.34 (s, 2H), 4.12 (s, 2H), 2.79 (s, 3H), 1.48 (s, 9H).

### Synthesis of tert-butyl 3-bromo-5-(chlorosulfonyl)benzyl(methyl)carbamate, BzL-28d

To a solution of tert-butyl 3-(benzylthio)-5-bromobenzyl(methyl)carbamate, BzL-28c (1.22 g, 2.89 mmol, 1 eq) in CH₃CN (25 mL) and H₂O (1 mL) and acetic acid, AcOH (520.35 mg, 8.67 mmol, 495.57 μL, 3 eq) was added 1,3-dichloro-5,5-dimethyl-imidazolidine-2,4-dione, DCDMH (1.14 g, 5.78 mmol, 2 eq) at 0° C. The mixture was stirred at 0° C. for 1 h. TLC indicated BzL-28c was consumed completely. The reaction mixture was concentrated under reduced pressure to give a residue. The residue was diluted with H₂O (20 mL) and extracted with EtOAc (20 mL×3). The combined organic layers were washed with brine (10 mL), dried over Na₂SO₄, filtered and concentrated under reduced pressure to give a residue. The residue was purified by column chromatography (SiO₂, Petroleum ether:Ethyl acetate=1:0 to 5:1) to give BzL-28d (0.51 g, 1.28 mmol, 44.29% yield) as a light yellow oil. ¹H NMR (CDCl₃, 400 MHz) δ 8.08 (s, 1H), 7.83 (s, 1H), 7.74 (s, 1H), 4.50 (s, 2H), 2.91 (s, 3H), 1.49 (s, 9H).

### Synthesis of tert-butyl 3-bromo-5-((3-(hydroxymethyl)azetidin-1-yl)sulfonyl)benzyl(methyl)carbamate, BzL-28e

To a solution of tert-butyl 3-bromo-5-(chlorosulfonyl)benzyl(methyl)carbamate, BzL-28d (0.74 g, 1.86 mmol, 1 eq) and azetidin-3-ylmethanol (746.66 mg, 3.71 mmol, 2 eq, TFA) in DCM (15 mL) was added TEA (751.25 mg, 7.42 mmol, 1.03 mL, 4 eq) at 0° C. The mixture was stirred at 15° C. for 1 h. TLC indicated Reactant 1 was consumed completely. The reaction mixture was quenched by addition of H₂O (15 mL) at 0° C., and then extracted with EtOAc (15 mL×3). The combined organic layers were washed with brine (10 mL), dried over Na₂SO₄, filtered and concentrated under reduced pressure to give a residue purified by column chromatography (SiO₂, Petroleum ether:Ethyl acetate=10:1 to 0:1) to give BzL-28e (640 mg, 1.42 mmol, 76.74% yield) as a light yellow oil. ¹H NMR (CDCl₃, 400 MHz) δ 7.90 (s, 1H), 7.69-7.53 (m, 2H), 4.48 (s, 2H), 3.89 (t, J=8.0 Hz, 2H), 3.64 (d, J=6.0 Hz, 3H), 3.42 (s, 1H), 2.95 (s, 3H), 2.65 (s, 1H), 1.49 (s, 9H).

### Synthesis of tert-butyl 3-(2-amino-4-(dipropylcarbamoyl)-3H-benzo[b]azepin-8-yl)-5-((3-(hydroxymethyl)azetidin-1-yl)sulfonyl)benzyl(methyl)carbamate, BzL-28g

A mixture of tert-butyl 3-bromo-5-((3-(hydroxymethyl)azetidin-1-yl)sulfonyl)benzyl(methyl)carbamate, BzL-28e (590 mg, 1.31 mmol, 1 eq), 2-amino-N,N-dipropyl-8-(4,4,5,5-tetramethyl-1,3,2-dioxaborolan-2-yl)-3H-benzo[b]azepine-4-carboxamide, BzL-28f (702.11 mg, 1.71 mmol, 1.3 eq), Pd(dppf)C₁₂ (48.0 mg, 65.7 μmol, 0.05 eq), K₂CO₃ (362.9 mg, 2.63 mmol, 2 eq) in dioxane (10 mL) and H₂O (1 mL) was degassed and purged with N₂ for 3 times, and then the mixture was stirred at 90° C. for 3 h under N₂ atmosphere. The reaction mixture was filtered and concentrated under reduced pressure to give a residue. The residue was purified by prep-HPLC (TFA condition: column: Nano-micro Kromasil C18 100×30 mm, Sum; mobile phase: [water(0.1% TFA)-ACN]; B %: 40%-60%, 10 min) to give BzL-28g (180 mg, 275.30 μmol, 20.97% yield) as a yellow solid.

### Synthesis of 2-amino-8-(3-((3-(hydroxymethyl)azetidin-1-yl)sulfonyl)-5-((methylamino)methyl)phenyl)-N,N-dipropyl-3H-benzo[b]azepine-4-carboxamide, BzL-28h

To a solution of tert-butyl 3-(2-amino-4-(dipropylcarbamoyl)-3H-benzo[b]azepin-8-yl)-5-((3-(hydroxymethyl)azetidin-1-yl)sulfonyl)benzyl(methyl)carbamate, BzL-28g (180 mg, 275.30 μmol, 1 eq) in DCM (2 mL) was added TFA (627.80 mg, 5.51 mmol, 407.66 μL, 20 eq) at 15° C. The mixture was stirred at 15° C. for 1 h. LC-MS showed Reactant 1 was consumed. The reaction mixture was concentrated under reduced pressure to give a residue. The residue was added with THE (5 mL) and aq. NaHCO₃ (5 mL) to pH 8-9 at 0° C., and then stirred at 15° C. for 30 min. The reaction mixture was concentrated under reduced pressure to give a residue and extracted with EtOAc (10 mL×3). The combined organic layers were washed with brine (5 mL), dried over Na₂SO₄, filtered and concentrated under reduced pressure to give BzL-28h (110 mg, 198.66 μmol, 72.16% yield) as a yellow oil. LC/MS [M+H]554.28 (calculated); LC/MS [M+H]554.30 (observed).

### Synthesis of methyl 1-(3-(2-amino-4-(dipropylcarbamoyl)-3H-benzo[b]azepin-8-yl)-5-((3-(hydroxymethyl)azetidin-1-yl)sulfonyl)phenyl)-2-methyl-5,8,11,14,17,20,23,26,29,32-decaoxa-2-azapentatriacontan-35-oate, BzL-28i

To a solution of 2-amino-8-(3-((3-(hydroxymethyl)azetidin-1-yl)sulfonyl)-5-((methylamino)methyl)phenyl)-N,N-dipropyl-3H-benzo[b]azepine-4-carboxamide, BzL-28h (110 mg, 198.66 μmol, 1 eq) and methyl 1-oxo-3,6,9,12,15,18,21,24,27,30-decaoxatritriacontan-33-oate (140.13 mg, 258.26 μmol, 1.3 eq) in MeOH (2 mL) was added AcOH (11.93 mg, 198.6 μmol, 11.36 μL, 1 eq) at 15° C. After addition, the mixture was stirred at 15° C. for 15 min, and then NaBH₃CN (24.97 mg, 397.32 μmol, 2 eq) was added at 15° C. The resulting mixture was stirred at 15° C. for 12 h. The reaction mixture was used for next step directly, containing BzL-28i (0.22 g, crude) (in MeOH) as a light yellow liquid. LC/MS [M+2H/2]540.79 (calculated); LC/MS [M+H]541.1 (observed).

### Synthesis of 1-(3-(2-amino-4-(dipropylcarbamoyl)-3H-benzo[b]azepin-8-yl)-5-((3-(hydroxymethyl)azetidin-1-yl)sulfonyl)phenyl)-2-methyl-5,8,11,14,17,20,23,26,29,32-decaoxa-2-azapentatriacontan-35-oic acid, BzL-28j

To a solution of methyl 1-(3-(2-amino-4-(dipropylcarbamoyl)-3H-benzo[b]azepin-8-yl)-5-((3-(hydroxymethyl)azetidin-1-yl)sulfonyl)phenyl)-2-methyl-5,8,11,14,17,20,23,26,29,32-decaoxa-2-azapentatriacontan-35-oate, BzL-28i (0.22 g, 203.64 μmol, 1 eq) in MeOH (2 mL) and H₂O (1 mL) was added LiOH·H₂O (68.36 mg, 1.63 mmol, 8 eq) at 15° C. The mixture was stirred at 15° C. for 5 h. LC-MS showed BzL-28i was consumed. The reaction mixture was adjusted to pH 6-7 with 1 N HCl at 0° C., and then concentrated under reduced pressure. The residue was purified by prep-HPLC (TFA condition: column: Welch Xtimate C18 100×25 mm, 3 um; mobile phase: [water(0.1% TFA)-ACN]; B %: 20%-40%, 12 min) twice to give BzL-28j (104 mg, 94.31 μmol, 46.31% yield, HCl) as a light yellow oil. ¹H NMR (MeOD-d₄, 400 MHz) δ 8.33 (s, 1H), 8.24 (s, 1H), 8.12 (s, 1H), 7.90-7.84 (m, 2H), 7.74 (d, J=8.8 Hz, 1H), 7.12 (s, 1H), 3.96-3.88 (m, 4H), 3.76-3.67 (m, 8H), 3.66-3.52 (m, 33H), 3.51-3.37 (m, 9H), 3.02 (s, 3H), 2.71-2.59 (m, 1H), 2.53 (t, J=6.0 Hz, 2H), 1.77-1.63 (m, 4H), 0.95 (br s, 6H). LC/MS [M+H]1066.56 (calculated); LC/MS [M+H]1066.10 (observed).

2,3,5,6-Tetrafluorophenyl 1-(3-(2-amino-4-(dipropylcarbamoyl)-3H-benzo[b]azepin-8-yl)-5-((3-(hydroxymethyl)azetidin-1-yl)sulfonyl)phenyl)-2-methyl-5,8,11,14,17,20,23,26,29,32-decaoxa-2-azapentatriacontan-35-oate, BzL-28 was synthesized by reaction with 2,3,5,6-tetrafluorophenol according to the procedure described for BzL-22. LC/MS [M+H]1214.56 (calculated); LC/MS [M+H]1214.83 (observed).

### Example 51 Synthesis of BzL-29

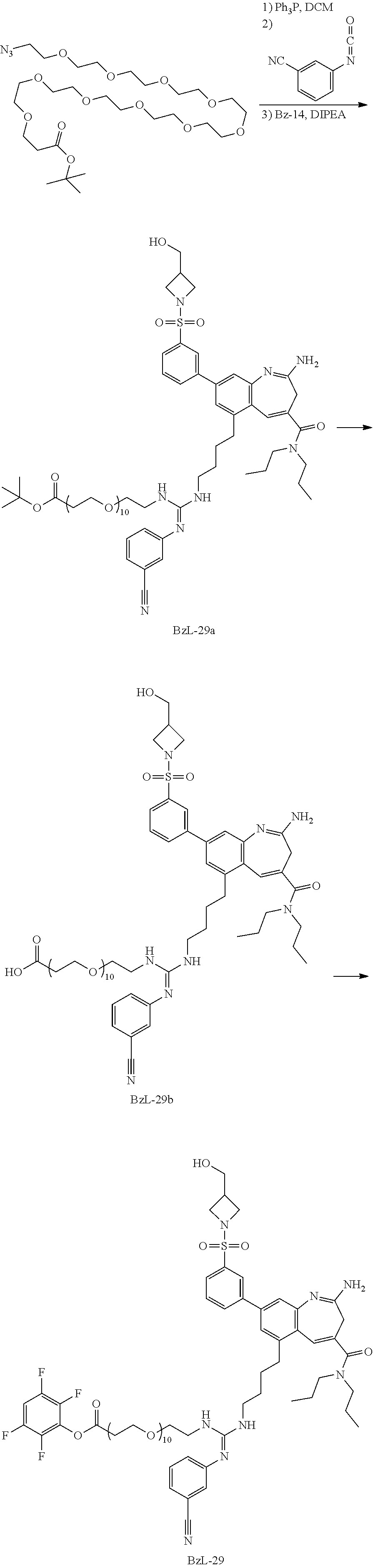

### Synthesis of tert-butyl (Z)-40-(2-amino-4-(dipropylcarbamoyl)-8-(3-((3-(hydroxymethyl)azetidin-1-yl)sulfonyl)phenyl)-3H-benzo[b]azepin-6-yl)-35-((3-cyanophenyl)imino)-4,7,10,13,16,19,22,25,28,31-decaoxa-34,36-diazatetracontanoate, BzL-29a

A 4 mL vial was charged with tert-butyl 1-azido-3,6,9,12,15,18,21,24,27,30-decaoxatritriacontan-33-oate (0.011 mmol, 6.9 mg), triphenylphosphine (0.011 mmol, 3 mg) and 200 μL of anhydrous dichloromethane. The reaction was maintained at 30° C. for 90 min, at which point 3-cyanophenyl isocyanate (0.011 mmol, 1.6 mg) was added. After 45 min a solution containing Bz-14 (0.011 mmol) and diisopropylethylamine, Hunigs base (0.034 mmol) in 200 μL DMF was added. This reaction was maintained for 2 h then concentrated under reduced pressure. The crude reaction was purified using reverse phase preparative HPLC utilizing a 25-75% gradient of acetonitrile:water containing 0.1% trifluoroacetic acid. The purified fractions were combined and lyophilized to afford 4.1 mg of BzL-29a in 63% yield. LC/MS [M+H]1293.71 (calculated); LC/MS [M+H]1294.04 (observed).

### Synthesis of (Z)-40-(2-amino-4-(dipropylcarbamoyl)-8-(3-((3-(hydroxymethyl)azetidin-1-yl)sulfonyl)phenyl)-3H-benzo[b]azepin-6-yl)-35-((3-cyanophenyl)imino)-4,7,10,13,16,19,22,25,28,31-decaoxa-34,36-diazatetracontanoic acid, BzL-29b

A vial was charged with BzL-29a (4.1 mg, 0.003 mmol), 500 μL DCM, and 100 μL trifluoroacetic acid. The reaction was maintained for 1 h, concentrated under reduced pressure, and azeotroped thrice with 1 mL toluene. The crude product BzL-29b was taken onto the subsequent step.

2,3,5,6-Tetrafluorophenyl (Z)-40-(2-amino-4-(dipropylcarbamoyl)-8-(3-((3-(hydroxymethyl)azetidin-1-yl)sulfonyl)phenyl)-3H-benzo[b]azepin-6-yl)-35-((3-cyanophenyl)imino)-4,7,10,13,16,19,22,25,28,31-decaoxa-34,36-diazatetracontanoate, BzL-29 was synthesized by reaction of BzL-29b with 2,3,5,6-tetrafluorophenol according to the procedure described for Bz-22. LC/MS [M+H]1385.64 (calculated); LC/MS [M+H]1385.84 (observed).

### Example 52 Synthesis of BzL-31

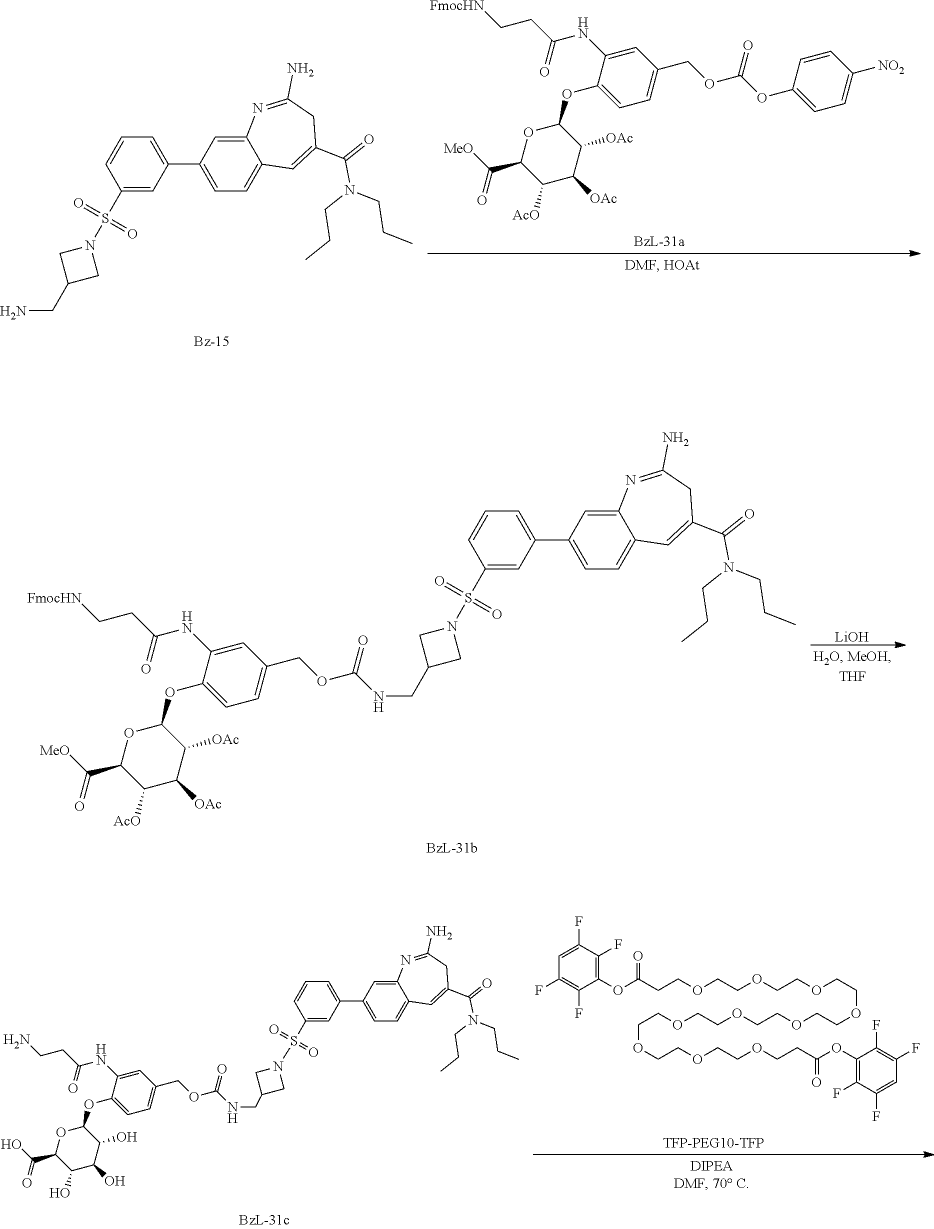

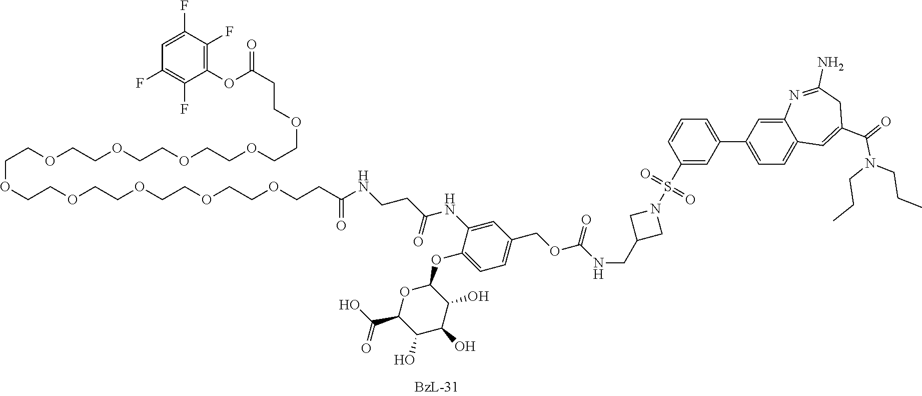 [SMILES: CCCN(CCC)C(=O)C1=Cc2ccc(-c3cccc(S(=O)(=O)N4CC(CNC(=O)OCc5ccc(O[C@@H]6O[C@H](C(=O)O)[C@@H](O)[C@H](O)[C@H]6O)c(NC(=O)CCNC(=O)CCOCCOCCOCCOCCOCCOCCOCCOCCOCCOCCC(=O)Oc6c(F)c(F)cc(F)c6F)c5)C4)c3)cc2N=C(N)C1]

### Synthesis of rac-(2R,3S,4R,5R,6R)-2-(2-(3-((((9H-fluoren-9-yl)methoxy)carbonyl)amino)propanamido)-4-(((((1-((3-(2-amino-4-(dipropylcarbamoyl)-3H-benzo[b]azepin-8-yl)phenyl)sulfonyl)azetidin-3-yl)methyl)carbamoyl)oxy)methyl)phenoxy)-6-(methoxycarbonyl)tetrahydro-2H-pyran-3,4,5-triyl triacetate, BzL-31b

To a solution of Bz-15 (50 mg, 0.098 mmol, 1 eq) and rac-(2R,3S,4R,5R,6R)-2-(2-(3-((((9H-fluoren-9-yl)methoxy)carbonyl)amino)propanamido)-4-((((4-nitrophenoxy)carbonyl)oxy)methyl)phenoxy)-6-(methoxycarbonyl)tetrahydro-2H-pyran-3,4,5-triyl triacetate, BzL-31a (90 mg, 0.098 mmol, 1 eq) in DMF (0.2 ml) was added HOAt (13.3 mg, 0.098 mmol, 1 eq). The reaction was stirred at ambient temperature and monitored by LCMS. The reaction mixture was diluted with 1:1 water:acetonitrile and purified by HPLC to give BzL-31b (67 mg, 0.052 mmol, 53%). LC/MS [M+H]1284.48 (calculated); LC/MS [M+H]1284.81 (observed).

### Synthesis of rac-(2R,3R,4R,5S,6R)-6-(4-(((((1-((3-(2-amino-4-(dipropylcarbamoyl)-3H-benzo[b]azepin-8-yl)phenyl)sulfonyl)azetidin-3-yl)methyl)carbamoyl)oxy)methyl)-2-(3-aminopropanamido)phenoxy)-3,4,5-trihydroxytetrahydro-2H-pyran-2-carboxylic acid, BzL-31c

BzL-31b (67 mg, 0.052 mmol, 1 eq) was dissolved in a 20 mM solution of LiOH in 5:2:1 THF:MeOH:H₂O (2.6 ml). The reaction was stirred for 1 hour at ambient temperature, then concentrated and purified by HPLC to give BzL-31c as a white solid (25 mg, 0.027 mmol, 52%). LC/MS [M+H]922.37 (calculated); LC/MS [M+H]922.56 (observed).

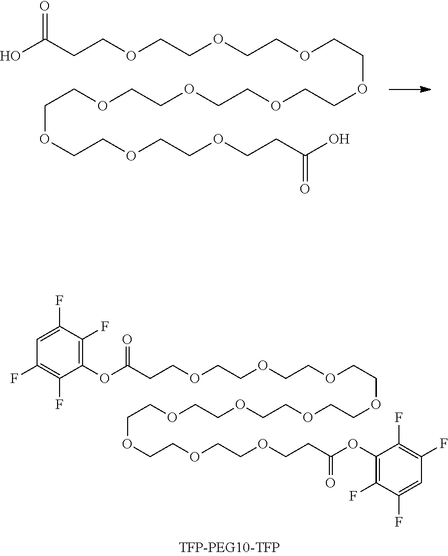 [SMILES: O=C(O)CCOCCOCCOCCOCCOCCOCCOCCOCCOCCOCCC(=O)O ; O=C(CCOCCOCCOCCOCCOCCOCCOCCOCCOCCOCCC(=O)Oc1c(F)c(F)cc(F)c1F)Oc1c(F)c(F)cc(F)c1F]

Bis(2,3,5,6-tetrafluorophenyl) 4,7,10,13,16,19,22,25,28,31-decaoxatetratriacontanedioate, TFP-PEG10-TFP was synthesized from 4,7,10,13,16,19,22,25,28,31-decaoxatetratriacontanedioic acid according to the procedure described for TFP-PEG25-TFP. LC/MS [M+H]855.28 (calculated); LC/MS [M+H]855.53 (observed).

### Synthesis of rac-(2R,3R,4R,5S,6R)-6-(4-(((((1-((3-(2-amino-4-(dipropylcarbamoyl)-3H-benzo[b]azepin-8-yl)phenyl)sulfonyl)azetidin-3-yl)methyl)carbamoyl)oxy)methyl)-2-(1,34-dioxo-1-(2,3,5,6-tetrafluorophenoxy)-4,7,10,13,16,19,22,25,28,31-decaoxa-35-azaoctatriacontan-38-amido)phenoxy)-3,4,5-trihydroxytetrahydro-2H-pyran-2-carboxylic acid, BzL-31

BzL-31c (25 mg, 0.027 mmol, 1 eq) and TFP-PEG10-TFP bis(2,3,5,6-tetrafluorophenyl) 4,7,10,13,16,19,22,25,28,31-decaoxatetratriacontanedioate (35 mg, 0.040 mmol, 1.5 eq) were dissolved in DMF (5 ml). The reaction was neutralized to approximately pH 7 with DIPEA and heated to 70° C. After 1 hour, another portion of bis(2,3,5,6-tetrafluorophenyl) 4,7,10,13,16,19,22,25,28,31-decaoxatetratriacontanedioate (35 mg, 0.040 mmol, 1.5 eq) was added to the reaction mixture. Upon consumption of BzL-31c, the reaction was concentrated to a yellow film, then triturated with 6×3 ml diethyl ether to give a yellow solid that was purified by HPLC to give BzL-31 (14.3 mg, 0.0089 mmol, 33%). LC/MS [M+H]1610.64 (calculated); LC/MS [M+H]1610.99 (observed).

### Example 53 Synthesis of BzL-33

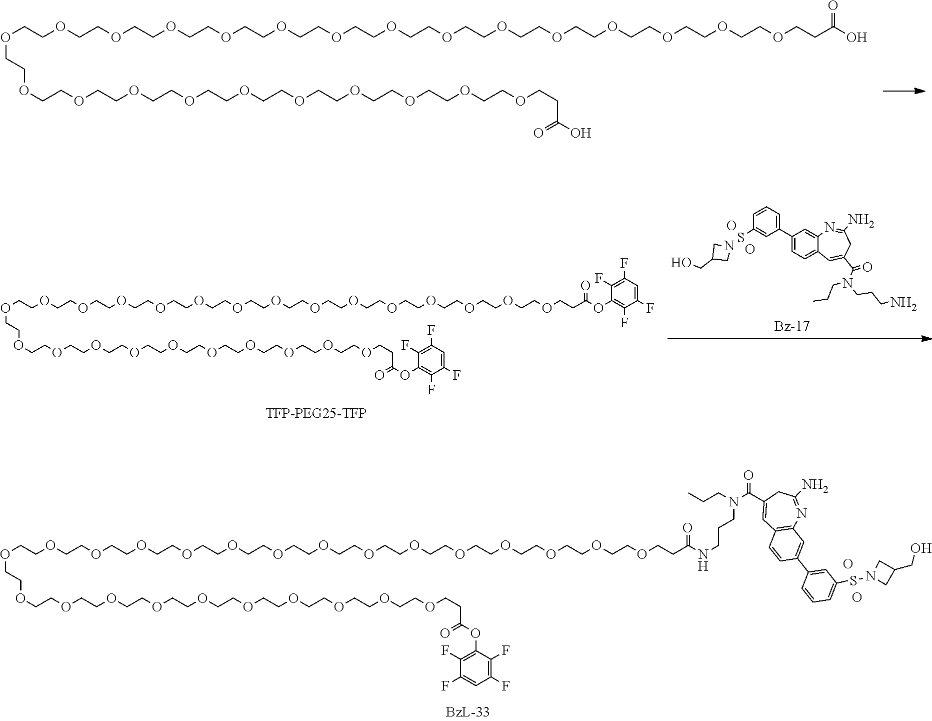

A vial was charged with 4,7,10,13,16,19,22,25,28,31,34,37,40,43,46,49,52,55,58,61,64,67,70,73,76-pentacosaoxanonaheptacontanedioic acid (269 mg, 0.221 mmol), 2,3,5,6-tetrafluorophenol (110 mg, 0.662 mmol), collidine (176 μL, 1.33 mmol), 1-ethyl-3-(3-dimethylaminopropyl)carbodiimide (127 mg, 0.221 mmol) and 3 mL DMF. The reaction was stirred for 16 h, then purified by reverse phase preparative HPLC utilizing a 25-75% gradient of acetonitrile:water containing 0.1% trifluoroacetic acid. The purified fractions were combined and lyophilized to afford 266 mg of bis(2,3,5,6-tetrafluorophenyl) 4,7,10,13,16,19,22,25,28,31,34,37,40,43,46,49,52,55,58,61,64,67,70,73,76-pentacosaoxanonaheptacontanedioate, TFP-PEG25-TFP in 79% yield. LC/MS [M+H]1515.68 (calculated); LC/MS [M+H]1516.00 (observed).

A vial was charged with 2-amino-N-(3-aminopropyl)-8-[3-[3-(hydroxymethyl)azetidin-1-yl]sulfonylphenyl]-N-propyl-3H-1-benzazepine-4-carboxamide, Bz-17 (0.0275 mmol), TFP-PEG25-TFP (0.0275 mmol), collidine (0.0825 mmol) in 300 μL DMF. The reaction was maintained for 5 h and then purified by reverse phase preparative HPLC utilizing a 25-75% gradient of acetonitrile:water containing 0.1% trifluoroacetic acid. The purified fractions were combined and lyophilized to afford 8.2 mg of 2,3,5,6-tetrafluorophenyl 84-(2-amino-8-(3-((3-(hydroxymethyl)azetidin-1-yl)sulfonyl)phenyl)-3H-benzo[b]azepine-4-carbonyl)-79-oxo-4,7,10,13,16,19,22,25,28,31,34,37,40,43,46,49,52,55,58,61,64,67,70,73,76-pentacosaoxa-80,84-diazaheptaoctacontanoate, BzL-33 in 25% yield. LC/MS [M+H]1874.9 (calculated); LC/MS [M+H]1874.9 (observed).

### Example 54 Synthesis of BzL-34

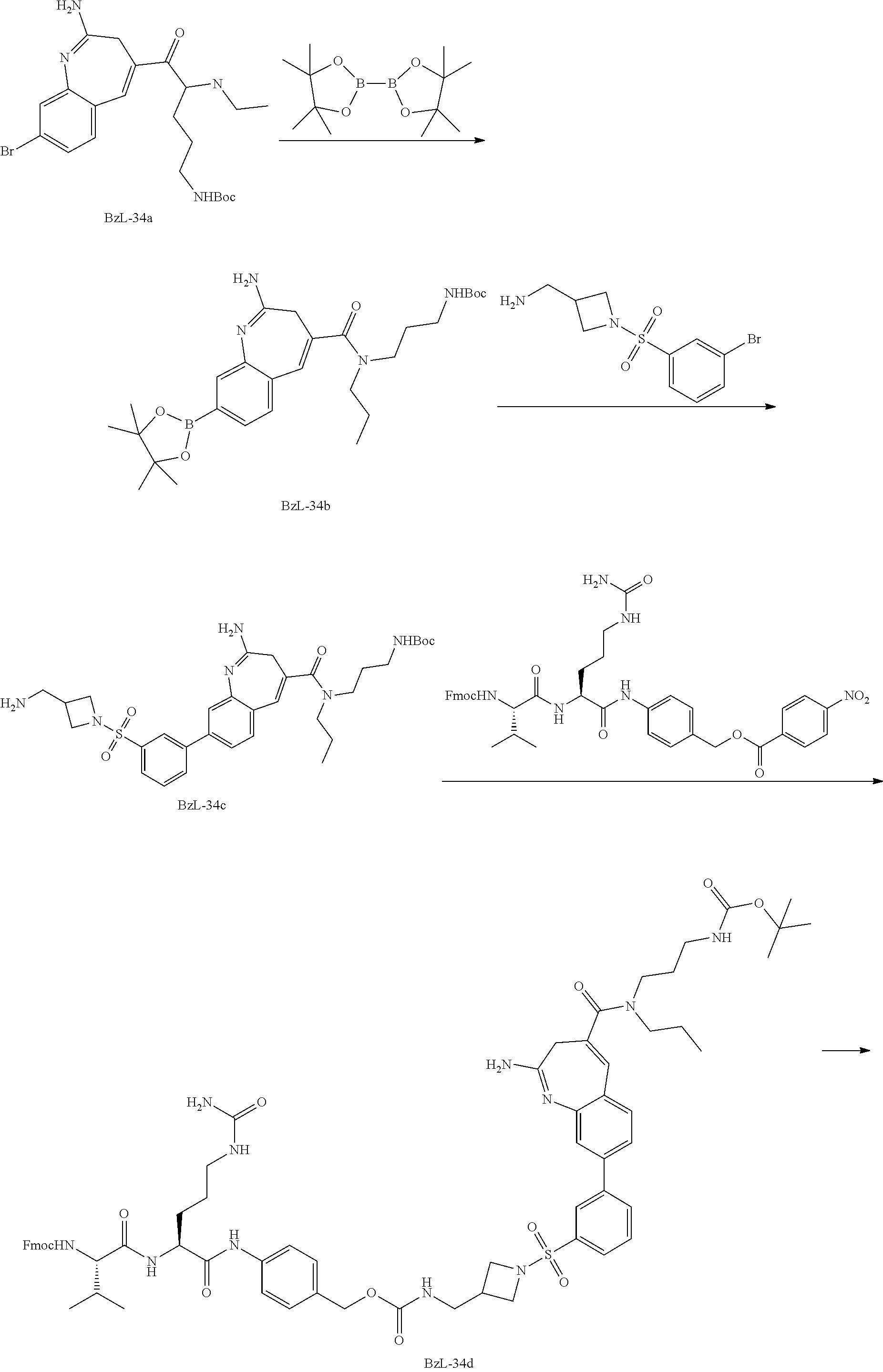

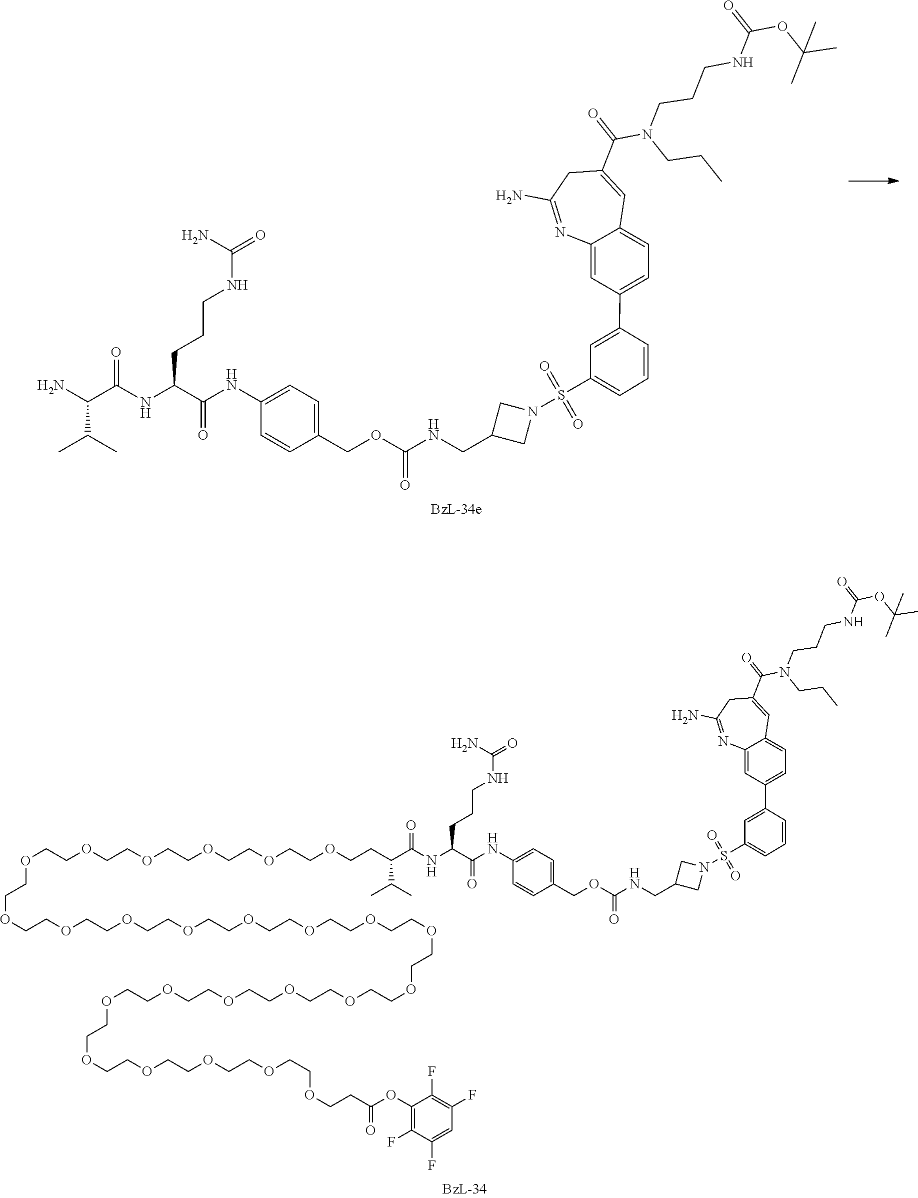 [SMILES: CCCN(CCCNC(=O)OC(C)(C)C)C(=O)C1=Cc2ccc(-c3cccc(S(=O)(=O)N4CC(CNC(=O)OCc5ccc(NC(=O)[C@H](CCCNC(N)=O)NC(=O)[C@@H](N)C(C)C)cc5)C4)c3)cc2N=C(N)C1 ; CCCN(CCCNC(=O)OC(C)(C)C)C(=O)C1=Cc2ccc(-c3cccc(S(=O)(=O)N4CC(CNC(=O)OCc5ccc(NC(=O)[C@H](CCCNC(N)=O)NC(=O)[C@@H](CCOCCOCCOCCOCCOCCOCCOCCOCCOCCOCCOCCOCCOCCOCCOCCOCCOCCOCCOCCOCCOCCOCCOCCOCCOCCC(=O)Oc6c(F)c(F)cc(F)c6F)C(C)C)cc5)C4)c3)cc2N=C(N)C1]

Preparation of BzL-34b: To a mixture of tert-butyl N-[3-[(2-amino-8-bromo-3H-1-benzazepine-4-carbonyl)-propyl-amino]propyl]carbamate, BzL-34a (0.80 g, 1.67 mmol, 1.0 eq) in dioxane (10 mL) was added 4,4,4′,4′,5,5,5′,5′-octamethyl-2,2′-bi(1,3,2-dioxaborolane), Pin₂B2 (509 mg, 2.00 mmol, 1.2 eq), KOAc (246 mg, 2.50 mmol, 1.5 eq) and Pd(dppf)C₁₂ (122 mg, 167 umol, 0.1 eq) in one portion at 15° C. under N₂ and then stirred at 90° C. for 12 h. The mixture was filtered and concentrated to give tert-butyl N-[3-[[2-amino-8-(4,4,5,5-tetramethyl-1,3,2-dioxaborolan-2-yl)-3H-1-benzazepine-4-carbonyl]-propyl-amino]propyl]carbamate, BzL-34b(0.90 g, crude) as black solid.

Preparation of BzL-34c: To a mixture of [1-(3-bromophenyl)sulfonylazetidin-3-yl]methanamine (0.40 g, 1.17 mmol, 1 eq, HCl) and BzL-34b (493 mg, 937 umol, 0.8 eq) in dioxane (4 mL) was added a solution of K₂CO₃ (728 mg, 5.27 mmol, 4.5 eq) in H₂O (0.4 mL) and Pd(dppf)C₁₂ (85.7 mg, 117 umol, 0.1 eq) at 15° C. under N₂ and then stirred at 90° C. for 2 h. The mixture was filtered and concentrated. The residue was purified by prep-HPLC(column: Welch Xtimate C18 100*25 mm*3 um; mobile phase: [water(0.1% TFA)-ACN]; B %: 20%-45%, 10.5 min) to give tert-butyl N-[3-[[2-amino-8-[3-[3-(aminomethyl)azetidin-1-yl]sulfonylphenyl]-3H-1-benzazepine-4-carbonyl]-propyl-amino]propyl]carbamate, BzL-34c (0.223 g, 357 umol, 30.5% yield) as white solid. ¹H NMR (MeOD, 400 MHz) δ8.14-8.07 (m, 2H), 7.92 (d, J=8.0 Hz, 1H), 7.86-7.81 (m, 1H), 7.79-7.70 (m, 3H), 7.12 (s, 1H), 3.96 (t, J=8.4 Hz, 2H), 3.65 (dd, J=5.2, 8.4 Hz, 2H), 3.58-3.42 (m, 4H), 3.37 (s, 2H), 3.06 (d, J=7.2 Hz, 4H), 1.90-1.78 (m, 2H), 1.74-1.64 (m, 2H), 1.44 (s, 9H), 0.96-0.90 (m, 3H). LC/MS [M+H]625.3 (calculated); LC/MS [M+H]625.0 (observed).

Preparation of BzL-34d: To a mixture of BzL-34c (0.18 g, 288 umol, 1.0 eq) and [4-[[(2S)-2-[[(2S)-2-(9H-fluoren-9-ylmethoxycarbonylamino)-3-methyl-butanoyl]amino]-5-ureido-pentanoyl]amino]phenyl]methyl (4-nitrophenyl) carbonate (176.7 mg, 230 umol, 0.8 eq) in DMF (2 mL) was added DIEA (74.5 mg, 576 umol, 100 μL, 2.0 eq) in one portion at 15° C. The mixture was stirred at the same temperature for 0.5 h. Then it was filtered and purified by prep-HPLC (column: Welch Xtimate C18 150*25 mm*5 um; mobile phase: [water (10 mM NH₄HCO₃)-ACN]; B %: 55%-75%, 10.5 min) to give [4-[[(2S)-2-[[(2S)-2-(9H-fluoren-9-ylmethoxycarbonylamino)-3-methyl-butanoyl]amino]-5-ureido-pentanoyl]amino]phenyl]methyl N-[[1-[3-[2-amino-4-[3-(tert-butoxycarbonylamino)propyl-propyl-carbamoyl]-3H-1-benzazepin-8-yl]phenyl]sulfonylazetidin-3-yl]methyl]carbamate, BzL-34d (0.024 g, 19.16 umol, 6.65% yield) as yellow solid. ¹H NMR (MeOH, 400 MHz) δ8.04 (s, 1H), 7.95 (d, J=6.4 Hz, 1H), 7.81-7.79 (m, 3H), 7.73 (d, J=7.6 Hz, 1H), 7.65 (t, J=6.8 Hz, 2H), 7.54 (d, J=8.0 Hz, 2H), 7.48-7.43 (m, 2H), 7.41-7.33 (m, 3H), 7.32-7.27 (m, 2H), 7.20 (d, J=8.0 Hz, 2H), 6.91 (s, 1H), 4.59 (s, 2H), 4.52 (s, 1H), 4.42-4.32 (m, 2H), 4.24-4.17 (m, 1H), 3.95 (d, J=7.2 Hz, 1H), 3.86-3.77 (m, 2H), 3.58-3.47 (m, 4H), 3.46-3.39 (m, 2H), 3.19-3.02 (m, 6H), 2.62 (d, J=7.6 Hz, 1H), 2.13-2.01 (m, 1H), 1.97-1.80 (m, 3H), 1.66 (s, 3H), 1.57 (s, 2H), 1.49-1.28 (m, 8H), 1.00-0.95 (m, 10H). LC/MS [M+H]1252.6 (calculated); LC/MS [M+H]1252.2 (observed).

Preparation of BzL-34e: A vial was charged with Bz-34d (20 mg, 0.016 mmol), diethylamine (0.08 mmol) and 150 μL DMF. The reaction was maintained for 6 h, then concentrated under reduced pressure to give 4-((S)-2-((S)-2-amino-3-methylbutanamido)-5-ureidopentanamido)benzyl ((1-((3-(2-amino-4-((3-((tert-butoxycarbonyl)amino)propyl)(propyl)carbamoyl)-3H-benzo[b]azepin-8-yl)phenyl)sulfonyl)azetidin-3-yl)methyl)carbamate, BzL-34e which was used in the subsequent step without further purification.

Preparation of BzL-34: Using the procedures described for BzL-33,2,3,5,6-tetrafluorophenyl (6S,9S)-1-amino-6-((4-(((((1-((3-(2-amino-4-((3-((tert-butoxycarbonyl)amino)propyl)(propyl)carbamoyl)-3H-benzo[b]azepin-8-yl)phenyl)sulfonyl)azetidin-3-yl)methyl)carbamoyl)oxy)methyl)phenyl)carbamoyl)-9-isopropyl-1,8,11-trioxo-14,17,20,23,26,29,32,35,38,41,44,47,50,53,56,59,62,65,68,71,74,77,80,83,86-pentacosaoxa-2,7,10-triazanonaoctacontan-89-oate, BzL-34 was obtained. LC/MS [M+H]2379.2 (calculated); LC/MS [M+2H/2]1190.1 (observed).

### Example 55 Synthesis of BzL-35

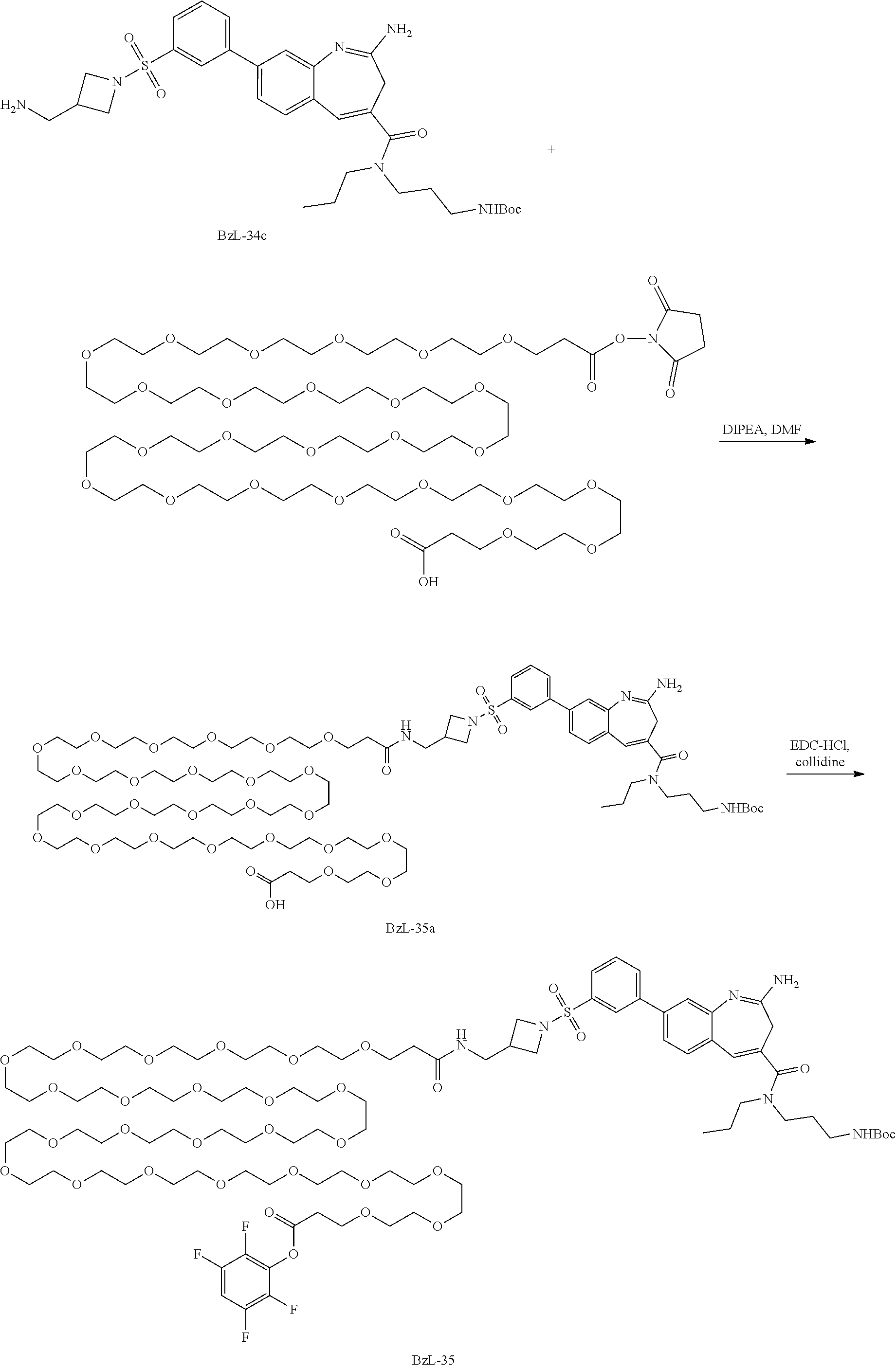

Preparation of BzL-35a: tert-Butyl (3-(2-amino-8-(3-((3-(aminomethyl)azetidin-1-yl)sulfonyl)phenyl)-N-propyl-3H-benzo[b]azepine-4-carboxamido)propyl)carbamate, BzL-34c (0.04 g, 0.064 mmol, 1 eq.) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (0.084 mg, 0.064 mmol, 1 eq.) were dissolved in DMF with diisopropylethylamine (0.033 ml, 0.192 mmol, 3 eq.). The reaction was monitored by LCMS and purified by HPLC to give 1-(1-((3-(2-amino-4-((3-((tert-butoxycarbonyl)amino)propyl)(propyl)carbamoyl)-3H-benzo[b]azepin-8-yl)phenyl)sulfonyl)azetidin-3-yl)-3-oxo-6,9,12,15,18,21,24,27,30,33,36,39,42,45,48,51,54,57,60,63,66,69,72,75,78-pentacosaoxa-2-azahenoctacontan-81-oic acid, BzL-35a (0.056, 0.031 mmol, 48%). LC/MS [M+H]1825.99 (calculated); LC/MS [M+H]1826.24 (observed).

Preparation of BzL-35: BzL-35a (0.060 g, 0.033 mmol, 1 eq.) and 2,3,5,6-tetrafluorophenol, TFP (0.011 g, 0.065 mmol, 2 eq.) were dissolved in 1 ml DMF. Collidine, 2,4,6-trimethylpyridine (0.022 ml, 0.16 mmol, 5 eq.) was added, followed by N-(3-dimethylaminopropyl)-N-ethylcarbodiimide hydrochloride, EDC-HCl, CAS Reg. No. 25952-53-8 (0.019 g, 0.098 mmol, 3 eq.). The reaction was stirred at room temperature and monitored by LCMS, then concentrated and purified by HPLC to give 2,3,5,6-tetrafluorophenyl 1-(1-((3-(2-amino-4-((3-((tert-butoxycarbonyl)amino)propyl)(propyl)carbamoyl)-3H-benzo[b]azepin-8-yl)phenyl)sulfonyl)azetidin-3-yl)-3-oxo-6,9,12,15,18,21,24,27,30,33,36,39,42,45,48,51,54,57,60,63,66,69,72,75,78-pentacosaoxa-2-azahenoctacontan-81-oate, BzL-35 (0.027 g, 0.014 mmol, 42%). LC/MS [M+H]1973.98 (calculated); LC/MS [M+H]1974.62 (observed).

### Example 56 Synthesis of BzL-36

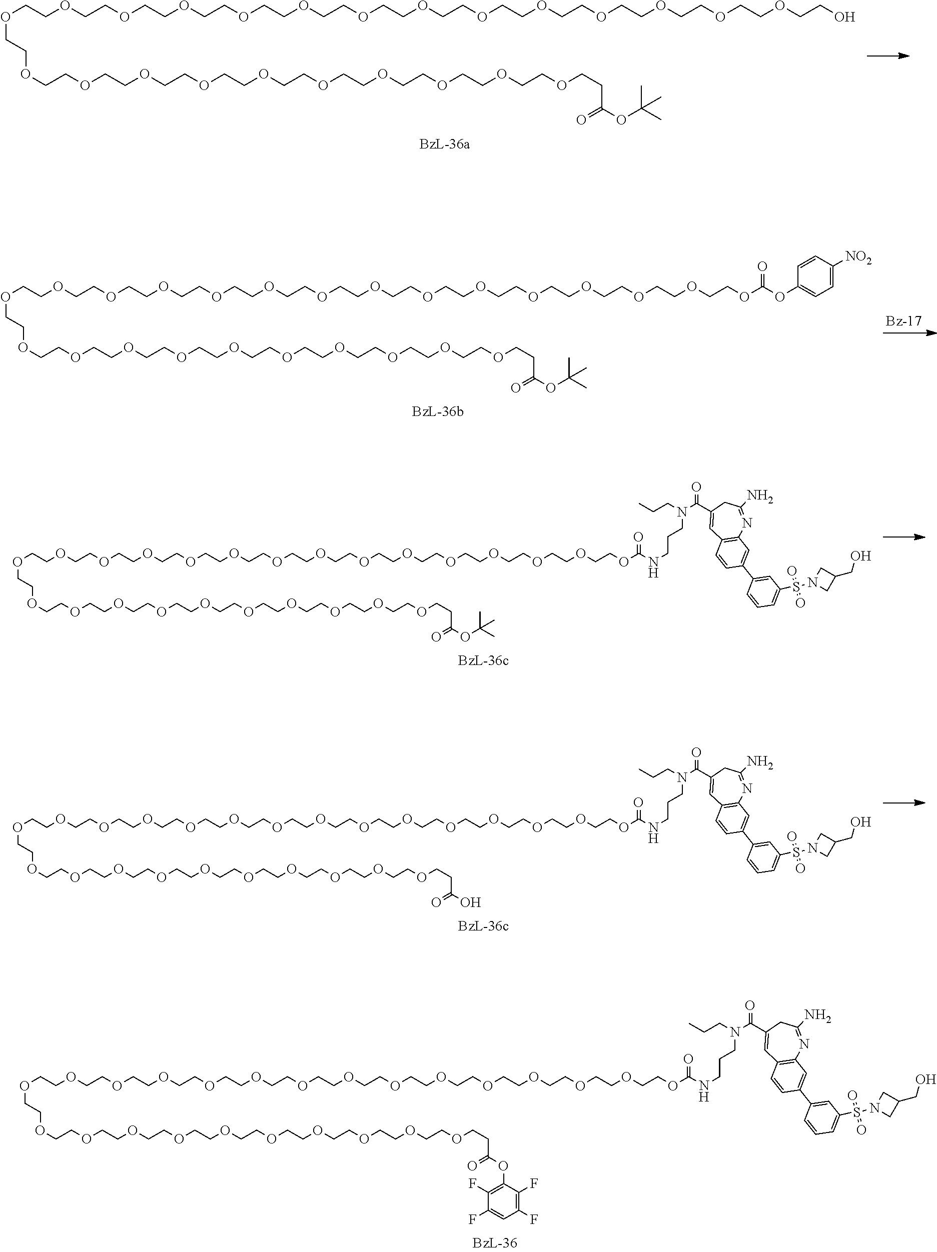

Preparation of BzL-36b: A vial was charged with tert-butyl 1-hydroxy-3,6,9,12,15,18,21,24,27,30,33,36,39,42,45,48,51,54,57,60,63,66,69,72-tetracosaoxapentaheptacontan-75-oate, BzL-36a (148 mg, 0.123 mmol), diisopropylethylamine (0.369 mmol) and 0.6 mL anhydrous DMF. The vial was cooled to 0° C., then 4-nitrophenylchloroformate (0.123 mmol) was added portion-wise. The reaction was warmed to room temperature and maintained for 3 h, then purified by reverse phase preparative HPLC utilizing a 25-75% gradient of acetonitrile:water containing 0.1% trifluoroacetic acid. The purified fractions were combined and lyophilized to afford 42.5 mg of tert-butyl 1-(4-nitrophenoxy)-1-oxo-2,5,8,11,14,17,20,23,26,29,32,35,38,41,44,47,50,53,56,59,62,65,68,71,74-pentacosaoxaheptaheptacontan-77-oate, BzL-36b. LC/MS [M+H]1368.7 (calculated); LC/MS [M+H]1368.7 (observed).

Preparation of BzL-36c: A vial was charged with Bz-17 (0.0275 mmol), BzL-36b (0.0275 mmol), HOAT (0.02 mmol), diisopropylethylamine (0.0825 mmol), 250 μL DCM, and 250 μL DMF. The reaction was maintained until all starting material was consumed by LCMS. The crude reaction was purified by reverse phase preparative HPLC utilizing a 25-75% gradient of acetonitrile:water containing 0.1% trifluoroacetic acid. The purified fractions were combined and lyophilized to afford 22.5 mg of tert-butyl 82-(2-amino-8-(3-((3-(hydroxymethyl)azetidin-1-yl)sulfonyl)phenyl)-3H-benzo[b]azepine-4-carbonyl)-77-oxo-4,7,10,13,16,19,22,25,28,31,34,37,40,43,46,49,52,55,58,61,64,67,70,73,76-pentacosaoxa-78,82-diazapentaoctacontanoate, BzL-36c. LC/MS [M+H]1754.9 (calculated); LC/MS [M+H]1754.9 (observed).

Preparation of BzL-36d: A vial was charged with BzL-36c (0.0128 mmol), 1 mL DCM, and 0.2 mL trifluoroacetic acid. The reaction was maintained for 3 h, then concentrated under reduced pressure. The resultant residue was azeotroped thrice with toluene to give 82-(2-amino-8-(3-((3-(hydroxymethyl)azetidin-1-yl)sulfonyl)phenyl)-3H-benzo[b]azepine-4-carbonyl)-77-oxo-4,7,10,13,16,19,22,25,28,31,34,37,40,43,46,49,52,55,58,61,64,67,70,73,76-pentacosaoxa-78,82-diazapentaoctacontanoic acid, BzL-36d which was used immediately in the subsequent step.

Preparation of BzL-36: A vial was charged with BzL-36d (8.9 mg, 0.005 mmol), 2,3,5,6-tetrafluorophenol (1.8 mg, 0.011 mmol), collidine (2.2 μL, 0.016 mmol), 1-ethyl-3-(3-dimethylaminopropyl)carbodiimide (1 mg, 0.005 mmol) and 100 μL DMF. The reaction was stirred for 6 h, then purified by reverse phase preparative HPLC utilizing a 25-75% gradient of acetonitrile:water containing 0.1% trifluoroacetic acid. The purified fractions were combined and lyophilized to afford 6.3 mg of 2,3,5,6-tetrafluorophenyl 82-(2-amino-8-(3-((3-(hydroxymethyl)azetidin-1-yl)sulfonyl)phenyl)-3H-benzo[b]azepine-4-carbonyl)-77-oxo-4,7,10,13,16,19,22,25,28,31,34,37,40,43,46,49,52,55,58,61,64,67,70,73,76-pentacosaoxa-78,82-diazapentaoctacontanoate, BzL-36. LC/MS [M+H]1846.9 (calculated); LC/MS [M+H]1846.9 (observed).

### Example 57 Synthesis of BzL-37

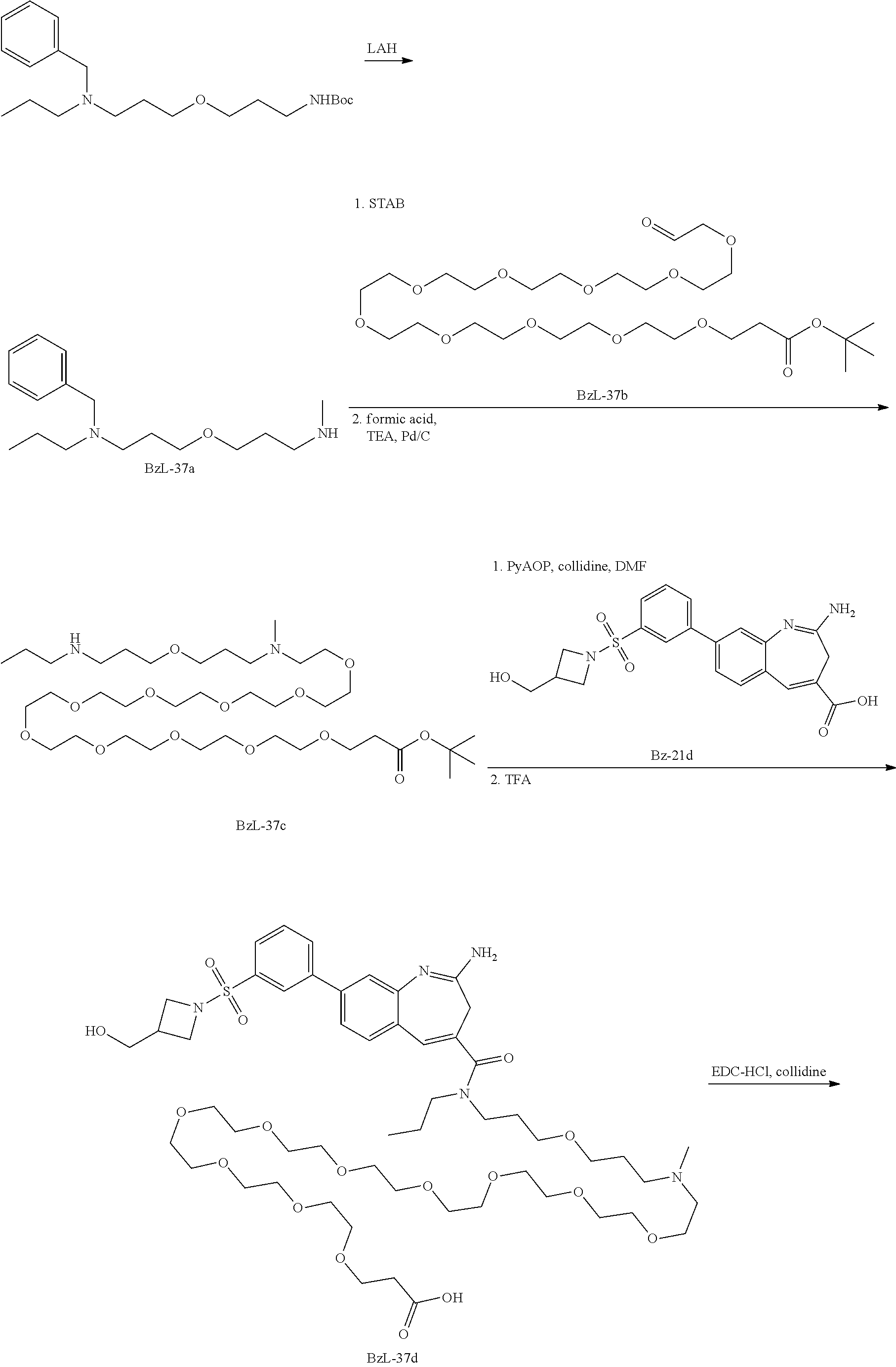 [SMILES: CCCN(CCCOCCCNC(=O)OC(C)(C)C)Cc1ccccc1 ; CCCN(CCCOCCCNC)Cc1ccccc1 ; CC(C)(C)OC(=O)CCOCCOCCOCCOCCOCCOCCOCCOCCOCCOCC=O ; CCCNCCCOCCCN(C)CCOCCOCCOCCOCCOCCOCCOCCOCCOCCOCCC(=O)OC(C)(C)C ; NC1=Nc2cc(-c3cccc(S(=O)(=O)N4CC(CO)C4)c3)ccc2C=C(C(=O)O)C1 ; CCCN(CCCOCCCN(C)CCOCCOCCOCCOCCOCCOCCOCCOCCOCCOCCC(=O)O)C(=O)C1=Cc2ccc(-c3cccc(S(=O)(=O)N4CC(CO)C4)c3)cc2N=C(N)C1]

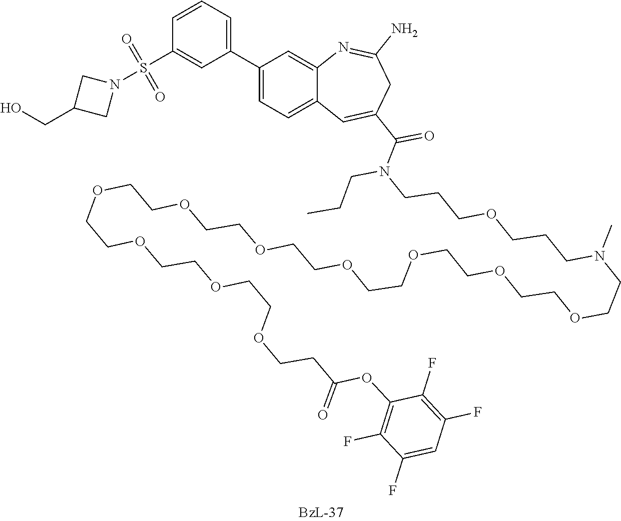 [SMILES: CCCN(CCCOCCCN(C)CCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CO)C4)c3)cc2N=C(N)C1]

Preparation of BzL-37a: tert-Butyl (3-(3-(benzyl(propyl)amino)propoxy)propyl)carbamate (0.032 g, 0.088 mmol, 1 eq.) was dissolved in THF. Lithium aluminum hydride (0.01 g, 0.26 mmol, 3 eq.) was added and the reaction heated to 60° C. The reaction was concentrated and purified by HPLC to give N-benzyl-3-(3-(methylamino)propoxy)-N-propylpropan-1-amine, BzL-37a (0.01 g, 0.036 mmol, 41%). LC/MS [M+H]279.24 (calculated); LC/MS [M+H]279.33 (observed).

Preparation of BzL-37c: BzL-37a (0.01 g, 0.036 mmol, 1 eq.) and tert-butyl 1-oxo-3,6,9,12,15,18,21,24,27,30-decaoxatritriacontan-33-oate, BzL-37b (0.02 g, 0.036 mmol, 1 eq.) were dissolved in DCM. Sodium triacetoxyborohydride, STAB (0.022 g, 0.11 mmol, 3 eq.) was added and the reaction stirred at room temperature. The solution was concentrated and purified by HPLC. The purified product was taken up in methanol with triethylamine. Formic acid was added, followed by 10 wt % Pd/C, and the reaction heated to 60° C. Upon consumption of starting material, the reaction mixture was filtered and concentrated to give tert-butyl 34-methyl-4,7,10,13,16,19,22,25,28,31,38-undecaoxa-34,42-diazapentatetracontanoate, BzL-37c (0.007 g, 0.0092 mmol, 26%). LC/MS [M+H]757.74 (calculated); LC/MS [M+H]757.85 (observed).

Preparation of BzL-37d: 2-Amino-8-(3-((3-(hydroxymethyl)azetidin-1-yl)sulfonyl)phenyl)-3H-benzo[b]azepine-4-carboxylic acid, Bz-21d (0.0040 g, 0.0092 mmol, 1 eq.), BzL-37c (0.007 g, 0.0092 mmol, 1 eq.), and collidine (0.004 ml, 0.028 mmol, 3 eq.) were dissolved in DMF. PyAOP (0.0072 g, 0.014 mmol, 1.5 eq.) was added and the mixture stirred at room temperature. When complete, the reaction mixture was concentrated and purified by RP-HPLC. The isolated product was concentrated, dissolved in minimal TFA, and allowed to stand at room temperature for 15 minutes. The solution was then concentrated and purified by RP-HPLC to give 42-(2-amino-8-(3-((3-(hydroxymethyl)azetidin-1-yl)sulfonyl)phenyl)-3H-benzo[b]azepine-4-carbonyl)-34-methyl-4,7,10,13,16,19,22,25,28,31,38-undecaoxa-34,42-diazapentatetracontanoic acid, BzL-37d (0.004 g, 0.0036 mmol, 39%). LC/MS [M+H]1110.59 (calculated); LC/MS [M+H]1110.93 (observed).

Preparation of BzL-37: BzL-37d (0.004 g, 0.0036 mmol, 1 eq.) and TFP (0.0033 g, 0.018 mmol, 5 eq.) were dissolved in 1 ml DMF. Collidine (0.005 ml, 0.036 mmol, 10 eq.) was added, followed by EDC-HCl (0.0035 g, 0.018 mmol, 5 eq.). The reaction was stirred at room temperature and monitored by LCMS, then concentrated and purified by HPLC to give 2,3,5,6-tetrafluorophenyl 42-(2-amino-8-(3-((3-(hydroxymethyl)azetidin-1-yl)sulfonyl)phenyl)-3H-benzo[b]azepine-4-carbonyl)-34-methyl-4,7,10,13,16,19,22,25,28,31,38-undecaoxa-34,42-diazapentatetracontanoate, BzL-37 (0.0016 g, 0.0013 mmol, 35%). LC/MS [M+H]1258.58 (calculated); LC/MS [M+H]1258.96 (observed).

### Example 58 Synthesis of BzL-38

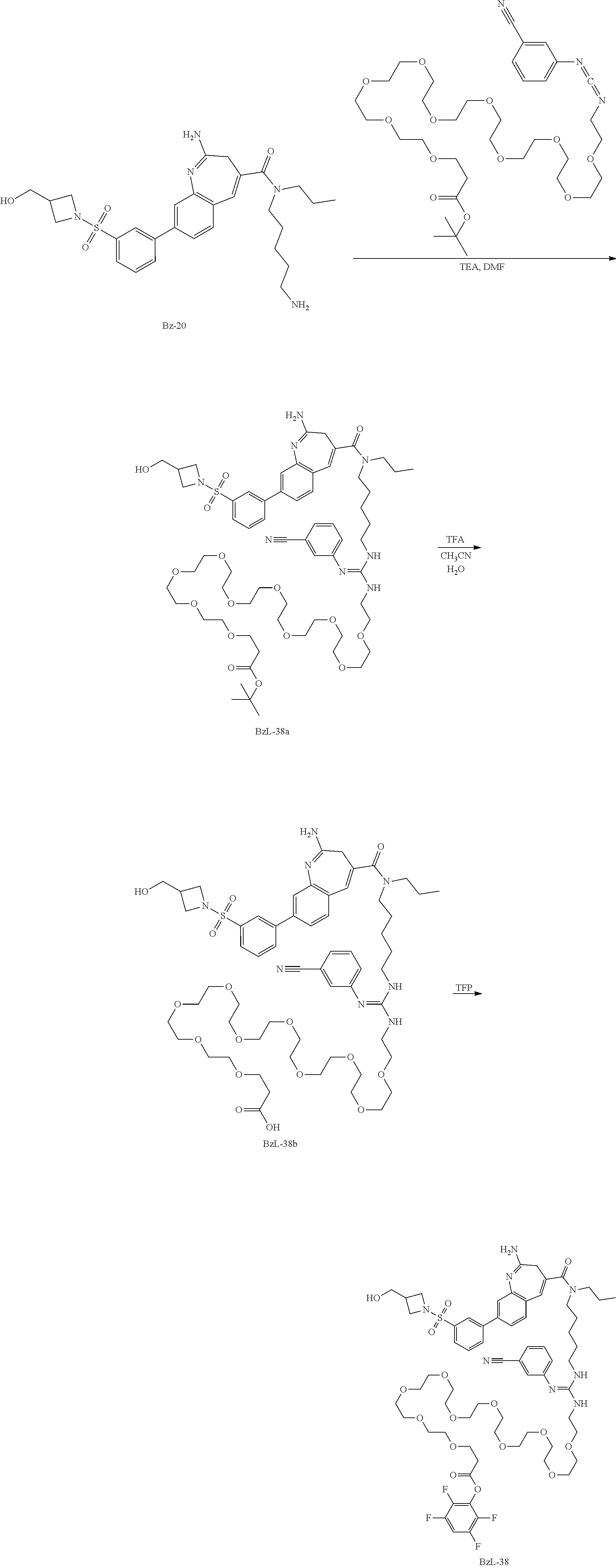

Preparation of BzL-38a: This was prepared using the same methods as described in the synthesis of BzL-42. LC/MS [M+H]1265.7 (calculated); LC/MS [M+H]1265.7 (observed).

Preparation of BzL-38b: This was prepared using the same method as described in the synthesis of BzL-42. LC/MS [M+H]1209.6 (calculated); LC/MS [M+H]1209.6 (observed).

Preparation of BzL-38: This was prepared using the same method as described in the synthesis of BzL-42. LC/MS [M+H]1357.6 (calculated); LC/MS [M+H]1357.6 (observed).

### Example 59 Synthesis of BzL-39

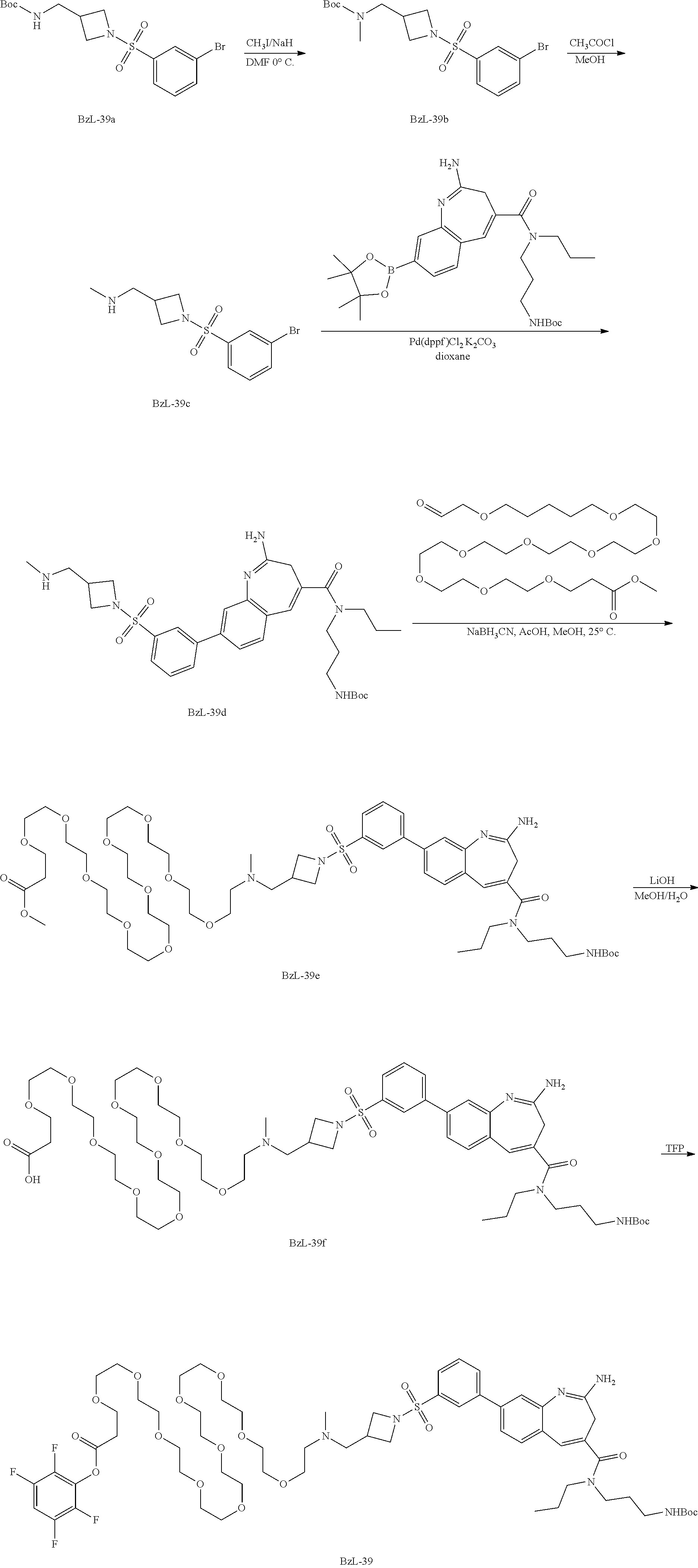

Preparation of BzL-39b: To a solution of tert-butyl N-[[1-(3-bromophenyl)sulfonylazetidin-3-yl]methyl]carbamate, BzL-39a (1.0 g, 2.47 mmol, 1.0 eq) in DMF (10 mL) was added sodium hydride, NaH (148 mg, 3.70 mmol, 60% purity, 1.5 eq) in portions and it was stirred at 0° C. for 0.5 h. Then methyl iodide, CH₃I (1.05 g, 7.40 mmol, 461 μL, 3.0 eq) was added and then stirred at 25° C. for 1 h. The reaction was quenched with water and extracted with EtOAc (30 mL×3). The organic layer was washed with brine, dried over Na₂SO₄, filtered and concentrated to give tert-butyl N-[[1-(3-bromophenyl) sulfonylazetidin-3-yl]methyl]-N-methyl-carbamate, BzL-39b (1.3 g, crude) as yellow oil. ¹H NMR (CDCl₃, 400 MHz) δ7.99 (t, J=2.0 Hz, 1H), 7.80-7.75 (m, 2H), 7.47 (t, J=8.0 Hz, 1H), 3.85 (t, J=7.6 Hz, 2H), 3.57 (t, J=7.2 Hz, 2H), 3.29 (d, J=7.2 Hz, 2H), 2.75 (s, 3H), 2.74-2.70 (m, 1H), 1.43 (s, 9H), 1.26 (t, J=7.2 Hz, 3H).

Preparation of BzL-39c: To a solution of BzL-39b (1.3 g, 3.10 mmol, 1.0 eq) in MeOH (20 mL) was added acetyl chloride (1.22 g, 15.5 mmol, 1.11 mL, 5.0 eq) at 25° C. and it was stirred at 50° C. for 1 h. Then the mixture was concentrated to give 1-[1-(3-bromophenyl)sulfonylazetidin-3-yl]-N-methyl-methanamine, BzL-39c (1 g, crude) as white solid. ¹H NMR (MeOD, 400 MHz) δ 8.00-7.98 (m, 1H), 7.93 (d, J=8.0 Hz, 1H), 7.84 (d, J=8.0 Hz, 1H), 7.64-7.59 (m, 1H), 3.94 (t, J=8.4 Hz, 2H), 3.64 (dd, J=5.6, 8.4 Hz, 2H), 3.14 (d, J=7.6 Hz, 2H), 2.84-2.77 (m, 1H), 2.66 (s, 3H).

Preparation of BzL-39d: To a mixture of tert-butyl N-[3-[[2-amino-8-(4,4,5,5-tetramethyl-1,3,2-dioxaborolan-2-yl)-3H-1-benzazepine-4-carbonyl]-propyl-amino]propyl]carbamate (0.44 g, 835 umol, 1.0 eq) and Bzl-39c (357 mg, 1.00 mmol, 1.2 eq, HCl) in dioxane (4 mL) and H₂O (0.5 mL) was added Pd(dppf)C₁₂ (30.6 mg, 41.79 umol, 0.05 eq) and K₂CO₃ (231.0 mg, 1.67 mmol, 2.0 eq) at 15° C. under N₂. The mixture was stirred at 90° C. for 3 hours. The reaction was cooled to 15° C. and then filtered. The filtrate was poured into ice water (30 mL) and stirred for 5 min. The aqueous phase was extracted with ethyl acetate (20 mL×3) and combined organic phase was washed with brine (20 mL), dried with anhydrous Na₂SO₄, filtered and concentrated in vacuum. The residue was purified by flash silica gel chromatography (ISCO®; 40 g SepaFlash® Silica Flash Column, Eluent of 0-100% Ethyl acetate/Petroleum ether to EtOAc/MeOH=3/1 gradient @60 mL/min) to afford tert-butyl N-[3-[[2-amino-8-[3-[3-(methylaminomethyl)azetidin-1-yl]sulfonylphenyl]-3H-1-benzazepine-4-carbonyl]-propyl-amino]propyl]carbamate, BzL-39d (0.32 g, 500.92 umol, 59.94% yield) as yellow solid.

Preparation of BzL-39e: To a mixture of BzL-39d (0.2 g, 313 umol, 1.0 eq) and methyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (170 mg, 313 umol, 1.0 eq) in MeOH (20 mL) was added acetic acid, AcOH (94.0 mg, 1.57 mmol, 5.0 eq) at 25° C. The mixture was stirred at this temperature for 10 min, then sodium cyanoborohydride, NaBH₃CN (39.3 mg, 626. umol, 2.0 eq) was added and the mixture was stirred at 25° C. for 18 hours. The reaction mixture was concentrated to give the crude product methyl 3-[2-[2-[2-[2-[2-[2-[2-[2-[2-[2-[[1-[3-[2-amino-4-[3-(tert-butoxycarbonylamino)propyl-propyl-carbamoyl]-3H-1-benzazepin-8-yl]phenyl]sulfonylazetidin-3-yl]methyl-methyl-amino]ethoxy]ethoxy]ethoxy]ethoxy]ethoxy]ethoxy]ethoxy]ethoxy]ethoxy]ethoxy]propanoate, BzL-39e (0.36 g, crude).

Preparation of BzL-39f: To a mixture of BzL-39e (0.36 g, 308 umol, 1.0 eq) in MeOH (20 mL) was added a solution of lithium hydroxide hydrate, LiOH·H₂O (130 mg, 3.09 mmol, 10.0 eq) in H₂O (2 mL) at 25° C. and then stirred at 25° C. for 18 hours. The reaction mixture was quenched with aq HCl (4 M) until pH=7 and concentrated at 40° C. The residue was purified by prep-HPLC (column: Welch Xtimate C18 100*25 mm*3 um; mobile phase: [water (0.1% TFA) -ACN]; B %: 20%-40%, 12 min) to give 3-[2-[2-[2-[2-[2-[2-[2-[2-[2-[2-[[1-[3-[2-amino-4-[3-(tert-butoxycarbonylamino)propyl-propyl-carbamoyl]-3H-1-benzazepin-8-yl]phenyl]sulfonylazetidin-3-yl]methyl-methyl-amino]ethoxy]ethoxy]ethoxy]ethoxy]ethoxy]ethoxy]ethoxy]ethoxy]ethoxy]ethoxy]propanoic acid, BzL-39f (56 mg, 48.64 umol, 16% yield) as light yellow oil. ¹H NMR (MeOD, 400 MHz) δ8.19-8.03 (m, 2H), 7.97-7.88 (m, 1H), 7.88-7.82 (m, 1H), 7.82-7.76 (m, 2H), 7.73-7.71 (m, 1H), 7.13 (s, 1H), 4.02 (t, J=8.0 Hz, 2H), 3.79 (t, J=4.8 Hz, 2H), 3.75-3.68 (m, 4H), 3.64-3.45 (m, 42H), 3.38 (s, 2H), 3.17-2.94 (m, 4H), 2.86 (s, 3H), 2.53 (t, J=6.4 Hz, 2H), 1.89-1.80 (m, 2H), 1.75-1.63 (m, 2H), 1.47-1.42 (m, 9H), 1.02-0.86 (m, 3H). LC/MS [M+H]1151.61 (calculated); LC/MS [M+2H/2]576.5 (observed).

Preparation of BzL-39. BzL-39f (0.056 g, 0.049 mmol, 1 eq.) and TFP (0.040 g, 0.24 mmol, 5 eq.) were dissolved in 2 ml DMF. Collidine (0.064 ml, 0.49 mmol, 10 eq.) was added, followed by EDC-HCl (0.047 g, 0.24 mmol, 5 eq.). The reaction was stirred at room temperature and monitored by LCMS, then concentrated and purified by HPLC to give 2,3,5,6-tetrafluorophenyl 1-(1-((3-(2-amino-4-((3-((tert-butoxycarbonyl)amino)propyl)(propyl)carbamoyl)-3H-benzo[b]azepin-8-yl)phenyl)sulfonyl)azetidin-3-yl)-2-methyl-5,8,11,14,17,20,23,26,29,32-decaoxa-2-azapentatriacontan-35-oate, BzL-39 (0.027 g, 0.021 mmol, 42%). LC/MS [M+H]1299.61 (calculated); LC/MS [M+H]1300.00 (observed).

### Example 60 Synthesis of BzL-40

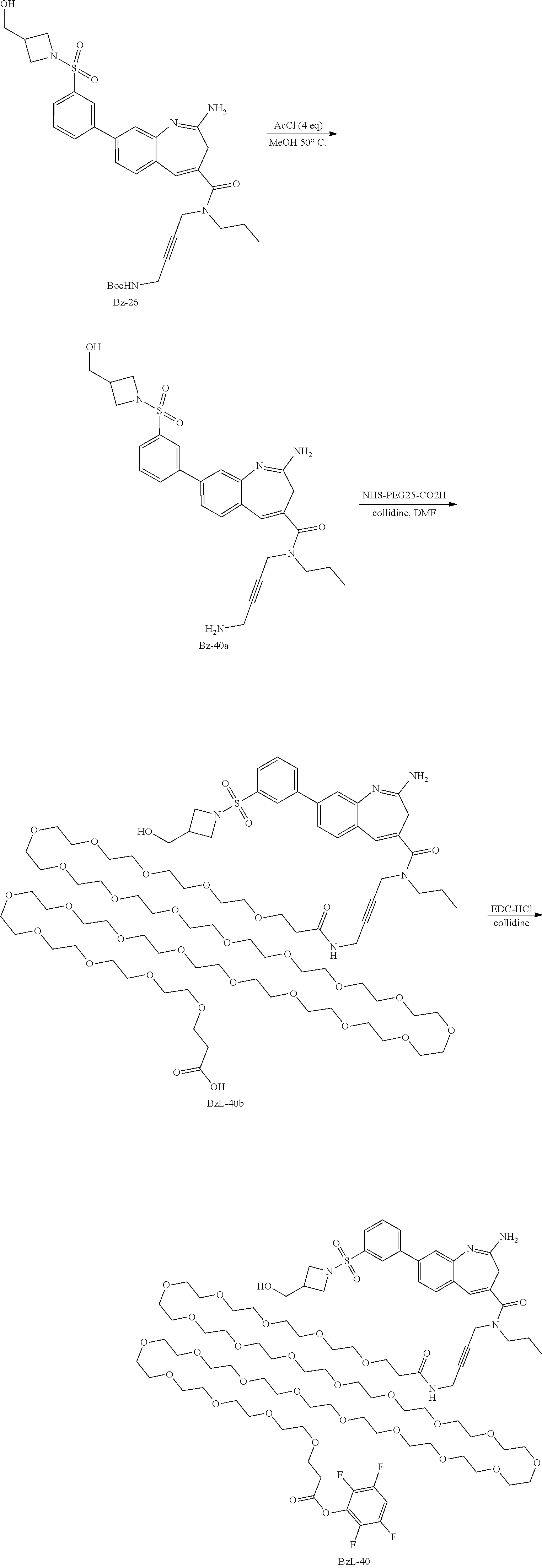

Preparation of BzL-40a: To a mixture of tert-butyl N-[4-[[2-amino-8-[3-[3-(hydroxymethyl)azetidin-1-yl]sulfonylphenyl]-3H-1-benzazepine-4-carbonyl]-propyl-amino]but-2-ynyl]carbamate, Bz-26 (800 mg, 1.26 mmol, 1.0 eq) in MeOH (20 mL) was added acetyl chloride (395 mg, 5.03 mmol, 360 μL, 4.0 eq) at 25° C. under N₂ and then stirred at 50° C. for 1 hour. The mixture was quenched with solid NaHCO₃ until pH to ˜8, then filtered and concentrated in vacuum. The residue was purified by prep-HPLC (column: Phenomenex Luna C18 200*40 mm*10 um; mobile phase: [water(10 mM NH₄HCO₃)-ACN]; B %: 10%-40%, 10 min) to afford 2-amino-N-(4-aminobut-2-ynyl)-8-[3-[3-(hydroxymethyl)azetidin-1-yl]sulfonylphenyl]-N-propyl-3H-1-benzazepine-4-carboxamide, BzL-40a (220 mg, 411 umol, 32.6% yield) as white solid. ¹H NMR (MeOD, 400 MHz) δ8.12-8.01 (m, 2H), 7.90-7.82 (m, 1H), 7.80-7.72 (m, 1H), 7.56-7.47 (m, 2H), 7.44-7.38 (m, 1H), 7.15 (s, 1H), 4.32 (s, 2H), 3.86 (t, J=8.0 Hz, 2H), 3.69-3.47 (m, 6H), 3.41 (d, J=6.4 Hz, 2H), 2.64-2.51 (m, 1H), 1.84-1.63 (m, 2H), 0.99-0.91 (m, 3H). LC/MS [M+H]536.2 (calculated); LC/MS [M+H]536.3 (observed). Preparation of BzL-40b: BzL-40a (0.045 g, 0.084 mmol, 1 eq.) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid, NHS-PEG25-CO₂H (0.11 g, 0.084 mmol, 1 eq.) were dissolved in DMF, followed by collidine (0.054 ml, 0.42 mmol, 5 eq.). The reaction was purified by HPLC to give 85-(2-amino-8-(3-((3-(hydroxymethyl)azetidin-1-yl)sulfonyl)phenyl)-3H-benzo[b]azepine-4-carbonyl)-79-oxo-4,7,10,13,16,19,22,25,28,31,34,37,40,43,46,49,52,55,58,61,64,67,70,73,76-pentacosaoxa-80,85-diazaoctaoctacont-82-ynoic acid, BzL-40b (0.1 g, 0.0058 mmol, 69%). LC/MS [M+H]1736.90 (calculated); LC/MS [M+H]1737.32 (observed).

Preparation of BzL-40: BzL-40b (0.1 g, 0.0058 mmol, 1 eq.) and TFP (0.014 g, 0.086 mmol, 1.5 eq.) were dissolved in DMF. Collidine (0.038 ml, 0.29 mmol, 5 eq.) was added, followed by EDC-HCl (0.022 g, 0.115 mmol, 2 eq.). The reaction was stirred at room temperature and monitored by LCMS, then concentrated and purified by HPLC to give 2,3,5,6-tetrafluorophenyl 85-(2-amino-8-(3-((3-(hydroxymethyl)azetidin-1-yl)sulfonyl)phenyl)-3H-benzo[b]azepine-4-carbonyl)-79-oxo-4,7,10,13,16,19,22,25,28,31,34,37,40,43,46,49,52,55,58,61,64,67,70,73,76-pentacosaoxa-80,85-diazaoctaoctacont-82-ynoate, BzL-40 (0.014 g, 0.0076 mmol, 13%). LC/MS [M+H]1884.90 (calculated); LC/MS [M+H]1885.44 (observed).

### Example 61 Synthesis of BzL-41

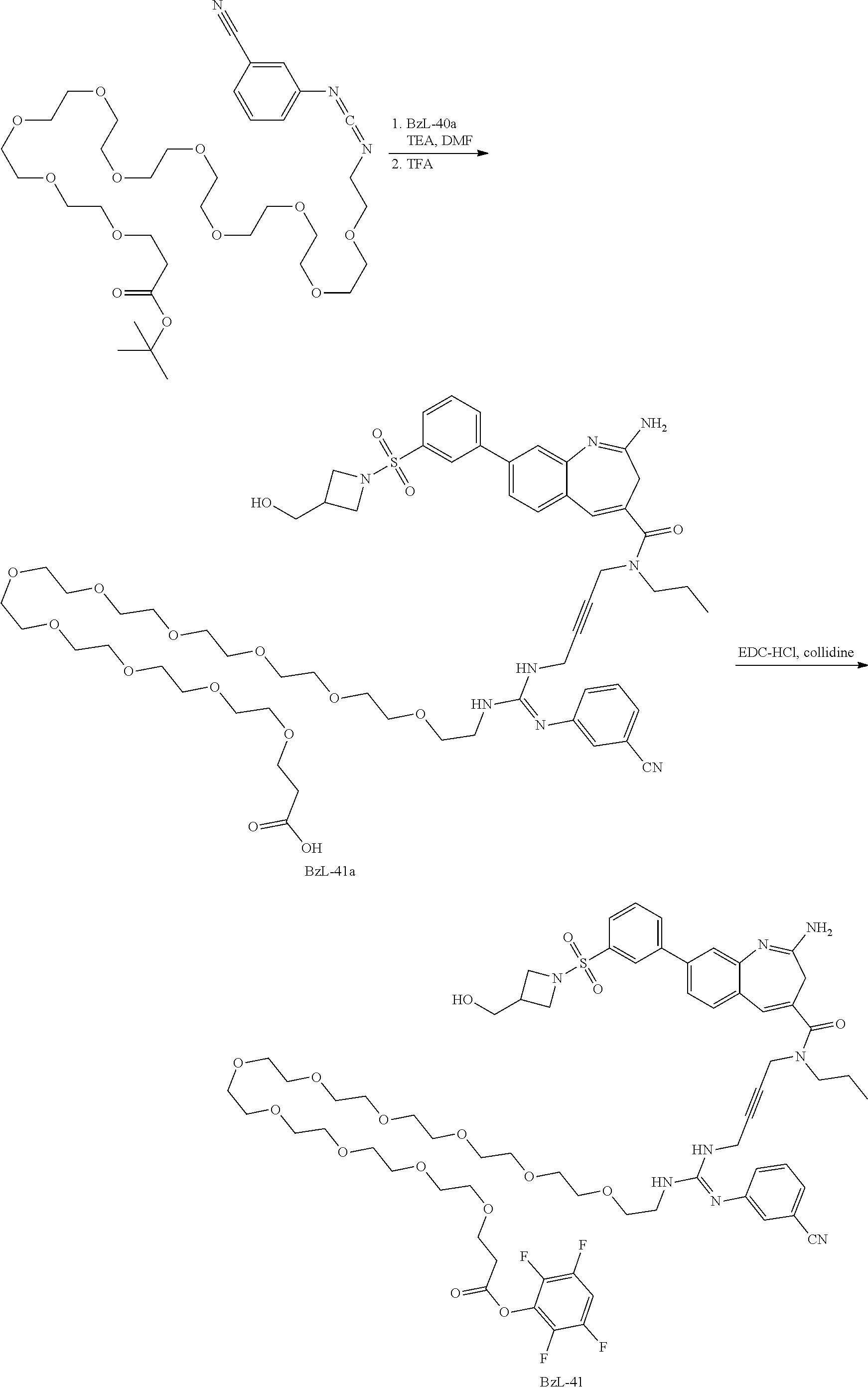 [SMILES: CC(C)(C)OC(=O)CCOCCOCCOCCOCCOCCOCCOCCOCCOCCOCCN=C=Nc1cccc(C#N)c1 ; CCCN(CC#CCN/C(=N\c1cccc(C#N)c1)NCCOCCOCCOCCOCCOCCOCCOCCOCCOCCOCCC(=O)O)C(=O)C1=Cc2ccc(-c3cccc(S(=O)(=O)N4CC(CO)C4)c3)cc2N=C(N)C1 ; CCCN(CC#CCN/C(=N\c1cccc(C#N)c1)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CO)C4)c3)cc2N=C(N)C1]

Preparation of BzL-41a: 2-Amino-N-(4-aminobut-2-yn-1-yl)-8-(3-((3-(hydroxymethyl)azetidin-1-yl)sulfonyl)phenyl)-N-propyl-3H-benzo[b]azepine-4-carboxamide, BzL-40a (0.05 g, 0.093 mmol, 1 eq.) and tert-butyl 1-((3-cyanophenyl)imino)-5,8,11,14,17,20,23,26,29,32-decaoxa-2-azapentatriacont-1-en-35-oate (0.066 g, 0.093 mmol, 1 eq.) were dissolved in DMF. Triethylamine (0.05 ml, 0.36 mmol, 3.8 eq.) was added, and the reaction was stirred at ambient temperature. Upon consumption of amine starting material, the reaction was concentrated and purified by HPLC. The isolated t-butyl ester product was taken up in minimal TFA for 10 minutes, then concentrated to give 41-(2-amino-8-(3-((3-(hydroxymethyl)azetidin-1-yl)sulfonyl)phenyl)-3H-benzo[b]azepine-4-carbonyl)-35-((3-cyanophenyl)imino)-4,7,10,13,16,19,22,25,28,31-decaoxa-34,36,41-triazatetratetracont-38-ynoic acid, BzL-41a (0.05 g, 0.042 mmol, 45%). LC/MS [M+H]1191.56 (calculated); LC/MS [M+H]1192.00 (observed).

Preparation of BzL-41: BzL-41a (0.05 g, 0.042 mmol, 1 eq.) and TFP (0.01 g, 0.063 mmol, 1.5 eq.) were dissolved in DMF. Collidine (0.028 ml, 0.21 mmol, 5 eq.) was added, followed by EDC-HCl (0.016 g, 0.084 mmol, 2 eq.). The reaction was stirred at room temperature and monitored by LCMS, then concentrated and purified by HPLC to give 2,3,5,6-tetrafluorophenyl 41-(2-amino-8-(3-((3-(hydroxymethyl)azetidin-1-yl)sulfonyl)phenyl)-3I-benzo[I]azepine-4-carbonyl)-35-((3-cyanophenyl)imino)-4,7,10,13,16,19,22,25,28,31-decaoxa-34,36,41-triazatetratetracont-38-ynoate, BzL-41 (0.019 g, 0.014 mmol, 35%). LC/MS [M+H]1339.56 (calculated); LC/MS [M+H]1340.04 (observed).

### Example 62 Synthesis of BzL-42

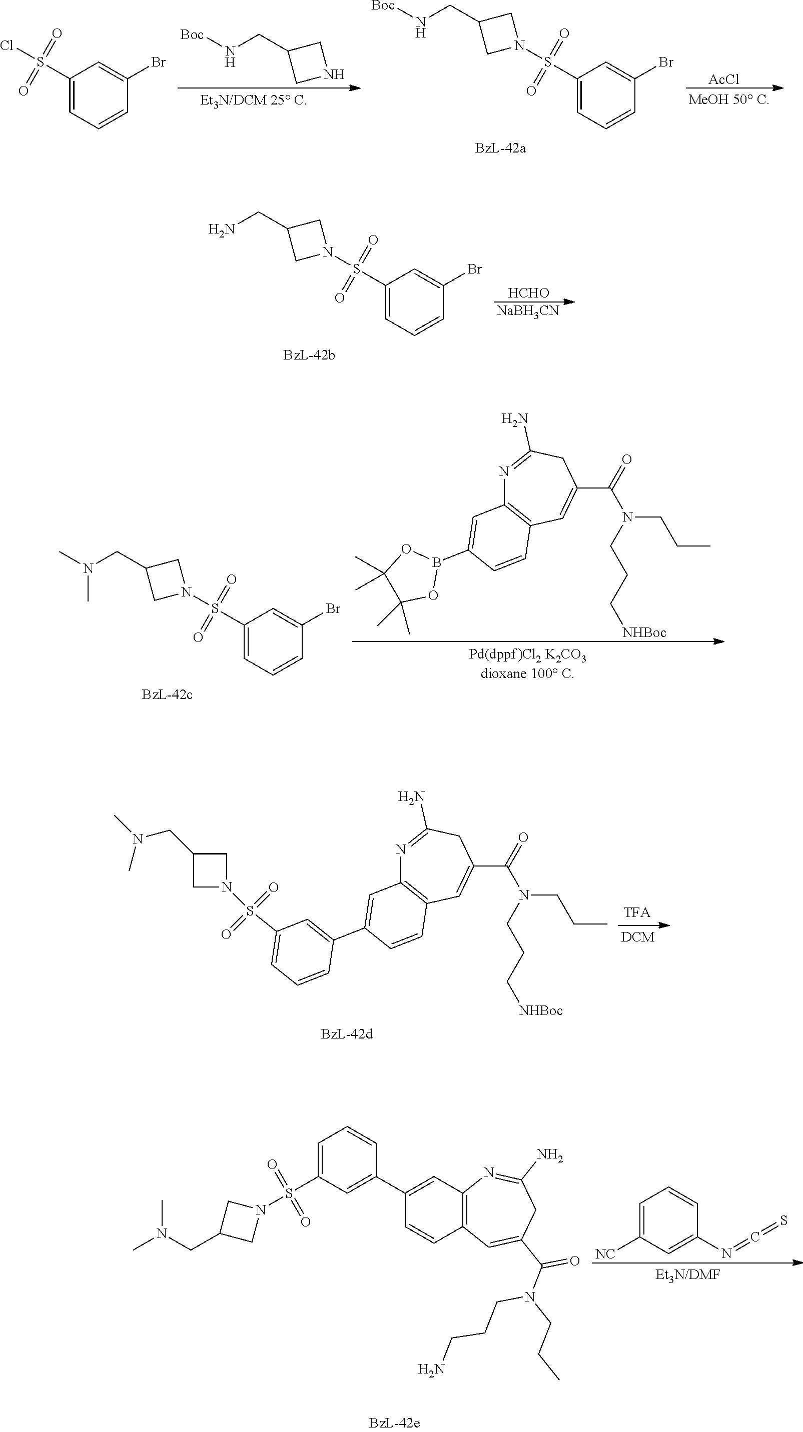

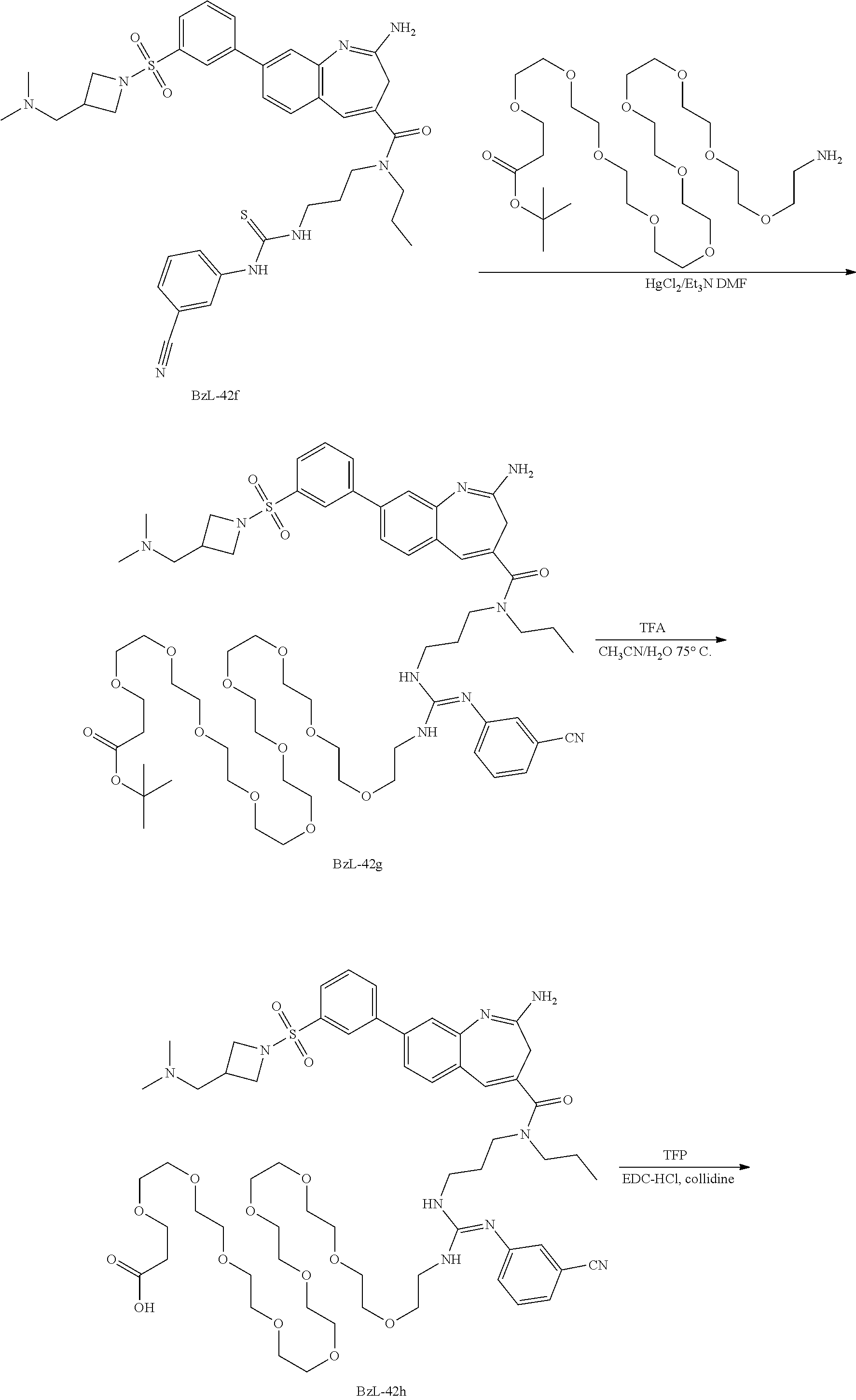 [SMILES: CCCN(CCCNC(=S)Nc1cccc(C#N)c1)C(=O)C1=Cc2ccc(-c3cccc(S(=O)(=O)N4CC(CN(C)C)C4)c3)cc2N=C(N)C1 ; CC(C)(C)OC(=O)CCOCCOCCOCCOCCOCCOCCOCCOCCOCCOCCN ; CCCN(CCCN/C(=N/c1cccc(C#N)c1)NCCOCCOCCOCCOCCOCCOCCOCCOCCOCCOCCC(=O)OC(C)(C)C)C(=O)C1=Cc2ccc(-c3cccc(S(=O)(=O)N4CC(CN(C)C)C4)c3)cc2N=C(N)C1 ; CCCN(CCCN/C(=N/c1cccc(C#N)c1)NCCOCCOCCOCCOCCOCCOCCOCCOCCOCCOCCC(=O)O)C(=O)C1=Cc2ccc(-c3cccc(S(=O)(=O)N4CC(CN(C)C)C4)c3)cc2N=C(N)C1 ; "TFP"]

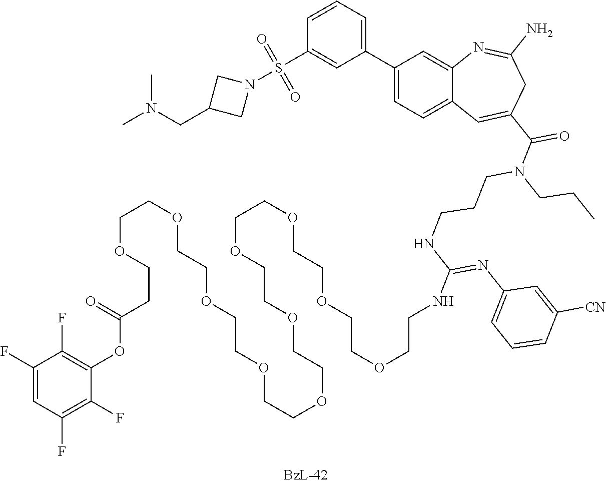 [SMILES: CCCN(CCCN/C(=N/c1cccc(C#N)c1)NCCOCCOCCOCCOCCOCCOCCOCCOCCOCCOCCC(=O)Oc1c(F)c(F)cc(F)c1F)C(=O)C1=Cc2ccc(-c3cccc(S(=O)(=O)N4CC(CN(C)C)C4)c3)cc2N=C(N)C1]

Preparation of BzL-42a: To a mixture of 3-bromobenzenesulfonyl chloride (8.23 g, 32.2 mmol, 4.65 mL, 1.0 eq) and tert-butyl N-(azetidin-3-ylmethyl)carbamate (6.0 g, 32.2 mmol, 1.0 eq) in DCM (100 mL) was added Et₃N (6.52 g, 64.4 mmol, 8.97 mL, 2.0 eq) at 0° C. and then stirred at this temperature for 1 h. The reaction was diluted with water and extracted with EtOAc (50 mL×3). The organic layer was washed with brine, dried over Na₂SO₄, filtered and concentrated to afford tert-butyl N-[[1-(3-bromophenyl)ulfonylazetidin-3-yl]methyl]carbamate, BzL-42a (12 g, crude) as white solid. ¹H NMR (CDCl₃, 400 MHz) δ7.99 (t, J=1.6 Hz, 1H), 7.78 (m, 2H), 7.47 (t, J=8.0 Hz, 1H), 4.63 (s, 1H), 3.85 (t, J=8.0 Hz, 2H), 3.54 (dd, J=5.6, 8.0 Hz, 2H), 3.21-3.16 (m, 2H), 2.67-2.62 (m, 1H), 1.42 (s, 9H). LC/MS [M+Na]427.0 (calculated); LC/MS [M+Na]427.0 (observed).

Preparation of BzL-42b: To a mixture of BzL-42a (2 g, 4.93 mmol, 1.0 eq) in MeOH (30 mL) was added acetyl chloride (1.94 g, 24.67 mmol, 1.76 mL, 5.0 eq) at 25° C. and then stirred at this temperature for 2 h. The mixture was concentrated to give [1-(3-bromophenyl)sulfonylazetidin-3-yl]methanamine, BzL-42b (1.5 g, crude) as white solid. ¹H NMR (MeOD, 400 MHz) δ7.99 (t, J=1.6 Hz, 1H), 7.93 (d, J=8.0 Hz, 1H), 7.84 (d, J=7.2 Hz, 1H), 7.62 (t, J=8.0 Hz, 1H), 3.93 (t, J=8.4 Hz, 2H), 3.61 (m, 2H), 3.06-3.03 (m, 2H), 2.78-2.66 (m, 1H).

Preparation of BzL-42c: To a mixture of BzL-42b (4.0 g, 13.1 mmol, 1.0 eq) in MeOH (40 mL) was added Et₃N (1.99 g, 19.7 mmol, 2.74 mL, 1.5 eq), formaldehyde (4.25 g, 52.4 mmol, 3.90 mL, 37% purity, 4.0 eq) and NaBH₃CN (1.65 g, 26.2 mmol, 2.0 eq) at 25° C. and it was stirred at 25° C. for 2 h. The mixture was diluted with water and extracted with EtOAc (30 mL×3). The organic layer was washed with brine, dried over Na₂SO₄, filtered and concentrated. The residue was purified by silica gel chromatography (column height: 250 mm, diameter: 100 mm, 100-200 mesh silica gel, EtOAc(1.5% NH₃—H₂O):MeOH═I/O, 1/1) to afford 1-[1-(3-bromophenyl) sulfonylazetidin-3-yl]-N,N-dimethyl-methanamine, BzL-42c (1.6 g, 4.80 mmol, 36.6% yield) as yellow oil. ¹H NMR (MeOD, 400 MHz) δ8.01 (t, J=1.6 Hz, 1H), 7.96-7.91 (m, 1H), 7.86 (d, J=8.0 Hz, 1H), 7.66-7.60 (m, 1H), 3.98-3.90 (m, 2H), 3.47 (dd, J=6.0, 8.4 Hz, 2H), 2.74-2.60 (m, 1H), 2.28 (d, J=7.6 Hz, 2H), 2.15 (s, 6H). LC/MS [M+H]333.0 (calculated); LC/MS [M+H]333.0 (observed).

Preparation of BzL-42d: To a mixture of BzL-42c (299 mg, 898 umol, 1.1 eq) and tert-butyl N-[3-[[2-amino-8-(4,4,5,5-tetramethyl-1,3,2-dioxaborolan-2-yl)-3H-1-benzazepine-4-carbonyl]-propyl-amino]propyl]carbamate (0.43 g, 817 umol, 1.0 eq) in dioxane (10 mL), H₂O (1 mL) was added K₂CO₃ (395 mg, 2.86 mmol, 3.5 eq), Pd(dppf)C₁₂ (29.9 mg, 40.8 umol, 0.05 eq) at 25° C. under N₂ and then stirred at 100° C. for 2 h. The mixture was filtered, diluted with water and extracted with EtOAc (30 mL×3). The organic layer was washed with brine, dried over Na₂SO₄, filtered and concentrated. The residue was purified by silica gel chromatography (column height: 250 mm, diameter: 100 mm, 100-200 mesh silica gel, Petroleum ether/Ethyl acetate=1/0, 0/1) to afford tert-butylN-[3-[[2-amino-8-[3-[3-[(dimethylamino)methyl]azetidin-1-yl]sulfonylphenyl]-3H-1-benzazepine-4-carbonyl]-propyl-amino]propyl]carbamate, BzL-42d (0.3 g, 459 umol, 56.3% yield) as yellow solid.

Preparation of BzL-42e: To a mixture of BzL-42d (0.25 g, 383 umol, 1.0 eq) in DCM (2 mL) was added TFA (1.31 g, 11.5 mmol, 851 μL, 30.0 eq) in one portion at 25° C. and then stirred for 1 h. The mixture was concentrated to afford 2-amino-N-(3-aminopropyl)-8-[3-[3-[(dimethylamino)methyl]azetidin-1-yl]sulfonylphenyl]-N-propyl-3H-1-benzazepine-4-carboxamide, BzL-42e (0.2 g, crude) as a yellow oil.

Preparation of BzL-42f: To a mixture of BzL-42e (0.2 g, 362 umol, 1.0 eq) in DMF (0.5 mL) was added Et₃N (256 mg, 2.53 mmol, 353 μL, 7.0 eq) and 3-isothiocyanatobenzonitrile (52.2 mg, 326 umol, 0.9 eq) at 25° C. and then stirred at this temperature for 1 h. The mixture was filtered and the filtrate was purified by prep-HPLC(column: Welch Xtimate C18 100*25 mm*3 um; mobile phase: [water(0.1% TFA)-ACN]; B %: 10%-40%, 12 min) to give 2-amino-N-[3-[(3-cyanophenyl) carbamothioylamino]propyl]-8-[3-[3-[(dimethylamino)methyl]azetidin-1-yl]sulfonylphenyl]-N-propyl-3H-1-benzazepine-4-carboxamide, BzL-42f (0.18 g, 252 umol, 69.8% yield) as yellow solid. ¹H NMR (MeOD, 400 MHz) δ8.12-8.06 (m, 2H), 7.92-7.02 (m, 10H), 4.01 (t, J=8.4 Hz, 2H), 3.76-3.40 (m, 8H), 3.40-3.36 (m, 2H), 3.34-3.32 (m, 2H), 3.03-2.91 (m, 1H), 2.82 (s, 6H), 2.04 (s, 2H), 1.77-1.67 (m, 2H), 0.97 (s, 3H).

Preparation of BzL-42g: To a mixture of BzL-42f (0.14 g, 196 umol, 1.0 eq) and tert-butyl3-[2-[2-[2-[2-[2-[2-[2-[2-[2-(2-aminoethoxy) ethoxy]ethoxy]ethoxy]ethoxy]ethoxy]ethoxy]ethoxy]ethoxy]ethoxy]propanoate (138 mg, 236 umol, 1.2 eq) in DMF (0.5 mL) was added Et₃N (40.0 mg, 393 umol, 2.0 eq) and HgCl₂ (64.0 mg, 236 umol, 1.2 eq) at 25° C. and then stirred for 18 h at this temperature. The mixture was filtered and the filtrate was purified by prep-HPLC(column: Nano-micro Kromasil C18 100*30 mm 8 um; mobile phase: [water (0.1% TFA)-ACN]; B %: 15%-45%, 10 min) to give tert-butyl3-[2-[2-[2-[2-[2-[2-[2-[2-[2-[2-[[(Z)-N-[3-[[2-amino-8-[3-[3-[(dimethylamino)methyl]azetidin-1-yl]sulfonylphenyl]-3H-1-benzazepine-4-carbonyl]-propyl-amino]propyl]-N′-(3-cyanophenyl)carbamimidoyl]amino]ethoxy]ethoxy]ethoxy]ethoxy]ethoxy]ethoxy]ethoxy]ethoxy]ethoxy]ethoxy]propanoate, BzL-42g (0.14 g, 111 umol, 56.4% yield) as yellow oil.

Preparation of BzL-42h: To a solution of BzL-42g (0.12 g, 94.9 umol, 1.0 eq) in H₂O (2 mL) and CH₃CN (0.5 mL) was added TFA (325 mg, 2.85 mmol, 211 μL, 30.0 eq) at 25° C. and then stirred at 80° C. for 1 h. The mixture was concentrated in vacuum to give a residue, the residue was purified by prep-HPLC(column: Xtimate C18 100*30 mm*3 um; mobile phase: [water(0.1% TFA)-ACN]; B %: 5%-35%, 10 min) to give 3-[2-[2-[2-[2-[2-[2-[2-[2-[2-[2-[[(Z)-N-[3-[[2-amino-8-[3-[3-[(dimethylamino)methyl]azetidin-1-yl]sulfonylphenyl]-3H-1-benzazepine-4-carbonyl]-propyl-amino]propyl]-N′-(3-cyanophenyl)carbamimidoyl]amino]ethoxy]ethoxy]ethoxy]ethoxy]ethoxy]ethoxy]ethoxy]ethoxy]ethoxy]ethoxy]propanoic acid, BzL-42h (32 mg, 26.5 umol, 27.9% yield) as yellow oil. ¹H NMR (MeOD, 400 MHz) δ8.16-8.09 (m, 2H), 7.93 (d, J=8.0 Hz, 1H), 7.87-7.81 (m, 1H), 7.81-7.74 (m, 3H), 7.66-7.62 (m, 4H), 7.12 (s, 1H), 4.01 (t, J=8.4 Hz, 2H), 3.80-3.66 (m, 10H), 3.66-3.45 (m, 40H), 3.40 (s, 3H), 2.82 (s, 6H), 2.53 (t, J=6.4 Hz, 2H), 2.07-2.01 (m, 1H), 1.77-1.67 (m, 2H), 0.98-0.90 (m, 3H). LC/MS [M+H]1208.6 (calculated); LC/MS [M+H]1208.6 (observed).

Preparation of BzL-42: BzL-42h (0.032 g, 0.026 mmol, 1 eq.) and TFP (0.009 g, 0.05 mmol, 2 eq.) were dissolved in DMF. Collidine (0.017 ml, 0.13 mmol, 5 eq.) was added, followed by EDC-HCl (0.015 g, 0.079 mmol, 3 eq.). The reaction was stirred at room temperature and monitored by LCMS, then concentrated and purified by HPLC to give 2,3,5,6-tetrafluorophenyl 40-(2-amino-8-(3-((3-((dimethylamino)methyl)azetidin-1-yl)sulfonyl)phenyl)-3H-benzo[b]azepine-4-carbonyl)-35-((3-cyanophenyl)imino)-4,7,10,13,16,19,22,25,28,31-decaoxa-34,36,40-triazatritetracontanoate (0.018 g, 0.013 mmol, 49%). LC/MS [M+H]1356.62 (calculated); LC/MS [M+H]1357.10 (observed).

### Example 63 Synthesis of BzL-43

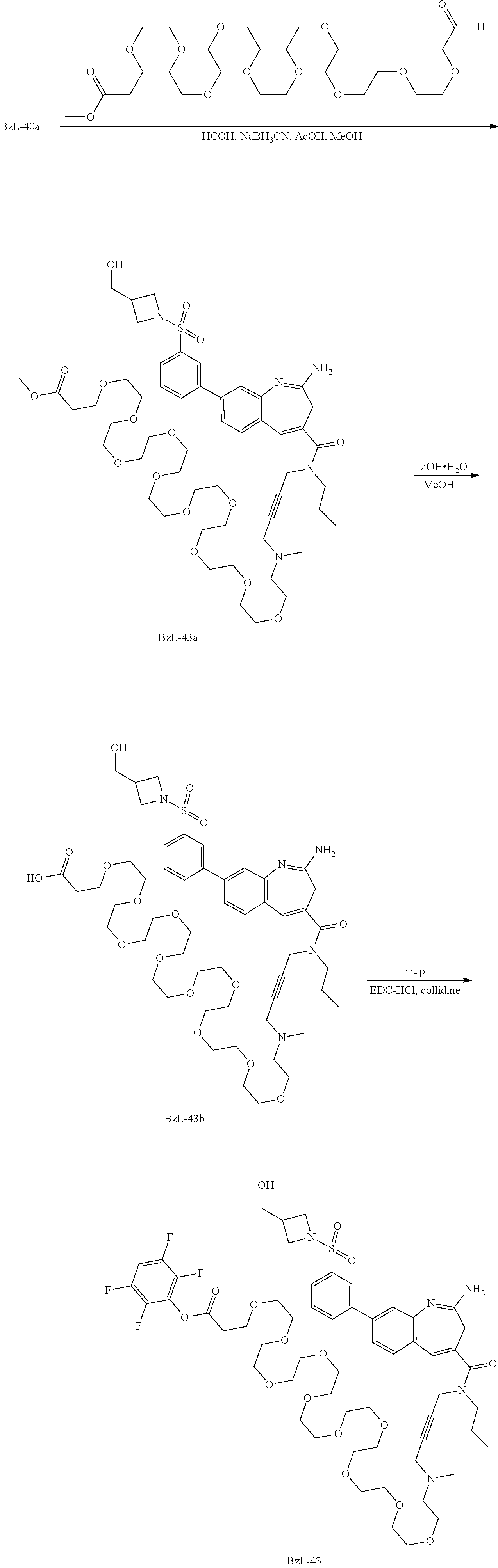

Preparation of BzL-43a: To a mixture of 2-amino-N-(4-aminobut-2-ynyl)-8-[3-[3-(hydroxymethyl)azetidin-1-yl]sulfonylphenyl]-N-propyl-3H-1-benzazepine-4-carboxamide, BzL-40a (0.1 g, 187 umol, 1.0 eq) and methyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (101.3 mg, 187 umol, 1.0 eq) in MeOH (10 mL) was added AcOH (11.2 mg, 187 umol, 11 μL, 1.0 eq) and NaBH₃CN (35.2 mg, 560 umol, 3.0 eq) in one portion at 25° C. and then stirred for 2 hours. Then formaldehyde (29.5 mg, 373 umol, 27 μL, 2.0 eq) was added and it was stirred for 1 hour at the same temperature. The mixture was added a few drops water and concentrated. The residue was purified by prep-HPLC (column: Xtimate C18 100*30 mm*3 um; mobile phase: [water(0.1% TFA)-ACN]; B %: 10%-35%, 10 min) to give methyl 3-[2-[2-[2-[2-[2-[2-[2-[2-[2-[2-[4-[[2-amino-8-[3-[3-(hydroxymethyl)azetidin-1-yl]sulfonylphenyl]-3H-1-benzazepine-4-carbonyl]-propyl-amino]but-2-ynyl-methyl-amino]ethoxy]ethoxy]ethoxy]ethoxy]ethoxy]ethoxy]ethoxy]ethoxy]ethoxy]ethoxy]propanoate, BzL-43a (0.05 g, 46.46 umol, 24.88% yield) as colorless oil.

Preparation of BzL-43b: To a solution of BzL-43a (50 mg, 46.5 umol, 1.0 eq) in MeOH (3.0 mL) and H₂O (0.3 mL) was added LiOH·H₂O (19.5 mg, 465 umol, 10.0 eq) in one portion at 25° C. and it was stirred at the same temperature for 16 hours. The mixture was cooled to 0° C., adjusted pH=7 with aq HCl(1M) and concentrated in reduced pressure at 40° C. The residue was purified by prep-HPLC (column: Nano-micro Kromasil C18 100*30 mm 8 um; mobile phase: [water (0.1% TFA)-ACN]; B %: 10%-40%, 10 min) to afford 3-[2-[2-[2-[2-[2-[2-[2-[2-[2-[2-[4-[[2-amino-8-[3-[3-(hydroxymethyl)azetidin-1-yl]sulfonylphenyl]-3H-1-benzazepine-4-carbonyl]-propyl-amino]but-2-ynyl-methyl-amino]ethoxy]ethoxy]ethoxy]ethoxy]ethoxy]ethoxy]ethoxy]ethoxy]ethoxy]ethoxy]propanoic acid, BzL-43b (30 mg, 28.24 umol, 60.79% yield) as light yellow oil. ¹H NMR (MeOD, 400 MHz) δ8.15-8.07 (m, 2H), 7.93 (d, J=8.0 Hz, 1H), 7.86-7.76 (m, 3H), 7.74-7.69 (m, 1H), 7.24 (s, 1H), 4.29 (s, 2H), 3.91-3.84 (m, 4H), 3.74-3.55 (m, 43H), 3.52-3.38 (m, 7H), 3.34-3.32 (m, 2H), 3.02 (s, 3H), 2.64-2.56 (m, 1H), 2.53 (t, J=6.4 Hz, 2H), 1.85-1.72 (m, 2H), 0.98 (t, J=7.2 Hz, 3H). LC/MS [M+H]1062.5 (calculated); LC/MS [M+H]1062.6 (observed).

Preparation of BzL-43: Bz-43b (0.03 g, 0.028 mmol, 1 eq.) and TFP (0.009 g, 0.06 mmol, 2 eq.) were dissolved in DMF. Collidine (0.019 ml, 0.14 mmol, 5 eq.) was added, followed by EDC-HCl (0.016 g, 0.085 mmol, 3 eq.). The reaction was stirred at room temperature and monitored by LCMS, then concentrated and purified by HPLC to give 2,3,5,6-tetrafluorophenyl 38-(2-amino-8-(3-((3-((dimethylamino)methyl)azetidin-1-yl)sulfonyl)phenyl)-3H-benzo[b]azepine-4-carbonyl)-33-methyl-3,6,9,12,15,18,21,24,27,30-decaoxa-33,38-diazahentetracont-35-ynoate, BzL-43 (0.016 g, 0.013 mmol, 46%). LC/MS [M+H]1210.53 (calculated); LC/MS [M+H]1210.95 (observed).

### Example 64 Synthesis of BzL-44

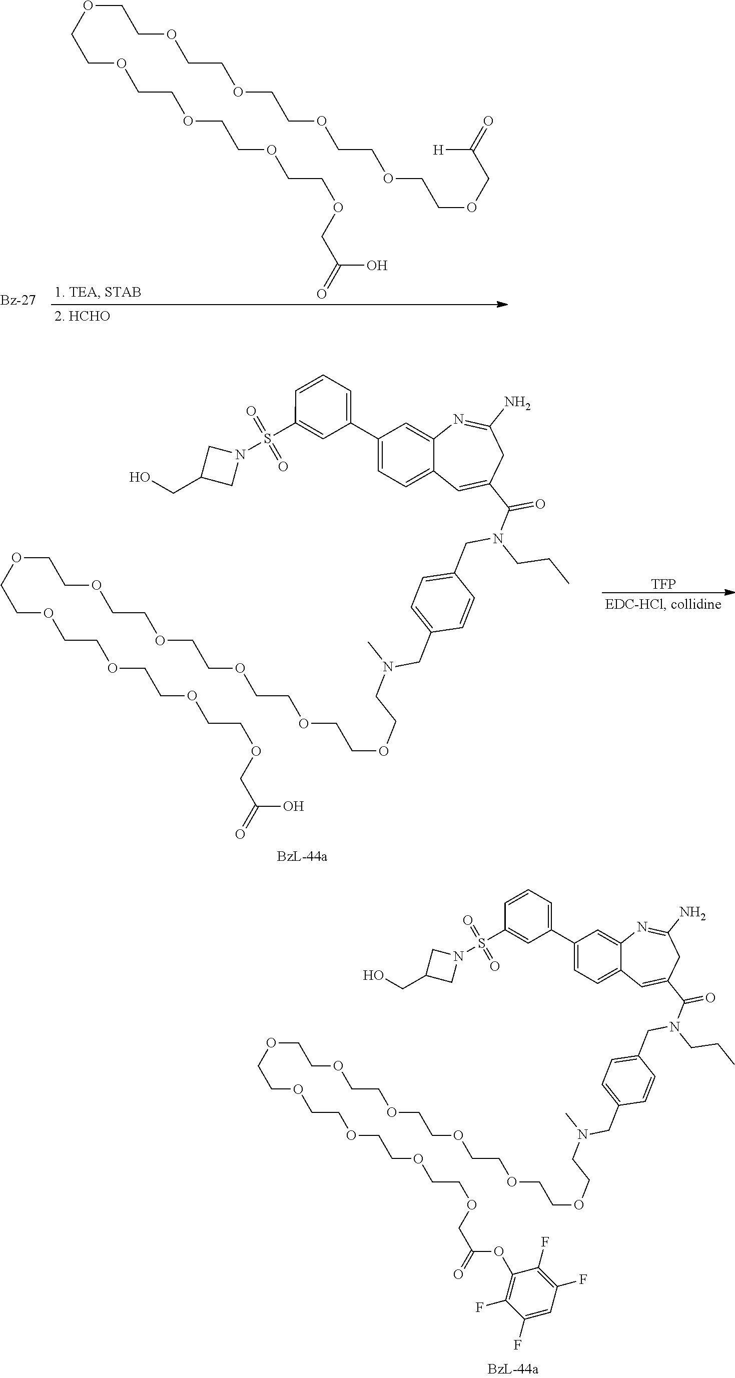 [SMILES: O=CCOCCOCCOCCOCCOCCOCCOCCOCCOCCOCC(=O)O ; CCCN(Cc1ccc(CN(C)CCOCCOCCOCCOCCOCCOCCOCCOCCOCCOCC(=O)O)cc1)C(=O)C1=Cc2ccc(-c3cccc(S(=O)(=O)N4CC(CO)C4)c3)cc2N=C(N)C1 ; "TFP" ; CCCN(Cc1ccc(CN(C)CCOCCOCCOCCOCCOCCOCCOCCOCCOCCOCC(=O)Oc2c(F)c(F)cc(F)c2F)cc1)C(=O)C1=Cc2ccc(-c3cccc(S(=O)(=O)N4CC(CO)C4)c3)cc2N=C(N)C1]

Preparation of BzL44a: 2-Amino-N-(4-(aminomethyl)benzyl)-8-(3-((3-(hydroxymethyl)azetidin-1-yl)sulfonyl)phenyl)-N-propyl-3H-benzo[b]azepine-4-carboxamide, Bz-27 (0.119 g, 0.203 mmol, 1 eq.) and 32-oxo-3,6,9,12,15,18,21,24,27,30-decaoxadotriacontanoic acid (0.107 g, 0.203 mmol, 1 eq.) were dissolved in 1:1 ACN:DCM. Triethylamine (0.17 ml, 1.2 mmol, 6 eq.) was added, followed by sodium triacetoxyborohydride (0.13 g, 0.61 mmol, 3 eq.). The reaction was stirred at room temperature for 40 minutes, and then formaldehyde was added (0.02 ml, 0.27 mmol, 1.3 eq., 37 wt. % in H₂O). After 10 minutes, the reaction was concentrated and purified by HPLC to give 1-(4-((2-amino-8-(3-((3-(hydroxymethyl)azetidin-1-yl)sulfonyl)phenyl)-N-propyl-3H-benzo[b]azepine-4-carboxamido)methyl)phenyl)-2-methyl-5,8,11,14,17,20,23,26,29,32-decaoxa-2-azatetratriacontan-34-oic acid, BzL44a (0.067 g, 0.060 mmol, 30%). LC/MS [M+H]1114.5 (calculated); LC/MS [M+H]1114.89 (observed).

Preparation of BzL-44: BzL-44a (0.067 g, 0.06 mmol, 1 eq.) and TFP (0.020 g, 0.12 mmol, 2 eq.) were dissolved in DMF. Collidine (0.040 ml, 0.30 mmol, 5 eq.) was added, followed by EDC-HCl (0.035 g, 0.18 mmol, 3 eq.). The reaction was stirred at room temperature and monitored by LCMS, then concentrated and purified by HPLC to give 2,3,5,6-tetrafluorophenyl 1-(4-((2-amino-8-(3-((3-(hydroxymethyl)azetidin-1-yl)sulfonyl)phenyl)-N-propyl-3H-benzo[b]azepine-4-carboxamido)methyl)phenyl)-2-methyl-5,8,11,14,17,20,23,26,29,32-decaoxa-2-azatetratriacontan-34-oate, BzL-44 (0.026 g, 0.021 mmol, 34%). LC/MS [M+H]1262.56 (calculated); LC/MS [M+H]1262.86 (observed).

### Example 65 Synthesis of BzL-45

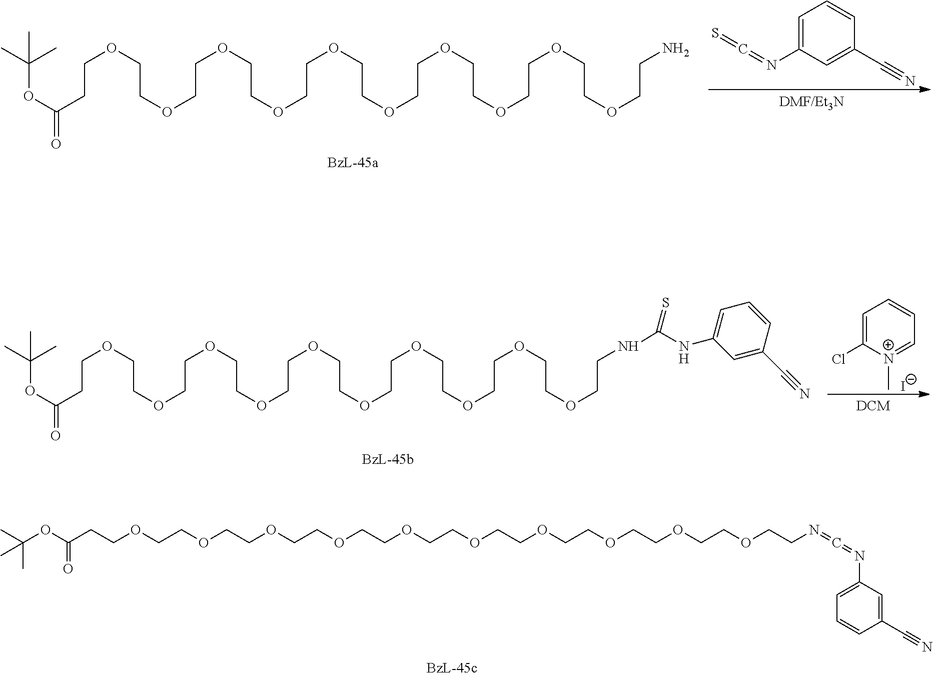

Preparation of BzL-45b: To a mixture of tert-butyl 3-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]propanoate, BzL-45a (2.7 g, 4.61 mmol, 1.0 eq) in THE (20 mL) was added Et₃N (700 mg, 6.91 mmol, 960 μL, 1.5 eq) and 3-isothiocyanatobenzonitrile (1.48 g, 9.22 mmol, 2.0 eq) at 25° C. and it was stirred for 1 hour at this temperature. Then the mixture was diluted with water (30 mL) and extracted with EtOAc (50 mL×3). The organic layer was washed with brine, dried over Na₂SO₄, filtered and concentrated. The residue was purified by silica gel chromatography (MeOH/Ethyl acetate=0/1, 1/10) to afford tert-butyl 3-[2-[2-[2-[2-[2-[2-[2-[2-[2-[2-[(3-cyanophenyl)carbamothioylamino]ethoxy]ethoxy]ethoxy]ethoxy]ethoxy]ethoxy]ethoxy]ethoxy]ethoxy]ethoxy]propanoate, BzL-45b (0.5 g, 670 umol, 14.54% yield) as yellow oil. ¹H NMR (CDCl₃, 400 MHz) δ7.99 (s, 1H), 7.89 (d, J=8.0 Hz, 1H), 7.44-7.39 (m, 2H), 3.76-3.58 (m, 42H), 2.55-2.46 (m, 2H), 1.45 (s, 9H).

Preparation of BzL-45c: To a mixture of BzL-45b (0.4 g, 536 umol, 1.0 eq) and Et₃N (163 mg, 1.61 mmol, 223 μL, 3.0 eq) in DCM (10 mL) and DMF (0.4 mL) was added 2-chloro-1-methylpyridin-1-ium iodide (164 mg, 643 umol, 1.2 eq) at 25° C. under N₂. The mixture was stirred at 25° C. for 1 hour and then concentrated under reduce pressure. The residue was purified by silica gel chromatography (CH₃CN/Ethyl acetate=0/1 to 1/1) to afford tert-butyl 3-[2-[2-[2-[2-[2-[2-[2-[2-[2-[2-[(3-cyanophenyl)iminomethyleneamino]ethoxy]ethoxy]ethoxy]ethoxy]ethoxy]ethoxy]ethoxy]ethox y]ethoxy]ethoxy]propanoate, BzL-45c (0.29 g, 407 umol, 75.9% yield) as yellow oil. ¹H NMR (CDCl₃, 400 MHz) δ7.43-7.33 (m, 4H), 3.70-3.62 (m, 42H), 2.51 (t, J=6.4 Hz, 2H), 1.45 (s, 9H).

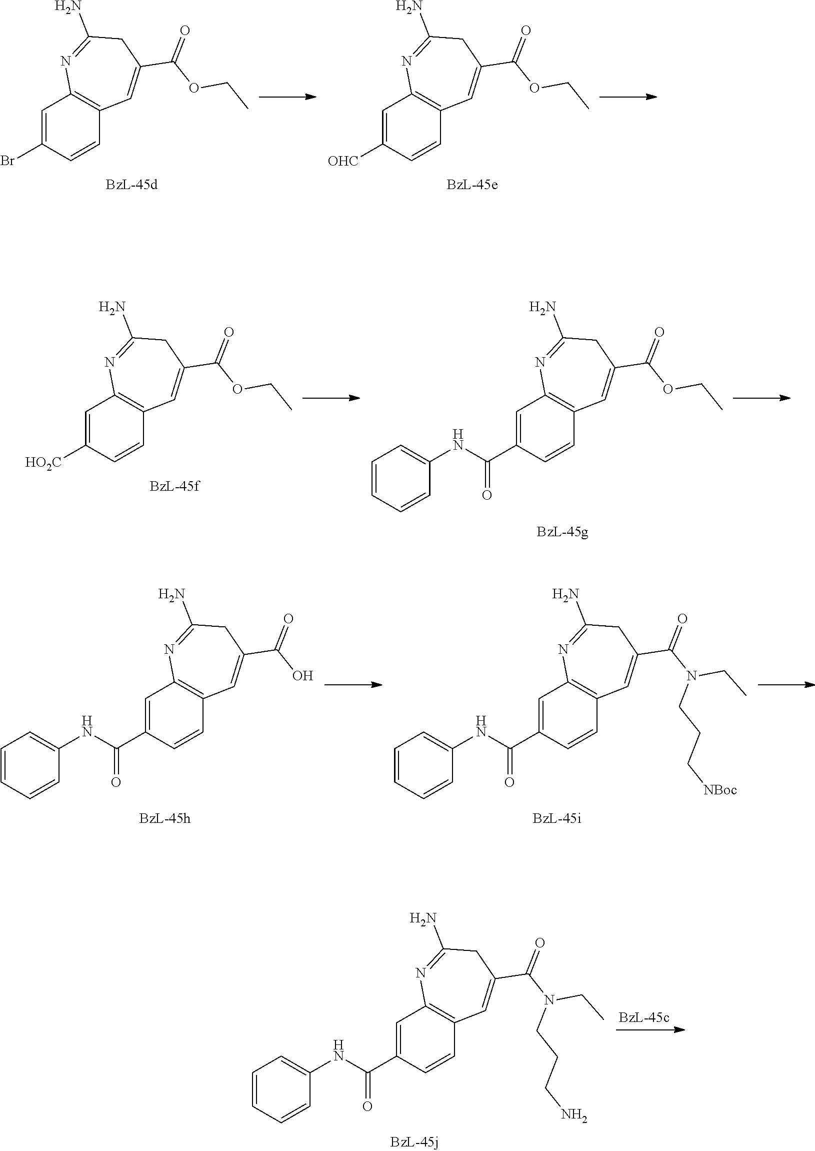

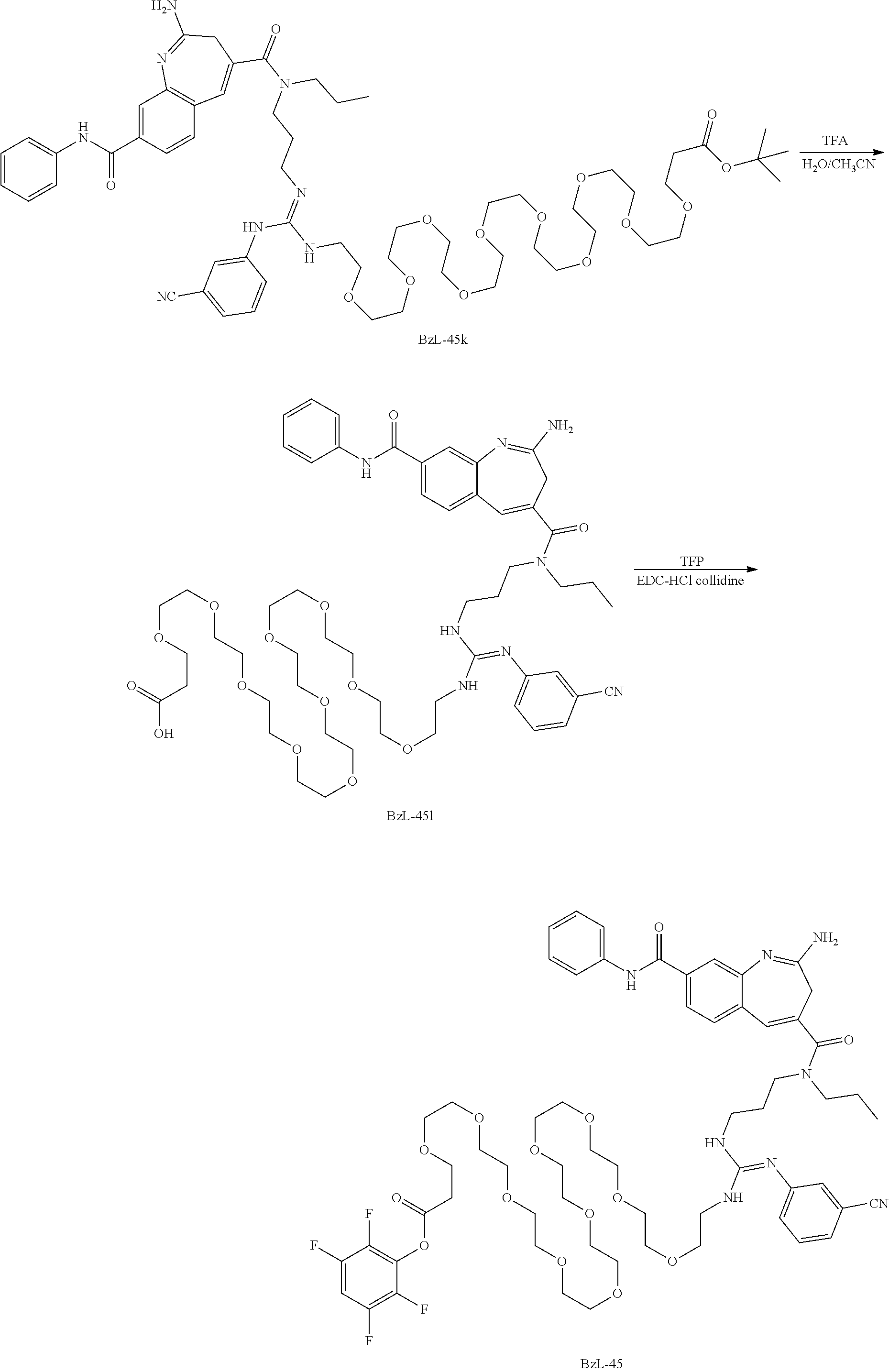 [SMILES: CCCN(CCC/N=C(/NCCOCCOCCOCCOCCOCCOCCOCCOCCOCCOCCC(=O)OC(C)(C)C)Nc1cccc(C#N)c1)C(=O)C1=Cc2ccc(C(=O)Nc3ccccc3)cc2N=C(N)C1 ; CCCN(CCCN/C(=N/c1cccc(C#N)c1)NCCOCCOCCOCCOCCOCCOCCOCCOCCOCCOCCC(=O)O)C(=O)C1=Cc2ccc(C(=O)Nc3ccccc3)cc2N=C(N)C1 ; "TFP" ; CCCN(CCCN/C(=N/c1cccc(C#N)c1)NCCOCCOCCOCCOCCOCCOCCOCCOCCOCCOCCC(=O)Oc1c(F)c(F)cc(F)c1F)C(=O)C1=Cc2ccc(C(=O)Nc3ccccc3)cc2N=C(N)C1]

Preparation of BzL-45e: To a solution of ethyl 2-amino-8-bromo-3H-1-benzazepine-4-carboxylate, BzL-45d (10 g, 32.4 mmol, 1.0 eq) in DMF (100 mL) was added Et₃SiH (72.8 g, 626.09 mmol, 100 mL, 19.36 eq), Et₃N (6.5 g, 64.69 mmol, 9.00 mL, 2.0 eq) and Pd(dppf)C₁₂ (1.18 g, 1.62 mmol, 0.05 eq) under N₂. The suspension was degassed under vacuum and purged with CO several times and it was stirred under CO (50 psi) at 80° C. for 12 h. The mixture was diluted with water (300 mL) and extracted with EtOAc (80 mL×3). The organic layer was washed with brine (50 mL), dried over Na₂SO₄, filtered and concentrated, and the residue was purified by flash silica gel chromatography (ISCO®; 15 g SepaFlash® Silica Flash Column, Eluent of 0-100% Ethyl acetate/Petroleum ether gradient @65 mL/min) to give ethyl 2-amino-8-formyl-3H-1-benzazepine-4-carboxylate, BzL-45e (3 g, 11.6 mmol, 35.9% yield) as yellow solid. ¹H NMR (DMSO-d₆, 400 MHz) δ10.00 (s, 1H) 7.79 (s, 1H) 7.61 (d, J=8.4 Hz, 1H) 7.55 (d, J=1.2 Hz, 1H) 7.40 (dd, J=8.0, 1.2 Hz, 1H) 7.07 (s, 2H) 4.25 (q, J=6.8 Hz, 2H) 2.91 (s, 2H) 1.31 (t, J=6.8 Hz, 3H).

Preparation of BzL-45f: To a solution of BzL-45e (2.6 g, 10.1 mmol, 1.0 eq) in CH₃CN (15 mL) was added NaH₂PO₄ (362 mg, 3.02 mmol, 0.3 eq), H₂O₂(5.71 g, 50.33 mmol, 4.84 mL, 30% purity, 5.0 eq) and NaClO₂ (1.46 g, 16.1 mmol, 1.6 eq) at 0° C. and it was stirred at 25° C. for 5 h. The reaction mixture was quenched with Na₂SO₃ (aq) and diluted with H₂O (30 mL) and EtOAc (30 ml), the pH of the mixture was adjusted to 4 with aq HCl (1 M), then filtered to give desired solid The solid was dried under vacuum to give 2-amino-4-ethoxycarbonyl-3H-1-benzazepine-8-carboxylic acid, BzL-45f (2.1 g, 7.66 mmol, 76.1% yield) as white solid. ¹H NMR (DMSO-d₆, 400 MHz) δ7.87 (s, 1H), 7.81 (s, 1H), 7.72-7.67 (m, 2H), 4.27 (q, J=7.2 Hz, 2H), 3.28 (s, 2H), 1.31 (t, J=7.2 Hz, 3H).

Preparation of BzL-45g: To a mixture of BzL-45f (1.0 g, 3.65 mmol, 1.0 eq) in DMF 25 (20 mL) was added PYAOP (2.28 g, 4.38 mmol, 1.2 eq) and DIEA (2.36 g, 18.2 mmol, 3.18 mL, 5.0 eq) at 25° C. and it was stirred for 10 min, then aniline (373 mg, 4.01 mmol, 366 μL, 1.1 eq) was added and stirred for 1 hour at 25° C. The mixture was poured into ice water (50 mL) and stirred for 2 min. The aqueous phase was extracted with ethyl acetate (20 mL×3). The combined organic phase was washed with brine (20 mL), dried with anhydrous Na₂SO₄, filtered and concentrated in vacuum and the residue was purified by silica gel chromatography (EtOAc/MeOH=1:0˜2:1) to afford ethyl 2-amino-8-(phenylcarbamoyl)-3H-1-benzazepine-4-carboxylate, BzL-45g (0.5 g, 1.43 mmol, 39.25% yield) as yellow solid. ¹H NMR (MeOD, 400 MHz) δ 7.89 (s, 1H), 7.76-7.65 (m, 3H), 7.62-7.56 (m, 1H), 7.37 (t, J=8.0 Hz, 2H), 7.16 (t, J=8.0 Hz, 1H), 4.35 (q, J=7.2 Hz, 2H), 3.32 (s, 2H), 1.38 (t, J=7.2 Hz, 3H).

Preparation of BzL-45h: To a mixture of BzL-45g (0.36 g, 1.03 mmol, 1.0 eq) in EtOH (10 mL) was added a solution of LiOH·H₂O (216 mg, 5.15 mmol, 5.0 eq) in H₂O (1 mL) at 25° C. and it was stirred for 16 hours at this temperature. The mixture was quenched with HCl (4M) until pH to 5 and concentrated under reduced pressure at 40° C. to remove EtOH. Water (10 mL) was added and then filtered to give 2-amino-8-(phenylcarbamoyl)-3H-1-benzazepine-4-carboxylic acid, BzL-45h (0.2 g, 622 umol, 60.41% yield) as yellow solid which was used in the next step without further purification. ¹H NMR (DMSO-d₆, 400 MHz) δ7.84-7.74 (m, 3H), 7.66 (s, 1H), 7.56-7.47 (m, 2H), 7.34 (t, J=8.0 Hz, 2H), 7.09 (t, J=7.2 Hz, 2H), 2.92 (s, 2H).

Preparation of BzL-45i: To a solution of BzL-45h (0.2 g, 622 umol, 1.0 eq) in DMF (5 mL) was added HATU (284 mg, 746 umol, 1.2 eq) and DIEA (241 mg, 1.87 mmol, 325 μL, 3.0 eq) at 25° C. and it was stirred for 10 min at this temperature, then tert-butyl N-[3-(propylamino)propyl]carbamate, Bz-1b (161 mg, 746 umol, 1.2 eq) was added to the mixture and stirred at 25° C. for 3 hours. The reaction was poured into ice water (30 mL) and stirred for 10 min. The aqueous phase was extracted with EtOAc (10 mL×3), and the combined organic phase was washed with H₂O (10 mL×2) and brine (10 mL), dried by Na₂SO₄ and concentrated to give tert-butyl N-[3-[[2-amino-8-(phenylcarbamoyl)-3H-1-benzazepine-4-carbonyl]-propyl-amino]propyl]carbamate, BzL-45i (0.3 g, 577 umol, 92.76% yield) as yellow oil.

Preparation of BzL-45j: To a solution of BzL-45i (0.4 g, 769 umol, 1.0 eq) in MeOH (10 mL) was added HCl/MeOH (4 M, 9.62 mL, 50 eq) at 25° C. The mixture was stirred at 25° C. for 1 hour, and then concentrated under reduced pressure at 40° C. The residue was purified by prep-HPLC (column: Nano-micro Kromasil C18 100*30 mm 8 um; mobile phase: [water (0.1% TFA) -ACN]; B %: 5%-30%, 10 min) to afford 2-amino-N4-(3-aminopropyl)-N8-phenyl-N4-propyl-3H-1-benzazepine-4,8-dicarboxamide, BzL-45j (0.23 g, 431 umol, 56.0% yield, TFA salt) as yellow solid. ¹H NMR (MeOD, 400 MHz) δ8.01-7.94 (m, 2H), 7.76-7.70 (m, 3H), 7.41 (t, J=8.0 Hz, 2H), 7.21 (t, J=7.6 Hz, 2H), 3.63 (t, J=7.2 Hz, 2H), 3.58-3.49 (m, 2H), 3.41 (s, 2H), 3.10-2.95 (m, 2H), 2.12-1.99 (m, 2H), 1.82-1.68 (m, 2H), 0.95 (t, J=7.2 Hz, 3H). LC/MS [M+H]420.2 (calculated); LC/MS [M+H]420.2 (observed).

Preparation of BzL-45k: To a mixture of Bz-45j (0.06 g, 112 umol, 1.0 eq, TFA salt) in DMF (1 mL) was added Et₃N (28 mg, 281 umol, 2.5 eq) and BzL-45c (88 mg, 123 umol, 1.1 eq) at 25° C. The mixture was stirred at 25° C. for 1 hour and then filtered and purified by prep-HPLC (column: Nano-micro Kromasil C18 100*30 mm 8 um; mobile phase: [water (0.1% TFA)-ACN]; B %: 20%-50%, 10 min) to afford tert-butyl 3-[2-[2-[2-[2-[2-[2-[2-[2-[2-[2-[[(Z)-N′-[3-[[2-amino-8-(phenylcarbamoyl)-3H-1-benzazepine-4-carbonyl]-propyl-amino]propyl]-N-(3-cyanophenyl)carbamimidoyl]amino]ethoxy]ethoxy]ethoxy]ethoxy]ethoxy]ethoxy]ethoxy]ethoxy]ethoxy]ethoxy]propanoate, BzL-45k (0.08 g, 70.7 umol, 62.9% yield) as colorless oil.

Preparation of BzL-451: To a solution of BzL-45k (0.07 g, 61 umol, 1.0 eq) in H₂O (5 mL) and CH₃CN (1 mL) was added TFA (211 mg, 1.86 mmol, 30 eq) at 25° C. The mixture was stirred at 80° C. for 2 hours and then concentrated under reduced pressure. The residue was freeze-dried to give 3-[2-[2-[2-[2-[2-[2-[2-[2-[2-[2-[[(Z)-N′-[3-[[2-amino-8-(phenylcarbamoyl)-3H-1-benzazepine-4-carbonyl]-propyl-amino]propyl]-N-(3-cyanophenyl)carbamimidoyl]amino]ethoxy]ethoxy]ethoxy]ethoxy]ethoxy]ethoxy]ethoxy]ethoxy]ethoxy]ethoxy]propanoic acid, BzL-45l (51 mg, 42.9 umol, 69.3% yield, TFA salt) as light yellow oil. ¹H NMR (MeOD, 400 MHz) δ8.01-7.94 (m, 2H), 7.79-7.75 (m, 1H), 7.72 (d, J=8.0 Hz, 2H), 7.66-7.64 (m, 4H), 7.39 (t, J=7.6 Hz, 2H), 7.19 (t, J=7.6 Hz, 1H), 7.13 (s, 1H), 3.76-3.52 (m, 46H), 3.42-3.40 (m, 4H), 2.53 (t, J=6.4 Hz, 2H), 2.04 (m, 2H), 1.79-1.65 (m, 2H), 0.93 (t, J=7.2 Hz, 3H). LC/MS [M+H]1075.6 (calculated); LC/MS [M+H]1075.6 (observed).

Preparation of BzL-45: BzL-45l (0.051 g, 0.047 mmol, 1 eq.) and TFP (0.016 g, 0.095 mmol, 2 eq.) were dissolved in DMF. Collidine (0.031 ml, 0.24 mmol, 5 eq.) was added, followed by EDC-HCl (0.027 g, 0.14 mmol, 3 eq.). The reaction was stirred at room temperature and monitored by LCMS, then concentrated and purified by HPLC to give 2,3,5,6-tetrafluorophenyl 40-(2-amino-8-(phenylcarbamoyl)-3H-benzo[b]azepine-4-carbonyl)-35-((3-cyanophenyl)imino)-4,7,10,13,16,19,22,25,28,31-decaoxa-34,36,40-triazatritetracontanoate, BzL-45 (0.043 g, 0.035 mmol, 74%). LC/MS [M+H]1223.56 (calculated); LC/MS [M+H]1223.87 (observed).

### Example 66 Synthesis of BzL-46

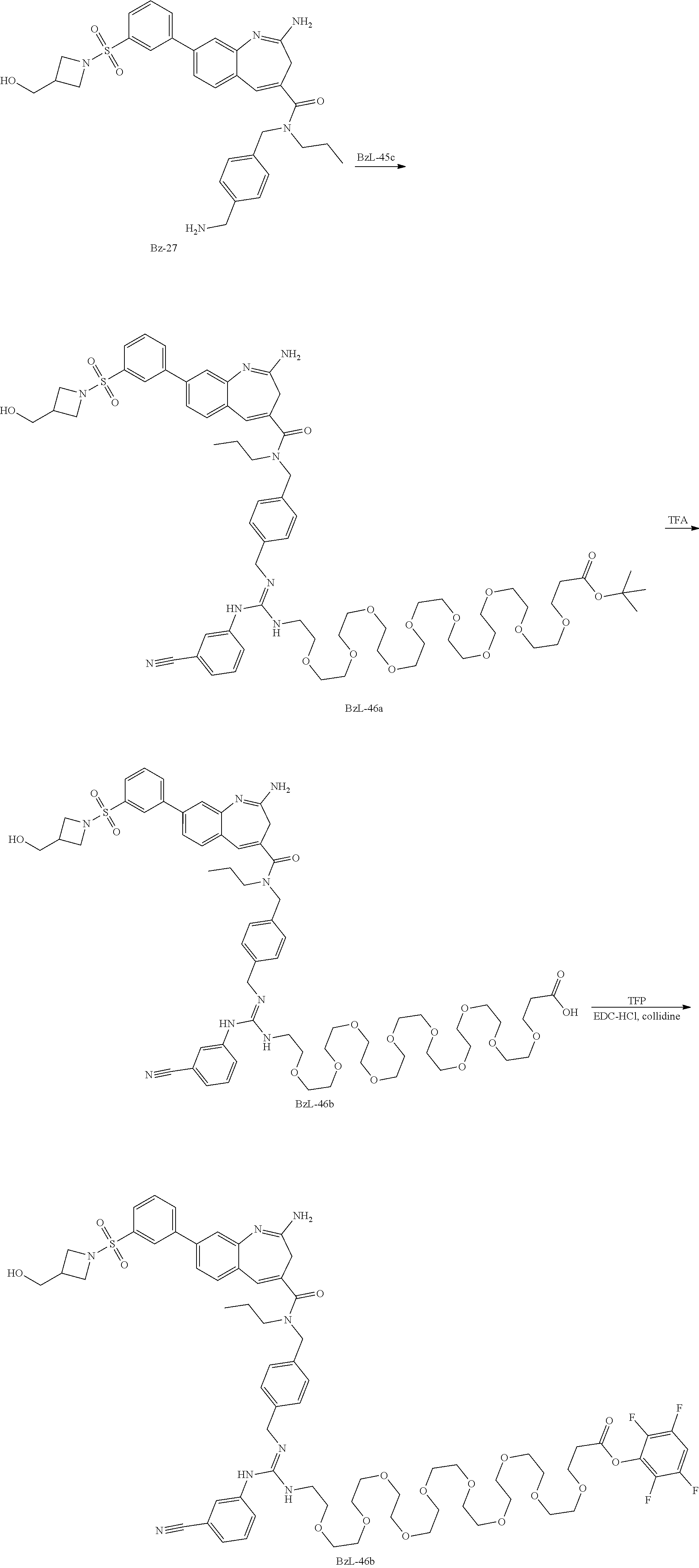

Preparation of BzL-46a: Reaction of Bz-27 and BzL-45c gave tert-butyl (Z)-1-(4-((2-amino-8-(3-((3-(hydroxymethyl)azetidin-1-yl)sulfonyl)phenyl)-N-propyl-3H-benzo[b]azepine-4-carboxamido)methyl)phenyl)-3-((3-cyanophenyl)amino)-7,10,13,16,19,22,25,28,31,34-decaoxa-2,4-diazaheptatriacont-2-en-37-oate, BzL-46a by the procedures described for BzL-42. LC/MS [M+H]1299.7 (calculated); LC/MS [M+H]1299.7 (observed).

Preparation of BzL-46b: Reaction of BzL-46a with trifluoroacetic acid, TFA by the procedures described in the synthesis of BzL-42 gave (Z)-1-(4-((2-amino-8-(3-((3-(hydroxymethyl)azetidin-1-yl)sulfonyl)phenyl)-N-propyl-3H-benzo[b]azepine-4-carboxamido)methyl)phenyl)-3-((3-cyanophenyl)amino)-7,10,13,16,19,22,25,28,31,34-decaoxa-2,4-diazaheptatriacont-2-en-37-oic acid, BzL-46b. LC/MS [M+H]1243.6 (calculated); LC/MS [M+H]1243.6 (observed).

Preparation of BzL-46: Reaction of BzL-46b with 2,3,5,6-tetrafluorophenol, TFP and EDC-HCl, as described in the procedures for the synthesis of BzL-42 gave 2,3,5,6-tetrafluorophenyl (Z)-1-(4-((2-amino-8-(3-((3-(hydroxymethyl)azetidin-1-yl)sulfonyl)phenyl)-N-propyl-3H-benzo[b]azepine-4-carboxamido)methyl)phenyl)-3-((3-cyanophenyl)amino)-7,10,13,16,19,22,25,28,31,34-decaoxa-2,4-diazaheptatriacont-2-en-37-oate, BzL-46. LC/MS [M+H]1391.6 (calculated); LC/MS [M+H]1391.6 (observed).

### Example 67 Preparation of Immunoconjugates (IC)

In an exemplary procedure, an antibody is buffer exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, MO). The eluates are then each adjusted to 6 mg/ml using the buffer and then sterile filtered. The antibody at 6 mg/ml is pre-warmed to 30° C. and rapidly mixed with 2-20 (e.g., 7-10) molar equivalents of aminobenzazepine-linker compound of Formula II. The reaction is allowed to proceed for 16 hours at 30° C. and immunioconjugate A is separated from reactants by running over two successive G-25 desalting columns equilibrated in phosphate buffered saline (PBS) at pH 7.2 to provide the Immunoconjugate (IC) of Tables 3a and 3b. Adjuvant-antibody ratio (DAR) is determined by liquid chromatography mass spectrometry analysis using a C4 reverse phase column on an ACQUITY™ UPLC H-class (Waters Corporation, Milford, Massachusetts) connected to a XEVO™ G2-XS TOE mass spectrometer (Waters Corporation).

For conjugation, the antibody may dissolved in a physiological buffer system known in the art that will not adversely impact the stability or antigen-binding specificity of the antibody. Phosphate buffered saline may be used. The aminobenzazepine-linker intermediate compound is dissolved in a solvent system comprising at least one polar aprotic solvent as described elsewhere herein. In some such aspects, aminobenzazepine-linker intermediate is dissolved to a concentration of about 5 mM, 10 mM, about 20 mM, about 30 mM, about 40 mM or about 50 mM, and ranges thereof such as from about 50 mM to about 50 mM or from about 10 mM to about 30 mM in pH 8 Tris buffer (e.g., 50 mM Tris). In some aspects, the aminobenzazepine-linker intermediate is dissolved in DMSO or acetonitrile, or in DMSO. In the conjugation reaction, an equivalent excess of aminobenzazepine-linker intermediate solution is diluted and combined with chilled antibody solution (e.g. from about 1° C. to about 10° C.). The aminobenzazepine-linker intermediate solution may suitably be diluted with at least one polar aprotic solvent and at least one polar protic solvent, examples of which include water, methanol, ethanol, n-propanol, and acetic acid. In some particular aspects the aminobenzazepine-linker intermediate is dissolved in DMSO and diluted with acetonitrile and water prior to admixture with the antibody solution. The molar equivalents of aminobenzazepine-linker intermediate to antibody may be about 1.5:1, about 3:1, about 5:1, about 10:1 about 15:1 or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS, and the reaction is typically complete in from about 1 hour to about 24 hours. After the reaction is complete, a reagent may be added to the reaction mixture to quench the reaction and/or cap unreacted antibody thiol groups. An example of a suitable capping reagent is ethylmaleimide.

Following conjugation according to Example 5, the immunoconjugates may be purified and separated from unconjugated reactants and/or conjugate aggregates by purification methods known in the art such as, for example and not limited to, size exclusion chromatography, hydrophobic interaction chromatography, ion exchange chromatography, chromatofocusing, ultrafiltration, centrifugal ultrafiltration, and combinations thereof. For instance, purification may be preceded by diluting the immunoconjugate, such in 20 mM sodium succinate, pH 5. The diluted solution is applied to a cation exchange column followed by washing with, e.g., at least 10 column volumes of 20 mM sodium succinate, pH 5. The conjugate may be suitably eluted with a buffer such as PBS.

### Example 68 HEK Reporter Assay

HEK293 reporter cells expressing human TLR7 or human TLR8 were purchased from Invivogen and vendor protocols were followed for cellular propagation and experimentation. Briefly, cells were grown to 80-85% confluence at 5% CO₂ in DMEM supplemented with 10% FBS, Zeocin, and Blasticidin. Cells were then seeded in 96-well flat plates at 4×10⁴ cells/well with substrate containing HEK detection medium and immunostimulatory molecules. Activity was measured using a plate reader at 620-655 nm wavelength.

### Example 69 Assessment of Immunoconjugate Activity In Vitro

This example shows that Immunoconjugates of the invention are effective at eliciting myeloid activation, and therefore are useful for the treatment of cancer.

Isolation of Human Antigen Presenting Cells: Human myeloid antigen presenting cells (APCs) were negatively selected from human peripheral blood obtained from healthy blood donors (Stanford Blood Center, Palo Alto, California) by density gradient centrifugation using a ROSETTESEP™ Human Monocyte Enrichment Cocktail (Stem Cell Technologies, Vancouver, Canada) containing monoclonal antibodies against CD14, CD16, CD40, CD86, CD123, and HLA-DR. Immature APCs were subsequently purified to >90% purity via negative selection using an EASYSEP™ Human Monocyte Enrichment Kit (Stem Cell Technologies) without CD16 depletion containing monoclonal antibodies against CD14, CD16, CD40, CD86, CD123, and HLA-DR.

Myeloid APC Activation Assay: 2×10⁵ APCs were incubated in 96-well plates (Corning, Corning, NY) containing iscove's modified dulbecco's medium, EVIDM (Lonza) supplemented with 10% FBS, 100 U/mL penicillin, 100 μg/mL (micrograms per milliliter) streptomycin, 2 mM L-glutamine, sodium pyruvate, non-essential amino acids, and where indicated, various concentrations of unconjugated (naked) PD-L1 or HER2 antibodies and Immunoconjugate P of the invention (as prepared according to the example above). Trastuzumab and avelumab were used as the antibody constructs. Cell-free supernatants were analyzed after 18 hours by ELISA for TNFα secretion.

Activation of myeloid cell types can be measured using various screen assays in which different myeloid populations are utilized. These may include the following: monocytes isolated from healthy donor blood, M-CSF differentiated Macrophages, GM-CSF differentiated Macrophages, GM-CSF+IL-4 monocyte-derived Dendritic Cells, classical Dendritic Cells isolated from healthy donor blood, and myeloid cells polarized to an immunosuppressive state (also referred to as myeloid derived suppressor cells or MDSCs). Examples of MDSC polarized cells include monocytes differentiated toward immunosuppressive state such as M2a M((IL4/IL13), M2c M((IL10/TGFb), GM-CSF/IL6 MDSCs and tumor-educated monocytes (TEM). TEM differentiation can be performed using tumor-conditioned media (e.g. 786.0, MDA-MB-231, HCC1954). Primary tumor-associated myeloid cells may also include primary cells present in dissociated tumor cell suspensions (Discovery Life Sciences).

Assessment of activation of the described populations of myeloid cells may be performed as a mono-culture or as a co-culture with cells expressing the antigen of interest which the ISAC may bind to via the CDR region of the antibody. Following incubation for 18-48 hours, activation may be assessed by upregulation of cell surface co-stimulatory molecules using flow cytometry or by measurement of secreted proinflammatory cytokines. For cytokine measurement, cell-free supernatant is harvested and analyzed by cytokine bead array (e.g. LegendPlex from Biolegend) using flow cytometry.

All references, including publications, patent applications, and patents, cited herein are hereby incorporated by reference to the same extent as if each reference were individually and specifically indicated to be incorporated by reference and were set forth in its entirety herein.

1. An immunoconjugate comprising an antibody covalently attached to one or more aminobenzazepine moieties by a linker, and having Formula I:
Ab-[L-Bza]ₚ I
or a pharmaceutically acceptable salt thereof,
wherein:
Ab is the antibody;
p is an integer from 1 to 8;
Bza is the aminobenzazepine moiety having the formula: 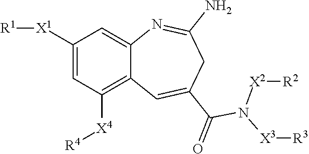
R¹, R², and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-NR⁵—C(═NR^{5a})NR⁵—*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—C(═O)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₅ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)—(C₂-C₅ heteroaryl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
R³ is selected from C₁-C₁₂ alkyl, C₂-C₆ alkenyl, C₂-C₆ alkynyl, —(C₁-C₁₂ alkyldiyl)-N(R⁵)—*; and —(C₁-C₁₂ alkyldiyl)-N(R⁵)₂;
X¹, X², and X⁴ are independently selected from the group consisting of a bond, C(═O), C(═O)N(R⁵), 0, N(R⁵), S, S(O)₂, and S(O)₂N(R⁵);
X³ is O;
R⁵ is selected from the group consisting of H, C₆-C₂₀ aryl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
—C(═O)-(PEG)-;
—C(═O)-(PEG)-C(═O)—;
—C(═O)-(PEG)-O—;
—C(═O)-(PEG)-C(═O)N(R⁵)—(C₁-C₁₂ alkyldiyl)-;
—C(═O)-(PEG)-C(═O)N(R⁵)—(C₁-C₁₂ alkyldiyl)-N(R⁵)C(═O)—(C₂-C₅ monoheterocyclyldiyl)-; and
—C(═O)-(PEG)-N(R⁵)—;
PEG has the formula: —(CH₂CH₂O)ₙ—(CH₂)ₘ—; m is an integer from 1 to 5, and n is an integer from 5 to 20; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)˜H, —OP(O)(OH)₂, —S(O)₂N(CH₃)₂, —SCH₃, —S(O)₂CH₃, and —S(O)₃H.

2. The immunoconjugate of claim 1 wherein X¹ is a bond, and R¹ is C₁-C₂₀ heteroaryl.

3. The immunoconjugate of claim 2 wherein R¹ is pyrimidinyl.

4. The immunoconjugate of claim 3 wherein R¹ is pyrimidinyl substituted with —(C₁-C₁₂ alkyldiyl)-N(R⁵)—*.

5. The immunoconjugate of claim 4 wherein —(C₁-C₁₂ alkyldiyl)-N(R⁵)—* is —CH₂—NH—*.

6. The immunoconjugate of claim 5 wherein —CH₂—NH—* is linked at the asterisk to L, wherein L is —C(═O)-(PEG)-C(═O)—.

7. The immunoconjugate of claim 6 wherein PEG has the formula:
—(CH₂CH₂O)ₙ—(CH₂)ₘ—; where m is 2 and n is 10.

8. The immunoconjugate of claim 1 wherein X² is a bond and R² is C₁-C₁₂ alkyl.

9. The immunoconjugate of claim 1 wherein R³ is C₁-C₁₂ alkyl.

10. The immunoconjugate of claim 1 wherein X²—R² is —CH₂CH₂CH₃, and X³—R³ is —OCH₂CH₃.

11. The immunoconjugate of claim 1 wherein X⁴ is a bond and R⁴ is H.

12. An aminobenzazepine-linker compound of Formula II: 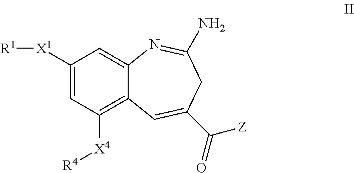
wherein
Z is selected from H, —O(C₁-C₅ alkyl), and N(X²—R²)(X³—R³);
R¹, R², and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-NR⁵—C(═NR^{5a})NR⁵—*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—C(═O)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₅ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)—(C₂-C₅ heteroaryl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
R³ is selected from C₁-C₁₂ alkyl, C₂-C₆ alkenyl, C₂-C₆ alkynyl, —(C₁-C₁₂ alkyldiyl)-N(R⁵)—*; and —(C₁-C₁₂ alkyldiyl)-N(R⁵)₂;
X¹, X², and X⁴ are independently selected from the group consisting of a bond, C(═O), C(═O)N(R⁵), 0, N(R⁵), S, S(O)₂, and S(O)₂N(R⁵);
X³ is O;
R⁵ is selected from the group consisting of H, C₆-C₂₀ aryl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
Q-C(═O)-(PEG)-;
Q-C(═O)-(PEG)-C(═O)—;
Q-C(═O)-(PEG)-O—;
Q-C(═O)-(PEG)-C(═O)N(R⁵)—(C₁-C₁₂ alkyldiyl)-;
Q-C(═O)-(PEG)-C(═O)N(R⁵)—(C₁-C₁₂ alkyldiyl)-N(R⁵)C(═O)—(C₂-C₅ monoheterocyclyldiyl)-; and
Q-C(═O)-(PEG)-N(R⁵)—;
PEG has the formula: —(CH₂CH₂O)ₙ—(CH₂)ₘ—; m is an integer from 1 to 5, and n is an integer from 5 to 20;
Q is selected from the group consisting of N-hydroxysuccinimidyl, N-hydroxysulfosuccinimidyl, and phenoxy substituted with one or more groups independently selected from F, Cl, NO₂, and SO₃—; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)—(CH₂)ₘCO₂H, —O(CH₂CH₂O)₁H, —OP(O)(OH)₂, —S(O)₂N(CH₃)₂, —SCH₃, —S(O)₂CH₃, and —S(O)₃H.

13. The aminobenzazepine-linker compound of claim 12 wherein X¹ is a bond, and R¹ is C₁-C₂₀ heteroaryl.

14. The aminobenzazepine-linker compound of claim 13 wherein R¹ is pyrimidinyl.

15. The aminobenzazepine-linker compound of claim 14 wherein R¹ is pyrimidinyl substituted with —(C₁-C₁₂ alkyldiyl)-N(R⁵)—*.

16. The aminobenzazepine-linker compound of claim 15 wherein —(C₁-C₁₂ alkyldiyl)-N(R⁵)—* is —CH₂—NH—*.

17. The aminobenzazepine-linker compound of claim 16 wherein —CH₂—NH—* is linked at the asterisk to L, wherein L is —C(═O)-(PEG)-C(═O)—.

18. The aminobenzazepine-linker compound of claim 17 wherein PEG has the formula: —(CH₂CH₂O)ₙ—(CH₂)ₘ—; where m is 2 and n is 10.

19. The aminobenzazepine-linker compound of claim 12 wherein X² is a bond and R² is C₁-C₁₂ alkyl.

20. The aminobenzazepine-linker compound of claim 12 wherein R³ is C₁-C₁₂ alkyl.

21. The aminobenzazepine-linker compound of claim 12 wherein X²—R² is —CH₂CH₂CH₃, and X³—R³ is —OCH₂CH₃.

22. The aminobenzazepine-linker compound of claim 12 wherein X⁴ is a bond and R⁴ is H.

23. The aminobenzazepine-linker compound of claim 12 wherein Q is selected from:

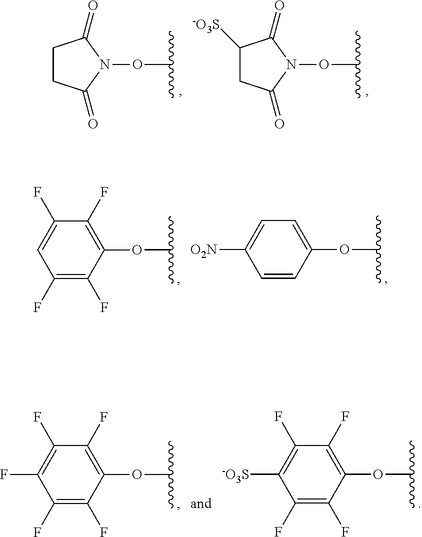

24. A pharmaceutical composition comprising a therapeutically effective amount of an immunoconjugate according to claim 1 and one or more pharmaceutically acceptable diluent, vehicle, carrier or excipient.

25. A method for treating cancer comprising administering a therapeutically effective amount of the pharmaceutical composition according to claim 24 to a patient in need thereof, wherein the cancer is selected from bladder cancer, urinary tract cancer, urothelial carcinoma, lung cancer, non-small cell lung cancer, Merkel cell carcinoma, colon cancer, colorectal cancer, gastric cancer, and breast cancer.

26. The method of claim 25, wherein the cancer is susceptible to a pro-inflammatory response induced by TLR7 and/or TLR8 agonism.

27. A method of preparing an immunoconjugate of Formula I of claim 1 wherein an aminobenzazepine-linker compound of claim 12 is conjugated with the antibody.


SEQUENCE LISTING (608 sequences)

SEQ ID NO: 1 - protein, 5 aa, synthetic construct
  REGION 1..5: note = Description of Artificial Sequence: Synthetic peptide
SYAIS

SEQ ID NO: 2 - protein, 5 aa, synthetic construct
  REGION 1..5: note = Description of Artificial Sequence: Synthetic peptide
SYYMH

SEQ ID NO: 3 - protein, 5 aa, synthetic construct
  REGION 1..5: note = Description of Artificial Sequence: Synthetic peptide
RHLLH

SEQ ID NO: 4 - protein, 5 aa, synthetic construct
  REGION 1..5: note = Description of Artificial Sequence: Synthetic peptide
SHHMH

SEQ ID NO: 5 - protein, 4 aa, synthetic construct
  REGION 1..4: note = Description of Artificial Sequence: Synthetic peptide
RFMH

SEQ ID NO: 6 - protein, 5 aa, synthetic construct
  REGION 1..5: note = Description of Artificial Sequence: Synthetic peptide
SYYIH

SEQ ID NO: 7 - protein, 5 aa, synthetic construct
  REGION 1..5: note = Description of Artificial Sequence: Synthetic peptide
NYYIH

SEQ ID NO: 8 - protein, 5 aa, synthetic construct
  REGION 1..5: note = Description of Artificial Sequence: Synthetic peptide
HYYMH

SEQ ID NO: 9 - protein, 5 aa, synthetic construct
  REGION 1..5: note = Description of Artificial Sequence: Synthetic peptide
GYYMH

SEQ ID NO: 10 - protein, 5 aa, synthetic construct
  REGION 1..5: note = Description of Artificial Sequence: Synthetic peptide
NYMYH

SEQ ID NO: 11 - protein, 5 aa, synthetic construct
  REGION 1..5: note = Description of Artificial Sequence: Synthetic peptide
TYYVH

SEQ ID NO: 12 - protein, 5 aa, synthetic construct
  REGION 1..5: note = Description of Artificial Sequence: Synthetic peptide
SYALS

SEQ ID NO: 13 - protein, 5 aa, synthetic construct
  REGION 1..5: note = Description of Artificial Sequence: Synthetic peptide
RHYVH

SEQ ID NO: 14 - protein, 5 aa, synthetic construct
  REGION 1..5: note = Description of Artificial Sequence: Synthetic peptide
SHYMH

SEQ ID NO: 15 - protein, 5 aa, synthetic construct
  REGION 1..5: note = Description of Artificial Sequence: Synthetic peptide
DYYMH

SEQ ID NO: 16 - protein, 5 aa, synthetic construct
  REGION 1..5: note = Description of Artificial Sequence: Synthetic peptide
GYTLH

SEQ ID NO: 17 - protein, 5 aa, synthetic construct
  REGION 1..5: note = Description of Artificial Sequence: Synthetic peptide
NHYMH

SEQ ID NO: 18 - protein, 5 aa, synthetic construct
  REGION 1..5: note = Description of Artificial Sequence: Synthetic peptide
NSYMH

SEQ ID NO: 19 - protein, 5 aa, synthetic construct
  REGION 1..5: note = Description of Artificial Sequence: Synthetic peptide
TYYMH

SEQ ID NO: 20 - protein, 5 aa, synthetic construct
  REGION 1..5: note = Description of Artificial Sequence: Synthetic peptide
RHFIH

SEQ ID NO: 21 - protein, 5 aa, synthetic construct
  REGION 1..5: note = Description of Artificial Sequence: Synthetic peptide
TFGIS

SEQ ID NO: 22 - protein, 5 aa, synthetic construct
  REGION 1..5: note = Description of Artificial Sequence: Synthetic peptide
SYGIN

SEQ ID NO: 23 - protein, 5 aa, synthetic construct
  REGION 1..5: note = Description of Artificial Sequence: Synthetic peptide
NHYVH

SEQ ID NO: 24 - protein, 17 aa, synthetic construct
  REGION 1..17: note = Description of Artificial Sequence: Synthetic peptide
VINPSAGSTDYAQKFQG

SEQ ID NO: 25 - protein, 17 aa, synthetic construct
  REGION 1..17: note = Description of Artificial Sequence: Synthetic peptide
WMNPNSDIAGYAQKFQG

SEQ ID NO: 26 - protein, 17 aa, synthetic construct
  REGION 1..17: note = Description of Artificial Sequence: Synthetic peptide
WISPQHGVRNYAQKFQG

SEQ ID NO: 27 - protein, 17 aa, synthetic construct
  REGION 1..17: note = Description of Artificial Sequence: Synthetic peptide
WVSPSHGLTGYAQKFQG

SEQ ID NO: 28 - protein, 17 aa, synthetic construct
  REGION 1..17: note = Description of Artificial Sequence: Synthetic peptide
WMSLNSGLTGYAQKFQG

SEQ ID NO: 29 - protein, 17 aa, synthetic construct
  REGION 1..17: note = Description of Artificial Sequence: Synthetic peptide
WMKPSSGTTGYAQKFQG

SEQ ID NO: 30 - protein, 17 aa, synthetic construct
  REGION 1..17: note = Description of Artificial Sequence: Synthetic peptide
WMNPNGDVAGYAQKFQG

SEQ ID NO: 31 - protein, 17 aa, synthetic construct
  REGION 1..17: note = Description of Artificial Sequence: Synthetic peptide
GIDPNSGGTNYAQKFQG

SEQ ID NO: 32 - protein, 17 aa, synthetic construct
  REGION 1..17: note = Description of Artificial Sequence: Synthetic peptide
WMNPDSGSTGYAQKFQG

SEQ ID NO: 33 - protein, 17 aa, synthetic construct
  REGION 1..17: note = Description of Artificial Sequence: Synthetic peptide
WMSLNSGLTGYAQKFQG

SEQ ID NO: 34 - protein, 17 aa, synthetic construct
  REGION 1..17: note = Description of Artificial Sequence: Synthetic peptide
WMNPNGDVAGYADSFQG

SEQ ID NO: 35 - protein, 17 aa, synthetic construct
  REGION 1..17: note = Description of Artificial Sequence: Synthetic peptide
WISTYHGSTNYAQKFQG

SEQ ID NO: 36 - protein, 17 aa, synthetic construct
  REGION 1..17: note = Description of Artificial Sequence: Synthetic peptide
WMNPNTVYTGSAQKFQG

SEQ ID NO: 37 - protein, 17 aa, synthetic construct
  REGION 1..17: note = Description of Artificial Sequence: Synthetic peptide
RIIPAVGSVTYAQKFQG

SEQ ID NO: 38 - protein, 17 aa, synthetic construct
  REGION 1..17: note = Description of Artificial Sequence: Synthetic peptide
GIIPIFGTANYAQKFQG

SEQ ID NO: 39 - protein, 17 aa, synthetic construct
  REGION 1..17: note = Description of Artificial Sequence: Synthetic peptide
WMSPSSGITGYAQKFQG

SEQ ID NO: 40 - protein, 17 aa, synthetic construct
  REGION 1..17: note = Description of Artificial Sequence: Synthetic peptide
WMTPSTGNAGYAQKFQG

SEQ ID NO: 41 - protein, 17 aa, synthetic construct
  REGION 1..17: note = Description of Artificial Sequence: Synthetic peptide
WMNPNSGNTGYAQKFQG

SEQ ID NO: 42 - protein, 17 aa, synthetic construct
  REGION 1..17: note = Description of Artificial Sequence: Synthetic peptide
WMHPNSGHTGYAQKFQG

SEQ ID NO: 43 - protein, 17 aa, synthetic construct
  REGION 1..17: note = Description of Artificial Sequence: Synthetic peptide
WMNPNSGHTGNAQKFQG

SEQ ID NO: 44 - protein, 17 aa, synthetic construct
  REGION 1..17: note = Description of Artificial Sequence: Synthetic peptide
WIDPNSGVTSSAQKFQG

SEQ ID NO: 45 - protein, 17 aa, synthetic construct
  REGION 1..17: note = Description of Artificial Sequence: Synthetic peptide
WISPNSGVTDFTQKFQG

SEQ ID NO: 46 - protein, 17 aa, synthetic construct
  REGION 1..17: note = Description of Artificial Sequence: Synthetic peptide
WMNPNSGHTGYAQRFQG

SEQ ID NO: 47 - protein, 17 aa, synthetic construct
  REGION 1..17: note = Description of Artificial Sequence: Synthetic peptide
WMSPNGGNTGYAQKFQG

SEQ ID NO: 48 - protein, 17 aa, synthetic construct
  REGION 1..17: note = Description of Artificial Sequence: Synthetic peptide
WMDPSSGYTGSAHKFQG

SEQ ID NO: 49 - protein, 17 aa, synthetic construct
  REGION 1..17: note = Description of Artificial Sequence: Synthetic peptide
WMNPHSADTGYAEKFQG

SEQ ID NO: 50 - protein, 17 aa, synthetic construct
  REGION 1..17: note = Description of Artificial Sequence: Synthetic peptide
WLTPSTGHAGYAQKFQG

SEQ ID NO: 51 - protein, 17 aa, synthetic construct
  REGION 1..17: note = Description of Artificial Sequence: Synthetic peptide
WMNPNSGHTGYAQKFQG

SEQ ID NO: 52 - protein, 17 aa, synthetic construct
  REGION 1..17: note = Description of Artificial Sequence: Synthetic peptide
WISPQHGVRNYAHKFQG

SEQ ID NO: 53 - protein, 17 aa, synthetic construct
  REGION 1..17: note = Description of Artificial Sequence: Synthetic peptide
MINPSGGSTSYAQKFQG

SEQ ID NO: 54 - protein, 17 aa, synthetic construct
  REGION 1..17: note = Description of Artificial Sequence: Synthetic peptide
WISPRSGVTSYAQKFQG

SEQ ID NO: 55 - protein, 17 aa, synthetic construct
  REGION 1..17: note = Description of Artificial Sequence: Synthetic peptide
WMDPNSGNTGYAQKFQG

SEQ ID NO: 56 - protein, 17 aa, synthetic construct
  REGION 1..17: note = Description of Artificial Sequence: Synthetic peptide
WMNPTGGITGYAQKFQG

SEQ ID NO: 57 - protein, 17 aa, synthetic construct
  REGION 1..17: note = Description of Artificial Sequence: Synthetic peptide
WVSPIHGLTGYAPRFQG

SEQ ID NO: 58 - protein, 18 aa, synthetic construct
  REGION 1..18: note = Description of Artificial Sequence: Synthetic peptide
DLYPYVVVVAAGSYGMDV

SEQ ID NO: 59 - protein, 12 aa, synthetic construct
  REGION 1..12: note = Description of Artificial Sequence: Synthetic peptide
PSIVGAYDAFDI

SEQ ID NO: 60 - protein, 9 aa, synthetic construct
  REGION 1..9: note = Description of Artificial Sequence: Synthetic peptide
ESVEGYFDL

SEQ ID NO: 61 - protein, 11 aa, synthetic construct
  REGION 1..11: note = Description of Artificial Sequence: Synthetic peptide
DNWNVHDAFDI

SEQ ID NO: 62 - protein, 9 aa, synthetic construct
  REGION 1..9: note = Description of Artificial Sequence: Synthetic peptide
GTYNDAFDI

SEQ ID NO: 63 - protein, 11 aa, synthetic construct
  REGION 1..11: note = Description of Artificial Sequence: Synthetic peptide
EQWLVNDAFDI

SEQ ID NO: 64 - protein, 13 aa, synthetic construct
  REGION 1..13: note = Description of Artificial Sequence: Synthetic peptide
DSSGWMRNDAFDI

SEQ ID NO: 65 - protein, 14 aa, synthetic construct
  REGION 1..14: note = Description of Artificial Sequence: Synthetic peptide
SMFPTIFGDNAFDI

SEQ ID NO: 66 - protein, 14 aa, synthetic construct
  REGION 1..14: note = Description of Artificial Sequence: Synthetic peptide
ALFPYPFYYYYMDV

SEQ ID NO: 67 - protein, 7 aa, synthetic construct
  REGION 1..7: note = Description of Artificial Sequence: Synthetic peptide
DRGWFDP

SEQ ID NO: 68 - protein, 10 aa, synthetic construct
  REGION 1..10: note = Description of Artificial Sequence: Synthetic peptide
DARGYSGYDL

SEQ ID NO: 69 - protein, 9 aa, synthetic construct
  REGION 1..9: note = Description of Artificial Sequence: Synthetic peptide
EGRHGEYLY

SEQ ID NO: 70 - protein, 11 aa, synthetic construct
  REGION 1..11: note = Description of Artificial Sequence: Synthetic peptide
EGWGSSGYFDY

SEQ ID NO: 71 - protein, 15 aa, synthetic construct
  REGION 1..15: note = Description of Artificial Sequence: Synthetic peptide
HLFPTVFDDYYGMDV

SEQ ID NO: 72 - protein, 10 aa, synthetic construct
  REGION 1..10: note = Description of Artificial Sequence: Synthetic peptide
GGYSYGSFQH

SEQ ID NO: 73 - protein, 10 aa, synthetic construct
  REGION 1..10: note = Description of Artificial Sequence: Synthetic peptide
VRWSSDAFDI

SEQ ID NO: 74 - protein, 9 aa, synthetic construct
  REGION 1..9: note = Description of Artificial Sequence: Synthetic peptide
EEWLGHFQH

SEQ ID NO: 75 - protein, 9 aa, synthetic construct
  REGION 1..9: note = Description of Artificial Sequence: Synthetic peptide
ERFLGGMDV

SEQ ID NO: 76 - protein, 9 aa, synthetic construct
  REGION 1..9: note = Description of Artificial Sequence: Synthetic peptide
GNWVDAFDI

SEQ ID NO: 77 - protein, 11 aa, synthetic construct
  REGION 1..11: note = Description of Artificial Sequence: Synthetic peptide
ESEVMMAYFQH

SEQ ID NO: 78 - protein, 9 aa, synthetic construct
  REGION 1..9: note = Description of Artificial Sequence: Synthetic peptide
ESWSGEFDY

SEQ ID NO: 79 - protein, 9 aa, synthetic construct
  REGION 1..9: note = Description of Artificial Sequence: Synthetic peptide
EAVAGPMDV

SEQ ID NO: 80 - protein, 10 aa, synthetic construct
  REGION 1..10: note = Description of Artificial Sequence: Synthetic peptide
DAWELLAFDI

SEQ ID NO: 81 - protein, 10 aa, synthetic construct
  REGION 1..10: note = Description of Artificial Sequence: Synthetic peptide
DRWDGDYYSA

SEQ ID NO: 82 - protein, 10 aa, synthetic construct
  REGION 1..10: note = Description of Artificial Sequence: Synthetic peptide
ESWELTGFDY

SEQ ID NO: 83 - protein, 9 aa, synthetic construct
  REGION 1..9: note = Description of Artificial Sequence: Synthetic peptide
ERFAGGMDA

SEQ ID NO: 84 - protein, 8 aa, synthetic construct
  REGION 1..8: note = Description of Artificial Sequence: Synthetic peptide
DSGGAFDI

SEQ ID NO: 85 - protein, 9 aa, synthetic construct
  REGION 1..9: note = Description of Artificial Sequence: Synthetic peptide
EVFEGGMDV

SEQ ID NO: 86 - protein, 9 aa, synthetic construct
  REGION 1..9: note = Description of Artificial Sequence: Synthetic peptide
EGYGGNYGN

SEQ ID NO: 87 - protein, 9 aa, synthetic construct
  REGION 1..9: note = Description of Artificial Sequence: Synthetic peptide
EDFYGDFDY

SEQ ID NO: 88 - protein, 9 aa, synthetic construct
  REGION 1..9: note = Description of Artificial Sequence: Synthetic peptide
ELSRWGFDY

SEQ ID NO: 89 - protein, 14 aa, synthetic construct
  REGION 1..14: note = Description of Artificial Sequence: Synthetic peptide
DIFPTMIAGGGFDL

SEQ ID NO: 90 - protein, 10 aa, synthetic construct
  REGION 1..10: note = Description of Artificial Sequence: Synthetic peptide
GGYSYGSFDY

SEQ ID NO: 91 - protein, 15 aa, synthetic construct
  REGION 1..15: note = Description of Artificial Sequence: Synthetic peptide
GSFPLVFTIFGVGDV

SEQ ID NO: 92 - protein, 10 aa, synthetic construct
  REGION 1..10: note = Description of Artificial Sequence: Synthetic peptide
DLDYVRAFDI

SEQ ID NO: 93 - protein, 9 aa, synthetic construct
  REGION 1..9: note = Description of Artificial Sequence: Synthetic peptide
ESWGGYFDL

SEQ ID NO: 94 - protein, 9 aa, synthetic construct
  REGION 1..9: note = Description of Artificial Sequence: Synthetic peptide
DRTTYAFDI

SEQ ID NO: 95 - protein, 11 aa, synthetic construct
  REGION 1..11: note = Description of Artificial Sequence: Synthetic peptide
VHGSGSDGMDV

SEQ ID NO: 96 - protein, 11 aa, synthetic construct
  REGION 1..11: note = Description of Artificial Sequence: Synthetic peptide
RASQGIDSYLA

SEQ ID NO: 97 - protein, 11 aa, synthetic construct
  REGION 1..11: note = Description of Artificial Sequence: Synthetic peptide
RASQSISSWLA

SEQ ID NO: 98 - protein, 11 aa, synthetic construct
  REGION 1..11: note = Description of Artificial Sequence: Synthetic peptide
RASQSISSYLN

SEQ ID NO: 99 - protein, 11 aa, synthetic construct
  REGION 1..11: note = Description of Artificial Sequence: Synthetic peptide
RASQGISSYLA

SEQ ID NO: 100 - protein, 11 aa, synthetic construct
  REGION 1..11: note = Description of Artificial Sequence: Synthetic peptide
RASQTISNYLN

SEQ ID NO: 101 - protein, 12 aa, synthetic construct
  REGION 1..12: note = Description of Artificial Sequence: Synthetic peptide
RASQSVDRNYVT

SEQ ID NO: 102 - protein, 11 aa, synthetic construct
  REGION 1..11: note = Description of Artificial Sequence: Synthetic peptide
RASQGISQYLA

SEQ ID NO: 103 - protein, 11 aa, synthetic construct
  REGION 1..11: note = Description of Artificial Sequence: Synthetic peptide
QASQDIGNYLN

SEQ ID NO: 104 - protein, 11 aa, synthetic construct
  REGION 1..11: note = Description of Artificial Sequence: Synthetic peptide
RASQGIRNDLG

SEQ ID NO: 105 - protein, 11 aa, synthetic construct
  REGION 1..11: note = Description of Artificial Sequence: Synthetic peptide
RASQIIGNYLA

SEQ ID NO: 106 - protein, 11 aa, synthetic construct
  REGION 1..11: note = Description of Artificial Sequence: Synthetic peptide
RASQIISSYLN

SEQ ID NO: 107 - protein, 11 aa, synthetic construct
  REGION 1..11: note = Description of Artificial Sequence: Synthetic peptide
QASQDISNYLN

SEQ ID NO: 108 - protein, 11 aa, synthetic construct
  REGION 1..11: note = Description of Artificial Sequence: Synthetic peptide
RASQGISNYLA

SEQ ID NO: 109 - protein, 11 aa, synthetic construct
  REGION 1..11: note = Description of Artificial Sequence: Synthetic peptide
RASQGISNNLN

SEQ ID NO: 110 - protein, 11 aa, synthetic construct
  REGION 1..11: note = Description of Artificial Sequence: Synthetic peptide
RASQGISNGLS

SEQ ID NO: 111 - protein, 11 aa, synthetic construct
  REGION 1..11: note = Description of Artificial Sequence: Synthetic peptide
RASQSITGWLA

SEQ ID NO: 112 - protein, 11 aa, synthetic construct
  REGION 1..11: note = Description of Artificial Sequence: Synthetic peptide
RASQGIRNDLA

SEQ ID NO: 113 - protein, 11 aa, synthetic construct
  REGION 1..11: note = Description of Artificial Sequence: Synthetic peptide
QASQDISSYLN

SEQ ID NO: 114 - protein, 11 aa, synthetic construct
  REGION 1..11: note = Description of Artificial Sequence: Synthetic peptide
RASQSITTYLN

SEQ ID NO: 115 - protein, 11 aa, synthetic construct
  REGION 1..11: note = Description of Artificial Sequence: Synthetic peptide
RASQSVSTWLA

SEQ ID NO: 116 - protein, 11 aa, synthetic construct
  REGION 1..11: note = Description of Artificial Sequence: Synthetic peptide
RASQSISNWLA

SEQ ID NO: 117 - protein, 11 aa, synthetic construct
  REGION 1..11: note = Description of Artificial Sequence: Synthetic peptide
QASQGISNYLA

SEQ ID NO: 118 - protein, 12 aa, synthetic construct
  REGION 1..12: note = Description of Artificial Sequence: Synthetic peptide
RASQSLSSSSLA

SEQ ID NO: 119 - protein, 11 aa, synthetic construct
  REGION 1..11: note = Description of Artificial Sequence: Synthetic peptide
RASEHIANWLA

SEQ ID NO: 120 - protein, 11 aa, synthetic construct
  REGION 1..11: note = Description of Artificial Sequence: Synthetic peptide
RASQSVGSWVA

SEQ ID NO: 121 - protein, 11 aa, synthetic construct
  REGION 1..11: note = Description of Artificial Sequence: Synthetic peptide
RASQSISPWLA

SEQ ID NO: 122 - protein, 11 aa, synthetic construct
  REGION 1..11: note = Description of Artificial Sequence: Synthetic peptide
RASQGISRYLA

SEQ ID NO: 123 - protein, 12 aa, synthetic construct
  REGION 1..12: note = Description of Artificial Sequence: Synthetic peptide
RASQTVSSNYLA

SEQ ID NO: 124 - protein, 11 aa, synthetic construct
  REGION 1..11: note = Description of Artificial Sequence: Synthetic peptide
RSSQGIRNDLS

SEQ ID NO: 125 - protein, 11 aa, synthetic construct
  REGION 1..11: note = Description of Artificial Sequence: Synthetic peptide
RDSHSITTWLA

SEQ ID NO: 126 - protein, 11 aa, synthetic construct
  REGION 1..11: note = Description of Artificial Sequence: Synthetic peptide
RASQSISRWLA

SEQ ID NO: 127 - protein, 11 aa, synthetic construct
  REGION 1..11: note = Description of Artificial Sequence: Synthetic peptide
RASQVIRNDLA

SEQ ID NO: 128 - skipped

SEQ ID NO: 129 - protein, 7 aa, synthetic construct
  REGION 1..7: note = Description of Artificial Sequence: Synthetic peptide
AASSLQS

SEQ ID NO: 130 - protein, 7 aa, synthetic construct
  REGION 1..7: note = Description of Artificial Sequence: Synthetic peptide
GASNLQS

SEQ ID NO: 131 - protein, 7 aa, synthetic construct
  REGION 1..7: note = Description of Artificial Sequence: Synthetic peptide
GASTRAT

SEQ ID NO: 132 - protein, 7 aa, synthetic construct
  REGION 1..7: note = Description of Artificial Sequence: Synthetic peptide
GASNLHS

SEQ ID NO: 133 - protein, 7 aa, synthetic construct
  REGION 1..7: note = Description of Artificial Sequence: Synthetic peptide
AASSLES

SEQ ID NO: 134 - protein, 7 aa, synthetic construct
  REGION 1..7: note = Description of Artificial Sequence: Synthetic peptide
SASNLQS

SEQ ID NO: 135 - protein, 7 aa, synthetic construct
  REGION 1..7: note = Description of Artificial Sequence: Synthetic peptide
AASTLES

SEQ ID NO: 136 - protein, 7 aa, synthetic construct
  REGION 1..7: note = Description of Artificial Sequence: Synthetic peptide
HASILET

SEQ ID NO: 137 - protein, 7 aa, synthetic construct
  REGION 1..7: note = Description of Artificial Sequence: Synthetic peptide
AASTLQS

SEQ ID NO: 138 - protein, 7 aa, synthetic construct
  REGION 1..7: note = Description of Artificial Sequence: Synthetic peptide
AATTLQS

SEQ ID NO: 139 - protein, 7 aa, synthetic construct
  REGION 1..7: note = Description of Artificial Sequence: Synthetic peptide
DATHLET

SEQ ID NO: 140 - protein, 7 aa, synthetic construct
  REGION 1..7: note = Description of Artificial Sequence: Synthetic peptide
AASSLQT

SEQ ID NO: 141 - protein, 7 aa, synthetic construct
  REGION 1..7: note = Description of Artificial Sequence: Synthetic peptide
AASSLQG

SEQ ID NO: 142 - protein, 7 aa, synthetic construct
  REGION 1..7: note = Description of Artificial Sequence: Synthetic peptide
AASNLES

SEQ ID NO: 143 - protein, 7 aa, synthetic construct
  REGION 1..7: note = Description of Artificial Sequence: Synthetic peptide
DVSHLES

SEQ ID NO: 144 - protein, 7 aa, synthetic construct
  REGION 1..7: note = Description of Artificial Sequence: Synthetic peptide
DASSLQS

SEQ ID NO: 145 - protein, 7 aa, synthetic construct
  REGION 1..7: note = Description of Artificial Sequence: Synthetic peptide
GVSSLES

SEQ ID NO: 146 - protein, 7 aa, synthetic construct
  REGION 1..7: note = Description of Artificial Sequence: Synthetic peptide
PASTLQS

SEQ ID NO: 147 - protein, 7 aa, synthetic construct
  REGION 1..7: note = Description of Artificial Sequence: Synthetic peptide
DASNLET

SEQ ID NO: 148 - protein, 7 aa, synthetic construct
  REGION 1..7: note = Description of Artificial Sequence: Synthetic peptide
GASTRAS

SEQ ID NO: 149 - protein, 7 aa, synthetic construct
  REGION 1..7: note = Description of Artificial Sequence: Synthetic peptide
DSSSLQT

SEQ ID NO: 150 - protein, 7 aa, synthetic construct
  REGION 1..7: note = Description of Artificial Sequence: Synthetic peptide
ATSTLQS

SEQ ID NO: 151 - protein, 7 aa, synthetic construct
  REGION 1..7: note = Description of Artificial Sequence: Synthetic peptide
LASNSHS

SEQ ID NO: 152 - protein, 9 aa, synthetic construct
  REGION 1..9: note = Description of Artificial Sequence: Synthetic peptide
QQSYSTPIT

SEQ ID NO: 153 - protein, 9 aa, synthetic construct
  REGION 1..9: note = Description of Artificial Sequence: Synthetic peptide
QQSYTTPIT

SEQ ID NO: 154 - protein, 9 aa, synthetic construct
  REGION 1..9: note = Description of Artificial Sequence: Synthetic peptide
QQIFSTPLT

SEQ ID NO: 155 - protein, 9 aa, synthetic construct
  REGION 1..9: note = Description of Artificial Sequence: Synthetic peptide
QQSYSTPLT

SEQ ID NO: 156 - protein, 9 aa, synthetic construct
  REGION 1..9: note = Description of Artificial Sequence: Synthetic peptide
QQSYTTPYT

SEQ ID NO: 157 - protein, 9 aa, synthetic construct
  REGION 1..9: note = Description of Artificial Sequence: Synthetic peptide
QQTFTTPLT

SEQ ID NO: 158 - protein, 9 aa, synthetic construct
  REGION 1..9: note = Description of Artificial Sequence: Synthetic peptide
QQANSFPFT

SEQ ID NO: 159 - protein, 9 aa, synthetic construct
  REGION 1..9: note = Description of Artificial Sequence: Synthetic peptide
QQSYTTPYS

SEQ ID NO: 160 - protein, 9 aa, synthetic construct
  REGION 1..9: note = Description of Artificial Sequence: Synthetic peptide
QQTFITPLT

SEQ ID NO: 161 - protein, 8 aa, synthetic construct
  REGION 1..8: note = Description of Artificial Sequence: Synthetic peptide
QQSYSTPT

SEQ ID NO: 162 - protein, 9 aa, synthetic construct
  REGION 1..9: note = Description of Artificial Sequence: Synthetic peptide
QQGFSTPFT

SEQ ID NO: 163 - protein, 9 aa, synthetic construct
  REGION 1..9: note = Description of Artificial Sequence: Synthetic peptide
QQSFTNPVT

SEQ ID NO: 164 - protein, 9 aa, synthetic construct
  REGION 1..9: note = Description of Artificial Sequence: Synthetic peptide
QQSYSAPYT

SEQ ID NO: 165 - protein, 9 aa, synthetic construct
  REGION 1..9: note = Description of Artificial Sequence: Synthetic peptide
QQSYSTPYT

SEQ ID NO: 166 - protein, 9 aa, synthetic construct
  REGION 1..9: note = Description of Artificial Sequence: Synthetic peptide
QQSHSTPLT

SEQ ID NO: 167 - protein, 9 aa, synthetic construct
  REGION 1..9: note = Description of Artificial Sequence: Synthetic peptide
QQSYSTPFT

SEQ ID NO: 168 - protein, 9 aa, synthetic construct
  REGION 1..9: note = Description of Artificial Sequence: Synthetic peptide
QQYGSSPFT

SEQ ID NO: 169 - protein, 9 aa, synthetic construct
  REGION 1..9: note = Description of Artificial Sequence: Synthetic peptide
QQTYSTPIT

SEQ ID NO: 170 - protein, 9 aa, synthetic construct
  REGION 1..9: note = Description of Artificial Sequence: Synthetic peptide
QQYYTTPLT

SEQ ID NO: 171 - protein, 9 aa, synthetic construct
  REGION 1..9: note = Description of Artificial Sequence: Synthetic peptide
QQSFSTPLT

SEQ ID NO: 172 - protein, 9 aa, synthetic construct
  REGION 1..9: note = Description of Artificial Sequence: Synthetic peptide
QQSYSTPPT

SEQ ID NO: 173 - protein, 9 aa, synthetic construct
  REGION 1..9: note = Description of Artificial Sequence: Synthetic peptide
LQHNSYPLT

SEQ ID NO: 174 - protein, 9 aa, synthetic construct
  REGION 1..9: note = Description of Artificial Sequence: Synthetic peptide
QQSYSTPVT

SEQ ID NO: 175 - protein, 9 aa, synthetic construct
  REGION 1..9: note = Description of Artificial Sequence: Synthetic peptide
QHFYNTQYT

SEQ ID NO: 176 - protein, 10 aa, synthetic construct
  REGION 1..10: note = Description of Artificial Sequence: Synthetic peptide
QQSLQYPSHF

SEQ ID NO: 177 - protein, 30 aa, synthetic construct
  REGION 1..30: note = Description of Artificial Sequence: Synthetic polypeptide
QVQLVQSGAEVKKPGASVKVSCKASGGTFS

SEQ ID NO: 178 - protein, 30 aa, synthetic construct
  REGION 1..30: note = Description of Artificial Sequence: Synthetic polypeptide
QVQLVQSGAEVKKPGASVKVSCKASGYTFT

SEQ ID NO: 179 - protein, 30 aa, synthetic construct
  REGION 1..30: note = Description of Artificial Sequence: Synthetic polypeptide
QVQLVQSGAEVKKPGASVKVSCKASGYTFN

SEQ ID NO: 180 - protein, 30 aa, synthetic construct
  REGION 1..30: note = Description of Artificial Sequence: Synthetic polypeptide
QVQLVQSGAEVKKPGASVKVSCKASGNTFT

SEQ ID NO: 181 - protein, 30 aa, synthetic construct
  REGION 1..30: note = Description of Artificial Sequence: Synthetic polypeptide
QVQLVQSGAEVKKPGASVKVSCKASGHSFT

SEQ ID NO: 182 - protein, 30 aa, synthetic construct
  REGION 1..30: note = Description of Artificial Sequence: Synthetic polypeptide
QVQLVQSGAEVKKPGASVKVSCKASGYPFT

SEQ ID NO: 183 - protein, 30 aa, synthetic construct
  REGION 1..30: note = Description of Artificial Sequence: Synthetic polypeptide
QVQLVQSGAEVKKPGASVKVSCKASGYRFT

SEQ ID NO: 184 - protein, 30 aa, synthetic construct
  REGION 1..30: note = Description of Artificial Sequence: Synthetic polypeptide
QVQLVQSGAEVKKPGSSVKVSCKASGGTFS

SEQ ID NO: 185 - protein, 30 aa, synthetic construct
  REGION 1..30: note = Description of Artificial Sequence: Synthetic polypeptide
QVQLVQSGAEVKKPGASVKVSCKASGDTFT

SEQ ID NO: 186 - protein, 30 aa, synthetic construct
  REGION 1..30: note = Description of Artificial Sequence: Synthetic polypeptide
QVQLAQSGAEVKKPGASVKVSCKASGYTFT

SEQ ID NO: 187 - protein, 30 aa, synthetic construct
  REGION 1..30: note = Description of Artificial Sequence: Synthetic polypeptide
QVQLVQSGAEVKKPGASVKVSCKASGYTFS

SEQ ID NO: 188 - protein, 30 aa, synthetic construct
  REGION 1..30: note = Description of Artificial Sequence: Synthetic polypeptide
QVQLVQSGAEVKKPGASVKVSCKASGYPFS

SEQ ID NO: 189 - protein, 30 aa, synthetic construct
  REGION 1..30: note = Description of Artificial Sequence: Synthetic polypeptide
QVQLVQSGAEVKKPGASVKVSCKASGYSFT

SEQ ID NO: 190 - protein, 30 aa, synthetic construct
  REGION 1..30: note = Description of Artificial Sequence: Synthetic polypeptide
QVQLVQSGAEVKKPGASVKVSCKASGYTFI

SEQ ID NO: 191 - protein, 30 aa, synthetic construct
  REGION 1..30: note = Description of Artificial Sequence: Synthetic polypeptide
QVQLVQSGAEVKKPGSSVKVSCKASGYTFT

SEQ ID NO: 192 - protein, 14 aa, synthetic construct
  REGION 1..14: note = Description of Artificial Sequence: Synthetic peptide
WVRQAPGQGLEWMG

SEQ ID NO: 193 - protein, 14 aa, synthetic construct
  REGION 1..14: note = Description of Artificial Sequence: Synthetic peptide
WVRQAPGQGLEWLG

SEQ ID NO: 194 - protein, 14 aa, synthetic construct
  REGION 1..14: note = Description of Artificial Sequence: Synthetic peptide
WVRQAPGQGLEWIG

SEQ ID NO: 195 - protein, 14 aa, synthetic construct
  REGION 1..14: note = Description of Artificial Sequence: Synthetic peptide
WVRQAPGQGLEWVG

SEQ ID NO: 196 - protein, 32 aa, synthetic construct
  REGION 1..32: note = Description of Artificial Sequence: Synthetic polypeptide
RVTMTRDTSTSTVYMELSSLRSEDTAVYYCAR

SEQ ID NO: 197 - protein, 32 aa, synthetic construct
  REGION 1..32: note = Description of Artificial Sequence: Synthetic polypeptide
RVTMTRDTSTSTVYMELSSLRSEDTAVYYCAI

SEQ ID NO: 198 - protein, 32 aa, synthetic construct
  REGION 1..32: note = Description of Artificial Sequence: Synthetic polypeptide
RVTMTRDTSTSTVYMELSSLRSEDTAVYYCTR

SEQ ID NO: 199 - protein, 32 aa, synthetic construct
  REGION 1..32: note = Description of Artificial Sequence: Synthetic polypeptide
RVTITADESTSTAYMELSSLRSEDTAVYYCAR

SEQ ID NO: 200 - protein, 32 aa, synthetic construct
  REGION 1..32: note = Description of Artificial Sequence: Synthetic polypeptide
RVTMTRDTSTSTVNMELSSLRSEDTAVYYCAR

SEQ ID NO: 201 - protein, 32 aa, synthetic construct
  REGION 1..32: note = Description of Artificial Sequence: Synthetic polypeptide
RVTMTRDTSTSTVYMELSSLRSEDTAVYYCAE

SEQ ID NO: 202 - protein, 11 aa, synthetic construct
  REGION 1..11: note = Description of Artificial Sequence: Synthetic peptide
WGQGTLVTVSS

SEQ ID NO: 203 - protein, 11 aa, synthetic construct
  REGION 1..11: note = Description of Artificial Sequence: Synthetic peptide
WGQGTTVTVSS

SEQ ID NO: 204 - protein, 11 aa, synthetic construct
  REGION 1..11: note = Description of Artificial Sequence: Synthetic peptide
WGQGTMVTVSS

SEQ ID NO: 205 - protein, 11 aa, synthetic construct
  REGION 1..11: note = Description of Artificial Sequence: Synthetic peptide
WGPGTMVTVSS

SEQ ID NO: 206 - protein, 11 aa, synthetic construct
  REGION 1..11: note = Description of Artificial Sequence: Synthetic peptide
WGRGTLVTVSS

SEQ ID NO: 207 - protein, 23 aa, synthetic construct
  REGION 1..23: note = Description of Artificial Sequence: Synthetic peptide
DIQMTQSPSSLSASVGDRVTITC

SEQ ID NO: 208 - protein, 23 aa, synthetic construct
  REGION 1..23: note = Description of Artificial Sequence: Synthetic peptide
EIVMTQSPATLSVSPGERATLSC

SEQ ID NO: 209 - protein, 23 aa, synthetic construct
  REGION 1..23: note = Description of Artificial Sequence: Synthetic peptide
DIQITHSPSSLSASVGYRLTITC

SEQ ID NO: 210 - protein, 15 aa, synthetic construct
  REGION 1..15: note = Description of Artificial Sequence: Synthetic peptide
WYQQKPGKAPKLLIY

SEQ ID NO: 211 - protein, 15 aa, synthetic construct
  REGION 1..15: note = Description of Artificial Sequence: Synthetic peptide
WYQQKPGQAPRLLIY

SEQ ID NO: 212 - protein, 15 aa, synthetic construct
  REGION 1..15: note = Description of Artificial Sequence: Synthetic peptide
WYHQKPWNAPKLMIY

SEQ ID NO: 213 - protein, 32 aa, synthetic construct
  REGION 1..32: note = Description of Artificial Sequence: Synthetic polypeptide
GVPSRFSGSGSGTDFTLTISSLQPEDFATYYC

SEQ ID NO: 214 - protein, 32 aa, synthetic construct
  REGION 1..32: note = Description of Artificial Sequence: Synthetic polypeptide
GIPARFSGSGSGTEFTLTISSLQSEDFAVYYC

SEQ ID NO: 215 - protein, 32 aa, synthetic construct
  REGION 1..32: note = Description of Artificial Sequence: Synthetic polypeptide
GVPSRFSGSGSGTYFTLTISSLQPEDFATYYC

SEQ ID NO: 216 - protein, 32 aa, synthetic construct
  REGION 1..32: note = Description of Artificial Sequence: Synthetic polypeptide
GVPSRFSGSGSGTDFTLTISSLQPEDFAPYYC

SEQ ID NO: 217 - protein, 10 aa, synthetic construct
  REGION 1..10: note = Description of Artificial Sequence: Synthetic peptide
FGGGTKVEIK

SEQ ID NO: 218 - protein, 10 aa, synthetic construct
  REGION 1..10: note = Description of Artificial Sequence: Synthetic peptide
FGQGTKVEIK

SEQ ID NO: 219 - protein, 10 aa, synthetic construct
  REGION 1..10: note = Description of Artificial Sequence: Synthetic peptide
FGQGTRLEIK

SEQ ID NO: 220 - protein, 10 aa, synthetic construct
  REGION 1..10: note = Description of Artificial Sequence: Synthetic peptide
FGGGTKLEIK

SEQ ID NO: 221 - protein, 10 aa, synthetic construct
  REGION 1..10: note = Description of Artificial Sequence: Synthetic peptide
FGQGTKLEIK

SEQ ID NO: 222 - protein, 10 aa, synthetic construct
  REGION 1..10: note = Description of Artificial Sequence: Synthetic peptide
FGPGTKVDIK

SEQ ID NO: 223 - protein, 127 aa, synthetic construct
  REGION 1..127: note = Description of Artificial Sequence: Synthetic polypeptide
QVQLVQSGAEVKKPGASVKVSCKASGGTFSSYAISWVRQAPGQGLEWMGVINPSAGSTDYAQKFQGRVTMTRDTSTSTVYMELSSLRSEDTAVYYCARDLYPYVVVVAAGSYGMDVWGQGTLVTVSS

SEQ ID NO: 224 - protein, 121 aa, synthetic construct
  REGION 1..121: note = Description of Artificial Sequence: Synthetic polypeptide
QVQLVQSGAEVKKPGASVKVSCKASGYTFTSYYMHWVRQAPGQGLEWMGWMNPNSDIAGYAQKFQGRVTMTRDTSTSTVYMELSSLRSEDTAVYYCAIPSIVGAYDAFDIWGQGTLVTVSS

SEQ ID NO: 225 - protein, 118 aa, synthetic construct
  REGION 1..118: note = Description of Artificial Sequence: Synthetic polypeptide
QVQLVQSGAEVKKPGASVKVSCKASGYTFTRHLLHWVRQAPGQGLEWMGWISPQHGVRNYAQKFQGRVTMTRDTSTSTVYMELSSLRSEDTAVYYCARESVEGYFDLWGQGTLVTVSS

SEQ ID NO: 226 - protein, 120 aa, synthetic construct
  REGION 1..120: note = Description of Artificial Sequence: Synthetic polypeptide
QVQLVQSGAEVKKPGASVKVSCKASGYTFTSHHMHWVRQAPGQGLEWMGWVSPSHGLTGYAQKFQGRVTMTRDTSTSTVYMELSSLRSEDTAVYYCARDNWNVHDAFDIWGQGTLVTVSS

SEQ ID NO: 227 - protein, 117 aa, synthetic construct
  REGION 1..117: note = Description of Artificial Sequence: Synthetic polypeptide
QVQLVQSGAEVKKPGASVKVSCKASGYTFNRFMHWVRQAPGQGLEWMGWMSLNSGLTGYAQKFQGRVTMTRDTSTSTVYMELSSLRSEDTAVYYCTRGTYNDAFDIWGQGTLVTVSS

SEQ ID NO: 228 - protein, 120 aa, synthetic construct
  REGION 1..120: note = Description of Artificial Sequence: Synthetic polypeptide
QVQLVQSGAEVKKPGASVKVSCKASGYTFTSYYIHWVRQAPGQGLEWMGWMKPSSGTTGYAQKFQGRVTMTRDTSTSTVYMELSSLRSEDTAVYYCAREQWLVNDAFDIWGQGTLVTVSS

SEQ ID NO: 229 - protein, 120 aa, synthetic construct; identical to SEQ ID NO: 228
  REGION 1..120: note = Description of Artificial Sequence: Synthetic polypeptide

SEQ ID NO: 230 - protein, 123 aa, synthetic construct
  REGION 1..123: note = Description of Artificial Sequence: Synthetic polypeptide
QVQLVQSGAEVKKPGASVKVSCKASGNTFTSYYMHWVRQAPGQGLEWMGGIDPNSGGTNYAQKFQGRVTMTRDTSTSTVYMELSSLRSEDTAVYYCARSMFPTIFGDNAFDIWGQGTLVTVSS

SEQ ID NO: 231 - protein, 123 aa, synthetic construct
  REGION 1..123: note = Description of Artificial Sequence: Synthetic polypeptide
QVQLVQSGAEVKKPGASVKVSCKASGHSFTHYYMHWVRQAPGQGLEWMGWMNPDSGSTGYAQKFQGRVTMTRDTSTSTVYMELSSLRSEDTAVYYCARALFPYPFYYYYMDVWGQGTLVTVSS

SEQ ID NO: 232 - protein, 116 aa, synthetic construct
  REGION 1..116: note = Description of Artificial Sequence: Synthetic polypeptide
QVQLVQSGAEVKKPGASVKVSCKASGYTFTGYYMHWVRQAPGQGLEWIGWMSLNSGLTGYAQKFQGRVTMTRDTSTSTVYMELSSLRSEDTAVYYCARDRGWFDPWGQGTLVTVSS

SEQ ID NO: 233 - protein, 122 aa, synthetic construct
  REGION 1..122: note = Description of Artificial Sequence: Synthetic polypeptide
QVQLVQSGAEVKKPGASVKVSCKASGYTFTNYYIHWVRQAPGQGLEWLGWMNPNGDVAGYADSFQGRVTMTRDTSTSTVYMELSSLRSEDTAVYYCAIDSSGWMRNDAFDIWGQGTLVTVSS

SEQ ID NO: 234 - protein, 119 aa, synthetic construct
  REGION 1..119: note = Description of Artificial Sequence: Synthetic polypeptide
QVQLVQSGAEVKKPGASVKVSCKASGGTFSNYMYHWVRQAPGQGLEWMGWISTYHGSTNYAQKFQGRVTMTRDTSTSTVYMELSSLRSEDTAVYYCARDARGYSGYDLWGQGTLVTVSS

SEQ ID NO: 235 - protein, 118 aa, synthetic construct
  REGION 1..118: note = Description of Artificial Sequence: Synthetic polypeptide
QVQLVQSGAEVKKPGASVKVSCKASGYPFTSYYMHWVRQAPGQGLEWMGWMNPNSDIAGYAQKFQGRVTMTRDTSTSTVYMELSSLRSEDTAVYYCAREGRHGEYLYWGQGTLVTVSS

SEQ ID NO: 236 - protein, 120 aa, synthetic construct
  REGION 1..120: note = Description of Artificial Sequence: Synthetic polypeptide
QVQLVQSGAEVKKPGASVKVSCKASGYRFTTYYVHWVRQAPGQGLEWMGWMNPNTVYTGSAQKFQGRVTMTRDTSTSTVYMELSSLRSEDTAVYYCAREGWGSSGYFDYWGQGTLVTVSS

SEQ ID NO: 237 - protein, 124 aa, synthetic construct
  REGION 1..124: note = Description of Artificial Sequence: Synthetic polypeptide
QVQLVQSGAEVKKPGSSVKVSCKASGGTFSSYALSWVRQAPGQGLEWMGRIIPAVGSVTYAQKFQGRVTITADESTSTAYMELSSLRSEDTAVYYCARHLFPTVFDDYYGMDVWGQGTLVTVSS

SEQ ID NO: 238 - protein, 119 aa, synthetic construct
  REGION 1..119: note = Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGYSYGSFQHWGQGTLVTVSS

SEQ ID NO: 239 - protein, 119 aa, synthetic construct
  REGION 1..119: note = Description of Artificial Sequence: Synthetic polypeptide
QVQLVQSGAEVKKPGASVKVSCKASGDTFTRHYVHWVRQAPGQGLEWMGWMSPSSGITGYAQKFQGRVTMTRDTSTSTVYMELSSLRSEDTAVYYCARVRWSSDAFDIWGQGTLVTVSS

SEQ ID NO: 240 - protein, 118 aa, synthetic construct
  REGION 1..118: note = Description of Artificial Sequence: Synthetic polypeptide
QVQLVQSGAEVKKPGASVKVSCKASGYTFTSYYMHWVRQAPGQGLEWVGWMTPSTGNAGYAQKFQGRVTMTRDTSTSTVYMELSSLRSEDTAVYYCAREEWLGHFQHWGQGTLVTVSS

SEQ ID NO: 241 - protein, 118 aa, synthetic construct
  REGION 1..118: note = Description of Artificial Sequence: Synthetic polypeptide
QVQLVQSGAEVKKPGASVKVSCKASGYTFTSHYMHWVRQAPGQGLEWMGWMNPNSGNTGYAQKFQGRVTMTRDTSTSTVYMELSSLRSEDTAVYYCARERFLGGMDVWGQGTTVTVSS

SEQ ID NO: 242 - protein, 118 aa, synthetic construct
  REGION 1..118: note = Description of Artificial Sequence: Synthetic polypeptide
QVQLVQSGAEVKKPGASVKVSCKASGYTFTDYYMHWVRQAPGQGLEWMGWMHPNSGHTGYAQKFQGRVTMTRDTSTSTVNMELSSLRSEDTAVYYCAREEWLGHFQHWGQGTLVTVSS

SEQ ID NO: 243 - protein, 118 aa, synthetic construct
  REGION 1..118: note = Description of Artificial Sequence: Synthetic polypeptide
QVQLVQSGAEVKKPGASVKVSCKASGYTFTSHYMHWVRQAPGQGLEWMGWMNPNSGHTGNAQKFQGRVTMTRDTSTSTVYMELSSLRSEDTAVYYCARGNWVDAFDIWGQGTMVTVSS

SEQ ID NO: 244 - protein, 120 aa, synthetic construct
  REGION 1..120: note = Description of Artificial Sequence: Synthetic polypeptide
QVQLVQSGAEVKKPGASVKVSCKASGYTFTGYTLHWVRQAPGQGLEWMGWIDPNSGVTSSAQKFQGRVTMTRDTSTSTVYMELSSLRSEDTAVYYCARESEVMMAYFQHWGQGTLVTVSS

SEQ ID NO: 245 - protein, 118 aa, synthetic construct
  REGION 1..118: note = Description of Artificial Sequence: Synthetic polypeptide
QVQLVQSGAEVKKPGASVKVSCKASGYTFTGYYMHWVRQAPGQGLEWMGWISPNSGVTDFTQKFQGRVTMTRDTSTSTVYMELSSLRSEDTAVYYCARESWSGEFDYWGQGTLVTVSS

SEQ ID NO: 246 - protein, 118 aa, synthetic construct
  REGION 1..118: note = Description of Artificial Sequence: Synthetic polypeptide
QVQLVQSGAEVKKPGASVKVSCKASGDTFTNHYMHWVRQAPGQGLEWMGWMNPNSGHTGYAQRFQGRVTMTRDTSTSTVYMELSSLRSEDTAVYYCAREAVAGPMDVWGQGTTVTVSS

SEQ ID NO: 247 - protein, 119 aa, synthetic construct
  REGION 1..119: note = Description of Artificial Sequence: Synthetic polypeptide
QVQLVQSGAEVKKPGASVKVSCKASGYTFTGYYMHWVRQAPGQGLEWMGWMNPNSDIAGYAQKFQGRVTMTRDTSTSTVYMELSSLRSEDTAVYYCARDAWELLAFDIWGQGTLVTVSS

SEQ ID NO: 248 - protein, 119 aa, synthetic construct
  REGION 1..119: note = Description of Artificial Sequence: Synthetic polypeptide
QVQLAQSGAEVKKPGASVKVSCKASGYTFTNHYMHWVRQAPGQGLEWMGWMNPNSGNTGYAQKFQGRVTMTRDTSTSTVYMELSSLRSEDTAVYYCARDRWDGDYYSAWGQGTLVTVSS

SEQ ID NO: 249 - protein, 119 aa, synthetic construct
  REGION 1..119: note = Description of Artificial Sequence: Synthetic polypeptide
QVQLVQSGAEVKKPGASVKVSCKASGYTFTNYYIHWVRQAPGQGLEWMGWMSPNGGNTGYAQKFQGRVTMTRDTSTSTVYMELSSLRSEDTAVYYCARESWELTGFDYWGQGTLVTVSS

SEQ ID NO: 250 - protein, 118 aa, synthetic construct
  REGION 1..118: note = Description of Artificial Sequence: Synthetic polypeptide
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARERFAGGMDAWGQGTTVTVSS

SEQ ID NO: 251 - protein, 117 aa, synthetic construct
  REGION 1..117: note = Description of Artificial Sequence: Synthetic polypeptide
QVQLVQSGAEVKKPGASVKVSCKASGYTFSNSYMHWVRQAPGQGLEWMGWMDPSSGYTGSAHKFQGRVTMTRDTSTSTVYMELSSLRSEDTAVYYCAEDSGGAFDIWGQGTMVTVSS

SEQ ID NO: 252 - protein, 118 aa, synthetic construct
  REGION 1..118: note = Description of Artificial Sequence: Synthetic polypeptide
QVQLVQSGAEVKKPGASVKVSCKASGYPFSTYYMHWVRQAPGQGLEWMGWMNPHSADTGYAEKFQGRVTMTRDTSTSTVYMELSSLRSEDTAVYYCAREVFEGGMDVWGQGTTVTVSS

SEQ ID NO: 253 - protein, 118 aa, synthetic construct
  REGION 1..118: note = Description of Artificial Sequence: Synthetic polypeptide
QVQLVQSGAEVKKPGASVKVSCKASGYTFTSYYMHWVRQAPGQGLEWMGWLTPSTGHAGYAQKFQGRVTMTRDTSTSTVYMELSSLRSEDTAVYYCAREGYGGNYGNWGQGTLVTVSS

SEQ ID NO: 254 - protein, 118 aa, synthetic construct
  REGION 1..118: note = Description of Artificial Sequence: Synthetic polypeptide
QVQLVQSGAEVKKPGASVKVSCKASGYTFTSYYMHWVRQAPGQGLEWMGWMNPNSGHTGYAQKFQGRVTMTRDTSTSTVYMELSSLRSEDTAVYYCAREDFYGDFDYWGQGTLVTVSS

SEQ ID NO: 255 - protein, 118 aa, synthetic construct
  REGION 1..118: note = Description of Artificial Sequence: Synthetic polypeptide
QVQLVQSGAEVKKPGASVKVSCKASGYTFTRHFIHWVRQAPGQGLEWMGWIDPNSGVTSSAQKFQGRVTMTRDTSTSTVYMELSSLRSEDTAVYYCARELSRWGFDYWGPGTMVTVSS

SEQ ID NO: 256 - protein, 118 aa, synthetic construct
  REGION 1..118: note = Description of Artificial Sequence: Synthetic polypeptide
QVQLVQSGAEVKKPGASVKVSCKASGYTFTRHLLHWVRQAPGQGLEWMGWISPQHGVRNYAHKFQGRVTMTRDTSTSTVYMELSSLRSEDTAVYYCARESVEGYFDLWGRGTLVTVSS

SEQ ID NO: 257 - protein, 123 aa, synthetic construct
  REGION 1..123: note = Description of Artificial Sequence: Synthetic polypeptide
QVQLVQSGAEVKKPGASVKVSCKASGYPFSSYYMHWVRQAPGQGLEWMGMINPSGGSTSYAQKFQGRVTMTRDTSTSTVYMELSSLRSEDTAVYYCARDIFPTMIAGGGFDLWGRGTLVTVSS

SEQ ID NO: 258 - protein, 119 aa, synthetic construct
  REGION 1..119: note = Description of Artificial Sequence: Synthetic polypeptide
QVQLVQSGAEVKKPGSSVKVSCKASGGTFSTFGISWVRQAPGQGLEWMGGIIPIFGTANYAQKFQGRVTITADESTSTAYMELSSLRSEDTAVYYCARGGYSYGSFDYWGQGTLVTVSS

SEQ ID NO: 259 - protein, 124 aa, synthetic construct
  REGION 1..124: note = Description of Artificial Sequence: Synthetic polypeptide
QVQLVQSGAEVKKPGASVKVSCKASGYTFTSYGINWVRQAPGQGLEWMGWMNPNSGNTGYAQKFQGRVTMTRDTSTSTVYMELSSLRSEDTAVYYCARGSFPLVFTIFGVGDVWGQGTMVTVSS

SEQ ID NO: 260 - protein, 119 aa, synthetic construct
  REGION 1..119: note = Description of Artificial Sequence: Synthetic polypeptide
QVQLVQSGAEVKKPGASVKVSCKASGYTFTSYYMHWVRQAPGQGLEWMGWISPRSGVTSYAQKFQGRVTMTRDTSTSTVYMELSSLRSEDTAVYYCARDLDYVRAFDIWGQGTTVTVSS

SEQ ID NO: 261 - protein, 118 aa, synthetic construct
  REGION 1..118: note = Description of Artificial Sequence: Synthetic polypeptide
QVQLVQSGAEVKKPGASVKVSCKASGYSFTSYYMHWVRQAPGQGLEWMGWMDPNSGNTGYAQKFQGRVTMTRDTSTSTVYMELSSLRSEDTAVYYCARESWGGYFDLWGRGTLVTVSS

SEQ ID NO: 262 - protein, 118 aa, synthetic construct
  REGION 1..118: note = Description of Artificial Sequence: Synthetic polypeptide
QVQLVQSGAEVKKPGASVKVSCKASGYTFINHYVHWVRQAPGQGLEWMGWMNPTGGITGYAQKFQGRVTMTRDTSTSTVYMELSSLRSEDTAVYYCARDRTTYAFDIWGQGTMVTVSS

SEQ ID NO: 263 - protein, 118 aa, synthetic construct; identical to SEQ ID NO: 243
  REGION 1..118: note = Description of Artificial Sequence: Synthetic polypeptide

SEQ ID NO: 264 - protein, 120 aa, synthetic construct
  REGION 1..120: note = Description of Artificial Sequence: Synthetic polypeptide
QVQLVQSGAEVKKPGSSVKVSCKASGYTFTRHLLHWVRQAPGQGLEWMGWVSPIHGLTGYAPRFQGRVTITADESTSTAYMELSSLRSEDTAVYYCARVHGSGSDGMDVWGQGTTVTVSS

SEQ ID NO: 265 - protein, 107 aa, synthetic construct
  REGION 1..107: note = Description of Artificial Sequence: Synthetic polypeptide
DIQMTQSPSSLSASVGDRVTITCRASQGIDSYLAWYQQKPGKAPKLLIYAASSLQSGVPSRFSGSGSGTDFTLTISSLQPEDFATYYCQQSYSTPITFGGGTKVEIK

SEQ ID NO: 266 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYAASSLQSGVPSRFSGSGSGTDFTLTISSLQPEDFATYYCQQSYTTPITFGGGTKVEIK

SEQ ID NO: 267 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQIFSTPLTFGGGTKVEIK

SEQ ID NO: 268 - protein, 107 aa, synthetic construct
  REGION 1..107: note = Description of Artificial Sequence: Synthetic polypeptide
DIQMTQSPSSLSASVGDRVTITCRASQGISSYLAWYQQKPGKAPKLLIYGASNLQSGVPSRFSGSGSGTDFTLTISSLQPEDFATYYCQQSYSTPLTFGGGTKVEIK

SEQ ID NO: 269 - protein, 107 aa, synthetic construct
  REGION 1..107: note = Description of Artificial Sequence: Synthetic polypeptide
DIQMTQSPSSLSASVGDRVTITCRASQTISNYLNWYQQKPGKAPKLLIYAASSLQSGVPSRFSGSGSGTDFTLTISSLQPEDFATYYCQQSYTTPITFGGGTKVEIK

SEQ ID NO: 270 - protein, 108 aa, synthetic construct
  REGION 1..108: note = Description of Artificial Sequence: Synthetic polypeptide
EIVMTQSPATLSVSPGERATLSCRASQSVDRNYVTWYQQKPGQAPRLLIYGASTRATGIPARFSGSGSGTEFTLTISSLQSEDFAVYYCQQSYTTPYTFGQGTKVEIK

SEQ ID NO: 271 - protein, 107 aa, synthetic construct
  REGION 1..107: note = Description of Artificial Sequence: Synthetic polypeptide
DIQMTQSPSSLSASVGDRVTITCRASQGISQYLAWYQQKPGKAPKLLIYGASNLHSGVPSRFSGSGSGTDFTLTISSLQPEDFATYYCQQTFTTPLTFGGGTKVEIK

SEQ ID NO: 272 - protein, 107 aa, synthetic construct
  REGION 1..107: note = Description of Artificial Sequence: Synthetic polypeptide
DIQMTQSPSSLSASVGDRVTITCQASQDIGNYLNWYQQKPGKAPKLLIYAASSLESGVPSRFSGSGSGTDFTLTISSLQPEDFATYYCQQSYSTPLTFGGGTKVEIK

SEQ ID NO: 273 - protein, 107 aa, synthetic construct
  REGION 1..107: note = Description of Artificial Sequence: Synthetic polypeptide
DIQMTQSPSSLSASVGDRVTITCRASQGIRNDLGWYQQKPGKAPKLLIYSASNLQSGVPSRFSGSGSGTDFTLTISSLQPEDFATYYCQQANSFPFTFGGGTKVEIK

SEQ ID NO: 274 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYAASTLESGVPSRFSGSGSGTDFTLTISSLQPEDFATYYCQQSYTTPYSFGGGTKVEIK

SEQ ID NO: 275 - protein, 107 aa, synthetic construct
  REGION 1..107: note = Description of Artificial Sequence: Synthetic polypeptide
DIQMTQSPSSLSASVGDRVTITCRASQGISQYLAWYQQKPGKAPKLLIYGASNLHSGVPSRFSGSGSGTDFTLTISSLQPEDFATYYCQQTFITPLTFGGGTKVEIK

SEQ ID NO: 276 - protein, 106 aa, synthetic construct
  REGION 1..106: note = Description of Artificial Sequence: Synthetic polypeptide
DIQMTQSPSSLSASVGDRVTITCRASQIIGNYLAWYQQKPGKAPKLLIYHASILETGVPSRFSGSGSGTDFTLTISSLQPEDFATYYCQQSYSTPTFGGGTKVEIK

SEQ ID NO: 277 - protein, 107 aa, synthetic construct
  REGION 1..107: note = Description of Artificial Sequence: Synthetic polypeptide
DIQMTQSPSSLSASVGDRVTITCRASQIISSYLNWYQQKPGKAPKLLIYAASSLQSGVPSRFSGSGSGTDFTLTISSLQPEDFATYYCQQGFSTPFTFGGGTKVEIK

SEQ ID NO: 278 - protein, 107 aa, synthetic construct
  REGION 1..107: note = Description of Artificial Sequence: Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQSFTNPVTFGGGTKVEIK

SEQ ID NO: 279 - protein, 107 aa, synthetic construct
  REGION 1..107: note = Description of Artificial Sequence: Synthetic polypeptide
DIQMTQSPSSLSASVGDRVTITCRASQGISNYLAWYQQKPGKAPKLLIYAASTLQSGVPSRFSGSGSGTDFTLTISSLQPEDFATYYCQQSYSAPYTFGGGTKVEIK

SEQ ID NO: 280 - protein, 107 aa, synthetic construct
  REGION 1..107: note = Description of Artificial Sequence: Synthetic polypeptide
DIQMTQSPSSLSASVGDRVTITCRASQGISNNLNWYQQKPGKAPKLLIYAATTLQSGVPSRFSGSGSGTDFTLTISSLQPEDFATYYCQQSYSTPYTFGGGTKVEIK

SEQ ID NO: 281 - protein, 107 aa, synthetic construct [sequence omitted]

SEQ ID NO: 282 - protein, 107 aa, synthetic construct
  REGION 1..107: note = Description of Artificial Sequence: Synthetic polypeptide
DIQMTQSPSSLSASVGDRVTITCRASQGISNGLSWYQQKPGKAPKLLIYAASTLQSGVPSRFSGSGSGTDFTLTISSLQPEDFATYYCQQSHSTPLTFGGGTKVEIK

SEQ ID NO: 283 - protein, 107 aa, synthetic construct
  REGION 1..107: note = Description of Artificial Sequence: Synthetic polypeptide
DIQMTQSPSSLSASVGDRVTITCRASQSITGWLAWYQQKPGKAPKLLIYAASSLQSGVPSRFSGSGSGTDFTLTISSLQPEDFATYYCQQSYSTPYTFGQGTKVEIK

SEQ ID NO: 284 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYDATHLETGVPSRFSGSGSGTDFTLTISSLQPEDFATYYCQQSYSTPITFGQGTRLEIK

SEQ ID NO: 285 - protein, 107 aa, synthetic construct
  REGION 1..107: note = Description of Artificial Sequence: Synthetic polypeptide
DIQMTQSPSSLSASVGDRVTITCRASQGIRNDLAWYQQKPGKAPKLLIYAASTLQSGVPSRFSGSGSGTDFTLTISSLQPEDFATYYCQQSYSTPLTFGGGTKVEIK

SEQ ID NO: 286 - protein, 107 aa, synthetic construct
  REGION 1..107: note = Description of Artificial Sequence: Synthetic polypeptide
DIQMTQSPSSLSASVGDRVTITCQASQDISSYLNWYQQKPGKAPKLLIYAASSLQTGVPSRFSGSGSGTDFTLTISSLQPEDFATYYCQQSYSTPYTFGQGTKLEIK

SEQ ID NO: 287 - protein, 107 aa, synthetic construct
  REGION 1..107: note = Description of Artificial Sequence: Synthetic polypeptide
DIQMTQSPSSLSASVGDRVTITCRASQSITTYLNWYQQKPGKAPKLLIYAASSLQGGVPSRFSGSGSGTDFTLTISSLQPEDFATYYCQQSYSTPYTFGQGTKVEIK

SEQ ID NO: 288 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKLEIK

SEQ ID NO: 289 - protein, 107 aa, synthetic construct
  REGION 1..107: note = Description of Artificial Sequence: Synthetic polypeptide
DIQMTQSPSSLSASVGDRVTITCRASQSVSTWLAWYQQKPGKAPKLLIYAASNLESGVPSRFSGSGSGTDFTLTISSLQPEDFATYYCQQSYSTPYTFGQGTKLEIK

SEQ ID NO: 290 - protein, 107 aa, synthetic construct
  REGION 1..107: note = Description of Artificial Sequence: Synthetic polypeptide
DIQMTQSPSSLSASVGDRVTITCRASQSISNWLAWYQQKPGKAPKLLIYDVSHLESGVPSRFSGSGSGTDFTLTISSLQPEDFATYYCQQSYSTPFTFGPGTKVDIK

SEQ ID NO: 291 - protein, 107 aa, synthetic construct
  REGION 1..107: note = Description of Artificial Sequence: Synthetic polypeptide
DIQMTQSPSSLSASVGDRVTITCQASQGISNYLAWYQQKPGKAPKLLIYDASSLQSGVPSRFSGSGSGTDFTLTISSLQPEDFATYYCQQSYSTPLTFGGGTKVEIK

SEQ ID NO: 292 - protein, 108 aa, synthetic construct
  REGION 1..108: note = Description of Artificial Sequence: Synthetic polypeptide
EIVMTQSPATLSVSPGERATLSCRASQSLSSSSLAWYQQKPGQAPRLLIYGASTRATGIPARFSGSGSGTEFTLTISSLQSEDFAVYYCQQYGSSPFTFGPGTKVDIK

SEQ ID NO: 293 - protein, 107 aa, synthetic construct
  REGION 1..107: note = Description of Artificial Sequence: Synthetic polypeptide
DIQMTQSPSSLSASVGDRVTITCRASEHIANWLAWYQQKPGKAPKLLIYGVSSLESGVPSRFSGSGSGTDFTLTISSLQPEDFATYYCQQSYSTPYTFGQGTKLEIK

SEQ ID NO: 294 - protein, 107 aa, synthetic construct
  REGION 1..107: note = Description of Artificial Sequence: Synthetic polypeptide
DIQMTQSPSSLSASVGDRVTITCRASQSVGSWVAWYQQKPGKAPKLLIYPASTLQSGVPSRFSGSGSGTDFTLTISSLQPEDFATYYCQQSYSTPLTFGGGTKVEIK

SEQ ID NO: 295 - protein, 107 aa, synthetic construct
  REGION 1..107: note = Description of Artificial Sequence: Synthetic polypeptide
DIQMTQSPSSLSASVGDRVTITCRASQSISPWLAWYQQKPGKAPKLLIYDASNLETGVPSRFSGSGSGTDFTLTISSLQPEDFATYYCQQTYSTPITFGQGTRLEIK

SEQ ID NO: 296 - protein, 107 aa, synthetic construct
  REGION 1..107: note = Description of Artificial Sequence: Synthetic polypeptide
DIQMTQSPSSLSASVGDRVTITCRASQGISRYLAWYQQKPGKAPKLLIYAASTLQSGVPSRFSGSGSGTDFTLTISSLQPEDFATYYCQQSYSTPLTFGQGTKVEIK

SEQ ID NO: 297 - protein, 108 aa, synthetic construct
  REGION 1..108: note = Description of Artificial Sequence: Synthetic polypeptide
EIVMTQSPATLSVSPGERATLSCRASQTVSSNYLAWYQQKPGQAPRLLIYGASTRASGIPARFSGSGSGTEFTLTISSLQSEDFAVYYCQQYYTTPLTFGGGTKVEIK

SEQ ID NO: 298 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSFSTPLTFGQGTRLEIK

SEQ ID NO: 299 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYDASNLETGVPSRFSGSGSGTDFTLTISSLQPEDFATYYCQQSYSTPPTFGQGTKVEIK

SEQ ID NO: 300 - protein, 107 aa, synthetic construct
  REGION 1..107: note = Description of Artificial Sequence: Synthetic polypeptide
DIQMTQSPSSLSASVGDRVTITCRASQGISNNLNWYQQKPGKAPKLLIYATSTLQSGVPSRFSGSGSGTDFTLTISSLQPEDFATYYCQQSYSTPYTFGQGTKVEIK

SEQ ID NO: 301 - protein, 107 aa, synthetic construct
  REGION 1..107: note = Description of Artificial Sequence: Synthetic polypeptide
DIQMTQSPSSLSASVGDRVTITCRSSQGIRNDLSWYQQKPGKAPKLLIYLASNSHSGVPSRFSGSGSGTDFTLTISSLQPEDFATYYCLQHNSYPLTFGGGTKVEIK

SEQ ID NO: 302 - protein, 107 aa, synthetic construct
  REGION 1..107: note = Description of Artificial Sequence: Synthetic polypeptide
DIQMTQSPSSLSASVGDRVTITCRASQSISRWLAWYQQKPGKAPKLLIYAASSLQSGVPSRFSGSGSGTDFTLTISSLQPEDFATYYCQQSYSTPYTFGQGTKLEIK

SEQ ID NO: 303 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYDSSSLQTGVPSRFSGSGSGTDFTLTISSLQPEDFATYYCQQSYSTPVTFGQGTKVEIK

SEQ ID NO: 304 - protein, 107 aa, synthetic construct
  REGION 1..107: note = Description of Artificial Sequence: Synthetic polypeptide
DIQITHSPSSLSASVGYRLTITCRDSHSITTWLAWYHQKPWNAPKLMIYAASNLESGVPSRFSGSGSGTYFTLTISSLQPEDFATYYCQHFYNTQYTFGQGTKVEIK

SEQ ID NO: 305 - protein, 108 aa, synthetic construct
  REGION 1..108: note = Description of Artificial Sequence: Synthetic polypeptide
DIQMTQSPSSLSASVGDRVTITCRASQVIRNDLAWYQQKPGKAPKLLIYAASTLQSGVPSRFSGSGSGTDFTLTISSLQPEDFATYYCQQSLQYPSHFFGQGTKVEIK

SEQ ID NO: 306 - protein, 107 aa, synthetic construct
  REGION 1..107: note = Description of Artificial Sequence: Synthetic polypeptide
DIQMTQSPSSLSASVGDRVTITCRASQSISRYLNWYQQKPGKAPKLLIYAASTLQSGVPSRFSGSGSGTDFTLTISSLQPEDFAPYYCQQSYSTPLTFGPGTKVDIK

SEQ ID NO: 307 - protein, 290 aa, synthetic construct
  REGION 1..290: note = Description of Artificial Sequence: Synthetic polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 308 - protein, 5 aa, synthetic construct
  REGION 1..5: note = Description of Artificial Sequence: Synthetic peptide
SDYMH

SEQ ID NO: 309 - protein, 5 aa, synthetic construct
  REGION 1..5: note = Description of Artificial Sequence: Synthetic peptide
GYYMH

SEQ ID NO: 310 - protein, 5 aa, synthetic construct
  REGION 1..5: note = Description of Artificial Sequence: Synthetic peptide
SYYMH

SEQ ID NO: 311 - protein, 5 aa, synthetic construct
  REGION 1..5: note = Description of Artificial Sequence: Synthetic peptide
GYYIH

SEQ ID NO: 312 - protein, 5 aa, synthetic construct
  REGION 1..5: note = Description of Artificial Sequence: Synthetic peptide
THYMH

SEQ ID NO: 313 - protein, 5 aa, synthetic construct
  REGION 1..5: note = Description of Artificial Sequence: Synthetic peptide
SHDIN

SEQ ID NO: 314 - protein, 5 aa, synthetic construct
  REGION 1..5: note = Description of Artificial Sequence: Synthetic peptide
DHYLH

SEQ ID NO: 315 - protein, 5 aa, synthetic construct
  REGION 1..5: note = Description of Artificial Sequence: Synthetic peptide
NYYMH

SEQ ID NO: 316 - protein, 5 aa, synthetic construct
  REGION 1..5: note = Description of Artificial Sequence: Synthetic peptide
AYYVH

SEQ ID NO: 317 - protein, 5 aa, synthetic construct
  REGION 1..5: note = Description of Artificial Sequence: Synthetic peptide
RHYVH

SEQ ID NO: 318 - protein, 4 aa, synthetic construct
  REGION 1..4: note = Description of Artificial Sequence: Synthetic peptide
NYIH

SEQ ID NO: 319 - protein, 5 aa, synthetic construct
  REGION 1..5: note = Description of Artificial Sequence: Synthetic peptide
NHYVH

SEQ ID NO: 320 - protein, 5 aa, synthetic construct
  REGION 1..5: note = Description of Artificial Sequence: Synthetic peptide
SHYMH

SEQ ID NO: 321 - protein, 5 aa, synthetic construct
  REGION 1..5: note = Description of Artificial Sequence: Synthetic peptide
RHLLH

SEQ ID NO: 322 - protein, 17 aa, synthetic construct
  REGION 1..17: note = Description of Artificial Sequence: Synthetic peptide
WMSPYNGITGYAQKFQG

SEQ ID NO: 323 - protein, 17 aa, synthetic construct
  REGION 1..17: note = Description of Artificial Sequence: Synthetic peptide
WMSPSSGITGYAQKFQG

SEQ ID NO: 324 - protein, 17 aa, synthetic construct
  REGION 1..17: note = Description of Artificial Sequence: Synthetic peptide
WMTTNSGITGYAQKFQG

SEQ ID NO: 325 - protein, 17 aa, synthetic construct
  REGION 1..17: note = Description of Artificial Sequence: Synthetic peptide
GIIPIFGTASYAQKFQG

SEQ ID NO: 326 - protein, 17 aa, synthetic construct
  REGION 1..17: note = Description of Artificial Sequence: Synthetic peptide
WMNPNSGHAGSAQKFQG

SEQ ID NO: 327 - protein, 17 aa, synthetic construct
  REGION 1..17: note = Description of Artificial Sequence: Synthetic peptide
WMNPNSGNTGYAQKFQG

SEQ ID NO: 328 - protein, 17 aa, synthetic construct
  REGION 1..17: note = Description of Artificial Sequence: Synthetic peptide
WMNPNSGNTGYSQKFQG

SEQ ID NO: 329 - protein, 17 aa, synthetic construct
  REGION 1..17: note = Description of Artificial Sequence: Synthetic peptide
WMNPNIGNTGYAQKFQG

SEQ ID NO: 330 - protein, 17 aa, synthetic construct
  REGION 1..17: note = Description of Artificial Sequence: Synthetic peptide
WMNPNGGTTGYAQNFQG

SEQ ID NO: 331 - protein, 17 aa, synthetic construct
  REGION 1..17: note = Description of Artificial Sequence: Synthetic peptide
WMNPNRGITDSAQKFQG

SEQ ID NO: 332 - protein, 17 aa, synthetic construct
  REGION 1..17: note = Description of Artificial Sequence: Synthetic peptide
WMNPNSGSAGYAQKFQG

SEQ ID NO: 333 - protein, 17 aa, synthetic construct
  REGION 1..17: note = Description of Artificial Sequence: Synthetic peptide
WIHPRSGATGYAPKFQG

SEQ ID NO: 334 - protein, 17 aa, synthetic construct
  REGION 1..17: note = Description of Artificial Sequence: Synthetic peptide
WISPRSGVTSYAQKFQG

SEQ ID NO: 335 - protein, 17 aa, synthetic construct
  REGION 1..17: note = Description of Artificial Sequence: Synthetic peptide
WMDPNSGNTGYAQKFQG

SEQ ID NO: 336 - protein, 17 aa, synthetic construct
  REGION 1..17: note = Description of Artificial Sequence: Synthetic peptide
WMNPTGGITGYAQKFQG

SEQ ID NO: 337 - protein, 17 aa, synthetic construct
  REGION 1..17: note = Description of Artificial Sequence: Synthetic peptide
WMNPNSGHTGNAQKFQG

SEQ ID NO: 338 - protein, 17 aa, synthetic construct
  REGION 1..17: note = Description of Artificial Sequence: Synthetic peptide
WVSPIHGLTGYAPRFQG

SEQ ID NO: 339 - protein, 9 aa, synthetic construct
  REGION 1..9: note = Description of Artificial Sequence: Synthetic peptide
DRFSGSYDY

SEQ ID NO: 340 - protein, 7 aa, synthetic construct
  REGION 1..7: note = Description of Artificial Sequence: Synthetic peptide
DRGWFDP

SEQ ID NO: 341 - protein, 9 aa, synthetic construct
  REGION 1..9: note = Description of Artificial Sequence: Synthetic peptide
EGYSSGLDY

SEQ ID NO: 342 - protein, 11 aa, synthetic construct
  REGION 1..11: note = Description of Artificial Sequence: Synthetic peptide
DGRFWSGYPDY

SEQ ID NO: 343 - protein, 10 aa, synthetic construct
  REGION 1..10: note = Description of Artificial Sequence: Synthetic peptide
ESIAVAGYDY

SEQ ID NO: 344 - protein, 10 aa, synthetic construct
  REGION 1..10: note = Description of Artificial Sequence: Synthetic peptide
DRWYMGSADY

SEQ ID NO: 345 - protein, 10 aa, synthetic construct
  REGION 1..10: note = Description of Artificial Sequence: Synthetic peptide
DDWGGDWFDP

SEQ ID NO: 346 - protein, 10 aa, synthetic construct
  REGION 1..10: note = Description of Artificial Sequence: Synthetic peptide
ERLSVAGFDY

SEQ ID NO: 347 - protein, 10 aa, synthetic construct
  REGION 1..10: note = Description of Artificial Sequence: Synthetic peptide
EPLQLGGFDY

SEQ ID NO: 348 - protein, 10 aa, synthetic construct
  REGION 1..10: note = Description of Artificial Sequence: Synthetic peptide
EGFGPNAFDI

SEQ ID NO: 349 - protein, 10 aa, synthetic construct
  REGION 1..10: note = Description of Artificial Sequence: Synthetic peptide
DSWYGDWFDP

SEQ ID NO: 350 - protein, 9 aa, synthetic construct
  REGION 1..9: note = Description of Artificial Sequence: Synthetic peptide
EVIEVGMDV

SEQ ID NO: 351 - protein, 9 aa, synthetic construct
  REGION 1..9: note = Description of Artificial Sequence: Synthetic peptide
EAWFGELST

SEQ ID NO: 352 - protein, 9 aa, synthetic construct
  REGION 1..9: note = Description of Artificial Sequence: Synthetic peptide
EAYVAAFDI

SEQ ID NO: 353 - protein, 9 aa, synthetic construct
  REGION 1..9: note = Description of Artificial Sequence: Synthetic peptide
ERGYNAFDY

SEQ ID NO: 354 - protein, 8 aa, synthetic construct
  REGION 1..8: note = Description of Artificial Sequence: Synthetic peptide
DSVFGLDY

SEQ ID NO: 355 - protein, 10 aa, synthetic construct
  REGION 1..10: note = Description of Artificial Sequence: Synthetic peptide
DLDYVRAFDI

SEQ ID NO: 356 - protein, 9 aa, synthetic construct
  REGION 1..9: note = Description of Artificial Sequence: Synthetic peptide
ESWGGYFDL

SEQ ID NO: 357 - protein, 9 aa, synthetic construct
  REGION 1..9: note = Description of Artificial Sequence: Synthetic peptide
DRTTYAFDI

SEQ ID NO: 358 - protein, 9 aa, synthetic construct
  REGION 1..9: note = Description of Artificial Sequence: Synthetic peptide
GNWVDAFDI

SEQ ID NO: 359 - protein, 11 aa, synthetic construct
  REGION 1..11: note = Description of Artificial Sequence: Synthetic peptide
VHGSGSDGMDV

SEQ ID NO: 360 - protein, 11 aa, synthetic construct
  REGION 1..11: note = Description of Artificial Sequence: Synthetic peptide
RASQSISSWLA

SEQ ID NO: 361 - protein, 11 aa, synthetic construct
  REGION 1..11: note = Description of Artificial Sequence: Synthetic peptide
RASQSVGTWLA

SEQ ID NO: 362 - protein, 11 aa, synthetic construct
  REGION 1..11: note = Description of Artificial Sequence: Synthetic peptide
RASQGISNYLA

SEQ ID NO: 363 - protein, 11 aa, synthetic construct
  REGION 1..11: note = Description of Artificial Sequence: Synthetic peptide
RASQSISTWLA

SEQ ID NO: 364 - protein, 11 aa, synthetic construct
  REGION 1..11: note = Description of Artificial Sequence: Synthetic peptide
QASQDISNHLN

SEQ ID NO: 365 - protein, 11 aa, synthetic construct
  REGION 1..11: note = Description of Artificial Sequence: Synthetic peptide
RASQGISSWLA

SEQ ID NO: 366 - protein, 11 aa, synthetic construct
  REGION 1..11: note = Description of Artificial Sequence: Synthetic peptide
RASESISSWLA

SEQ ID NO: 367 - protein, 11 aa, synthetic construct
  REGION 1..11: note = Description of Artificial Sequence: Synthetic peptide
RASQSVGSWLA

SEQ ID NO: 368 - protein, 11 aa, synthetic construct
  REGION 1..11: note = Description of Artificial Sequence: Synthetic peptide
RASQNISNFLN

SEQ ID NO: 369 - protein, 12 aa, synthetic construct
  REGION 1..12: note = Description of Artificial Sequence: Synthetic peptide
RASQSLSSSYLA

SEQ ID NO: 370 - protein, 11 aa, synthetic construct
  REGION 1..11: note = Description of Artificial Sequence: Synthetic peptide
RASQSISSYLN

SEQ ID NO: 371 - protein, 11 aa, synthetic construct
  REGION 1..11: note = Description of Artificial Sequence: Synthetic peptide
RASQSISRWLA

SEQ ID NO: 372 - protein, 11 aa, synthetic construct
  REGION 1..11: note = Description of Artificial Sequence: Synthetic peptide
RDSHSITTWLA

SEQ ID NO: 373 - protein, 11 aa, synthetic construct
  REGION 1..11: note = Description of Artificial Sequence: Synthetic peptide
RASQVIRNDLA

SEQ ID NO: 374 - protein, 11 aa, synthetic construct
  REGION 1..11: note = Description of Artificial Sequence: Synthetic peptide
RASQSISRYLN

SEQ ID NO: 375 - protein, 7 aa, synthetic construct
  REGION 1..7: note = Description of Artificial Sequence: Synthetic peptide
AASSLQS

SEQ ID NO: 376 - protein, 7 aa, synthetic construct
  REGION 1..7: note = Description of Artificial Sequence: Synthetic peptide
AASTLEN

SEQ ID NO: 377 - protein, 7 aa, synthetic construct
  REGION 1..7: note = Description of Artificial Sequence: Synthetic peptide
RASNLES

SEQ ID NO: 378 - protein, 7 aa, synthetic construct
  REGION 1..7: note = Description of Artificial Sequence: Synthetic peptide
AASTLQR

SEQ ID NO: 379 - protein, 7 aa, synthetic construct
  REGION 1..7: note = Description of Artificial Sequence: Synthetic peptide
AASTLQS

SEQ ID NO: 380 - protein, 7 aa, synthetic construct
  REGION 1..7: note = Description of Artificial Sequence: Synthetic peptide
GASNLQR

SEQ ID NO: 381 - protein, 7 aa, synthetic construct
  REGION 1..7: note = Description of Artificial Sequence: Synthetic peptide
AASNLQS

SEQ ID NO: 382 - protein, 7 aa, synthetic construct
  REGION 1..7: note = Description of Artificial Sequence: Synthetic peptide
GASSLQS

SEQ ID NO: 383 - protein, 7 aa, synthetic construct
  REGION 1..7: note = Description of Artificial Sequence: Synthetic peptide
AASHLQS

SEQ ID NO: 384 - skipped

SEQ ID NO: 385 - protein, 7 aa, synthetic construct
  REGION 1..7: note = Description of Artificial Sequence: Synthetic peptide
DSSSLQT

SEQ ID NO: 386 - protein, 7 aa, synthetic construct
  REGION 1..7: note = Description of Artificial Sequence: Synthetic peptide
AASNLES

SEQ ID NO: 387 - protein, 9 aa, synthetic construct
  REGION 1..9: note = Description of Artificial Sequence: Synthetic peptide
QQSYSTPYT

SEQ ID NO: 388 - protein, 9 aa, synthetic construct
  REGION 1..9: note = Description of Artificial Sequence: Synthetic peptide
QQSFSTPYT

SEQ ID NO: 389 - protein, 9 aa, synthetic construct
  REGION 1..9: note = Description of Artificial Sequence: Synthetic peptide
QQSYSTPLT

SEQ ID NO: 390 - protein, 9 aa, synthetic construct
  REGION 1..9: note = Description of Artificial Sequence: Synthetic peptide
QQSYSTPFT

SEQ ID NO: 391 - protein, 9 aa, synthetic construct
  REGION 1..9: note = Description of Artificial Sequence: Synthetic peptide
QQSYSTPIT

SEQ ID NO: 392 - protein, 9 aa, synthetic construct
  REGION 1..9: note = Description of Artificial Sequence: Synthetic peptide
QQYYSTPYT

SEQ ID NO: 393 - protein, 9 aa, synthetic construct
  REGION 1..9: note = Description of Artificial Sequence: Synthetic peptide
QQSYSLPYT

SEQ ID NO: 394 - protein, 9 aa, synthetic construct
  REGION 1..9: note = Description of Artificial Sequence: Synthetic peptide
HQYFTTPLT

SEQ ID NO: 395 - protein, 9 aa, synthetic construct
  REGION 1..9: note = Description of Artificial Sequence: Synthetic peptide
QQSYSMPYT

SEQ ID NO: 396 - protein, 9 aa, synthetic construct
  REGION 1..9: note = Description of Artificial Sequence: Synthetic peptide
QQSYSTPVT

SEQ ID NO: 397 - protein, 9 aa, synthetic construct
  REGION 1..9: note = Description of Artificial Sequence: Synthetic peptide
QHFYNTQYT

SEQ ID NO: 398 - protein, 10 aa, synthetic construct
  REGION 1..10: note = Description of Artificial Sequence: Synthetic peptide
QQSLQYPSHF

SEQ ID NO: 399 - protein, 30 aa, synthetic construct; identical to SEQ ID NO: 187
  REGION 1..30: note = Description of Artificial Sequence: Synthetic polypeptide

SEQ ID NO: 400 - protein, 30 aa, synthetic construct; identical to SEQ ID NO: 178
  REGION 1..30: note = Description of Artificial Sequence: Synthetic polypeptide

SEQ ID NO: 401 - protein, 30 aa, synthetic construct; identical to SEQ ID NO: 191
  REGION 1..30: note = Description of Artificial Sequence: Synthetic polypeptide

SEQ ID NO: 402 - protein, 30 aa, synthetic construct
  REGION 1..30: note = Description of Artificial Sequence: Synthetic polypeptide
QVQLVQSGAEVKEPGASVKVSCKASGYTFT

SEQ ID NO: 403 - protein, 30 aa, synthetic construct
  REGION 1..30: note = Description of Artificial Sequence: Synthetic polypeptide
QVQLVQSGAEVKKPGASVKVSCKASGYNFS

SEQ ID NO: 404 - skipped

SEQ ID NO: 405 - protein, 30 aa, synthetic construct; identical to SEQ ID NO: 189
  REGION 1..30: note = Description of Artificial Sequence: Synthetic polypeptide

SEQ ID NO: 406 - protein, 30 aa, synthetic construct; identical to SEQ ID NO: 190
  REGION 1..30: note = Description of Artificial Sequence: Synthetic polypeptide

SEQ ID NO: 407 - protein, 14 aa, synthetic construct
  REGION 1..14: note = Description of Artificial Sequence: Synthetic peptide
WVRQAPGQGLEWMG

SEQ ID NO: 408 - protein, 14 aa, synthetic construct
  REGION 1..14: note = Description of Artificial Sequence: Synthetic peptide
WMRQAPGQGLEWIG

SEQ ID NO: 409 - protein, 32 aa, synthetic construct; identical to SEQ ID NO: 196
  REGION 1..32: note = Description of Artificial Sequence: Synthetic polypeptide

SEQ ID NO: 410 - protein, 32 aa, synthetic construct; identical to SEQ ID NO: 199
  REGION 1..32: note = Description of Artificial Sequence: Synthetic polypeptide

SEQ ID NO: 411 - protein, 11 aa, synthetic construct
  REGION 1..11: note = Description of Artificial Sequence: Synthetic peptide
WGQGTLVTVSS

SEQ ID NO: 412 - protein, 11 aa, synthetic construct
  REGION 1..11: note = Description of Artificial Sequence: Synthetic peptide
WGQGTTVTVSS

SEQ ID NO: 413 - protein, 11 aa, synthetic construct
  REGION 1..11: note = Description of Artificial Sequence: Synthetic peptide
WGRGTLVTVSS

SEQ ID NO: 414 - protein, 11 aa, synthetic construct
  REGION 1..11: note = Description of Artificial Sequence: Synthetic peptide
WGQGTMVTVSS

SEQ ID NO: 415 - protein, 23 aa, synthetic construct
  REGION 1..23: note = Description of Artificial Sequence: Synthetic peptide
DIQMTQSPSSLSASVGDRVTITC

SEQ ID NO: 416 - protein, 23 aa, synthetic construct
  REGION 1..23: note = Description of Artificial Sequence: Synthetic peptide
EIVMTQSPATLSVSPGERATLSC

SEQ ID NO: 417 - protein, 23 aa, synthetic construct
  REGION 1..23: note = Description of Artificial Sequence: Synthetic peptide
DIQITHSPSSLSASVGYRLTITC

SEQ ID NO: 418 - protein, 15 aa, synthetic construct
  REGION 1..15: note = Description of Artificial Sequence: Synthetic peptide
WYQQKPGKAPKLLIY

SEQ ID NO: 419 - protein, 15 aa, synthetic construct
  REGION 1..15: note = Description of Artificial Sequence: Synthetic peptide
WYQQKPGQAPRLLIY

SEQ ID NO: 420 - protein, 15 aa, synthetic construct
  REGION 1..15: note = Description of Artificial Sequence: Synthetic peptide
WYHQKPWNAPKLMIY

SEQ ID NO: 421 - protein, 32 aa, synthetic construct; identical to SEQ ID NO: 213
  REGION 1..32: note = Description of Artificial Sequence: Synthetic polypeptide

SEQ ID NO: 422 - protein, 32 aa, synthetic construct; identical to SEQ ID NO: 214
  REGION 1..32: note = Description of Artificial Sequence: Synthetic polypeptide

SEQ ID NO: 423 - protein, 32 aa, synthetic construct; identical to SEQ ID NO: 215
  REGION 1..32: note = Description of Artificial Sequence: Synthetic polypeptide

SEQ ID NO: 424 - protein, 32 aa, synthetic construct; identical to SEQ ID NO: 216
  REGION 1..32: note = Description of Artificial Sequence: Synthetic polypeptide

SEQ ID NO: 425 - protein, 10 aa, synthetic construct
  REGION 1..10: note = Description of Artificial Sequence: Synthetic peptide
FGGGTKVEIK

SEQ ID NO: 426 - protein, 10 aa, synthetic construct
  REGION 1..10: note = Description of Artificial Sequence: Synthetic peptide
FGQGTKLEIK

SEQ ID NO: 427 - protein, 10 aa, synthetic construct
  REGION 1..10: note = Description of Artificial Sequence: Synthetic peptide
FGQGTKVEIK

SEQ ID NO: 428 - protein, 10 aa, synthetic construct
  REGION 1..10: note = Description of Artificial Sequence: Synthetic peptide
FGQGTRLEIK

SEQ ID NO: 429 - protein, 10 aa, synthetic construct
  REGION 1..10: note = Description of Artificial Sequence: Synthetic peptide
FGPGTKVDIK

SEQ ID NO: 430 - protein, 118 aa, synthetic construct
  REGION 1..118: note = Description of Artificial Sequence: Synthetic polypeptide
QVQLVQSGAEVKKPGASVKVSCKASGYTFSSDYMHWVRQAPGQGLEWMGWMSPYNGITGYAQKFQGRVTMTRDTSTSTVYMELSSLRSEDTAVYYCARDRFSGSYDYWGQGTLVTVSS

SEQ ID NO: 431 - protein, 116 aa, synthetic construct
  REGION 1..116: note = Description of Artificial Sequence: Synthetic polypeptide
QVQLVQSGAEVKKPGASVKVSCKASGYTFTGYYMHWVRQAPGQGLEWMGWMSPSSGITGYAQKFQGRVTMTRDTSTSTVYMELSSLRSEDTAVYYCARDRGWFDPWGQGTLVTVSS

SEQ ID NO: 432 - protein, 118 aa, synthetic construct
  REGION 1..118: note = Description of Artificial Sequence: Synthetic polypeptide
QVQLVQSGAEVKKPGASVKVSCKASGYTFSSYYMHWVRQAPGQGLEWMGWMTTNSGITGYAQKFQGRVTMTRDTSTSTVYMELSSLRSEDTAVYYCAREGYSSGLDYWGQGTLVTVSS

SEQ ID NO: 433 - protein, 120 aa, synthetic construct
  REGION 1..120: note = Description of Artificial Sequence: Synthetic polypeptide
QVQLVQSGAEVKKPGSSVKVSCKASGYTFTGYYIHWVRQAPGQGLEWMGGIIPIFGTASYAQKFQGRVTITADESTSTAYMELSSLRSEDTAVYYCARDGRFWSGYPDYWGQGTLVTVSS

SEQ ID NO: 434 - protein, 119 aa, synthetic construct
  REGION 1..119: note = Description of Artificial Sequence: Synthetic polypeptide
QVQLVQSGAEVKKPGASVKVSCKASGYTFSTHYMHWVRQAPGQGLEWMGWMNPNSGHAGSAQKFQGRVTMTRDTSTSTVYMELSSLRSEDTAVYYCARESIAVAGYDYWGQGTLVTVSS

SEQ ID NO: 435 - protein, 119 aa, synthetic construct
  REGION 1..119: note = Description of Artificial Sequence: Synthetic polypeptide
QVQLVQSGAEVKKPGASVKVSCKASGYTFTSHDINWVRQAPGQGLEWMGWMNPNSGNTGYAQKFQGRVTMTRDTSTSTVYMELSSLRSEDTAVYYCARDRWYMGSADYWGQGTLVTVSS

SEQ ID NO: 436 - protein, 119 aa, synthetic construct
  REGION 1..119: note = Description of Artificial Sequence: Synthetic polypeptide
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARDDWGGDWFDPWGQGTLVTVSS

SEQ ID NO: 437 - protein, 119 aa, synthetic construct
  REGION 1..119: note = Description of Artificial Sequence: Synthetic polypeptide
QVQLVQSGAEVKKPGASVKVSCKASGYTFSTHYMHWVRQAPGQGLEWMGWMNPNSGNTGYSQKFQGRVTMTRDTSTSTVYMELSSLRSEDTAVYYCARERLSVAGFDYWGQGTLVTVSS

SEQ ID NO: 438 - protein, 119 aa, synthetic construct
  REGION 1..119: note = Description of Artificial Sequence: Synthetic polypeptide
QVQLVQSGAEVKEPGASVKVSCKASGYTFTDHYLHWVRQAPGQGLEWMGWMNPNIGNTGYAQKFQGRVTMTRDTSTSTVYMELSSLRSEDTAVYYCAREPLQLGGFDYWGQGTLVTVSS

SEQ ID NO: 439 - protein, 119 aa, synthetic construct
  REGION 1..119: note = Description of Artificial Sequence: Synthetic polypeptide
QVQLVQSGAEVKKPGASVKVSCKASGYTFTGYYMHWVRQAPGQGLEWMGWMNPNGGTTGYAQNFQGRVTMTRDTSTSTVYMELSSLRSEDTAVYYCAREGFGPNAFDIWGQGTTVTVSS

SEQ ID NO: 440 - protein, 119 aa, synthetic construct
  REGION 1..119: note = Description of Artificial Sequence: Synthetic polypeptide
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCARDSWYGDWFDPWGQGTLVTVSS

SEQ ID NO: 441 - protein, 118 aa, synthetic construct
  REGION 1..118: note = Description of Artificial Sequence: Synthetic polypeptide
QVQLVQSGAEVKKPGASVKVSCKASGYTFTGYYMHWMRQAPGQGLEWIGWMSPYNGITGYAQKFQGRVTMTRDTSTSTVYMELSSLRSEDTAVYYCAREVIEVGMDVWGQGTTVTVSS

SEQ ID NO: 442 - protein, 118 aa, synthetic construct
  REGION 1..118: note = Description of Artificial Sequence: Synthetic polypeptide
QVQLVQSGAEVKKPGASVKVSCKASGYTFTNYYMHWVRQAPGQGLEWMGWMSPSSGITGYAQKFQGRVTMTRDTSTSTVYMELSSLRSEDTAVYYCAREAWFGELSTWGQGTLVTVSS

SEQ ID NO: 443 - protein, 118 aa, synthetic construct
  REGION 1..118: note = Description of Artificial Sequence: Synthetic polypeptide
QVQLVQSGAEVKKPGASVKVSCKASGYNFSAYYVHWVRQAPGQGLEWMGWMNPNRGITDSAQKFQGRVTMTRDTSTSTVYMELSSLRSEDTAVYYCAREAYVAAFDIWGQGTTVTVSS

SEQ ID NO: 444 - protein, 118 aa, synthetic construct
  REGION 1..118: note = Description of Artificial Sequence: Synthetic polypeptide
QVQLVQSGAEVKKPGASVKVSCKASGYTFTRHYVHWVRQAPGQGLEWMGWMNPNSGSAGYAQKFQGRVTMTRDTSTSTVYMELSSLRSEDTAVYYCARERGYNAFDYWGQGTLVTVSS

SEQ ID NO: 445 - protein, 116 aa, synthetic construct
  REGION 1..116: note = Description of Artificial Sequence: Synthetic polypeptide
QVQLVQSGAEVKKPGASVKVSCKASGYTLPNYIHWVRQAPGQGLEWMGWIHPRSGATGYAPKFQGRVTMTRDTSTSTVYMELSSLRSEDTAVYYCARDSVFGLDYWGQGTLVTVSS

SEQ ID NO: 446 - protein, 119 aa, synthetic construct; identical to SEQ ID NO: 260
  REGION 1..119: note = Description of Artificial Sequence: Synthetic polypeptide

SEQ ID NO: 447 - protein, 118 aa, synthetic construct; identical to SEQ ID NO: 261
  REGION 1..118: note = Description of Artificial Sequence: Synthetic polypeptide

SEQ ID NO: 448 - protein, 118 aa, synthetic construct; identical to SEQ ID NO: 262
  REGION 1..118: note = Description of Artificial Sequence: Synthetic polypeptide

SEQ ID NO: 449 - protein, 118 aa, synthetic construct; identical to SEQ ID NO: 243
  REGION 1..118: note = Description of Artificial Sequence: Synthetic polypeptide

SEQ ID NO: 450 - protein, 120 aa, synthetic construct; identical to SEQ ID NO: 264
  REGION 1..120: note = Description of Artificial Sequence: Synthetic polypeptide

SEQ ID NO: 451 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYAASSLQSGVPSRFSGSGSGTDFTLTISSLQPEDFATYYCQQSYSTPYTFGGGTKVEIK

SEQ ID NO: 452 - protein, 107 aa, synthetic construct
  REGION 1..107: note = Description of Artificial Sequence: Synthetic polypeptide
DIQMTQSPSSLSASVGDRVTITCRASQSVGTWLAWYQQKPGKAPKLLIYAASTLENGVPSRFSGSGSGTDFTLTISSLQPEDFATYYCQQSFSTPYTFGGGTKVEIK

SEQ ID NO: 453 - protein, 107 aa, synthetic construct; identical to SEQ ID NO: 451
  REGION 1..107: note = Description of Artificial Sequence: Synthetic polypeptide

SEQ ID NO: 454 - protein, 107 aa, synthetic construct
  REGION 1..107: note = Description of Artificial Sequence: Synthetic polypeptide
DIQMTQSPSSLSASVGDRVTITCRASQGISNYLAWYQQKPGKAPKLLIYRASNLESGVPSRFSGSGSGTDFTLTISSLQPEDFATYYCQQSYSTPLTFGGGTKVEIK

SEQ ID NO: 455 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYAASTLQRGVPSRFSGSGSGTDFTLTISSLQPEDFATYYCQQSYSTPYTFGQGTKLEIK

SEQ ID NO: 456 - protein, 107 aa, synthetic construct
  REGION 1..107: note = Description of Artificial Sequence: Synthetic polypeptide
DIQMTQSPSSLSASVGDRVTITCRASQSISTWLAWYQQKPGKAPKLLIYAASTLQSGVPSRFSGSGSGTDFTLTISSLQPEDFATYYCQQSYSTPFTFGQGTKVEIK

SEQ ID NO: 457 - protein, 107 aa, synthetic construct
  REGION 1..107: note = Description of Artificial Sequence: Synthetic polypeptide
DIQMTQSPSSLSASVGDRVTITCQASQDISNHLNWYQQKPGKAPKLLIYGASNLQRGVPSRFSGSGSGTDFTLTISSLQPEDFATYYCQQSYSTPITFGQGTRLEIK

SEQ ID NO: 458 - protein, 107 aa, synthetic construct
  REGION 1..107: note = Description of Artificial Sequence: Synthetic polypeptide
DIQMTQSPSSLSASVGDRVTITCRASQGISSWLAWYQQKPGKAPKLLIYAASSLQSGVPSRFSGSGSGTDFTLTISSLQPEDFATYYCQQSYSTPYTFGQGTKVEIK

SEQ ID NO: 459 - protein, 107 aa, synthetic construct
  REGION 1..107: note = Description of Artificial Sequence: Synthetic polypeptide
DIQMTQSPSSLSASVGDRVTITCRASESISSWLAWYQQKPGKAPKLLIYAASSLQSGVPSRFSGSGSGTDFTLTISSLQPEDFATYYCQQSYSTPLTFGQGTKLEIK

SEQ ID NO: 460 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYAASNLQSGVPSRFSGSGSGTDFTLTISSLQPEDFATYYCQQYYSTPYTFGQGTKVEIK

SEQ ID NO: 461 - protein, 107 aa, synthetic construct
  REGION 1..107: note = Description of Artificial Sequence: Synthetic polypeptide
DIQMTQSPSSLSASVGDRVTITCRASQSVGSWLAWYQQKPGKAPKLLIYGASSLQSGVPSRFSGSGSGTDFTLTISSLQPEDFATYYCQQSYSTPLTFGGGTKVEIK

SEQ ID NO: 462 - protein, 107 aa, synthetic construct
  REGION 1..107: note = Description of Artificial Sequence: Synthetic polypeptide
DIQMTQSPSSLSASVGDRVTITCRASQSISSWLAWYQQKPGKAPKLLIYAASHLQSGVPSRFSGSGSGTDFTLTISSLQPEDFATYYCQQSYSTPYTFGQGTRLEIK

SEQ ID NO: 463 - protein, 107 aa, synthetic construct
  REGION 1..107: note = Description of Artificial Sequence: Synthetic polypeptide
DIQMTQSPSSLSASVGDRVTITCRASQNISNFLNWYQQKPGKAPKLLIYAASSLQSGVPSRFSGSGSGTDFTLTISSLQPEDFATYYCQQSYSLPYTFGQGTKVEIK

SEQ ID NO: 464 - protein, 107 aa, synthetic construct
  REGION 1..107: note = Description of Artificial Sequence: Synthetic polypeptide
DIQMTQSPSSLSASVGDRVTITCRASQGISSWLAWYQQKPGKAPKLLIYAASSLQSGVPSRFSGSGSGTDFTLTISSLQPEDFATYYCQQSYSTPLTFGGGTKVEIK

SEQ ID NO: 465 - protein, 108 aa, synthetic construct
  REGION 1..108: note = Description of Artificial Sequence: Synthetic polypeptide
EIVMTQSPATLSVSPGERATLSCRASQSLSSSYLAWYQQKPGQAPRLLIYGASTRATGIPARFSGSGSGTEFTLTISSLQSEDFAVYYCHQYFTTPLTFGQGTRLEIK

SEQ ID NO: 466 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASTLQSGVPSRFSGSGSGTDFTLTISSLQPEDFATYYCQQSYSMPYTFGQGTKLEIK

SEQ ID NO: 467 - protein, 107 aa, synthetic construct; identical to SEQ ID NO: 302
  REGION 1..107: note = Description of Artificial Sequence: Synthetic polypeptide

SEQ ID NO: 468 - protein, 107 aa, synthetic construct; identical to SEQ ID NO: 303
  REGION 1..107: note = Description of Artificial Sequence: Synthetic polypeptide

SEQ ID NO: 469 - protein, 107 aa, synthetic construct; identical to SEQ ID NO: 304
  REGION 1..107: note = Description of Artificial Sequence: Synthetic polypeptide

SEQ ID NO: 470 - protein, 108 aa, synthetic construct; identical to SEQ ID NO: 305
  REGION 1..108: note = Description of Artificial Sequence: Synthetic polypeptide

SEQ ID NO: 471 - protein, 107 aa, synthetic construct; identical to SEQ ID NO: 306
  REGION 1..107: note = Description of Artificial Sequence: Synthetic polypeptide

SEQ ID NO: 472 - protein, 106 aa, synthetic construct
  REGION 1..106: note = Description of Artificial Sequence: Synthetic polypeptide
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 473 - protein, 23 aa, synthetic construct
  REGION 1..23: note = Description of Artificial Sequence: Synthetic peptide
DIQLTQSPSSLSASVGDRVTITC

SEQ ID NO: 474 - protein, 11 aa, synthetic construct
  REGION 1..11: note = Description of Artificial Sequence: Synthetic peptide
KASQDVGTSVA

SEQ ID NO: 475 - protein, 15 aa, synthetic construct
  REGION 1..15: note = Description of Artificial Sequence: Synthetic peptide
WYQQKPGKAPKLLIY

SEQ ID NO: 476 - protein, 7 aa, synthetic construct
  REGION 1..7: note = Description of Artificial Sequence: Synthetic peptide
WTSTRHT

SEQ ID NO: 477 - protein, 32 aa, synthetic construct
  REGION 1..32: note = Description of Artificial Sequence: Synthetic polypeptide
GVPSRFSGSGSGTDFTFTISSLQPEDIATYYC

SEQ ID NO: 478 - protein, 8 aa, synthetic construct
  REGION 1..8: note = Description of Artificial Sequence: Synthetic peptide
QQYSLYRS

SEQ ID NO: 479 - protein, 10 aa, synthetic construct
  REGION 1..10: note = Description of Artificial Sequence: Synthetic peptide
FGQGTKVEIK

SEQ ID NO: 480 - protein, 119 aa, synthetic construct
  REGION 1..119: note = Description of Artificial Sequence: Synthetic polypeptide
EVQLVESGGGVVQPGRSLRLSCSSSGFDFTTYWMSWVRQAPGKGLEWVAEIHPDSSTINYAPSLKDRFTISRDNSKNTLFLQMDSLRPEDTGVYFCASLYFGFPWFAYWGQGTPVTVSS

SEQ ID NO: 481 - protein, 30 aa, synthetic construct
  REGION 1..30: note = Description of Artificial Sequence: Synthetic polypeptide
EVQLVESGGGVVQPGRSLRLSCSSSGFDFT

SEQ ID NO: 482 - protein, 5 aa, synthetic construct
  REGION 1..5: note = Description of Artificial Sequence: Synthetic peptide
TYWMS

SEQ ID NO: 483 - protein, 14 aa, synthetic construct
  REGION 1..14: note = Description of Artificial Sequence: Synthetic peptide
WVRQAPGKGLEWVA

SEQ ID NO: 484 - protein, 17 aa, synthetic construct
  REGION 1..17: note = Description of Artificial Sequence: Synthetic peptide
EIHPDSSTINYAPSLKD

SEQ ID NO: 485 - protein, 32 aa, synthetic construct
  REGION 1..32: note = Description of Artificial Sequence: Synthetic polypeptide
RFTISRDNSKNTLFLQMDSLRPEDTGVYFCAS

SEQ ID NO: 486 - protein, 10 aa, synthetic construct
  REGION 1..10: note = Description of Artificial Sequence: Synthetic peptide
LYFGFPWFAY

SEQ ID NO: 487 - protein, 11 aa, synthetic construct
  REGION 1..11: note = Description of Artificial Sequence: Synthetic peptide
WGQGTPVTVSS

SEQ ID NO: 488 - protein, 108 aa, synthetic construct
  REGION 1..108: note = Description of Artificial Sequence: Synthetic polypeptide
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 489 - protein, 23 aa, synthetic construct
  REGION 1..23: note = Description of Artificial Sequence: Synthetic peptide
DIQMTQSPSSLSASVGDRVTITC

SEQ ID NO: 490 - protein, 11 aa, synthetic construct
  REGION 1..11: note = Description of Artificial Sequence: Synthetic peptide
KASAAVGTYVA

SEQ ID NO: 491 - protein, 15 aa, synthetic construct
  REGION 1..15: note = Description of Artificial Sequence: Synthetic peptide
WYQQKPGKAPKLLIY

SEQ ID NO: 492 - protein, 7 aa, synthetic construct
  REGION 1..7: note = Description of Artificial Sequence: Synthetic peptide
SASYRKR

SEQ ID NO: 493 - protein, 32 aa, synthetic construct; identical to SEQ ID NO: 213
  REGION 1..32: note = Description of Artificial Sequence: Synthetic polypeptide

SEQ ID NO: 494 - protein, 10 aa, synthetic construct
  REGION 1..10: note = Description of Artificial Sequence: Synthetic peptide
HQYYTYPLFT

SEQ ID NO: 495 - protein, 10 aa, synthetic construct
  REGION 1..10: note = Description of Artificial Sequence: Synthetic peptide
FGQGTKLEIK

SEQ ID NO: 496 - protein, 30 aa, synthetic construct; identical to SEQ ID NO: 178
  REGION 1..30: note = Description of Artificial Sequence: Synthetic polypeptide

SEQ ID NO: 497 - protein, 5 aa, synthetic construct
  REGION 1..5: note = Description of Artificial Sequence: Synthetic peptide
EFGMN

SEQ ID NO: 498 - protein, 14 aa, synthetic construct
  REGION 1..14: note = Description of Artificial Sequence: Synthetic peptide
WVRQAPGQGLEWMG

SEQ ID NO: 499 - protein, 17 aa, synthetic construct
  REGION 1..17: note = Description of Artificial Sequence: Synthetic peptide
WINTKTGEATYVEEFKG

SEQ ID NO: 500 - protein, 32 aa, synthetic construct
  REGION 1..32: note = Description of Artificial Sequence: Synthetic polypeptide
RVTFTTDTSTSTAYMELRSLRSDDTAVYYCAR

SEQ ID NO: 501 - protein, 12 aa, synthetic construct
  REGION 1..12: note = Description of Artificial Sequence: Synthetic peptide
WDFAYYVEAMDY

SEQ ID NO: 502 - protein, 11 aa, synthetic construct
  REGION 1..11: note = Description of Artificial Sequence: Synthetic peptide
WGQGTTVTVSS

SEQ ID NO: 503 - protein, 106 aa, synthetic construct
  REGION 1..106: note = Description of Artificial Sequence: Synthetic polypeptide
ENVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIK

SEQ ID NO: 504 - protein, 23 aa, synthetic construct
  REGION 1..23: note = Description of Artificial Sequence: Synthetic peptide
ENVLTQSPSSMSASVGDRVNIAC

SEQ ID NO: 505 - protein, 10 aa, synthetic construct
  REGION 1..10: note = Description of Artificial Sequence: Synthetic peptide
SASSSVSYMH

SEQ ID NO: 506 - protein, 15 aa, synthetic construct
  REGION 1..15: note = Description of Artificial Sequence: Synthetic peptide
WFQQKPGKSPKLWIY

SEQ ID NO: 507 - protein, 7 aa, synthetic construct
  REGION 1..7: note = Description of Artificial Sequence: Synthetic peptide
STSNLAS

SEQ ID NO: 508 - protein, 32 aa, synthetic construct
  REGION 1..32: note = Description of Artificial Sequence: Synthetic polypeptide
GVPSRFSGSGSGTDYSLTISSMQPEDAATYYC

SEQ ID NO: 509 - protein, 9 aa, synthetic construct
  REGION 1..9: note = Description of Artificial Sequence: Synthetic peptide
QQRSSYPLT

SEQ ID NO: 510 - protein, 10 aa, synthetic construct
  REGION 1..10: note = Description of Artificial Sequence: Synthetic peptide
FGGGTKLEIK

SEQ ID NO: 511 - protein, 120 aa, synthetic construct
  REGION 1..120: note = Description of Artificial Sequence: Synthetic polypeptide
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 512 - protein, 30 aa, synthetic construct
  REGION 1..30: note = Description of Artificial Sequence: Synthetic polypeptide
QVKLEQSGAEVVKPGASVKLSCKASGFNIK

SEQ ID NO: 513 - protein, 5 aa, synthetic construct
  REGION 1..5: note = Description of Artificial Sequence: Synthetic peptide
DSYMH

SEQ ID NO: 514 - protein, 14 aa, synthetic construct
  REGION 1..14: note = Description of Artificial Sequence: Synthetic peptide
WLRQGPGQRLEWIG

SEQ ID NO: 515 - protein, 17 aa, synthetic construct
  REGION 1..17: note = Description of Artificial Sequence: Synthetic peptide
WIDPENGDTEYAPKFQG

SEQ ID NO: 516 - protein, 32 aa, synthetic construct
  REGION 1..32: note = Description of Artificial Sequence: Synthetic polypeptide
KATFTTDTSANTAYLGLSSLRPEDTAVYYCNE

SEQ ID NO: 517 - protein, 11 aa, synthetic construct
  REGION 1..11: note = Description of Artificial Sequence: Synthetic peptide
GTPTGPYYFDY

SEQ ID NO: 518 - protein, 11 aa, synthetic construct
  REGION 1..11: note = Description of Artificial Sequence: Synthetic peptide
WGQGTLVTVSS

SEQ ID NO: 519 - protein, 106 aa, synthetic construct
  REGION 1..106: note = Description of Artificial Sequence: Synthetic polypeptide
ENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 520 - protein, 23 aa, synthetic construct
  REGION 1..23: note = Description of Artificial Sequence: Synthetic peptide
ENVLTQSPSSMSVSVGDRVTIAC

SEQ ID NO: 521 - protein, 10 aa, synthetic construct
  REGION 1..10: note = Description of Artificial Sequence: Synthetic peptide
SASSSVPYMH

SEQ ID NO: 522 - protein, 15 aa, synthetic construct
  REGION 1..15: note = Description of Artificial Sequence: Synthetic peptide
WLQQKPGKSPKLLIY

SEQ ID NO: 523 - protein, 7 aa, synthetic construct
  REGION 1..7: note = Description of Artificial Sequence: Synthetic peptide
LTSNLAS

SEQ ID NO: 524 - protein, 32 aa, synthetic construct
  REGION 1..32: note = Description of Artificial Sequence: Synthetic polypeptide
GVPSRFSGSGSGTDYSLTISSVQPEDAATYYC

SEQ ID NO: 525 - protein, 9 aa, synthetic construct
  REGION 1..9: note = Description of Artificial Sequence: Synthetic peptide
QQRSSYPLT

SEQ ID NO: 526 - protein, 10 aa, synthetic construct
  REGION 1..10: note = Description of Artificial Sequence: Synthetic peptide
FGGGTKLEIK

SEQ ID NO: 527 - protein, 120 aa, synthetic construct; identical to SEQ ID NO: 511
  REGION 1..120: note = Description of Artificial Sequence: Synthetic polypeptide

SEQ ID NO: 528 - protein, 30 aa, synthetic construct; identical to SEQ ID NO: 512
  REGION 1..30: note = Description of Artificial Sequence: Synthetic polypeptide

SEQ ID NO: 529 - protein, 5 aa, synthetic construct
  REGION 1..5: note = Description of Artificial Sequence: Synthetic peptide
DSYMH

SEQ ID NO: 530 - protein, 14 aa, synthetic construct
  REGION 1..14: note = Description of Artificial Sequence: Synthetic peptide
WLRQGPGQRLEWIG

SEQ ID NO: 531 - protein, 17 aa, synthetic construct
  REGION 1..17: note = Description of Artificial Sequence: Synthetic peptide
WIDPENGDTEYAPKFQG

SEQ ID NO: 532 - protein, 32 aa, synthetic construct; identical to SEQ ID NO: 516
  REGION 1..32: note = Description of Artificial Sequence: Synthetic polypeptide

SEQ ID NO: 533 - protein, 11 aa, synthetic construct
  REGION 1..11: note = Description of Artificial Sequence: Synthetic peptide
GTPTGPYYFDY

SEQ ID NO: 534 - protein, 11 aa, synthetic construct
  REGION 1..11: note = Description of Artificial Sequence: Synthetic peptide
WGQGTLVTVSS

SEQ ID NO: 535 - protein, 23 aa, synthetic construct
  REGION 1..23: note = Description of Artificial Sequence: Synthetic peptide
QTVLSQSPAILSASPGEKVTMTC

SEQ ID NO: 536 - protein, 10 aa, synthetic construct
  REGION 1..10: note = Description of Artificial Sequence: Synthetic peptide
RASSSVTYIH

SEQ ID NO: 537 - protein, 15 aa, synthetic construct
  REGION 1..15: note = Description of Artificial Sequence: Synthetic peptide
WYQQKPGSSPKSWIY

SEQ ID NO: 538 - protein, 7 aa, synthetic construct
  REGION 1..7: note = Description of Artificial Sequence: Synthetic peptide
ATSNLAS

SEQ ID NO: 539 - protein, 32 aa, synthetic construct
  REGION 1..32: note = Description of Artificial Sequence: Synthetic polypeptide
GVPARFSGSGSGTSYSLTISRVEAEDAATYYC

SEQ ID NO: 540 - protein, 9 aa, synthetic construct
  REGION 1..9: note = Description of Artificial Sequence: Synthetic peptide
QHWSSKPPT

SEQ ID NO: 541 - protein, 10 aa, synthetic construct
  REGION 1..10: note = Description of Artificial Sequence: Synthetic peptide
FGGGTKLEIK

SEQ ID NO: 542 - protein, 121 aa, synthetic construct
  REGION 1..121: note = Description of Artificial Sequence: Synthetic polypeptide
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 543 - protein, 30 aa, synthetic construct
  REGION 1..30: note = Description of Artificial Sequence: Synthetic polypeptide
EVKLVESGGGLVQPGGSLRLSCATSGFTFT

SEQ ID NO: 544 - protein, 5 aa, synthetic construct
  REGION 1..5: note = Description of Artificial Sequence: Synthetic peptide
DYYMN

SEQ ID NO: 545 - protein, 14 aa, synthetic construct
  REGION 1..14: note = Description of Artificial Sequence: Synthetic peptide
WVRQPPGKALEWLG

SEQ ID NO: 546 - protein, 19 aa, synthetic construct
  REGION 1..19: note = Description of Artificial Sequence: Synthetic peptide
FIGNKANGYTTEYSASVKG

SEQ ID NO: 547 - protein, 32 aa, synthetic construct
  REGION 1..32: note = Description of Artificial Sequence: Synthetic polypeptide
RFTISRDKSQSILYLQMNTLRAEDSATYYCTR

SEQ ID NO: 548 - protein, 10 aa, synthetic construct
  REGION 1..10: note = Description of Artificial Sequence: Synthetic peptide
DRGLRFYFDY

SEQ ID NO: 549 - protein, 11 aa, synthetic construct
  REGION 1..11: note = Description of Artificial Sequence: Synthetic peptide
WGQGTTLTVSS

SEQ ID NO: 550 - protein, 111 aa, synthetic construct
  REGION 1..111: note = Description of Artificial Sequence: Synthetic polypeptide
DIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIK

SEQ ID NO: 551 - protein, 23 aa, synthetic construct
  REGION 1..23: note = Description of Artificial Sequence: Synthetic peptide
DIQLTQSPSSLSASVGDRVTITC

SEQ ID NO: 552 - protein, 15 aa, synthetic construct
  REGION 1..15: note = Description of Artificial Sequence: Synthetic peptide
RAGESVDIFGVGFLH

SEQ ID NO: 553 - protein, 15 aa, synthetic construct
  REGION 1..15: note = Description of Artificial Sequence: Synthetic peptide
WYQQKPGKAPKLLIY

SEQ ID NO: 554 - protein, 7 aa, synthetic construct
  REGION 1..7: note = Description of Artificial Sequence: Synthetic peptide
RASNLES

SEQ ID NO: 555 - protein, 32 aa, synthetic construct
  REGION 1..32: note = Description of Artificial Sequence: Synthetic polypeptide
GVPSRFSGSGSRTDFTLTISSLQPEDFATYYC

SEQ ID NO: 556 - protein, 9 aa, synthetic construct
  REGION 1..9: note = Description of Artificial Sequence: Synthetic peptide
QQTNEDPYT

SEQ ID NO: 557 - protein, 10 aa, synthetic construct
  REGION 1..10: note = Description of Artificial Sequence: Synthetic peptide
FGQGTKVEIK

SEQ ID NO: 558 - protein, 121 aa, synthetic construct
  REGION 1..121: note = Description of Artificial Sequence: Synthetic polypeptide
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSS

SEQ ID NO: 559 - protein, 30 aa, synthetic construct
  REGION 1..30: note = Description of Artificial Sequence: Synthetic polypeptide
EVQLVESGGGLVQPGGSLRLSCAASGFNIK

SEQ ID NO: 560 - protein, 5 aa, synthetic construct
  REGION 1..5: note = Description of Artificial Sequence: Synthetic peptide
DTYMH

SEQ ID NO: 561 - protein, 14 aa, synthetic construct
  REGION 1..14: note = Description of Artificial Sequence: Synthetic peptide
WVRQAPGKGLEWVA

SEQ ID NO: 562 - protein, 17 aa, synthetic construct
  REGION 1..17: note = Description of Artificial Sequence: Synthetic peptide
RIDPANGNSKYADSVKG

SEQ ID NO: 563 - protein, 32 aa, synthetic construct
  REGION 1..32: note = Description of Artificial Sequence: Synthetic polypeptide
RFTISADTSKNTAYLQMNSLRAEDTAVYYCAP

SEQ ID NO: 564 - protein, 12 aa, synthetic construct
  REGION 1..12: note = Description of Artificial Sequence: Synthetic peptide
FGYYVSDYAMAY

SEQ ID NO: 565 - protein, 11 aa, synthetic construct
  REGION 1..11: note = Description of Artificial Sequence: Synthetic peptide
WGQGTLVTVSS

SEQ ID NO: 566 - protein, 107 aa, synthetic construct [sequence omitted]

SEQ ID NO: 567 - protein, 23 aa, synthetic construct
  REGION 1..23: note = Description of Artificial Sequence: Synthetic peptide
DIQMTQSPASLSASVGDRVTITC

SEQ ID NO: 568 - protein, 11 aa, synthetic construct
  REGION 1..11: note = Description of Artificial Sequence: Synthetic peptide
RASENIFSYLA

SEQ ID NO: 569 - protein, 15 aa, synthetic construct
  REGION 1..15: note = Description of Artificial Sequence: Synthetic peptide
WYQQKPGKSPKLLVY

SEQ ID NO: 570 - protein, 7 aa, synthetic construct
  REGION 1..7: note = Description of Artificial Sequence: Synthetic peptide
NTRTLAE

SEQ ID NO: 571 - protein, 32 aa, synthetic construct
  REGION 1..32: note = Description of Artificial Sequence: Synthetic polypeptide
GVPSRFSGSGSGTDFSLTISSLQPEDFATYYC

SEQ ID NO: 572 - protein, 9 aa, synthetic construct
  REGION 1..9: note = Description of Artificial Sequence: Synthetic peptide
QHHYGTPFT

SEQ ID NO: 573 - protein, 10 aa, synthetic construct
  REGION 1..10: note = Description of Artificial Sequence: Synthetic peptide
FGSGTKLEIK

SEQ ID NO: 574 - protein, 120 aa, synthetic construct [sequence omitted]

SEQ ID NO: 575 - protein, 30 aa, synthetic construct
  REGION 1..30: note = Description of Artificial Sequence: Synthetic polypeptide
EVQLQESGPGLVKPGGSLSLSCAASGFVFS

SEQ ID NO: 576 - protein, 5 aa, synthetic construct
  REGION 1..5: note = Description of Artificial Sequence: Synthetic peptide
SYDMS

SEQ ID NO: 577 - protein, 14 aa, synthetic construct
  REGION 1..14: note = Description of Artificial Sequence: Synthetic peptide
WVRQTPERGLEWVA

SEQ ID NO: 578 - protein, 17 aa, synthetic construct
  REGION 1..17: note = Description of Artificial Sequence: Synthetic peptide
YISSGGGITYAPSTVKG

SEQ ID NO: 579 - protein, 32 aa, synthetic construct
  REGION 1..32: note = Description of Artificial Sequence: Synthetic polypeptide
RFTVSRDNAKNTLYLQMNSLTSEDTAVYYCAA

SEQ ID NO: 580 - protein, 11 aa, synthetic construct
  REGION 1..11: note = Description of Artificial Sequence: Synthetic peptide
HYFGSSGPFAY

SEQ ID NO: 581 - protein, 11 aa, synthetic construct
  REGION 1..11: note = Description of Artificial Sequence: Synthetic peptide
WGQGTLVTVSS

SEQ ID NO: 582 - protein, 116 aa, synthetic construct
  REGION 1..116: note = Description of Artificial Sequence: Synthetic polypeptide
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 583 - protein, 22 aa, synthetic construct
  REGION 1..22: note = Description of Artificial Sequence: Synthetic peptide
QAVLTQPASLSASPGASASLTC

SEQ ID NO: 584 - protein, 14 aa, synthetic construct
  REGION 1..14: note = Description of Artificial Sequence: Synthetic peptide
TLRRGINVGAYSIY

SEQ ID NO: 585 - protein, 15 aa, synthetic construct
  REGION 1..15: note = Description of Artificial Sequence: Synthetic peptide
WYQQKPGSPPQYLLR

SEQ ID NO: 586 - protein, 11 aa, synthetic construct
  REGION 1..11: note = Description of Artificial Sequence: Synthetic peptide
YKSDSDKQQGS

SEQ ID NO: 587 - protein, 34 aa, synthetic construct
  REGION 1..34: note = Description of Artificial Sequence: Synthetic polypeptide
GVSSRFSASKDASANAGILLISGLQSEDEADYYC

SEQ ID NO: 588 - protein, 10 aa, synthetic construct
  REGION 1..10: note = Description of Artificial Sequence: Synthetic peptide
MIWHSGASAV

SEQ ID NO: 589 - protein, 10 aa, synthetic construct
  REGION 1..10: note = Description of Artificial Sequence: Synthetic peptide
FGGGTKLTVL

SEQ ID NO: 590 - protein, 121 aa, synthetic construct
  REGION 1..121: note = Description of Artificial Sequence: Synthetic polypeptide
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 591 - protein, 30 aa, synthetic construct
  REGION 1..30: note = Description of Artificial Sequence: Synthetic polypeptide
EVQLVESGGGLVQPGRSLRLSCAASGFTVS

SEQ ID NO: 592 - protein, 5 aa, synthetic construct
  REGION 1..5: note = Description of Artificial Sequence: Synthetic peptide
SYWMH

SEQ ID NO: 593 - protein, 14 aa, synthetic construct
  REGION 1..14: note = Description of Artificial Sequence: Synthetic peptide
WVRQAPGKGLEWVG

SEQ ID NO: 594 - protein, 19 aa, synthetic construct
  REGION 1..19: note = Description of Artificial Sequence: Synthetic peptide
FIRNKANGGTTEYAASVKG

SEQ ID NO: 595 - protein, 19 aa, synthetic construct
  REGION 1..19: note = Description of Artificial Sequence: Synthetic peptide
FIRNKANSGTTEYAASVKG

SEQ ID NO: 596 - protein, 32 aa, synthetic construct
  REGION 1..32: note = Description of Artificial Sequence: Synthetic polypeptide
RFTISRDDSKNTLYLQMNSLRAEDTAVYYCAR

SEQ ID NO: 597 - protein, 10 aa, synthetic construct
  REGION 1..10: note = Description of Artificial Sequence: Synthetic peptide
DRGLRFYFDY

SEQ ID NO: 598 - protein, 11 aa, synthetic construct
  REGION 1..11: note = Description of Artificial Sequence: Synthetic peptide
WGQGTTVTVSS

SEQ ID NO: 599 - protein, 121 aa, synthetic construct
  REGION 1..121: note = Description of Artificial Sequence: Synthetic polypeptide
EVQLVESGGGLVQPGRSLRLSCAASGFTVSSYWMHWVRQAPGKGLEWVGFILNKANGGTTEYAASVKGRFTISRDDSKNTLYLQMNSLRAEDTAVYYCARDRGLRFYFDYWGQGTTVTVSS

SEQ ID NO: 600 - protein, 30 aa, synthetic construct; identical to SEQ ID NO: 591
  REGION 1..30: note = Description of Artificial Sequence: Synthetic polypeptide

SEQ ID NO: 601 - protein, 5 aa, synthetic construct
  REGION 1..5: note = Description of Artificial Sequence: Synthetic peptide
SYWMH

SEQ ID NO: 602 - protein, 14 aa, synthetic construct
  REGION 1..14: note = Description of Artificial Sequence: Synthetic peptide
WVRQAPGKGLEWVG

SEQ ID NO: 603 - protein, 19 aa, synthetic construct
  REGION 1..19: note = Description of Artificial Sequence: Synthetic peptide
FILNKANGGTTEYAASVKG

SEQ ID NO: 604 - protein, 32 aa, synthetic construct; identical to SEQ ID NO: 596
  REGION 1..32: note = Description of Artificial Sequence: Synthetic polypeptide

SEQ ID NO: 605 - protein, 10 aa, synthetic construct
  REGION 1..10: note = Description of Artificial Sequence: Synthetic peptide
DRGLRFYFDY

SEQ ID NO: 606 - protein, 11 aa, synthetic construct
  REGION 1..11: note = Description of Artificial Sequence: Synthetic peptide
WGQGTTVTVSS

SEQ ID NO: 607 - protein, 30 aa, synthetic construct
  REGION 1..30: note = Description of Artificial Sequence: Synthetic polypeptide
QVQLVQSGAEVKKPGASVKVSCKASGYTLP

SEQ ID NO: 608 - protein, 9 aa, synthetic construct
  REGION 1..9: note = Description of Artificial Sequence: Synthetic peptide
GGGGSSSSG